SEQUENCE LISTING (6 sequences)

SEQ ID NO: 1 - protein, 213 aa, synthetic construct
EIVLTQSPATLSLSPGERATLSCRASSRLIYMHWYQQKPGQAPRPLIYATSNLASGIPARFSGSGSGTDFTLTISSLEPEDFAVYYCQQWNSNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 452 aa, synthetic construct
QVQLVQSGAEVKKPGSSVKVSCKASGYTFTNYVMHWVRQAPGQGLEWMGYINPYNDDVKYNEKFKGRVTITADESTSTAYMELSSLRSEDTAVYYCARWGYYGSPLYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 218 aa, synthetic construct
DIQMTQSPSSLSASVGDRVTITCRASQSVSTSSYNYMHWYQQKPGKPPKLLIKYASNLESGVPSRFSGSGSGTDFTLTISSLQPEDFATYYCQHSWEIPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 452 aa, synthetic construct
EVQLVQSGAEVKKPGASVKVSCKASGYSFTDYYMHWVRQAPGQGLEWIGRINPYAGAAFYSQNFKDRVTLTVDTSTSTAYLELSSLRSEDTAVYYCAIERGADLEGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 220 aa, synthetic construct
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 449 aa, synthetic construct
QVQLVQSGPEVKKPGASVKVSCKASGYTFTDYAVHWVRQAPGKRLEWIGVISTYNDYTYNNQDFKGRVTMTRDTSASTAYMELSRLRSEDTAVYYCARGNSYFYALDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0115304 A1
(43) Pub. Date: Apr. 30, 2026

(54) BIOACTIVE CONJUGATES, PREPARATION METHOD AND USE THEREOF

(71) Applicant: BeOne Medicines I GmbH, Basel (CH)

(72) Inventors: Charng-Sheng Tsai, Beijing (CN); Mei-Hsuan Tsai, Beijing (CN); Maomao He, Beijing (CN); Zewei Wang, Beijing (CN); Xiaofan Li, Beijing (CN)

(73) Assignee: BeOne Medicines I GmbH, Basel (CH)

(21) Appl. No.: 19/431,869

(22) Filed: Dec. 23, 2025

Related U.S. Application Data
(63) Continuation of application No. PCT/CN2024/102428, filed on Jun. 28, 2024.

(30) Foreign Application Priority Data
Jun. 29, 2023 (WO) PCT/CN2023/104052

(51) Int. Cl. A61K 47/68; C07K 16/28

(52) U.S. Cl. CPC A61K 47/68031; A61K 47/6849; A61K 47/6889; C07K 16/2827; C07K 16/2851

(57) ABSTRACT

Compounds of formula (I) are provided, wherein Conjugator is a group capable of bonding a BA and joining the BA to the rest of the compound, Cleavable includes at least one site that can be cleaved by a β-glucuronidase enzyme, Spacer provides distance between Conjugator and Payload, Payload is a cytotoxic agent such as MMAE, and BA is a binding agent.

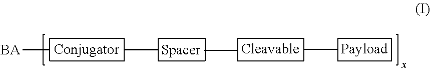

### CROSS REFERENCE TO RELATED APPLICATION

This application claims priority to International Application No. PCT/CN2023/104052, filed Jun. 29, 2023, the disclosure of which is hereby incorporated by reference in its entirety.

### FIELD

Provided herein are antibody drug conjugate platforms and antibody drug conjugates (ADCs) comprising the platforms and an antibody, or antigen-binding fragment thereof, as well as uses of the ADC platforms and ADCs.

### SEQUENCE LISTING

This application contains a Sequence Listing, which has been submitted electronically in XML format. The XML file is entitled “01368-0074-00PCT-ST26.xml,” was created on Jun. 20, 2024, and is 8,226 bytes in size. The Sequence Listing is incorporated herein by reference in its entirety.

### BACKGROUND

Antibody-drug conjugates (ADC) include an antibody against a tumor antigen linked to a biologically active small molecule such as a toxin or payload (i.e., a drug). ADCs selectively deliver the payload to cells expressing the tumor antigen. The payload monomethyl auristatin E (MMAE) is an antimitotic agent that inhibits cell division, ADCs including MMAE can be highly hydrophobic, leading to aggregation of ADCs with high drug-to-antibody ratios (DAR) and non-specific uptake of the ADCs. ADCs including MMAE can be unstable, leading to deconjugation, premature payload release, poor pharmacokinetic profiles, and off-target effects.

The linker component of ADCs, including the portion that directly conjugates with an antibody, is one important feature in developing optimized therapeutic agents that are highly active at well-tolerated doses. The electrophilic maleimide functional group has been used to join linkers with a free thiol of an antibody. In vivo, the conjugation product is subject to slow elimination, thus reversing the conjugation reaction and leaving the maleimide of an ADC free to transfer to any other available thiol, including those from serum albumin in plasma.

There is an ongoing need for the development of new linkers for use with MMAE in ADCs that would resist deconjugation and premature payload release, and allow for increased DAR, increased stability in circulation, improved pharmacokinetics, and improved efficacy. The present disclosure satisfies these needs.

### BRIEF SUMMARY

Provided herein are antibody drug conjugate platforms and antibody drug conjugates (ADCs). Also provided are uses of the ADC platforms to prepare ADCs.

In some embodiments, provided herein are ADC compounds of formula (I):

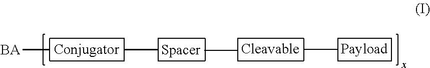

or a pharmaceutically acceptable salt, tautomer, solvate, or stereoisomer thereof, wherein:
BA is a binding agent selected from a humanized, monoclonal, chimeric, or human antibody or an antigen-binding fragment thereof;
Conjugator is selected from formula (II) and (III):

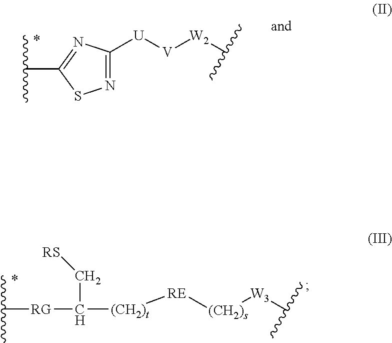

U is a bond, heteroarylene, or arylene;
V is a bond or —C═C—(CH₂)ₙ—;
n is an integer from 0 to 10 inclusive;
W₂ is —C(═O)—, —NH—, or —O—;
RG is

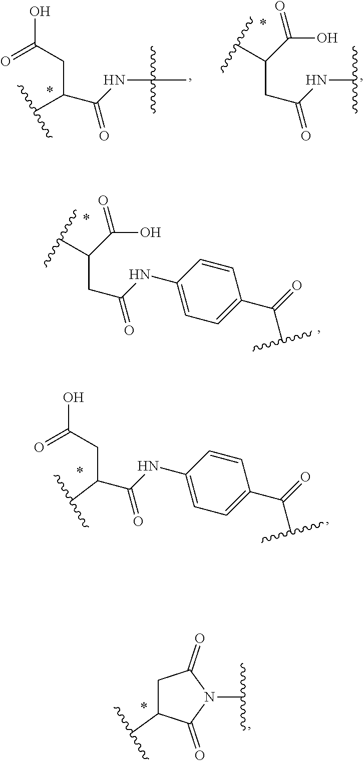 [SMILES: *NC(=O)C(*)CC(=O)O ; *NC(=O)CC(*)C(=O)O ; *C(=O)c1ccc(NC(=O)CC(*)C(=O)O)cc1 ; *C(=O)c1ccc(NC(=O)C(*)CC(=O)O)cc1 ; *C1CC(=O)N(*)C1=O]

-(succinimid-3-yl-N)—, or

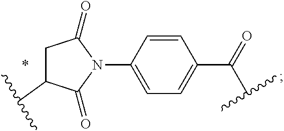 [SMILES: *C(=O)c1ccc(N2C(=O)CC(*)C2=O)cc1]

RS is —NR^{1a}R^{1b};
each of R^{1a} and R^{1b} is, independently, H or substituted or unsubstituted C₁₋₄ alkyl;
RE is a bond, —O—, —OC(═O)—, —OC(═O)NR⁶—, —NHC(═O)NR⁶—, —OS(═O)₂NR⁶—, —NHS(═O)₂NR⁶—, or —OC(═O)NHS(═O)₂NR⁶—.
R⁶ is H or substituted or unsubstituted C₁₋₄ alkyl;
W₃ is —C(═O)—, —NH—, or —O—;
each of s and t is, independently, 0, 1, or 2;
[symbol] indicates a point of covalent attachment within the compound;
* marks the bond where Conjugator connects to BA;
Spacer is a bond, **—NH—(CH₂CH₂O)ₘ—CH₂CH₂—C(═O)—, **—(CH₂)ₘ—C(═O)—, **—CH₂—C(═O)—NH—(CH₂)ₘ—C(═O)—, **—(CH₂CH₂O)ₘ—CH₂CH₂—C(═O)—, **—CH[—(CH₂)ₘ—COOH]—C(O)—, **—CH₂—C(═O)—NH—(CH₂)ₘ—C(═O)—NH—(CH₂)ₘC(═O)—, **—C(═O)—(CH₂)ₘ—C(O)—, **—C(═O)—(CH₂CH₂O)ₘ—CH₂CH₂—NH—, **—NH—(CH₂)ₘ—C(═O)—, or **—NH—(CH₂)ₘ—O—;
m is an integer from 1 to 12 inclusive;
* marks the bond where Spacer connects to Conjugator;
Cleavable has formula (IVa), (IVb), (TVc), (Va), (Vb), (VIa), (VIb), (VTTa), or (VIIb):

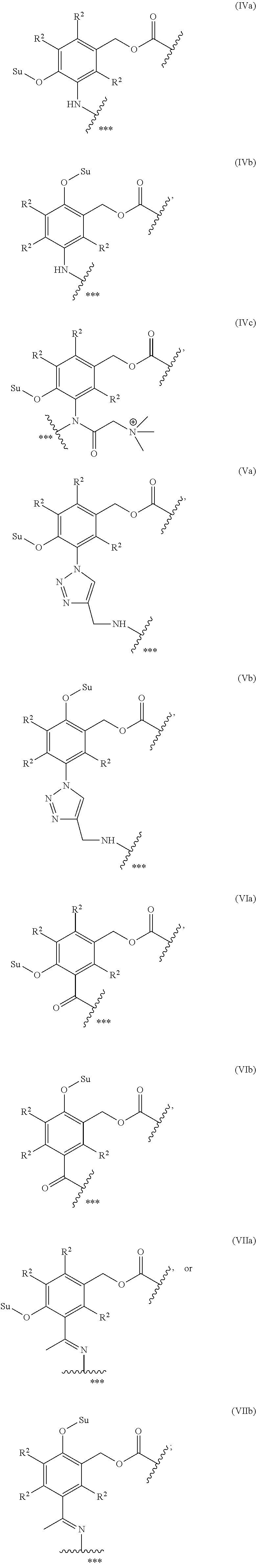

Su is a Sugar moiety;
each R² is, independently, hydrogen, halogen, substituted or unsubstituted C₁₋₄alkyl, —CN, or —NO₂;
*** marks the bond where Cleavable connects to Spacer;
Payload is a payload residue; and
x is from 1 to 15 inclusive.

In some embodiments, provided herein are ADC compounds of formula (XI):

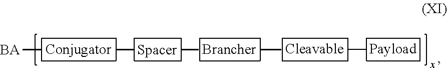

or a pharmaceutically acceptable salt, tautomer, solvate, or stereoisomer thereof, wherein:
BA is a binding agent selected from a humanized, monoclonal, chimeric, or human antibody or an antigen-binding fragment thereof;
Conjugator has formula (II) or (III):

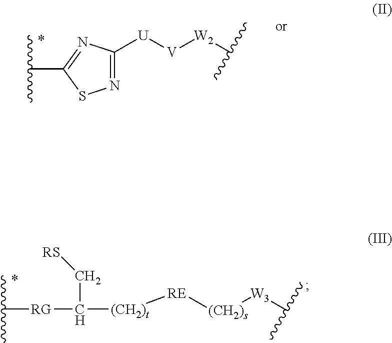

U is a bond, heteroarylene, or arylene;
V is a bond or —C═C—(CH₂)ₙ—;
n is an integer from 0 and 10 inclusive;
W₂ is —C(═O)—, —NH—, or —O—;
RG is

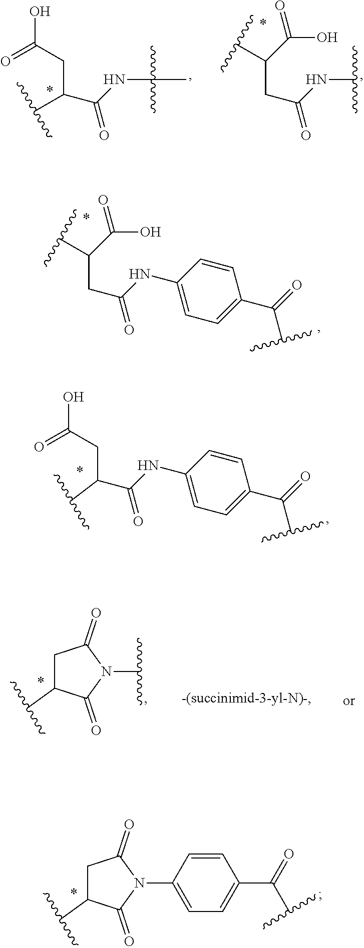 [SMILES: *NC(=O)C(*)CC(=O)O ; *NC(=O)CC(*)C(=O)O ; *C(=O)c1ccc(NC(=O)CC(*)C(=O)O)cc1 ; *C(=O)c1ccc(NC(=O)C(*)CC(=O)O)cc1 ; *C1CC(=O)N(*)C1=O ; *C(=O)c1ccc(N2C(=O)CC(*)C2=O)cc1]

RS is —NR^{1a}R^{1b};
each of R^{1a} and R^{1b} is, independently, H or substituted or unsubstituted C₁₋₄ alkyl;
RE is a bond, —O—, —OC(═O)—, —OC(═O)NR⁶—, —NHC(═O)NR⁶—, —OS(═O)₂NR⁶—, —NHS(═O)₂NR⁶—, or —OC(O)NHS(═O)₂NR⁶—;
R⁶ is H or substituted or unsubstituted C₁₋₄ alkyl;
W₃ is —C(═O)—, —NH—, or —O—;
each of s and t is, independently, 0, 1, or 2;
[symbol] indicates a point of covalent attachment within the compound;
* marks the bond where Conjugator connects to BA;
Spacer is a bond, **—NH—(CH₂CH₂O)ₘ—CH₂CH₂—C(═O)—, **—C(═O)—(CH₂CH₂O)ₘ—CH₂CH₂—NH—, **—(CH₂)ₘC(═O), **—CH₂—C(═O)—NH—(CH₂)ₘ—C(═O), **(CH₂CH₂O)ₘ—CH₂CH₂—C(═O)—, **—C(═O)—(CH₂CH₂O)ₘ—CH₂CH₂—NH—, **—CH[—(CH₂)ₘ—COOH]—C(═O)—, **—CH₂—C(═O)—NH—(CH₂)ₘ—C(═O)—NH—(CH₂)ₘ—C(═O)—, **—C(═O)—(CH₂)ₘ—C(═O)—, **—NH—CH₂)ₘ—C(═O)—, or **—NH—(CH₂)ₘ—O—;
m is an integer from 1 to 12 inclusive;
** marks the bond where Spacer connects to Conjugator;
Cleavable has formula (IVa′), (IVb′), (IVc′), (Va′), (Vb′), (Via′), (VIb′), (VIIa′), or (VIIb′):

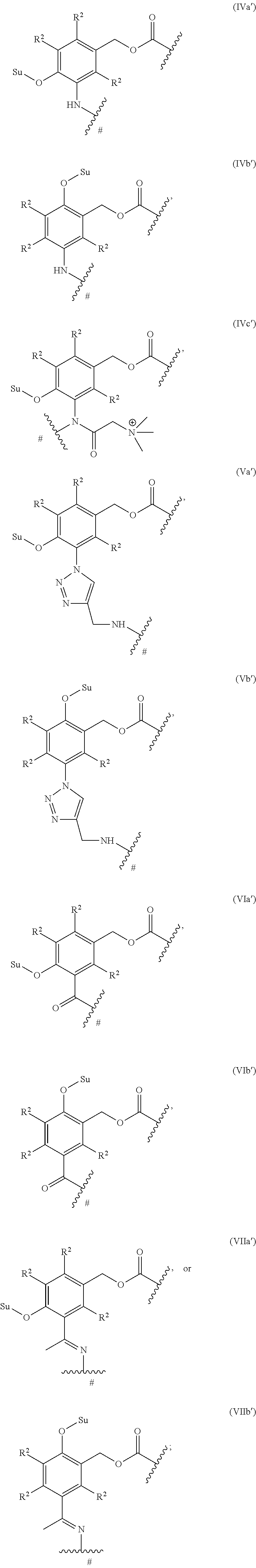

Su is a Sugar moiety;
each R² is, independently, hydrogen, halogen, substituted or unsubstituted C₁₋₄ alkyl, —CN, or —NO₂;
#marks the bond where Cleavable connects to Brancher;
Brancher is selected from formula (XIIa), (XIIb), (XIIc), and (XIId):

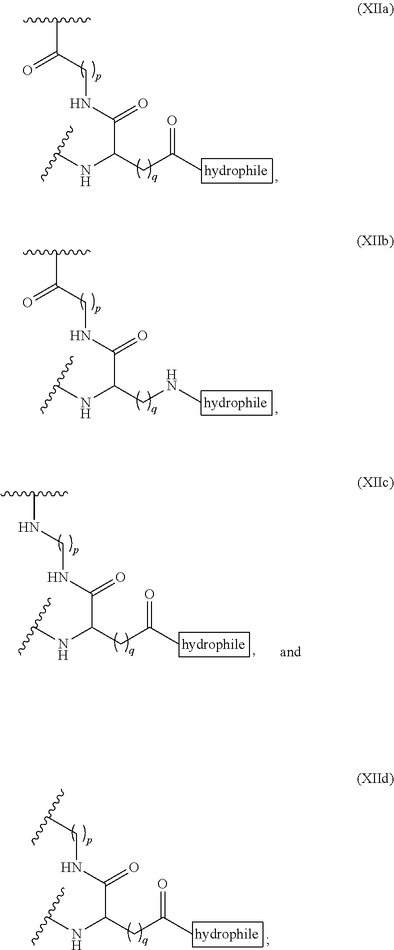

each of p and q is, independently, 1, 2, 3, or 4;
hydrophile is —NH—(CH₂CH₂O)ₐ—(CH₂)_{b}CH₃, —N(CH₃)—((CH₂)_{c}C(═O)N(CH₃))(CH₂)C(═O)NH₂, —C(═O)—(CH₂CH₂O)ₐ(CH₂)_{b}CH₃, —C(═O)—(CH₂CH₂O)ₐ—(CH₂)_{b}NHC(═O)(CH₂)₃N(CH₃)₂(CH₂)₃S(═O)₂OH, —NH—(CH₂CH₂O)ₐ—(CH₂)_{b}NHC(═O)(CH₂)₃N(CH₃)₂(CH₂)₃S(═O)₂OH,

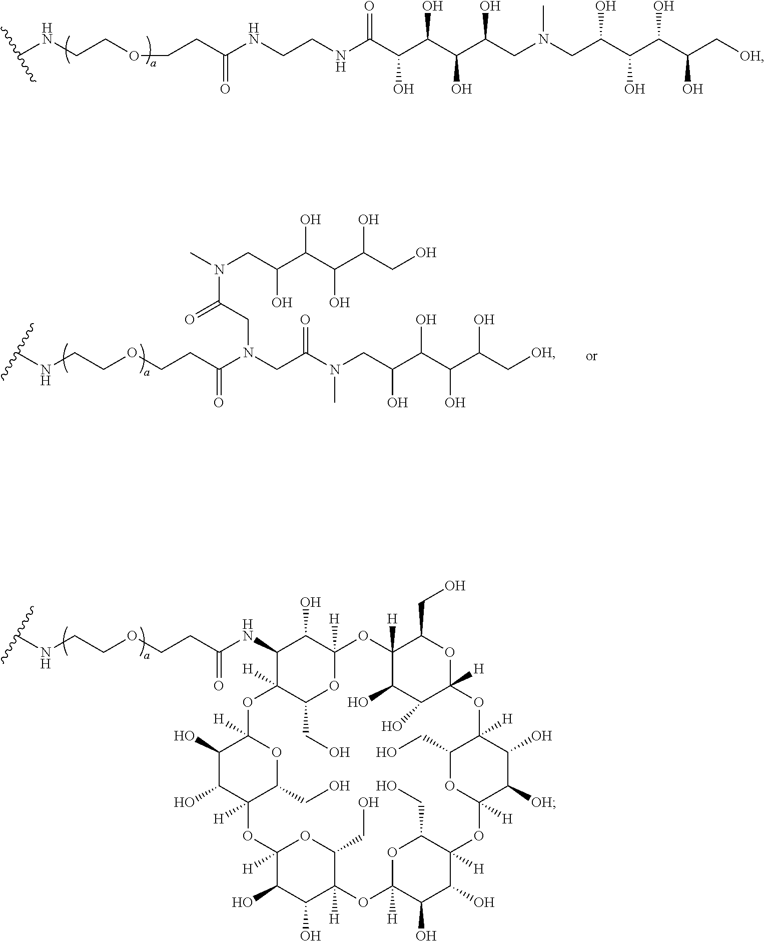

a is an integer from 1 to 18 inclusive;
b is 0, 1, or 2;
c is 1, 2, 3, or 4;
Payload is a payload residue; and
x is from 1 to 15 inclusive.

In some embodiments, the platform is a conjugator-linker-payload compound of formula (I-I):

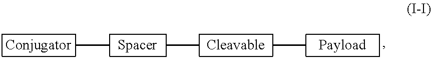

or a pharmaceutically acceptable salt, tautomer, solvate, or stereoisomer thereof wherein:
Conjugator has formula (I-II) or (I-III):

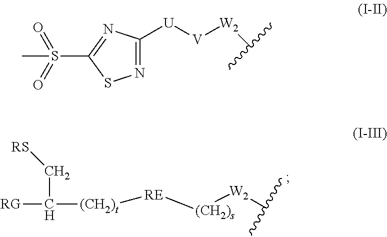

U is a bond, heteroarylene, or arylene;
V is a bond or —C≡C—(CH₂)ₙ—;
n is an integer from 0 to 10 inclusive;
W₂ is —C(═O)—, —NH—, or —O—;
RG is or

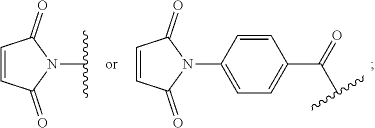 [SMILES: *N1C(=O)C=CC1=O ; *C(=O)c1ccc(N2C(=O)C=CC2=O)cc1]

RS is —NR^{1a}R^{1b};
each of R^{1a} and R^{1b} is, independently, 1H or substituted or unsubstituted C₁₋₄ alkyl;
RE is a bond, —O—, —OC(═O)—, —OC(═O)NR⁶—, —NHC(═O)NR⁶—, —OS(═O)₂NR⁶—, —NHS(═O)₂NR⁶—, or —OC(═O)NHS(═O)₂NR⁶—;
R⁶ is H or substituted or unsubstituted C₁₋₄ alkyl;
W₃ is —C(═O)—, —NH—, or —O—;
each of s and t is, independently, 0, 1, or 2;
[symbol] indicates a point of covalent attachment within the compound;
Spacer is a bond, **—NH—(CH₂CH₂O)ₘ—CH₂CH₂—C(═O)—, **—(CH₂)ₘ—C(═O)—**—CH₂—C(═O)—NH—(CH₂)ₘ—C(═O)—, **—(CH₂CH₂O)ₘ—CH₂CH₂—C(═O)—, **—CH[—(CH₂)ₘ—COOH]—C(═O)—, **—CH₂—C(═O)—NH—(CH₂)ₘ—C(═O)—NH—(CH₂)ₘ—C(═O)—, **—C(═O)—(CH₂)ₘ—C(═O)—, **—NH—(CH₂)ₘ—C(═O)— or **—NH—(CH₂)ₘ—O—;
m is an integer from 1 to 12 inclusive;
** marks the bond where Spacer connects to Conjugator;
Cleavable has formula (IVa), (IVb) (IVc), (Va), (Vb), (VIa), (VIb), (VIIa) or (VIIb):

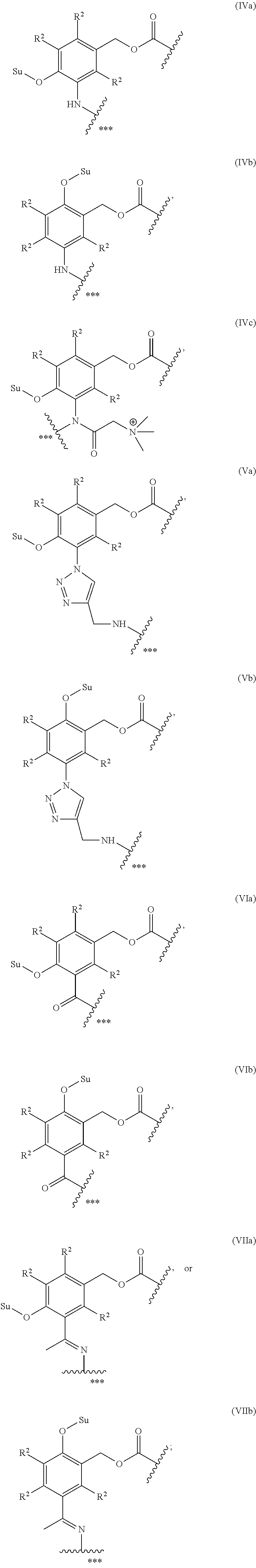

Sn is a Sugar moiety;
each R² is, independently, hydrogen, halogen, substituted or unsubstituted C₁₋₄ alkyl, —CN, or —NO₂;
*** marks the bond where Cleavable connects to Spacer; and
Payload is a payload residue.

In some embodiments, the platform is a conjugator-linker-payload compound of formula (XV):

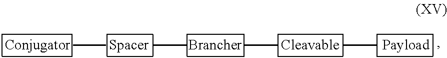

or a pharmaceutically acceptable salt, tautomer, solvate, or stereoisomer thereof, wherein:
Conjugator has formula (I-II) or (I-III):

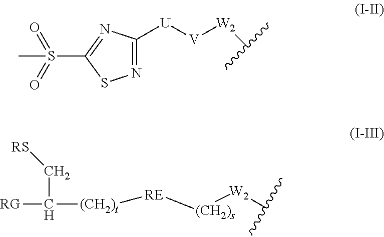

U is a bond, heteroarylene, or arylene;
V is a bond or —C≡C—(CH₂)ₙ—;
n is an integer from 0 to 10 inclusive;
W₂ is —C(═O)—, —NH—, or —O—;
RG is

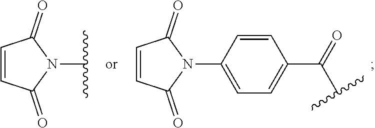 [SMILES: *N1C(=O)C=CC1=O ; *C(=O)c1ccc(N2C(=O)C=CC2=O)cc1]

RS is —NR^{1a}R^{1b};
each of R^{1a} and R^{1b} is, independently, H or substituted or unsubstituted C₁₋₄ alkyl;
RE is a bond, —O—, —OC(═O)—, —OC(═O)NR⁶—, —NHC(═O)NR⁶—, —OS(═O)₂NR⁶—, —NHS(═O)₂NR⁶—, or —OC(═O)NHS(═O)₂NR⁶—;
R is H or substituted or unsubstituted C₁₋₄ alkyl;
W₃ is —C(═O)—, —NH—, or —O—;
each of s and t is, independently, 0, 1, or 2;
[symbol] indicates a point of covalent attachment within the compound;
Spacer is a bond, **—NH—(CH₂CH₂O)ₘ—CH₂CH₂—C(═O)—, **—(CH₂)ₘ—C(═O)—, **—CH₂—C(═O)—NH—(CH₂)ₘ—C(═O)—, **—(CH₂CH₂O)ₘ—CH₂CH₂—C(═O)—, **—CH[—(CH₂)ₘ—COOH]—C(═O)—, **—CH₂—C(═O)—NH—(CH₂)ₘ—C(═O)—NH—(CH₂)ₘ—C(═O)—, **—C(═O)—(CH₂)ₘ—C(═O)—, **—NH—(CH₂)ₘ—C(═O)—, or **NH—(CH₂)ₘ—O—;
m is an integer from 1 to 12 inclusive;
** marks the bond where Spacer connects to Conjugator;
Cleavable has formula (IVa′), (IVb′), (IVc′), (Va′), (Vb′), (VIa′), (VIb′), (VIIa′), or (VIIb′):

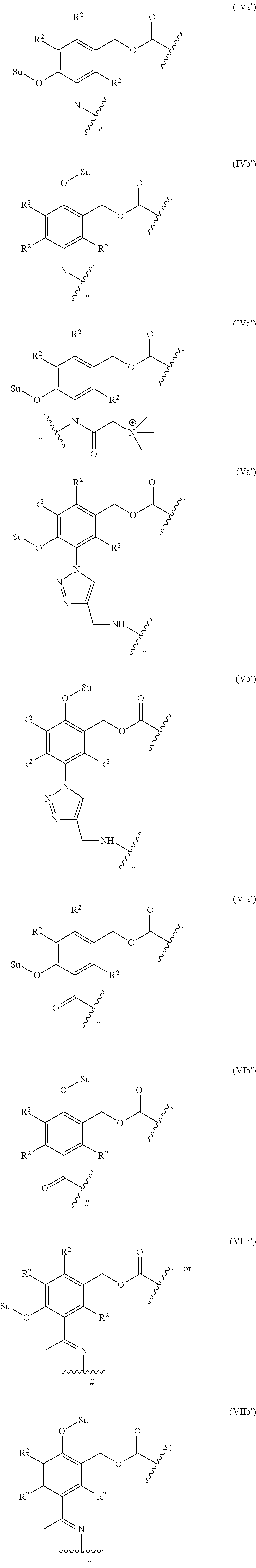

Su is a Sugar moiety;
each R² is, independently, hydrogen, halogen, substituted or unsubstituted C₁₋₄ alkyl, —CN, or —NO₂;
#marks the bond where Cleavable connects to Brancher;
Brancher is selected from formula (XIIa), (XIIb), (XIIc), and (XIId):

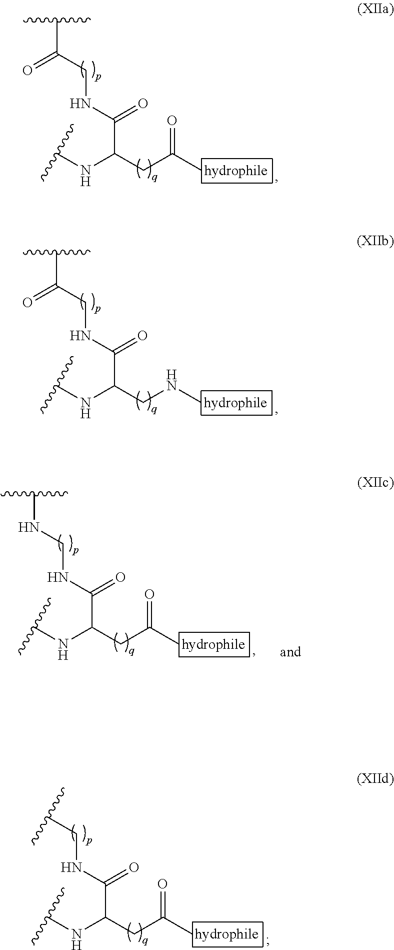

each of p and q is, independently, 1, 2, 3, or 4;
hydrophile is —NH—(CH₂CH₂O)ₐ—(CH₂)_{b}CH₃, —N(CH₃)—((CH₂)_{c}C(═O)N(CH₃))ₐ—(CH₂)_{c} C(═O)NH₂, —C(═O)—(CH₂CH₂O)ₐ—(CH₂)_{b}CH₃, —C(═O)—(CH₂CH₂O)ₐ—(CH₂)_{b}NHC(═O)(CH₂)₃N(CH₃)₂(CH₂)₃S(═O)₂OH, —NH—(CH₂CH₂O)ₐ—(CH₂)_{b}NHC(═O)(CH₂)₃N(CH₃)₂(CH₂)₃S(═O)₂OH,

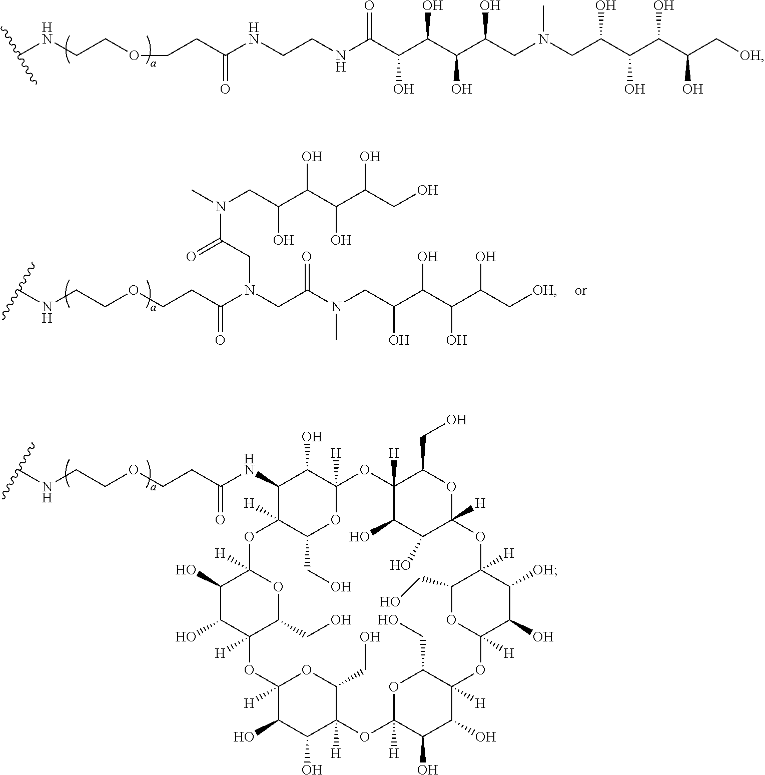

a is an integer from 1 to 18 inclusive;
b is 0, 1, or 2;
c is 1, 2, 3, or 4; and
Payload is a payload residue.

Additional objects and advantages will be set forth in part in the description that follows, and in part will be understood from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate embodiments and together with the description serve to explain the principles described herein.

### BRIEF DESCRIPTION OF FIGURES

FIGS. 1A-1I depict ADC cellular killing data on U937 cells.

FIGS. 2A-2I depict ADC cellular killing data on HL60 cells.

FIGS. 3A-3I depict ADC cellular killing data on TF1 cells.

FIGS. 4A-4B depict ADC cellular killing data on NCI-H358 cells.

FIGS. 5A-5B depict ADC cellular killing data on NCI-H1048 cells.

FIGS. 6A-6B depict ADC cellular killing data on MDA-MB-453 cells.

FIG. 7 shows ADC, payload, and total antibody (“TAB”) PK profiles in mice.

FIG. 8 shows anti-tumor efficacy of ADCs in an NCI-H1650 xenograft model.

### DETAILED DESCRIPTION

Provided herein are monomethyl auristatin E (MMAE)-based antibody drug conjugates (ADCs) and covalent linkers and conjugator-linker-payloads (platforms) for making such ADCs. The ADCs may be used to treat a disease or disorder, such as cancer, such as by providing a composition comprising an ADC. The presently disclosed MMAE ADCs can better resist deconjugation and premature payload release, and allow for increased DAR, increased stability in circulation, improved pharmacokinetics, and improved efficacy compared to known MMAE ADCs. The conjugates include a β-glucuronide-based linker comprising a site that can be cleaved by an enzyme having β-glucuronidase activity.

### Definitions

In the present disclosure, the following terms have the following meanings unless indicated otherwise. Unless defined otherwise, all technical and scientific terms used herein have the same meaning as is commonly understood by one of ordinary skill in the art to which this disclosure pertains. In the event that there is a plurality of definitions for a term provided herein, these Definitions prevail unless stated otherwise.

When a trade name is used herein, reference to the trade name also refers to the product formulation, the generic drug, and the active pharmaceutical ingredient(s) of the trade name product, unless otherwise indicated by context.

The term “antibody” herein is used in the broadest sense and specifically covers intact monoclonal antibodies, polyclonal antibodies, monospecific antibodies, multispecific antibodies (e.g., bispecific antibodies), and antibody fragments that exhibit the desired biological activity. An intact antibody has primarily two regions: a variable region and a constant region. The variable region binds to and interacts with a target antigen. The variable region includes a complementary determining region (CDR) that recognizes and binds to a specific binding site on a particular antigen. The constant region may be recognized by and interact with the immune system (see, e.g., Janeway et al., 2001, Immuno. Biology, 5^{th} Ed., Garland Publishing, New York). An antibody can be of any type (e.g., IgG, IgE, IgM, IgD, and IgA), class (e.g., IgG1, IgG2, IgG3, IgG4, IgA1, and IgA2) or subclass. The antibody can be derived from any suitable species. In some embodiments, the antibody is of human or murine origin. An antibody can be, for example, human, humanized, or chimeric.

The term “humanized” or “humanized antibody” means forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum,” “hu,” “Hu,” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions can be included to increase affinity, increase stability of the humanized antibody, remove a post-translational modification or for other reasons.

The term “monoclonal antibody” as used herein refers to an antibody obtained from a population of substantially homogeneous antibodies. i.e., the individual antibodies comprising the population are identical except for possible naturally occurring mutations that may be present in minor amounts. Monoclonal antibodies are highly specific, being directed against a single antigenic site. The modifier “monoclonal” is not to be construed as requiring production of the antibody by any particular method.

An “intact antibody” is one that comprises an antigen-binding variable region as well as a light chain constant domain (CL) and heavy chain constant domains, CH₁, CH₂, CH₃, and CH₄, as appropriate for the antibody class. The constant domains may be native sequence constant domains (e.g., human native sequence constant domains) or amino acid sequence variant thereof.

An “antibody fragment” comprises a portion of an intact antibody, comprising the antigen-binding or variable region thereof. Examples of antibody fragments include Fab, Fab′, F(ab′)₂, and Fv fragments, diabodies, triabodies, tetrabodies, linear antibodies, single-chain antibody molecules, scFv, scFv-Fe, multispecific antibody fragments formed from antibody fragment(s), a fragment(s) produced by a Fab expression library, or an epitope-binding fragment of any of the above which immunospecifically binds to a target antigen (e.g., a cancer cell antigen, a viral antigen or a microbial antigen).

An “antigen” is an entity to which an antibody specifically binds.

The terms “specific binding” and “specifically binds” mean that the antibody or antibody derivative will bind, in a highly selective manner, to its corresponding target antigen and not with the multitude of other antigens. Typically, the antibody or antibody derivative binds with an affinity of at least about 1×10⁻⁷ M, 10⁻⁸ M, 10⁻⁹M, 10⁻¹⁰ M, 10⁻¹¹ M, or 10⁻¹² M and binds to the predetermined antigen with an affinity that is at least two-fold greater than its affinity for binding to a non-specific antigen (e.g., BSA, casein) other than the predetermined antigen or a closely related antigen.

The term “inhibit” or “inhibition of” means to reduce by a measurable amount, or to prevent entirely.

The term “therapeutically effective amount” refers to an amount of a drug effective to treat a disease or disorder in a mammal. In the case of cancer, the therapeutically effective amount of a drug may reduce the number of cancer cells; reduce the tumor size; inhibit (i.e., slow to some extent or stop) cancer cell infiltration into peripheral organs; inhibit (i.e., slow to some extent or stop) tumor metastasis; inhibit, to some extent, tumor growth; and/or relieve to some extent one or more of the symptoms associated with the cancer. To the extent the drug may inhibit growth and/or kill existing cancer cells, it may be cytostatic and/or cytotoxic. For cancer therapy, efficacy can, for example, be measured by assessing the time to disease progression (TTP) and/or determining the response rate (RR).

The term “substantial” or “substantially” refers to a majority, i.e. >50% of a population, of a mixture or a sample, preferably more than 50%, 55%, 60%, 65%, 70%, 75%, 80%, 85%, 90%, 91%, 92%, 93%, 94%, 95%, 96%, 97%, 98%, or 99% of a population.

The terms “intracellularly cleaved” and “intracellular cleavage” refer to a metabolic process or reaction inside a cell on a ligand drug conjugate (e.g., an antibody drug conjugate (ADC)), whereby the covalent attachment, e.g., the linker, between the drug moiety (D) and the ligand unit (e.g., an antibody (BA or Ab)) is broken, resulting in the free drug, or another metabolite of the conjugate dissociated from the antibody inside the cell. The cleaved moieties of the drug-linker-ligand conjugate are thus intracellular metabolites.

The term “cytotoxic activity” refers to a cell-killing, a cytostatic or an anti-proliferative effect of a drug-linker-ligand conjugate compound or an intracellular metabolite of a drug-linker-ligand conjugate. Cytotoxic activity may be expressed as the IC50 value, which is the concentration (molar or mass) per unit volume at which half the cells survive.

The term “cytotoxic agent” as used herein refers to a substance that inhibits the function of cells and/or causes destruction of cells. The term is intended to include radioactive isotopes (e.g., 211At, 131I, 125I, 90Y, 186Re, 188Re, 153Sm, 212Bi, 32P, 60C, and radioactive isotopes of Lu), chemotherapeutic agents, and toxins such as small molecule toxins or enzymatically active toxins of bacterial, fungal, plant, or animal origin, including synthetic analogs and derivatives thereof.

The terms “cancer” and “cancerous” refer to or describe the physiological condition or disorder in mammals that is typically characterized by unregulated cell growth. A “tumor” comprises one or more cancerous cells.

An “autoimmune disease” herein is a disease or disorder arising from and directed against an individual's own tissues or proteins.

Examples of a “patient” or “subject” include, but are not limited to, mammals such as a human, rat, mouse, guinea pig, monkey, pig, goat, cow, horse, dog, or cat, and birds or fowl. In an embodiment, the patient is a human.

The terms “treat” or “treatment,” unless otherwise indicated by context, refer to therapeutic treatment and prophylactic measures to prevent relapse, wherein the object is to inhibit or slow down (lessen) an undesired physiological change or disorder, such as the development or spread of cancer. For purposes of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder.

In the context of cancer, the term “treating” includes any or all of inhibiting growth of tumor cells, cancer cells, or of a tumor, inhibiting replication of tumor cells or cancer cells, lessening of overall tumor burden or decreasing the number of cancerous cells, and ameliorating one or more symptoms associated with the disease.

In the context of an autoimmune disease, the term “treating” includes any or all of inhibiting replication of cells associated with an autoimmune disease state including, but not limited to, cells that produce an autoimmune antibody, lessening the autoimmune-antibody burden, and ameliorating one or more symptoms of an autoimmune disease.

As used herein, and in the specification and the accompanying claims, the indefinite articles “a” and “an” and the definite article “the” include the plural as well as single referents, unless the context clearly indicates otherwise.

As used herein, and unless otherwise specified, the terms “about” and “approximately,” when used in connection with amounts, or weight percentage of ingredients of a composition, mean an amount or weight percent that is recognized by one of ordinary skill in the art to provide a pharmacological effect equivalent to that obtained from the specified amount or weight percent. In certain embodiments, the terms “about” and “approximately,” when used in this context, contemplate an amount or weight percent within 30%, within 20%, within 15%, within 10%, or within 5%, of the specified amount or weight percent.

As used herein, and unless otherwise specified, the terms “about” and “approximately,” when used in connection with a numeric value or range of values that is provided to characterize a particular solid form, e.g., a specific temperature or temperature range, such as, for example, that describes a melting, dehydration, desolvation, or glass transition temperature; a mass change, such as, for example, a mass change as a function of temperature or humidity; a solvent or water content, in terms of, for example, mass or a percentage; or a peak position, such as, for example, in analysis by, for example, IR or Raman spectroscopy or XRPD; indicate that the value or range of values may deviate to an extent deemed reasonable to one of ordinary skill in the art while still describing the solid form. Techniques for characterizing crystal forms and amorphous solids include, but are not limited to, thermal gravimetric analysis (TGA), differential scanning calorimetry (DSC), X-ray powder diffractometry (XRPD), single-crystal X-ray diffractometry, vibrational spectroscopy, e.g., infrared (IR) and Raman spectroscopy, solid-state and solution nuclear magnetic resonance (NMR) spectroscopy, optical microscopy, hot stage optical microscopy, scanning electron microscopy (SEM), electron crystallography and quantitative analysis, particle size analysis (PSA), surface area analysis, solubility studies, and dissolution studies. In certain embodiments, the terms “about” and “approximately,” when used in this context, indicate that the numeric value or range of values may vary within 30%, 20%, 15%, 10%, 9%, 8%, 7%, 6%, 5%, 4%, 3%, 2%, 1.5%, 1%, 0.5%, or 0.25% of the recited value or range of values. For example, in some embodiments, the value of an XRPD peak position may vary by up to ±0.2° 2θ while still describing the particular XRPD peak.

As used herein, the term “inclusive,” when used in reference to range, includes the endpoints of the range. For example, if n is an integer from 0 to 4, n may be any of 0, 1, 2, 3, or 4.

An “alkyl” group is a saturated, partially saturated, or unsaturated straight chain or branched non-cyclic hydrocarbon having from 1 to 10 carbon atoms, typically from 1 to 8 carbons or, in some embodiments, from 1 to 6, 1 to 4, or 2 to 6 carbon atoms. Representative alkyl groups include -methyl, -ethyl, -n-propyl, -n-butyl, -n-pentyl, and n-hexyl; saturated branched alkyls include -isopropyl, -sec-butyl, -isobutyl, -tert-butyl, -isopentyl, 2-methylpentyl, 3-methylpentyl, 4-methylpentyl, 2,3-dimethylbutyl and the like. Examples of unsaturated alkyl groups include, but are not limited to, vinyl, allyl, CH═CH(CH₃), —CH═C(CH₃)₂, —C(CH₃)═CH₂, —C(CH₃)═CH(CH₃), C(CH₂CH₃)═CH₂, C≡CH, —C≡C(CH₃), —C≡C(CH₂CH₃), —CH₂C≡CH, —CH₂C≡C(CH₃), and CH₂C≡C(CH₂CH₃), among others. An alkyl group can be substituted or unsubstituted. In certain embodiments, when the alkyl groups described herein are said to be “substituted,” they may be substituted with any substituent or substituents as those found in the compounds and embodiments disclosed herein, as well as halogen (chloro, iodo, bromo, or fluoro); hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂; or O(alkyl)aminocarbonyl.

An “alkenyl” group is a straight chain or branched non-cyclic hydrocarbon having from 2 to 10 carbon atoms, typically from 2 to 8 carbon atoms, and including at least one carbon-carbon double bond. Representative straight chain and branched (C₂-C₈)alkenyls include -vinyl, -allyl, -1-butenyl, -2-butenyl, -isobutylenyl, -1-pentenyl, -2-pentenyl, -3-methyl-1-butenyl, -2-methyl-2-butenyl, -23-dimethyl-2-butenyl, -1-hexenyl, 2-hexenyl, -3-hexenyl, -1-heptenyl, -2-heptenyl, -3-heptenyl, -1-octenyl, -2-octenyl, 3-octenyl and the like. The double bond of an alkenyl group can be unconjugated or conjugated to another unsaturated group. An alkenyl group can be unsubstituted or substituted.

A “cycloalkyl” group is a saturated or a partially saturated cyclic alkyl group of from 3 to 10 carbon atoms having a single cyclic ring or multiple condensed or bridged rings which can be optionally substituted with from 1 to 3 alkyl groups. In some embodiments, the cycloalkyl group has 3 to 8 ring members, whereas in other embodiments the number of ring carbon atoms ranges from 3 to 5, 3 to 6, or 3 to 7. Such cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as adamantyl and the like. Examples of unsaturated cycloalkyl groups include cyclohexenyl, cyclopentenyl, cyclohexadienyl, butadienyl, pentadienyl, and hexadienyl, among others. A cycloalkyl group can be substituted or unsubstituted. Such substituted cycloalkyl groups include, by way of example, cyclohexanone and the like.

An “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6 to 14 carbons, and in others from 6 to 12 or even 6 to 10 carbon atoms in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

An “arylene” group is a bivalent aryl group as defined herein.

A “heteroaryl” group is an aryl ring system having one to four heteroatoms as ring atoms in a heteroaromatic ring system, wherein the remainder of the atoms are carbon atoms. In some embodiments, heteroaryl groups contain 5 to 6 ring atoms, and in others from 6 to 9 or 6 to 10 atoms in the ring portions of the groups. Suitable heteroatoms include oxygen, sulfur, and nitrogen. In certain embodiments, the heteroaryl ring system is monocyclic or bicyclic. Non-limiting examples include, but are not limited to, groups such as pyrrolyl, pyrazolyl, imidazolyl, triazolyl, tetrazolyl, oxazolyl, isoxazolyl, thiazolyl, pyrrolyl, pyridyl, pyridazinyl, pyrimidinyl, pyrazinyl, thiophenyl, benzothiophenyl, furanyl, benzofuranyl (for example, isobenzofuran-1,3-diimine), indolyl, azaindolyl (for example, pyrrolopyridyl or 1H-pyrrolo[2,3-b]pyridyl), indazolyl, benzimidazolyl (for example, 1H-benzo[d]imidazolyl), imidazopyridyl (for example, azabenzimidazolyl, 3H-imidazo[4,5-b]pyridyl or 1H-imidazo[4,5-b]pyridyl), pyrazolopyridyl, triazolopyridyl, benzotriazolyl, benzoxazolyl, benzothiazolyl, benzothiadiazolyl, isoxazolopyridyl, thianaphthalenyl, purinyl, xanthinyl, adeninyl, guaninyl, quinolinyl, isoquinolinyl, tetrahydroquinolinyl, quinoxalinyl, and quinazolinyl groups.

A “heteroarylene” group is a bivalent heteroaryl group as defined herein.

A “heterocyclyl” is an aromatic (also referred to as heteroaryl) or non-aromatic cycloalkyl in which one to four of the ring carbon atoms are independently replaced with a heteroatom from the group consisting of O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclyl group can be substituted or unsubstituted. Heterocyclyl groups encompass unsaturated, partially saturated, and saturated ring systems, such as, for example, imidazolyl, imidazolinyl, and imidazolidinyl groups. The term “heterocyclyl” includes fused ring species, including those comprising fused aromatic and non-aromatic groups, such as, for example, benzotriazolyl, 2,3-dihydrobenzo[1,4]dioxinyl, and benzo[1,3]dioxolyl. The term also includes bridged polycyclic ring systems containing a heteroatom such as, but not limited to, quinuclidyl. Representative examples of a heterocyclyl group include, but are not limited to, aziridinyl, azetidinyl, pyrrolidyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl (for example, tetrahydro-2H-pyranyl), tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl; for example, 1H-imidazo[4,5-b]pyridyl, or 1H-imidazo[4,5-b]pyridyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

A “cycloalkylalkyl” group is a radical of the formula -alkyl-cycloalkyl, wherein alkyl and cycloalkyl are defined above. Substituted cycloalkylalkyl groups may be substituted at the alkyl, the cycloalkyl, or both the alkyl and the cycloalkyl portions of the group. Representative cycloalkylalkyl groups include but are not limited to cyclopentylmethyl, cyclopentylethyl, cyclohexylmethyl, cyclohexylethyl, and cyclohexylpropyl. Representative substituted cycloalkylalkyl groups may be mono-substituted or substituted more than once.

An “aralkyl” group is a radical of the formula -alkyl-aryl, wherein alkyl and aryl are defined above. Substituted aralkyl groups may be substituted at the alkyl, the aryl, or both the alkyl and the aryl portions of the group. Representative aralkyl groups include, but are not limited to, benzyl and phenethyl groups and fused (cycloalkylaryl)alkyl groups such as 4-ethyl-indanyl.

A “heterocyclylalkyl” group is a radical of the formula -alkyl-heterocyclyl, wherein alkyl and heterocyclyl are defined above. Substituted heterocyclylalkyl groups may be substituted at the alkyl, the heterocyclyl, or both the alkyl and the heterocyclyl portions of the group. Representative heterocyclylalkyl groups include, but are not limited to, 4-ethyl-morpholinyl, 4-propylmorpholinyl, furan-2-yl methyl, furan-3-yl methyl, pyrdine-3-yl methyl, (tetrahydro-2H-pyran-4-yl)methyl, (tetrahydro-2H-pyran-4-yl)ethyl, tetrahydrofuran-2-yl methyl, tetrahydrofuran-2-yl ethyl, and indol-2-yl propyl.

A “halogen” is chloro, iodo, bromo, or fluoro.

A “hydroxyalkyl” group is an alkyl group as described above substituted with one or more hydroxy groups.

An “alkoxy” group is O(alkyl), wherein alkyl is defined above.

An “alkoxyalkyl” group is (alkyl)O(alkyl), wherein alkyl is defined above.

As used herein, “alkynyl” refers to a monovalent hydrocarbon radical moiety containing at least two carbon atoms and one or more carbon-carbon triple bonds. Alkynyl is optionally substituted and can be linear, branched, or cyclic. Alkynyl includes, but is not limited to, those radicals having 2-20 carbon atoms, i.e., C₂₋₂₀ alkynyl; 2-12 carbon atoms, i.e., C_{2_12} alkynyl; 2-8 carbon atoms, i.e., C₂₋₈ alkynyl; 2-6 carbon atoms, i.e., C₂₋₆ alkynyl; and 2-4 carbon atoms, i.e., C₂₋₄ alkynyl. Examples of alkynyl moieties include, but are not limited to ethynyl, propynyl, and butynyl.

As used herein, “haloalkyl” refers to alkyl, as defined above, wherein the alkyl includes at least one substituent selected from a halogen, for example, fluorine (F), chlorine (Cl), bromine (Br), or iodine (I). Examples of haloalkyl include, but are not limited to, —CF₃, —CH₂CF₃, —CCl₂F, and —CCl₃.

As used herein, “haloalkoxy” refers to alkoxy, as defined above, wherein the alkoxy includes at least one substituent selected from a halogen, e.g., F, Cl, Br, or I.

As used herein, “arylalkyl” refers to a monovalent moiety that is a radical of an alkyl compound, wherein the alkyl compound is substituted with an aromatic substituent, i.e., the aromatic compound includes a single bond to an alkyl group and wherein the radical is localized on the alkyl group. An arylalkyl group bonds to the illustrated chemical structure via the alkyl group. An arylalkyl can be represented by the structure, e.g., B—CH₂—, B—CH₂—CH₂—, B—CH₂—CH₂—CH₂—, B—CH₂—CH₂—CH₂—CH₂—, B—CH(CH₃)—CH₂—CH₂—, B—CH₂—CH(CH₃)—CH₂—, wherein B is an aromatic moiety, e.g., phenyl. Arylalkyl is optionally substituted, i.e., the aryl group and/or the alkyl group, can be substituted as disclosed herein. Examples of arylalkyl include, but are not limited to, benzyl.

As used herein, “alkylaryl” refers to a monovalent moiety that is a radical of an aryl compound, wherein the aryl compound is substituted with an alkyl substituent, i.e., the aryl compound includes a single bond to an alkyl group and wherein the radical is localized on the aryl group. An alkylaryl group bonds to the illustrated chemical structure via the aryl group. An alkylaryl can be represented by the structure, e.g., —B—CH₃, —B—CH₂—CH₃, —B—CH₂—CH₂—CH₃, —B—CH₂—CH₂—CH₂—CH₃, —B—CH(CH₃)—CH₂—CH₃, —B—CH₂—CH(CH₃)—CH₃, wherein B is an aromatic moiety, e.g., phenyl. Alkylaryl is optionally substituted, i.e., the aryl group and/or the alkyl group, can be substituted as disclosed herein. Examples of alkylaryl include, but are not limited to, toluyl.

As used herein, “aryloxy” refers to a monovalent moiety that is a radical of an aromatic compound wherein the ring atoms are carbon atoms and wherein the ring is substituted with an oxygen radical, i.e., the aromatic compound includes a single bond to an oxygen atom and wherein the radical is localized on the oxygen atom, e.g., C₆H₅—O—, for phenoxy. Aryloxy substituents bond to the compound which they substitute through this oxygen atom. Aryloxy is optionally substituted. Aryloxy includes, but is not limited to, those radicals having 6 to 20 ring carbon atoms, i.e., C₆₋₂₀ aryloxy; 6 to 15 ring carbon atoms, i.e., C₆₋₁₅ aryloxy, and 6 to 10 ring carbon atoms, i.e., C₆₋₁₀ aryloxy. Examples of aryloxy moieties include, but are not limited to phenoxy, naphthoxy, and anthroxy.

An “amino” group is a radical of the formula NH₂.

A “hydroxyl amine” group is a radical of the formula N(R^{#})OH or NHOH, wherein R^{#} is a substituted or unsubstituted alkyl, cycloalkyl, cycloalkylalkyl, aryl, aralkyl, heterocyclyl or heterocyclylalkyl group as defined herein.

An “alkoxyamine” group is a radical of the formula —N(R^{#})O-alkyl or —NHO-alkyl, wherein R^{#} is as defined above.

An “aralkoxyamine” group is a radical of the formula N(R^{#})O-aryl or NHOaryl, wherein R^{#} is as defined above.

An “alkylamine” group is a radical of the formula NHalkyl or N(alkyl)₂, wherein each alkyl is independently as defined above.

An “aminocarbonyl” group is a radical of the formula —C(═O)N(R^{#})₂, —C(═O)NH(R^{#}), or C(═O)NH₂, wherein each R^{#} is as defined above.

An “acylamino” group is a radical of the formula NHC(═O)(R^{#}) or N(alkyl)C(═O)(R^{#}), wherein each alkyl and R^{#} are independently as defined above.

An “O(alkyl)aminocarbonyl” group is a radical of the formula —O(alkyl)C(═O)N(R^{#})₂, —O(alkyl)C(═O)NH(R^{#}), or —O(alkyl)C(═O)NH₂, wherein each R^{#} is independently as defined above.

An “N-oxide” group is a radical of the formula —N⁺—O⁻.

A “carboxy” group is a radical of the formula C(═O)OH.

A “ketone” group is a radical of the formula C(═O)(R^{#}), wherein R^{#} is as defined above.

An “aldehyde” group is a radical of the formula —CH(═O).

An “ester” group is a radical of the formula C(═O)O(R^{#}) or OC(═O)(R^{#}), wherein R^{#} is as defined above.

A “urea” group is a radical of the formula —N(alkyl)C(═O)N(R^{#})₂, —N(alkyl)C(═O)NH(R^{#}), —N(alkyl)C(═O)NH₂, —NHC(═O)N(R^{#})₂, —NHC(═O)NH(R^{#}), or NHC(═O)NH₂^{#}, wherein each alkyl and R^{#} are independently as defined above.

An “imine” group is a radical of the formula —N═C(R^{#})₂ or —C(R^{#})═N(R^{#}), wherein each R^{#} is independently as defined above.

An “imide” group is a radical of the formula —C(═O)N(R^{#})C(═O)(R^{#}) or N((C═O)(R^{#}))₂, wherein each R^{#} is independently as defined above.

A “urethane” group is a radical of the formula —OC(═O)N(R^{#})₂, —OC(═O)NH(R^{#}), —N(R^{#})C(═O)O(R^{#}), or —NHC(═O)O(R^{#}), wherein each R^{#} is independently as defined above.

An “amidine” group is a radical of the formula —C(═N(R^{#}))N(R^{#})₂, —C(═N(R^{#}))NH(R^{#}), —C(═N(R^{#}))NH₂, —C(═NH)N(R^{#})₂, —C(═NH)NH(R^{#}), —C(═NH)NH₂, —N═C(R^{#})N(R^{#})₂, —N═C(R^{#})NH(R^{#}), —N═C(R^{#})NH₂, —N(R^{#})C(R^{#})═N(R^{#}), —NHC(R^{#})═N(R^{#}), —N(R^{#})C(R^{#})═NH, or —NHC(R^{#})═NH, wherein each R^{#} is independently as defined above.

A “guanidine” group is a radical of the formula —N(R^{#})C(═N(R^{#}))N(R^{#})₂, —NHC(═N(R^{#}))N(R^{#})₂, —N(R^{#})C(═NH)N(R^{#})₂, —N(R^{#})C(═N(R^{#}))NH(R^{#}), —N(R^{#})C(═N(R^{#}))NH₂, —NHC(═NH)N(R^{#})₂, —NHC(═N(R^{#}))NH(R^{#}), —NHC(═N(R^{#}))NH₂, —NHC(═NH)NH(R^{#}), —NHC(═NH)NH₂, —N═C(N(R^{#})₂)₂, —N═C(NH(R^{#}))₂, or —N═C(NH₂)₂, wherein each R^{#} is independently as defined above.

An “enamnine” group is a radical of the formula —N(R^{#})C(R^{#})═C(R^{#})₂, —NHC(R^{#})═C(R^{#})₂, —C(N(R^{#})₂)═C(R^{#})₂, —C(NH(R^{#}))═C(R^{#})₂, —C(NH₂)═C(R^{#})₂, —C(R^{#})═C(R^{#})(N(R^{#})), C(R^{#})═C(RV)(NH(R^{#})) or —C(R^{#})═C(R^{#})(NH₂), wherein each R^{#} is independently as defined above.

An “oxime” group is a radical of the formula —C(═NO(R^{#}))(R^{#}), —C(═NOH)(R^{#}), —CH(═NO(R^{#})), or —CH(═NOH), wherein each R^{#} is independently as defined above.

A “hydrazide” group is a radical of the formula —C(═O)N(R^{#})N(R^{#})₂, —C(═O)NHN(R^{#})₂, —C(═O)N(R^{#})NH(R^{#}), —C(═O)N(R^{#})NH₂, —C(═O)NHNH(R^{#})₂, or —C(═O)NHNH₂, wherein each R^{#} is independently as defined above.

A “hydrazine” group is a radical of the formula —N(R^{#})N(R^{#})₂, —NH—N(R^{#})₂, —N(R^{#})NH(R^{#}), —N(R^{#})NH₂, —NHNH(R^{#})₂, or —NHNH₂, wherein each R^{#} is independently as defined above.

A “hydrazone” group is a radical of the formula —C(═N—N(R^{#})₂)(R^{#})₂, —C(═NNH(R^{#}))(R^{#})₂, —C(═N—NH₂)(R^{#})₂, —N(R^{#})(N═C(R^{#})₂), or —NH(N═C(R^{#})), wherein each R is independently as defined above.

An “azide” group is a radical of the formula —N₃.

An “isocyanate” group is a radical of the formula N═C═O.

An “isothiocyanate” group is a radical of the formula N═C═S.

A “cyanate” group is a radical of the formula OCN.

A “thiocyanate” group is a radical of the formula SCN.

A “thioether” group is a radical of the formula —S(R^{#}), wherein R^{#} is as defined above.

A “thiocarbonyl” group is a radical of the formula —C(═S)(R^{#}), wherein R^{#} is as defined above.

A “sulfinyl” group is a radical of the formula —S(═O)(R^{#}), wherein R^{#} is as defined above.

A “sulfone” group is a radical of the formula —S(═O)₂(R^{#}), wherein R^{#} is as defined above.

A “sulfonylamino” group is a radical of the formula —NHSO₂(R^{#}) or —N(alkyl)SO₂(R^{#}), wherein each alkyl and R^{#} are defined above.

A “sulfonamide” group is a radical of the formula —S(═O)₂N(R^{#})₂, or —S(═P)₂NH(R^{#}), or —S(═O)₂NH₂, wherein each R is independently as defined above.

A “phosphonate” group is a radical of the formula —P(═O)(O(R^{#}))₂, —P(═O)(OH)₂, —OP(═P)(O(R^{#}))(R^{#}), or —OP(═O)(OH)(R^{#}), wherein each R^{#} is independently as defined above.

A “phosphine” group is a radical of the formula —P(R^{#})₂, wherein each R^{#} is independently as defined above.

When the groups described herein, with the exception of alkyl groups, are said to be “substituted,” they may be substituted with any appropriate substituent or substituents. Illustrative examples of substituents are those found in the compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothiophenyl, or benzofuranyl) aryloxy; aralkyloxy; heterocyclyloxy; and heterocyclyl alkoxy.

As used herein, the term “pharmaceutically acceptable salt(s)” refers to a salt prepared from a pharmaceutically acceptable non-toxic acid or base including an inorganic acid or base and an organic acid or base.

As used herein and unless otherwise indicated, the term “solvate” means a compound, or a salt thereof, that further includes a stoichiometric or non-stoichiometric amount of a solvent bound by non-covalent intermolecular forces. In one embodiment, the solvate is a hydrate.

As used herein and unless otherwise indicated, the term “hydrate” means a compound, or a salt thereof, that further includes a stoichiometric or non-stoichiometric amount of water bound by non-covalent intermolecular forces.

As used herein and unless otherwise indicated, the term “prodrug” means a compound derivative that can hydrolyze, oxidize, or otherwise react under biological conditions (in vitro or in vivo) to provide an active compound. Examples of prodrugs include, but are not limited to, derivatives and metabolites of a compound that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. In certain embodiments, prodrugs of compounds with carboxyl functional groups are the lower alkyl esters of the carboxylic acid. The carboxylate esters may be formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh).

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments disclosed herein, including mixtures thereof. The use of stereomerically pure forms of such compounds, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (WileyInterscience, New York, 1981); Wilen, S. H., et al., Tetrahedron 33:2725 (1977); Eliel, E. L., Stereochemistry of Carbon Compounds (McGrawHill, NY, 1962); and Wilen, S. H., Tables of Resolving Agents and Optical Resolutions p. 268 (E. L. Eliel, Ed., Univ. of Notre Dame Press, Notre Dame, IN, 1972).

It should also be noted that the compounds can include E and Z isomers, or a mixture thereof, and cis and trans isomers, or a mixture thereof. In certain embodiments, the compounds are isolated as either the cis or trans isomer. In other embodiments, the compounds are a mixture of the cis and trans isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in an aqueous solution, pyrazoles may exhibit the following isomeric forms, which are referred to as tautomers of each other:

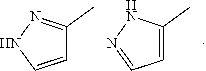

As readily understood by one skilled in the art, a wide variety of functional groups and other structures may exhibit tautomerism and all tautomers of the compounds are within the scope of the present disclosure.

It should also be noted the compounds can contain unnatural proportions of atomic isotopes at one or more of the atoms. For example, the compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with deuterium (²H), carbon-13 (¹³C), or nitrogen-5 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds, for example, the isotopologues are deuterium, carbon-13, or nitrogen-15 enriched compounds.

It should be noted that if there is a discrepancy between a depicted structure and a name for that structure, the depicted structure is to be accorded more weight.

As used herein, the term “residue” refers to the chemical moiety within a compound that remains after a chemical reaction. For example, the term “amino acid residue” or “N-alkyl amino acid residue” refers to the product of an amide coupling or peptide coupling of an amino acid or a N-alkyl amino acid to a suitable coupling partner; wherein, for example, a water molecule is expelled after the amide or peptide coupling of the amino acid or the N-alkylamino acid, resulting in the product having the amino acid residue or N-alkyl amino acid residue incorporated therein.

As used herein, “sugar” or “sugar group” or “sugar residue” refers to a carbohydrate moiety which may comprise 3-carbon (those) units, 4-carbon (tetrose) units, 5-carbon (pentose) units, 6-carbon (hexose) units, 7-carbon (heptose) units, or combinations thereof, and may be a monosaccharide, a disaccharide, a trisaccharide, a tetrasaccharide, a pentasaccharide, an oligosaccharide, or any other polysaccharide. In some instances, a “sugar” or “sugar group” or “sugar residue” comprises furanoses (e.g., ribofuranose, fructofuranose) or pyranoses (e.g., glucopyranose, galactopyranose), or a combination thereof. In some instances, a “sugar” or “sugar group” or “sugar residue” comprises aldoses or ketoses, or a combination thereof. Non-limiting examples of monosaccharides include ribose, deoxyribose, xylose, arabinose, glucose, mannose, galactose, and fructose. Non-limiting examples of disaccharides include sucrose, maltose, lactose, lactulose, and trehalose. Other “sugars” or “sugar groups” or “sugar residues” include polysaccharides and/or oligosaccharides, including, but not limited to, amylose, amylopectin, glycogen, inulin, and cellulose. In some instances, a “sugar” or “sugar group” or “sugar residue” is an amino-sugar. In some instances, a “sugar” or “sugar group” or “sugar residue” is a glucamine residue (1-amino-1-deoxy-D-glucitol) linked to the rest of molecule via its amino group to form an amide linkage with the rest of the molecule (i.e., a glucamide).

As used herein, “binding agent” refers to any molecule, e.g., antibody, capable of binding with specificity to a given binding partner, e.g., antigen.

As used herein, the term “amino acid” refers to an organic compound that contains amino (—NH₂) and carboxyl (—COOH) functional groups, along with a side chain (R group), which is specific to each amino acid. Amino acids may be proteinogenic or non-proteinogenic. By “proteinogenic” is meant that the amino acid is one of the twenty naturally occurring amino acids found in proteins. The proteinogenic amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. By “non-proteinogenic” is meant that either the amino acid is not found naturally in protein or is not directly produced by cellular machinery (e.g., is the product of post-translational modification). Non-limiting examples of non-proteinogenic amino acids include gamma-aminobutyric acid (GABA), taurine (2-aminoethanesulfonic acid), theanine (L-γ-glutamylethylamide), hydroxyproline, beta-alanine, ornithine, and citrulline.

As used herein “peptide,” in its various grammatical forms, is defined in its broadest sense to refer to a compound of two or more subunit amino acids, amino acid analogs, or other peptidomimnetics. The subunits may be linked by peptide bonds or by other bonds, for example, ester, ether, and the like. As used herein, the term “amino acid” refers to either natural and/or unnatural, proteinogenic or non-proteinogenic, or synthetic amino acids, including glycine and both the D and L optical isomers, and amino acid analogs and peptidomimetics. If the peptide chain is short, e.g., two, three or more amino acids, it is commonly called an oligopeptide. If the peptide chain is longer, the peptide is typically called a polypeptide or a protein. Full-length proteins, analogs, mutants, and fragments thereof are encompassed by the definition. The terms also include post-expression modifications of the polypeptide, for example, glycosylation, acetylation, phosphorylation, and the like. Furthermore, as ionizable amino and carboxyl groups are present in the molecule, a particular peptide may be obtained as an acidic or basic salt, or in neutral form. A peptide may be obtained directly from the source organism or may be recombinantly or synthetically produced.

The amino acid sequence of an antibody can be numbered using any known numbering schemes, including those described by Kabat et al., (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Pluckthun, J. Mol. Biol., 2001, 309:657-70 (“AHo” numbering scheme). Unless otherwise specified, the numbering scheme used herein is the Kabat numbering scheme. However, selection of a numbering scheme is not intended to imply differences in sequences where they do not exist, and one of skill in the art can readily confirm a sequence position by examining the amino acid sequence of one or more antibodies. Unless stated otherwise, the “EU numbering scheme” is generally used when referring to a residue in an antibody heavy chain constant region (e.g., as reported in Kabat et al., supra).

As used herein, the term “anti-HER2 antibody” refers to an antibody selectively binding to the HER2 receptor, e.g., trastuzumab. In one embodiment, trastuzumab can be made and used as described in U.S. Pat. Nos. 6,407,213 and 5,821,337, the entire disclosures of which are incorporated herein by reference.

As used herein, the term “anti-HER3 antibody” refers to an antibody selectively binding to the HER3 receptor, e.g., patritumab. In one embodiment, patritumab can be made and used as described in U.S. Pat. No. 7,705,130, the entire disclosure of which is incorporated herein by reference.

As used herein, the term “anti-PTK7 antibody” refers to an antibody selectively binding to the PTK7 receptor, e.g., cofetuzumab. In one embodiment, cofetuzumab can be made and used as described in U.S. Pat. No. 9,777,070, the entire disclosure of which is incorporated herein by reference.

As used herein, the term “ifinatamab” refers to an antibody selectively binding to the B7H3 receptor, i.e., an anti-human B7H3 antibody. In one embodiment, ifinatamab can be made and used as described in U.S. Ser. No. 10/117,952 or WO2022102695, the entire disclosures of which are incorporated herein by reference.

As used herein, the term “6E7” refers to a CLL1 monoclonal antibody.

As used herein, the term “cell-killing activity” refers to the activity that decreases or reduces the cell viability of the tested cell line.

In the claims that follow and in the preceding description, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments.

### Conjugates

In embodiments, a conjugate, or a pharmaceutically acceptable salt, tautomer, solvate, or stereoisomer thereof, includes a protein linked to at least one payload or payload residue (also referred to herein as a Drug unit) and linked to at least one hydrophilic moiety (also referred to herein as a cleavable unit or Cleavable) via a covalent linker. The covalent linker is bonded directly or indirectly to each of the protein, the payload residue, and the hydrophilic moiety. In some embodiments, the protein is a binding agent, such as an antibody or antigen-binding fragment thereof.

In some embodiments, the protein is bonded directly to a covalent linker, such as a linker set forth herein. In such cases, the binding agent is one bond position away from the covalent linker. The covalent linker may also be bonded directly to a payload residue such that the covalent linker is one bond position away from a payload residue. The payload may be any payload set forth herein, such as MMAE. In some embodiments, the covalent linker is also bonded directly to a hydrophilic moiety such that the covalent linker is one bond position away from a hydrophilic moiety. The hydrophilic moiety may be any hydrophilic moiety set forth herein.

In some embodiments, the binding agent is bonded indirectly to a covalent linker such that the binding agent is more than one bond position away from the covalent linker. In such cases, the binding agent is bonded through another moiety to the covalent linker. For example, the binding agent may be bonded to a maleimide group which is bonded to a polyethylene glycol group which is bonded to the covalent linker.

In some examples, the covalent linker is also bonded indirectly to a payload residue such that the covalent linker is more than one bond position away from a payload residue. The covalent linker is bonded through another moiety to the payload. For example, the covalent linker may be bonded to a dipeptide, such as but not limited to Val-Ala or Val-Cit, which may be bonded to a p-aminobenzyl alcohol (PAB)-derived unit, which may be bonded to the payload residue. An example of a PAB-derived unit is —NH—(C6H4)-CH2-O—C(═O)—, and the phenylene portion of the PAB unit may be substituted with —C1-C8 alkyl, —O—(C1-C8 alkyl), -halogen, -nitro, or -cyano.

In some embodiments, the covalent linker is bonded indirectly to a hydrophilic moiety such that the covalent linker is more than one bond position away from a hydrophilic moiety. The covalent linker is bonded through another moiety to the hydrophilic moiety.

The compounds disclosed herein include at least a Conjugator. Spacer, Cleavable, and Payload. In some embodiments, compounds include a Brancher. In some embodiments, such as of conjugates, compounds include a binding agent (BA). Conjugator is a group capable of bonding a BA and joining the BA to the rest of the compound. Cleavable includes at least one site that can be cleaved by a β-glucuronidase enzyme, such as a glycosidic linkage or bond. Cleavable may include a hydrophilic moiety. Spacer is a group that lengthens the linker and provides more distance between Conjugator and Payload. Spacer may join Conjugator to Cleavable or Conjugator to Brancher, if present. Brancher is a group that extends the framework of the linker and includes a hydrophile. Spacer, Cleavable, and Brancher may form the linker portion of a conjugate. Payload (also referred to herein as a Drug unit) is a cytotoxic agent or residue thereof.

### Aspect 1

Provided herein are compounds having formula (I)

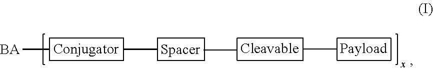

or a pharmaceutically acceptable salt, tautomer, solvate, or stereoisomer thereof, wherein:
BA is a binding agent selected from a humanized, monoclonal, chimeric, or human antibody or an antigen-binding fragment thereof;
Conjugator is selected from formula (II) and (III):

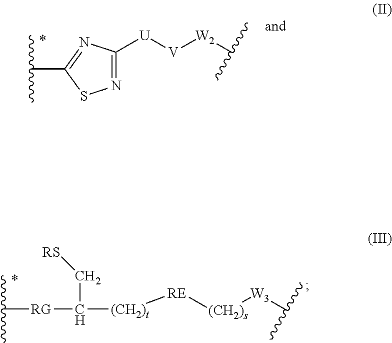

U is a bond, heteroarylene, or arylene;
V is a bond or —C≡C—(CH₂)ₙ—;
n is an integer from 0 to 10 inclusive;
W₂ is —C(═O)—, —NH—, or —O—;
RG is

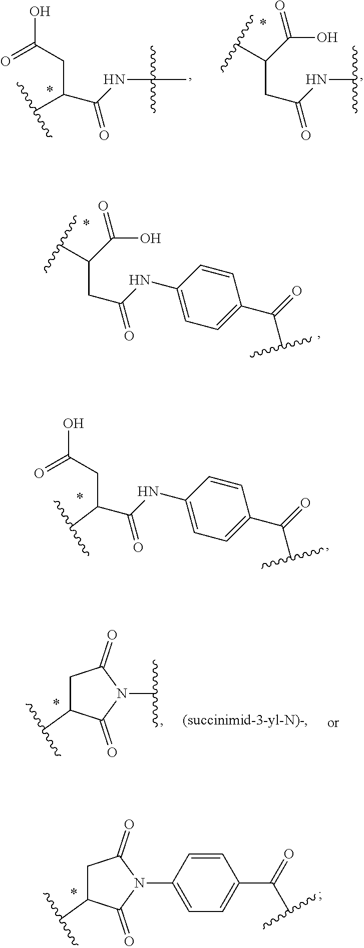 [SMILES: *NC(=O)C(*)CC(=O)O ; *NC(=O)CC(*)C(=O)O ; *C(=O)c1ccc(NC(=O)CC(*)C(=O)O)cc1 ; *C(=O)c1ccc(NC(=O)C(*)CC(=O)O)cc1 ; *C1CC(=O)N(*)C1=O ; *C(=O)c1ccc(N2C(=O)CC(*)C2=O)cc1]

RS is —NR^{1a}R^{1b};
each of R^{1a} and R^{1b} is, independently, H or substituted or unsubstituted C₁₋₄ alkyl;
RE is a bond, —O—, —OC(═O)—, —OC(═O)NR⁶—, —NHC(═O)NR⁶—, —OS(═O)₂NR⁶—, —NHS(═O)₂NR⁶—, —or —OC(═O)NHS(═O)₂NR⁶—;
R⁶ is H or substituted or unsubstituted C₁₋₄ alkyl;
W₃ is —C(═O)—, —NH—, or —O—;
each of s and t is, independently, 0, 1, or 2;
[symbol] indicates a point of covalent attachment within the compound;
* marks the bond where Conjugator connects to BA;
Spacer is a bond, **—NH—(CH₂CH₂O)ₘ—CH₂CH₂—C(═O)—, **—(CH₂)ₘ—C(═O)—, *—CH₂—C(═O)—NH—(CH₂)ₘ—C(═O)—, **—(CH₂CH₂O)ₘ—CH₂CH₂—C(═O)—, **—CH[—(CH₂)ₘ—COOH]—C(═O)—, **—CH₂—C(═O)—NH—(CH₂)ₘ—C(═O)—NH—(CH₂)ₘ—C(═O)—, **—C(═O)—(CH₂)ₘ—C(═O)—, **—C(═O)—(CH₂CH₂O)ₘ—CH₂CH₂—NH—, *—NH—(CH₂)ₘ—C(═O)—, or **—NH—(CH₂)ₘ—O—;
m is an integer from 1 to 12 inclusive;
** marks the bond where Spacer connects to Conjugator;
Cleavable has formula (IVa), (IVb), (IVc), (Va), (Vb), (VIa), (VIb), (VIIa), or (VIIb):

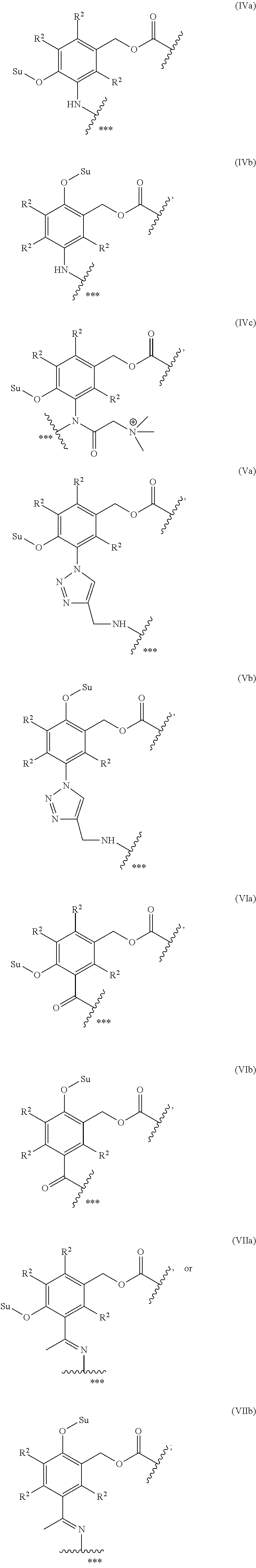

Su is a Sugar moiety;
each R² is, independently, hydrogen, halogen, substituted or unsubstituted C₁₋₄ alkyl, —CN, or —NO₂;
marks the bond where Cleavable connects to Spacer;
Payload is a payload residue; and
x is from 1 to 15 inclusive.

In some embodiments, Conjugator has formula (II).

In some embodiments, U is arylene.

In some embodiments, U is phenylene.

In some embodiments, U is

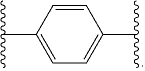 [SMILES: *c1ccc(*)cc1]

In some embodiments, U is arylene and V is a bond.

In some embodiments, U is arylene and V is —C≡C—(CH₂)ₙ—.

In some embodiments, U is arylene and V is —C≡C—(CH₂)₃—.

In some embodiments, U is heteroarylene.

In some embodiments, U is a bivalent pyrimidine ring.

In some embodiments, U is

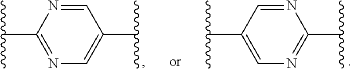 [SMILES: *c1cnc(*)nc1 ; *c1cnc(*)nc1]

In some embodiments, U is heteroarylene and V is a bond.

In some embodiments, U is heteroarylene and V is —C≡C—(CH₂)ₙ—.

In some embodiments, U is heteroarylene and V is —C≡C—(CH₂)₃—.

In some embodiments, U is a bond.

In some embodiments, U is a bond and V is a bond.

In some embodiments, U is a bond and V is —C≡C—(CH₂)ₙ—.

In some embodiments, U is a bond and V is —C≡C—(CH₂)₃—.

In some embodiments, W₂ is —C(═O)—.

In some embodiments, Conjugator has formula (III).

In some embodiments, RS is —NH₂ or —N(CH₃)₂.

In some embodiments, RS is —NH₂.

In some embodiments, RE is —OC(═O) NH—.

In some embodiments, RG is

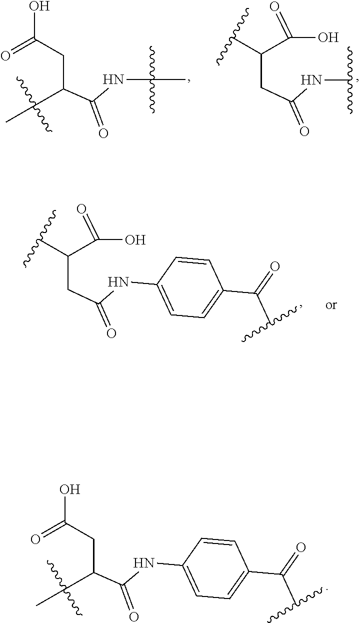 [SMILES: *NC(=O)C(*)CC(=O)O ; *NC(=O)CC(*)C(=O)O ; *C(=O)c1ccc(NC(=O)CC(*)C(=O)O)cc1 ; *C(=O)c1ccc(NC(=O)C(*)CC(=O)O)cc1]

In some embodiments, RG is

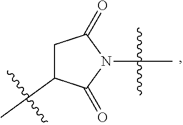 [SMILES: *C1CC(=O)N(*)C1=O]

-(succinimid-3-yl-N)—, or

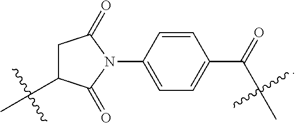 [SMILES: *C(=O)c1ccc(N2C(=O)CC(*)C2=O)cc1]

In some embodiments, RG is

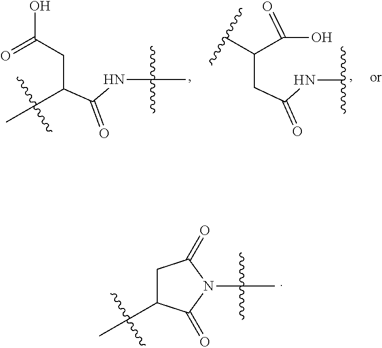 [SMILES: *NC(=O)C(*)CC(=O)O ; *NC(=O)CC(*)C(=O)O ; *C1CC(=O)N(*)C1=O]

Certain RG described herein, e.g.,

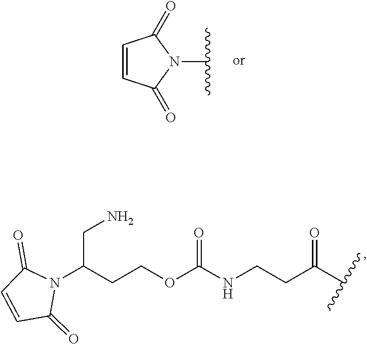 [SMILES: *N1C(=O)C=CC1=O ; *C(=O)CCNC(=O)OCCC(CN)N1C(=O)C=CC1=O]

may, upon conjugation with BA, be induced to undergo a ring opening process. During such process the maleimide structure formed upon conjugation of the RG with BA, e.g.,

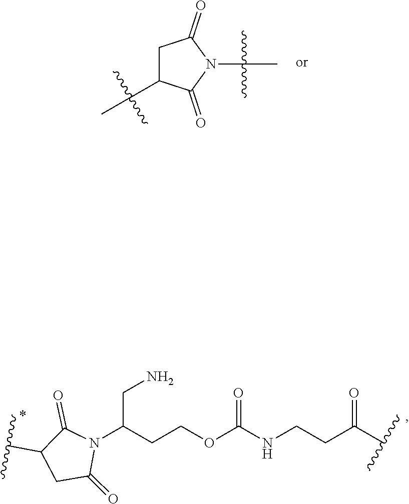 [SMILES: *C1CC(=O)N(*)C1=O ; *C(=O)CCNC(=O)OCCC(CN)N1C(=O)CC(*)C1=O]

undergoes ring opening. It will be appreciated that there are two possible regioisomers that may result from the ring opening of such maleimide structures. For example,

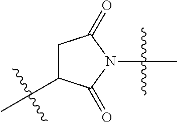 [SMILES: *C1CC(=O)N(*)C1=O]

may undergo ring opening to form

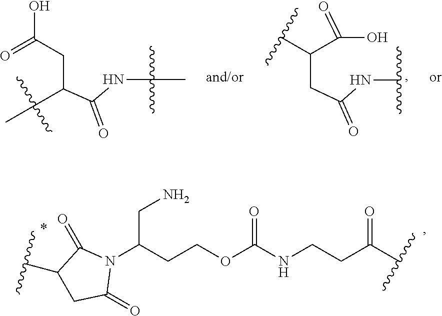 [SMILES: *NC(=O)C(*)CC(=O)O ; *NC(=O)CC(*)C(=O)O ; *C(=O)CCNC(=O)OCCC(CN)N1C(=O)CC(*)C1=O]

may undergo ring opening to form

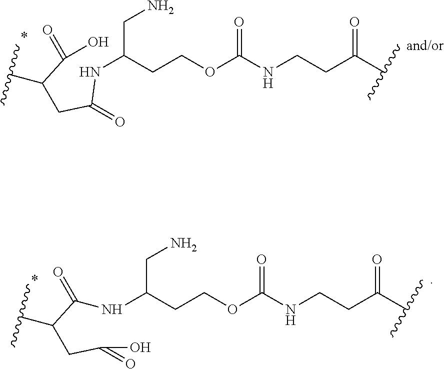 [SMILES: *C(=O)CCNC(=O)OCCC(CN)NC(=O)CC(*)C(=O)O ; *C(=O)CCNC(=O)OCCC(CN)NC(=O)C(*)CC(=O)O]

For simplicity, unless indicated otherwise, both possible ring-open regioisomers are encompassed by a depiction of a single ring-open regioisomer herein. Thus, unless indicated otherwise, depiction of the ring-open regioisomer

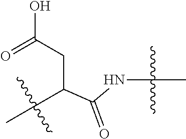 [SMILES: *NC(=O)C(*)CC(=O)O]

encompasses

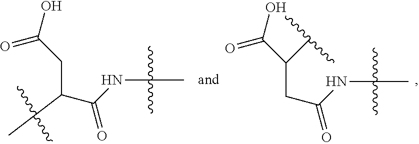 [SMILES: *NC(=O)C(*)CC(=O)O ; *NC(=O)CC(*)C(=O)O]

and likewise, depiction of the ring-open regioisomer

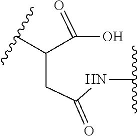 [SMILES: *NC(=O)CC(*)C(=O)O]

encompasses

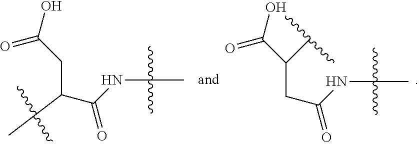 [SMILES: *NC(=O)C(*)CC(=O)O ; *NC(=O)CC(*)C(=O)O]

In some embodiments, a mixture of ring-open regioisomers is present. In some embodiments, a single ring-open regioisomer is present.

In some embodiments, each of s and t is 2.

In some embodiments, Spacer is a bond, **—NH—(CH₂CH₂O)ₘ—CH₂CH₂—C(═O)—, **—C(═O)—(CH₂CH₂O)ₘ—CH₂CH₂—NH—, or **—NH—(CH₂)ₘ—O—.

In some embodiments, Spacer is a bond, **—NH—(CH₂CH₂O)ₘ—CH₂CH₂—C(═O)—, or **—NH—(CH₂)ₘ—O—.

In some embodiments, m is 2, 4, 6, or 8.

In some embodiments, Conjugator has formula (IIa1), (IIa2), (IIa3), or (IIIa):

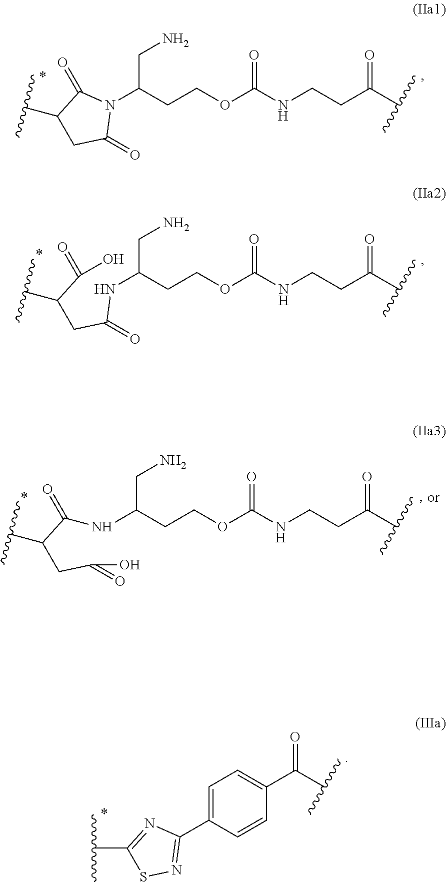 [SMILES: *C(=O)CCNC(=O)OCCC(CN)N1C(=O)CC(*)C1=O ; *C(=O)CCNC(=O)OCCC(CN)NC(=O)CC(*)C(=O)O ; *C(=O)CCNC(=O)OCCC(CN)NC(=O)C(*)CC(=O)O ; *C(=O)c1ccc(-c2nsc(*)n2)cc1]

In some embodiments, the Cleavable has one of the following formulas:

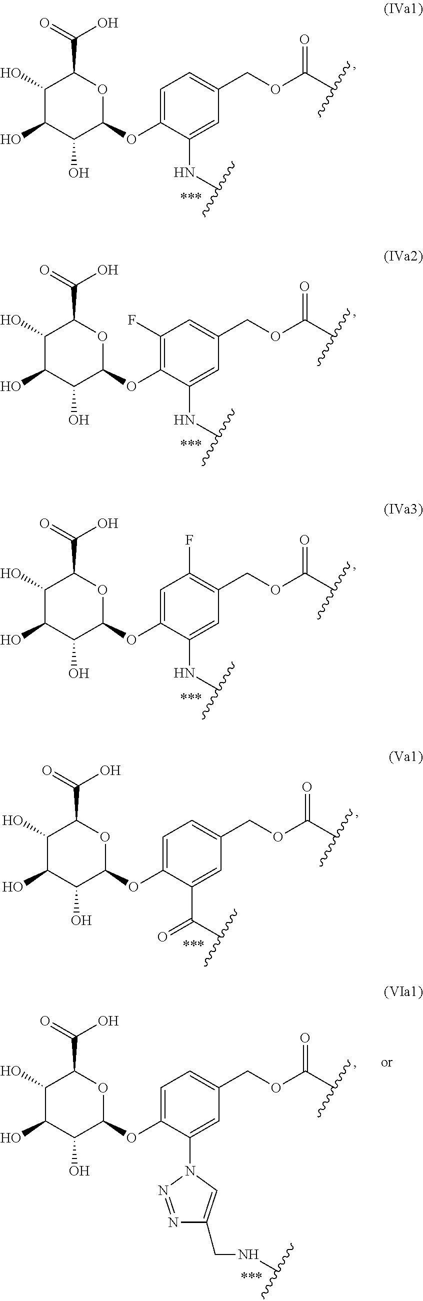 [SMILES: *Nc1cc(COC(*)=O)ccc1O[C@@H]1O[C@H](C(=O)O)[C@@H](O)[C@H](O)[C@H]1O ; *Nc1cc(COC(*)=O)cc(F)c1O[C@@H]1O[C@H](C(=O)O)[C@@H](O)[C@H](O)[C@H]1O ; *Nc1cc(COC(*)=O)c(F)cc1O[C@@H]1O[C@H](C(=O)O)[C@@H](O)[C@H](O)[C@H]1O ; *C(=O)OCc1ccc(O[C@@H]2O[C@H](C(=O)O)[C@@H](O)[C@H](O)[C@H]2O)c(C(*)=O)c1 ; *NCc1cn(-c2cc(COC(*)=O)ccc2O[C@@H]2O[C@H](C(=O)O)[C@@H](O)[C@H](O)[C@H]2O)nn1]

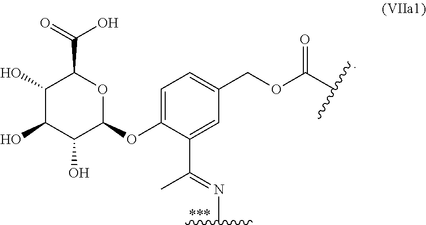 [SMILES: */N=C(\C)c1cc(COC(*)=O)ccc1O[C@@H]1O[C@H](C(=O)O)[C@@H](O)[C@H](O)[C@H]1O]

In some embodiments, Payload is a residue of one of the following formulas:

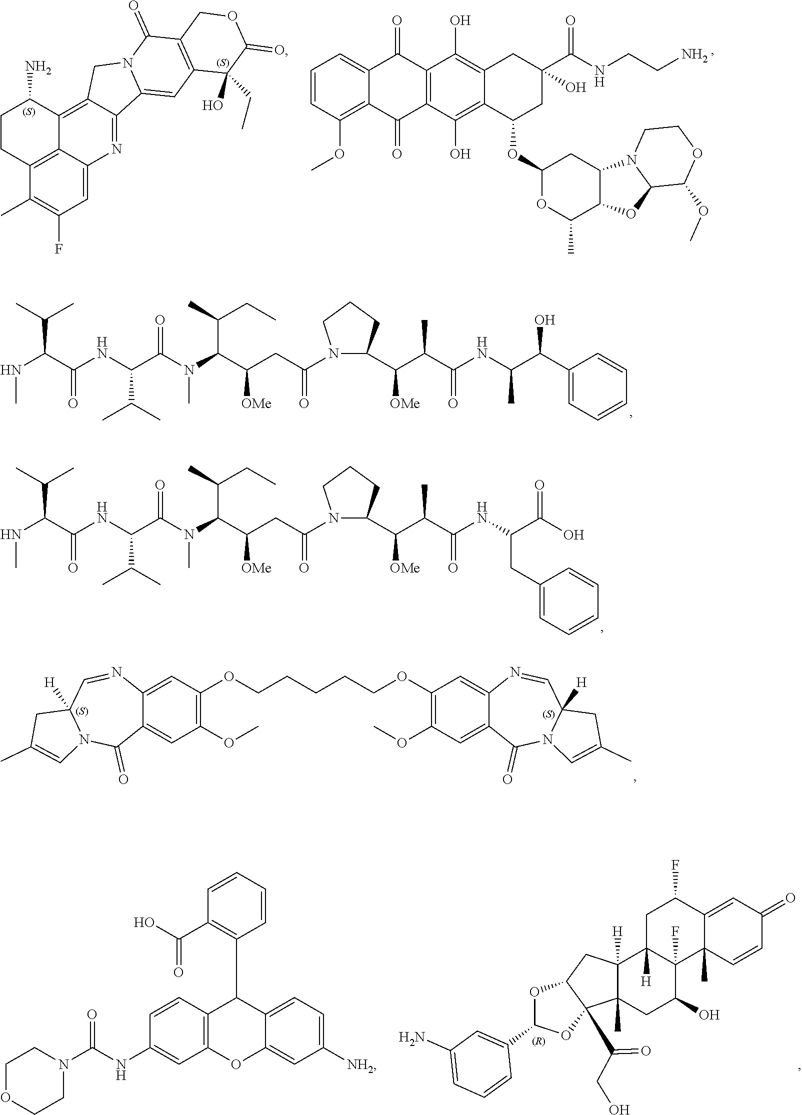

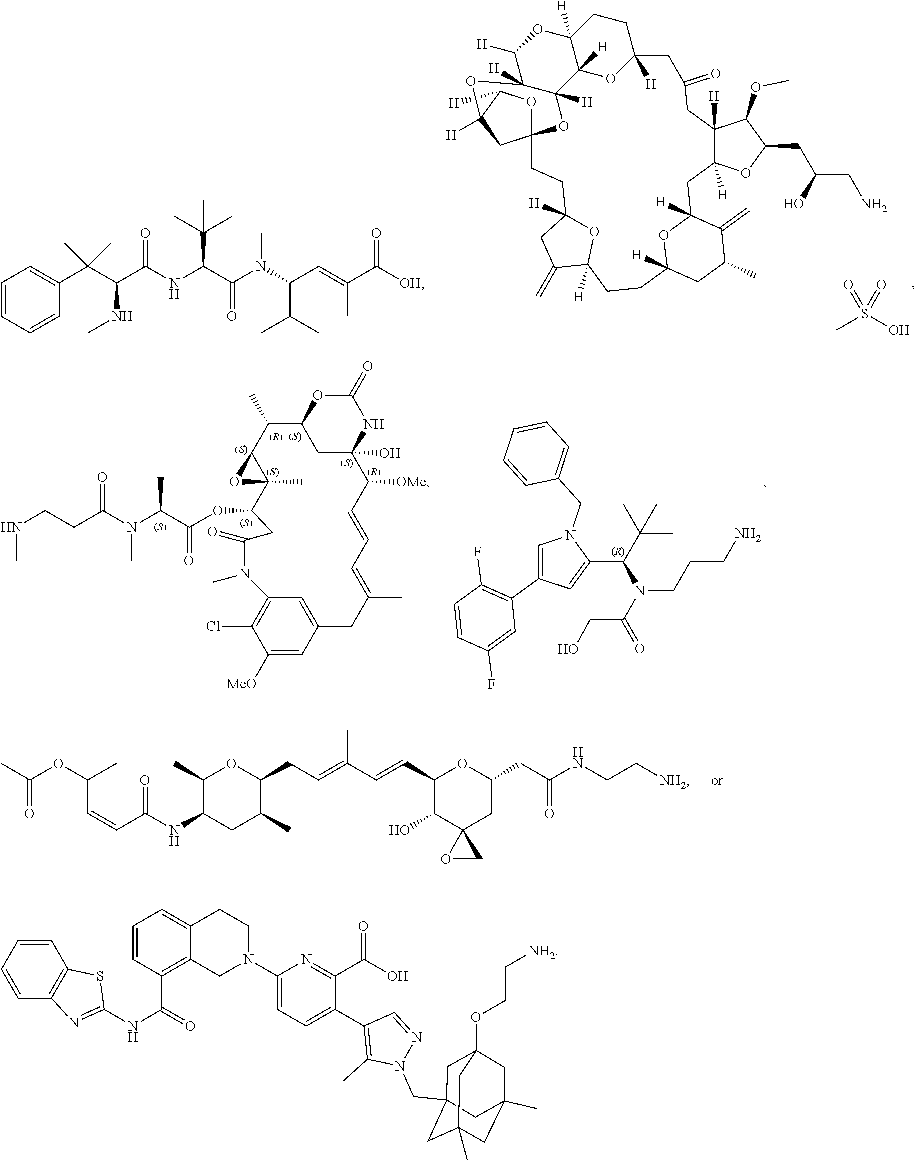

In some embodiments, Payload is

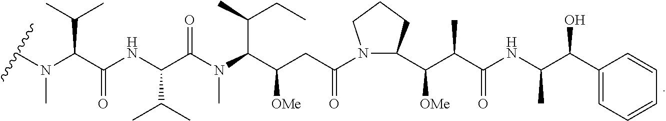 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

In some embodiments, the compound is

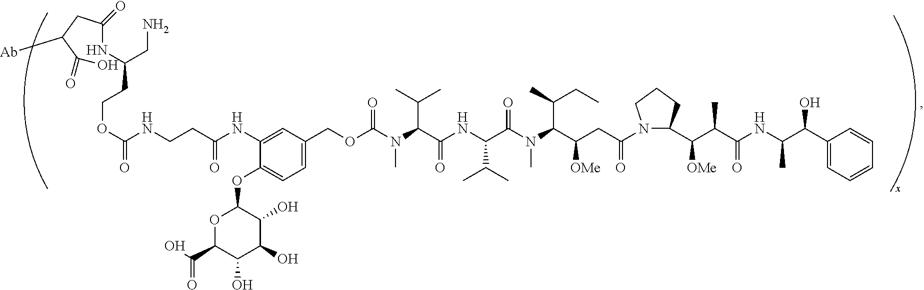

wherein Ab is 6E7 or an antigen-binding fragment thereof;

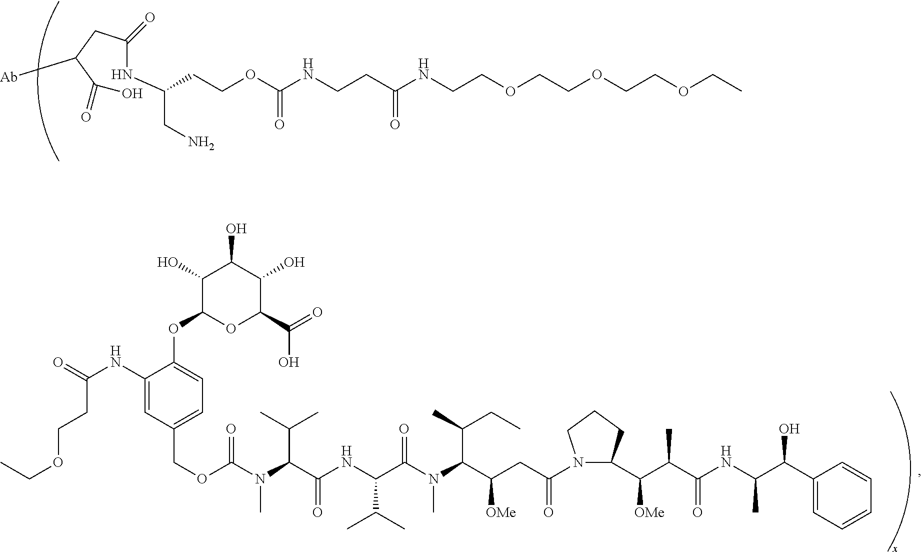

wherein Ab is 6E7 or an antigen-binding fragment thereof;

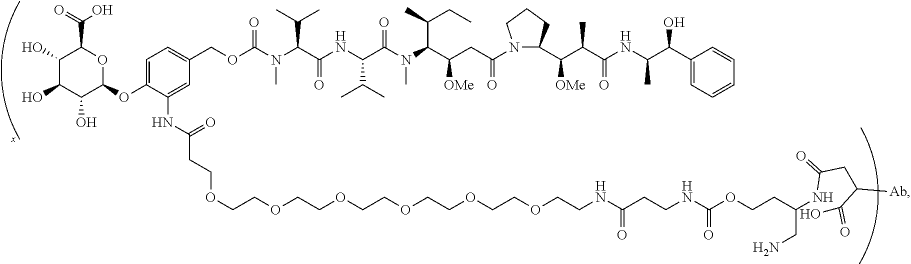

wherein Ab is 6E7 or an antigen-binding fragment thereof;

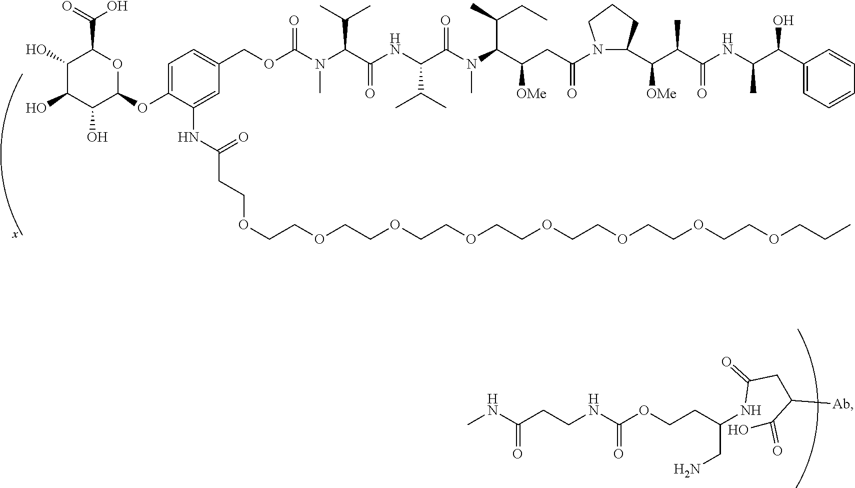

wherein Ab is 6E7 or an antigen-binding fragment thereof;

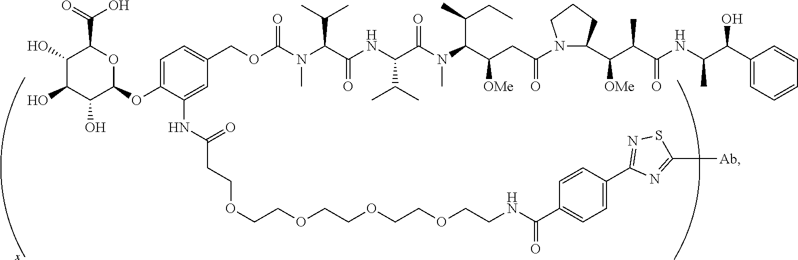

wherein Ab is 6E7 or an antigen-binding fragment thereof;

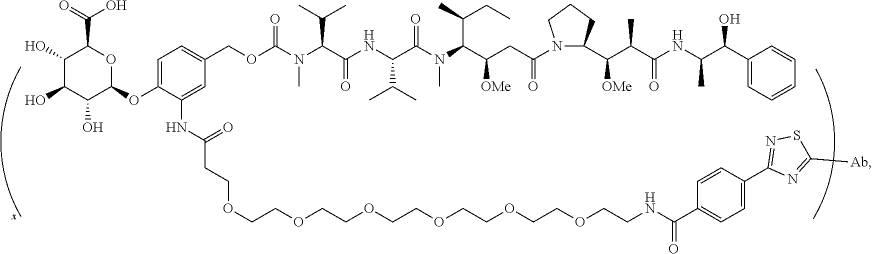

wherein Ab is 6E7 or an antigen-binding fragment thereof;

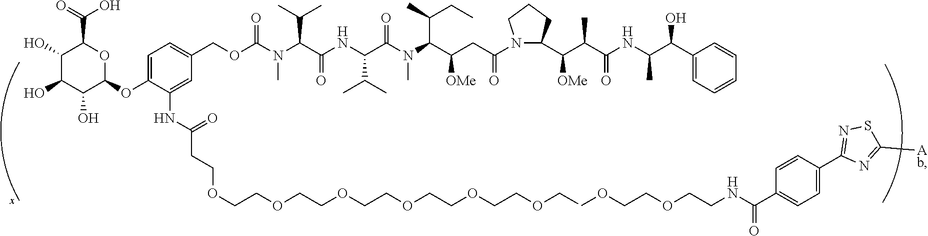

wherein Ab is 6E7 or an antigen-binding fragment thereof;

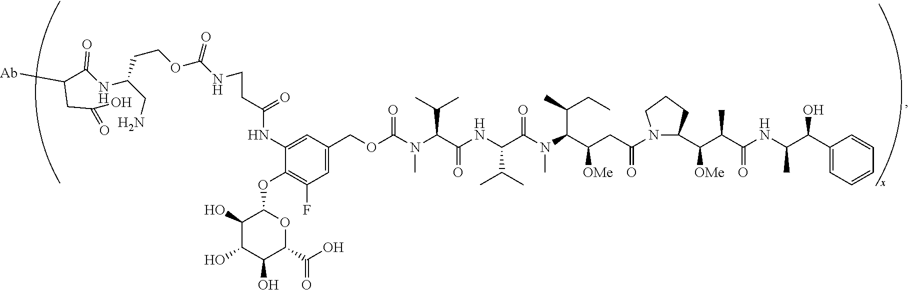

wherein Ab is 6E7 or an antigen-binding fragment thereof;

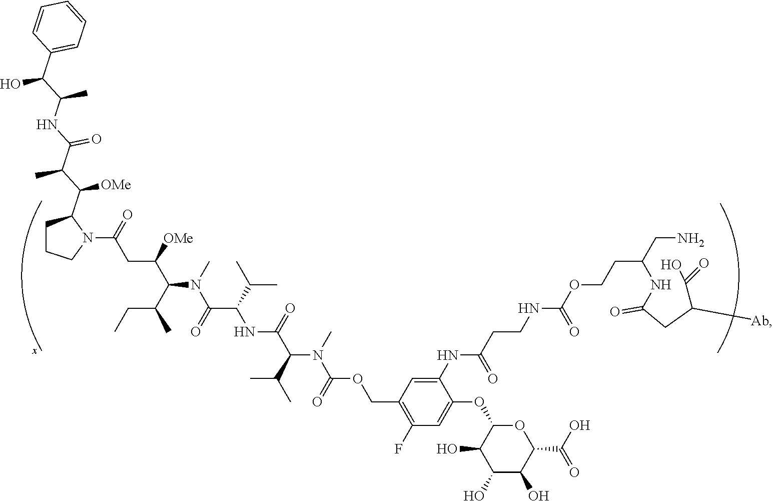

wherein Ab is 6E7 or an antigen-binding fragment thereof;

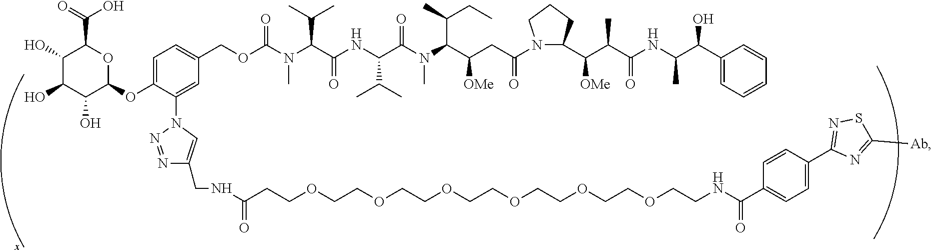

wherein Ab is 6E7 or an antigen-binding fragment thereof;

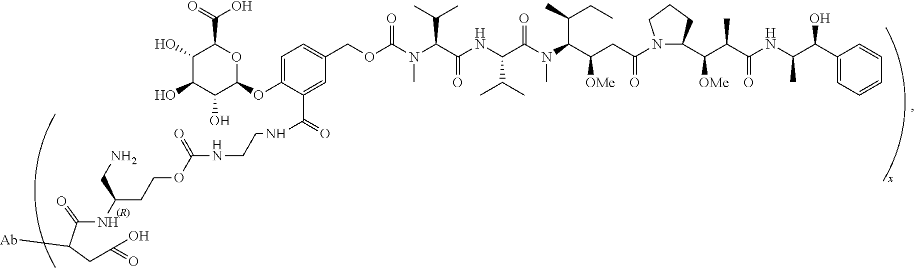

wherein Ab is 6E7 or an antigen-binding fragment thereof;

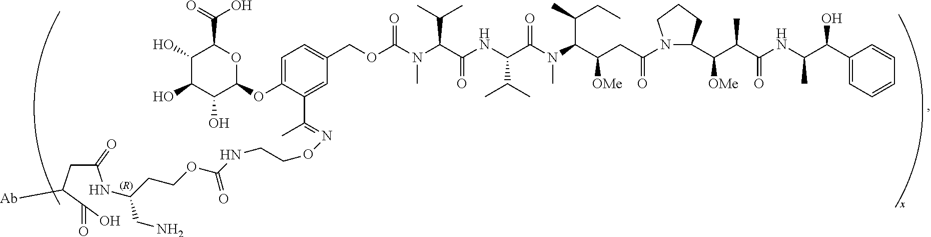

wherein Ab is 6E7 or an antigen-binding fragment thereof;

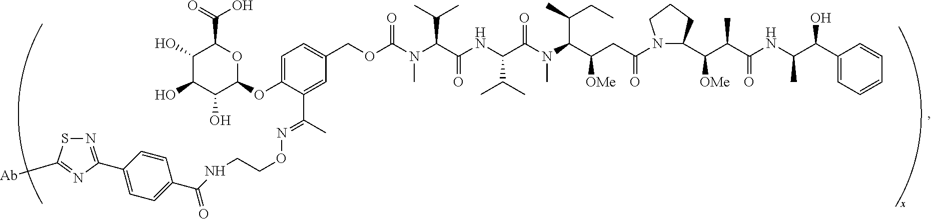

wherein Ab is 6E7 or an antigen-binding fragment thereof;

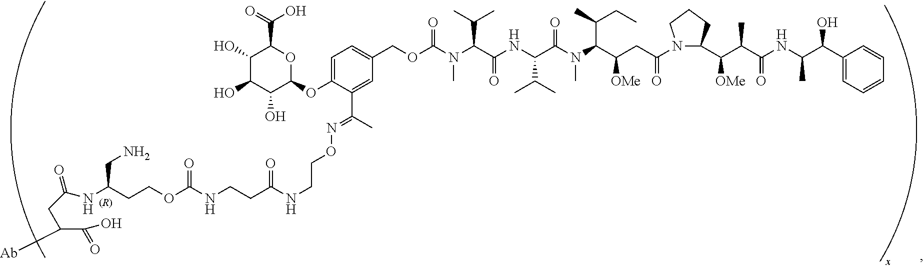

wherein Ab is 6E7 or an antigen-binding fragment thereof; or

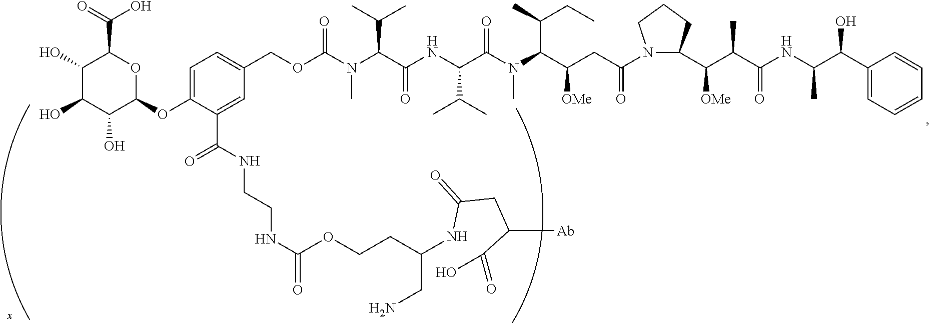

wherein Ab is ifinatamab or an antigen-binding fragment thereof,
or a pharmaceutically acceptable salt, tautomer, solvate, or stereoisomer of any of the foregoing.

In some embodiments, the compound is

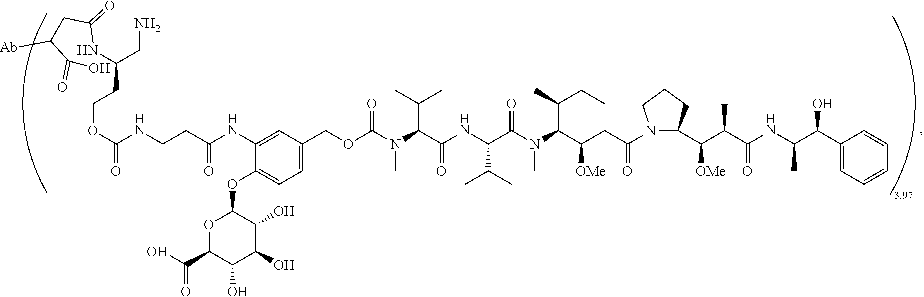

wherein Ab is 6E7 or an antigen-binding fragment thereof;

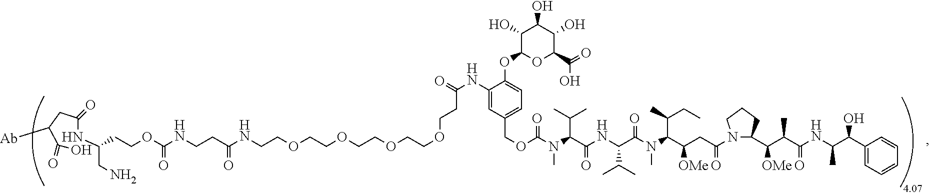

wherein Ab is 6E7 or an antigen-binding fragment thereof;

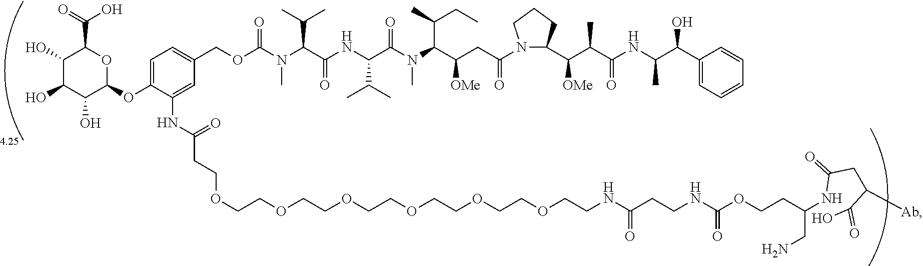

wherein Ab is 6E7 or an antigen-binding fragment thereof;

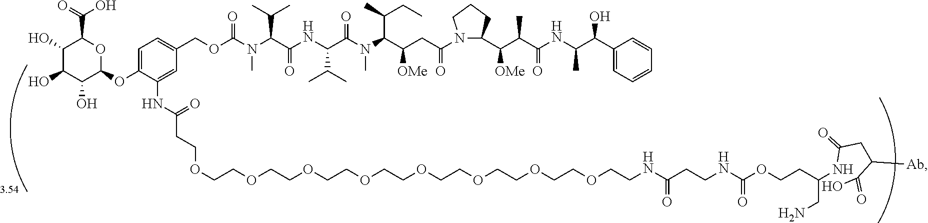

wherein Ab is 6E7 or an antigen-binding fragment thereof;

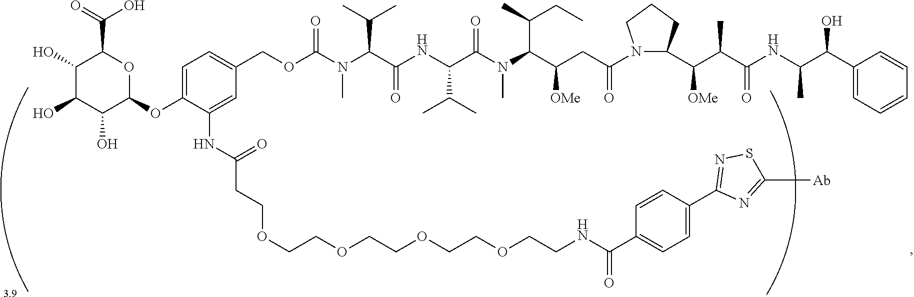

wherein Ab is 6E7 or an antigen-binding fragment thereof;

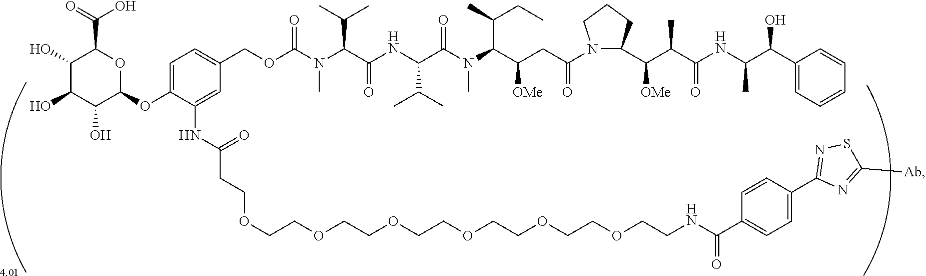

wherein Ab is 6E7 or an antigen-binding fragment thereof;

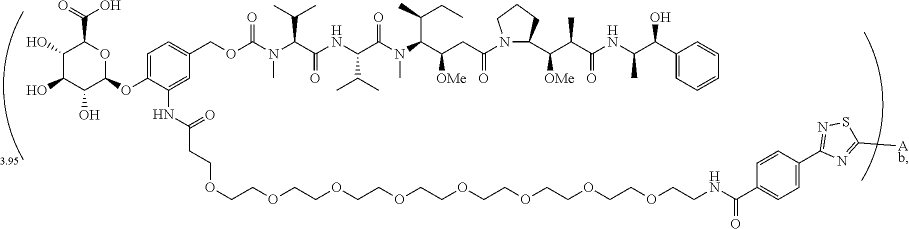

wherein Ab is 6E7 or an antigen-binding fragment thereof;

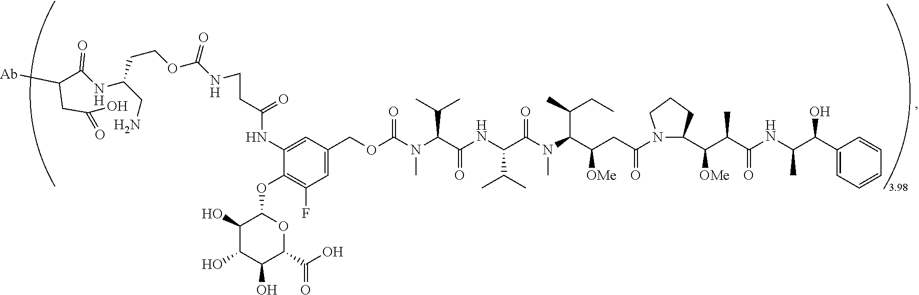

wherein Ab is 6E7 or an antigen-binding fragment thereof;

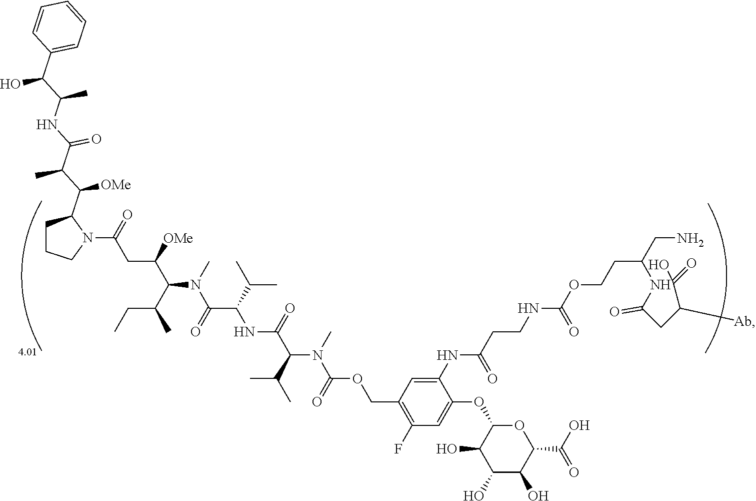

wherein Ab is 6E7 or an antigen-binding fragment thereof;

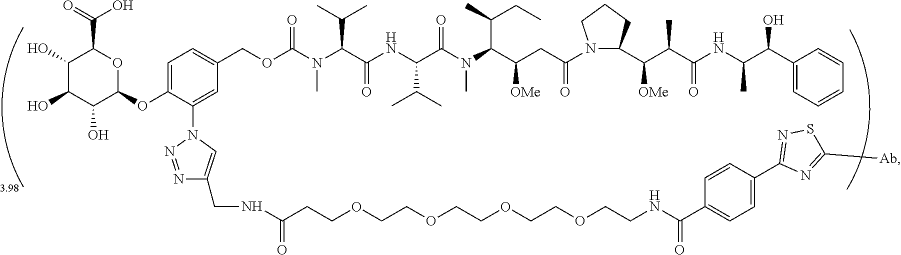

wherein Ab is 6E7 or an antigen-binding fragment thereof;

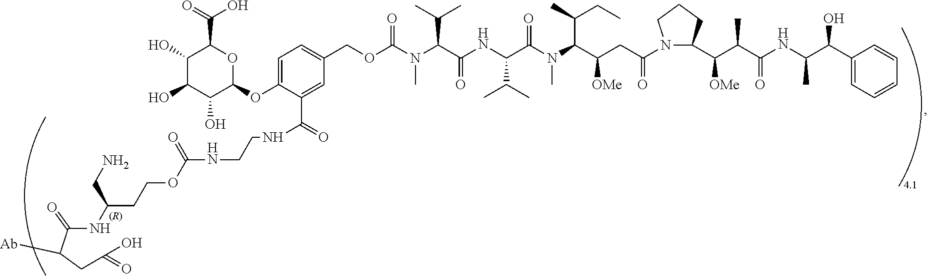

wherein Ab is 6E7 or an antigen-binding fragment thereof;

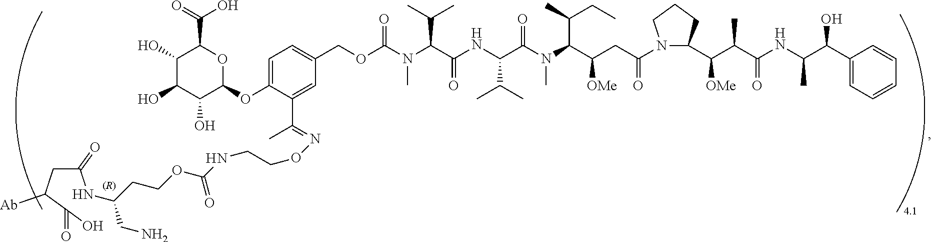

wherein Ab is 6E7 or an antigen-binding fragment thereof;

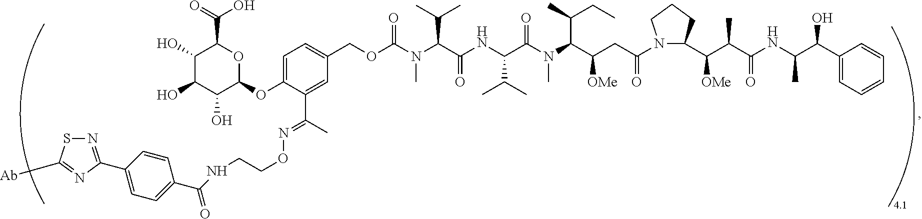

wherein Ab is 6E7 or an antigen-binding fragment thereof;

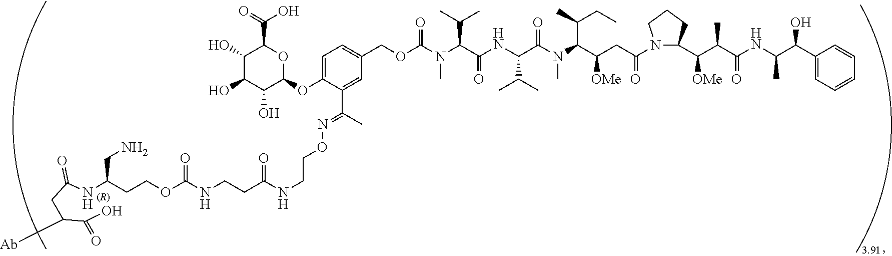

wherein Ab is 6E7 or an antigen-binding fragment thereof; or

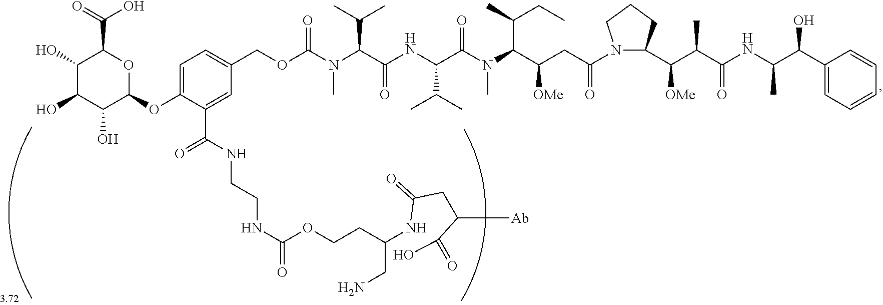

wherein Ab is ifinatamab or an antigen-binding fragment thereof,
or a pharmaceutically acceptable salt, tautomer, solvate, or stereoisomer of any of the foregoing.

In one embodiment, x is from 1 to 15 inclusive. In one embodiment, x is from 1 to 12. In one embodiment, x is from 1 to 10. In one embodiment, x is from 2 to 10. In one embodiment, x is from 3.5 to 10. In one embodiment, x is from 3.5 to 8. In one embodiment, x is from 3.5 to 6. In one embodiment, x is from 3.5 to 4.5.

### Binding Agents

Provided herein are binding agents (BA or Ab), e.g., for use in an ADC described herein.

Compounds of formula (I) may include any BA described herein.

In some embodiments, BA is an antibody or antigen-binding fragment thereof, e.g., a humanized, chimeric, or human antibody or an antigen-binding fragment thereof.

In some embodiments, the antibody or antigen-binding fragment thereof is a monoclonal antibody, a chimeric antibody, a humanized antibody, a human engineered antibody, a single chain antibody (scFv), a Fab fragment, a Fab′ fragment, or a F(ab′)2 fragment.

In some embodiments, BA is an antibody or an antigen-binding fragment thereof that binds to one or more of B7H3, cytokeratin 15, PTK7, HER3, HER2, CD7, CD19, CD20, CD22, CD25, CD27, CD30, CD33, CD37, CD38, CD46, CD70, CD71, CD74, CD79b, CD123, CD138, CD142, CD166, CD205, CD228, CCR2, CA6, p-Cadherin, CEA, CEACAM5, C4.4a, DLL3, EGFR, EGFRVIII, ENPP3, EphA2, EphrinA, FLOR1, FGFR2, GCC, cKIT, LIV1, LY6E, MSLN, MUC16, NaPi2b, Nectin4, gpNMB, PSMA, SLITRK6, STEAP1, TROP2, 5T4, SSEA4, GloboH, Gb5, STn, Tn, B7H3, BCMA, MUC1, cMet, ROR1 MSLn, FRa, CLDN18.2, CLDN6, PTK7, Axl, FGFR2b, CLL1, CCR7, GPC1, GPC3, ISAC, CDCP1, ITGB6, ADAM9, or CD45-iADC.

In some embodiments, BA is an anti-human B7H3 antibody, an anti-CLL1 antibody, an anti-PTK7 antibody, an anti-HER3 antibody, an anti-HER2 antibody, or an antigen-binding fragment of any of the foregoing. In some embodiments, the anti-human B7H3 antibody is ifinatamab. In some embodiments, the anti-CLL1 antibody is 6E7. In some embodiments, the anti-PTK7 antibody is cofetuzumab. In some embodiments, the anti-HER3 antibody is patritumab. In some embodiments, the anti-HER2 antibody is trastuzumab.

In some embodiments, the antibody or antigen-binding fragment thereof specifically binds human B7H3. In some embodiments, the antibody or antigen-binding fragment thereof is ifinatamab.

In some embodiments, the antibody or antigen-binding fragment thereof specifically binds CLL1. In some embodiments, the antibody or antigen-binding fragment thereof is 6E7.

In some embodiments, the antibody or antigen-binding fragment thereof specifically binds PTK7. In some embodiments, the antibody or antigen-binding fragment thereof is cofetuzumab.

### Aspect 2

Provided herein are compounds of formula (XI):

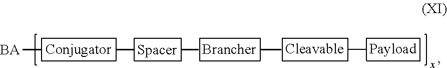

or a pharmaceutically acceptable salt, tautomer, solvate, or stereoisomer thereof, wherein:
BA is a binding agent selected from a humanized, monoclonal, chimeric, or human antibody or an antigen-binding fragment thereof.
Conjugator has formula (II) or (III):

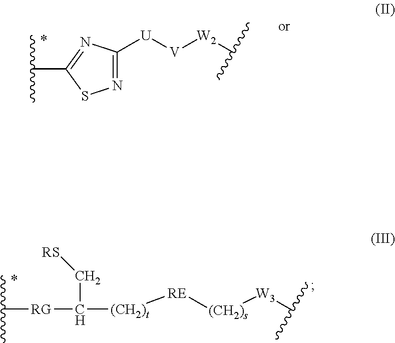

U is a bond, heteroarylene, or arylene;
V is a bond or —C≡C—(CH₂)ₙ—;
n is an integer from 0 to 10 inclusive;
W₂ is —C(═O)—, —NH—, or —O—;
RG is

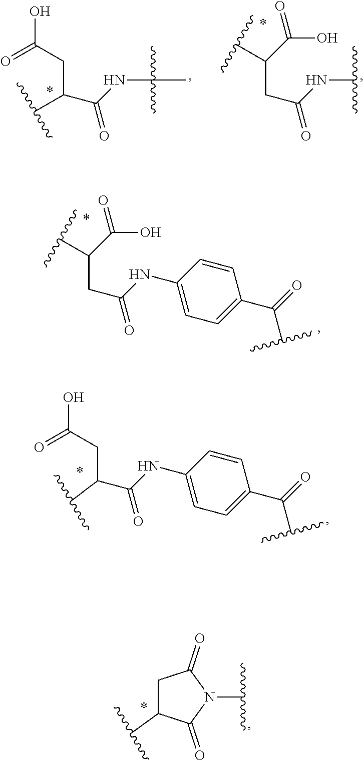 [SMILES: *NC(=O)C(*)CC(=O)O ; *NC(=O)CC(*)C(=O)O ; *C(=O)c1ccc(NC(=O)CC(*)C(=O)O)cc1 ; *C(=O)c1ccc(NC(=O)C(*)CC(=O)O)cc1 ; *C1CC(=O)N(*)C1=O]

succinimid-3-yl-N)—, or

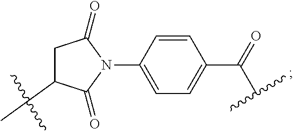 [SMILES: *C(=O)c1ccc(N2C(=O)CC(*)C2=O)cc1]

RS is —NR^{1a}R^{1b};
each of R^{1a} and R^{1b} is, independently, H or substituted or unsubstituted C₁₋₄ alkyl;
RE is a bond, —O—, —OC(═O)—, —OC(═O)NR⁶—, —NHC(═O)NR⁶—, —OS(═O)₂NR⁶—, —NHS(═O)₂NR⁶—, —or —OC(═O)NHS(═O)₂NR⁶—;
R⁶ is H or substituted or unsubstituted C₁₋₄ alkyl;
W₃ is —C(═O)—, —NH—, or —O—;
each of s and t is, independently, 0, 1, or 2;
[symbol] indicates a point of covalent attachment within the compound;
* marks the bond where Conjugator connects to BA;
Spacer is a bond, **—NH—(CH₂CH₂O)ₘ—CH₂CH₂—C(═O)—, **—C(═O)—(CH₂CH₂O)ₘ—CH₂CH₂—NH—, **—(CH₂)ₘ—C(═O)—, **—CH₂—C(═O)—NH—(CH₂)ₘ—C(═O)—, **—(CH₂CH₂O)ₘ—CH₂CH₂—C(═O)—, **—C(═O)—(CH₂CH₂O)ₘ—CH₂CH₂—NH—, **—CH[—(CH₂)ₘ—COOH]—C(═O)—, **—CH₂—C(═O)—NH—(CH₂)ₘ—C(═O)—NH—(CH₂)ₘ—C(═O)—, **—C(═O)—(CH₂)ₘ—C(═O)—, **—NH—(CH₂)ₘ—C(═O)—, or **—NH—(CH₂)ₘ—O—;
m is an integer from 1 and 12 inclusive;
** marks the bond where Spacer connects to Conjugator;
Cleavable has formula (IVa′), (IVb′), (IVc′), (Va′), (Vb′), (Via′), (VIb′), (VIIa′), or (VIIb′):

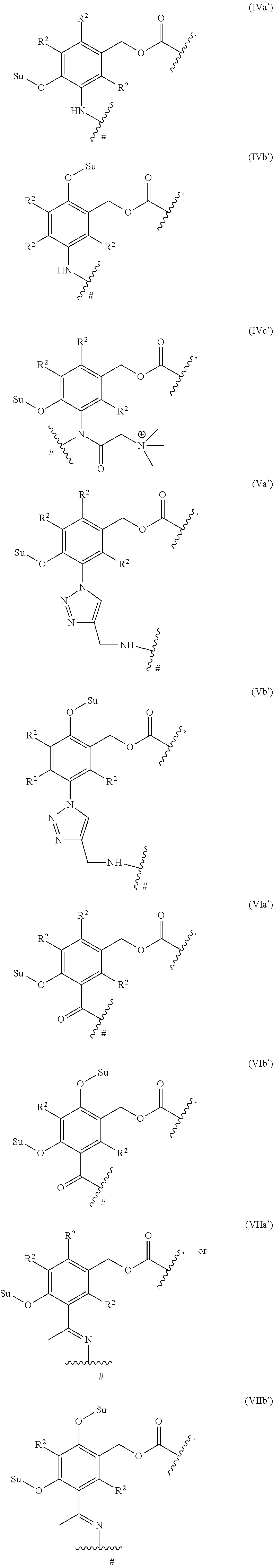

Su is a Sugar moiety;
each R² is, independently, hydrogen, halogen, substituted or unsubstituted C₁₋₄ alkyl, —CN, or —NO₂;
#marks the bond where Cleavable connects to Brancher,
Brancher is selected from formula (XIIa), (XIIb), (XIIc), and (XIId):

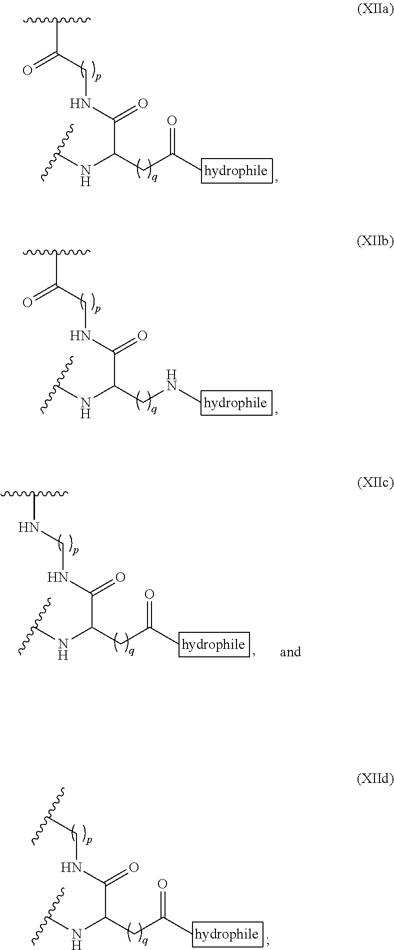

each of p and q is, independently, 1, 2, 3, or 4;
hydrophile is —NH—(CH₂CH₂O)ₐ—(CH₂)_{b}CH₃, —N(CH₃)—((CH₂)_{c}C(═O) N(CH₃))ₐ—(CH₂)_{c}C(═O) NH₂, —C(═O)—(CH₂CH₂O)ₐ—(CH₂)_{b}CH₃, —C(═O)—(CH₂CH₂O)ₐ—(CH₂)_{b}NHC(═O) (CH₂)₃N(CH₃)₂ (CH₂)₃S(═O)₂OH, —NH—(CH₂CH₂O)ₐ—(CH₂)_{b}NHC(═O) (CH₂)₃N(CH₃)₂ (CH₂)₃S(═O)₂OH,

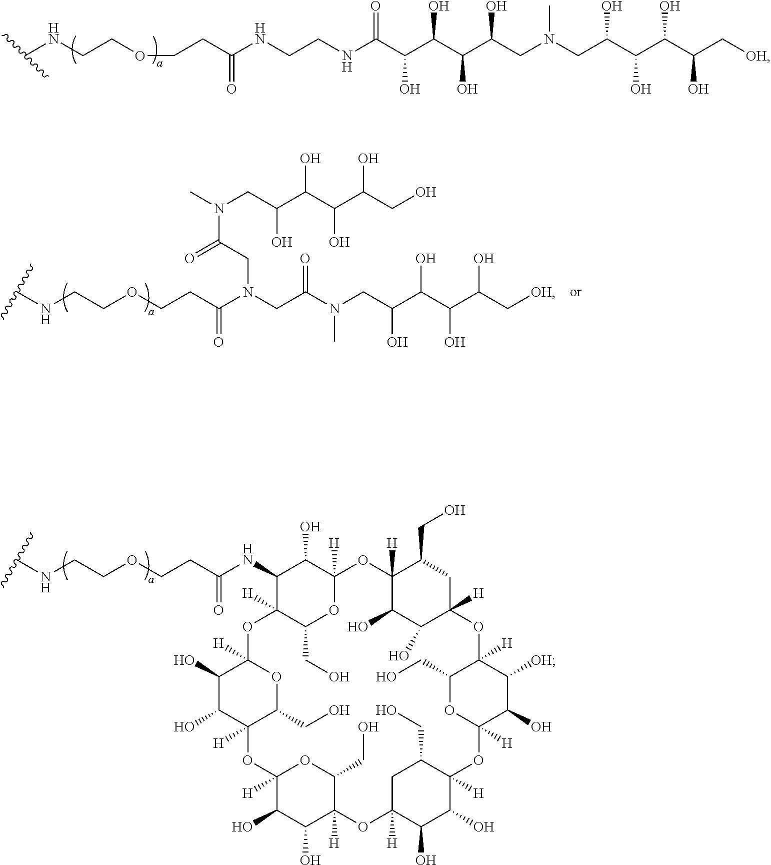

a is an integer from 1 to 18 inclusive;
b is 0, 1, or 2;
c is 1, 2, 3, or 4;
Payload is a payload residue; and
x is from 1 to 15 inclusive.

In some embodiments, Conjugator has formula (II).

In some embodiments, U is arylene.

In some embodiments, U is phenylene.

In some embodiments, U is

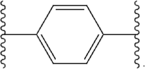 [SMILES: *c1ccc(*)cc1]

In some embodiments, U is arylene and V is a bond.

In some embodiments, U is arylene and V is —C≡C—(CH₂)ₙ—.

In some embodiments, U is arylene and V is —C≡C—(CH₂)₃—.

In some embodiments, U is heteroarylene.

In some embodiments, U is a bivalent pyrimidine ring.

In some embodiments, U is

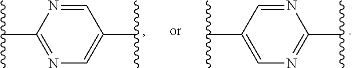 [SMILES: *c1cnc(*)nc1 ; *c1cnc(*)nc1]

In some embodiments, U is heteroarylene and V is a bond.

In some embodiments, U is heteroarylene and V is —C≡C—(CH₂)ₙ—.

In some embodiments, U is heteroarylene and V is —C≡C—(CH₂)₃—.

In some embodiments, U is a bond.

In some embodiments, U is a bond and V is a bond.

In some embodiments, U is a bond and V is —C≡C—(CH₂)ₙ—.

In some embodiments, U is a bond and V is —C≡C—(CH₂)₃—.

In some embodiments, W₂ is —C(═O)—.

In some embodiments, Conjugator has formula (III).

In some embodiments, RS is —NH₂ or —N(CH₃)₂.

In some embodiments, RS is —NH₂.

In some embodiments, RE is —OC(═O) NH—.

In some embodiments, RG is

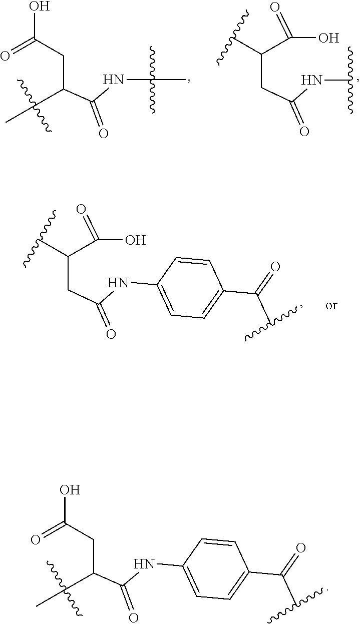 [SMILES: *NC(=O)C(*)CC(=O)O ; *NC(=O)CC(*)C(=O)O ; *C(=O)c1ccc(NC(=O)CC(*)C(=O)O)cc1 ; *C(=O)c1ccc(NC(=O)C(*)CC(=O)O)cc1]

In some embodiments, RG is

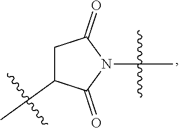 [SMILES: *C1CC(=O)N(*)C1=O]

-(succinimid-3-yl-N)—, or

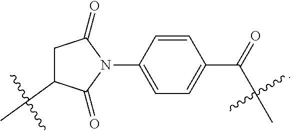 [SMILES: *C(=O)c1ccc(N2C(=O)CC(*)C2=O)cc1]

In some embodiments, RG is

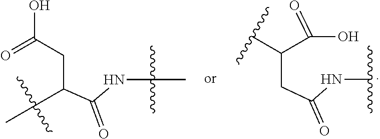 [SMILES: *NC(=O)C(*)CC(=O)O ; *NC(=O)CC(*)C(=O)O]

Certain RG described herein, e.g.,

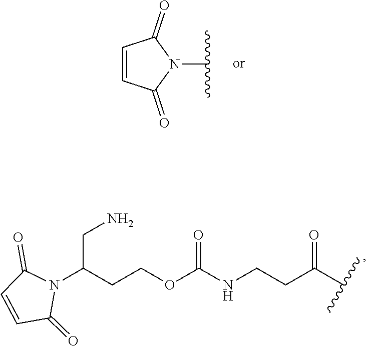 [SMILES: *N1C(=O)C=CC1=O ; *C(=O)CCNC(=O)OCCC(CN)N1C(=O)C=CC1=O]

may, upon conjugation with BA, be induced to undergo a ring opening process. During such process the

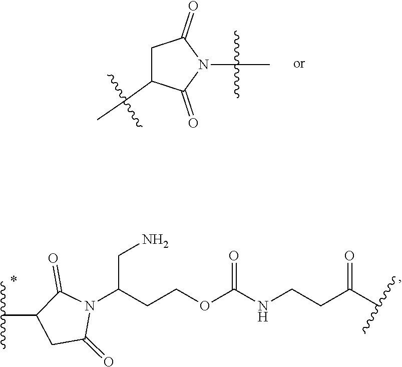 [SMILES: *C1CC(=O)N(*)C1=O ; *C(=O)CCNC(=O)OCCC(CN)N1C(=O)CC(*)C1=O]

maleimide structure formed upon conjugation of the RG with BA, e.g.,

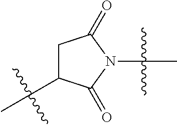 [SMILES: *C1CC(=O)N(*)C1=O]

undergoes ring opening. It will be appreciated that there are two possible regioisomers that may result from the ring opening of such maleimide structures. For example,

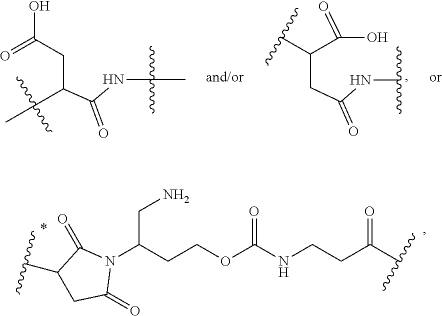 [SMILES: *NC(=O)C(*)CC(=O)O ; *NC(=O)CC(*)C(=O)O ; *C(=O)CCNC(=O)OCCC(CN)N1C(=O)CC(*)C1=O]

may undergo ring opening to form

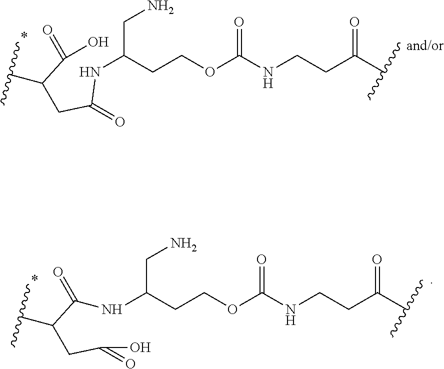 [SMILES: *C(=O)CCNC(=O)OCCC(CN)NC(=O)CC(*)C(=O)O ; *C(=O)CCNC(=O)OCCC(CN)NC(=O)C(*)CC(=O)O]

For simplicity, unless indicated otherwise, both possible ring-open regioisomers are encompassed by a depiction of a single ring-open regioisomer herein. Thus, unless indicated otherwise, depiction of the ring-open regioisomer

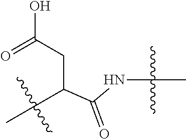 [SMILES: *NC(=O)C(*)CC(=O)O]

encompasses

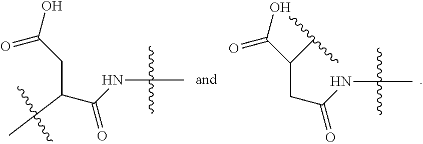 [SMILES: *NC(=O)C(*)CC(=O)O ; *NC(=O)CC(*)C(=O)O]

and likewise, depiction of the ring-open regioisomer

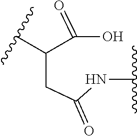 [SMILES: *NC(=O)CC(*)C(=O)O]

encompasses

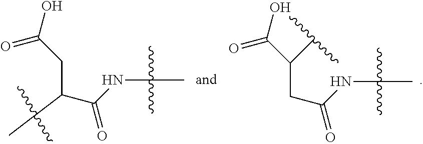 [SMILES: *NC(=O)C(*)CC(=O)O ; *NC(=O)CC(*)C(=O)O]

In some embodiments, a mixture of ring-open regioisomers is present. In some embodiments, a single ring-open regioisomer is present.

In some embodiments, each of s and t is, independently, 0 or 2.

In some embodiments, W₃ is —C(═O)—.

In some embodiments, Spacer is a bond, **—NH—(CH₂CH₂O)ₘ—CH₂CH₂—C(═O)—, or **—C(═O)—(CH₂CH₂O)ₘ—CH₂CH₂—NH—. In some embodiments, Spacer is a bond.

In some embodiments, m is 2, 4, 6, or 8.

In some embodiments, Conjugator has formula (IIa1), (IIa2), (IIa3), (IIa4), (IIa5), or (IIIa):

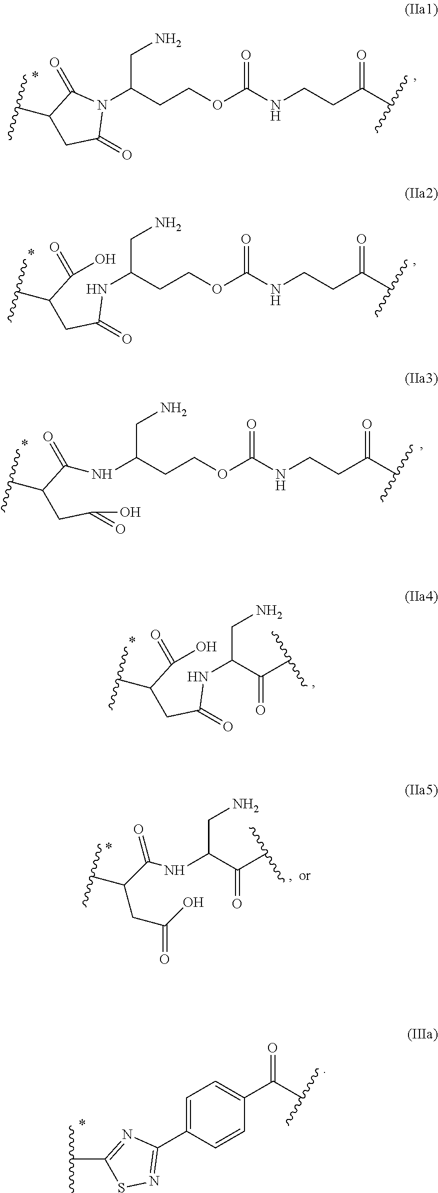 [SMILES: *C(=O)CCNC(=O)OCCC(CN)N1C(=O)CC(*)C1=O ; *C(=O)CCNC(=O)OCCC(CN)NC(=O)CC(*)C(=O)O ; *C(=O)CCNC(=O)OCCC(CN)NC(=O)C(*)CC(=O)O ; *C(=O)C(CN)NC(=O)CC(*)C(=O)O ; *C(=O)C(CN)NC(=O)C(*)CC(=O)O ; *C(=O)c1ccc(-c2nsc(*)n2)cc1]

In some embodiments, Cleavable has one of the following formulas:

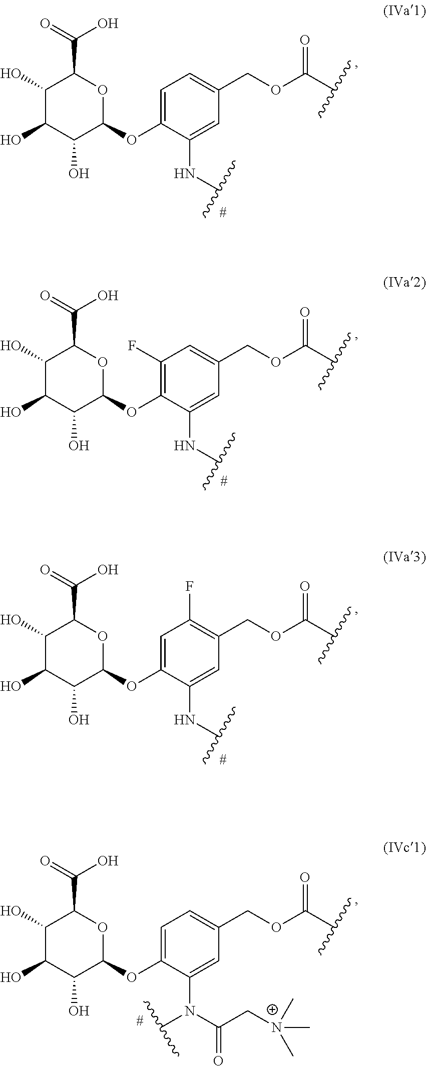 [SMILES: *Nc1cc(COC(*)=O)ccc1O[C@@H]1O[C@H](C(=O)O)[C@@H](O)[C@H](O)[C@H]1O ; *Nc1cc(COC(*)=O)cc(F)c1O[C@@H]1O[C@H](C(=O)O)[C@@H](O)[C@H](O)[C@H]1O ; *Nc1cc(COC(*)=O)c(F)cc1O[C@@H]1O[C@H](C(=O)O)[C@@H](O)[C@H](O)[C@H]1O ; *C(=O)OCc1ccc(O[C@@H]2O[C@H](C(=O)O)[C@@H](O)[C@H](O)[C@H]2O)c(N(*)C(=O)C[N+](C)(C)C)c1]

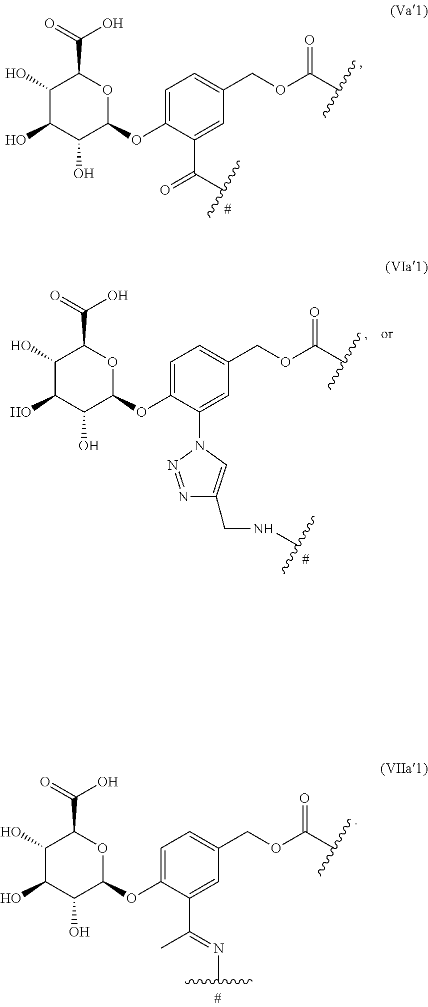 [SMILES: *C(=O)OCc1ccc(O[C@@H]2O[C@H](C(=O)O)[C@@H](O)[C@H](O)[C@H]2O)c(C(*)=O)c1 ; *NCc1cn(-c2cc(COC(*)=O)ccc2O[C@@H]2O[C@H](C(=O)O)[C@@H](O)[C@H](O)[C@H]2O)nn1 ; */N=C(\C)c1cc(COC(*)=O)ccc1O[C@@H]1O[C@H](C(=O)O)[C@@H](O)[C@H](O)[C@H]1O]

In some embodiments, Cleavable has formula (IVa′1), (IVc′1), or (Va′1):

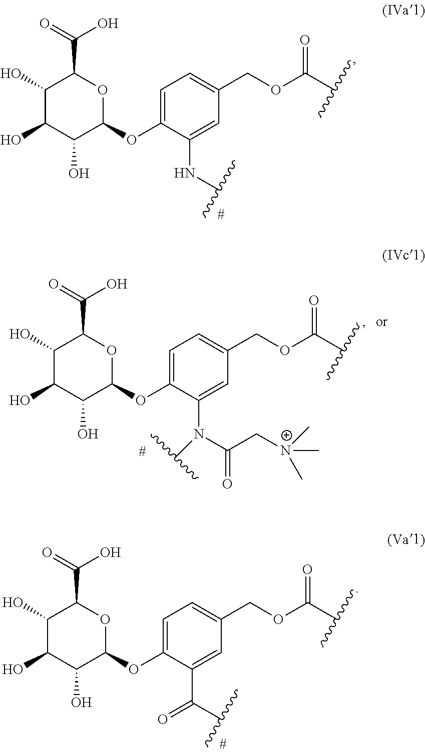 [SMILES: *Nc1cc(COC(*)=O)ccc1O[C@@H]1O[C@H](C(=O)O)[C@@H](O)[C@H](O)[C@H]1O ; *C(=O)OCc1ccc(O[C@@H]2O[C@H](C(=O)O)[C@@H](O)[C@H](O)[C@H]2O)c(N(*)C(=O)C[N+](C)(C)C)c1 ; *C(=O)OCc1ccc(O[C@@H]2O[C@H](C(=O)O)[C@@H](O)[C@H](O)[C@H]2O)c(C(*)=O)c1]

In some embodiments, hydrophile is —NH—(CH₂CH₂O)ₐ—(CH₂)_{b}CH₃, —N(CH₃)—((CH₂)_{c}C(═O) N(CH₃))ₐ—(CH₂)_{c}C(═O) NH₂, —C(═O)—(CH₂CH₂O)ₐ—(CH₂)_{b}CH₃, —C(═O)—(CH₂CH₂O)ₐ—(CH₂)_{b}NHC(═O) (CH₂)₃N(CH₃)₂ (CH₂)₃S(═O)₂OH,

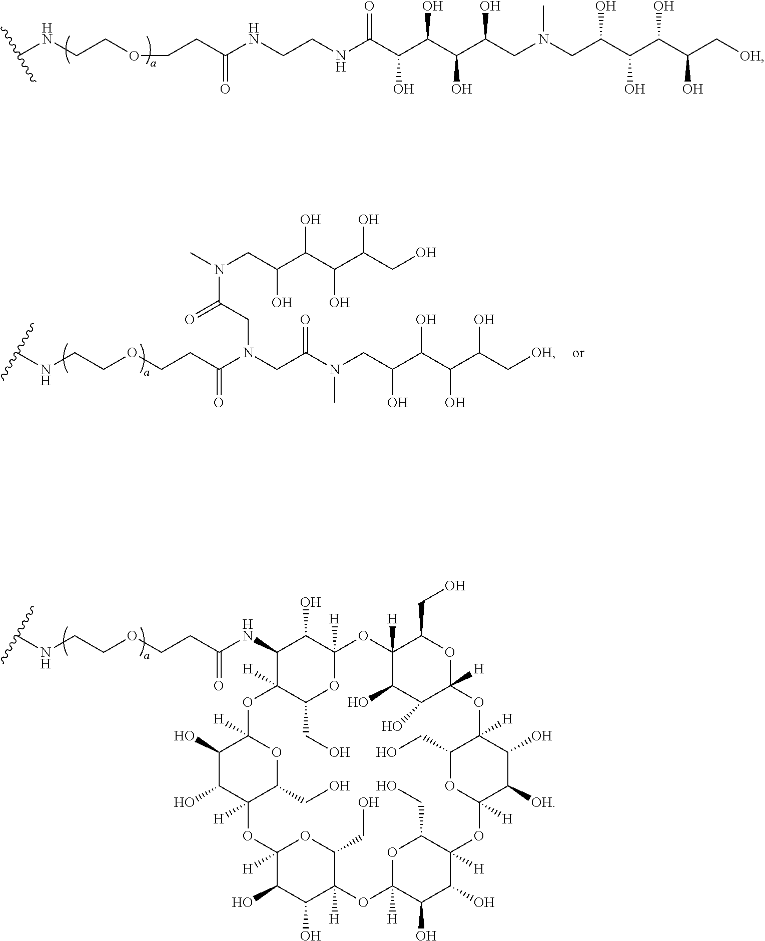

In some embodiments, a is 4, 8, 11, or 12.

In some embodiments, b is 0 or 2.

In some embodiments, c is 1.

In some embodiments, a is 12 and b is 0.

In some embodiments, hydrophile is —NH—(CH₂CH₂O)₁₂—CH₃, —N(CH₃)—((CH₂)C(═O) N(CH₃))₁₁—(CH₂)_{c}C(═O) NH₂, —C(═O)—(CH₂CH₂O)₁₂—CH₃, —C(═O)—(CH₂CH₂O)₈—(CH₂)₂NHC(═O) (CH₂)₃N(CH₃)₂ (CH₂)₃S(═O)₂OH,

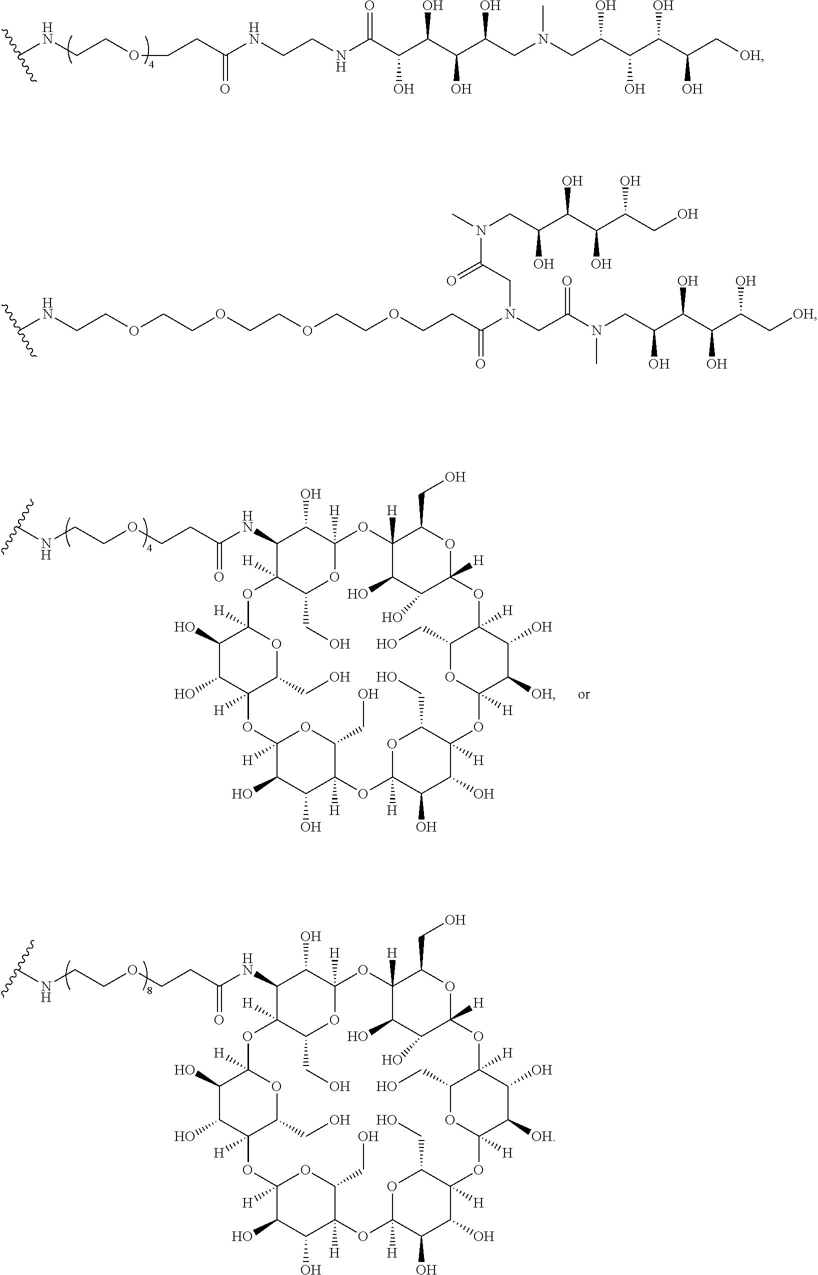

In some embodiments, each of p and q is, independently, 2 or 4.

In some embodiments, Payload is a residue of:

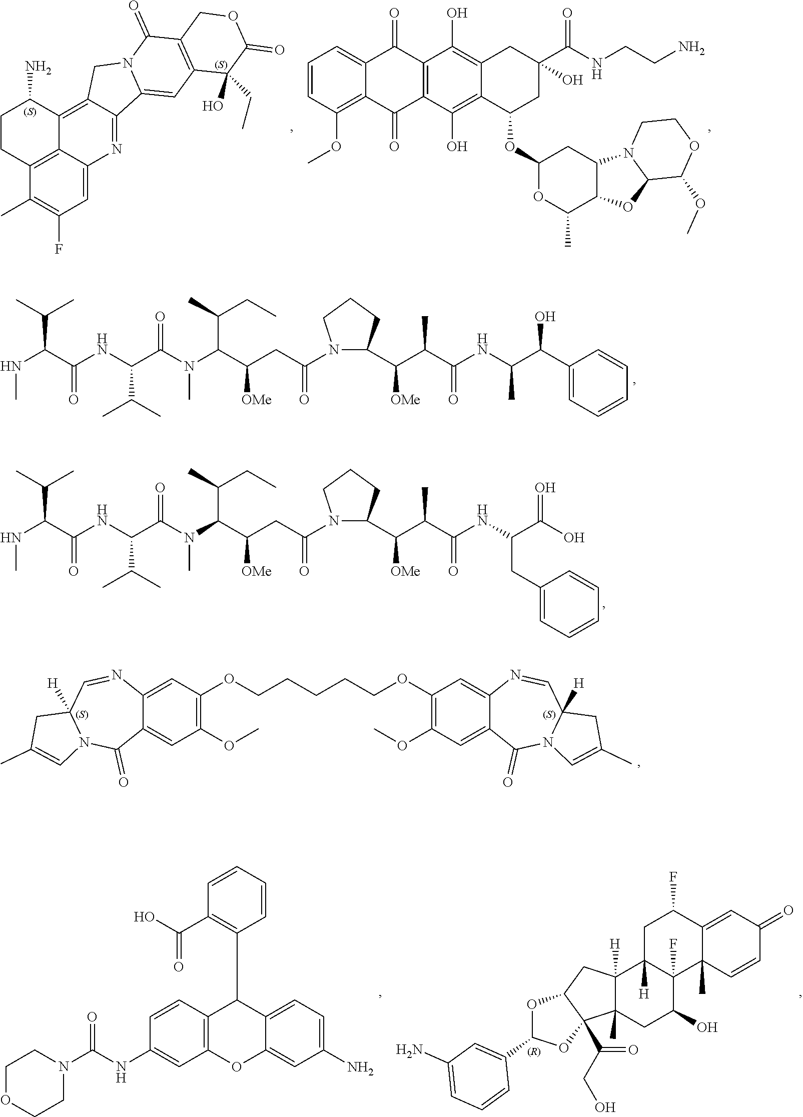

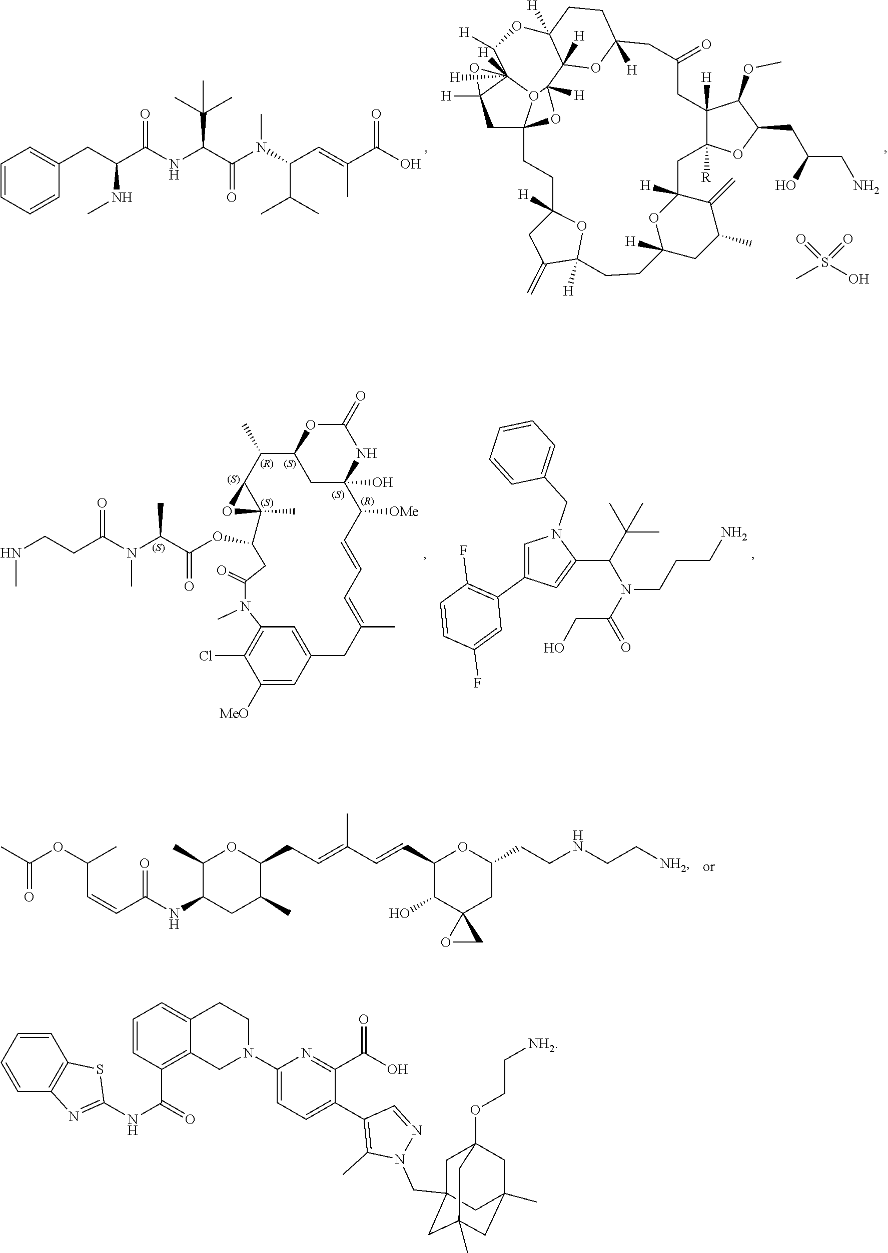

In some embodiments, Payload is

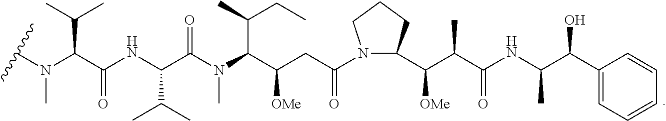 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

In some embodiments, the compound is

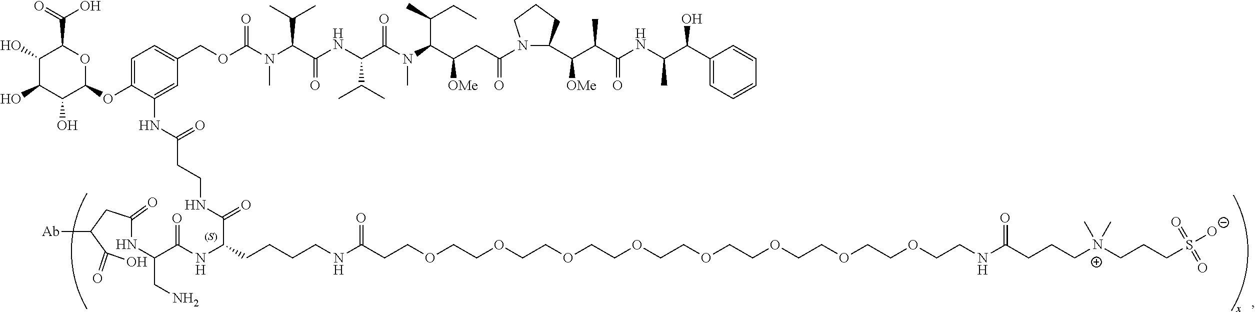

wherein Ab is 6E7 or an antigen-binding fragment thereof;

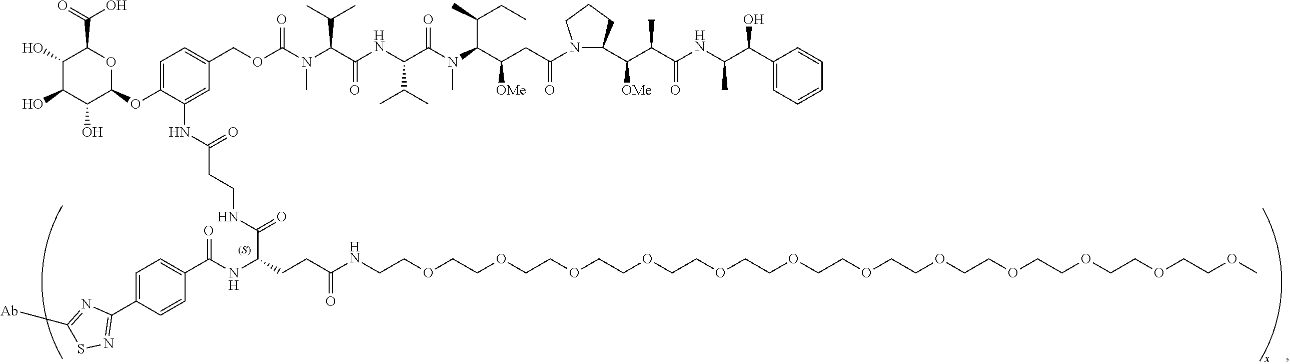

wherein Ab is 6E7 or an antigen-binding fragment thereof;

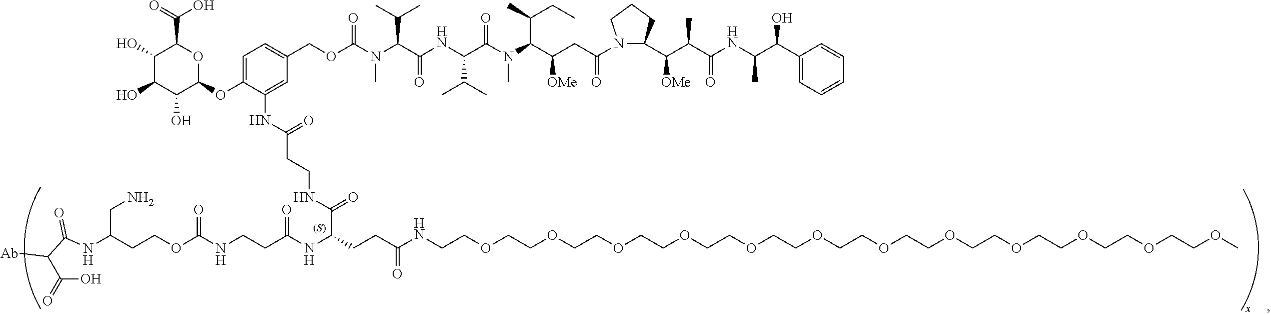

wherein Ab is 6E7 or an antigen-binding fragment thereof;

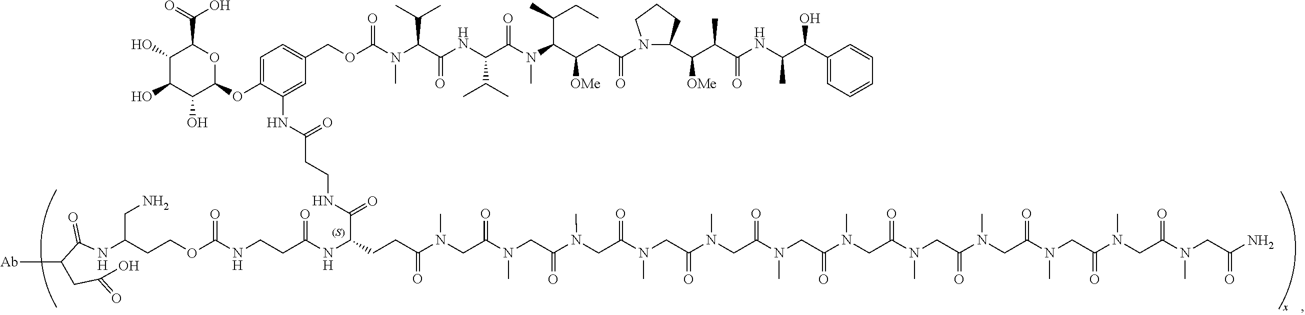

wherein Ab is 6E7 or an antigen-binding fragment thereof;

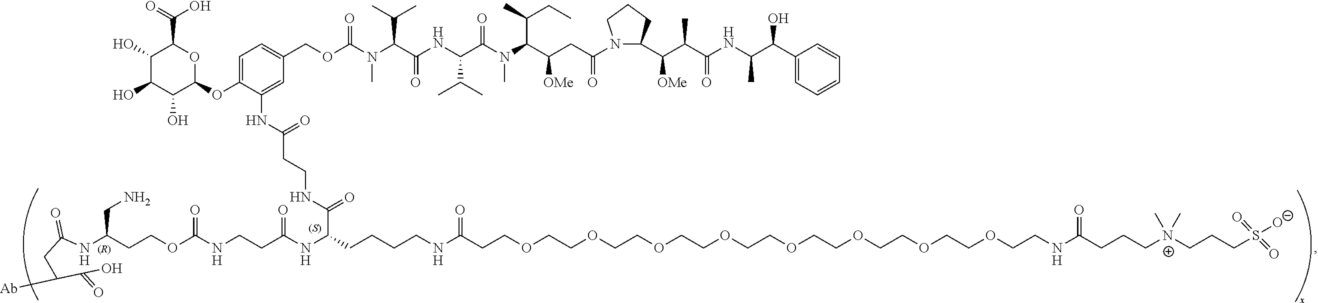

wherein Ab is 6E7 or an antigen-binding fragment thereof;

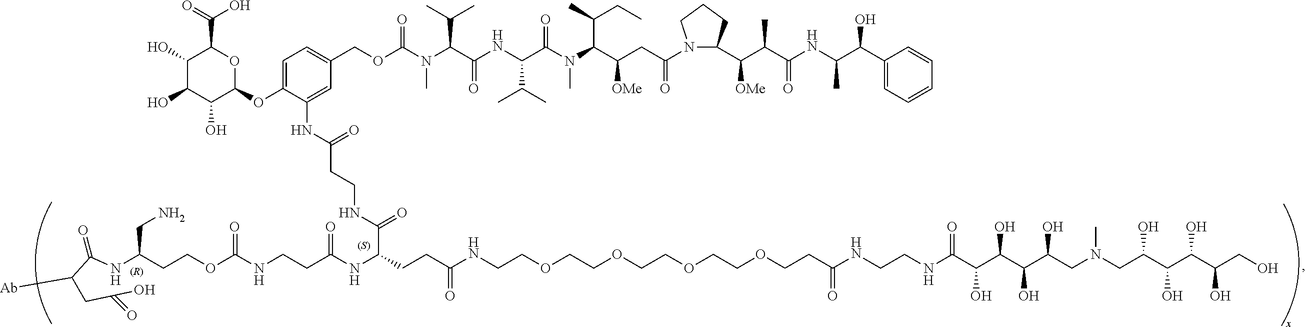

wherein Ab is 6E7 or an antigen-binding fragment thereof;

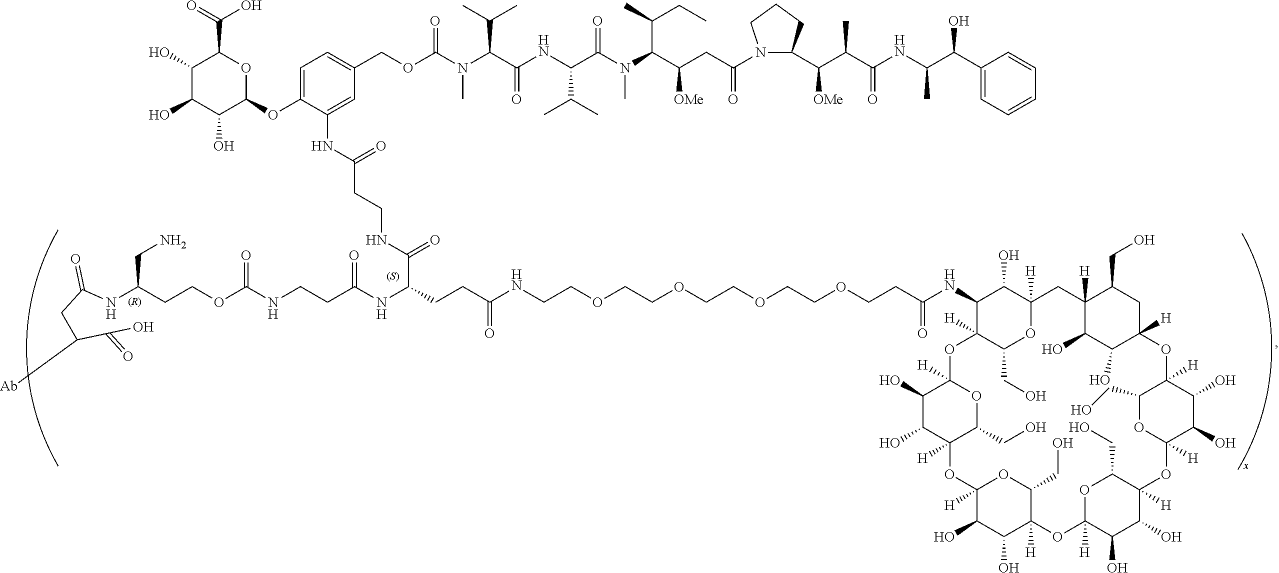

wherein Ab is 6E7 or an antigen-binding fragment thereof;

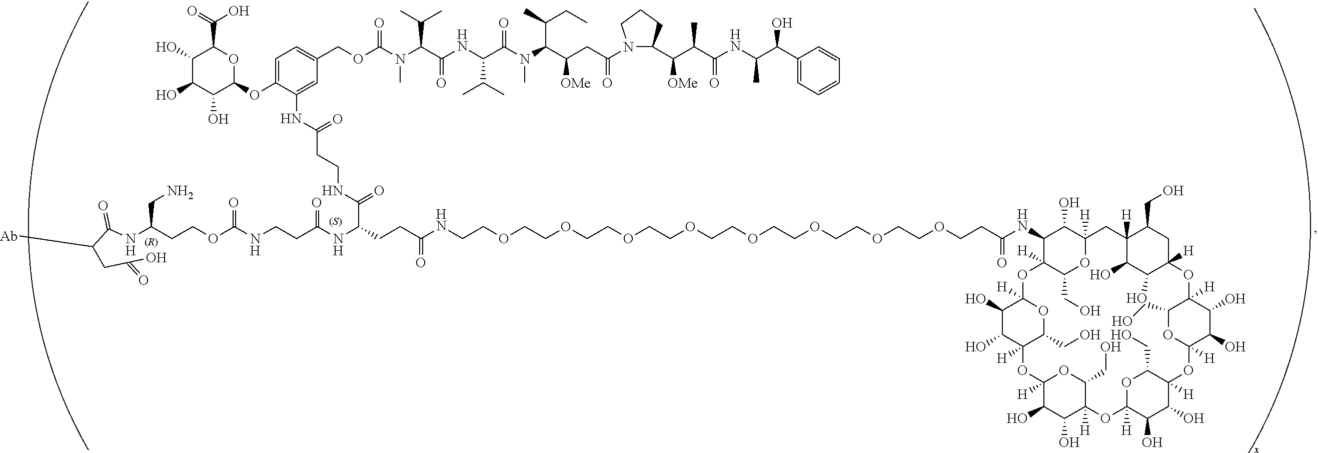

wherein Ab is 6E7 or an antigen-binding fragment thereof;

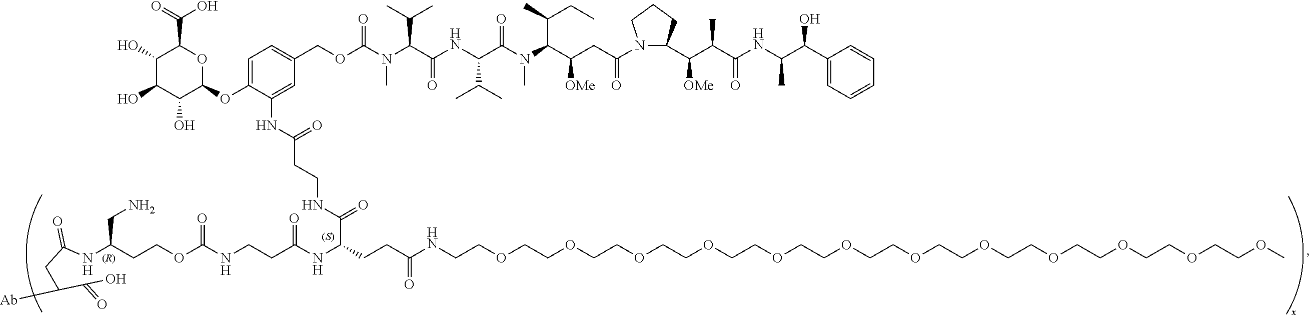

wherein Ab is 6E7 or an antigen-binding fragment thereof;

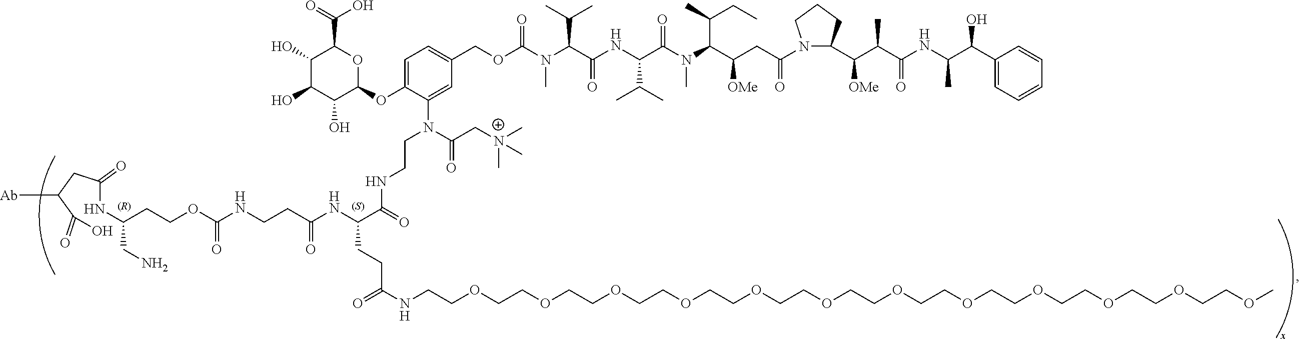

wherein Ab is 6E7 or an antigen-binding fragment thereof;

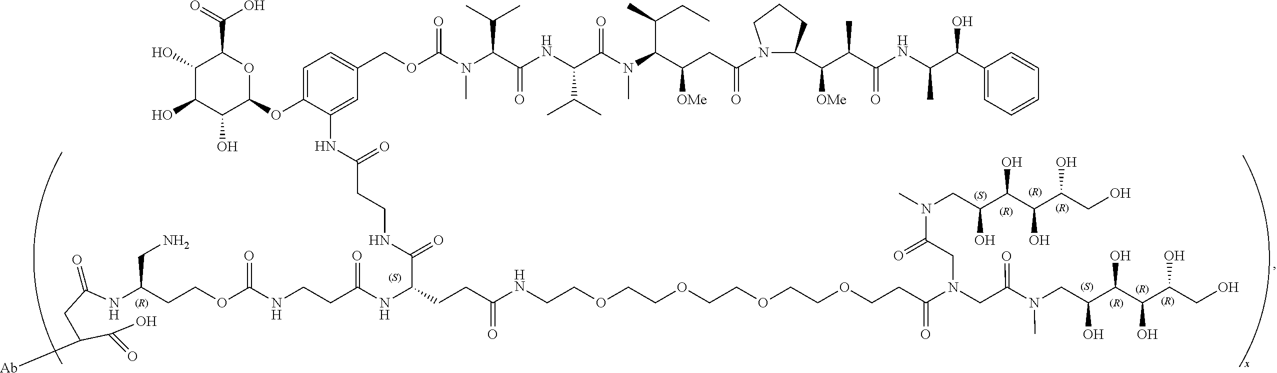

wherein Ab is 6E7 or an antigen-binding fragment thereof;

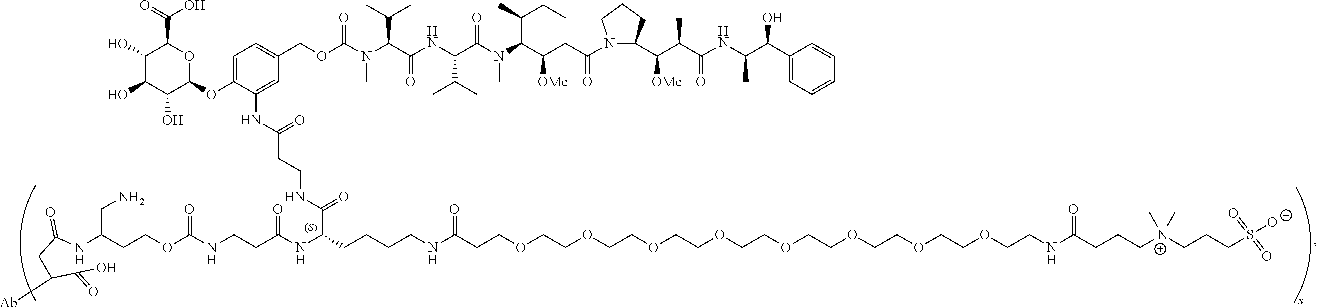

wherein Ab is ifinatamab or an antigen-binding fragment thereof;

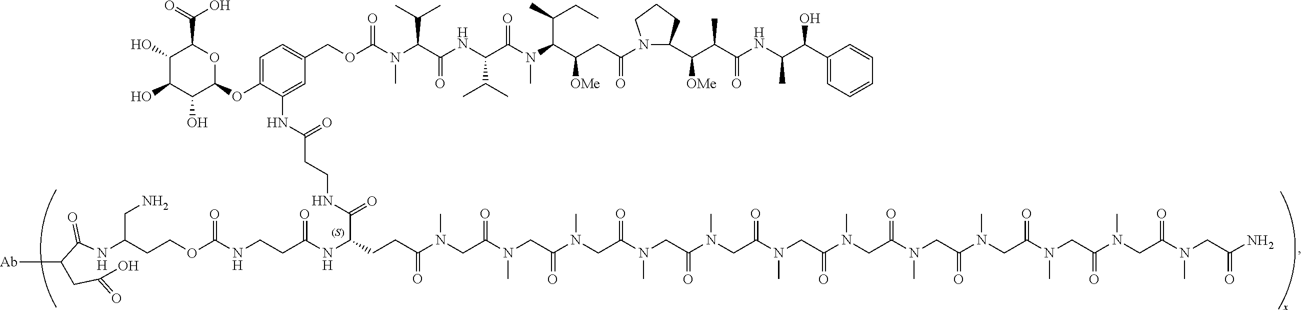

wherein Ab is cofetuzumab or an antigen-binding fragment thereof; or

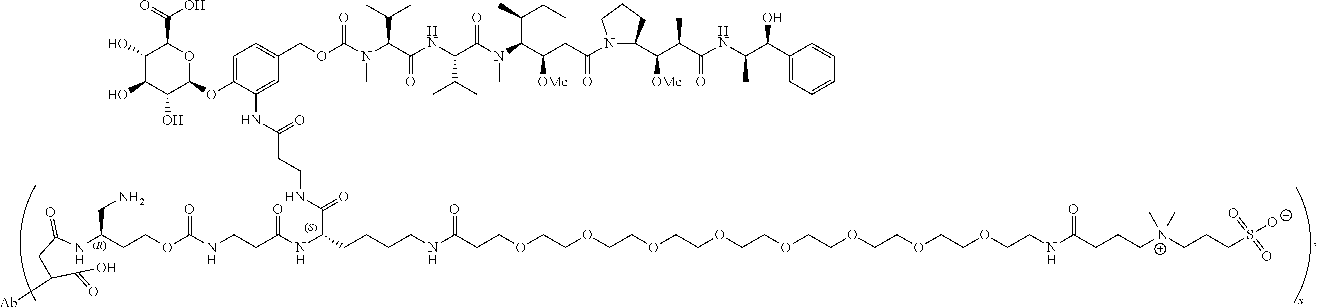

wherein Ab is cofetuzumab or an antigen-binding fragment thereof,
or a pharmaceutically acceptable salt, tautomer, solvate, or stereoisomer of any of the foregoing.

In some embodiments, the compound is

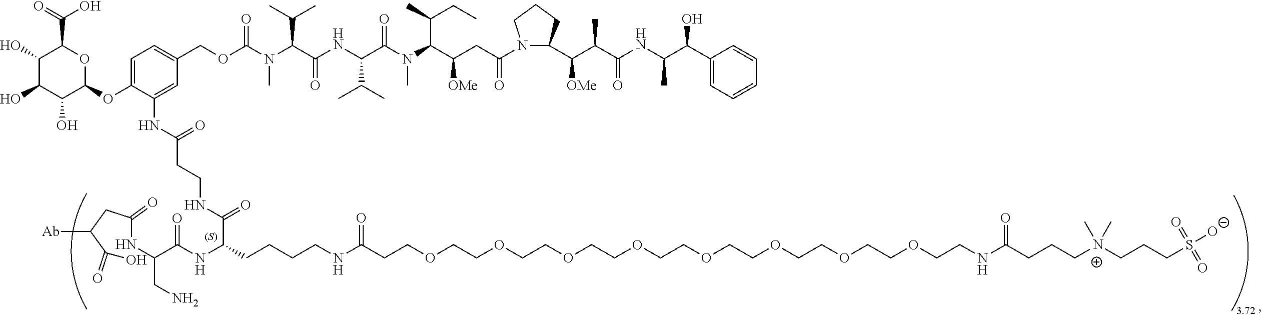

wherein Ab is 6E7 or an antigen-binding fragment thereof;

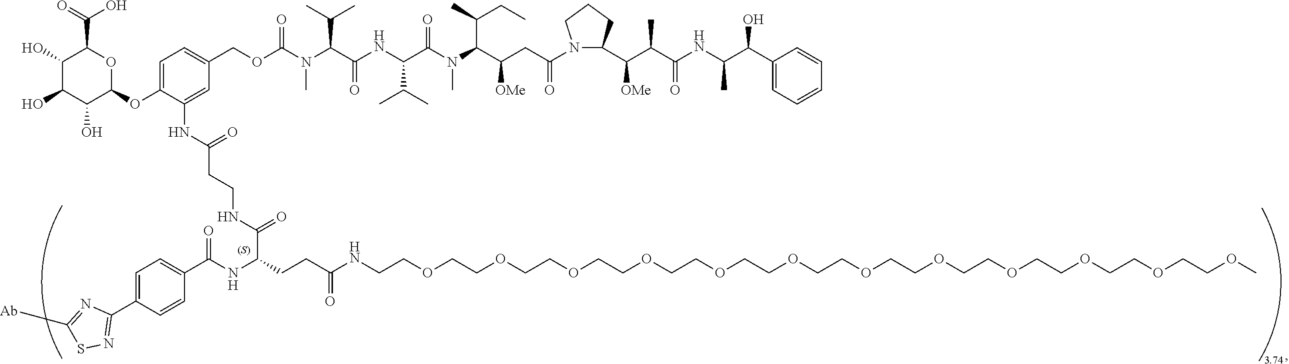

wherein Ab is 6E7 or an antigen-binding fragment thereof;

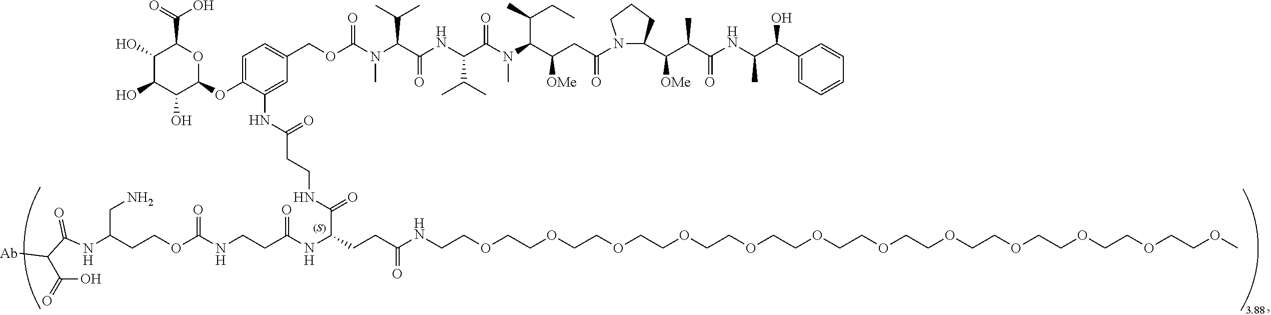

wherein Ab is 6E7 or an antigen-binding fragment thereof;

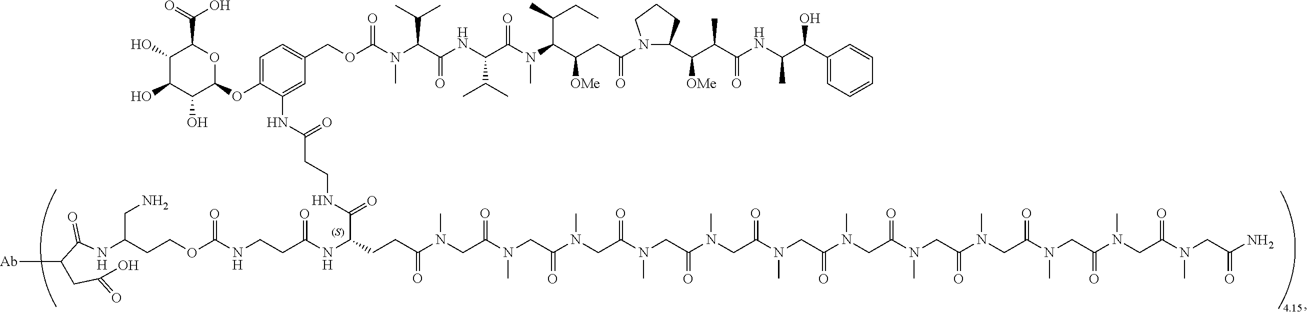

wherein Ab is 6E7 or an antigen-binding fragment thereof;

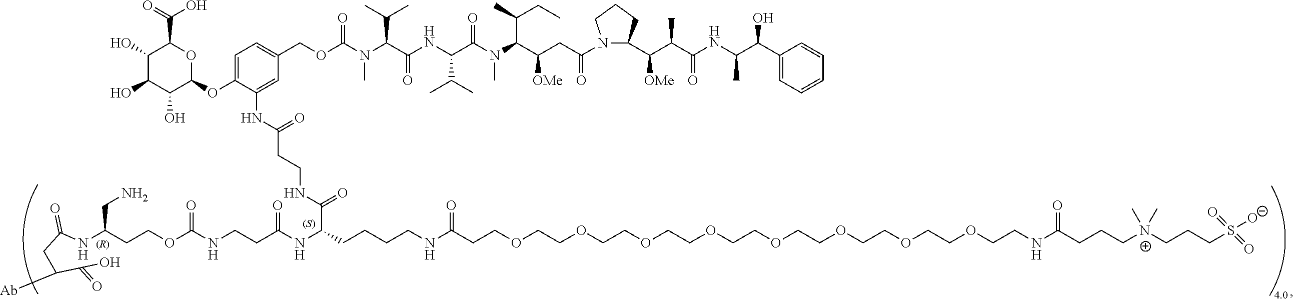

wherein Ab is 6E7 or an antigen-binding fragment thereof;

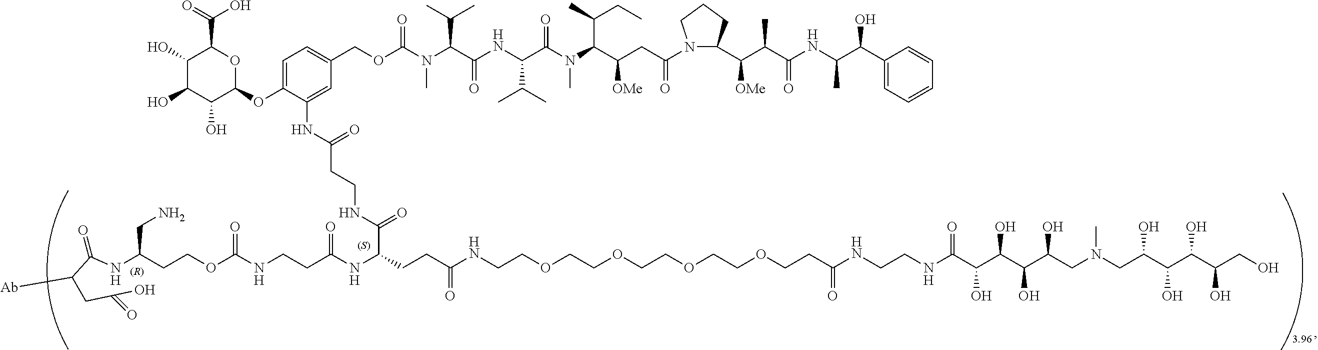

wherein Ab is 6E7 or an antigen-binding fragment thereof;

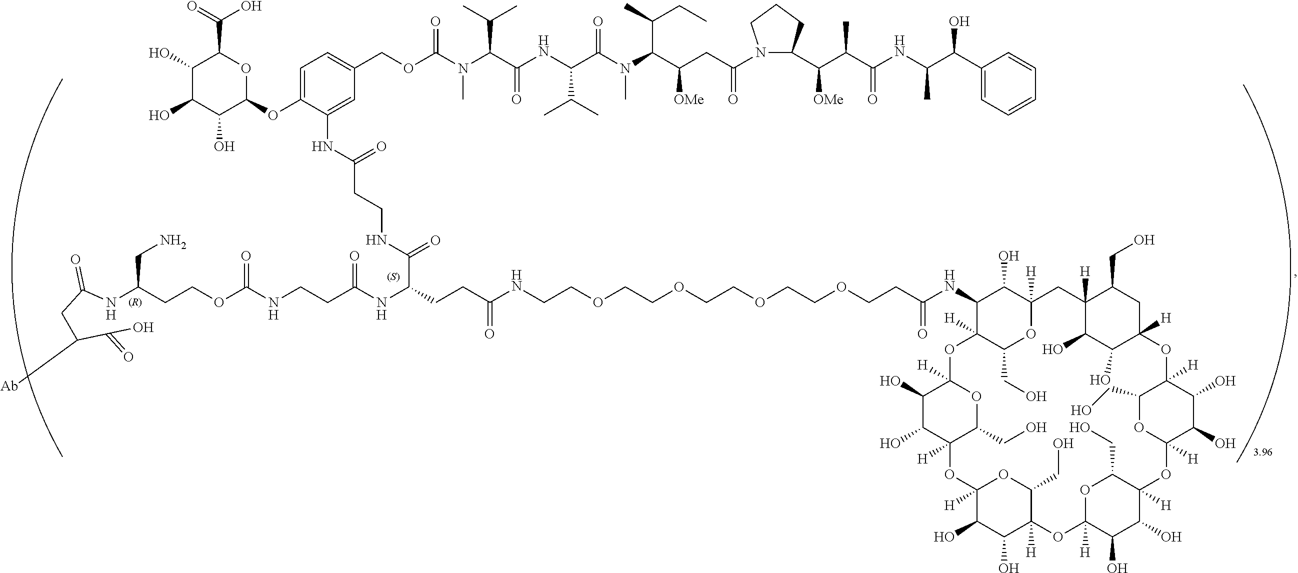

wherein Ab is 6E7 or an antigen-binding fragment thereof;

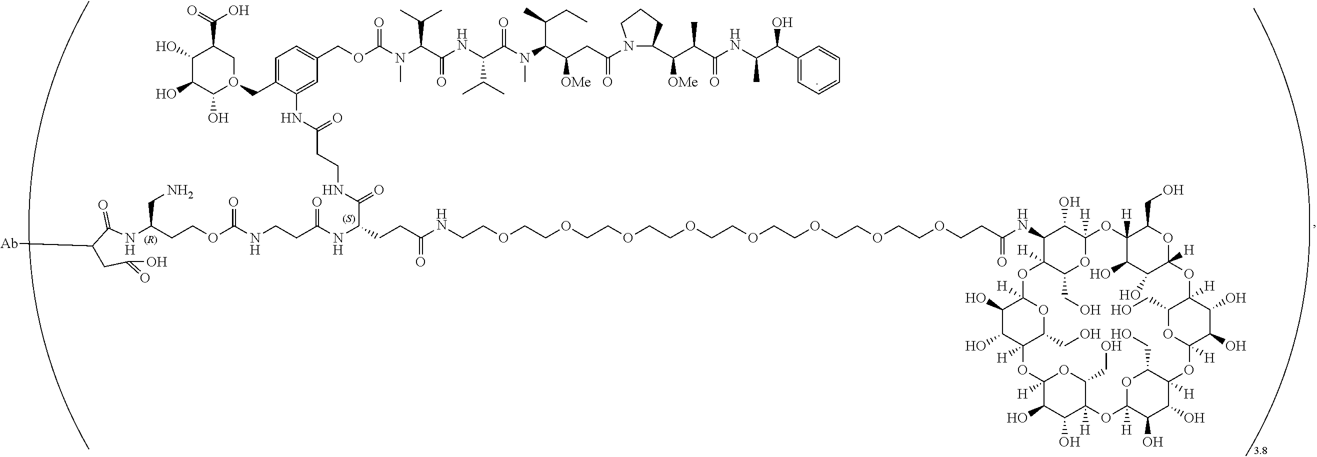

wherein Ab is 6E7 or an antigen-binding fragment thereof;

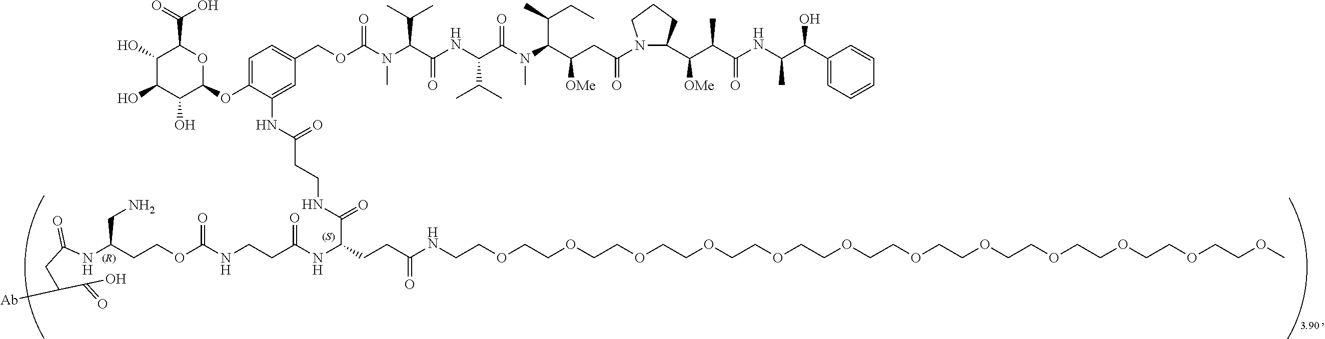

wherein Ab is 6E7 or an antigen-binding fragment thereof;

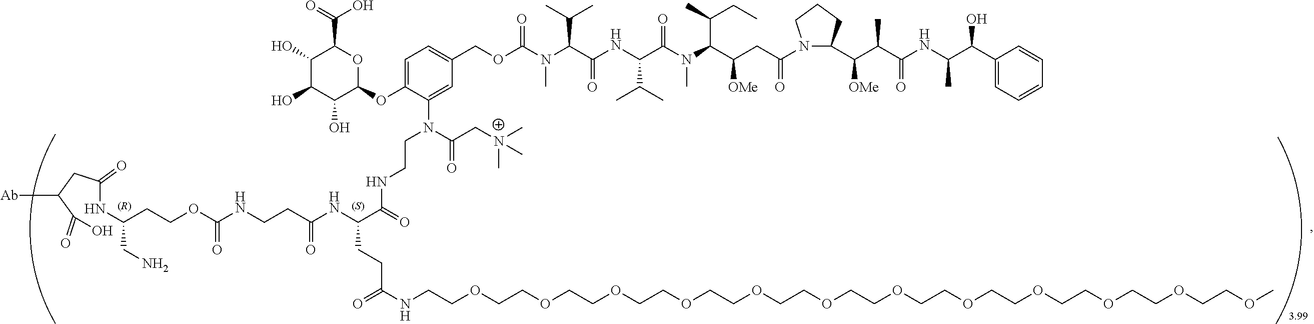

wherein Ab is 6E7 or an antigen-binding fragment thereof;

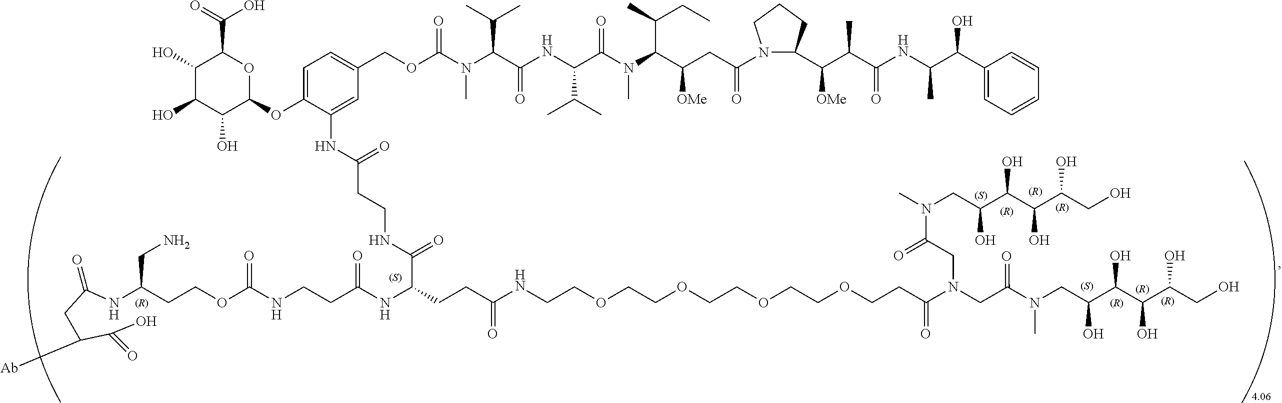

wherein Ab is 6E7 or an antigen-binding fragment thereof;

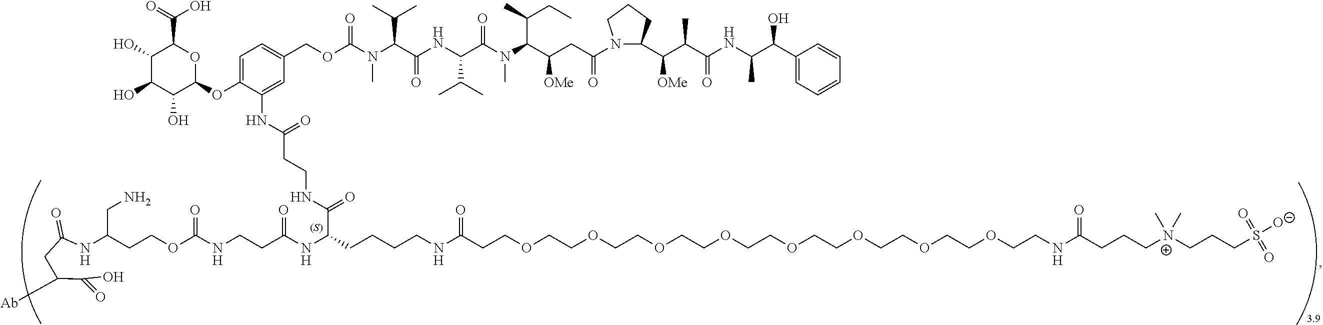

wherein Ab is ifinatamab or an antigen-binding fragment thereof;

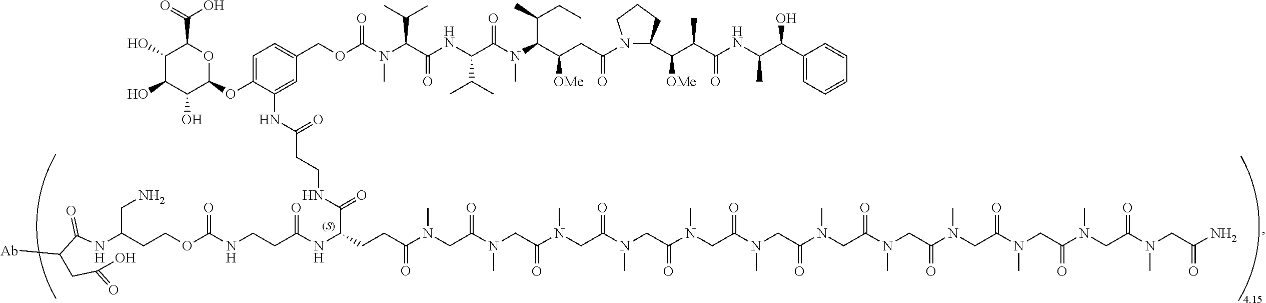

wherein Ab is cofetuzumab or an antigen-binding fragment of thereof; or

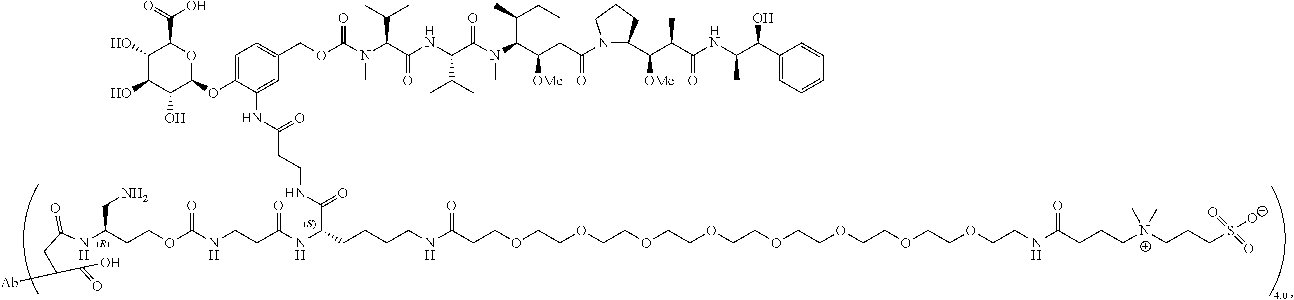

wherein Ab is cofetuzumab or an antigen-binding fragment of thereof,
or a pharmaceutically acceptable salt, tautomer, solvate, or stereoisomer of any of the foregoing.

The binding agent, BA or Ab, may be any BA described above for Aspect 1.

X may be as described above for Aspect 1.

### Aspect 3

Provided herein are a compounds of formula (I-I):

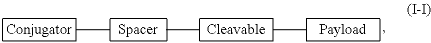

or a pharmaceutically acceptable salt, tautomer, solvate, or stereoisomer thereof, wherein:
Conjugator has formula (I-II) or (I-III):

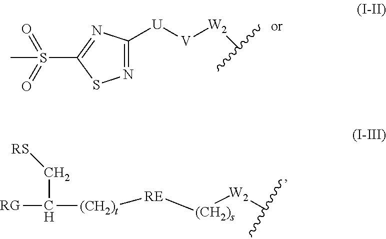

U is a bond, heteroarylene, or arylene;
V is a bond or —C≡C—(CH₂)ₙ—;
n is an integer from 0 to 10 inclusive;
W₂ is —C(═O)—, —NH—, or —O—;
RG is

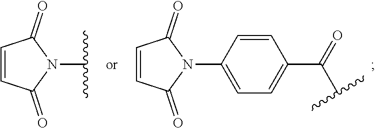 [SMILES: *N1C(=O)C=CC1=O ; *C(=O)c1ccc(N2C(=O)C=CC2=O)cc1]

RS is —NR^{1a}R^{1b};
each of R^{1a} and R^{1b} is, independently, H or substituted or unsubstituted C₁₋₄ alkyl;
RE is a bond, —O—, —OC(═O)—, —OC(═O)NR⁶—, —NHC(═O)NR⁶—, —OS(═O)₂NR⁶—, —NHS(═O)₂NR⁶—, —or —OC(═O)NHS(═O)₂NR⁶—;
R⁶ is H or substituted or unsubstituted C₁₋₄ alkyl;
W₃ is —C(═O)—, —NH—, or —O—;
each of s and t is, independently, 0, 1, or 2;
[symbol] indicates a point of covalent attachment within the compound;
Spacer is a bond, **—NH—(CH₂CH₂O)ₘ—CH₂CH₂—C(═O)—, **—(CH₂)ₘ—C(═O)—, **—CH₂—C(═O)—NH—(CH₂)ₘ—C(═O)—, **—(CH₂CH₂O)ₘ—CH₂CH₂—C(═O)—, *—CH[—(CH₂)ₘ—COOH]—C(═O)—, **—CH₂—C(═O)—NH—(CH₂)ₘ—C(═O)—NH—(CH₂)ₘ—C(═O)—, **—C(═O)—(CH₂)ₘ—C(═O)—, **—NH—(CH₂)ₘ—C(═O)—, or **—NH—(CH₂)ₘ—O—;
m is an integer from 1 to 12 inclusive;
* marks the bond where Spacer connects to Conjugator;
Cleavable has formula (IVa), (IVb), (IVc), (Va), (Vb), (VIa), (VIb), (VIIa), or (VIIb):

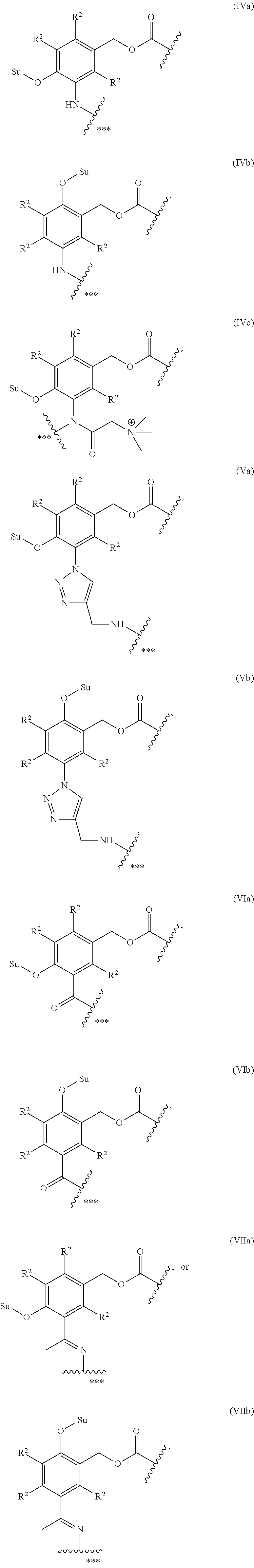

Su is a Sugar moiety;
each R² is, independently, hydrogen, halogen, substituted or unsubstituted C₁₋₄ alkyl, —CN, or —NO₂;
*** marks the bond where Cleavable connects to Spacer; and
Payload is a payload residue.

In some embodiments, Conjugator has formula (I-II).

In some embodiments, U is arylene.

In some embodiments, U is phenylene.

In some embodiments, U is

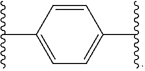 [SMILES: *c1ccc(*)cc1]

In some embodiments, U is arylene and V is a bond.

In some embodiments, U is arylene and V is —C≡C—(CH₂)ₙ—.

In some embodiments, U is arylene and V is —C≡C—(CH₂)₃—.

In some embodiments, U is heteroarylene.

In some embodiments, U is a bivalent pyrimidine ring.

In some embodiments, U is

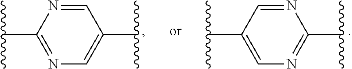 [SMILES: *c1cnc(*)nc1 ; *c1cnc(*)nc1]

In some embodiments, U is heteroarylene and V is a bond.

In some embodiments, U is heteroarylene and V is —C≡C—(CH₂)ₙ—.

In some embodiments, U is heteroarylene and V is —C≡C—(CH₂)₃—.

In some embodiments, U is a bond.

In some embodiments, U is a bond and V is a bond.

In some embodiments, U is a bond and V is —C≡C—(CH₂)ₙ—.

In some embodiments, U is a bond and V is —C≡C—(CH₂)₃—.

In some embodiments, W₂ is —C(═O)—.

In some embodiments, Conjugator has formula (I-III).

In some embodiments, RS is —NH₂ or —N(CH₃)₂.

In some embodiments, RS is —NH₂.

In some embodiments, RE is —OC(═O) NH—.

In some embodiments, RG is

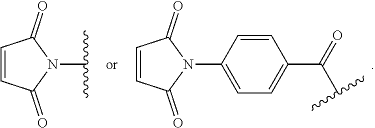 [SMILES: *N1C(=O)C=CC1=O ; *C(=O)c1ccc(N2C(=O)C=CC2=O)cc1]

In some embodiments, RG is

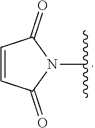 [SMILES: *N1C(=O)C=CC1=O]

In some embodiments, each of s and t is 2.

In some embodiments, W₃ is —C(═O)— or —NH—.

In some embodiments, the Spacer is a bond, **—NH—(CH₂CH₂O)ₘ—CH₂CH₂—C(═O)—, or **—C(═O)—(CH₂CH₂O)ₘ—CH₂CH₂—NH—.

In some embodiments, m is 2, 4, 6, or 8.

In some embodiments, Conjugator has (I-IIa1) or (I-IIIa1):

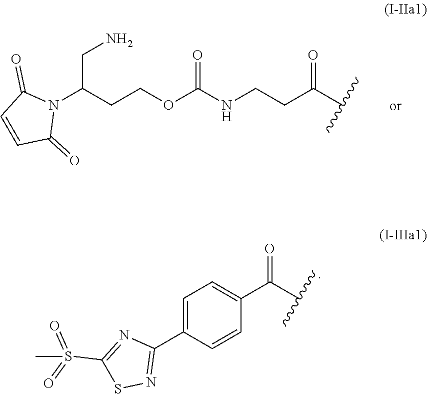 [SMILES: *C(=O)CCNC(=O)OCCC(CN)N1C(=O)C=CC1=O ; *C(=O)c1ccc(-c2nsc(S(C)(=O)=O)n2)cc1]

In some embodiments, Cleavable has one of the following formulas:

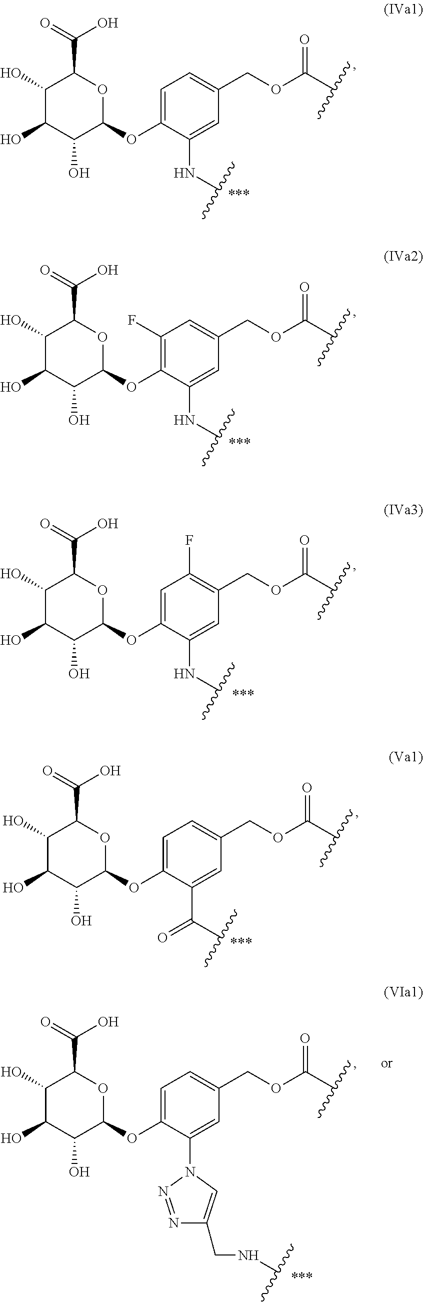 [SMILES: *Nc1cc(COC(*)=O)ccc1O[C@@H]1O[C@H](C(=O)O)[C@@H](O)[C@H](O)[C@H]1O ; *Nc1cc(COC(*)=O)cc(F)c1O[C@@H]1O[C@H](C(=O)O)[C@@H](O)[C@H](O)[C@H]1O ; *Nc1cc(COC(*)=O)c(F)cc1O[C@@H]1O[C@H](C(=O)O)[C@@H](O)[C@H](O)[C@H]1O ; *C(=O)OCc1ccc(O[C@@H]2O[C@H](C(=O)O)[C@@H](O)[C@H](O)[C@H]2O)c(C(*)=O)c1 ; *NCc1cn(-c2cc(COC(*)=O)ccc2O[C@@H]2O[C@H](C(=O)O)[C@@H](O)[C@H](O)[C@H]2O)nn1]

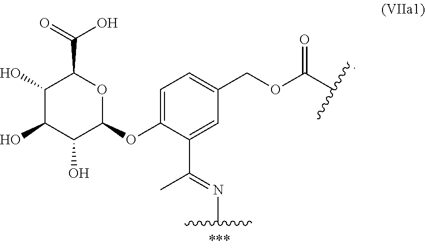 [SMILES: */N=C(\C)c1cc(COC(*)=O)ccc1O[C@@H]1O[C@H](C(=O)O)[C@@H](O)[C@H](O)[C@H]1O]

In some embodiments, Payload is a residue of:

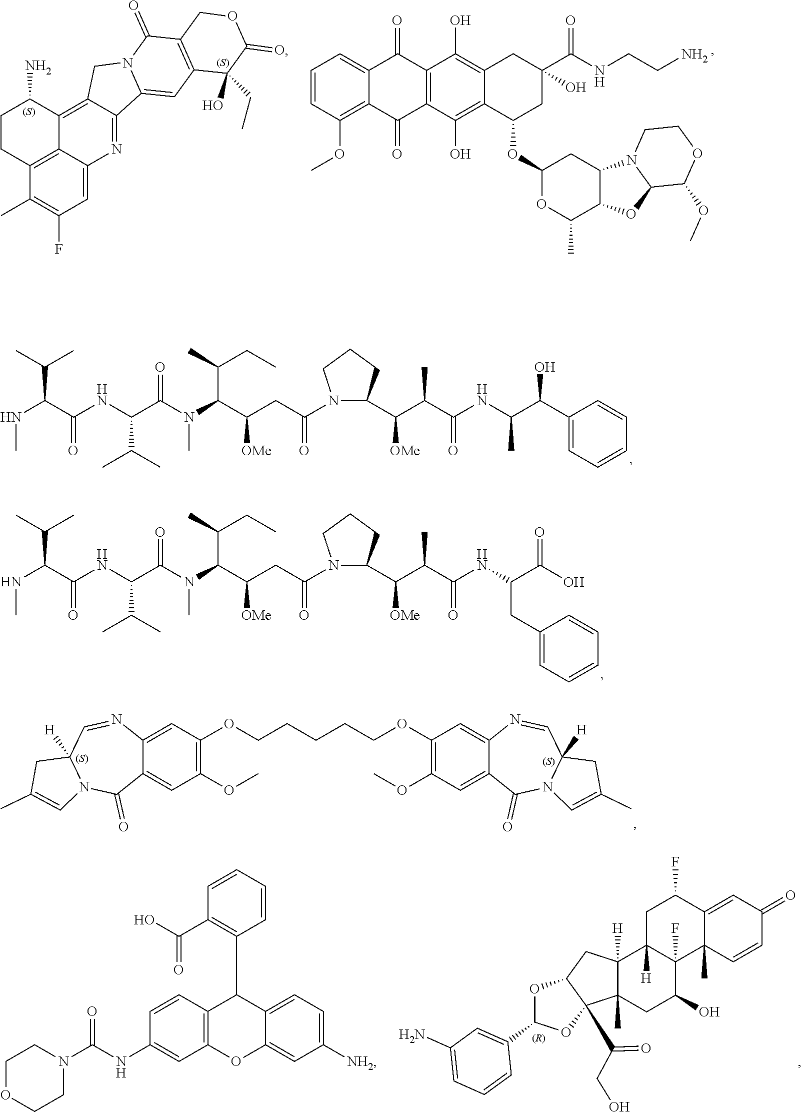

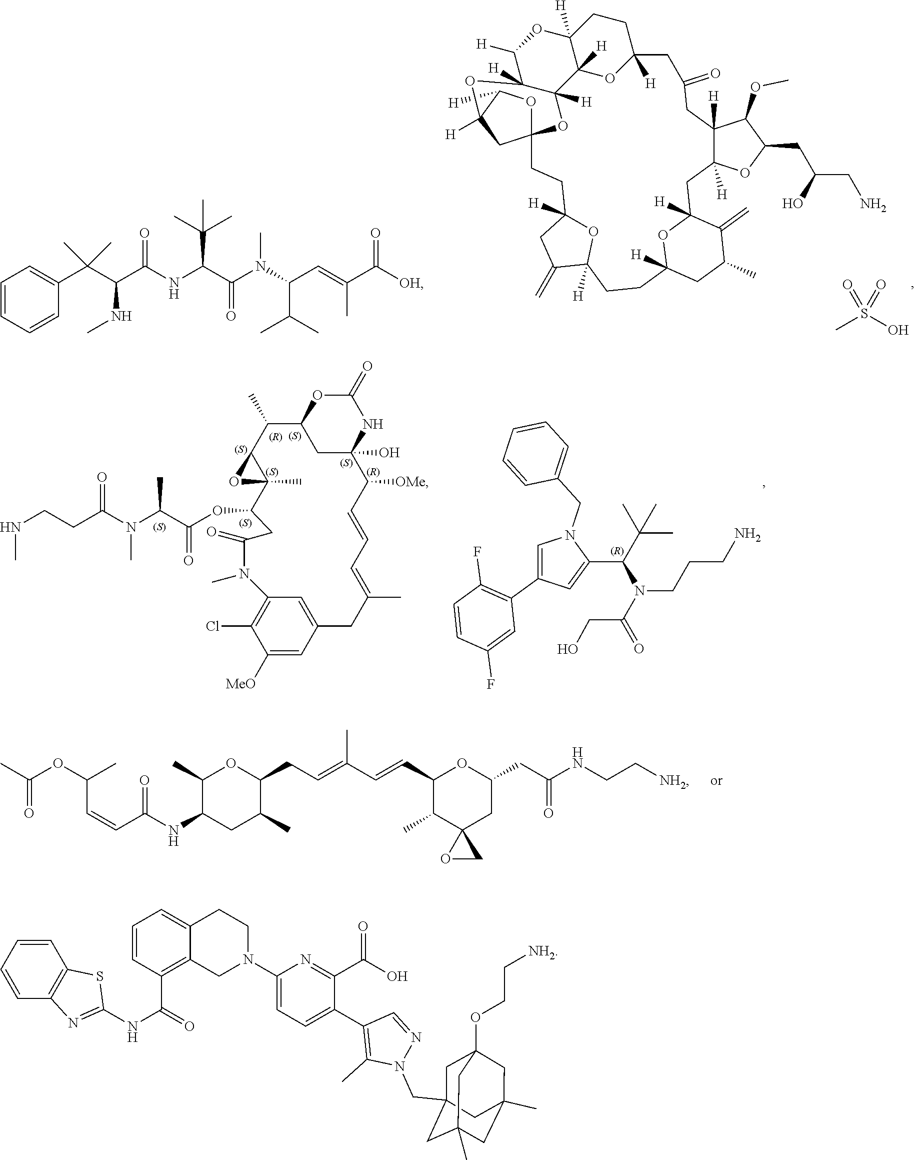

In some embodiments, Payload is

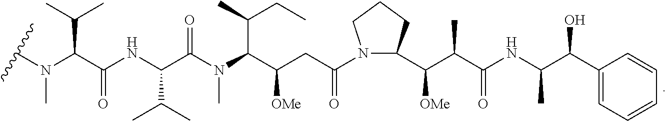 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

In some embodiments, the compound is

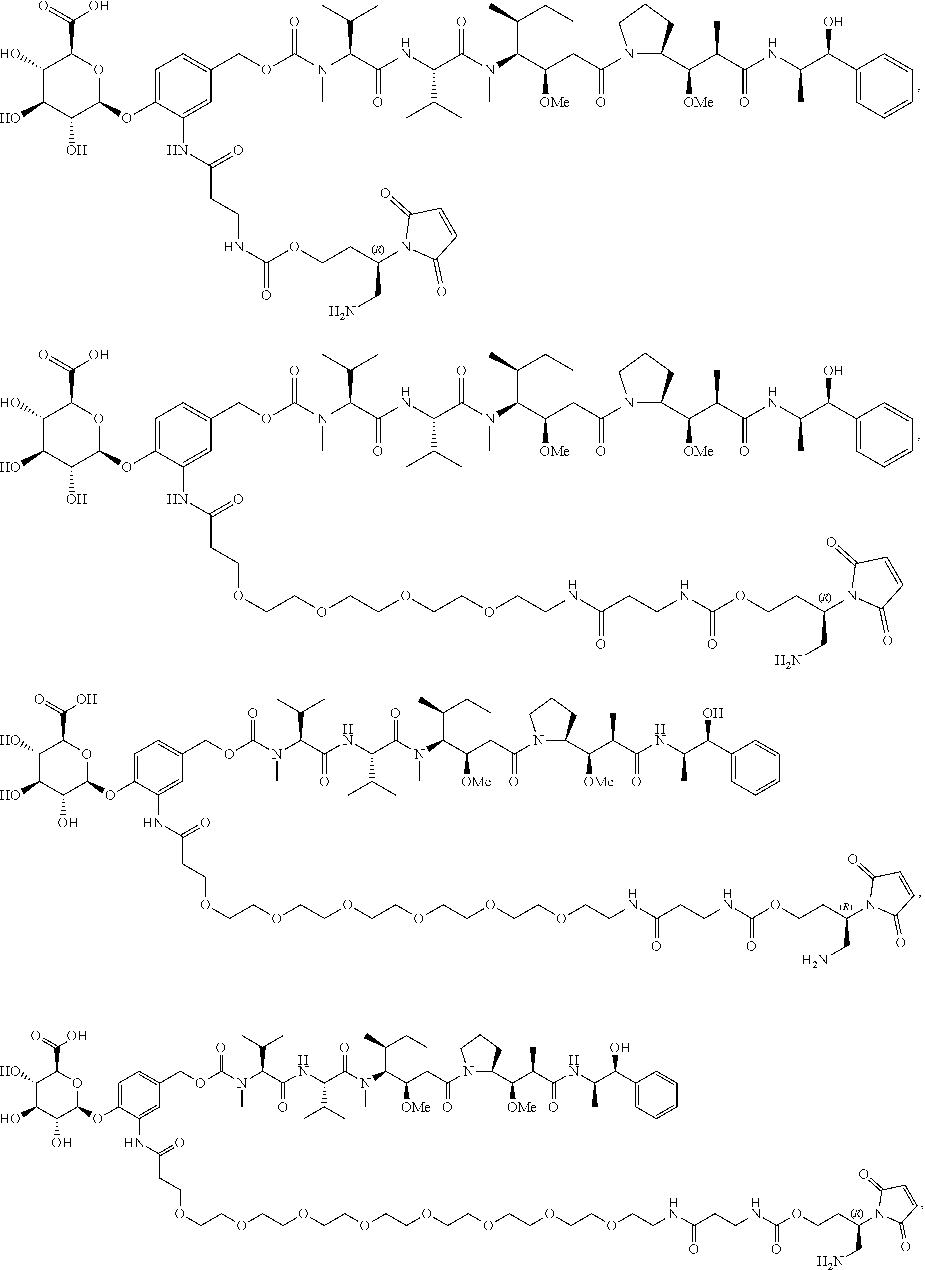

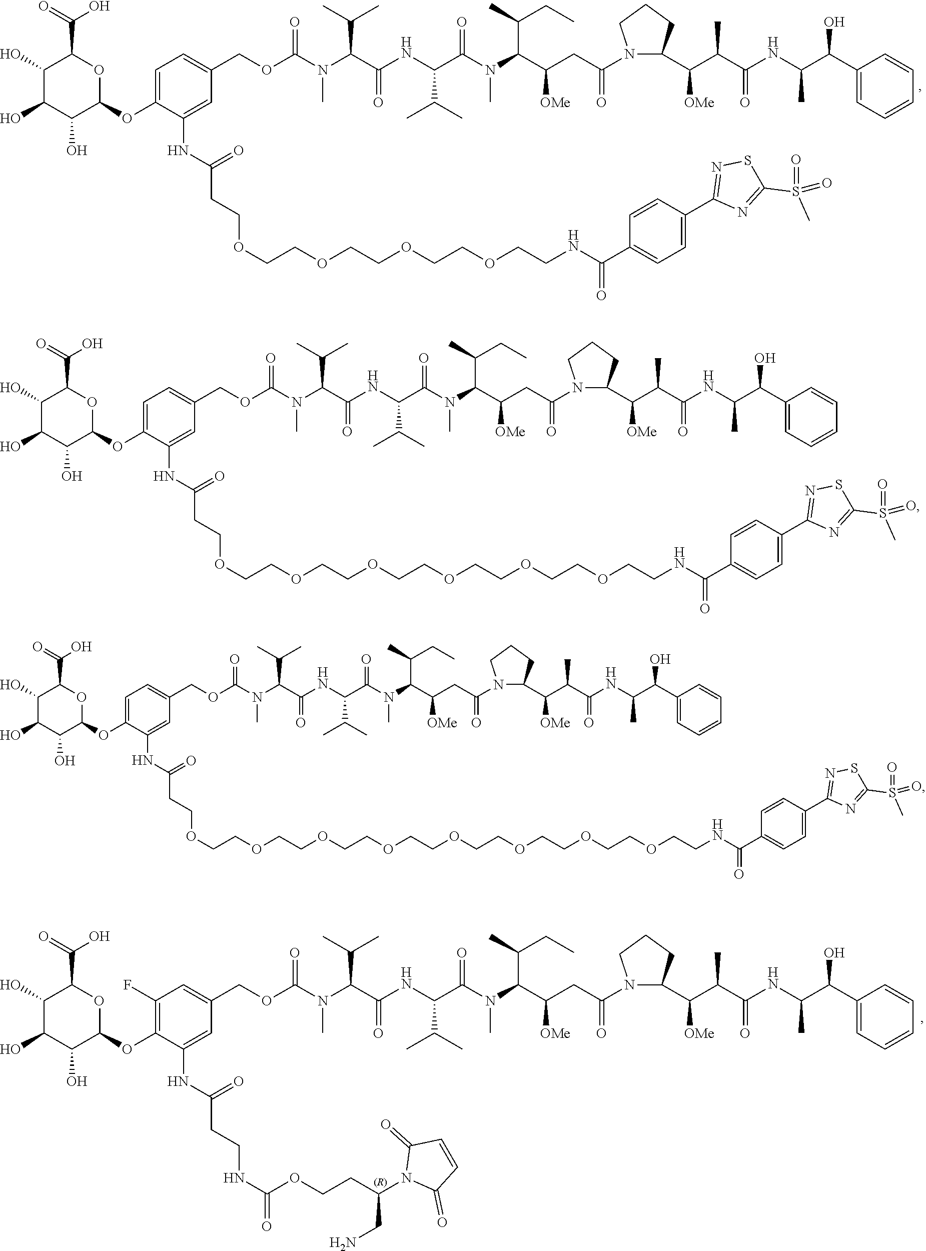

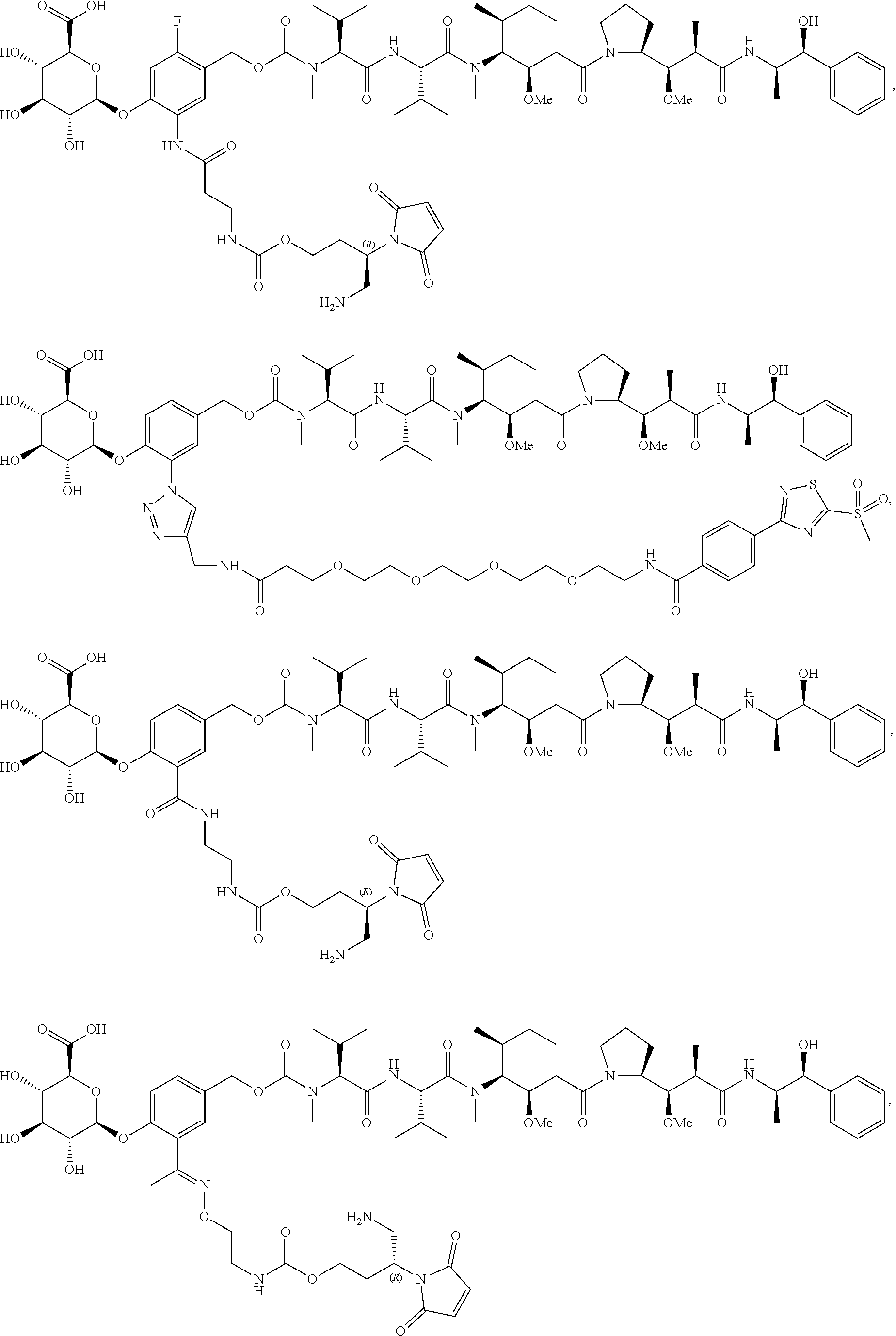

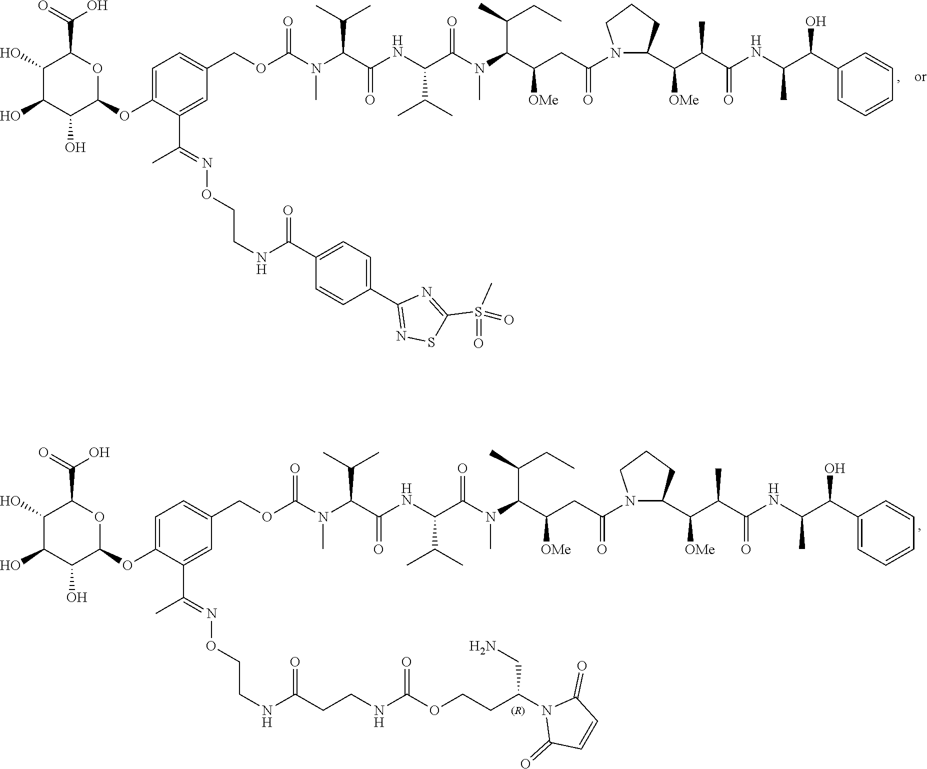

or a pharmaceutically acceptable salt, tautomer, solvate, or stereoisomer thereof.

### Aspect 4

Provided herein are compounds of formula (XV):

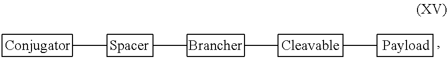

or a pharmaceutically acceptable salt, tautomer, solvate, or stereoisomer thereof, wherein:
Conjugator has formula (I-II) or (I-III):

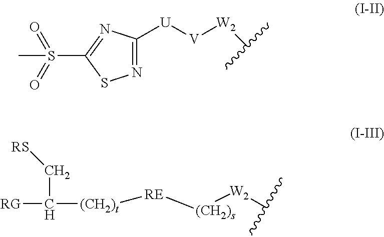

U is a bond, heteroarylene, or arylene;
V is a bond or —C≡C—(CH₂)ₙ—;
n is an integer from 0 to 10 inclusive;
W₂ is —C(═O)—, —NH—, or —O—;
RG is

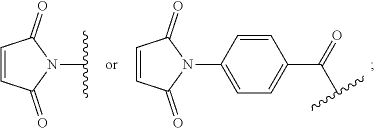 [SMILES: *N1C(=O)C=CC1=O ; *C(=O)c1ccc(N2C(=O)C=CC2=O)cc1]

RS is —NR^{1a}R^{1b};
each of R^{1a} and R^{1b} is, independently, H or substituted or unsubstituted C₁₋₄ alkyl;
RE is a bond, —O—, —OC(═O)—, —OC(═O)NR⁶—, —NHC(═O)NR⁶—, —OS(═O)₂NR⁶—, —NHS(═O)₂NR⁶—, —or —OC(═O)NHS(═O)₂NR⁶—;
R⁶ is H or substituted or unsubstituted C₁₋₄ alkyl;
W₃ is —C(═O)—, —NH—, or —O—;
each of s and t is, independently, 0, 1, or 2;
[symbol] indicates a point of covalent attachment within the compound;
Spacer is a bond, **—NH—(CH₂CH₂O)ₘ—CH₂CH₂—C(═O)—, **—(CH₂)ₘ—C(═O)—, *—CH₂—C(═O)—NH—(CH₂)ₘ—C(═O)—, **—(CH₂CH₂O)ₘ—CH₂CH₂—C(═O)—, **—CH[—(CH₂)ₘ—COOH]—C(═O)—, **—CH₂—C(═O)—NH—(CH₂)ₘ—C(═O)—NH—(CH₂)ₘ—C(═O)—, **—C(═O)—(CH₂)ₘ—C(═O)—, **—NH—(CH₂)ₘ—C(═O)—, or **—NH—(CH₂)ₘ—O—;
m is an integer from 1 to 12 inclusive;
** marks the bond where Spacer connects to Conjugator;
Cleavable has formula (IVa′), (IVb′), (IVc′), (Va′), (Vb′), (VIa′), (VIb′), (VIIa′), or (VIIb′):

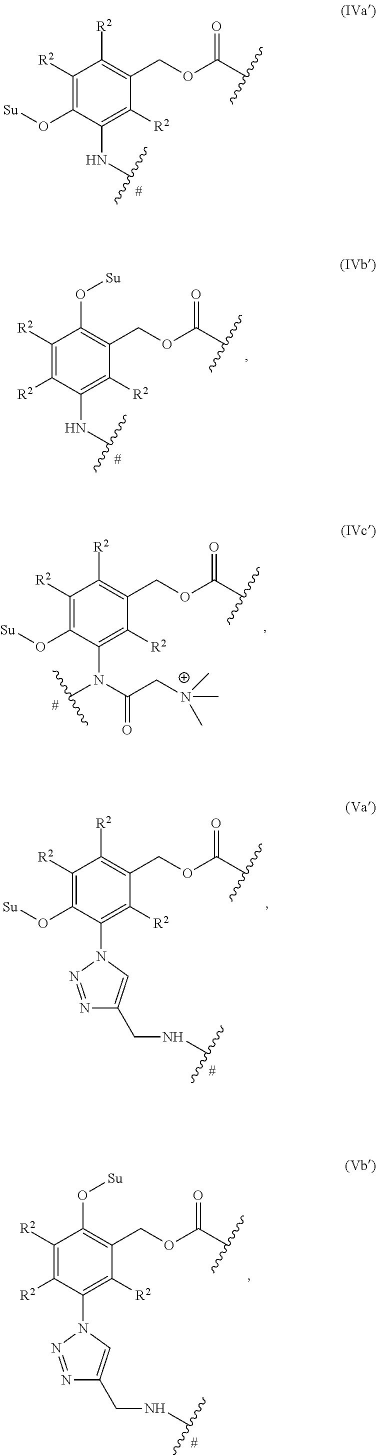

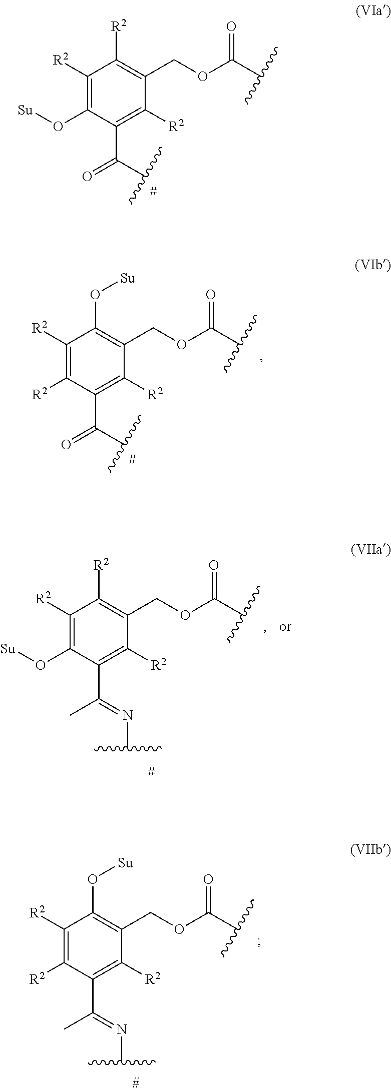

Su is a Sugar moiety;
each R² is, independently, hydrogen, halogen, substituted or unsubstituted C₁₋₄alkyl, —CN, or —NO₂; #marks the bond where Cleavable connects to Brancher;
Brancher is selected from formula (XIIa), (XIIb), (XIIc), and (XIId):

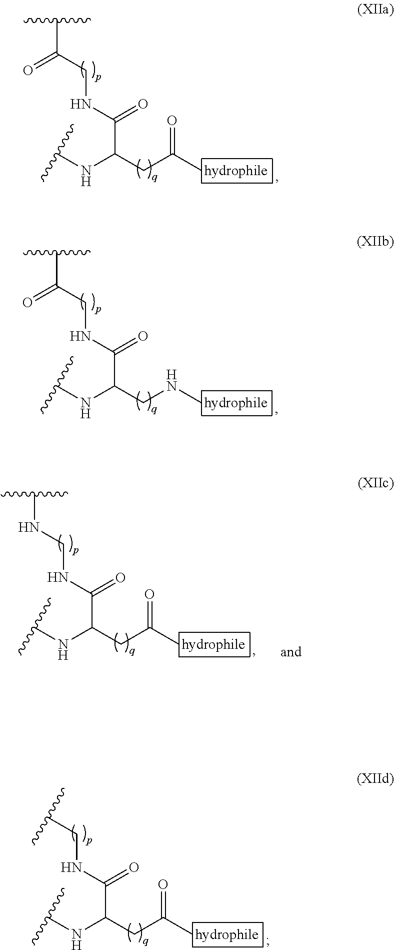

each of p and q is, independently, 1, 2, 3, or 4;
hydrophile is —NH—(CH₂CH₂O)ₐ—(CH₂)_{b}CH₃, —N(CH₃)—((CH₂)_{c}C(═O) N(CH₃))ₐ—(CH₂)_{c} C(═O) NH₂, —C(═O)—(CH₂CH₂O)ₐ—(CH₂)_{b}CH₃, —C(═O)—(CH₂CH₂O)ₐ—(CH₂)_{b}NHC(═O) (CH₂)₃N(CH₃)₂ (CH₂)₃S(═O)₂OH, —NH—(CH₂CH₂O)ₐ—(CH₂)_{b}NHC(═O) (CH₂)₃N(CH₃)₂ (CH₂)₃S(═O)₂OH,

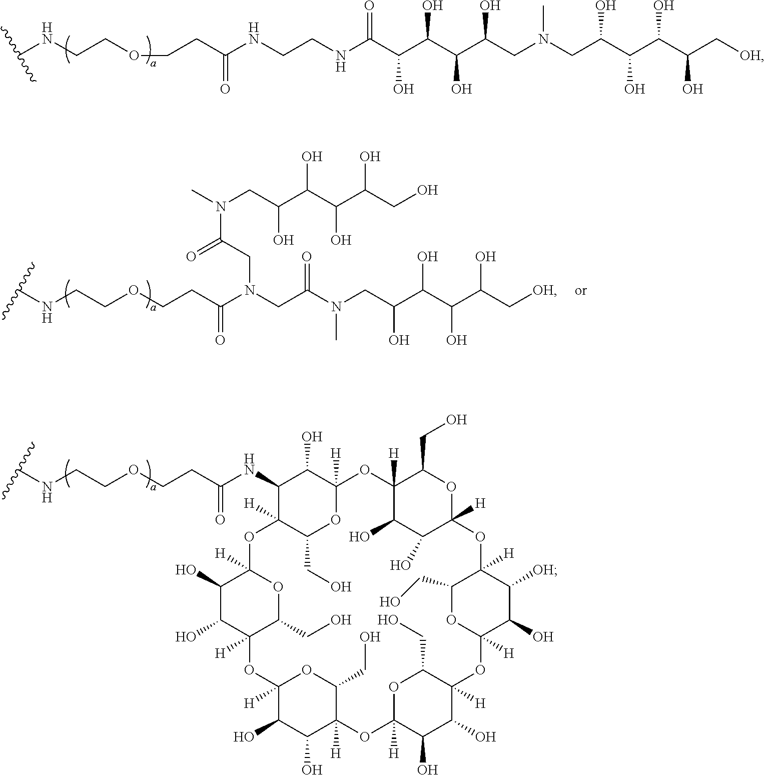

a is an integer from 1 to 18 inclusive;
b is 0, 1, or 2;
c is 1, 2, 3, or 4; and
Payload is a payload residue.

In some embodiments, Conjugator has formula (I-II).

In some embodiments, U is arylene.

In some embodiments, U is phenylene.

In some embodiments, U is

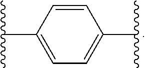 [SMILES: *c1ccc(*)cc1]

In some embodiments, U is arylene and V is a bond.

In some embodiments, U is arylene and V is —C≡C—(CH₂)ₙ—.

In some embodiments, U is arylene and V is —C≡C—(CH₂)₃—.

In some embodiments, U is heteroarylene.

In some embodiments, U is a bivalent pyrimidine ring.

In some embodiments, U is

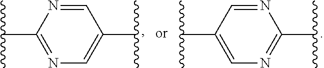

In some embodiments, U is heteoarylene and V is a bond.

In some embodiments, U is heteoarylene and V is —C≡C—(CH₂)ₙ—.

In some embodiments, U is heteoarylene and V is —C≡C—(CH₂)₃—.

In some embodiments, U is a bond.

In some embodiments, U is a bond and V is a bond.

In some embodiments, U is a bond and V is —C≡C—(CH₂)ₙ—.

In some embodiments, U is a bond and V is —C≡C—(CH₂)₃—.

In some embodiments, W₂ is —C(═O)—.

In some embodiments, Conjugator has formula (I-III).

In some embodiments, RS is —NH₂ or —N(CH₃)₂.

In some embodiments, RS is —NH₂.

In some embodiments, RE is —OC(═O) NH—.

In some embodiments, RG is

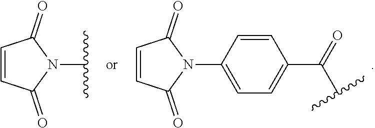 [SMILES: *N1C(=O)C=CC1=O ; *C(=O)c1ccc(N2C(=O)C=CC2=O)cc1]

In some embodiments, RG is

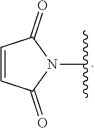 [SMILES: *N1C(=O)C=CC1=O]

In some embodiments, each of s and t is 2.

In some embodiments, Spacer is a bond, **—NH—(CH₂CH₂O)ₘ—CH₂CH₂—C(═O)—, **—C(═O)—(CH₂CH₂O)ₘ—CH₂CH₂—NH—, or **—NH—(CH₂)ₘ—O—. In some embodiments, Spacer is a bond, **—NH—(CH₂CH₂O)ₘ—CH₂CH₂—C(═O)—, or **—NH—(CH₂)ₘ—O—.

In some embodiments, m is 2, 4, 6, or 8.

In some embodiments, Conjugator has formula (I-IIa1) or (I-IIIa1):

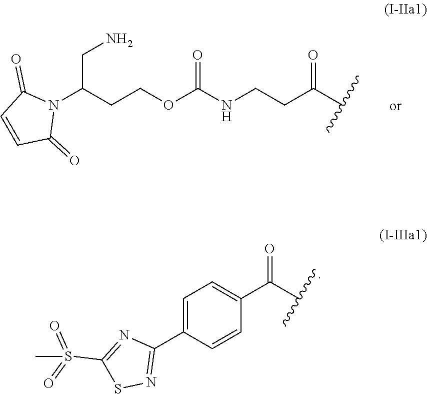 [SMILES: *C(=O)CCNC(=O)OCCC(CN)N1C(=O)C=CC1=O ; *C(=O)c1ccc(-c2nsc(S(C)(=O)=O)n2)cc1]

In some embodiments, Cleavable has one of the following formulas:

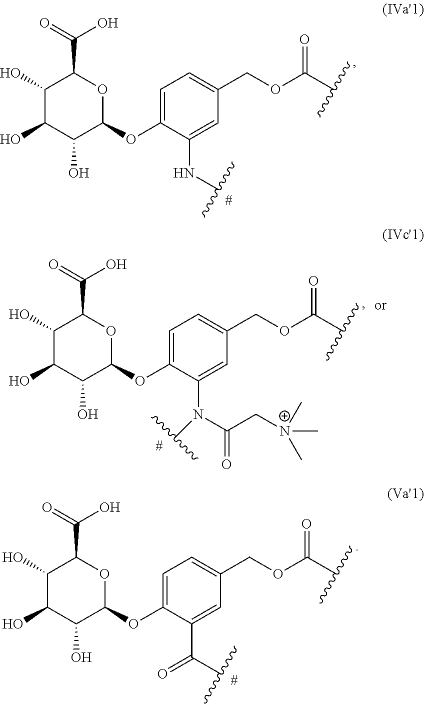 [SMILES: *Nc1cc(COC(*)=O)ccc1O[C@@H]1O[C@H](C(=O)O)[C@@H](O)[C@H](O)[C@H]1O ; *C(=O)OCc1ccc(O[C@@H]2O[C@H](C(=O)O)[C@@H](O)[C@H](O)[C@H]2O)c(N(*)C(=O)C[N+](C)(C)C)c1 ; *C(=O)OCc1ccc(O[C@@H]2O[C@H](C(=O)O)[C@@H](O)[C@H](O)[C@H]2O)c(C(*)=O)c1]

In some embodiments, Payload is a residue of:

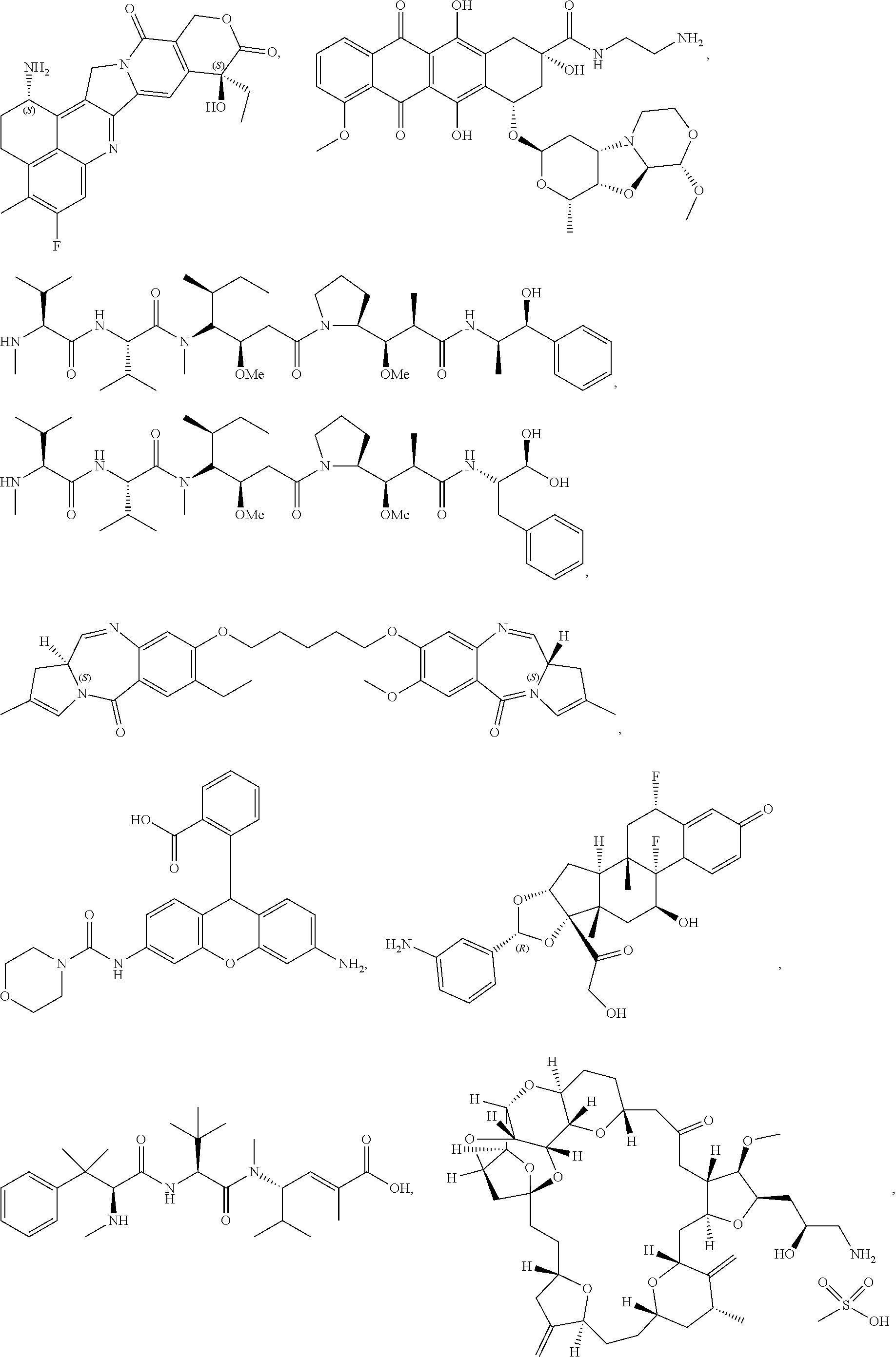

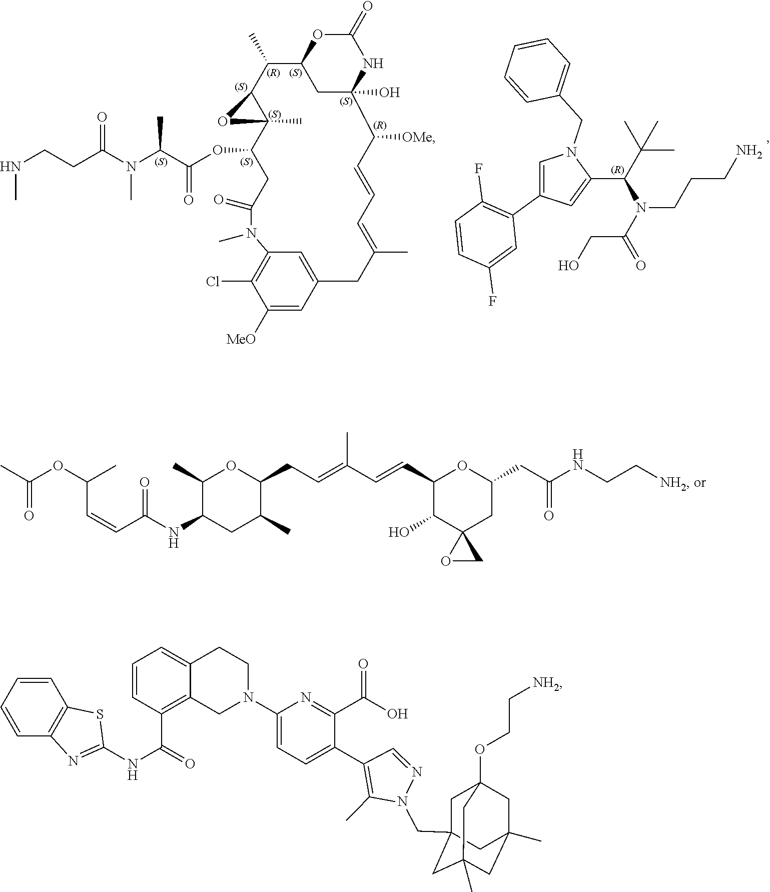

In some embodiments, Payload is

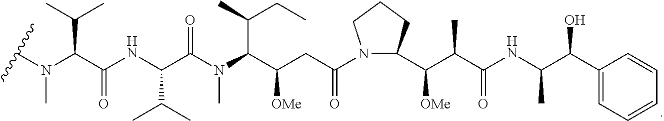 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

In some embodiments, a is 12 and b is 0.

In some embodiments, the compound is

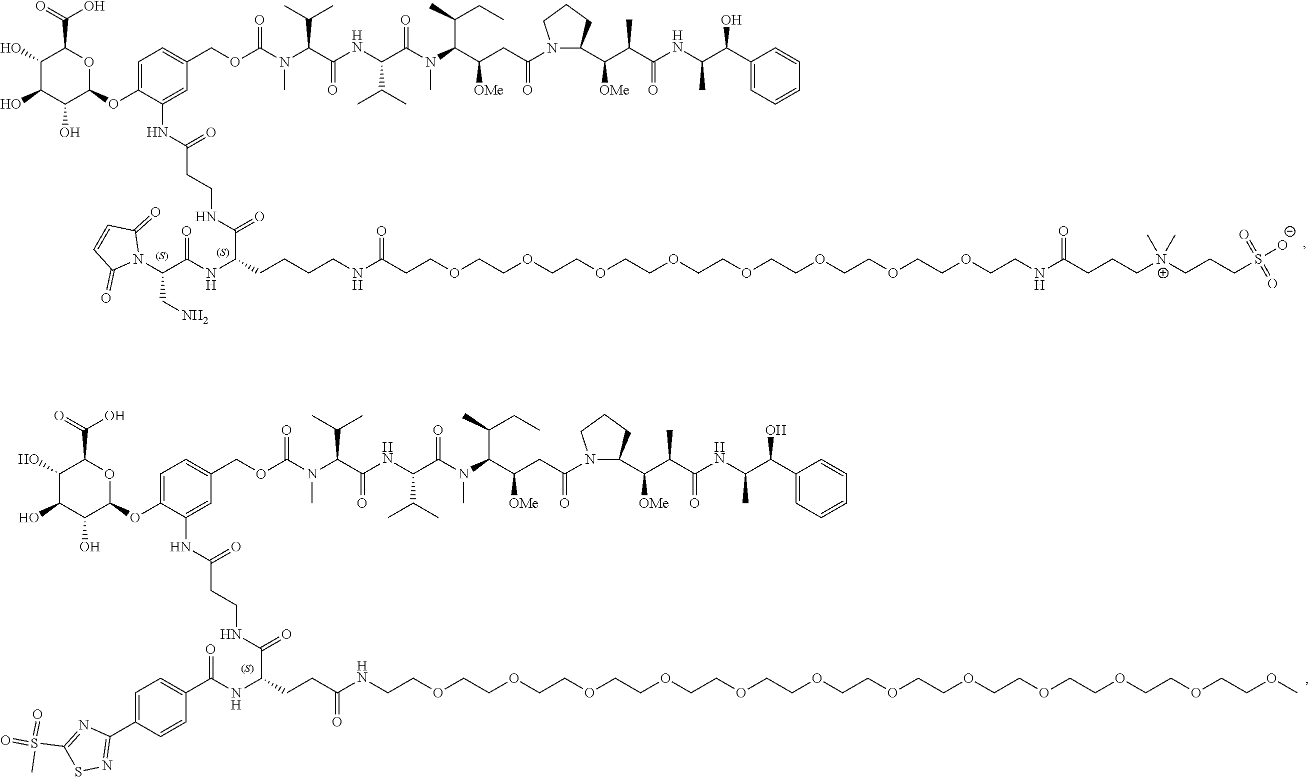

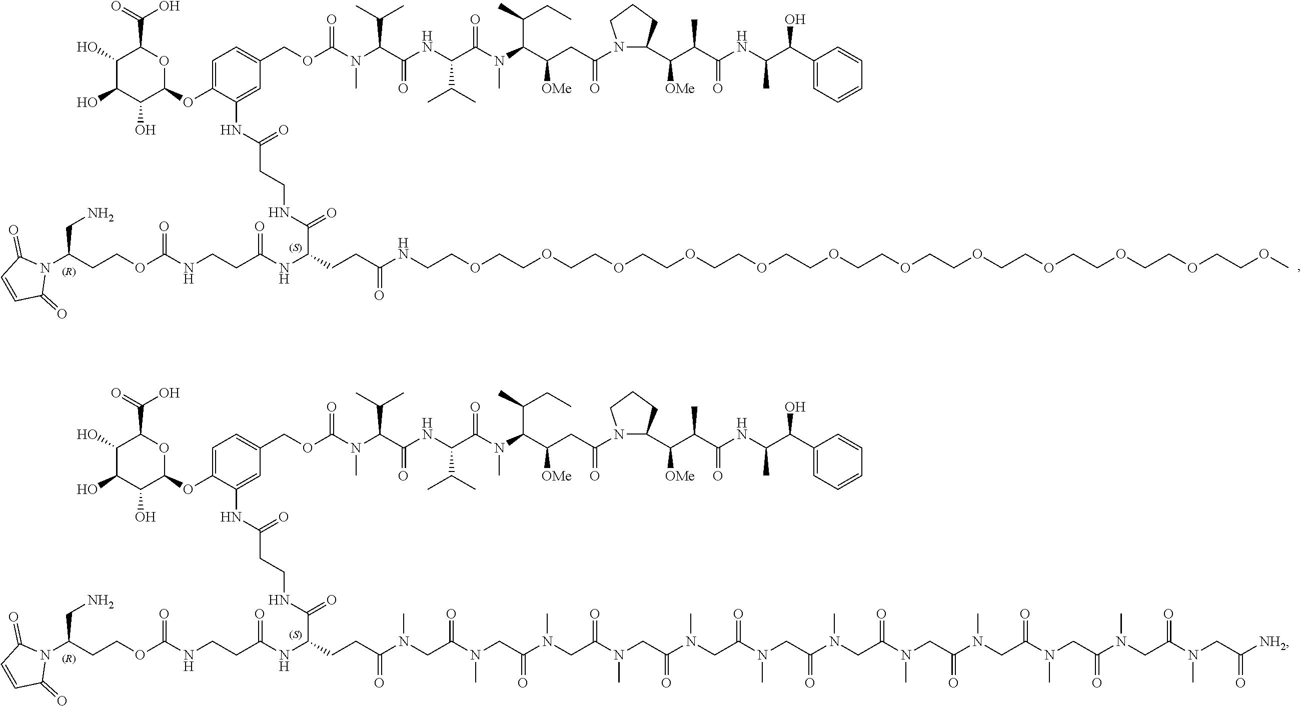

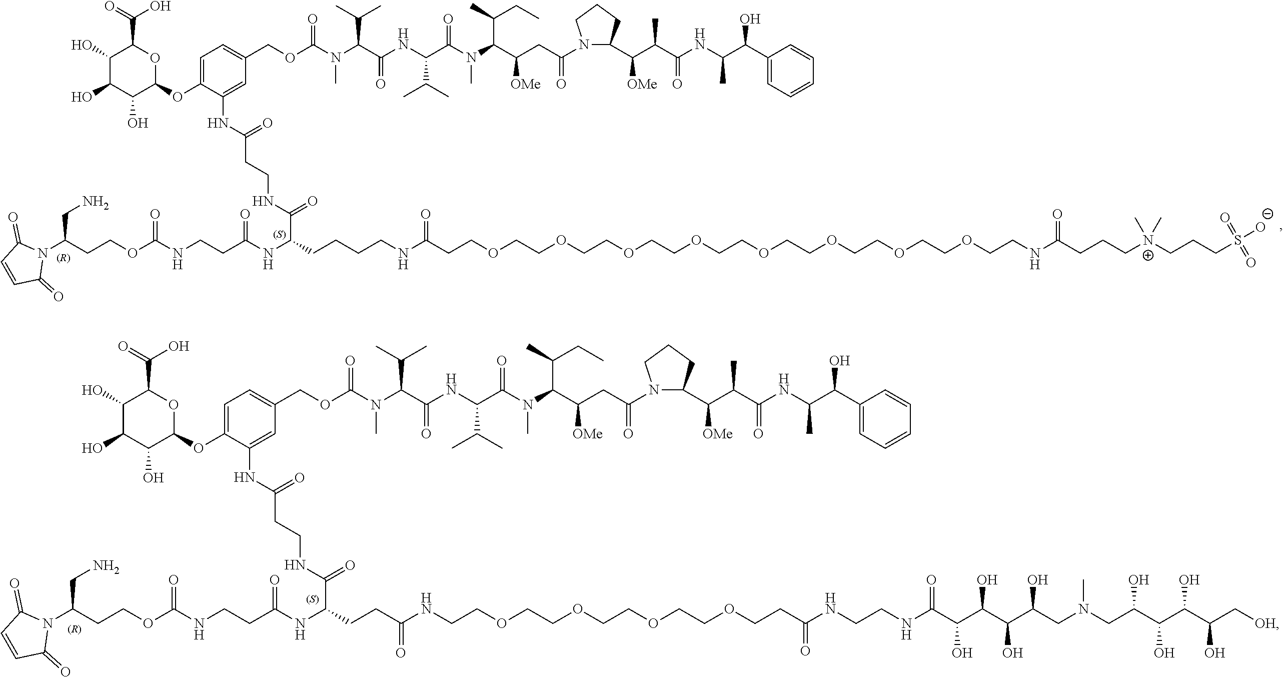

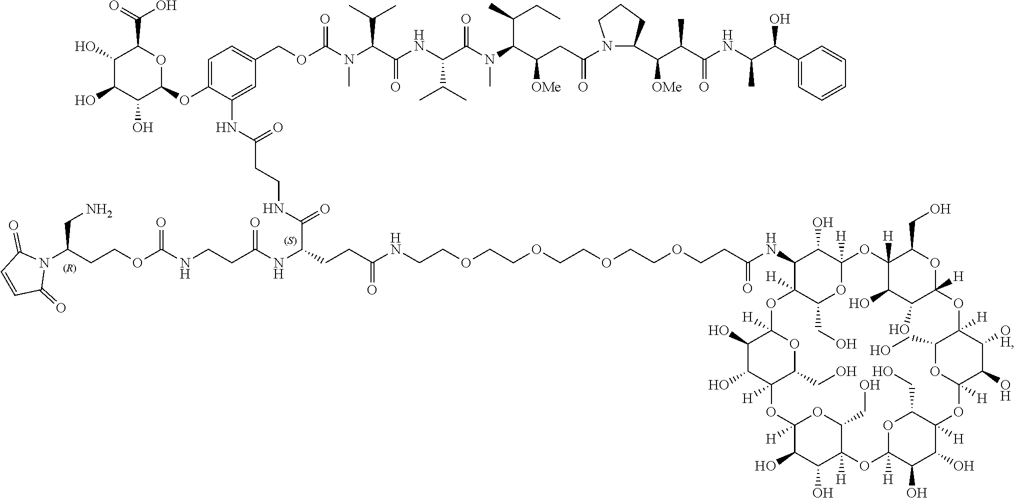

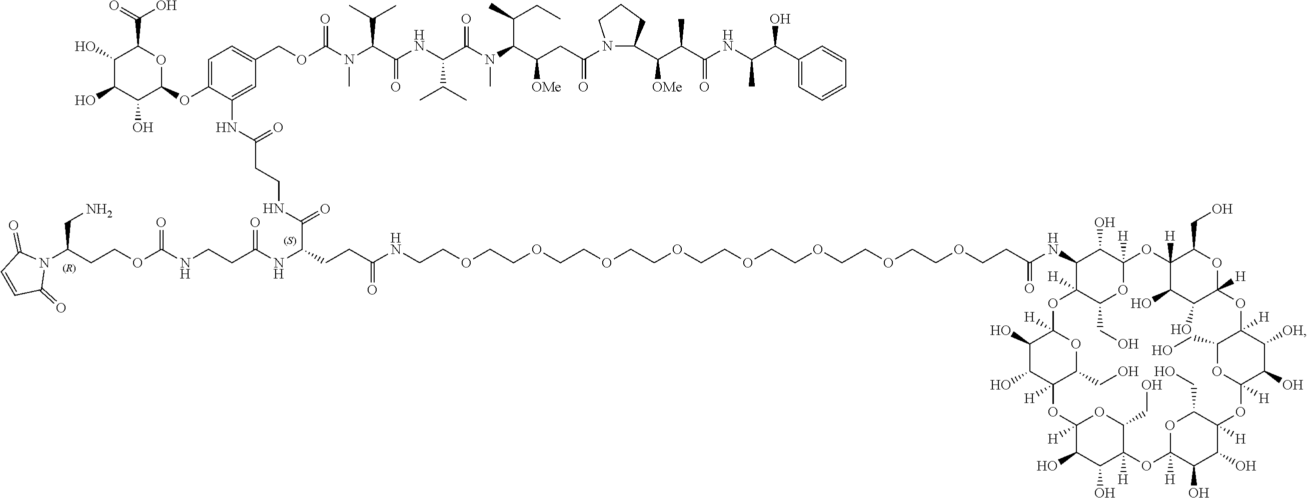

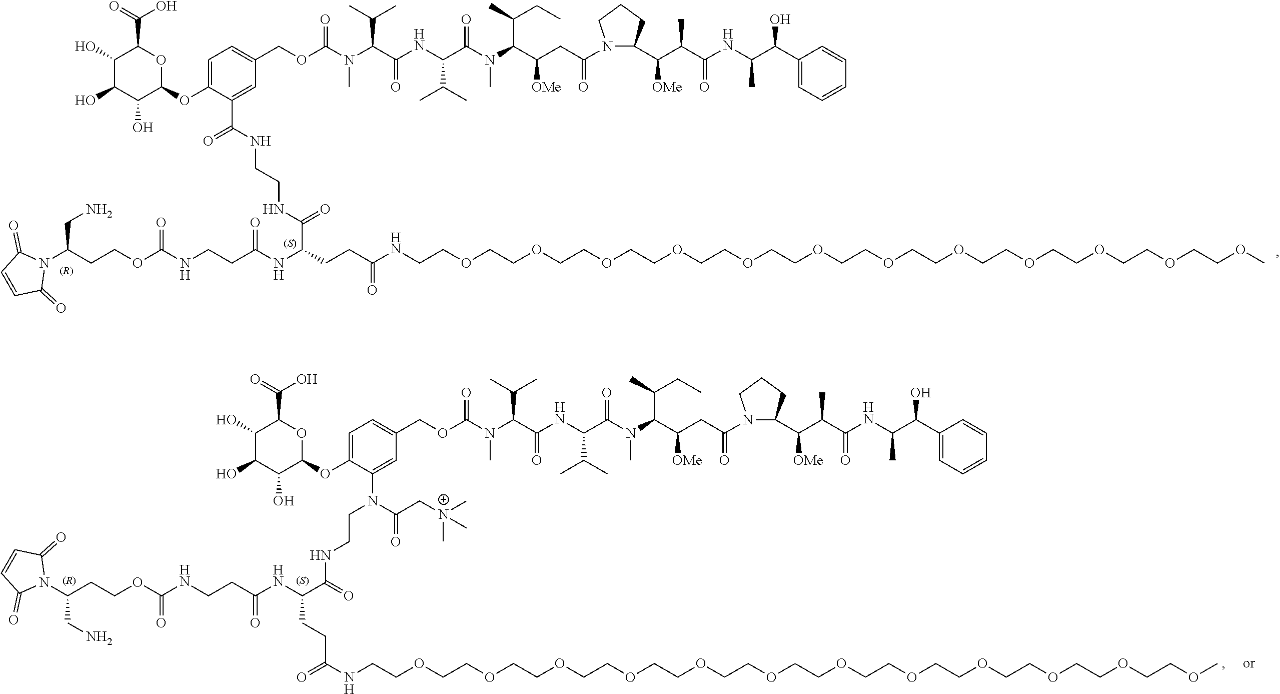

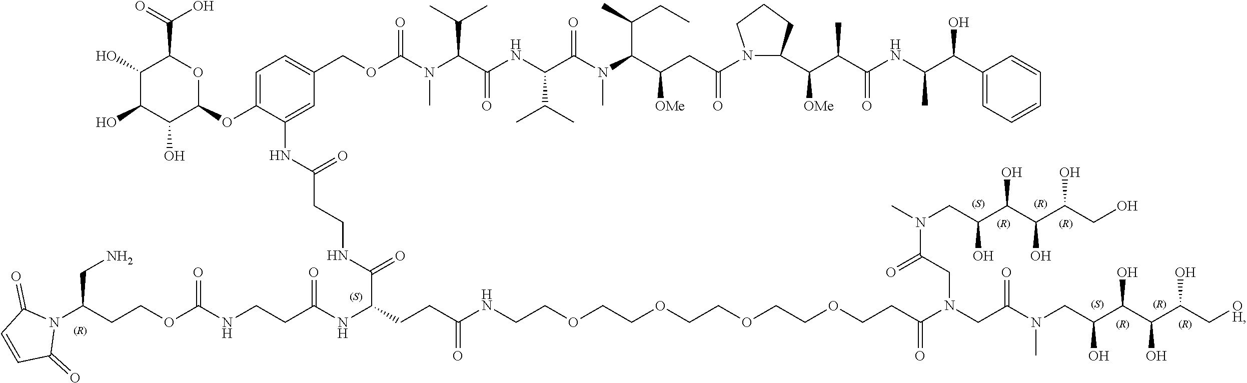

or a pharmaceutically acceptable salt, tautomer, solvate, or stereoisomer thereof.

### Methods of Making the Conjugates

Provided herein are methods of preparing a conjugate by contacting a binding agent (BA) with a conjugator-linker-payload compound under conditions suitable for forming a bond between the binding agent and the conjugator-linker-payload compound. The reaction conditions may be any suitable reaction conditions known in the art. The binding agent may be an antibody and the bond may form an antibody-drug conjugate.

Examples of such reactions are provided in the Examples below.

In some embodiments, methods of making a conjugate including treating or contacting a compound with a binding agent under coupling conditions. The compound may include a reactive linker bonded to at least one payload. The compound may be any of the linker or platform compounds disclosed herein.

### Pharmaceutical Compositions

Also provided herein are compositions, including pharmaceutical compositions, comprising an ADC set forth herein. In some embodiments, the compositions (e.g., pharmaceutical compositions) further comprise a pharmaceutically acceptable excipient.

Pharmaceutical compositions in accordance with the present disclosure can be prepared by mixing an antibody drug conjugate having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to, buffers such as phosphate, citrate, and other organic acids; antioxidants including ascorbic acid and methionine; preservatives (such as octadecyl dimethyl benzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Nos. U.S. Pat. No. 7,871,607 and 2006/0104968. In one aspect, a sHASEGP is combined with one or more additional glycosaminoglycanases such as chondroitinases.

### Methods of Using the Conjugates

In some embodiments, set forth herein is a method of treating a disease or disorder (e.g., a cancer) in a subject (e.g., patient) in need thereof, comprising administering to the patient a therapeutically effective amount of a conjugate disclosed herein.

The conjugates disclosed herein can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injection, such as intravenous or subcutaneous injection, depending in part on whether the administration is brief or chronic. Various dosing schedules, including but not limited to, single or multiple administrations over various time-points, bolus administration, and pulse infusion are contemplated herein.

Conjugates of the disclosure can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

### EXAMPLES

The examples below are intended to be exemplary and should not be considered limiting in any way. Unless otherwise specified, the experimental methods in the Examples described below are conventional methods. Unless otherwise specified, the reagents and materials are commercially available. All solvents and chemicals employed were of analytical grade or chemical purity. Solvents were redistilled before use. Anhydrous solvents were prepared according to standard methods or reference methods. Silica gel (100-200 meshes) for column chromatography and silica gel (GF254) for thin-layer chromatography (TLC) are commercially available from Tsingdao Haiyang Chemical Co., Ltd. or Yantai Chemical Co., Ltd. of China; all were eluted with petroleum ether (60-90° C.)/ethyl acetate (v/v) and visualized by iodine or the solution of molybdphosphoric acid in ethanol unless otherwise specified. All extraction solvents, unless otherwise specified, were dried over anhydrous Na₂SO₄. ¹H NMR spectra were recorded on Bruck-400, Varian 400MR nuclear magnetic resonance spectrometer with TMS (tetramethylsilane) as the internal standard. Coupling constants were given in hertz. Peaks were reported as singlet(s), doublet (d), triplet (t), quartet (q), quintet (p), sextet (h), septet (hept), multiplet (m), or a combination thereof; br stands for broad. LC/MS data was recorded by using Agilent1100,1200 High Performance Liquid Chromatography-Ion Trap Mass Spectrometer (LC-MSD Trap) equipped with a diode array detector (DAD) detected at 214 nm and 254 nm, and an ion trap (ESI source). All compound names except the reagents were generated by ChemDraw® 18.0.

For the sake of conciseness, certain abbreviations are used herein. One example is the single letter abbreviation to represent an amino acid. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

In the following examples, the following abbreviations are used:

| | |
|---|---|
| AcOH | Acetic acid |
| DCM | Dichloromethane |
| DIPEA/DIEA | N,N-Diisopropylethylamine |
| DMF | N,N-Dimethylformamide |
| EDCI | 1-(3-Dimethylaminopropyl)-3-ethylcarbodiimide |
| | hydrochloride |
| EEDQ | 2-Ethoxy-1-ethoxycarbonyl-1,2-dihydroquinoline |
| EtOAc | Ethyl acetate |
| EtOH | Ethanol |
| FA | Formic Acid |
| HATU | O-(7-azabenzotriazol-1-yl)-N,N,N′,N′- |
| | tetramethyluronium hexafluorophosphate |
| HOBt | 1-Hydroxybenzotriazole |
| IPA | Isopropyl alcohol |
| MeCN/ACN | Acetonitrile |
| MeOH | Methanol |
| MMAE | Monomethyl auristatin E |
| Prep-HPLC | Preparative high performance liquid chromatography |
| TEA | Triethyl amine |
| TFA | Trifluoroacetic acid |
| THF | Tetrahydrofuran |
| TsOH•H₂O | p-Toluenesulfonic acid monohydrate |

### UPLC Analysis Methods

Method A: Mobile phase A: 0.1% FA in water, B: MeCN; Gradient: 10% B maintain 0.2 min, 10%-95% B, 5.8 min, 95% B maintain 0.5 min; Flow rate: 0.6 mL/min; Column: ACQUITY UPLC® BEH C18 1.7 μm.
Method B: Mobile phase A: 0.1% FA in water, B: MeCN; Gradient: 10% B maintain 0.5 min, 10%-90% B, 2.5 min, 90% B maintain 0.2 min; Flow rate: 0.6 mL/min; Column: ACQUITY UPLC® BEH C18 1.7 μm.
Method C: Mobile phase A: 0.1% FA in water, B: MeCN; Gradient: 10% B maintain 0.2 min, 10%-90% B, 1.3 min, 90% B maintain 0.3 min; Flow rate: 0.6 mL/min; Column: ACQUITY UPLC® BEH C18 1.7 μm.

### Example Int-1

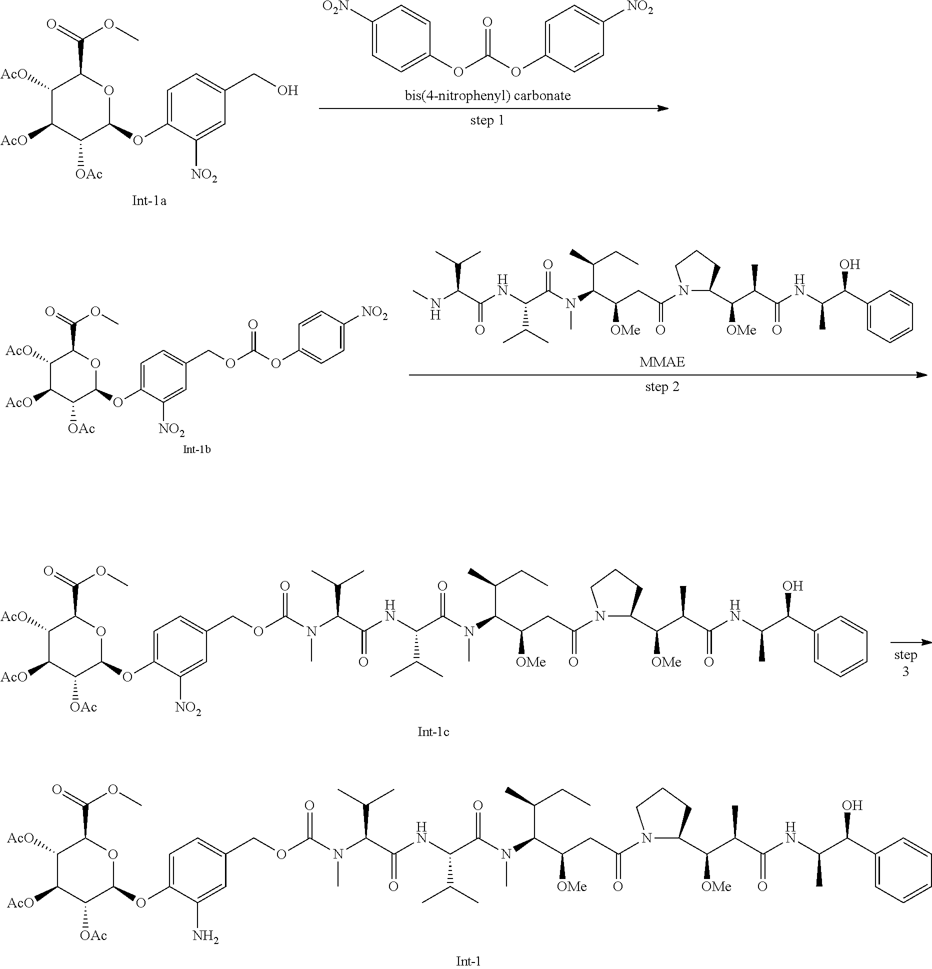

### Step 1: (2S,3S,4S,5R,6S)-2-(methoxycarbonyl)-6-(2-nitro-4-((((4-nitrophenoxy)carbonyl)oxy)methyl)phenoxy)tetrahydro-2H-pyran-3,4,5-triyl triacetate (Int-1b)

To the solution of Int-1a (3.00 g, 6.18 mmol) (commercially available) in dry DMF (60 mL) were added Bis(4-nitrophenyl) carbonate (2.85 g, 9.271 mmol) and DIPEA (2.18 mL, 12.36 mmol) at 0° C., stirred at r.t. for 3 h. The reaction was quenched with aq. AcOH, extracted with EtOAc (100 mL*3), washed with water (100 mL) and brine (100 mL). The organic phase was concentrated under reduced pressure. The residue was purified by silica gel column chromatography (EtOAc/Petroleum ether=30/70) to give Int-1b (4 g, 99.5% yield) as an off-white solid. MS (ESI) m/z: 673.2 [M+Na]⁺.

### Step 2: (2S,3R,4S,5S,6S)-2-(4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-nitrophenoxy)-6-(methoxycarbonyl)tetrahydro-2H-pyran-3,4,5-triyl triacetate (Int-1c)

To the solution of Int-1b (2.00 g, 3.08 mmol) and MMAE (2.45 g, 3.38 mmol) in dry DMF (30 mL) were added HOBt (126 mg, 0.92 mmol) and DIPEA (1.66 mL, 9.22 mmol), stirred at r.t. for 16 h. The organic phase was concentrated and purified by silica gel column chromatography (CH₂Cl₂/MeOH=90/10) to give Int-1c (3.50 g, 92.6% yield) as a white solid. MS (ESI) m/z: 1229.7 [M+H]⁺.

### Step 3: (2S,3R,4S,5S,6S)-2-(2-amino-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-6-(methoxycarbonyl)tetrahydro-2H-pyran-3,4,5-triyl triacetate (Int-1)

To the solution of Zn powder (6.96 g, 105.34 mmol) in EtOH (10.0 mL) and water (1 mL) was added AcOH (0.99 mL, 17.08 mmol) at 0° C., stirred at 0° C. for 1 h. Then to the above mixture was added Int-1c (3.50 g, 2.85 mmol) in EtOH (10 mL) slowly at 0° C., stirred at r.t. for 4 h. The resulting solution was filtrated and washed with EtOH (100 mL). The filtrate was concentrated to remove EtOH. To the residue was added water (100 mL) and extracted with EtOAc (100 mL*3). The combined organic layers were dried over Na₂SO₄ and concentrated. The residue was purified by silica gel column chromatography (CH₂Cl₂/MeOH=90/10) to give Int-1 (3.30 g, 96.6% yield) as a white solid. MS (ESI) m/z: 1199.8 [M+H]⁺.

### Example Int-2

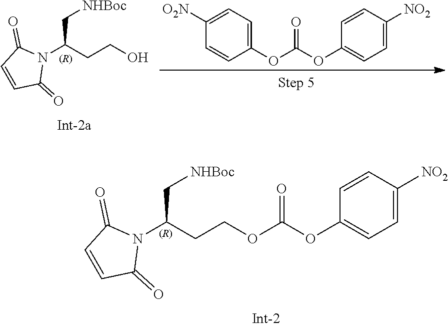

Int-2 was synthesized according to a modified synthetic procedure of reference (Tetrahedron Letters 54 (2013) 349-3495).

### Example Int-3

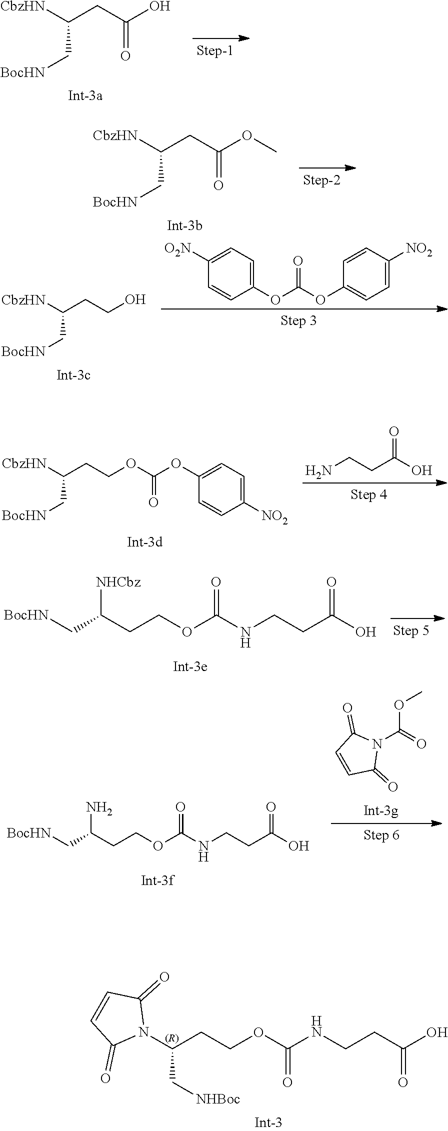

### Step 1: Methyl (R)-3-(((benzyloxy)carbonyl)amino)-4-((tert-butoxycarbonyl)amino)butanoate (Int-3b)

Int-3a (2.00 g, 5.68 mmol) and K₂CO₃ (863 mg, 6.24 mmol) were added into DMF (10 mL) followed by the dropwise addition of CH₃I (1.61 g, 11.35 mmol) at 0° C. The resulting mixture was stirred at 0° C. for 20 min and allowed to warm to 25° C. and further stirred at 25° C. for 60 min. The reaction process was monitored by TLC (Petroleum ether/EtOAc) and LCMS. After complete reaction, the reaction mixture was diluted with EtOAc (80 mL) and washed with brine (30 mL*3) and H₂O (30 mL*2). The organic layer was dried over anhydrous Na₂SO₄, filtered, and concentrated under reduced pressure to afford the methyl ester of Int-3b (2.08 g, quant.) as a light-yellow solid. MS (ESI) m/z: 267.2 [M-Boc+H]⁺.

### Step 2: Benzyl tert-butyl (4-hydroxybutane-1,2-diyl)(R)-dicarbamate (Int-3c)

Int-3b (1.00 g, 2.73 mmol) was dissolved in MeOH (15 mL) followed by the addition of LiBH₄ (2 M stock solution in THF, 6.80 mL) at 0° C. The resulting mixture was stirred at 25° C. for 2 h. Reaction process was monitored by LCMS and TLC. After complete reaction, saturated aqueous NH₄Cl (10 mL) was added to quench the reaction. The reaction mixture was diluted with H₂O (80 mL) and extracted with EtOAc (50 mL*3). The combined organic layers were washed with brine (40 mL*2) and water (40 mL*2), dried over anhydrous Na₂SO₄, filtered, and concentrated under reduced pressure and further purified by flash column chromatography (Petroleum ether/EtOAc) to afford Int-3c (760 mg, 82.3% yield) as a white solid. MS (ESI) m/z: 239.2 [M-Boc+H]⁺.

### Step 3: Benzyl tert-butyl (4-(((4-nitrophenoxy)carbonyl)oxy)butane-1,2-diyl)(R)-dicarbamate (Int-3d)

Int-3c (300 mg, 0.89 mmol) and Bis(4-nitrophenyl) carbonate (405 mg, 1.33 mmol) were dissolved in DMF (5 mL) followed by the addition of DIEA (229 mg, 1.77 mmol). The resulting mixture was stirred at 25° C. for 1.5 h. After complete reaction. The reaction mixture was diluted with EtOAc (100 mL) and washed with brine (35 mL*2) and water (35 mL*2). The organic layer was dried over anhydrous Na₂SO₄, filtered, and concentrated under reduced pressure to afford Int-3d as a white solid (371 mg, 83.1% yield). MS (ESI) m/z: 404.4 [M-Boc+H]⁺.

### Step 4: (9H-fluoren-9-yl)methyl tert-butyl (4-(((3-(dimethylamino)-3-oxopropyl)carbamoyl)oxy)butane-1,3-diyl) (S)-dicarbamate (Int-3e)

Int-3d (420 mg, 0.83 mmol) and 3-aminopropanoic acid (149 mg, 1.67 mmol) were dissolved in DMF (5 mL) followed by the addition of aqueous NaHCO₃ (1M, 5 mL). The resulting mixture was stirred at 25° C. for 2.5 h. After complete reaction, the reaction mixture was concentrated and purified by flash column chromatography (DCM/MeOH) to afford Int-3e as a pale-yellow solid (365 mg, 96.5% yield). MS (ESI) m/z: 354.4 [M-Boc+H]⁺.

### Step 5: (R)-7-(2,5-dioxo-2,5-dihydro-1H-pyrrol-1-yl)-2,2-dimethyl-4,11-dioxo-3,10-dioxa-5,12-diazapentadecan-15-oic acid (Int-3f)

Int-3e (360 mg, 0.79 mmol) was dissolved in MeOH (18 mL) followed by the addition of Pd/C (wet base, 108 mg). The resulting mixture was stirred at r.t. under H₂ (15 psi) for 2 h. After complete reaction, the reaction mixture was filtered and concentrated under reduced pressure to afford Int-3f as a clear syrup (252 mg, 99.4% yield). The crude product was used directly in next step without purification. MS (ESI) m/z: 320.3 [M+H]⁺.

### Step 6: (R)-7-(2,5-dioxo-2,5-dihydro-1H-pyrrol-1-yl)-2,2-dimethyl-4,11-dioxo-3,10-dioxa-5,12-diazapentadecan-15-oic acid (Int-3)

Int-3f (250 mg, 0.78 mmol) and Int-3g (243 mg, 1.57 mmol) were dissolved in a mixed solvent of ACN (8 mL) and aqueous NaHCO₃ (1M, 16 mL). The resulting mixture was stirred at 0° C. for 1 h and further stirred at 25° C. until the reaction was complete. Then the reaction mixture was acidified with aq. KHSO₄ (20 mL) and extracted with EtOAc (35 mL*3). The combined organic layers were dried over anhydrous Na₂SO₄, filtered, and concentrated under reduced pressure to afford a yellow oil as the crude product which was purified by flash column chromatography to afford Int-3 (280 mg, 89.6% yield) as a white solid. MS (ESI) m/z: 422.3 [M+Na]⁺.

¹H NMR (400 MHz, d₆-DMSO) δ 12.48 (s, 1H), 7.03-7.01 (m, 2H), 6.99 (s, 2H), 4.08-4.03 (m, 3H), 3.86-3.83 (m, 2H), 3.14-3.11 (m, 2H), 2.35 (t, J=7.2 Hz, 2H), 2.16-2.09 (m, 1H), 1.91-1.84 (m, 1H), 1.32 (s, 9H).

### Example Int-4

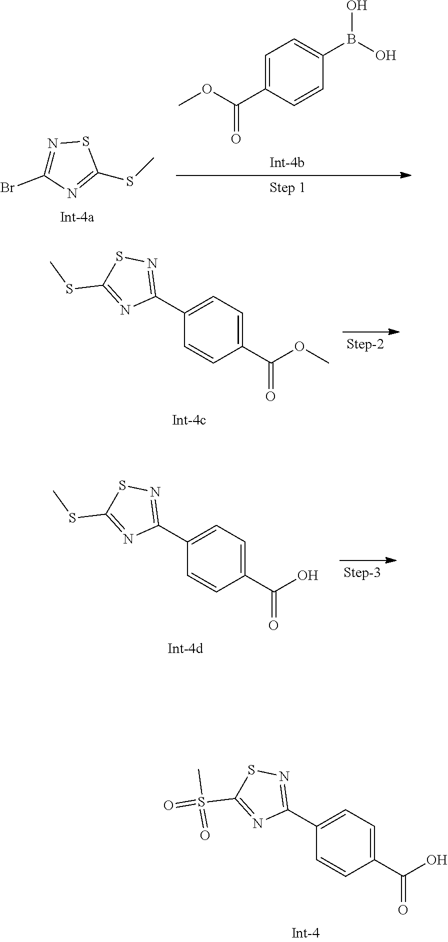

### Step 1: methyl 4-(5-(methylthio)-1,2,4-thiadiazol-3-yl)benzoate (Int-4c)

To a solution of compound Int-4a (100 mg, 0.47 mmol) in toluene (4 mL) and H₂O (1 mL) were added compound Int-4b (109.72 mg, 0.568 mmol), K₂CO₃ (168 mg, 0.947 mmol) and Pd(dppf)Cl₂·DCM (34.6 mg, 0.047 mmol). The mixture was stirred at 110° C. for 3 h under N₂ atmosphere. The mixture was filtered through a pad of celite, diluted with EA (100 mL), washed by brine (50 mL+4). The organic layer was dried over anhydrous Na₂SO₄, filtered and concentrated. The crude was purified by flash column chromatography (eluted with PE:EA=0-40%). Compound Int-4c (56 mg, 44.4% yield) was obtained as off-white solid. MS (ESI) m/z: 267.1 [M+H]⁺.

### Step 2:4-(5-(methylthio)-1,2,4-thiadiazol-3-yl)benzoic acid (Int-4d)

To a solution of compound Int-4c (54 mg, 0.20 mmol) in MeOH (3 mL) and H₂O (1 mL) was added LiOH (17 mg, 0.41 mmol). The mixture was stirred at r.t. for 2 h. The mixture was adjusted to pH 7 and purified by prep-HPLC (FA condition) to give compound Int-4d (36 mg, 70.3% yield) as white solid. MS (ESI) m/z: 253.1 [M+H]⁺.

### Step 3:4-(5-(methylsulfonyl)-1,2,4-thiadiazol-3-yl)benzoic acid (Int-4)

To a solution of compound Int-4d (35 mg, 0.14 mmol) in DCM (3 mL) and THF (3 mL) was added m-CPBA (96 mg, 0.55 mmol). The mixture was stirred at room temperature for 16 h. The mixture was concentrated and purified by prep-HPLC (Method: Column: XBridge Prep C18 OBD 5 um 19*250 mm; Mobile phase: A-water (0.1% TFA): B-acetonitrile; Flow rate: 20 mL/min). Compound Int-4 (12 mg, 99% purity) was obtained as white solid. MS (ESI) m/z: 284.8 [M+H]⁺.

### Example 1: Synthesis of Conjugator-Linker-Payloads

### Example 1-1

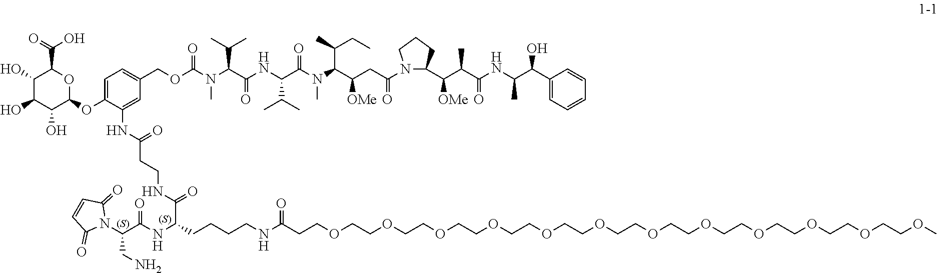

1-1 was synthesized according to PCT publication WO2017165851.

### Example 1-2

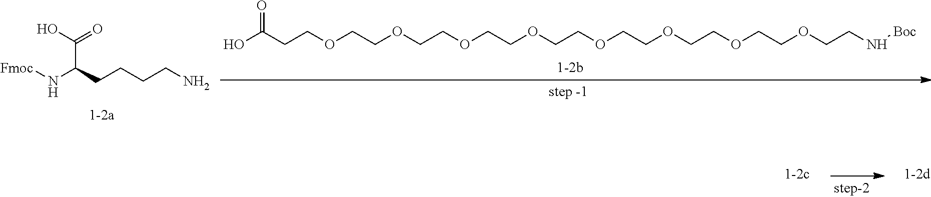

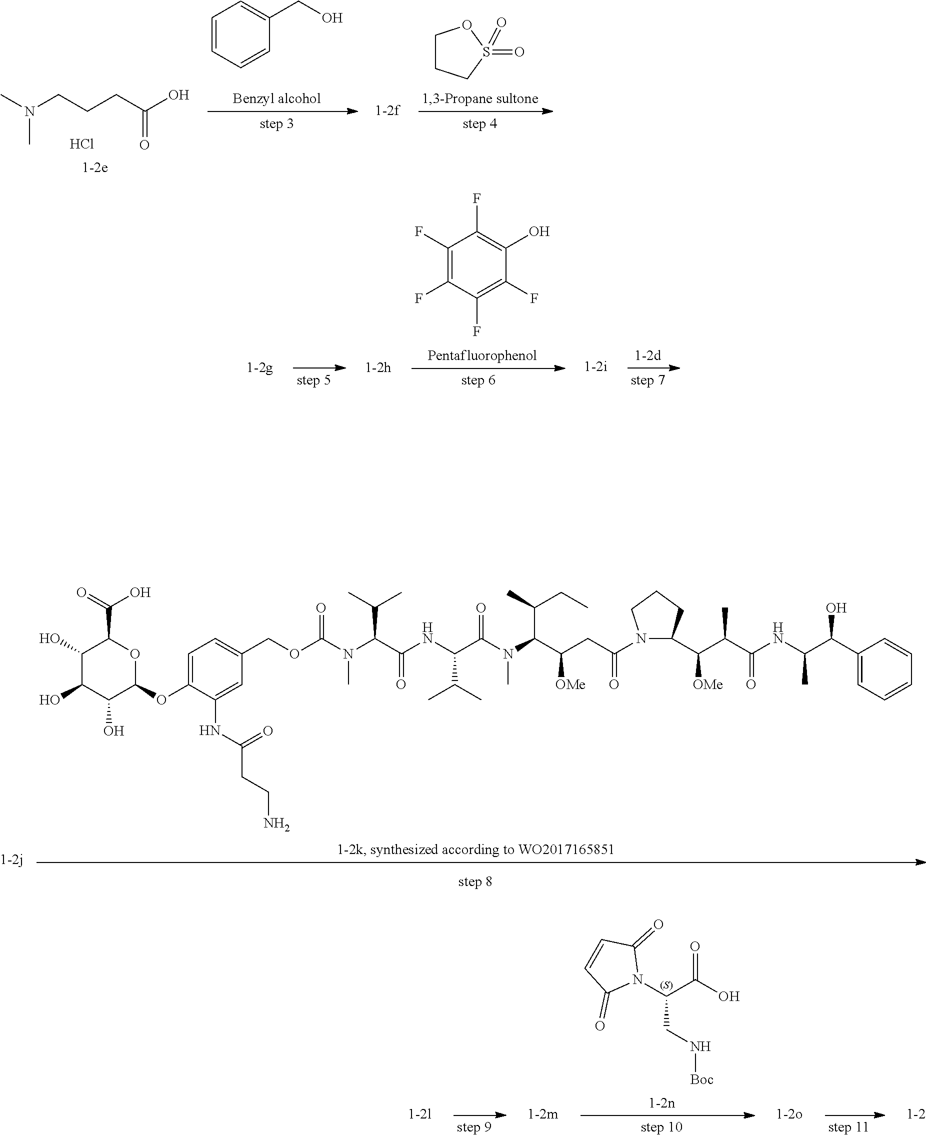

### Step 1: (R)-38-((((9H-fluoren-9-yl)methoxy)carbonyl)amino)-2,2-dimethyl-4,32-dioxo-3,8,11,14,17,20,23,26,29-nonaoxa-5,33-diazanonatriacontan-39-oic acid (1-2c)

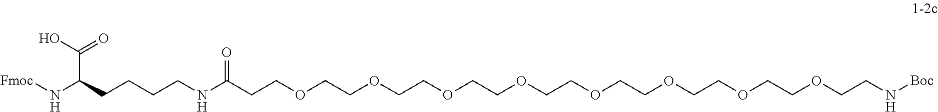

To the solution of 1-2b (350 mg, 0.19 mmol) (commercially available) in dry DMF (5 mL) were added HATU (236 mg, 0.61 mmol) and DIPEA (0.23 mL, 1.29 mmol), stirred at r.t. for 20 min. The to the above mixture was added 1-2a (264.5 mg, 0.71 mmol) (commercially available), stirred at r.t. for 10 min. The resulting solution was purified by prep-HPLC (Method: column XBridge Prep C18 OBD 5 μm 19*150 mm; Mobile phase: A-water (0.1% FA): B-acetonitrile; Flow rate: 20 mL/min, the fraction was lyophilized to give 1-2c (345 mg, 59.9% yield) as a colorless oil. MS (ESI) m/z: 892.8 [M+H]⁺.

### Step 2: (R)-33-((((9H-fluoren-9-yl)methoxy)carbonyl)amino)-1-amino-27-oxo-3,6,9,12,15,18,21,24-octaoxa-28-azatetratriacontan-34-oic acid (1-2d) trifluoroacetate

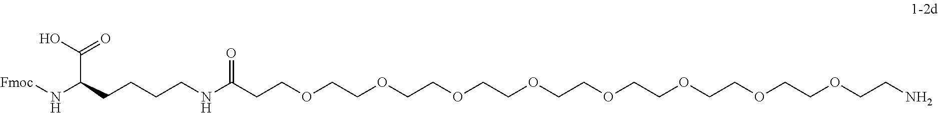

To the solution of 1-2c (180.0 mg, 0.20 mmol) in CH₂Cl₂ (6 mL) was added TFA (1.56 mL, 20.18 mmol) at 0° C., stirred at 0° C. for 30 min. The resulting solution was purified by prep-HPLC (Method: column XBridge Prep C18 OBD 5 μm 19*150 mm; Mobile phase: A-water (0.1% TFA): B-acetonitrile; Flow rate: 20 mL/min, the fraction was lyophilized to give 1-2d (trifluoroacetate) (150 mg, 82.1% yield) as a white solid. MS (ESI) m/z: 792.8 [M+H]⁺.

### Step 3: benzyl 4-(dimethylamino)butanoate (1-2f)

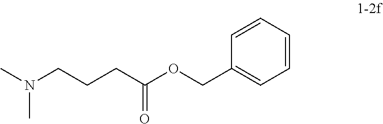 [SMILES: CN(C)CCCC(=O)OCc1ccccc1]

To the solution of 1-2e (1.0 g, 5.97 mol) and Benzyl alcohol (1.11 g, 10.14 mmol) in toluene (8 mL) as added TsOH·H₂O (1.20 g, 6.26 mmol). The mixture was degassed for 10 min and refilled with N₂, and the toluene (10 mL) was heated to reflux to remove water by Dean-Stark trap, stirred at 130° C. for 4 h. After cooling the reaction mixture, the toluene phase was extracted 4 times with water. The pH of the aqueous phase was adjusted to pH 10 with 1N NaOH. The aqueous phase was then extracted with EtOAc (50 mL*3). The organic layer was evaporated and purified by silica gel column chromatography (CH₂Cl₂/MeOH=90/10) to give 1-2f (910 mg, 68.9% yield) as a yellow oil. MS (ESI) m/z: 223.2 [M+H]⁺.

### Step 4: 3-((4-(benzyloxy)-4-oxobutyl)dimethylammonio) propane-1-sulfonate (1-2g)

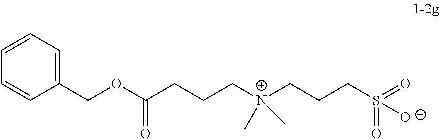 [SMILES: C[N+](C)(CCCC(=O)OCc1ccccc1)CCCS(=O)(=O)[O-]]

To the solution of 1-2f (910 mg, 4.11 mol) and 1,3-Propane sultone (761 mg, 6.17 mmol) in dry DMF (10 mL) was added 2,6-Di-Tert-Butylpyridine (1.21 g, 6.17 mmol). The mixture was stirred at 150° C. for 5 h. After cooling, the reaction mixture was concentrated. Partition the residue between EtOAc (50 mL) and water (50 mL). The aqueous phase was then extracted 3 times with EtOAc and the pH of the solution was adjusted to 10 with NH₃H₂O and further extract with EtOAc (50 mL*2). The aqueous solution was concentrated under reduced pressure and purified by prep-HPLC (Method: column XBridge Prep C18 OBD 5 μm 19*150 mm; Mobile phase: A-water (0.1% FA): B-acetonitrile; Flow rate: 20 mL/min, the fraction was lyophilized to give 1-2g (510 mg, 36.1% yield) as a white solid. MS (ESI) m/z: 344.4 [M+H]⁺

### Step 5: 3-((3-carboxypropyl)dimethylammonio) propane-1-sulfonate (1-2h)

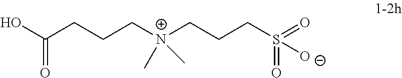 [SMILES: C[N+](C)(CCCC(=O)O)CCCS(=O)(=O)[O-]]

Treat a solution of 1-2g (510 mg, 1.49 mmol) in MeOH (10 mL) with 100 mg of 10% Pd/C. Hydrogenate the suspension in a parr shaker at 40 psi for 2 hours at room temperature. Filter the reaction mixture. The methanol solution was concentrated under reduced pressure to give 1-2h (375 mg, crude) as a colorless oil, which was directly used in the next step. MS (ESI) m/z: 254.2 [M+H]⁺.

### Step 6: 3-(dimethyl (4-oxo-4-(perfluorophenoxy)butyl) ammonio) propane-1-sulfonate (1-2i)

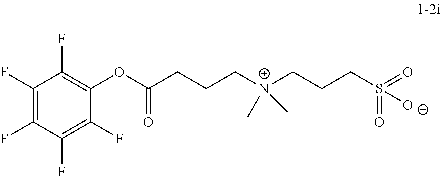 [SMILES: C[N+](C)(CCCC(=O)Oc1c(F)c(F)c(F)c(F)c1F)CCCS(=O)(=O)[O-]]

To the solution of 1-2h (260 mg, 1.03 mmol) in 0.1N HCl (2 mL) and MeCN (2 mL) were added Pentafluorophenol (382 mg, 2.05 mmol) and EDCI (497 mg, 2.57 mmol), stirred at r.t. for 16 h. The resulting solution was purified by prep-HPLC (Method: column XBridge Prep C18 OBD 5 um 19*150 mm; Mobile phase: A-water (0.1% TFA): B-acetonitrile: Flow rate: 20 mL/min. the fraction was lyophilized to give 1-2i (260 mg, 60.4% yield) as a colorless oil. MS (ESI) m/z: 420.4 [M+H]⁺.

### Step 7: (R)-5-carboxy-1-(9H-fluoren-9-yl)-43,43-dimethyl-3,11,39-trioxo-2,14,17,20,23,24,26,29,32,35-decaoxa-4,10,38,43-tetraazahexatetracontan-43-ium-46-sulfonate (1-2j)

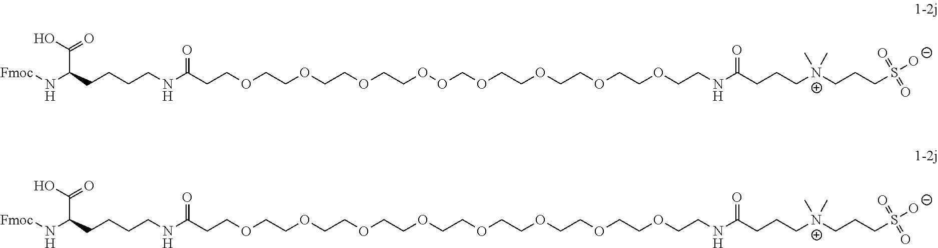

To the solution of 1-2i (75 mg, 0.18 mmol) and 1-2d (72 mg, 0.09 mmol) in water (1 mL) and MeCN (1 mL) was added sat. NaHCO₃ (0.5 mL), stirred at r.t. for 1 h. The resulting solution was purified prep-HPLC (Method: column XBridge Prep C18 OBD 5 um 19*150 mm; Mobile phase: A-water (0.1% TFA): B-acetonitrile; Flow rate: 20 mL/min, the fraction was lyophilized to give 1-2j (75 mg, 40.8% yield) as a white solid. MS (ESI) m/z: 1029.9 [M+H]⁺.

### Step 8: (S)-5-((3-((5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-(((2S,3R,4S,5S,6S)-6-carboxy-3,4,5-trihydroxytetrahydro-2H-pyran-2-yl)oxy)phenyl)amino)-3-oxopropyl)carbamoyl)-1-(9H-fluoren-9-yl)-43,43-dimethyl-3,11,39-trioxo-2,14,17,20,23,26,29,32,35-nonaoxa-4,10,38,43-tetraazahexatetracontan-43-ium-46-sulfonate (1-2l)

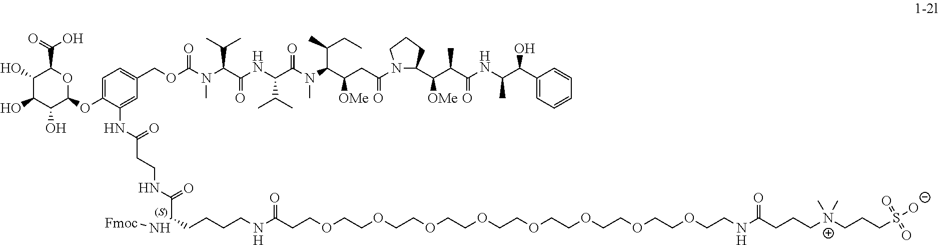

To the solution of 1-2j (73 mg, 0.07 mmol) in dry DMF (2 mL) were added HATU (33 mg, 0.085 mmol) and DIPEA (0.025 mL, 0.14 mmol), stirred at r.t. for 10 min. To the above mixture was added 1-2k (89.3, 0.078 mmol) (synthesized according to PCT publication WO2017165851), stirred at r.t. for 10 min. The resulting solution was purified by prep-HPLC (Method: column XBridge Prep C18 OBD 5 um 19*150 mm; Mobile phase: A-water (0.1% TFA): B-acetonitrile: Flow rate: 20 mL/min, the fraction was lyophilized to give 1-2l (90 mg, 59.1% yield) as a white solid. MS (ESI) m/z: 2140.5 [M+H]⁺.

### Step 9: (S)-42-amino-47-((5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-(((2S,3R,4S,5S,6S)-6-carboxy-3,4,5-trihydroxytetrahydro-2H-pyran-2-yl)oxy)phenyl)amino)-4,4-dimethyl-8,36,43,47-tetraoxo-12,15,18,21,24,27,30,33-octaoxa-4,9,37,44-tetraazaheptatetracontan-4-ium-1-sulfonate (1-2m)

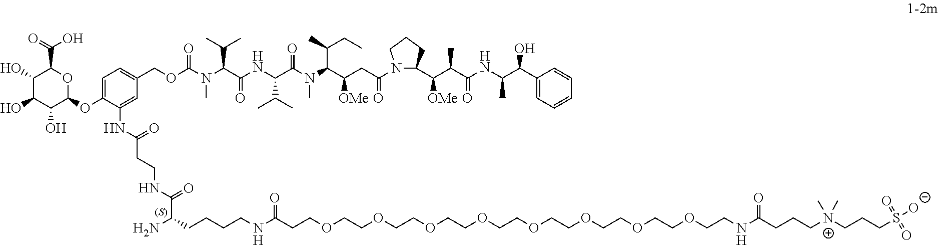

To the solution of 1-2l (90 mg, 0.042 mmol) in dry DMF (2 mL) was added Et₂NH (0.088 mL, 0.84 mmol), stirred at r.t. for 30 min. 1-2m (80 mg, 0.042 mmol, crude) was co-evaporated with toluene five times to remove H₂O. MS (ESI) m/z: 1918.2 [M+H]⁺.

### Step 10: (7S,10S)-10-((3-((5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-(((2S,3R,4S,5S,6S)-6-carboxy-3,4,5-trihydroxytetrahydro-2H-pyran-2-yl)oxy)phenyl)amino)-3-oxopropyl)carbamoyl)-7-(2,5-dioxo-2,5-dihydro-1H-pyrrol-1-yl)-2,2,48,48-tetramethyl-4,8,16,44-tetraoxo-3,19,22,25,28,31,34,37,40-nonaoxa-5,9,15,43,48-pentaazahenpentacontan-48-ium-51-sulfonate (1-2o)

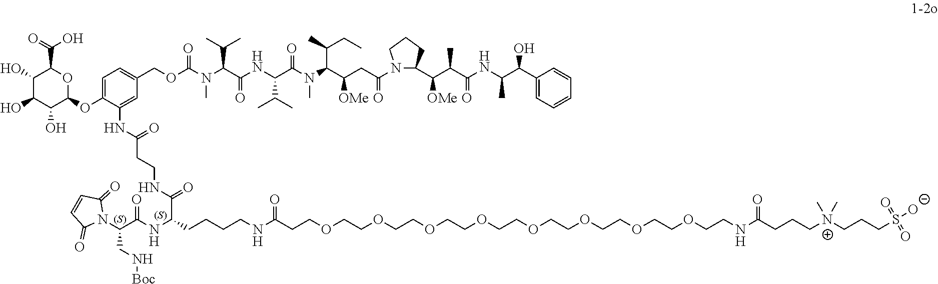

To the solution of 1-2n (15 mg, 0.05 mmol) (synthesized according to PCT publication WO2017165851) in dry DMF (2 mL) were added HATU (26 mg, 0.05 mmol) and DIPEA (0.015 mL, 0.083 mmol), stirred at r.t. for 10 min. Then to the above mixture was added 1-2m (80 mg, 0.042 mmol), stirred at r.t. for 10 min. The resulting solution was purified by prep-HPLC (Method: column XBridge Prep C18 OBD 5 um 19*150 mm; Mobile phase: A-water (0.1% FA): B-acetonitrile; Flow rate: 20 mL/min, the fraction was lyophilized to give 1-20 (30 mg, 32.9% yield) as a white solid. MS (ESI) m/z: 2184.5 [M+H]⁺.

### Step 11: (S)-42-((S)-3-amino-2-(2,5-dioxo-2,5-dihydro-(H-pyrrol-1-yl)propanamido)-47-((5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-(((2S,3R,4S,5S,6S)-6-carboxy-3,4,5-trihydroxytetrahydro-2H-pyran-2-yl)oxy)phenyl)amino)-4,4-dimethyl-8,36,43,47-tetraoxo-12,15,18,21,24,27,30,33-octaoxa-4,9,37,44-tetraazaheptatctracontan-4-ium-1-sulfonate (1-2) trifluoroacetate

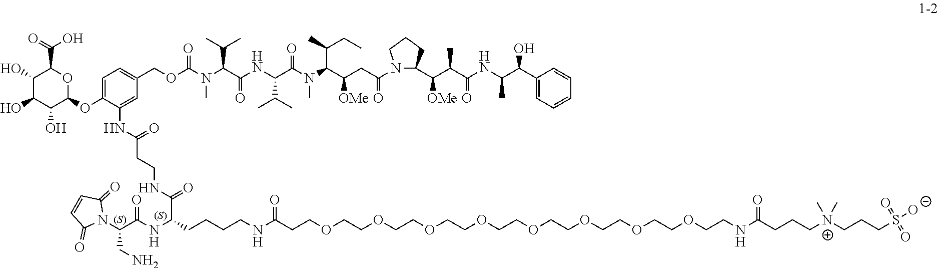

To the solution of 1-20 (30 mg, 0.014 mmol) in CH₂Cl₂ (0.4 mL) was added TFA (0.1 mL, 4.81 mmol) at 0° C., stirred at 0° C. for 30 min. The resulting solution was purified by prep-HPLC (Method: column XBridge Prep C18 OBD 5 μm 19*150 mm; Mobile phase: A-water (0.1% TFA): B-acetonitrile; Flow rate: 20 mL/min, the fraction was lyophilized to give 1-2 (trifluoroacetate) (22.5 mg, 74.5% yield) as a white solid. MS (ESI) m/z: 2084.4 [M+H]⁺.

### Example 1-3

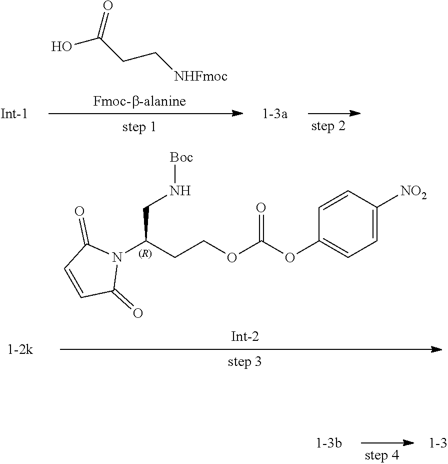

### Step 1: (2S,3R,4S,5S,6S)-2-(2-(3-((((9H-fluoren-9-yl)methoxy)carbonyl)amino)propanamido)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-6-(methoxycarbonyl)tetrahydro-2H-pyran-3,4,5-triyl triacetate (1-3a)

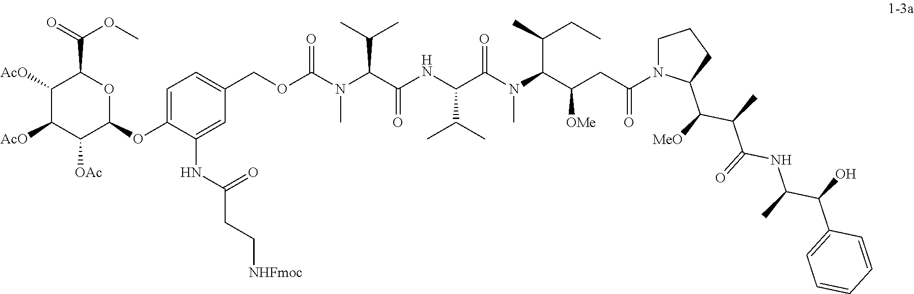

To the solution of Int-1 (400 mg, 0.33 mmol) and Fmoc-β-alanine (126 mg, 0.33 mmol) in dry DMF (3.5 mL) were added HATU (154 mg, 0.40 mmol) and 2,6-lutidine (0.12 mL, 1.00 mmol), stirred at r.t. for 20 h. The resulting solution was quenched with sat. NH₄Cl, extracted with CH₂Cl₂ (50 mL*3). The organic phase was concentrated and purified by silica gel column chromatography (CH₂Cl₂/MeOH=98/2) to give 1-3a (398 mg, 79.9% yield) as a colorless oil. MS (ESI) m/z: 1494.3 [M+H]⁺.

### Step 2: (2S,3S,4S,5R,6S)-6-(2-(3-aminopropanamido)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (1-2k) trifluoroacetate

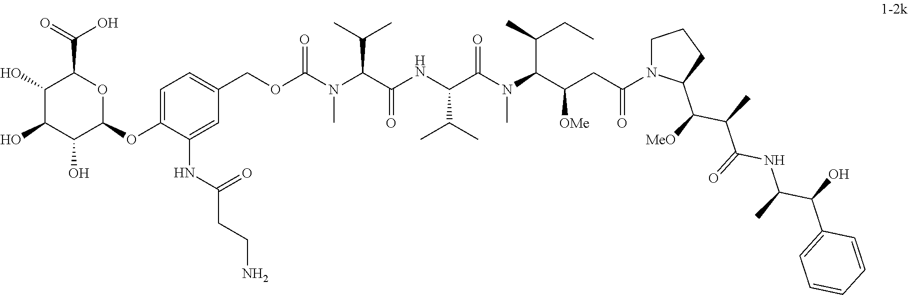 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2O[C@H](C(=O)O)[C@@H](O)[C@H](O)[C@H]2O)c(NC(=O)CCN)c1)C(C)C]

To the solution of 1-3a (398 mg, 0.27 mmol) in water (2.5 mL) and THF (2.5 mL) was added 1N LiOH (2.34 mL, 1.87 mmol) at 0° C., stirred at r.t. for 3 h. The pH was adjusted to around 4 by progressively adding FA. The resulting solution was purified by prep-HPLC (Method: column XBridge Prep C18 OBD 5 μm 19*150 mm; Mobile phase: A-water (0.1% TFA): B-acetonitrile; Flow rate: 20 mL/min, the fraction was lyophilized to give 1-2k (trifluoroacetate) (275 mg, 82.8% yield) as a white solid. MS (ESI) m/z: 1131.0 [M+H]⁺.

### Step 3: (2S,3S,4S,5R,6S)-6-(4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10)-dimethyl-3,6,9-trioxo-2,13-dioxa-4,7,10-triazatetradecyl)-2-((R)-7-(2,5-dioxo-2,5-dihydro-1H-pyrrol-1-yl)-2,2-dimethyl-4,11-dioxo-3,10-dioxa-5,12-diazapentadecan-15-amido)phenoxy)-3,4,5-trihydroxytetrahydro-2H-pyran-2-carboxylic acid (1-3b)

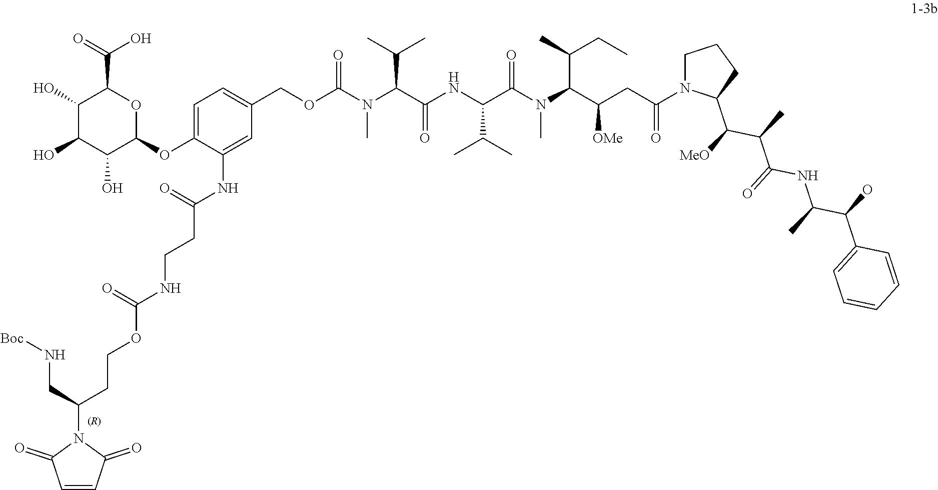

To the solution of 1-2k (trifluoroacetate) (30 mg, 0.024 mmol) in dry DMF (2 mL) were added Int-2 (13 mg, 0.029 mmol) and DIPEA (0.013 mL, 0.072 mmol) at 0° C., stirred at r.t. for 1 h. The resulting solution was purified by prep-HPLC (Method: column XBridge Prep C18 OBD 5 μm 19*150 mm; Mobile phase: A-water (0.1% FA): B-acetonitrile; Flow rate: 20 mL/min, the fraction was lyophilized to give 1-3b (25 mg, 72.0% yield) as a white solid. MS (ESI) m/z: 1440.9 [M+H]⁺.

### Step 4: (2S,3S,4S,5R,6S)-6-(2-(3-((((R)-4-amino-3-(2,5-dioxo-2,5-dihydro-1H-pyrrol-1-yl) butoxy)carbonyl)amino)propanamido)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (1-3) trifluoroacetate

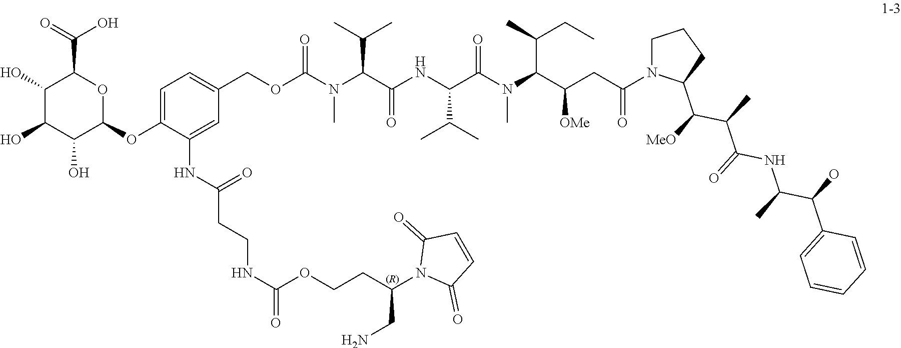

To the solution of 1-3b (25 mg, 0.017 mmol) in CH₂Cl₂ (0.5 mL) was added TFA (0.13 mL, 1.76 mmol) at 0° C., stirred at 0° C. for 30 min. The resulting solution was purified by prep-HPLC (Method: column XBridge Prep C18 OBD 5 μm 19*150 mm; Mobile phase: A-water (0.1% TFA): B-acetonitrile; Flow rate: 20 mL/min, the fraction was lyophilized to give 1-3 (trifluoroacetate) (19 mg, 75.3% yield) as a white solid. MS (ESI) m/z: 1340.8 [M+H]⁺.

### Example 1-4

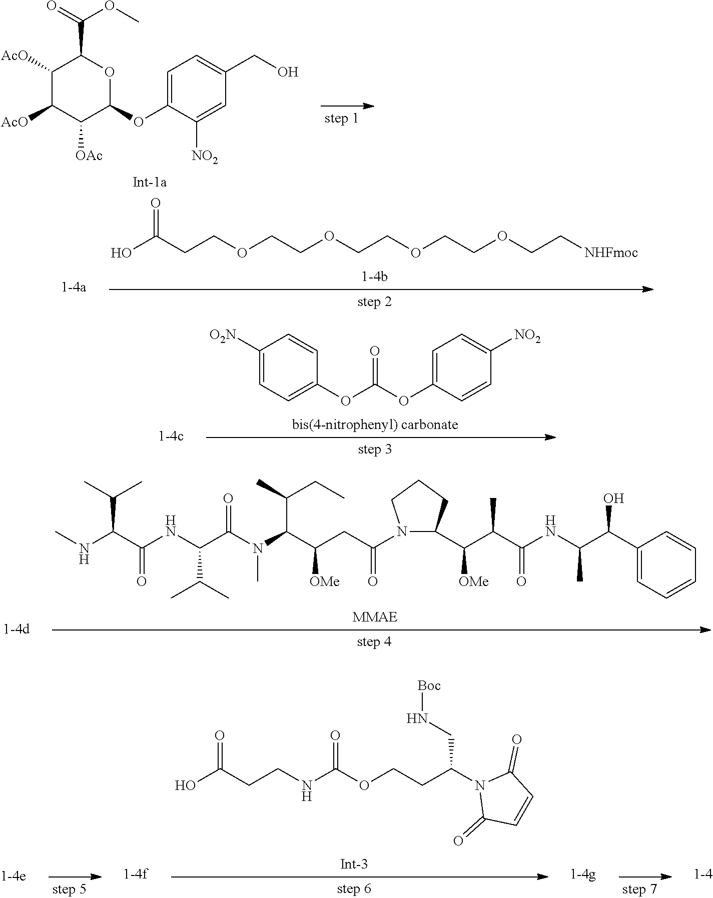

### Step 1: (2S,3R,4S,5S,6S)-2-(2-amino-4-(hydroxymethyl)phenoxy)-6-(methoxycarbonyl)tetrahydro-2H-pyran-3,4,5-triyl triacetate (1-4a)

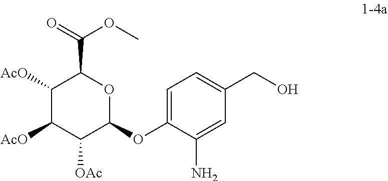 [SMILES: COC(=O)[C@H]1O[C@@H](Oc2ccc(CO)cc2N)[C@H](OC(C)=O)[C@@H](OC(C)=O)[C@@H]1OC(C)=O]

To the solution of Int-1a (500 mg, 1.03 mmol) in EtOAc (10 mL) was wet added Pd/C (75 mg), stirred at r.t. for 16 h under H₂ atmosphere. The solution was filtrated and washes with EtOAc (30 mL) and CH₂Cl (30 mL). The filtrate was concentrated to give the crude product 1-4a (470 mg, crude) as a yellow solid. MS (ESI) m/z: 456.4 [M+H]⁺.

### Step 2: (2S,3R,4S,5S,6S)-2-(2-(1-(9H-fluoren-9-yl)-3-oxo-2,7,10,13,16-pentaoxa-4-azanonadecan-19-amido)-4-(hydroxymethyl)phenoxy)-6-(methoxycarbonyl)tetrahydro-2H-pyran-3,4,5-triyl triacetate (1-4c)

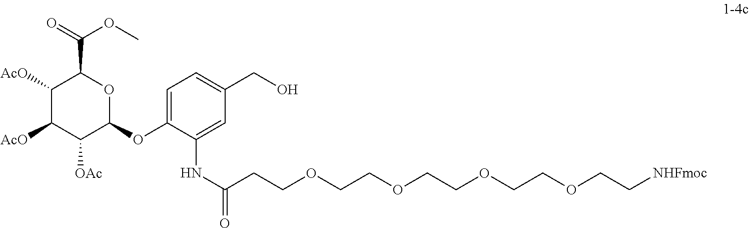

To the solution of 1-4a (95.0 mg, 0.21 mmol) and 1-4b (113 mg, 0.23 mmol) (commercially available) in dry DMF (2 mL) were added HATU (96 mg, 0.25 mmol) and DIPEA (0.074 mL, 0.42 mmol), stirred at r.t. for 2 h. The resulting solution was purified by prep-HPLC (Method: column XBridge Prep C18 OBD 5 μm 19*150 mm; Mobile phase: A-water (0.1% TFA): B-acetonitrile; Flow rate: 20 mL/min, the fraction was lyophilized to give 1-4c (107 mg, 55.4% yield) as a colorless oil. MS (ESI) m/z: 925.7 [M+H]⁺.

### Step 3: (2S,3R,4S,5S,6S)-2-(2-(1-(9H-fluoren-9-yl)-3-oxo-2,7,10,13,16-pentaoxa-4-azanonadecan-19-amido)-4-((((4-nitrophenoxy)carbonyl)oxy)methyl)phenoxy)-6-(methoxycarbonyl)tetrahydro-2H-pyran-3,4,5-triyl triacetate (1-4d)

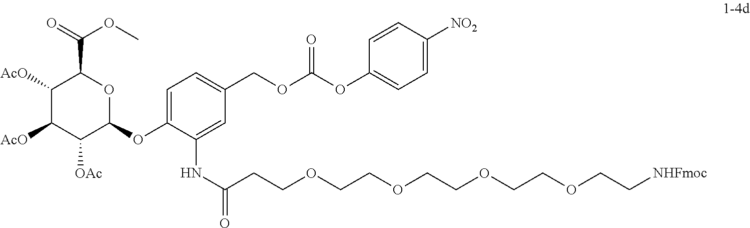

To the solution of 1-4c (107 mg, 0.12 mmol) in dry DMF (2 mL) were added bis(4-nitrophenyl) carbonate (53 mg, 0.17 mmol) and DIPEA (0.037 mL, 0.21 mmol) at 0° C., stirred at r.t. for 6 h. The solution was concentrated under reduced pressure. The residue was purified by silica gel column chromatography (CH₂Cl₂/MeOH=90/10) to give 1-4d (110 mg, 87.2% yield) as an off-white solid. MS (ESI) m/z: 1090.8 [M+H]⁺.

### Step 4: (2S,3R,4S,5S,6S)-2-(2-(1-(9H-fluoren-9-yl)-3-oxo-2,7,10,13,16-pentaoxa-4-azanonadecan-19-amido)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-6-(methoxycarbonyl)tetrahydro-2H-pyran-3,4,5-triyl triacetate (1-4e)

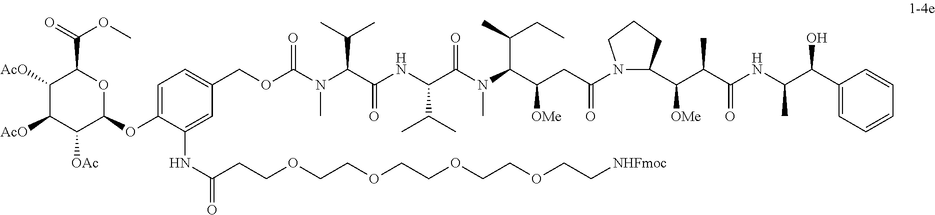

To the solution of 1-4d (110 mg, 0.11 mmol) and MMAE (81 mg, 0.11 mmol) in dry DMF (3 mL) were added HOBt (4.1 mg, 0.03 mmol) and DIPEA (0.033 mL, 0.18 mmol), stirred at r.t. for 16 h. The solution was concentrated under reduced pressure and purified by silica gel column chromatography (CH₂Cl₂/MeOH=90/10) to give 1-4e (90 mg, 76.3% yield) as a white solid. MS (ESI) m/z: 1670.3 [M+H]⁺.

### Step 5: (2S,3S,4S,5R,6S)-6-(2-(1-amino-3,6,9,12-tetraoxapentadecan-15-amido)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (1-4f) trifluoroacetate

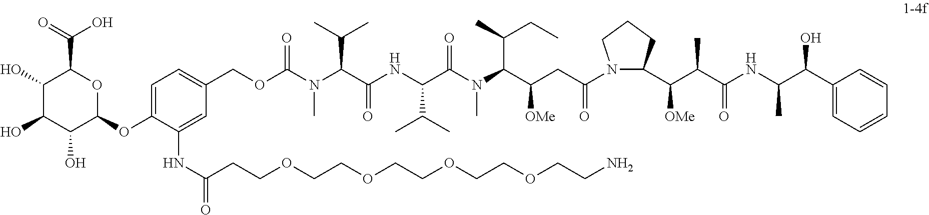 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2O[C@H](C(=O)O)[C@@H](O)[C@H](O)[C@H]2O)c(NC(=O)CCOCCOCCOCCOCCN)c1)C(C)C]

To the solution of 1-4e (90 mg, 0.054 mmol) in THF (1 mL) and water (1 mL) was added 1N LiOH (0.38 mL, 0.38 mmol) at 0° C., stirred at r.t. for 3 h. The pH was adjusted to around 6 by progressively adding 20% aq. AcOH. The resulting solution was purified by prep-HPLC (Method: column XBridge Prep C18 OBD 5 μm 19*150 mm; Mobile phase: A-water (0.1% TFA): B-acetonitrile; Flow rate: 20 mL/min, the fraction was lyophilized to give 1-4f (45 mg, 58.8% yield) (trifluoroacetate) as a white solid. MS (ESI) m/z: 1307.3 [M+H]⁺.

### Step 6: (2S,3S,4S,5R,6S)-6-(4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-((R)-7-(2,5-dioxo-2,5-dihydro-1H-pyrrol-1-yl)-2,2-dimethyl-4,11,15-trioxo-3,10,19,22,25,28-hexaoxa-5,12,16-triazahentriacontan-31-amido)phenoxy)-3,4,5-trihydroxytetrahydro-2H-pyran-2-carboxylic acid (1-4g)

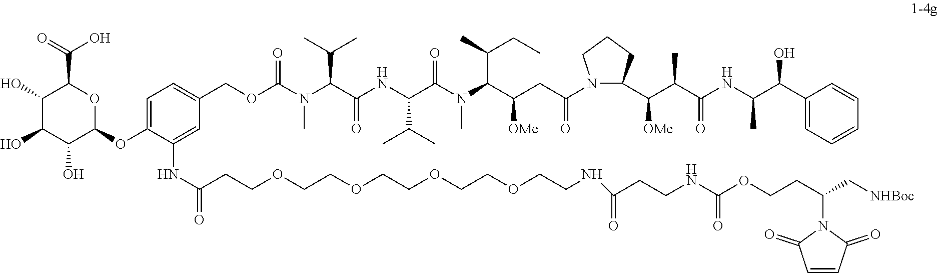 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2O[C@H](C(=O)O)[C@@H](O)[C@H](O)[C@H]2O)c(NC(=O)CCOCCOCCOCCOCCNC(=O)CCNC(=O)OCC[C@H](CNC(=O)OC(C)(C)C)N2C(=O)C=CC2=O)c1)C(C)C]

To the solution of Int-3 (6.8 mg, 0.015 mmol) in dry DMF (0.5 mL) were added HATU (6.5 mg, 0.017 mmol) and DIPEA (0.005 mL, 0.028 mmol), stirred at r.t. for 10 min. Then to the above mixture was added 1-4f (trifluoroacetate) (20 mg, 0.014 mmol), stirred at r.t. for 10 min. The resulting solution was purified by prep-HPLC (Method: column XBridge Prep C18 OBD 5 μm 19*150 mm; Mobile phase: A-water (0.1% TFA): B-acetonitrile: Flow rate: 20 mL/min, the fraction was lyophilized to give 1-4g (15 mg, 63.1% yield) as a white solid. MS (ESI) m/z: 1689.4 [M+H]⁺.

### Step 7: (2,3S,4S,5R,6S)-6-(2-((R)-26-amino-25-(2,5-dioxo-2,5-dihydro-1H-pyrrol-1-yl)-17,21-dioxo-4,7,10,13,22-pentaoxa-16,20-diazahexacosanamido)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (1-4) trifluoroacetate

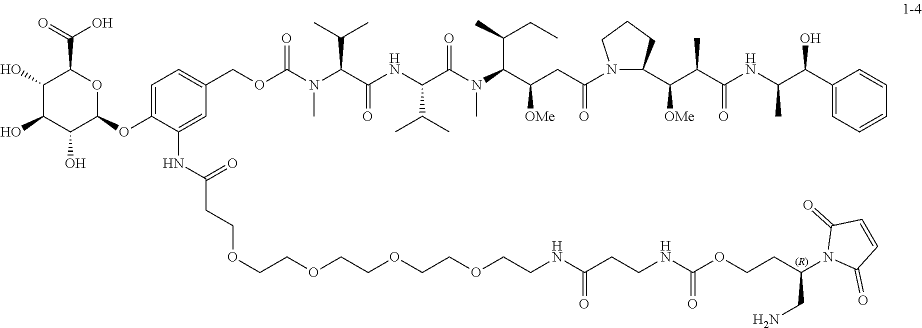

To the solution of 1-4g (15 mg, 0.009 mmol) in CH₂Cl₂ (0.4 mL) was added TFA (0.1 mL, 3.10 mmol) at 0° C., stirred at 0° C. for 30 min. The resulting solution was concentrated under reduced pressure and purified by prep-HPLC (Method: column XBridge Prep C18 OBD 5 μm 19*150 mm; Mobile phase: A-water (0.1% TFA): B-acetonitrile; Flow rate: 20 mL/min, the fraction was lyophilized to give 1-4 (trifluoroacetate) (8.5 mg, 56.2% yield) as a white solid. MS (ESI) m/z: 1589.3 [M+H]⁺.

### Example 1-5

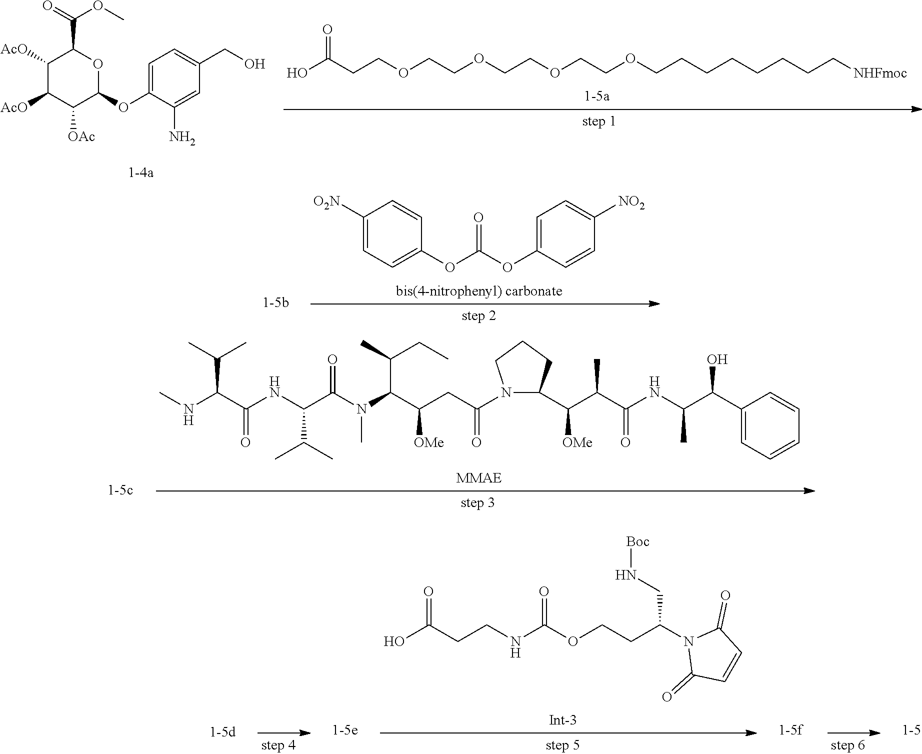

### Step 1: (2S,3R,4S,5S,6S)-2-(2-(1-(9H-fluoren-9-yl)-3-oxo-2,7,10,13,16,19,22-heptaoxa-4-azapentacosan-25-amido)-4-(hydroxymethyl)phenoxy)-6-(methoxycarbonyl)tetrahydro-2H-pyran-3,4,5-triyl triacetate (1-5b)

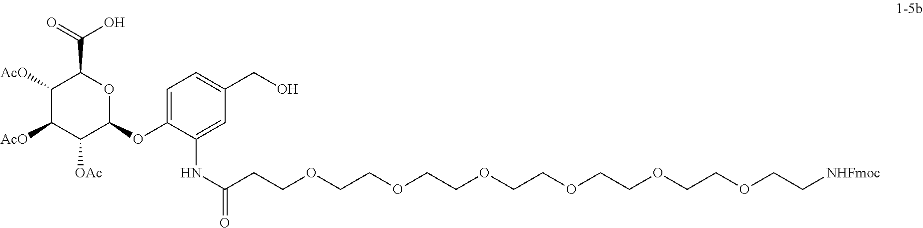

To the solution of 1-4a (80 mg, 0.18 mmol) and 1-5a (112 mg, 0.19 mmol) in dry DMF (1 mL) were added HATU (81 mg, 0.21 mmol) and DIPEA (0.062 mL, 0.35 mmol), stirred at r.t. for 20 min. The resulting solution was purified by prep-HPLC (Method: column XBridge Prep C18 OBD 5 um 19*150 mm; Mobile phase: A-water (0.1% TFA): B-acetonitrile; Flow rate: 20 mL/min, the fraction was lyophilized to give 1-5b (80 mg, 44.9% yield) as colorless oil. MS (ESI) m/z: 1035.7 [M+Na]⁺

### Step 2: (2S,3R,4S,5S,6S)-2-(2-(1-(9H-fluoren-9-yl)-3-oxo-2,7,10,13,16,19,22-heptaoxa-4-azapentacosan-25-amido)-4-((((4-nitrophenoxy)carbonyl)oxy)methyl)phenoxy)-6-(methoxycarbonyl)tetrahydro-2H-pyran-3,4,5-triyl triacetate (1-5c)

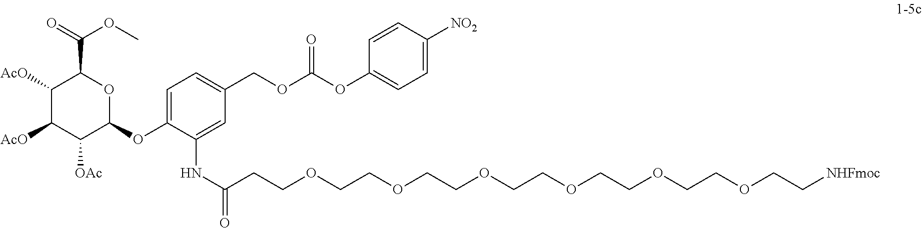

To the solution of 1-5b (80 mg, 0.079 mmol) in dry DMF (2 mL) were added bis(4-nitrophenyl) carbonate (36 mg, 0.12 mmol) and DIPEA (0.025 mL, 0.14 mmol) at 0° C., stirred at r.t. for 6 h. The solution was concentrated under reduced pressure. The residue was purified by silica gel column chromatography (CH₂Cl/MeOH=90/10) to give 1-5c (80 mg, 86% yield) as an off-white solid. MS (ESI) m/z: 1178.8 [M+H]⁺.

### Step 3: (2S,3R,4S,5S,6S)-2-(2-(1-(9H-fluoren-9-yl)-3-oxo-2,7,10,13, 16, 19,22-heptaoxa-4-azapentacosan-25-amido)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-6-(methoxycarbonyl)tetrahydro-2H-pyran-3,4,5-triyl triacetate (1-5d)

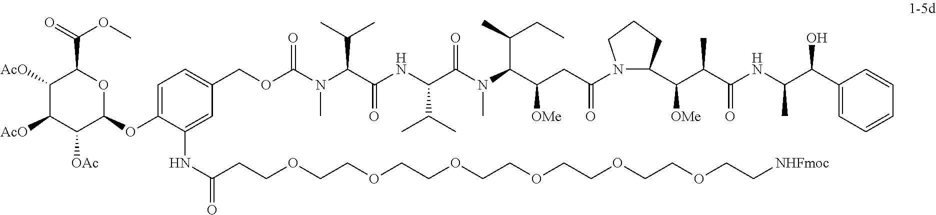

To the solution of 1-5c (80 mg, 0.068 mmol) and MMAE (59 mg, 0.081 mmol) in dry DMF (2 mL) were added HOBt (2.8 mg, 0.020 mmol) and DIPEA (0.022 mL, 0.12 mmol), stirred at r.t. for 16 h. The resulting solution was concentrated and purified by silica gel column chromatography (CH₂Cl₂/MeOH=90/10) to give 1-5d (80 mg, 67.1% yield) as a white solid. MS (ESI) m/z: 1758.3 [M+H]⁺.

### Step 4: (2S,3S,4S,5R,6S)-6-(2-(1-amino-3,6,9,12,15,18-hexaoxahenicosan-21-amido)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (1-5e) trifluoroacetate

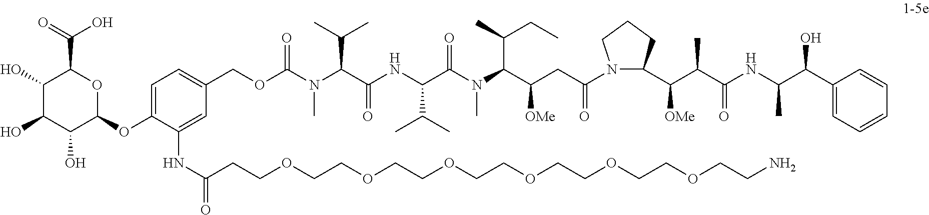 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2O[C@H](C(=O)O)[C@@H](O)[C@H](O)[C@H]2O)c(NC(=O)CCOCCOCCOCCOCCOCCOCCN)c1)C(C)C]

To the solution of 1-5d (80 mg, 0.046 mmol) in THF (1 mL) and water (1 mL) was added 1N LiOH (0.32 mL, 0.32 mmol) at 0° C., stirred at r.t. for 3 h. The pH was adjusted to around 6 by progressively adding 20% aq. AcOH. The resulting solution was purified by prep-HPLC (Method: column XBridge Prep C18 OBD 5 um 19*150 mm; Mobile phase: A-water (0.1% TFA): B-acetonitrile; Flow rate: 20 mL/min, the fraction was lyophilized to give 1-5e (trifluoroacetate) (40 mg, 63.0% yield) as a white solid. MS (ESI) m/z: 1395.2 [M+H]⁺.

### Step 5: (2S,3S,4S,5R,6S)-6-(4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-((R)-7-(2,5-dioxo-2,5-dihydro-1H-pyrrol-1-yl)-2,2-dimethyl-4,11,15-trioxo-3,10,19,22,25,28,31,34-octaoxa-5,12,16-triazaheptatriacontan-37-amido)phenoxy)-3,4,5-trihydroxytetrahydro-2H-pyran-2-carboxylic acid (1-5f)

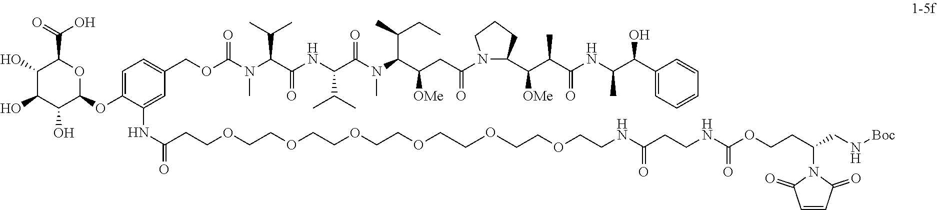 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2O[C@H](C(=O)O)[C@@H](O)[C@H](O)[C@H]2O)c(NC(=O)CCOCCOCCOCCOCCOCCOCCNC(=O)CCNC(=O)OCC[C@H](CNC(=O)OC(C)(C)C)N2C(=O)C=CC2=O)c1)C(C)C]

To the solution of Int-3 (5.2 mg, 0.013 mmol) in dry DMF (0.5 mL) were added HATU (5.0 g, 0.013 mmol) and DIPEA (0.004 mL, 0.022 mmol), stirred at r.t. for 10 min. Then to the above mixture was added 1-5e (trifluoroacetate) (15 mg, 0.011 mmol), stirred at r.t. for 10 min. The resulting solution was purified by prep-HPLC (Method: column XBridge Prep C18 OBD 5 μm 19*150 mm; Mobile phase: A-water (0.1% TFA): B-acetonitrile; Flow rate: 20 mL/min, the fraction was lyophilized to give 1-5f (9.0 mg, 47.1% yield) as a white solid. MS (ESI) m/z: 1777.4 [M+H]⁺.

### Step 6: (2S,3S,4S,5R,6S)-6-(2-((R)-32-amino-31-(2,5-dioxo-2,5-dihydro-1H-pyrrol-1-yl)-23,27-dioxo-4,7,10,13,16,19,28-heptaoxa-22,26-diazadotriacontanamido)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (1-5) trifluoroacetate

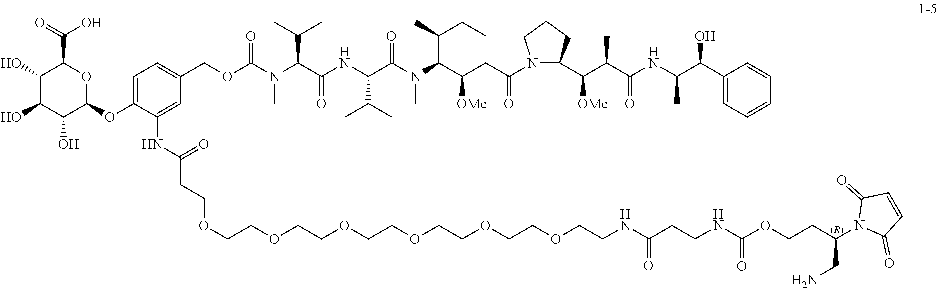

To the solution of 1-5f (9.0 mg, 0.005 mmol) in CH₂Cl₂ (0.8 mL) was added TFA (0.2 mL, 1.77 mmol) at 0° C., stirred at 0° C. for 30 min. The resulting solution was concentrated and purified by prep-HPLC (Method: column XBridge Prep C18 OBD 5 μm 19*150 mm; Mobile phase: A-water (0.1% TFA): B-acetonitrile; Flow rate: 20 mL/min, the fraction was lyophilized to give 1-5 (trifluoroacetate) (6.9 mg, 81.2% yield) as a white solid. MS (ESI) m/z: 1676.7 [M+H]⁺.

### Example 1-6

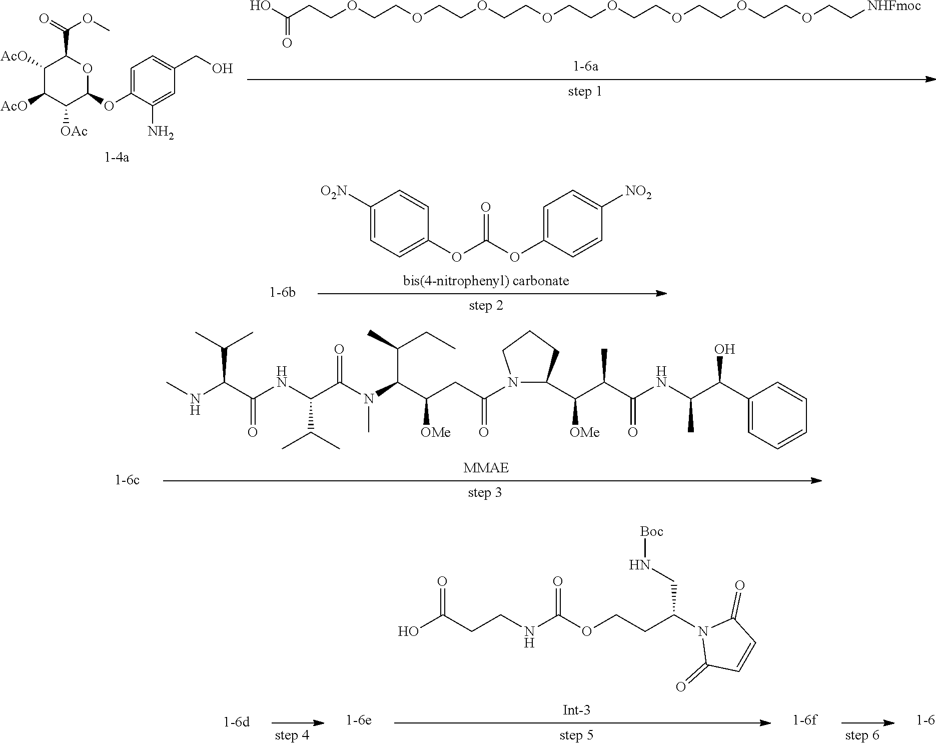

### Step 1: (2S,3R,4S,5S,6S)-2-(2-(1-(9H-fluoren-9-yl)-3-oxo-2,7,10,13,16,19,22,25,28-nonaoxa-4-azahentriacontan-31-amido)-4-(hydroxymethyl)phenoxy)-6-(methoxycarbonyl)tetrahydro-2H-pyran-3,4,5-triyl triacetate (1-6b)

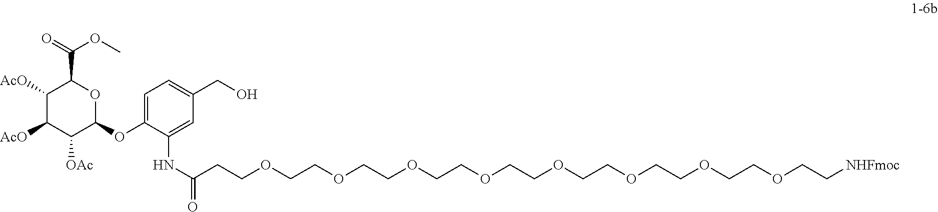

To the solution of 1-4b (50 mg, 0.11 mmol) and 1-6a (77 mg, 0.12 mmol) in dry DMF (1 mL) were added HATU (51 mg, 0.13 mmol) and DIPEA (0.062 mL, 0.35 mmol), stirred at r.t. for 20 min. The resulting solution was purified by prep-HPLC (Method: column XBridge Prep C18 OBD 5 μm 19*150 mm; Mobile phase: A-water (0.1% TFA): B-acetonitrile; Flow rate: 20 mL/min, the fraction was lyophilized to give 1-6b (44 mg, 36.4% yield) as a colorless oil. MS (ESI) m/z: 1102.8 [M+H]⁺.

### Step 2: (2S,3R,4S,5S,6S)-2-(2-(1-(9H-fluoren-9-yl)-3-oxo-2,7,10,13,16,19,22,25,28-nonaoxa-4-azahentriacontan-31-amido)-4-((((4-nitrophenoxy)carbonyl)oxy)methyl)phenoxy)-6-(methoxycarbonyl)tetrahydro-2H-pyran-3,4,5-triyl triacetate (1-6c)

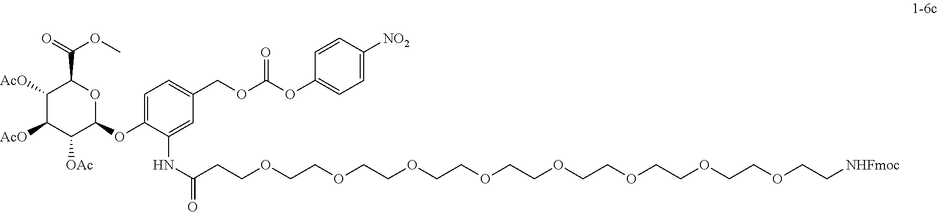

To the solution of 1-6b (44 mg, 0.04 mmol) in dry DMF (I mL) were added bis(4-nitrophenyl) carbonate (18 mg, 0.060 mmol) and DIPEA (0.013 mL, 0.072 mmol) at 0° C., stirred at r.t. for 6 h. The solution was concentrated under reduced pressure. The residue was purified by silica column gel chromatography (CH₂Cl/MeOH=90/10) to give 1-6c (45 mg, 88.9% yield). MS (ESI) m/z: 1266.9 [M+H]⁺.

### Step 3: (2S,3R,4S,5S,6S)-2-(2-(1-(9H-fluoren-9-yl)-3-oxo-2,7,10,13,16,19,22,25,28-nonaoxa-4-azahentriacontan-31-amido)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-6-(methoxycarbonyl)tetrahydro-2H-pyran-3,4,5-triyl triacetate (1-6d)

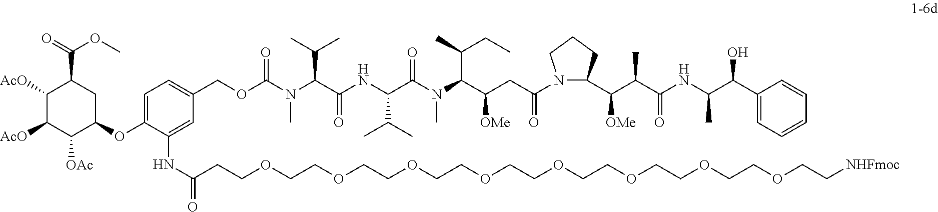

To the solution of 1-6c (45 mg, 0.036 mmol) and MMAE (34 mg, 0.046 mmol) in dry DMF (2 mL) were added HOBt (1.5 mg, 0.011 mmol) and DIPEA (0.019 mL, 0.11 mmol), stirred at r.t. for 16 h. The resulting solution was concentrated and purified by silica gel column chromatography (CH₂Cl₂/MeOH=90/10) to give 1-6d (50 mg, 76.3% yield) as a white solid. MS (ESI) m/z: 1845.9 [M+H]⁺.

### Step 4: (2S,3S,4S,5R,6S)-6-(2-(1-amino-3,6,9,12,15,18,21,24-octaoxaheptacosan-27-amido)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (1-6e) trifluoroacetate

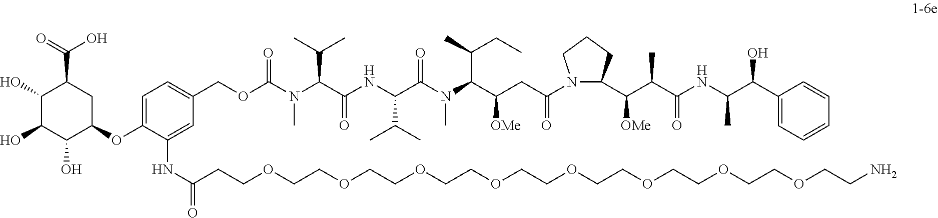 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2C[C@H](C(=O)O)[C@@H](O)[C@H](O)[C@H]2O)c(NC(=O)CCOCCOCCOCCOCCOCCOCCOCCOCCN)c1)C(C)C]

To the solution of 1-3d (50 mg, 0.027 mmol) in THF (1 mL) and water (1 mL) was added 1N LiOH (0.19 mL, 0.190 mmol) at 0° C., stirred at r.t. for 3 h. The pH was adjusted to around 6 by progressively adding 20% aq. AcOH. The resulting solution was purified by prep-HPLC (Method: column XBridge Prep C18 OBD 5 μm 19*150 mm; Mobile phase: A-water (0.1% TFA): B-acetonitrile; Flow rate: 20 mL/min, the fraction was lyophilized to give 1-6e (trifluoroacetate) (25 mg, 62.2% yield) as a white solid. MS (ESI) m/z: 1483.6 [M+H]⁺.

### Step 5: (2S,3S,4S,5R,6S)-6-(4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-((R)-7-(2,5-dioxo-2,5-dihydro-1H-pyrrol-1-yl)-2,2-dimethyl-4,11,15-trioxo-3,10,19,22,25,28,31,34,37,40-decaoxa-5,12,16-triazatritetracontan-43-amido)phenoxy)-3,4,5-trihydroxytetrahydro-2H-pyran-2-carboxylic acid (1-6f)

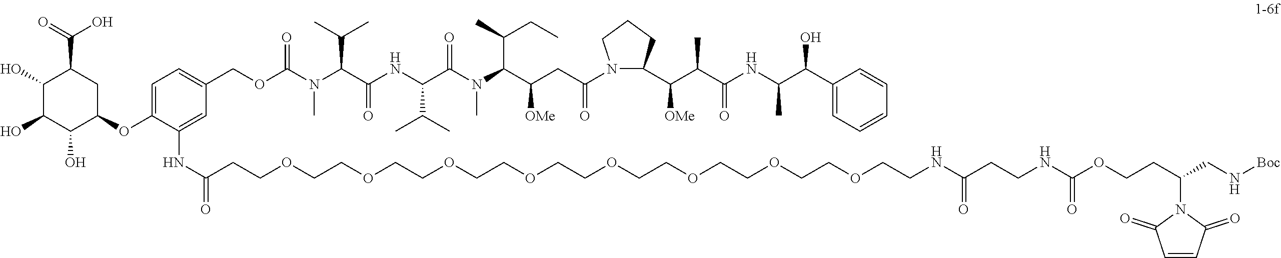 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2C[C@H](C(=O)O)[C@@H](O)[C@H](O)[C@H]2O)c(NC(=O)CCOCCOCCOCCOCCOCCOCCOCCOCCNC(=O)CCNC(=O)OCC[C@H](CNC(=O)OC(C)(C)C)N2C(=O)C=CC2=O)c1)C(C)C]

To the solution of Int-3 (6.1 mg, 0.015 mmol) in dry DMF (0.5 mL) were added HATU (5.8 mg, 0.015 mmol) and DIPEA (0.004 mL, 0.025 mmol) stirred at r.t. for 10 min. Then to the above mixture was added 1-6e (trifluoroacetate) (20 mg, 0.013 mmol), stirred at r.t. for 10 min. The resulting solution was purified by prep-HPLC (Method: column XBridge Prep C18 OBD 5 μm 19*150 mm; Mobile phase: A-water (0.1% FA): B-acetonitrile: Flow rate: 20 mL/min, the fraction was lyophilized to give 1-6f (11 mg, 47.1% yield) as a white solid. MS (ESI) m/z: 1865.4 [M+H]⁺.

### Step 6: (2S,3S,4S,5R,6S)-6-(2-((R)-38-amino-37-(2,5-dioxo-2,5-dihydro-1H-pyrrol-1-yl)-29,33-dioxo-4,7,10,13,16,19,22,25,34-nonaoxa-28,32-diazaoctatriacontanamido)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (1-6) trifluoroacetate

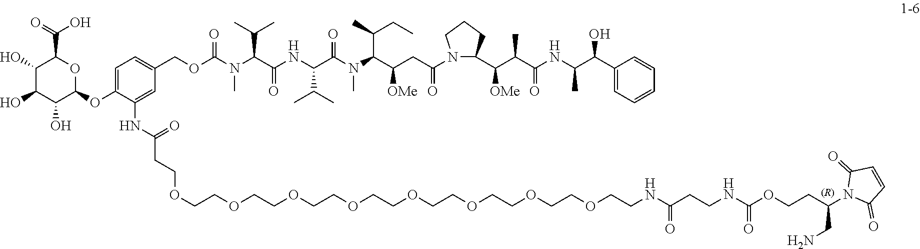

To the solution of 1-6f (11 mg, 0.006 mmol) in CH₂Cl₂ (0.8 mL) was added TFA (0.2 mL, 1.774 mmol) at 0° C., stirred at 0° C. for 30 min. The resulting solution was concentrated and purified by prep-HPLC (Method: column XBridge Prep C18 OBD 5 um 19*150 mm; Mobile phase: A-water (0.1% TFA): B-acetonitrile; Flow rate: 20 mL/min, the fraction was lyophilized to give 1-6 (trifluoroacetate) (7.8 mg, 70.4% yield) as a white solid. MS (ESI) m/z: 1764.8 [M+H]⁺.

### Example 1-7

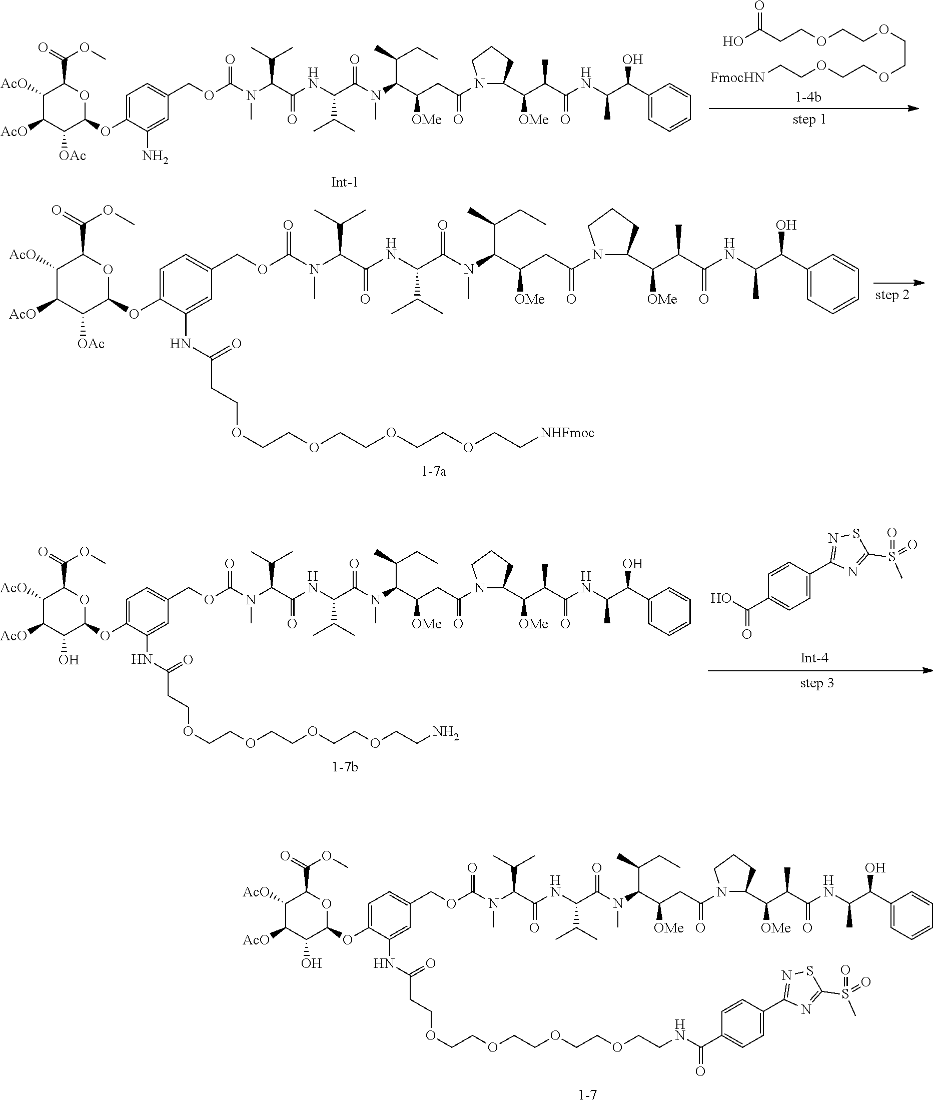

1-7 (7.5 mg) was obtained according to the similar procedure of Example 1-8. MS (ESI) m/z: 1573.5 [M+H]⁺.

### Example 1-8

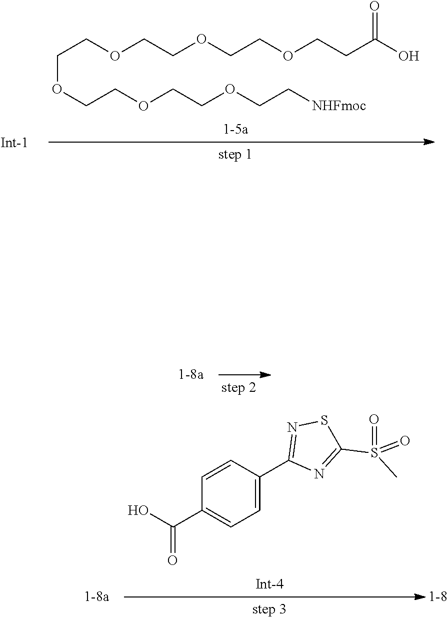

### Step 1: (2S,3R,4S,5S,6S)-2-(2-(1-(9H-fluoren-9-yl)-3-oxo-2,7,10,13,16,19,22-heptaoxa-4-azapentacosan-25-amido)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-6-(methoxycarbonyl)tetrahydro-2H-pyran-3,4,5-triyl triacetate (1-8a)

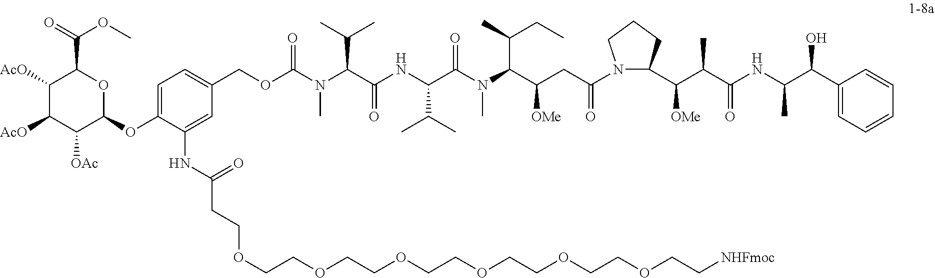

To the solution of Int-1 (100 mg, 0.083 mmol). 1-5a (54 mg, 0.092 mmol) and HATU (39 mg, 0.1 mmol) in dry DMF (1 mL) was added 2,6-lutidine (25 μL, 0.21 mmol), stirred at r.t. for 17 h. The solution was added into 0.5 N HCl (1 mL) and water (10 mL), extracted with CH₂Ch/MeOH (10:1, v/v, 11 mL*4). The organic phase was concentrated and purified by flash column chromatography (10% MeOH in CH₂Cl₂/CH₂Cl₂=50/50) to give the compound 1-8a as a colorless oil (130 mg, 88.7% yield). MS (ESI) m/z: 1756.9 [M+H]⁺.

### Step 2: (2S,3S,4S,5R,6S)-6-(2-(1-amino-3,6,9,12,15,18-hexaoxahenicosan-21-amido)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (1-8b)

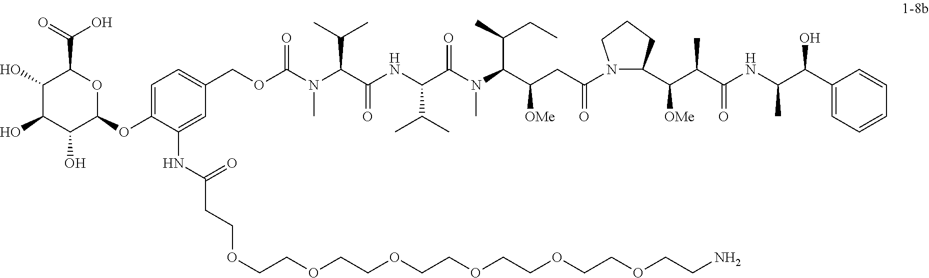 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2O[C@H](C(=O)O)[C@@H](O)[C@H](O)[C@H]2O)c(NC(=O)CCOCCOCCOCCOCCOCCOCCN)c1)C(C)C]

To the compound 1-8a (120 mg, 0.068 mmol) was added a solution of LiBr (359 mg, 4.1 mmol) in MeCN/H₂O (1.7/0.17 mL), followed by addition of TEA (58 μL, 0.41 mmol), stirred at r.t. for 3 h. HOAc was added to adjust pH to 6. The solution was concentrated, the residue was dissolved in dry DMF (2 mL). Et₂NH (144 μL, 1.37 mmol) was added, stirred at r.t. for another 1 h. The solution was concentrated and purified by prep-HPLC (Column: Xbridge Prep C18 OBD™ 5 μm, 19*150 mm; Mobile phase A: 0.1% FA in water, B: MeCN; Gradient: 20%-60% B; Flow rate: 20 mL/min), lyophilized to give the compound 1-8b as a white solid (69 mg, 72.4% yield). MS (ESI) m/z: 1394.8 [M+H]⁺.

### Step 3: (2S,3S,4S,5R,6S)-6-(4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-(1-(4-(5-(methylsulfonyl)-1,2,4-thiadiazol-3-yl)phenyl)-1-oxo-5,8,11,14,17,20-hexaoxa-2-azatricosan-23-amido)phenoxy)-3,4,5-trihydroxytetrahydro-2H-pyran-2-carboxylic acid (1-8)

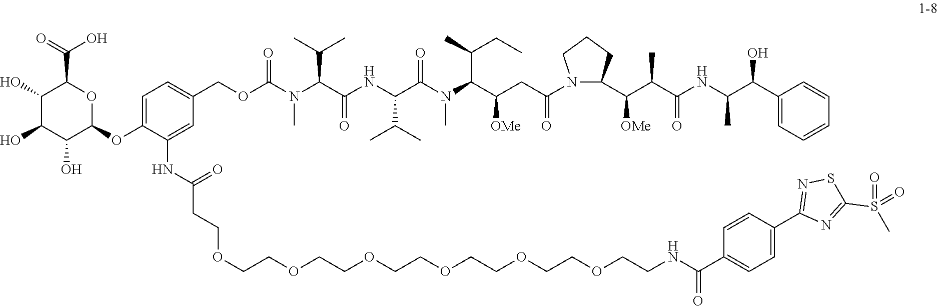 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2O[C@H](C(=O)O)[C@@H](O)[C@H](O)[C@H]2O)c(NC(=O)CCOCCOCCOCCOCCOCCOCCNC(=O)c2ccc(-c3nsc(S(C)(=O)=O)n3)cc2)c1)C(C)C]

To the solution of Int-4 (4.6 mg, 0.016 mmol) and HATU (6.1 mg, 0.016 mmol) in dry DMF (0.5 mL) was added DIEA (3 μL, 0.017 mmol), stirred at r.t. for 15 min. The solution was added to the solution of 1-8b (20 mg, 0.014 mmol) in dry DMF (0.5 mL), DIEA (2 μL, 0.012 mmol) was added, stirred at r.t. for 10 min. The solution was purified by prep-HPLC (Column: Xbridge Prep C18 OBD™ 5 μm, 19*150 mm; Mobile phase A: 0.1% FA in water, B: MeCN; Gradient: 30%-65% B; Flow rate: 20 mL/min), lyophilized to give the compound 1-8 as a white solid (13.4 mg, 56.3% yield). MS (ESI) m/z: 1661.2 [M+H]⁺.

### Example 1-9

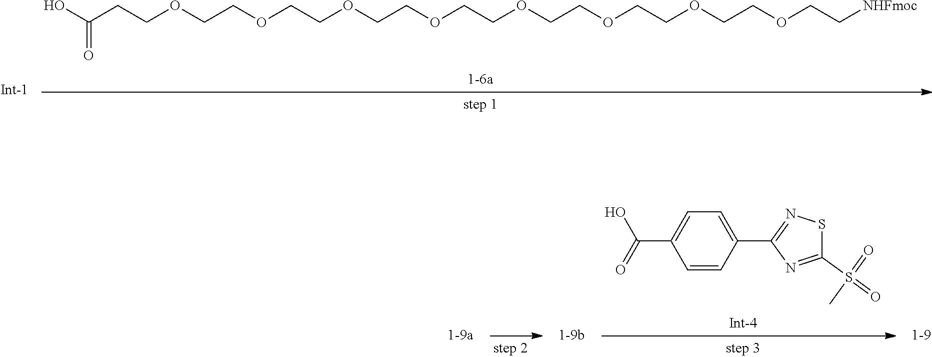

### Step 1: (2S,3R,4S,5S,6S)-2-(2-(1-(9H-fluoren-9-yl)-3-oxo-2,7,10,13,16,19,22,25,28-nonaoxa-4-azahentriacontan-31-amido)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-6-(methoxycarbonyl)tetrahydro-2H-pyran-3,4,5-triyl triacetate (1-9a)

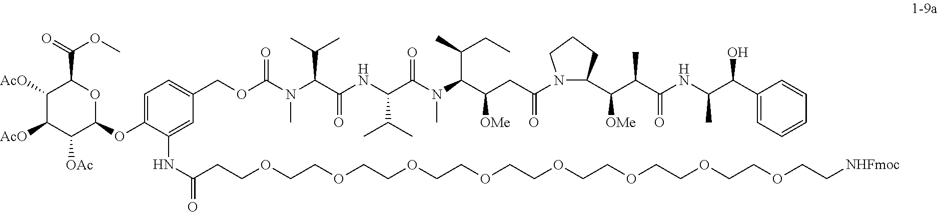

To the solution of Int-1 (50 mg, 0.042 mmol) and 1-6a (31 mg, 0.047 mmol) in dry DMF (1 mL) were added HATU (19 mg, 0.050 mmol) and DIPEA (0.015 mL, 0.13 mmol), stirred at r.t. for 18 h. The resulting solution was quenched with sat. NH₄Cl, extracted with EtOAc. The organic phase was concentrated and purified by silica gel column chromatography (CH₂Cl₂/MeOH=98/2) to give 1-9a (50 mg, 65.0% yield) as a white solid. MS (ESI) m/z: 1845.4 [M+H]⁺.

### Step 2: (2S,3S,4S,5R,6S)-6-(2-(1-amino-3,6,9,12,15,18,21,24-octaoxaheptacosan-27-amido)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (1-9b) trifluoroacetate

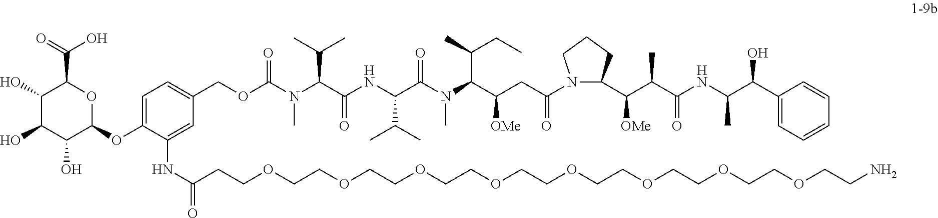 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2O[C@H](C(=O)O)[C@@H](O)[C@H](O)[C@H]2O)c(NC(=O)CCOCCOCCOCCOCCOCCOCCOCCOCCN)c1)C(C)C]

To the solution of 1-9a (50 mg, 0.027 mmol) in water (0.5 mL) and THF (0.5 mL) was added 1N LiOH (0.19 mL, 0.19 mmol) at 0° C., stirred at r.t. for 3 h. The pH was adjusted to around 4 by progressively adding formic acid. The resulting solution was purified by prep-HPLC (Method: column XBridge Prep C18 OBD 5 μm 19*150 mm; Mobile phase: A-water (0.1% TFA): B-acetonitrile; Flow rate: 20 mL/min, the fraction was lyophilized to give 1-9b (trifluoroacetate) (20 mg, 49.8% yield) as a white solid. MS (ESI) m/z: 1483.6 [M+H]⁺.

### Step 3: (2S,3R,4S,5S,6S)-2-(2-amino-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-6-(methoxycarbonyl)tetrahydro-2H-pyran-3,4,5-triyl triacetate (1-9)

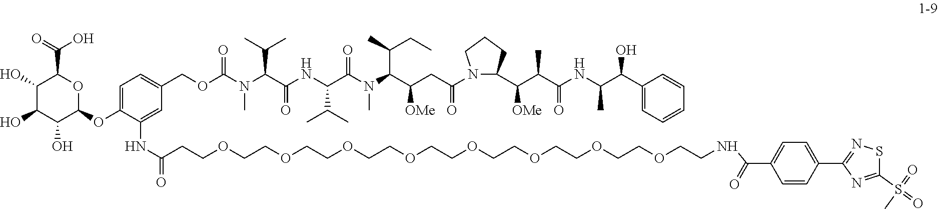 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2O[C@H](C(=O)O)[C@@H](O)[C@H](O)[C@H]2O)c(NC(=O)CCOCCOCCOCCOCCOCCOCCOCCOCCNC(=O)c2ccc(-c3nsc(S(C)(=O)=O)n3)cc2)c1)C(C)C]

To the solution of Int-4 (4.3 mg, 0.015 mmol) in dry DMF (0.5 mL) were added HATU (5.8 mg, 0.015 mmol) and DIPEA (0.0084 mL, 0.025 mmol), stirred at r.t. for 15 min. Then to the above mixture was added 1-9b (trifluoroacetate) (20 mg, 0.013 mmol), stirred at r.t. for 10 min. The resulting solution was purified by prep-HPLC (Method: column XBridge Prep C18 OBD 5 um 19*150 mm; Mobile phase: A-water (0.1% TFA): B-acetonitrile; Flow rate: 20 mL/min, the fraction was lyophilized to give 1-9 (14 mg, 63.9% yield) as a white solid. MS (ESI) m/z: 1750.3 [M+H]⁺.

### Example 1-10

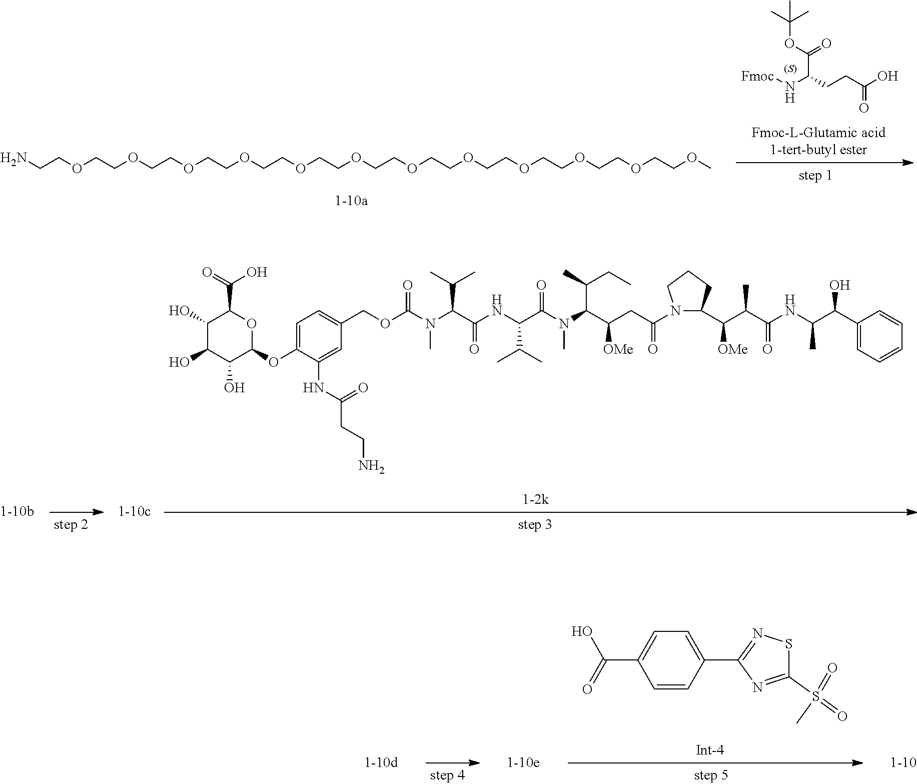

### Step 1: tert-butyl(S)-42-((((9H-fluoren-9-yl)methoxy)carbonyl)amino)-39-oxo-2,5,8,11,14,17,20,23,26,29,32,35-dodecaoxa-38-azatritetracontan-43-oate (1-10b)

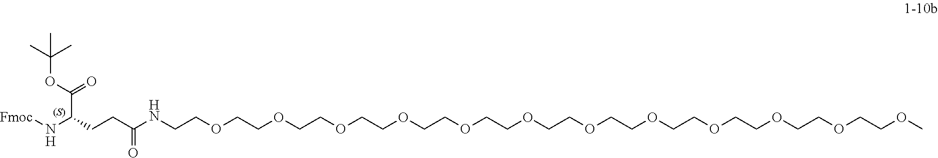

To the solution of Fmoc-L-Glutamic acid 1-tert-butyl ester (69 mg, 0.16 mmol) and 1-10a (90 mg, 0.16 mmol) in dry DMF (2 mL) were added HATU (74 mg, 0.19 mmol) and DIPEA (0.058 mL, 0.32 mmol), stirred at r.t. for 20 min. The resulting solution was quenched with sat. NH₄Cl, extracted with EtOAc (30 mL*3). The organic phase was concentrated and purified by silica gel column chromatography (CH₂Cl₂/MeOH=98/2) to give 1-10b (130 mg, 83.6% yield) as a colorless oil. MS (ESI) m/z: 967.9 [M+H]⁺.

### Step 2: (S)-42-((((9H-fluoren-9-yl)methoxy)carbonyl)amino)-39-oxo-2,5,8,11,14,17,20,23,26,29,32,35-dodecaoxa-38-azatritetracontan-43-oic acid (1-10c)

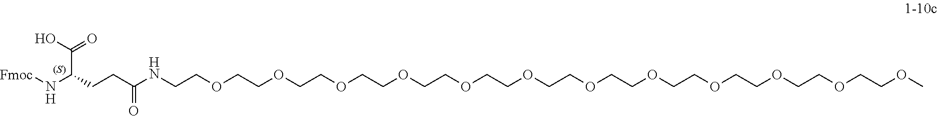

To the solution of 1-10b (60 mg, 0.062 mmol) in CH₂Cl₂ (0.4 mL) was added TFA (0.19 mL, 2.48 mmol), stirred at r.t. for 3 h. The resulting solution was concentrated under vacuo and co-evaporated with toluene five times to give 1-10c (55 mg, crude) as a colorless oil, which directly used in the next step. MS (ESI) m/z: 911.9 [M+H]⁺.

### Step 3: (2S,3S,4S,5R,6S)-6-(2-((S)-42-((((9H-fluoren-9-yl)methoxy)carbonyl)amino)-39,43-dioxo-2,5,8,11,14,17,20,23,26,29,32,35-dodecaoxa-38,44-diazaheptatetracontan-47-amido)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (1-10d)

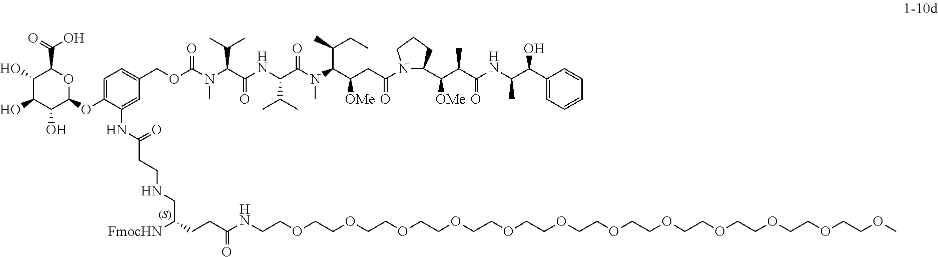

To the solution of 1-10c (16 mg, 0.018 mmol) in dry DMF (0.5 mL) were added HATU (7.4 mg, 0.019 mmol) and DIPEA (0.009 mL, 0.048 mmol), stirred at r.t. for 10 min. Then to the above mixture was added 1-2k (20 mg, 0.016 mmol) at 0° C., stirred at r.t. for 10 min. The resulting solution was purified by prep-HPLC (Method: column XBridge Prep C18 OBD 5 μm 19*150 mm; Mobile phase: A-water (0.1% FA): B-acetonitrile: Flow rate: 20 mL/min, the fraction was lyophilized to give 1-10d (13 mg, 40.0% yield) as a white solid. MS (ESI) m/z: 2024.1 [M+H]⁺.

### Step 4: (2S,3S,4S,5R,6S)-6-(2-((S)-42-amino-39,43-dioxo-2,5,8,11,14,17,20,23,26,29,32,35-dodecaoxa-38,44-diazaheptatetracontan-47-amido)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (1-10e)

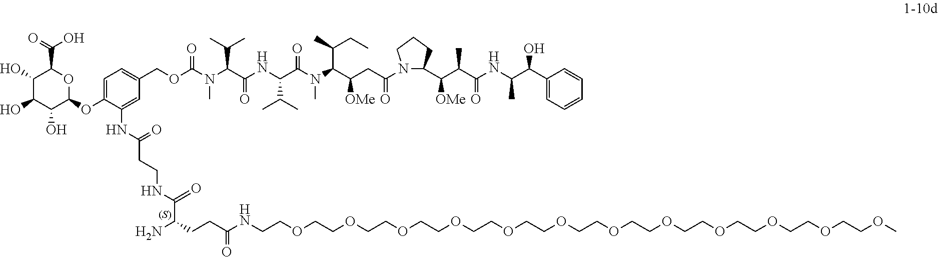

To the solution of 1-10d (13 mg, 0.006 mmol) in dry DMF (1 mL) was added Et₂NH (0.02 mL, 0.19 mmol), stirred at r.t. for 20 min. 1-10e (7 mg, crude) was co-evaporated with toluene five times to remove solvent. MS (ESI) m/z: 1801.9 [M+H]⁺.

### Step 5: (2S,3S,4S,5R,6S)-6-(4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-((S)-42-(4-(5-(methylsulfonyl)-1,2,4-thiadiazol-3-yl)benzamido)-39,43-dioxo-2,5,8,11,14,17,20,23,26,29,32,35-dodecaoxa-38,44-diazaheptatetracontan-47-amido)phenoxy)-3,4,5-trihydroxytetrahydro-2H-pyran-2-carboxylic acid (1-10)

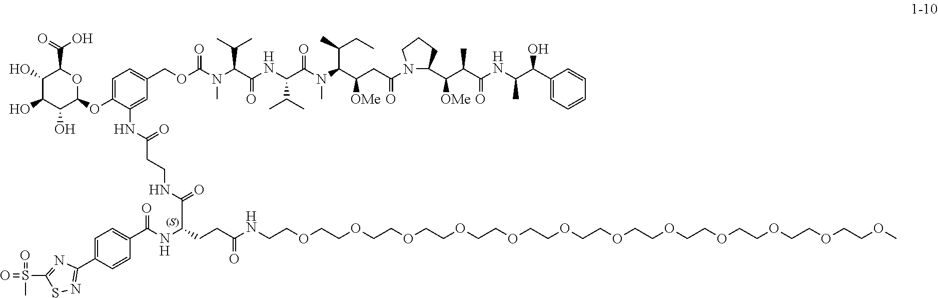

To the solution of Int-4 (2.0 mg, 0.007 mmol) in dry DMF (1 mL) were added HATU (3.0 mg, 0.008 mmol) and DIPEA (0.002 mL, 0.013 mmol), stirred at r.t. for 15 min. Then to the above mixture was added 1-10e (7.0 mg, 0.006 mmol), stirred at r.t. for 10 min. The resulting solution was purified by prep-HPLC (Method: column XBridge Prep C18 OBD 5 um 19*150 mm; Mobile phase: A-water (0.1% TFA): B-acetonitrile; Flow rate: 20 mL/min, the fraction was lyophilized to give 1-10 (5.2 mg, 38.8% yield) as a white solid. MS (ESI) m/z: 2068.1 [M+H]⁺.

### Example 1-11

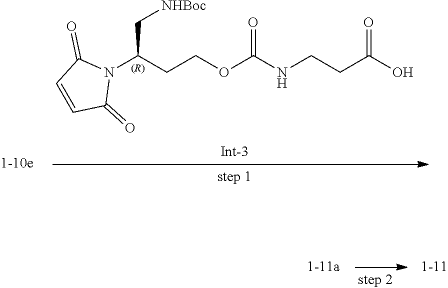

### Step 1: (2S,3S,4S,5R,6S)-6-(2-((S)-42-amino-39,43-dioxo-2,5,8,11,14,17,20,23,26,29,32,35-dodecaoxa-38,44-diazaheptatetracontan-47-amido)-4-((5S,8S,11S,12R)-11-((S)-sec-butyl)-12-(2—((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (1-11a)

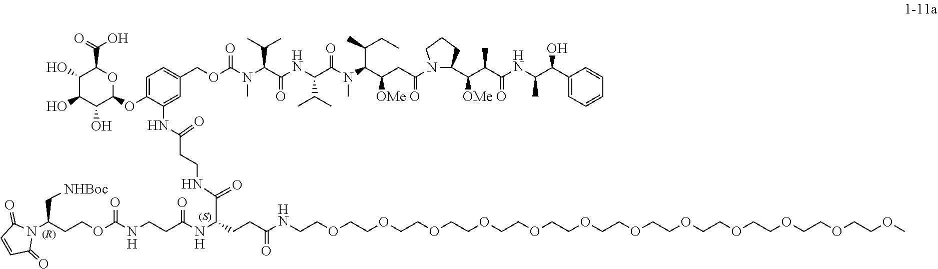

To the solution of Int-3 (18 mg, 0.007 mmol) in dry DMF (1 mL) were added HATU (17 mg, 0.044 mmol) and DIPEA (0.013 mL, 0.074 mmol), stirred at r.t. for 10 min. Then to the above mixture was added 1-10e (40 mg, 0.037 mmol), stirred at r.t. for 10 min. The resulting solution was purified by prep-HPLC (Method: column XBridge Prep C18 OBD 5 um 19*150 mm; Mobile phase: A-water (0.1% FA): B-acetonitrile; Flow rate: 20 mL/min, the fraction was lyophilized to give 1-11a (50 mg, 61.9% yield) as a white solid. MS (ESI) m/z: 2183.1 [M+H]⁺.

### Step 2: (2S,3S,4S,5R,6S)-6-(2-((S)-42-(3-((((R)-4-amino-3-(2,5-dioxo-2,5-dihydro-1H-pyrrol-1-yl) butoxy)carbonyl)amino)propanamido)-39,43-dioxo-2,5,8,11,14,17,20,23,26,29,32,35-dodecaoxa-38,44-diazaheptatetracontan-47-amido)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (1-11) trifluoroacetate

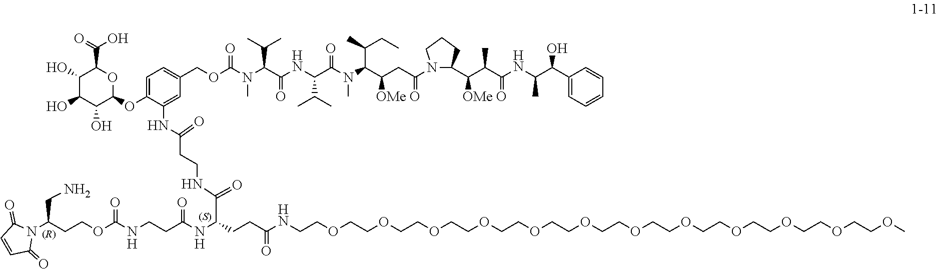

To the solution of 1-11a (50 mg, 0.023 mmol) in CH₂Cl₂ (2 mL) was added TFA (0.43 mL, 5.73 mmol) at 0° C., stirred at 0° C. for 30 min. The resulting solution was purified by prep-HPLC (Method: column XBridge Prep C18 OBD 5 μm 19*150 mm; Mobile phase: A-water (0.1% TFA): B-acetonitrile; Flow rate: 20 mL/min, the fraction was lyophilized to give 1-11 (trifluoroacetate) (36 mg, 71.5% yield) as a white solid. MS (ESI) m/z: 2083.0 [M+H]⁺.

### Example 1-12

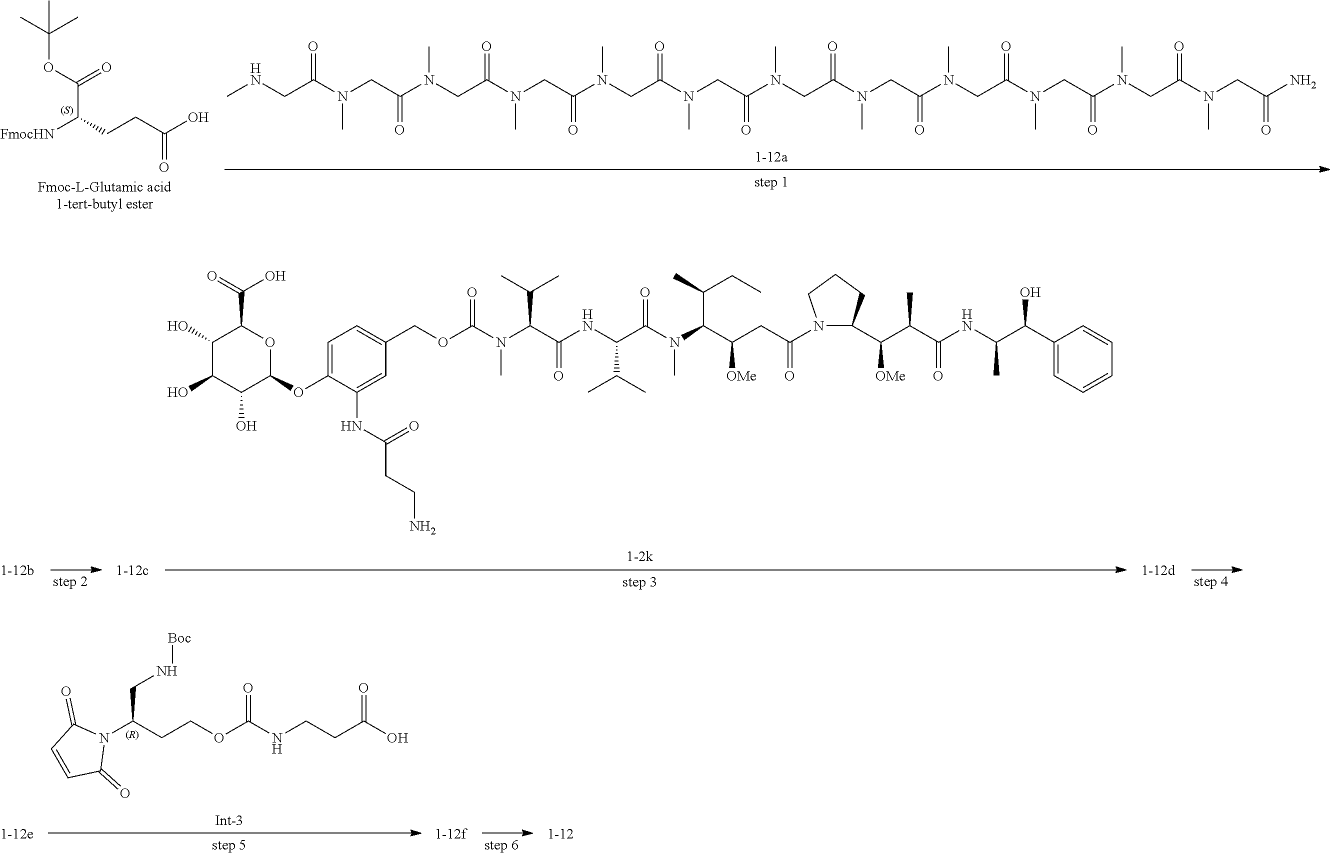

### Step 1: tert-butyl(S)-40-((((9H-fluoren-9-yl)methoxy)carbonyl)amino)-1-amino-3,6,9,12,15,18,21,24,27,30,33,36-dodecamethyl-1,4,7,10,13,16,19,22,25,28,31,34,37-tridecaoxo-3,6,9,12,15,18,21,24,27,30,33,36-dodecaazahentetracontan-41-oate (1-12b)

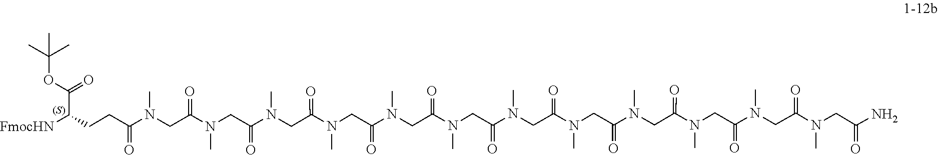

To the solution of 1-12a (45 mg, 0.052 mmol) and Fmoc-L-Glutamic acid 1-tert-butyl ester (23.3 mg, 0.054 mmol) in dry DMF (1 mL) were added HATU (23.8 mg, 0.062 mmol) and DIPEA (0.028 mL, 0.155 mmol), stirred at r.t. for 30 min. The resulting solution was purified by prep-HPLC (Method: column XBridge Prep C18 OBD 5 μm 19*150 mm; Mobile phase: A-water (0.1% FA): B-acetonitrile; Flow rate: 20 mL/min, the fraction was lyophilized to give 1-12b (36 mg, 71.5% yield) as a white solid. MS (ESI) m/z: 1278.3 [M+H]⁺.

### Step 2: (S)-40-((((9H-fluoren-9-yl)methoxy)carbonyl)amino)-1-amino-3,6,9,12,15,18,21,24,27,30,33,36-dodecamethyl-1,4,7,10,13,16,19,22,25,28,31,34,37-tridecaoxo-3,6,9,12,15,18,21,24,27,30,33,36-dodecaazahentetracontan-41-oic acid (1-12c)

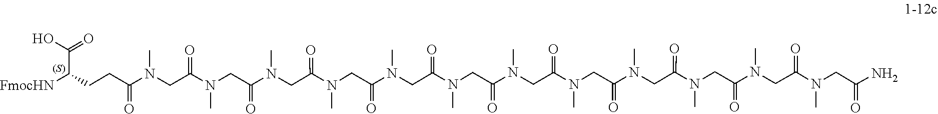

To the solution of 1-12b (45 mg, 0.035 mmol) in CH₂Cl₂ (0.4 mL) was added TFA (0.19 mL, 2.48 mmol), stirred at r.t. for 3 h. The resulting solution was concentrated under vacuo and co-evaporated with toluene five times to give 1-12c (40 mg, crude) as a colorless oil, which directly used in the next step. MS (ESI) m/z: 1222.0 [M+H]⁺.

### Step 3: (2S,3S,4S,5R,6S)-6-(2-((S)-40-((((9H-fluoren-9-yl)methoxy)carbonyl)amino)-1-amino-3,6,9,12,15,18,21,24,27,30,33,36-dodecamethyl-1,4,7,10,13,16,19,22,25,28,31,34,37,41-tetradecaoxo-3,6,9,12,15,18,21,24,27,30,33,36,42-tridecaazapentatetracontan-45-amido)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (1-12d)

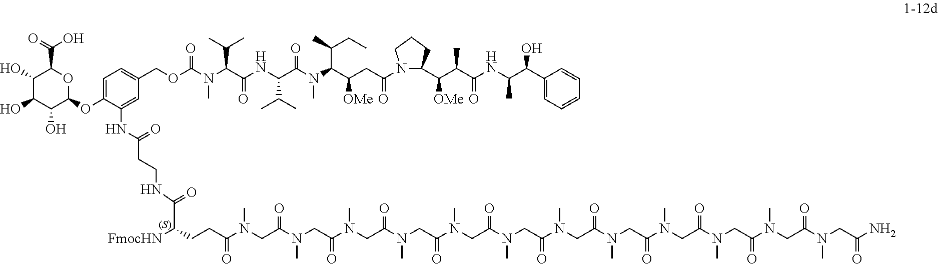

To the solution of 1-12c (40 mg, 0.032 mmol) in dry DMF (1 mL) were added HATU (15 mg, 0.039 mmol) and DIPEA (0.012 mL, 0.064 mmol), stirred at r.t. for 10 min. Then to the above mixture was added 1-2k (trifluoroacetate) (40 mg, 0.032 mmol), stirred at r.t. for 10 min. The resulting solution was purified by prep-HPLC (Method: column XBridge Prep C18 OBD 5 um 19*150 mm; Mobile phase: A-water (0.1% FA): B-acetonitrile; Flow rate: 20 mL/min, the fraction was lyophilized to give 1-12d (25 mg, 33.3% yield) as a white solid. MS (ESI) m/z: 2333.5 [M+H]⁺.

### Step 4: (2S,3S,4S,5R,6S)-6-(4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-((S)-1,40-diamino-3,6,9,12,15,18,21,24,27,30,33,36-dodecamethyl-1,4,7,10,13,16,19,22,25,28,31,34,37,41-tetradecaoxo-3,6,9,12,15,18,21,24,27,30,33,36,42-tridecaazapentatetracontan-45-amido)phenoxy)-3,4,5-trihydroxytetrahydro-2H-pyran-2-carboxylic acid (1-12e)

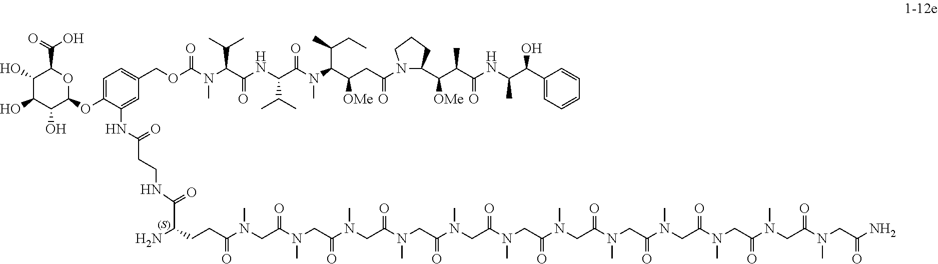

To the solution of 1-12d (25 mg, 0.011 mmol) in dry DMF (1 mL) was added Et₂NH (0.034 mL, 0.32 mmol), stirred at r.t. for 20 min. 1-12e (22 mg, crude) was co-evaporated with toluene five times to remove solvent, which was directly used in the next step. MS (ESI) m/z: 2111.5 [M+H]

### Step 5: (2S,3S,4S,5R,6S)-6-(2-((S)-1-amino-40-((R)-7-(2,5-dioxo-2,5-dihydro-1H-pyrrol-1-yl)-2,2-dimethyl-4,11-dioxo-3,10-dioxa-5,12-diazapentadecan-15-amido)-3,6,9,12,15,18,21,24,27,30,33,36-dodecamethyl-1,4,7,10,13,16,19,22,25,28,31,34,37,41-tetradecaoxo-3,6,9,12,15,18,21,24,27,30,33,36,42-tridecaazapentatetracontan-45-amido)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (1-12f)

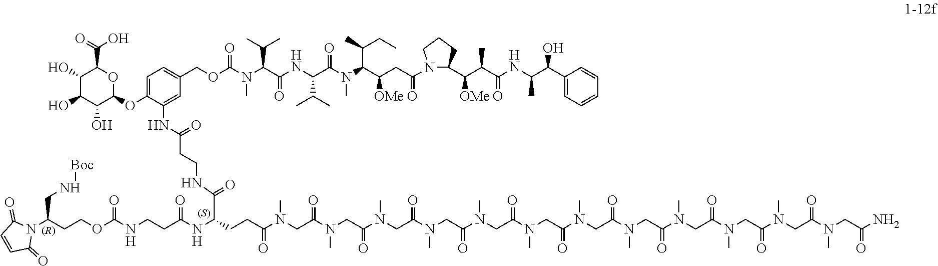

To the solution of Int-3 (5 mg, 0.013 mmol) in dry DMF (1 mL) were added HATU (4.8 mg, 0.013 mmol) and DIPEA (0.004 mL, 0.021 mmol), stirred at r.t. for 10 min. Then to the above mixture was added 1-12e (22 mg, 0.01 mmol), stirred at r.t. for 10 min. The resulting solution was purified by prep-HPLC (Method: column XBridge Prep C18 OBD 5 μm 19*150 mm; Mobile phase: A-water (0.1% FA): B-acetonitrile: Flow rate: 20 mL/min, the fraction was lyophilized to give 1-12f (13 mg, 50.1% yield) as a white solid. MS (ESI) m/z: 2491.8 [M+H]⁺.

### Step 6: (2S,3S,4S,5R,6S)-6-(2-((S)-1-amino-40-(3-((((R)-4-amino-3-(2,5-dioxo-2,5-dihydro-1H-pyrrol-1-yl) butoxy)carbonyl)amino)propanamido)-3,6,9,12,15,18,21,24,27,30,33,36-dodecamethyl-1,4,7,10,13,16,19,22,25,28,31,34,37,41-tetradecaoxo-3,6,9,12,15,18,21,24,27,30,33,36,42-tridecaazapentatetracontan-45-amido)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (1-12) trifluoroacetate

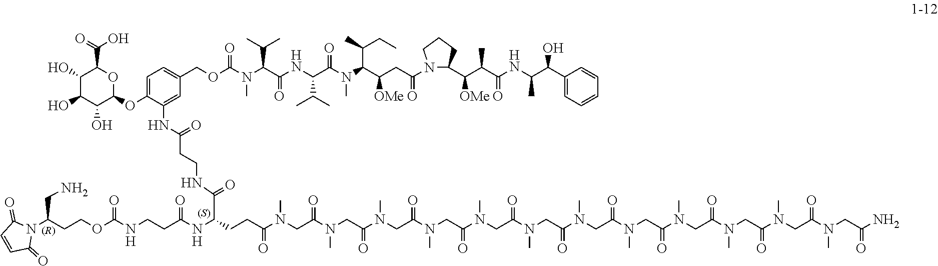

To the solution of 1-12f (13 mg, 0.005 mmol) in CH₂Cl₂ (0.5 mL) was added TFA (0.1 mL, 1.30 mmol), stirred at 0° C. for 30 min. The resulting solution was purified by prep-HPLC (Method: column XBridge Prep C18 OBD 5 μm 19*150 mm; Mobile phase: A-water (0.1% TFA): B-acetonitrile; Flow rate: 20 mL/min, the fraction was lyophilized to give 1-12 (trifluoroacetate) (10 mg, 76.5% yield) as a white solid. MS (ESI) m/z: 2392.3 [M+H]⁺.

### Example 1-13

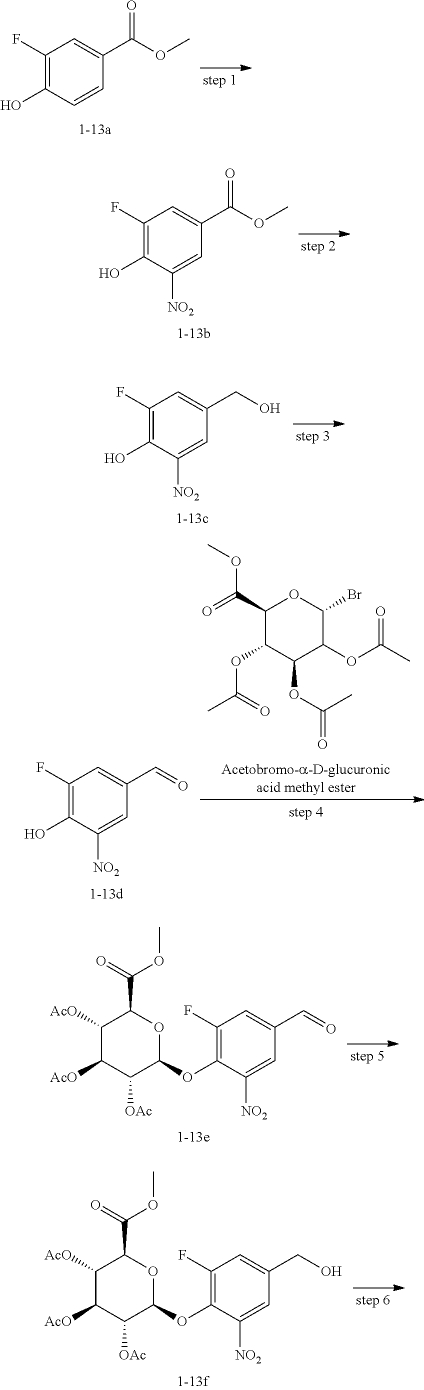

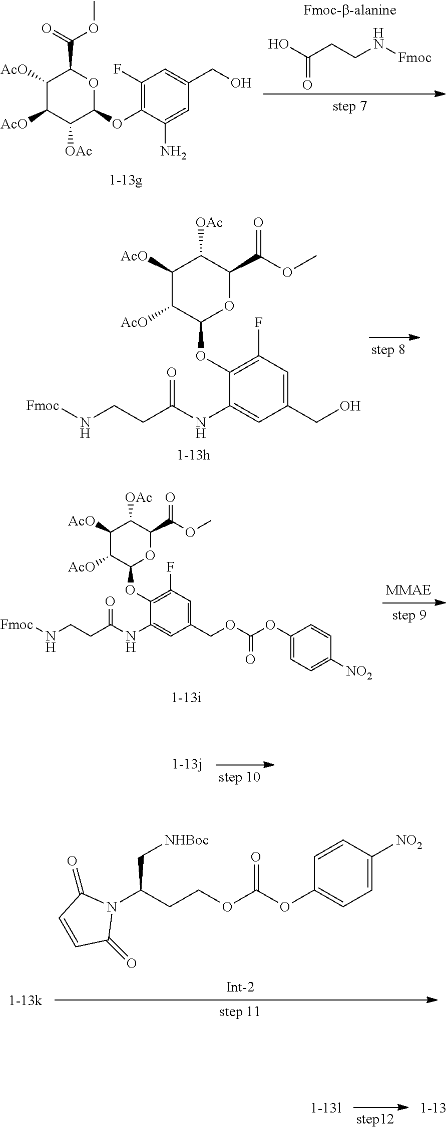

### Step 1: methyl 3-fluoro-4-hydroxy-5-nitrobenzoate (1-13b)

65% aq. nitric acid solution (1.4 mL, 30.43 mmol) and fuming nitric acid (2.1 mL, 21.43 mmol) were added at −10° C. to a solution of 1-13a (1.80 g, 10.6 mmol) in diethyl ether (50 mL). The ice bath was removed and the reaction mixture was stirred at 30° C. for 2 h, then partitioned between water and ethyl acetate. The organic layer was concentrated and purified by flash column chromatography (Petroleum ether/EtOAc=66/34) to give 1-13b as a yellow solid (600 mg, 26.3% yield). MS (ESI) m/z: 214.0 [M−H⁺]

### Step 2:2-fluoro-4-(hydroxymethyl)-6-nitrophenol (1-13c)

1-13b (1.20g, 5.58 mmol) was dissolved in dry CH₂Cl₂ (100 mL), Diisobutylaluminum hydride (1 N, 16 mL) was added at −78° C. under nitrogen atmosphere. The mixture was stirred at −78° C. for 1h then quenched by MeOH (4 mL) followed by 1N HCl (4 mL). The mixture was purified by flash column chromatography (Petroleum ether/EtOAc=50/50) to give 1-13c as a yellow solid (830 mg, 80.6% yield). MS (ESI) m/z: 186.1 [M−H⁺]

### Step 3:3-fluoro-4-hydroxy-5-nitrobenzaldehyde (1-13d)

To a solution of 1-13c (830 mg, 4.44 mmol) in dry CH₂Cl₂ (20 mL) was added Dess-Martin periodinane (2.82 g, 6.65 mmol). Then the mixture was moved to r.t. and stirred for 2 h. The mixture was quenched with aqueous Na₂S₂O₃ and Na₂CO₃ and purified by flash column chromatography (Petroleum ether/EtOAc=50/50) to give 1-13d as a yellow solid (300 mg, 36.5% yield). MS (ESI) m/z: 184.1 [M−H⁺]

### Step 4: (2S,3R,4S,5S,6S)-2-(2-fluoro-4-formyl-6-nitrophenoxy)-6-(methoxycarbonyl)tetrahydro-2H-pyran-3,4,5-triyl triacetate (1-13e)

To a solution of Acetobromo-α-D-glucuronic acid methyl ester (579 mg, 1.46 mmol) and Ag₂O (751 mg, 3.24 mmol) in CH₃CN (10 mL) was added 1-13d (300 mg, 1.62 mmol). The mixture was stirred at r.t. for 4h in the dark. The suspension was filtered through Celite, the filtrate was partitioned between ethyl acetate (50 mL) and water (50 mL). The organic layer was washed with aqueous NaHCO₃ (50 mL) three times, dried over Na₂SO₄, concentrated to give 1-13e as a yellow solid (648 mg, 79.8% yield) without further purification. MS (ESI) m/z: 524.4 [M+H]⁺

### Step 5: (2S,3R,4S,5S,6S)-2-(2-fluoro-4-(hydroxymethyl)-6-nitrophenoxy)-6-(methoxycarbonyl)tetrahydro-2H-pyran-3,4,5-triyl triacetate (1-13f)

To a solution of 1-13e (648 mg, 1.29 mmol) in IPA/CH₂Cl₂ ( 3/12 mL) was added NaBH₄ (48.90 mg, 1.29 mmol) at 0° C. The mixture was moved to r.t. and stirred for 1 h, then quenched with aqueous NH₄Cl. The mixture was partitioned between water and dichloromethane. The organic layer was collected, concentrated, and purified by flash column chromatography (Petroleum ether/EtOAc=50/50) to give 1-13d as a yellow solid (350 mg, 53.8% yield). MS (ESI) m/z: 526.5 [M+H]⁺

### Step 6: (2S,3R,4S,5S,6S)-2-(2-amino-6-fluoro-4-(hydroxymethyl)phenoxy)-6-(methoxycarbonyl)tetrahydro-2H-pyran-3,4,5-triyl triacetate (1-13g)

To a solution of 1-13f (350 mg, 0.70 mmol) in THE/MeOH/H₂O (5/1/1 mL) was added Fe (388 mg, 6.95 mmol) and NH₄Cl (186 mg, 3.48 mmol). The mixture was refluxed at 70° C. for 4 h. The mixture was partitioned between water and dichloromethane. The organic layer was collected, concentrated give crude 1-13g as a colorless oil (330 mg) without further purification.

### Step 7: (2S,3R,4S,5S,6S)-2-(2-(3-((((9H-fluoren-9-yl)methoxy)carbonyl)amino)propanamido)-6-fluoro-4-(hydroxymethyl)phenoxy)-6-(methoxycarbonyl)tetrahydro-2H-pyran-3,4,5-triyl triacetate (1-13h)

To a solution of 1-13g (330 mg, 0.70 mmol) and Fmoc-β-alanine (260 mg, 0.84 mmol) in CH₂Cl₂/DMF (5/1 mL) was added EEDQ (345 mg, 1.39 mmol). The mixture was stirred at r.t. overnight and purified by flash column chromatography (MeOH/CH₂Cl₂=5/95) to give 1-13d as a white solid (200 mg, 37.4% yield in two steps). MS (ESI) m/z: 767.6 [M+H]⁺

### Step 8: (2S,3R,4S,5S,6S)-2-(2-(3-((((9H-fluoren-9-yl)methoxy)carbonyl)amino)propanamido)-6-fluoro-4-((((4-nitrophenoxy)carbonyl)oxy)methyl)phenoxy)-6-(methoxycarbonyl)tetrahydro-2H-pyran-3,4,5-triyl triacetate (1-13i)

To a solution of 1-13h (200 mg, 0.26 mmol) and 4-Nitrophenyl chloroformate (105 mg, 0.52 mmol) in dry CH₂Cl₂ (4 mL) was added DIPEA (136 μL, 0.78 mmol). The mixture was stirred at r.t. overnight and purified by flash column chromatography (MeOH/CH₂Cl₂=5/95) to give 1-13i as a white solid (100 mg, 41.2% yield). MS (ESI) m/z: 932.6 [M+H]⁺

### Step 9: (2S,3R,4S,5S,6S)-2-(2-(3-((((9H-fluoren-9-yl)methoxy)carbonyl)amino)propanamido)-4-((5S,8S,11R,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-6-fluorophenoxy)-6-(methoxycarbonyl)tetrahydro-2H-pyran-3,4,5-triyl triacetate (1-13j)

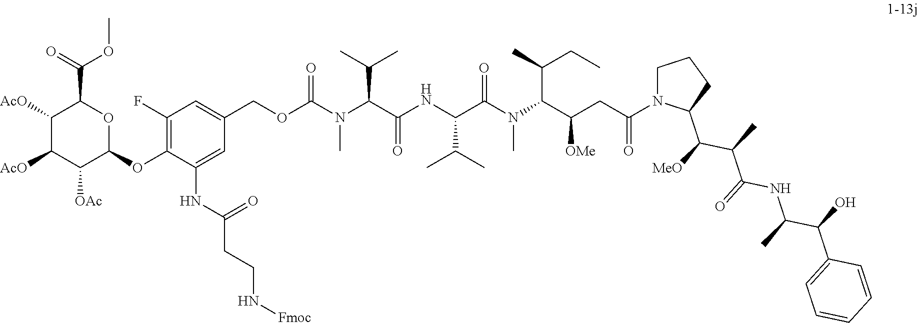

To a solution of 1-13i (100 mg, 0.11 mmol) in 2 mL DMF was added MMAE (77 mg, 0.11 mmol) and DIPEA (56.1 μL, 0.32 mmol), followed by addition of HOBt (4.4 mg, 0.027 mmol). The mixture was stirred at r.t. for 24 h and partitioned between water and ethyl acetate. The organic layer was purified by flash column chromatography (MeOH/CH₂Cl₂=5/95) to obtain 1-13j as a white solid (101 mg, 62.3% yield). MS (ESI) m/z: 1511.5 [M+H]⁺

### Step 10: (2S,3S,4S,5R,6S)-6-(2-(3-aminopropanamido)-4-((5S,8S,11R,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-6-fluorophenoxy)-3,4,5-trihydroxytetrahydro-2H-pyran-2-carboxylic acid (1-13k)

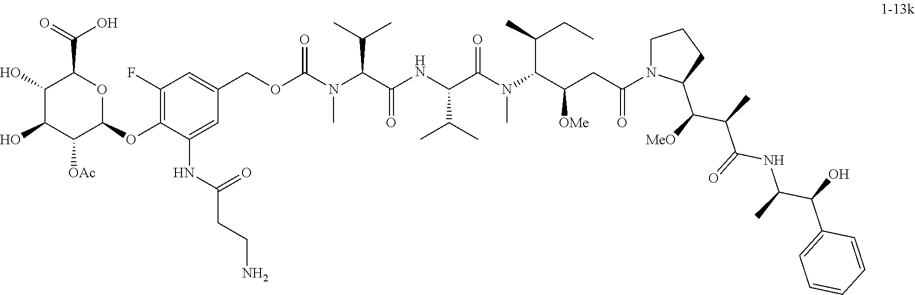 [SMILES: CC[C@H](C)[C@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1cc(F)c(O[C@@H]2O[C@H](C(=O)O)[C@@H](O)[C@H](O)[C@H]2OC(C)=O)c(NC(=O)CCN)c1)C(C)C]

To a solution of 1-13j (101 mg, 0.07 mmol) in THE/MeOH (1/1 mL) was added 1 N LiOH (400 μL) at 0° C. The mixture was stirred at r.t. for 20 min, quenched with HOAc (12 μL) and purified by prep-HPLC (Column: Xbridge Prep C18 OBD™ 5 μm, 19*150 mm; Mobile phase A: 0.1% FA in water, B: MeCN; Gradient: 30%-55% B; Flow rate: 20 mL/min), lyophilized to give 1-13k (40 mg, 52.1% yield). MS (ESI) m/z: 1149.2 [M+H]⁺

### Step 11: (2S,3S,4S,5R,6S)-6-(4-((5S,8S,11R,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-((R)-7-(2,5-dioxo-2,5-dihydro-1H-pyrrol-1-yl)-2,2-dimethyl-4,11-dioxo-3,10-dioxa-5,12-diazapentadecan-15-amido)-6-fluorophenoxy)-3,4,5-trihydroxytetrahydro-2H-pyran-2-carboxylic acid (1-13l)

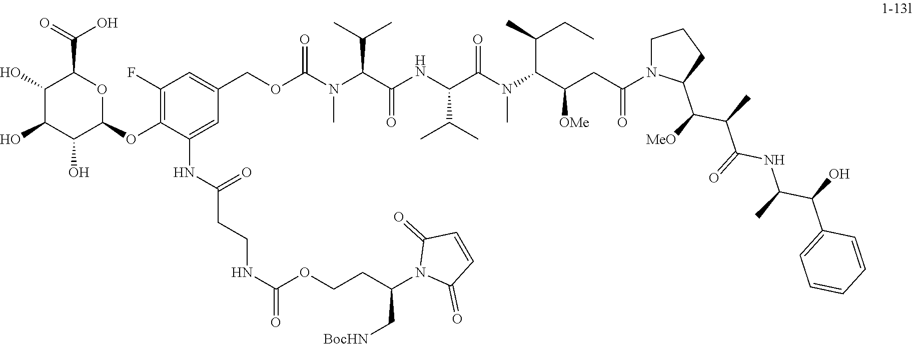 [SMILES: CC[C@H](C)[C@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1cc(F)c(O[C@@H]2O[C@H](C(=O)O)[C@@H](O)[C@H](O)[C@H]2O)c(NC(=O)CCNC(=O)OCC[C@H](CNC(=O)OC(C)(C)C)N2C(=O)C=CC2=O)c1)C(C)C]

To a solution of 1-13k (26.2 mg, 0.023 mmol) and Int-2 (12.3 mg, 0.027 mmol) in DMF (1 mL) was added DIPEA (12 μL, 0.069 mmol). The mixture was stirred at r.t. overnight and purified by prep-HPLC (Column: Xbridge Prep C18 OBD™ 5 μm, 19*150 mm; Mobile phase A: 0.1% FA in water, B: MeCN; Gradient: 45%-85% B; Flow rate: 20 mL/min), lyophilized to give 1-13l (15 mg, 45.1% yield). MS (ESI) m/z: 1459.6 [M+H]⁺

### Step 12: (2S,3S,4S,5R,6S)-6-(2-(3-((((R)-4-amino-3-(2,5-dioxo-2,5-dihydro-1H-pyrrol-1-yl) butoxy)carbonyl)amino)propanamido)-4-((5S,8S,11R,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-6-fluorophenoxy)-3,4,5-trihydroxytetrahydro-2H-pyran-2-carboxylic acid (1-13)

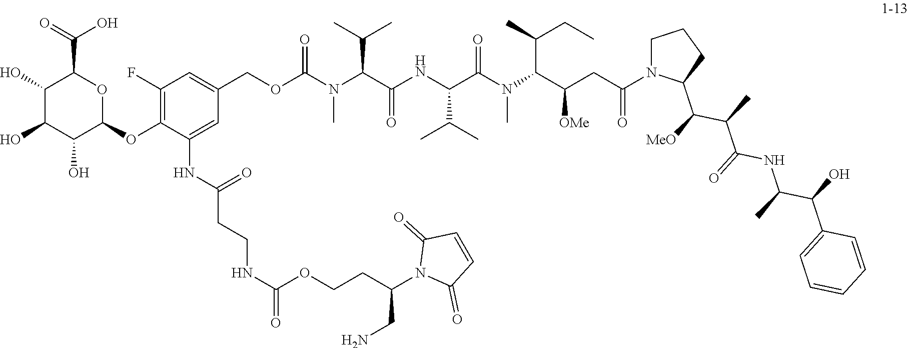 [SMILES: CC[C@H](C)[C@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1cc(F)c(O[C@@H]2O[C@H](C(=O)O)[C@@H](O)[C@H](O)[C@H]2O)c(NC(=O)CCNC(=O)OCC[C@H](CN)N2C(=O)C=CC2=O)c1)C(C)C]

1-13l (15 mg, 10.28 μmol) was dissolved in CH₂Cl₂/TFA (1/0.2 mL) and stirred at r.t. for 30 min. The mixture purified by prep-HPLC (Column: Xbridge Prep C18 OBD™ 5 μm, 19*150 mm; Mobile phase A: 0.1% TFA in water, B: MeCN; Gradient: 40%-70% B; Flow rate: 20 mL/min), lyophilized to give 1-13 (5 mg, 35.8% yield). MS (ESI) m/z: 1359.3 [M+H]⁺

### Example 1-14

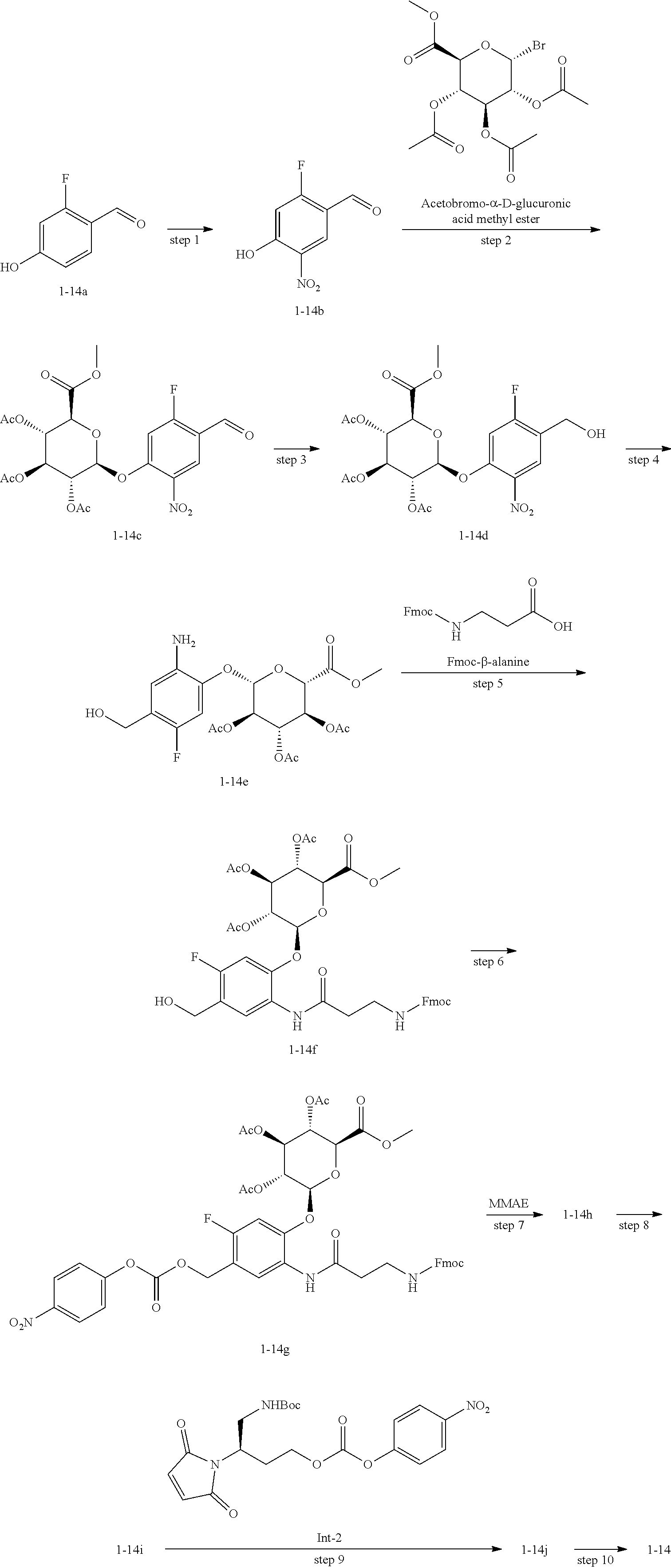

### Step 1:2-fluoro-4-hydroxy-5-nitrobenzaldehyde (1-14b)

1-14a (1.50 g, 10.71 mmol) was dissolved in sulfuric acid (12.5 mL). 65% nitric acid solution (690 μL, 10.71 mmol) was dissolved in sulfuric acid (2.5 mL) at −20° C. and added to the reaction solution. The reaction mixture was stirred at −20° C. for 2 h, then quenched with ice water (100 mL), extracted with EtOAc (100 mL*2). The combined organics were dried over Na₂SO₄, concentrated, and purified by flash column chromatography to give 1-14b as a yellow solid (1.59 g, 80.2% yield). MS (ESI) m/z: 184.0 [M−H⁺]

### Step 2: (2S,3R,4S,5S,6S)-2-(5-fluoro-4-formyl-2-nitrophenoxy)-6-(methoxycarbonyl)tetrahydro-2H-pyran-3,4,5-triyl triacetate (1-14c)

To a solution of Acetobromo-α-D-glucuronic acid methyl ester (3.07 g, 7.73 mmol) and Ag₂O (2.99 g, 12.88 mmol) in CH₃CN (50 mL) was added 1-14b (1.59 g, 8.59 mmol). The mixture was stirred at r.t. for 4h in the dark. The suspension was filtered through Celite, the filtrate was partitioned between ethyl acetate (100 mL) and water (100 mL). The organic layer was washed with aqueous Na₂CO₃ (100 mL) three times, dried over Na₂SO₄, concentrated to give 1-14c as a yellow solid (3.64 g, 84.5% yield) without further purification. MS (ESI) m/z: 524.5 [M+Na]⁺

### Step 3: (2S,3R,4S,5S,6S)-2-(5-fluoro-4-(hydroxymethyl)-2-nitrophenoxy)-6-(methoxycarbonyl)tetrahydro-2H-pyran-3,4,5-triyl triacetate (1-14d)

To a solution of 1-14c (3.63 g, 7.24 mmol) in IPA/CH₂Cl₂ (10/40 mL) was added NaBH₄ (0.27 g, 7.24 mmol) at 0° C. The mixture was moved to r.t. and stirred for 3 h, then quenched with aqueous NH₄Cl. The mixture was partitioned between water and dichloromethane. The organic layer was collected, concentrated, and purified by flash column chromatography (Petroleum ether/EtOAc=50/50) to give 1-14d as a yellow solid (1.88 g, 51.6% yield). MS (ESI) m/z: 526.5 [M+Na]⁺

### Step 4: (2S,3R,4S,5S,6S)-2-(2-amino-5-fluoro-4-(hydroxymethyl)phenoxy)-6-(methoxycarbonyl)tetrahydro-2H-pyran-3,4,5-triyl triacetate (1-14e)

To a solution of 1-14d (980 mg, 1.95 mmol) in THF/MeOH/H₂O (5/1/1 mL) was added Fe (1.09 g, 19.47 mmol) and NH₄Cl (521 mg, 9.73 mmol). The mixture was refluxed at 70° C. for 4 h. The mixture was partitioned between water and dichloromethane. The organic layer was collected, concentrated give 1-14e as a colorless oil (880 mg, 95.5% yield). MS (ESI) m/z: 474.5 [M+H]⁺.

### Step 5: (2S,3R,4S,5S,6S)-2-(2-(3-((((9H-fluoren-9-yl)methoxy)carbonyl)amino)propanamido)-5-fluoro-4-(hydroxymethyl)phenoxy)-6-(methoxycarbonyl)tetrahydro-2H-pyran-3,4,5-triyl triacetate (1-14f)

To a solution of 1-14e (880 mg, 1.86 mmol) and Fmoc-β-alanine (694 mg, 2.23 mmol) in CH₂Cl₂/DMF (10/1 mL) was added EEDQ (919 mg, 3.72 mmol). The mixture was stirred at r.t. overnight and purified by flash column chromatography (MeOH/CH₂Cl₂=5/95) to give 1-14f as a white solid (1.06 g, 74.4% yield). MS (ESI) m/z: 767.6 [M+H]⁺.

### Step 6: (2S,3R,4S,5S,6S)-2-(2-(3-((((9H-fluoren-9-yl)methoxy)carbonyl)amino)propanamido)-5-fluoro-4-((((4-nitrophenoxy)carbonyl)oxy)methyl)phenoxy)-6-(methoxycarbonyl)tetrahydro-2H-pyran-3,4,5-triyl triacetate (1-14g)

To a solution of 1-14f (190 mg, 0.25 mmol) and 4-Nitrophenyl chloroformate (80 mg, 0.40 mmol) in dry CH₂Cl₂ (4 mL) was added DIPEA (90 μL, 0.50 mmol). The mixture was stirred at r.t. for 6 h and purified by flash column chromatography (MeOH/CH₂Cl₂=5/95) to give 1-14g as a white solid (190 mg, 82.3% yield). MS (ESI) m/z: 932.6 [M+H]⁺.

### Step 7: (2S,3R,4S,5S,6S)-2-(2-(3-((((9H-fluoren-9-yl)methoxy)carbonyl)amino)propanamido)-4-((5S,8S,11R,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-5-fluorophenoxy)-6-(methoxycarbonyl)tetrahydro-2H-pyran-3,4,5-triyl triacetate (1-14h)

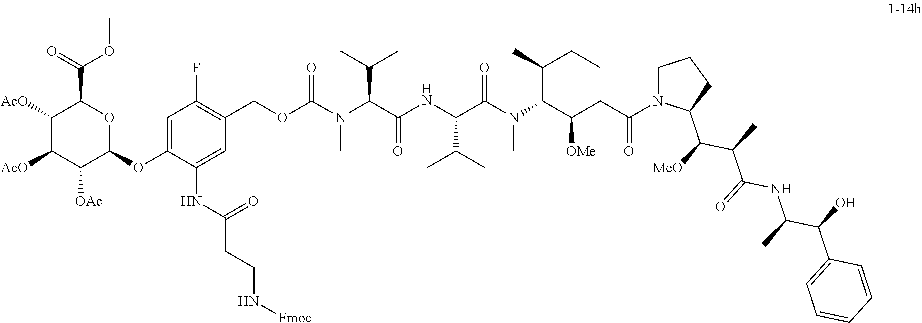

To a solution of 1-14g (190 mg, 0.20 mmol) in 2 mL DMF was added MMAE (146 mg, 0.20 mmol) and DIPEA (106 μL, 0.61 mmol), followed by addition of HOBt (6.9 mg, 0.069 mmol). The mixture was stirred at r.t. for 48 h and partitioned between water and ethyl acetate. The organic layer was purified by flash column chromatography (MeOH/CH₂Cl₂=5/95) to obtain crude 1-14h as a yellow solid (40 mg). MS (ESI) m/z: 1512.2 [M+H]⁺.

### Step 8: (2S,3S,4S,5R,6S)-6-(2-(3-aminopropanamido)-4-((5S,8S,11R,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-6-fluorophenoxy)-3,4,5-trihydroxytetrahydro-2H-pyran-2-carboxylic acid (1-14i)

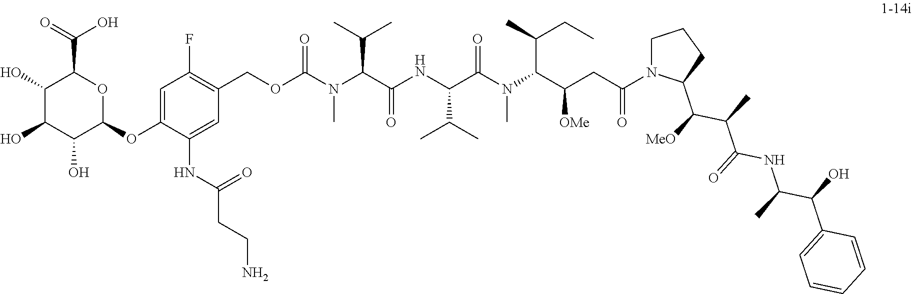 [SMILES: CC[C@H](C)[C@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1cc(NC(=O)CCN)c(O[C@@H]2O[C@H](C(=O)O)[C@@H](O)[C@H](O)[C@H]2O)cc1F)C(C)C]

To a solution of crude 1-14h (40 mg, 0.026 mmol) in MeOH (1 mL) was added 1 N LiOH (400 μL) at 0° C. The mixture was stirred at r.t. for 20 min, quenched with HOAc (12 μL). The mixture was concentrated and then dissolved in DMF (1 mL). Et₂NH (50 μL, 0.48 mmol) was added and the mixture was stirred at r.t. for 30 min. The mixture was concentrated and purified by prep-HPLC (Column: Sunfire Prep C18 OBD™ 5 μm, 19*150 mm; Mobile phase A: 0.1% FA in water, B: MeCN; Gradient: 30%-60% B; Flow rate: 20 mL/min), lyophilized to give 1-14i (20 mg, 8.5% yield in two steps). MS (ESI) m/z: 1149.1 [M+H]⁺.

### Step 9: (2S,3S,4S,5R,6S)-6-(4-((5S,8S,11R,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-((R)-7-(2,5-dioxo-2,5-dihydro-1H-pyrrol-1-yl)-2,2-dimethyl-4,11-dioxo-3,10-dioxa-5,12-diazapentadecan-15-amido)-5-fluorophenoxy)-3,4,5-trihydroxytetrahydro-2H-pyran-2-carboxylic acid (1-14j)

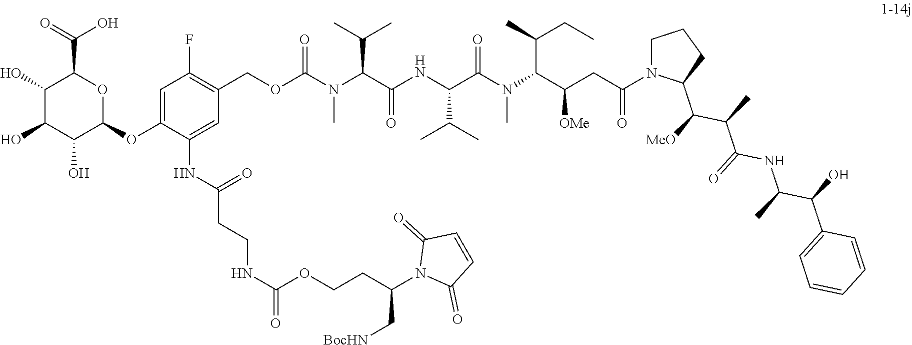 [SMILES: CC[C@H](C)[C@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1cc(NC(=O)CCNC(=O)OCC[C@H](CNC(=O)OC(C)(C)C)N2C(=O)C=CC2=O)c(O[C@@H]2O[C@H](C(=O)O)[C@@H](O)[C@H](O)[C@H]2O)cc1F)C(C)C]

To a solution of 1-14i (20 mg, 0.017 mmol) and Int-2 (11.74 mg, 0.026 mmol) in DMF (1 mL) was added DIPEA (9.1 μL, 0.052 mmol). The mixture was stirred at r.t. for 3 h and purified by prep-HPLC (Column: Sunfire Prep C18 OBD™ 5 μm, 19*150 mm; Mobile phase A: 0.1% FA in water, B: MeCN; Gradient: 30%-80% B; Flow rate: 20 mL/min), lyophilized to give 1-14j (13 mg, 51.2% yield). MS (ESI) m/z: 1459.3 [M+H]⁺.

### Step 10: (2S,3S,4S,5R,6S)-6-(2-(3-((((R)-4-amino-3-(2,5-dioxo-2,5-dihydro-1H-pyrrol-1-yl) butoxy)carbonyl)amino)propanamido)-4-((5S,8S,11R,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-5-fluorophenoxy)-3,4,5-trihydroxytetrahydro-2H-pyran-2-carboxylic acid (1-14)

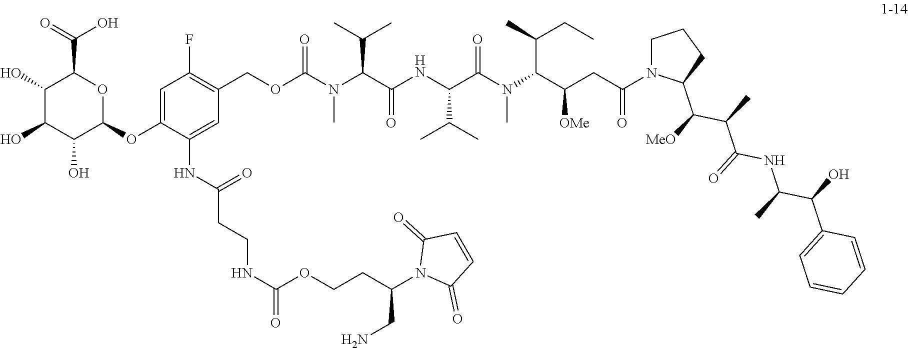 [SMILES: CC[C@H](C)[C@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1cc(NC(=O)CCNC(=O)OCC[C@H](CN)N2C(=O)C=CC2=O)c(O[C@@H]2O[C@H](C(=O)O)[C@@H](O)[C@H](O)[C@H]2O)cc1F)C(C)C]

1-14j (13 mg, 8.92 μmol) was dissolved in CH₂Ch/TFA (1/0.2 mL) and stirred at r.t. for 30 min. The mixture purified by prep-HPLC (Column: Xbridge Prep C18 OBD™ 5 μm, 19*150 mm; Mobile phase A: 0.1% TFA in water, B: MeCN; Gradient: 35%-60% B; Flow rate: 20 mL/min), lyophilized to give 1-14 (4.3 mg, 35.5% yield). MS (ESI) m/z: 1359.1 [M+H]⁺.

### Example 1-15

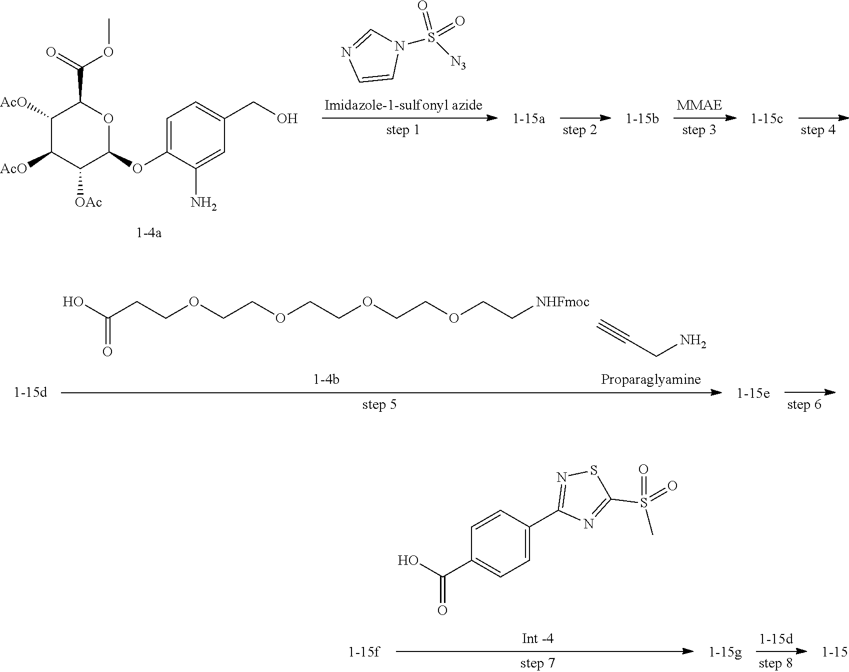

### Step 1 (2S,3R,4S,5S,6S)-2-(2-azido-4-(hydroxymethyl)phenoxy)-6-(methoxycarbonyl)tetrahydro-2H-pyran-3,4,5-triyl triacetate (1-15a)

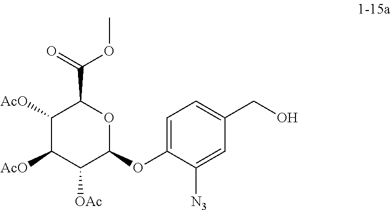 [SMILES: COC(=O)[C@H]1O[C@@H](Oc2ccc(CO)cc2N=[N+]=[N-])[C@H](OC(C)=O)[C@@H](OC(C)=O)[C@@H]1OC(C)=O]

1-4a (450 mg, 0.99 mmol) was dissolved in dry THF (2 mL). The mixture was cooled to 0° C., then Imidazole-1-sulfonyl azide (414.2 mg, 1.98 mmol) in 1 mL of MeOH was added, followed by addition of K₂CO₃ in 1 mL of H₂O and cat. amount of CuSO₄. The mixture was stirred at r.t. for 2 h under a nitrogen atmosphere. EtOAc (5 mL) was added, the organic phase was collected, dried over Na₂SO₄, concentrated, and then purified by column chromatography (Petroleum ether/EtOAc=50/50) to obtain 2-15a as a white solid (350 mg, 87.2%). MS (ESI) m/z: 504.2 [M+Na]⁺. ¹H NMR (400 MHz, CDCl₃) δ 7.17-7.03 (m, 3H), 5.44-5.26 (m, 3H), 5.07 (d, J=7.1 Hz, 1H), 4.64 (d, J=2.2 Hz, 2H), 4.13 (dd, J=6.8, 2.8 Hz, 1H), 3.83-3.71 (m, 3H), 2.17-2.00 (m, 9H).

### Step 2 (2S,3R,4S,5S,6S)-2-(2-azido-4-((((4-nitrophenoxy)carbonyl)oxy)methyl)phenoxy)-6-(methoxycarbonyl)tetrahydro-2H-pyran-3,4,5-triyl triacetate (1-15b)

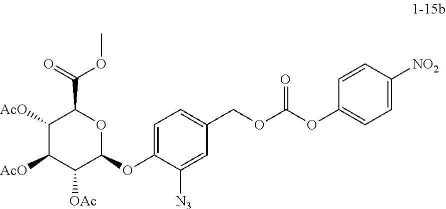 [SMILES: COC(=O)[C@H]1O[C@@H](Oc2ccc(COC(=O)Oc3ccc([N+](=O)[O-])cc3)cc2N=[N+]=[N-])[C@H](OC(C)=O)[C@@H](OC(C)=O)[C@@H]1OC(C)=O]

1-15a (100 mg, 0.21 mmol) was dissolved in dry DMF (0.5 mL). The mixture was cooled to 0° C., 4-Nitrophenyl chloroformate (50.24 mg, 0.25 mmol) was added, followed by addition of DIPEA (79.58 μL, 0.42 mmol). The mixture was stirred at r.t. for 6 h under a nitrogen atmosphere. The reaction solution was diluted with EtOAc (5 mL), washed with H₂O (5 mL). The organic phase was collected, dried over Na₂SO₄, concentrated, and then purified by column chromatography (Petroleum ether/EtOAc=50/50) to obtain 1-15b as a light-yellow solid (70 mg, 52.2%). MS (ESI) m/z: 668.5 [M+Na]⁺

### Step 3 (2S,3R,4S,5S,6S)-2-(2-azido-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-6-(methoxycarbonyl)tetrahydro-2H-pyran-3,4,5-triyl triacetate (1-15c)

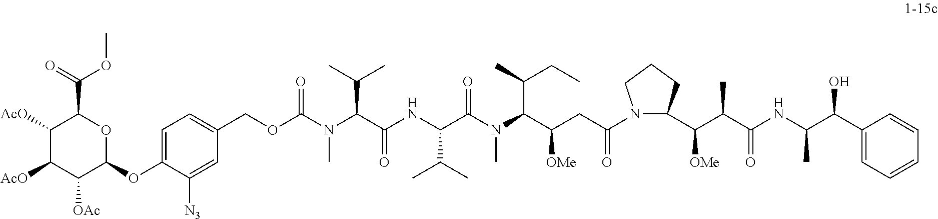 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2O[C@H](C(=O)OC)[C@@H](OC(C)=O)[C@H](OC(C)=O)[C@H]2OC(C)=O)c(N=[N+]=[N-])c1)C(C)C]

To a solution of 1-15b (70 mg, 0.11 mmol) in 1 mL DMF was added MMAE (85.51 mg, 0.12 mmol) and DIPEA (59.1 μL, 0.33 mmol), followed by addition of HOBt (4.4 mg, 0.032 mmol). The mixture was stirred at r.t. for 12 h under a nitrogen atmosphere. The solvent was then removed by evaporation, and the crude product was purified by column chromatography (MeOH/CH₂Cl₂=5/95) to obtain 1-15c as a white solid (65 mg, 50%). MS (ESI) m/z: 1227.1 [M+H]⁺.

### Step 4 (2S,3S,4S,5R,6S)-6-(2-azido-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (1-15d)

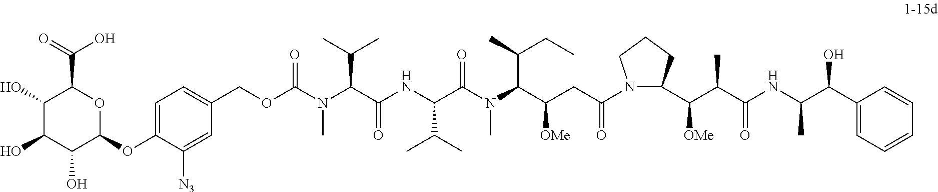 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2O[C@H](C(=O)O)[C@@H](O)[C@H](O)[C@H]2O)c(N=[N+]=[N-])c1)C(C)C]

To a solution of 1-15c (65 mg, 0.05 mmol) in 2 mL THF was added 0.5 M LiOH solution (1 mL) at 0° C. The mixture was stirred at 0° C. for 15 min under a nitrogen atmosphere. The reaction was quenched with AcOH, and then purified by prep-HPLC (Method: column: XBridge Prep C18 OBD 5 μm 19*1250 mm; Mobile phase: A-water (0.1% formic acid): B-acetonitrile: Flow rate: 20 mL/min) to provide 1-15d as a white solid after lyophilization (40 mg, 69.5% yield). MS (ESI) m/z: 1086.4 [M+H]⁺.

### Step 5: (9H-fluoren-9-yl)methyl (15-oxo-3,6,9,12-tetraoxa-16-azanonadec-18-yn-1-yl)carbamate (1-15e)

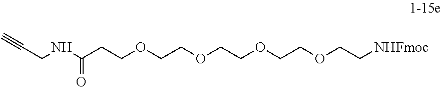

To a solution of 1-4b (200 mg, 0.41 mmol) in 2 mL DMF was added HATU (312 mg, 0.82 mmol) and DIPEA (224 μL, 1.23 mmol) at 0° C. The mixture was stirred at 0° C. for 10 min. Propargylamine (25 μL, 0.45 mmol) was added. The mixture was stirred at r.t. for 1 h under a nitrogen atmosphere. The solvent was then removed by evaporation, and the crude product was purified by column chromatography (MeOH/CH₂Cl₂=5/95) to obtain 1-15e as a brown oil (200 mg, 93% yield). MS (ESI) m/z: 525.8 [M+H]⁺.

### Step 6:1-amino-N-(prop-2-yn-1-yl)-3,6,9,12-tetraoxapentadecan-15-amide (1-15f)

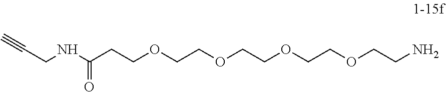 [SMILES: C#CCNC(=O)CCOCCOCCOCCOCCN]

To a solution of 1-15e (500 mg, 0.95 mmol) in 2 mL DMF was added diethylamine (0.98 mL, 9.5 mmol) at 0° C. The mixture was stirred at 0° C. for 30 min. The solvent was then removed by evaporation, and the crude product was purified by column chromatography (MeOH/CH₂Cl₂=5/95) to obtain 1-15f as a light-yellow solid (250 mg, 87% yield). MS (ESI) m/z: 303.3 [M+H]⁺.

### Step 7:1-(4-(5-(methylsulfonyl)-1,2,4-thiadiazol-3-yl)benzamido)-N-(prop-2-yn-1-yl)-3,6,9,12-tetraoxapentadecan-15-amide (1-15g)

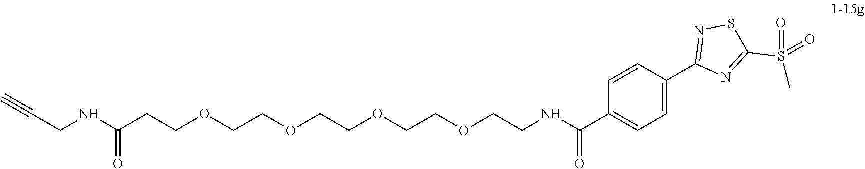 [SMILES: C#CCNC(=O)CCOCCOCCOCCOCCNC(=O)c1ccc(-c2nsc(S(C)(=O)=O)n2)cc1]

To a solution of 1-15f (10 mg, 0.033 mmol) in 0.5 mL DMF was added HATU (25 mg, 0.066 mmol) and DIPEA (18 μL, 0.099 mmol) at 0° C. The mixture was stirred at 0° C. for 10 min. Int-4 (10.34 mg, 0.036 mmol) was added. The mixture was stirred at r.t. for 1 h under a nitrogen atmosphere. The solvent was then removed by evaporation, and the crude product was purified by column chromatography (MeOH/CH₂Cl₂=5/95) to obtain 1-15g as a white solid (15 mg, 80%). MS (ESI) m/z: 569.6 [M+H]⁺.

### Step 8 (2S,3S,4S,5R,6S)-6-(4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-(4-(1-(4-(5-(methylsulfonyl)-1,2,4-thiadiazol-3-yl)phenyl)-1,17-dioxo-5,8,11,14-tetraoxa-2,18-diazanonadecan-19-yl)-1H-1,2,3-triazol-1-yl)phenoxy)-3,4,5-trihydroxytetrahydro-2H-pyran-2-carboxylic acid (1-15)

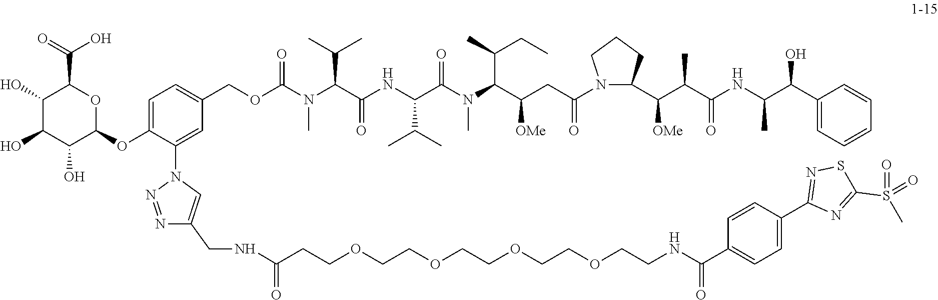 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2O[C@H](C(=O)O)[C@@H](O)[C@H](O)[C@H]2O)c(-n2cc(CNC(=O)CCOCCOCCOCCOCCNC(=O)c3ccc(-c4nsc(S(C)(=O)=O)n4)cc3)nn2)c1)C(C)C]

To a solution of 1-15d (20 mg, 0.018 mmol) and 1-15g (12.6 mg, 0.022 mmol) in 0.5 mL DMF was added DIPEA (6.7 μL, 0.037 mmol) at 0° C., followed by addition of cat. amount of CuI. The mixture was degassed with N₂ for 10 min and then stirred under a nitrogen atmosphere. The reaction was quenched with AcOH, and then purified by prep-HPLC (Method: column: XBridge Prep C18 OBD 5 μm 19*1250 mm; Mobile phase: A-water (0.1% formic acid): B-acetonitrile; Flow rate: 20 mL/min) to provide 1-15 as a white solid after lyophilization (7 mg, 23% yield). MS (ESI) m/z: 1654.9 [M+H]⁺.

### Example 1-16

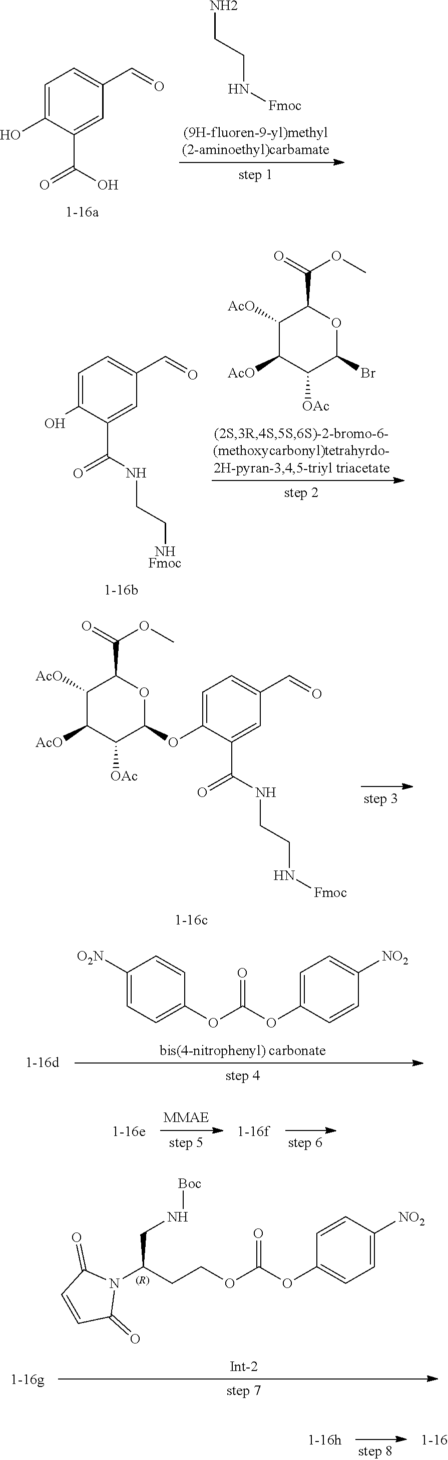

### Step 1: (9H-fluoren-9-yl)methyl (2-(5-formyl-2-hydroxybenzamido)ethyl)carbamate (1-16b)

To the solution of 1-16a (235 mg, 1.42 mmol) in dry DMF (10.0 mL) were added HATU (652 mg, 1.70 mmol) and DIPEA (0.50 mL, 2.83 mmol), stirred at r.t. for 10 min. To the above mixture was added (9H-fluoren-9-yl)methyl (2-aminoethyl)carbamate (501 mg, 1.56 mmol), stirred at r.t. for 20 min. The resulting solution was purified by prep-HPLC (Method: column XBridge Prep C18 OBD 5 um 19*150 mm; Mobile phase: A-water (0.1% FA): B-acetonitrile: Flow rate: 20 mL/min, the fraction was lyophilized to give 1-16b (220 mg, 36.1% yield) as a white solid. MS (ESI) m/z: 431.4 [M+H]⁺.

### Step 2: (2S,3R,4S,5S,6S)-2-(2-((2-((((9H-fluoren-9-yl)methoxy)carbonyl)amino)ethyl)carbamoyl)-4-formylphenoxy)-6-(methoxycarbonyl)tetrahydro-2H-pyran-3,4,5-triyl triacetate (1-16c)

To the solution of 1-16b (210 mg, 0.49 mmol) in dry MeCN (4 mL) were added (2S,3R,4S,5S,6S)-2-bromo-6-(methoxycarbonyl)tetrahydro-2H-pyran-3,4,5-triyl triacetate (215 mg, 0.54 mmol), 4 Å MS (500 mg) and Ag₂O (232, 1.95 mmol) under N₂ atmosphere, stirred at r.t. for 24 h. The resulting solution was filtrated and washed with MeCN (30 mL). The filtrate was concentrated and purified by silica column gel chromatography (EtOAc/petroleum ether=60/40) to give 1-16c (210 mg, 57.6% yield) as a white solid. MS (ESI) m/z: 747.6 [M+H]⁺.

### Step 3: (2S,3R,4S,5S,6S)-2-(2-((2-((((9H-fluoren-9-yl)methoxy)carbonyl)amino)ethyl)carbamoyl)-4-(hydroxymethyl)phenoxy)-6-(methoxycarbonyl)tetrahydro-2H-pyran-3,4,5-triyl triacetate (1-16d)

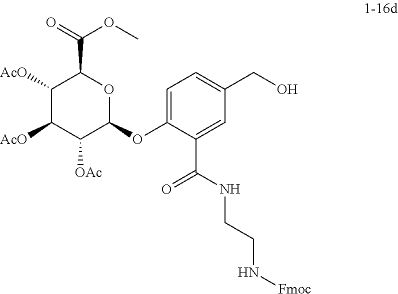

To the solution of 1-16c (210 mg, 0.28 mmol) in CH₂Cl₂ (4 mL) and isopropyl alcohol (1 mL) was added sodium borohydride (16 mg, 0.42 mmol) at 0° C., stirred at r.t. for 1 h. The resulting solution was quenched with sat. NH₄Cl and extracted with EtOAc (30 mL*3). The organic phase was concentrated and purified by silica gel column chromatography (CH₂Cl/MeOH=95/5) to give 1-16d (150 mg, 71.2% yield) as a colorless oil. MS (ESI) m/z: 749.4 [M+H]⁺.

### Step 4: (2S,3R,4S,5S,6S)-2-(2-((2-((((9H-fluoren-9-yl)methoxy)carbonyl)amino)ethyl)carbamoyl)-4-((((4-nitrophenoxy)carbonyl)oxy)methyl)phenoxy)-6-(methoxycarbonyl)tetrahydro-2H-pyran-3,4,5-triyl triacetate (1-16e)

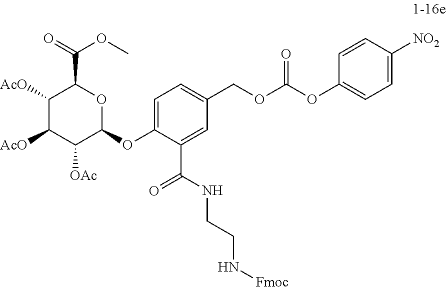

To the solution of 1-16d (140 mg, 0.19 mmol) in dry DMF (2 mL) were added bis(4-nitrophenyl) carbonate (69 mg, 0.22 mmol) and DIPEA (0.05 mL, 0.28 mmol) at 0° C. stirred at r.t. for 4 h. The resulting solution was quenched with sat. NH₄Cl and extracted with EtOAc (30 mL*3). The organic phase was concentrated and purified by silica gel column chromatography (EtOAc/Petroleum ether=80/20) to give 1-16e (110 mg, 64.4% yield) as a white solid. MS (ESI) m/z: 914.5 [M+H]

### Step 5: (2S,3R,4S,5S,6S)-2-(2-((2-((((9H-fluoren-9-yl)methoxy)carbonyl)amino)ethyl)carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-6-(methoxycarbonyl)tetrahydro-2H-pyran-3,4,5-triyl triacetate (1-16f)

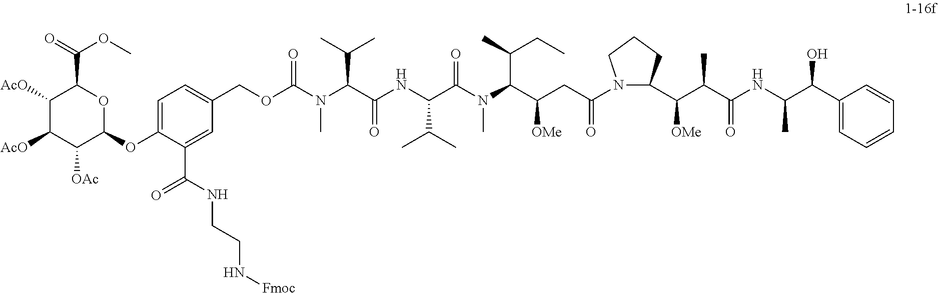

To the solution of 1-16e (110 mg, 0.12 mmol) and MMAE (96 mg, 0.13 mmol) in dry DMF (2 mL) were added HOBt (4.9 mg, 0.036 mmol) and DIPEA (0.65 mL, 0.36 mmol) at 0° C., stirred at r.t. for 24 h. The resulting solution was quenched with sat. NH₄Cl and extracted with EtOAc (30 mL*3). The organic phase was concentrated and purified by silica gel column chromatography (CH₂Cl/MeOH=95/5) to give 1-16f (110 mg, 61.2% yield) as a white solid. MS (ESI) m/z: 1494.3 [M+H]

### Step 6: (2S,3S,4S,5R,6S)-6-(2-((2-aminoethyl)carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (1-16g) trifluoroacetate

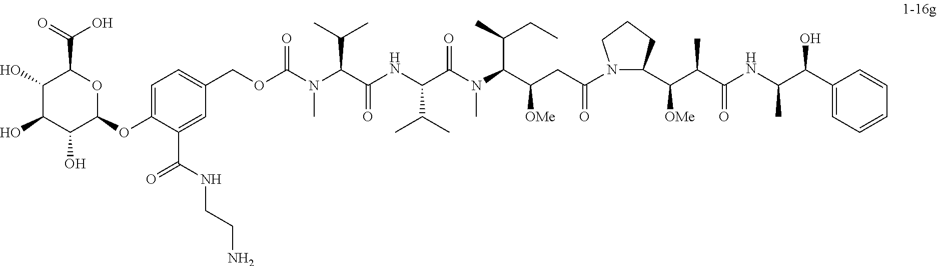 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2O[C@H](C(=O)O)[C@@H](O)[C@H](O)[C@H]2O)c(C(=O)NCCN)c1)C(C)C]

To the solution of 1-16f (110 mg, 0.074 mmol) in water (2 mL) and THF (2 mL) was added 1N LiOH (0.52 mL, 0.52 mmol) at 0° C., stirred at rt for 2 h. The pH was adjusted to around 4 by progressively adding FA. The resulting solution was purified by prep-HPLC (Method: column XBridge Prep C18 OBD 5 μm 19*150 mm; Mobile phase: A-water (0.1% TFA): B-acetonitrile; Flow rate: 20 mL/min, the fraction was lyophilized to give 1-16g (trifluoroacetate) (65 mg, 70.9% yield) as a white solid. MS (ESI) m/z: 1131.1 [M+H]⁺.

### Step 7: (2S,3S,4S,5R,6S)-6-(4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-(((R)-8-(2,5-dioxo-2,5-dihydro-1H-pyrrol-1-yl)-13,13-dimethyl-4,11-dioxo-5,12-dioxa-3,10-diazatetradecyl)carbamoyl)phenoxy)-3,4,5-trihydroxytetrahydro-2H-pyran-2-carboxylic acid (1-16h)

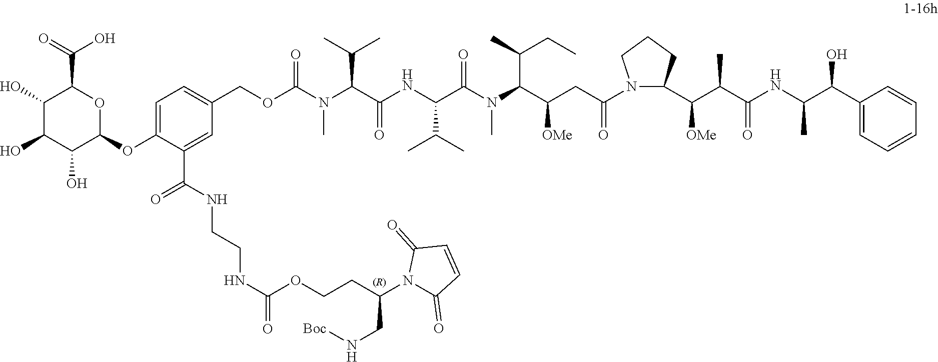

To the solution of 1-16g (trifluoroacetate) (35 mg, 0.028 mmol) and Int-2 (15 mg, 0.034 mmol) in dry DMF (2 mL) was added DIPEA (0.01 mL, 0.056 mmol) at 0° C., stirred at r.t. for 2 h. The resulting solution was purified by prep-HPLC (Method: column XBridge Prep C18 OBD 5 μm 19*150 mm; Mobile phase: A-water (0.1% FA): B-acetonitrile; Flow rate: 20 mL/min, the fraction was lyophilized to give 1-16h (23 mg, 56.8% yield) as a white solid. MS (ESI) m/z: 1441.4 [M+H]⁺.

### Step 8: (2S,3S,4S,5R,6S)-6-(2-((2-((((R)-4-amino-3-(2,5-dioxo-2,5-dihydro-1H-pyrrol-1-yl) butoxy)carbonyl)amino)ethyl)carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (1-16) trifluoroacetate

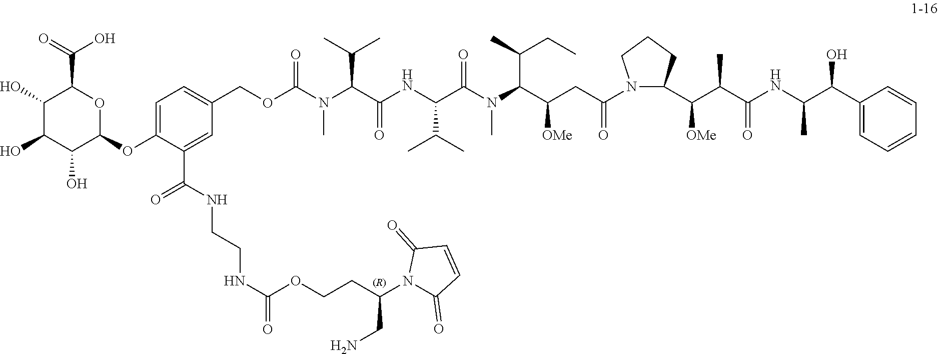

To the solution of 1-16h (23 mg, 0.016 mmol) in CH₂Cl₂ (1.2 mL) was added TFA (0.3 mL, 3.99 mmol) at 0° C., stirred at r.t. for 30 min. The resulting solution was purified by prep-HPLC (Method: column XBridge Prep C18 OBD 5 μm 19*150 mm; Mobile phase: A-water (0.1% TFA): B-acetonitrile; Flow rate: 20 mL/min, the fraction was lyophilized to give 1-16 (trifluoroacetate) (12.5 mg, 54.2% yield) as a white solid. MS (ESI) m/z: 1341.2 [M+H]⁺.

### Example 1-17

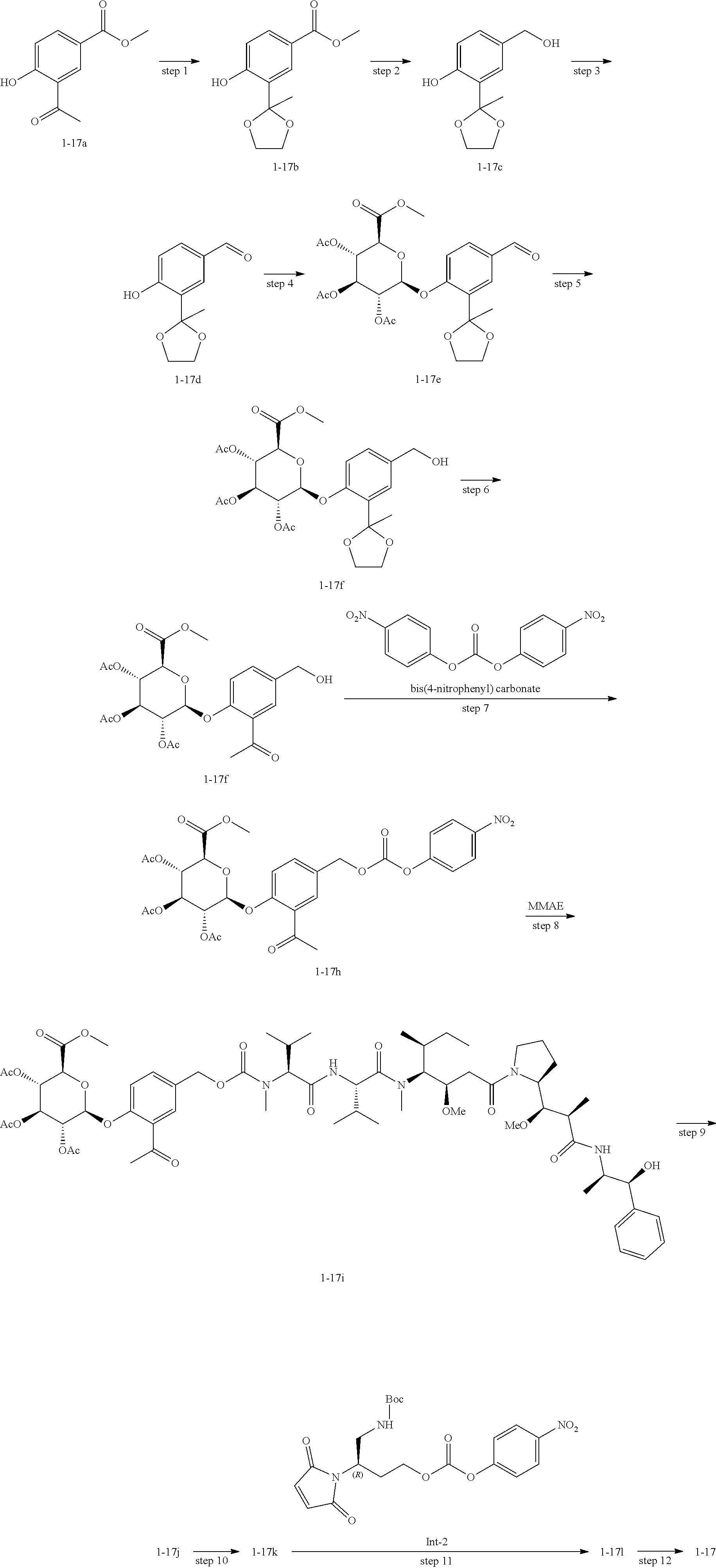

### Step 1: methyl 4-hydroxy-3-(2-methyl-1,3-dioxolan-2-yl)benzoate (1-17b)

To a solution of compound 1-17a (4.0 g, 20.6 mmol) in toluene (40 mL) was added PTSA (490 mg, 2.06 mmol), Ethylene glycol (8.5 mL, 206 mmol). The mixture was stirred at 110° C. for 10 h under N₂ atmosphere. The mixture was concentrated and purified by flash column chromatography (EtOAc/Petroleum ether=0-60%). Compound 1-17b (2.0 g, 40.7% yield) was obtained as a white solid. MS (ESI) m/z: 239.1 [M+H]⁺.

### Step 2:4-(hydroxymethyl)-2-(2-methyl-1,3-dioxolan-2-yl) phenol (1-17c)

To a solution of compound 1-17b (500 mg, 2.09 mmol) in THF (5.0 mL) was added LiAlH₄ (159 mg, 4.19 mmol) at 0° C. The mixture was stirred at 50° C. for 2 h. The mixture was quenched with water (160 μL), NaOH (160 μL, 15% in water) and H₂O (0.5 mL). The mixture was filtered and concentrated to give compound 1-17c (300 mg, 68.3% yield) as colorlees oil. MS (ESI) m/z: 211.2 [M+H]⁺.

### Step 3:4-hydroxy-3-(2-methyl-1,3-dioxolan-2-yl)benzaldehyde (1-17d)

To a solution of compound 1-17c (300 mg, 1.428 mmol) in EtOAc (5.0 mL) was added MnO₂ (1.24 g, 14.28 mmol) at 0° C. The mixture was stirred at room temperature for 5 h. The mixture was filtered, concentrated and purified by flash column chromatography (EtOAc/Petroleum ether=0-50%). Compound 1-17d (180 mg, 60.3% yield) was obtained as a white solid. MS (ESI) m/z: 209.1 [M+H]⁺.

### Step 4: (2S,3R,4S,5S,6S)-2-(4-formyl-2-(2-methyl-1,3-dioxolan-2-yl)phenoxy)-6-(methoxycarbonyl)tetrahydro-2H-pyran-3,4,5-triyl triacetate (1-17e)

To a solution of compound 1-17d (180 mg, 0.86 mmol) in MeCN (3.0 mL) was added (2R,3R,4S,5S,6S)-2-bromo-6-(methoxycarbonyl)tetrahydro-2H-pyran-3,4,5-triyl triacetate (515 mg, 1.29 mmol), 4Ams (300 mg) and Ag₂O (797 mg, 3.44 mmol). The mixture was stirred at room temperature for 10 h. The mixture was filtered, concentrated and purified by flash column chromatography (EtOAc/Petroleum ether=0-50%). Compound 1-17e (250 mg, 55.4% yield) was obtained as a white solid. MS (ESI) m/z: 525.7 [M+H]⁺.

### Step 5: (2S,3R,4S,5S,6S)-2-(4-(hydroxymethyl)-2-(2-methyl-1,3-dioxolan-2-yl)phenoxy)-6-(methoxycarbonyl)tetrahydro-2H-pyran-3,4,5-triyl triacetate (1-17f)

To a solution of compound 1-17e (250 mg, 0.477 mmol) in THF (5.0 mL) was added NaBH₄ (21 mg, 0.57 mmol) at 0° C. The mixture was stirred at 0° C. for 3 h. The mixture was quenched with sat. NH₄Cl, extracted with EtOAc (30 mL*3), concentrated and purified by flash column chromatography (EtOAc/Petroleum ether=0-70%). Compound 1-17f (180 mg, 60.3% yield) was obtained as a colorless oil. MS (ESI) m/z: 527.3 [M+H]⁺.

### Step 6: (2S,3R,4S,5S,6S)-2-(2-acetyl-4-(hydroxymethyl)phenoxy)-6-(methoxycarbonyl)tetrahydro-2H-pyran-3,4,5-triyl triacetate (1-17g)

To a solution of compound 1-17f (180 mg, 0.342 mmol) in DCM (3.0 mL) was added TFA (51 μL, 0.684 mmol) at 0° C. The mixture was stirred at r.t. for 3 h. The reaction was quenched with sat. NaHCO₃, extracted with EtOAc (30 mL*3), concentrated and purified by flash column chromatography (EtOAc/Petroleum ether=0-60%). Compound 1-17g (140 mg, 84.9% yield) was obtained as a colorless oil. MS (ESI) m/z: 483.5 [M+H]⁺.

### Step 7: (2S,3R,4S,5S,6S)-2-(2-acetyl-4-((((4-nitrophenoxy)carbonyl)oxy)methyl)phenoxy)-6-(methoxycarbonyl)tetrahydro-2H-pyran-3,4,5-triyl triacetate (1-17h)

To a solution of compound 1-17g (140 mg, 0.290 mmol) and bis(4-nitrophenyl) carbonate (132 mg, 0.435 mmol) in DCM (3.0 mL) was added DIPEA (101 μL, 0.58 mmol) at 0° C. The mixture was stirred at r.t. for 8 h. The reaction was concentrated and purified by flash column chromatography (EtOAc/Petroleum ether=0-50%). Compound 1-17h (140 mg, 74.5% yield) was obtained as a yellow solid. MS (ESI) m/z: 648.7 [M+H]⁺.

### Step 8: (2S,3R,4S,5S,6S)-2-(2-acetyl-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-6-(methoxycarbonyl)tetrahydro-2H-pyran-3,4,5-triyl triacetate (1-17i)

To a solution of compound 1-17h (140 mg, 0.216 mmol) and MMAE (155 mg, 0.216 mmol) in DMF (3.0 mL) was added DIPEA (113 μL, 0.648 mmol) and HOBt (7 mg, 0.054 mmol) at 0° C. The mixture was stirred at r.t. for 24 h. The reaction was concentrated and purified by flash column chromatography (MeOH/DCM=0-5%). Compound 1-17i (160 mg, 60.3% yield) was obtained as a White solid. MS (ESI) m/z: 1227.8 [M+H]⁺.

### Step 9: (2S,3R,4S,5S,6S)-2-(2-((E)-1-((2-aminoethoxy)imino)ethyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-6-(methoxycarbonyl)tetrahydro-2H-pyran-3,4,5-triyl triacetate (1-17j)

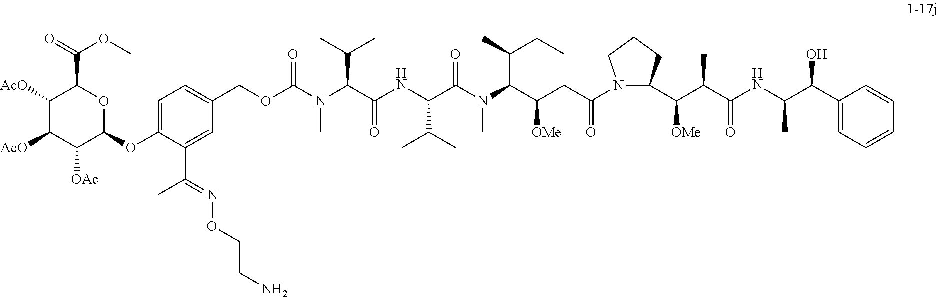 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2O[C@H](C(=O)OC)[C@@H](OC(C)=O)[C@H](OC(C)=O)[C@H]2OC(C)=O)c(/C(C)=N/OCCN)c1)C(C)C]

To a solution of compound 1-17i (160 mg, 0.130 mmol) in EtOH (3.0 mL) was added Ethanamine, 2-(aminooxy)-, dihydrochloride (97 mg, 0.65 mmol) at 0° C. The mixture was stirred at 50° C. for 5 h. The reaction was concentrated to give the crude product 1-17j. MS (ESI) m/z: 1284.8 [M+H]⁺.

### Step 10: (2S,3S,4S,5R,6S)-6-(2-((E)-1-((2-aminoethoxy)imino)ethyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (1-17k)

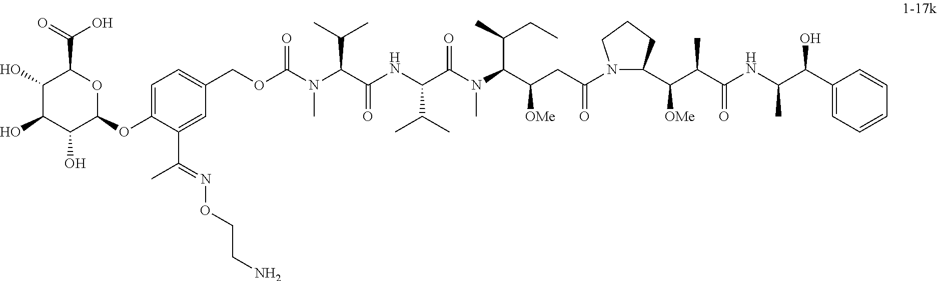 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2O[C@H](C(=O)O)[C@@H](O)[C@H](O)[C@H]2O)c(/C(C)=N/OCCN)c1)C(C)C]

To a solution of crude compound 1-17j in EtOH (3.0 mL) was added LiOH (780 μL, 0.78 mmol, 1 N in water) at 0° C. The mixture was stirred at r.t. for 0.5 h. The reaction was quenched with AcOH and purified by prep-HPLC (Method: column XBridge Prep C18 OBD 5 μm 19*150 mm; Mobile phase: A-water (0.1% TFA): B-acetonitrile; Flow rate: 20 mL/min, the fraction was lyophilized to give 1-17k (trifluoroacetate) (75 mg, 50.4% yield, two steps) as a white solid. MS (ESI) m/z: 1145.4 [M+H]⁺.

### Step 11: (2S,3S,4S,5R,6S)-6-(4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-((R,E)-12-(2,5-dioxo-2,5-dihydro-1H-pyrrol-1-yl)-17,17-dimethyl-8,15-dioxo-4,9,16-trioxa-3,7,14-triazaoctadec-2-en-2-yl)phenoxy)-3,4,5-trihydroxytetrahydro-2H-pyran-2-carboxylic acid 1-17l

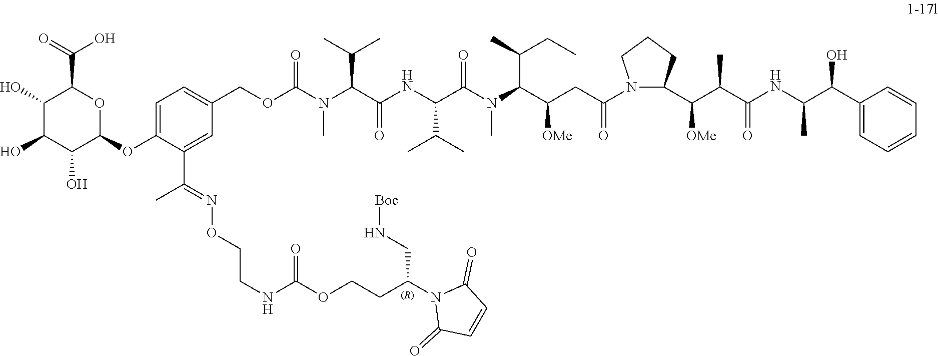

To a solution of crude compound 1-17k (30 mg, 0.0262 mmol) and Int-2 (13 mg, 0.0288 mmol) in DMF (1.0 mL) was added DIPEA (13 μL, 0.0786 mmol) at 0° C. The mixture was stirred at r.t. for 5 h. The reaction was purified by prep-HPLC (Method: column XBridge Prep C18 OBD 5 μm 19*150 mm; Mobile phase: A-water (0.1% TFA): B-acetonitrile; Flow rate: 20 mL/min, the fraction was lyophilized to give 1-17l (trifluoroacetate) (20 mg, 52.5% yield) as a white solid. MS (ESI) m/z: 1456.1 [M+H]⁺.

### Step 12: (2S,3S,4S,5R,6S)-6-(2-((R,E)-13-amino-12-(2,5-dioxo-2,5-dihydro-1H-pyrrol-1-yl)-8-oxo-4,9-dioxa-3,7-diazatridec-2-en-2-yl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid 1-17

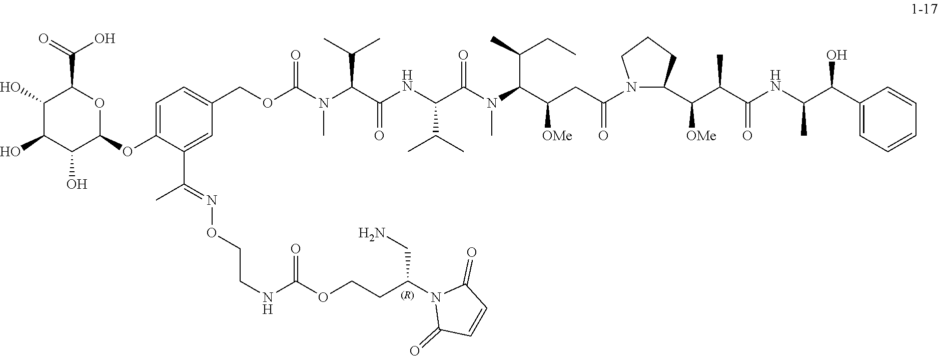

To a solution of crude compound 1-17l (20 mg, 0.0175 mmol) in DCM (1.0 mL) was added TFA (0.2 mL) at 0° C. The mixture was stirred at r.t. for 10 min. The reaction was purified by prep-HPLC (Method: column XBridge Prep C18 OBD 5 μm 19*150 mm; Mobile phase: A-water (0.1% TFA): B-acetonitrile; Flow rate: 20 mL/min. the fraction was lyophilized to give 1-17 (trifluoroacetate) (10 mg, 42.2% yield) as a white solid. MS (ESI) m/z: 1355.3 [M+H]⁺.

### Example 1-18

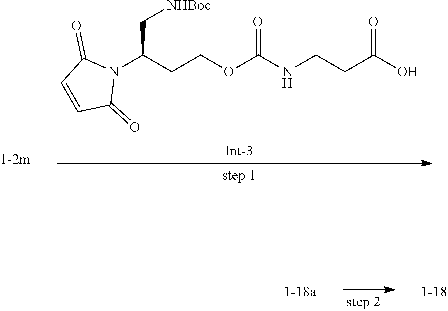 [SMILES: CC(C)(C)OC(=O)NC[C@@H](CCOC(=O)NCCC(=O)O)N1C(=O)C=CC1=O]

### Step 1: (7R,17S)-17-((3-((5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-(((2S,3R,4S,5S,6S)-6-carboxy-3,4,5-trihydroxytetrahydro-2H-pyran-2-yl)oxy)phenyl)amino)-3-oxopropyl)carbamoyl)-7-(2,5-dioxo-2,5-dihydro-1H-pyrrol-1-yl)-2,2,55,55-tetramethyl-4,11,15,23,51-pentaoxo-3,10,26,29,32,35,38,41,44,47-decaoxa-5,12,16,22,50,55-hexaazaoctapentacontan-55-ium-58-sulfonate (1-18a)

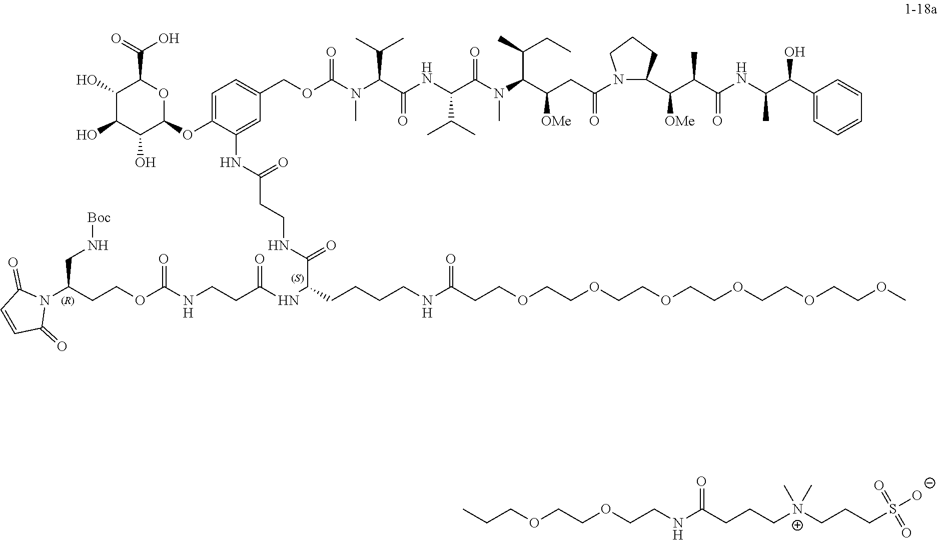

To the solution of Int-3 (25 mg, 0.063 mmol) in dry DMF (2 mL) were added HATU (24 mg, 0.063 mmol) and DIPEA (0.019 mL, 0.10 mmol), stirred at r.t. for 10 min. Then to the above mixture was added 1-2m (100 mg, 0.052 mmol), stirred at r.t. for 10 min. The resulting solution was purified by prep-HPLC (Method: column XBridge Prep C18 OBD 5 μm 19*150 mm; Mobile phase: A-water (0.1% FA): B-acetonitrile: Flow rate: 20 mL/min, the fraction was lyophilized to give 1-18a (55 mg, 45.9% yield) as a white solid. MS (ESI) m/z: 2298.9 [M+H]

### Step 2: (42S,52R)-53-amino-42-((3-((5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-(((2S,3R,4S,5S,6S)-6-carboxy-3,4,5-trihydroxytetrahydro-2H-pyran-2-yl)oxy)phenyl)amino)-3-oxopropyl)carbamoyl)-52-(2,5-dioxo-2,5-dihydro-1H-pyrrol-1-yl)-4,4-dimethyl-8,36,44,48-tetraoxo-12,15,18,21,24,27,30,33,49-nonaoxa-4,9,37,43,47-pentaazatripentacontan-4-ium-1-sulfonate (1-18)trifluoroacetate

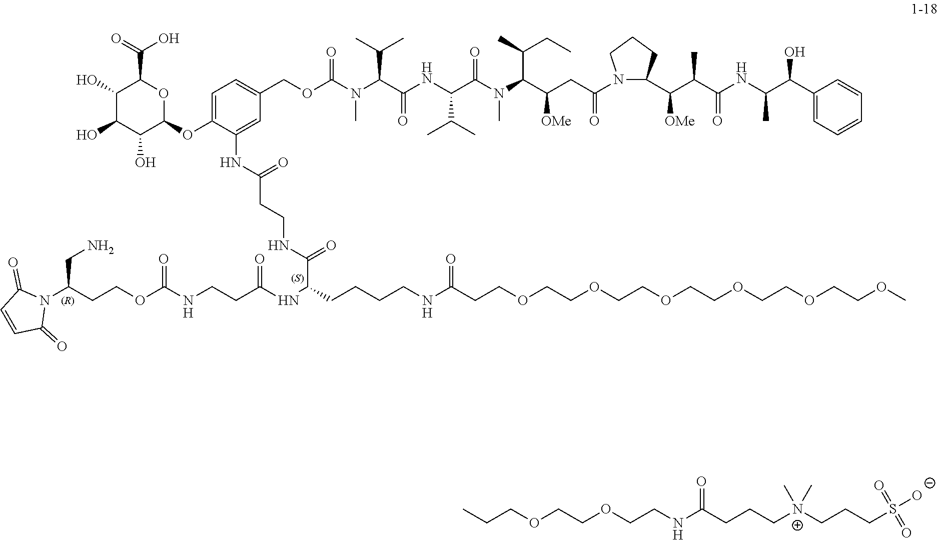

To the solution of 1-18a (55 mg, 0.024 mmol) in CH₂Cl₂ (1.2 mL) was added TFA (0.3 mL, 5.98 mmol) at 0° C., stirred at r.t. for 30 min. The resulting solution was purified by prep-HPLC (Method: column XBridge Prep C18 OBD 5 μm 19*150 mm; Mobile phase: A-water (0.1% TFA): B-acetonitrile; Flow rate: 20 mL/min, the fraction was lyophilized to give 1-18 (trifluoroacetate) (30 mg, 54.2% yield) as a white solid. MS (ESI) m/z: 2199.3 [M+H]⁺.

### Example 1-19

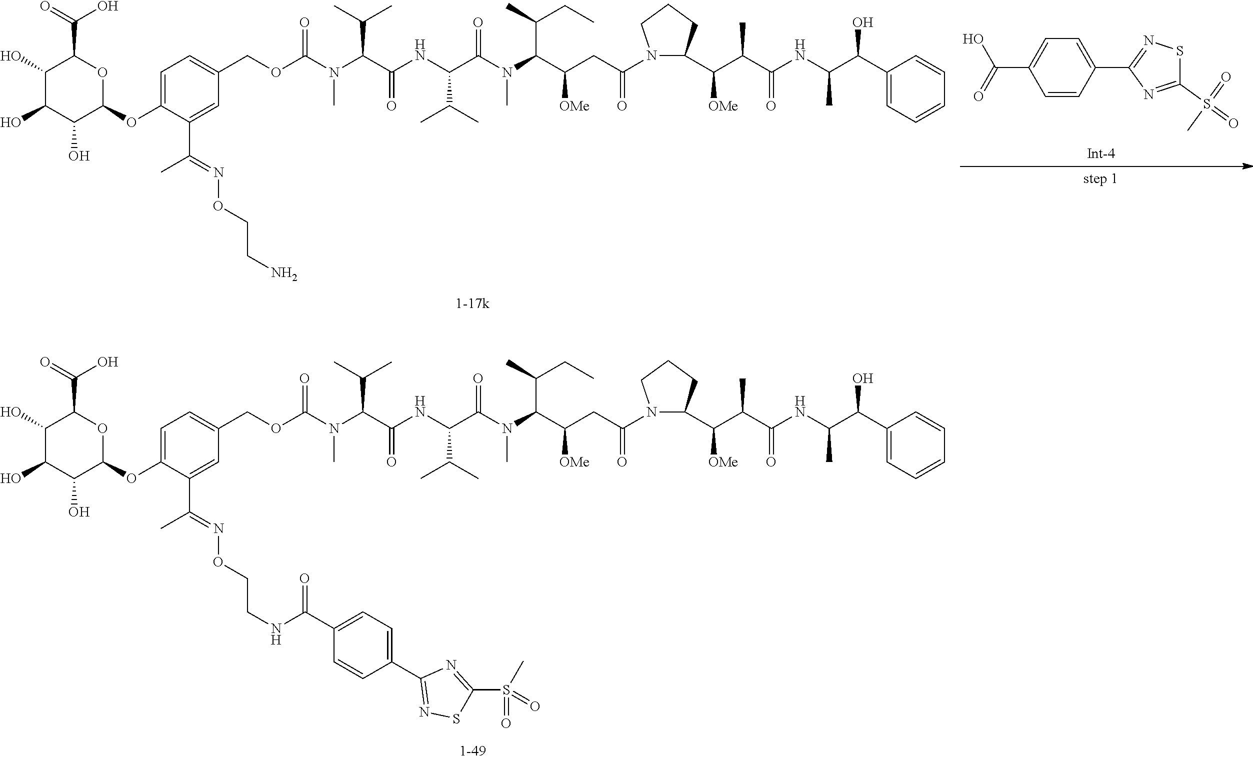

### Step 1: (2S,3S,4S,5R,6S)-6-(4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-((E)-1-((2-(4-(5-(methylsulfonyl)-1,2,4-thiadiazol-3-yl)benzamido) ethoxy)imino)ethyl)phenoxy)-3,4,5-trihydroxytetrahydro-2H-pyran-2-carboxylic acid (1-19)

To a solution of crude compound 1-17k (20 mg, 0.0175 mmol) and Int-4 (6 mg, 0.0192 mmol) in DMF (1.0 mL) was added HATU (7.3 mg, 0.0192 mmol) and DIPEA (9 μL, 0.0525 mmol) at 0° C. The mixture was stirred at r.t. for 1 h. The reaction was purified by prep-HPLC (Method: column XBridge Prep C18 OBD 5 μm 19*150 mm; Mobile phase: A-water (0.1% TFA): B-acetonitrile: Flow rate: 20 mL/min, the fraction was lyophilized to give 1-19 (trifluoroacetate) (10 mg, 40.5% yield) as a white solid. MS (ESI) m/z: 1411.4 [M+H]⁺.

### Example 1-20

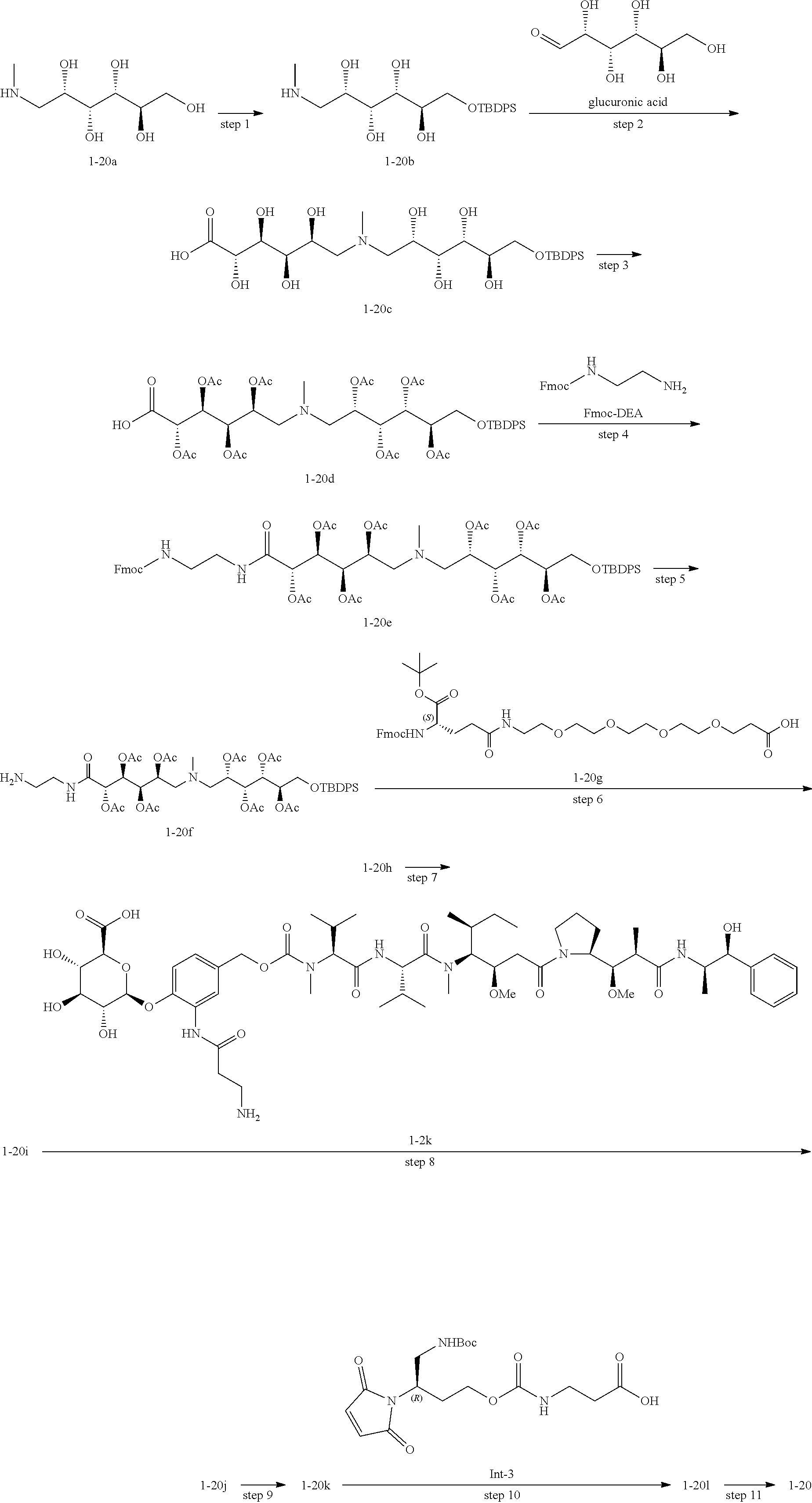

### Step 1: (2R,3R,4R,53)-1-(ter-butyldiphenylsilyl)oxy)-6-(methylamino) hexane-2.3.4.5-tetraol (1-20b)

To a solution of 1-20a (1.00 g, 5.1 mmol) and imidazole (0.37 g, 5.4 mmol) in DMF (10 mL) was added TBDPSCl at 0° C. The mixture was stirred at r.t. for 3 h and purified by flash column chromatography (MeOH/CH₂Cl₂=10/90) to give 1-20b as white solid (2.2 g, 99.0% yield). MS (ESI) m/z: 434.6 [M+H]⁺

### Step 2: (2S,3S,4R,5S)-6-(((2S,3R,4R,5R)-6-((tert-butyldiphenylsilyl)oxy)-2,3,4,5-tetrahydroxy hexyl)(methyl)amino)-2,3,4,5-tetrahydroxyhexanoic acid (1-20c)

To a solution of 1-20b (0.50 g, 1.2 mmol) and glucuronic acid (0.67 g, 3.5 mmol) in 10 mL MeOH was added NaCNBH₃. The mixture was stirred at r.t. at 60° C. for 4 h and quenched by aqueous NH₄Cl. The mixture was purified by prep-HPLC (Column: Xbridge Prep C18 OBD™ 5 μm, 19*150 mm; Mobile phase A: 0.1% FA in water, B: MeCN; Gradient: 20%-50% B; Flow rate: 20 mL/min), the fraction was lyophilized to give 1-20c (200 mg, 28.4% yield) as a white solid. MS (ESI) m/z: 612.7 [M+H]⁺

### Step 3: (2S,3S,4R,5S)-2,3,4,5-tetraacetoxy-6-(methyl((2S,3R,4R,5R)-2,3,4,5-tetraacetoxy-6-((tert-butyldiphenylsilyl)oxy) hexyl)amino) hexanoic acid (1-20d)

To a solution of 1-20c (200 mg, 0.33 mmol) in acetic anhydride (5 mL) was added DMAP (40 mg, 0.33 mmol). The mixture was stirred at r.t. for 2 h and purified by flash column chromatography (Petroleum ether/EtOAc=50/50) to give 1-20d as a white solid (200 mg, 64.5% yield). MS (ESI) m/z: 948.9 [M+H]⁺

### Step 4: (9S,10S,11R,12S,16S,17R,18R,19R)-1-(9H-fluoren-9-yl)-14,23,23-trimethyl-3,8-dioxo-22,22-diphenyl-2,21-dioxa-4,7,14-triaza-22-silatetracosan-9,10,11,12,16,17,18,19-octayl octaacetate (1-20e)

To a solution of 1-20d (200 mg, 0.21 mmol) and Fmoc-DEA (80 mg, 0.25 mmol) in 2 mL dry DMF was added DIPEA (136 μL, 0.78 mmol) and HATU (160 mg, 0.42 mmol). The mixture was stirred at r.t. for 3 h. The mixture was partitioned between water and ethyl acetate. The organic layer was concentrated and purified by flash column chromatography (Petroleum ether/EtOAc=34/66) to give 1-20e as a white solid (250 mg, 97.8% yield). MS (ESI) m/z: 1213.6 [M+H]⁺

### Step 5: (6R,7R,8R,9S,13S,14R,15S,16S)-20-amino-2,2,11-trimethyl-17-oxo-3,3-diphenyl-4-oxa-11,18-diaza-3-silaicosan-6,7,8,9,13,14,15,16-octayl octaacetate (1-20f)

To a solution of 1-20e (275 mg, 0.23 mmol) in 2 mL dry THF was added 200 μL piperidine. The mixture was stirred at r.t. for 3 h and was concentrated to give crude product 1-20f without further purification. MS (ESI) m/z: 991.1 [M+H]⁺

### Step 6: (5S,30S,31S,32R,33S,37S,38R,39R,40R)-5-(tert-butoxycarbonyl)-1-(9H-fluoren-9-yl)-35,44,44-trimethyl-3,8,24,29-tetraoxo-43,43-diphenyl-2,12,15,18,21,42-hexaoxa-4,9,25,28,35-pentaaza-43-silapentatetracontan-30,31,32,33,37,38,39,40-octayl octaacetate (1-20h)

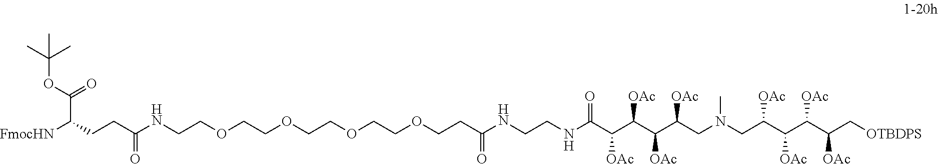

To a solution of 1-20f, 1-20g (150 mg, 0.22 mmol) and DIPEA (78 μL, 0.45 mmol) in dry DMF (2 mL) was added HATU (127 mg, 0.33 mmol). The mixture was stirred at r.t. for 3 h and was purified by flash column chromatography (MeOH/CH₂Cl₂=10/90) to give 1-20h as a white solid (189 mg, 51.5% yield in two steps). MS (ESI) m/z: 1646.3 [M+H]⁺

### Step 7: (6R,7R,8R,9S,13S,14R,15S,16S,41S)-41-((((9H-fluoren-9-yl)methoxy)carbonyl)amino)-6,7,8,9,13,14,15,16-octaacetoxy-2,2,11-trimethyl-17,22,38-trioxo-3,3-diphenyl-4,25,28,31,34-pentaoxa-11,18,21,37-tetraaza-3-siladotetracontan-42-oic acid (1-20i)

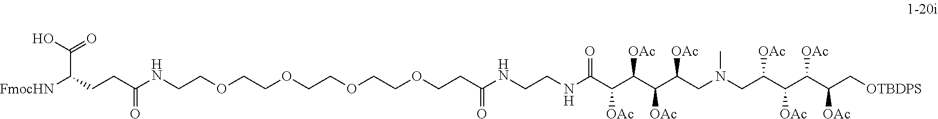

To a solution of 1-20h (174 mg, 0.11 mmol) in 2 mL dry CH₂Cl₂ was added 400 μL. TFA. The mixture was stirred at r.t. for 3 h and was concentrated to give crude product 1-20i without further purification. MS (ESI) m/z: 1587.8 [M−H⁺]

### Step 8: (2S,3S,4S,5R,6S)-6-(2-((6R,7R,8R,9S,13S,14R,15S,16S,41S)-41-((((9H-fluoren-9-yl)methoxy)carbonyl)amino)-6,7,8,9,13,14,15,16-octaacetoxy-2,2,11-trimethyl-17,22,38,42-tetraoxo-3,3-diphenyl-4,25,28,31,34-pentaoxa-11,18,21,37,43-pentaaza-3-silahexatetracontan-46-amido)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (1-20j)

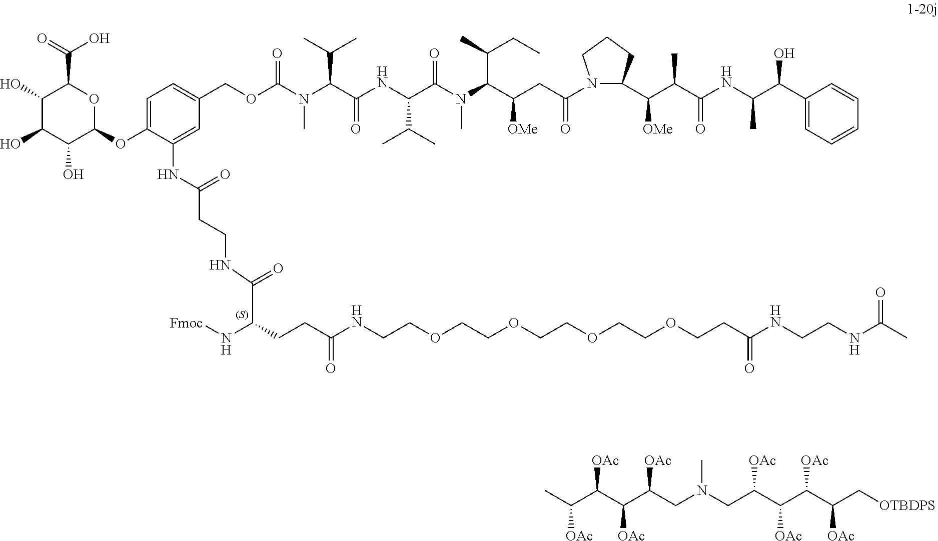

To a solution of 1-20i, HATU (40 mg, 0.11 mmol) and DIPEA (37 μL, 0.21 mmol) in dry DMF (2 mL) was added 1-2k (120 mg, 0.11 mmol). The mixture was stirred at r.t. for 6 h and was purified by prep-HPLC (Column: Xbridge Prep C18 OBD™ 5 μm, 19*150 mm; Mobile phase A: 0.1% FA in water. B: MeCN; Gradient: 25%-60% B: Flow rate: 20 mL/min), the fraction was lyophilized to give 1-20j as a white solid (44 mg, 15.4% yield in two steps). MS (ESI) m/z: 2699.3 [M+H]⁺

### Step 9: (2S,3S,4S,5R,6S)-6-(2-((6S,31S,32S,33R,34S,38S,39R,40R,41R)-6-amino-31,32,33,34,38,39,40,41,42-nonahydroxy-36-methyl-5,9,25,30-tetraoxo-13, 16, 19,22-tetraoxa-4,10,26,29,36-pentaazadotetracontanamido)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (1-20k)

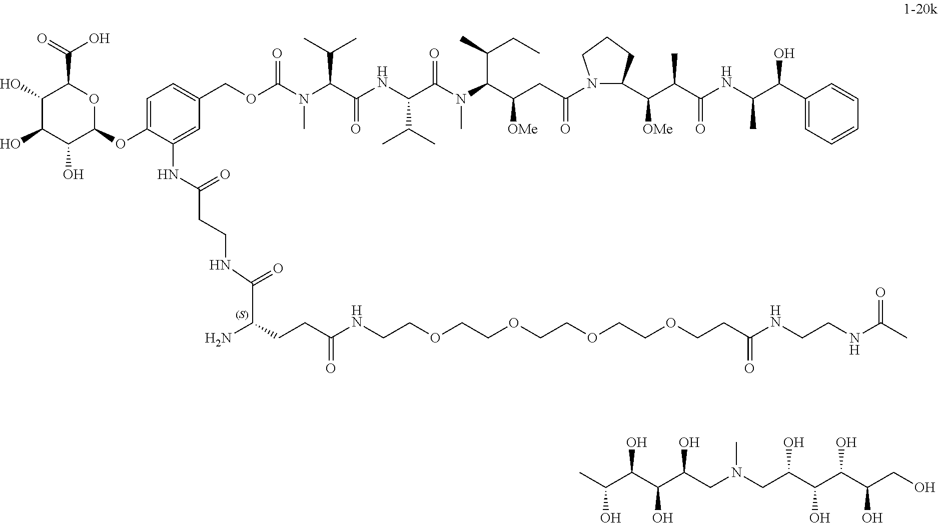

To a solution of 1-20j (44 mg, 0.016 mmol) in 2 mL dry THF was added TBAF. The mixture was stirred at 40° C. for 3 h. Then 1N LiOH was added, the mixture was stirred at r.t. further for 20 min and quenched with 20 μL AcOH. The mixture was purified by prep-HPLC (Column: Xbridge Prep C18 OBD™ 5 μm, 19*150 mm; Mobile phase A: 0.1% FA in water. B: MeCN; Gradient: 20%-55% B; Flow rate: 20 mL/min), the fraction was lyophilized to give 1-20k as a white solid (20 mg, 64.5% yield). MS (ESI) m/z: 1905.5 [M+H]⁺

### Step 10: (2S,3S,4S,5R,6S)-6-(4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-((6S,31S,32S,33R,34S,38S,39R,40R,41R)-6-((R)-7-(2,5-dioxo-2,5-dihydro-1H-pyrrol-1-yl)-2,2-dimethyl-4,11-dioxo-3,10-dioxa-5,12-diazapentadecan-15-amido)-31,32,33,34,38,39,40,41,42-nonahydroxy-36-methyl-5,9,25,30-tetraoxo-13,16,19,22-tetraoxa-4,10,26,29,36-pentaazadotetracontanamido)phenoxy)-3,4,5-trihydroxytetrahydro-2H-pyran-2-carboxylic acid (1-20l)

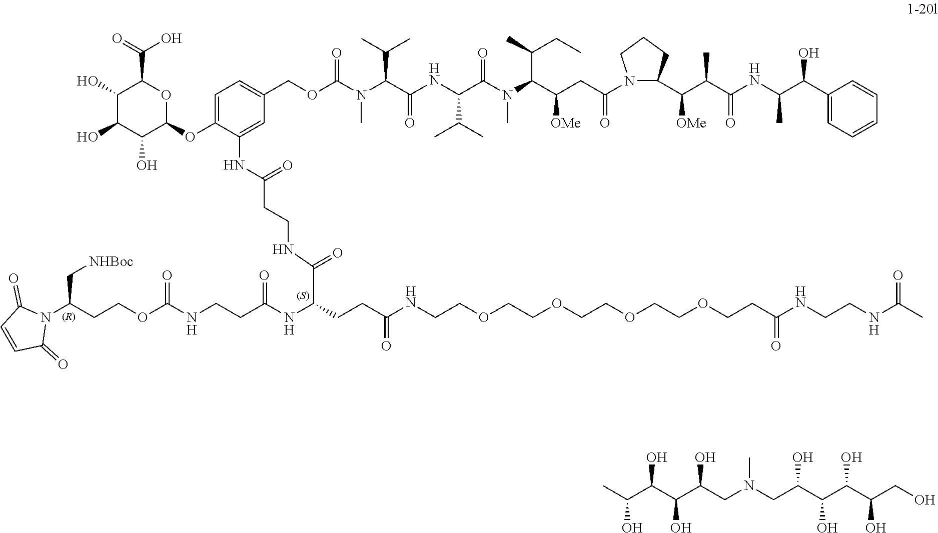

To a solution of Int-3 (4.3 mg, 0.011 mmol), HATU (4.1 mg, 0.011 mmol) and DIPEA (2.8 μL, 0.016 mmol) in dry DMF (2 mL) was added 1-20k (20 mg, 0.011 mmol). The mixture was stirred at r.t. for 1 h and purified by prep-HPLC (Column: Xbridge Prep C18 OBD™ 5 μm, 19*150 mm; Mobile phase A: 0.1% FA in water, B: MeCN; Gradient: 25%-65% B; Flow rate: 20 ml/min), the fraction was lyophilized to give 1-20l as a white solid (8.4 mg, 34.1% yield). MS (ESI) m/z: 2286.5 [M+H]⁺

### Step 11: (2S,3S,4S,5R,6S)-6-(2-((6S,31S,32S,33R,34S,38S,39R,40R,41R)-6-(3-((((R)-4-amino-3-(2,5-dioxo-2,5-dihydro-1H-pyrrol-1-yl) butoxy)carbonyl)amino)propanamido)-31,32,33,34,38,39,40,41,42-nonahydroxy-36-methyl-5,9,25,30-tetraoxo-13, 16, 19,22-tetraoxa-4,10,26,29,36-pentaazadotetracontanamido)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (1-20)

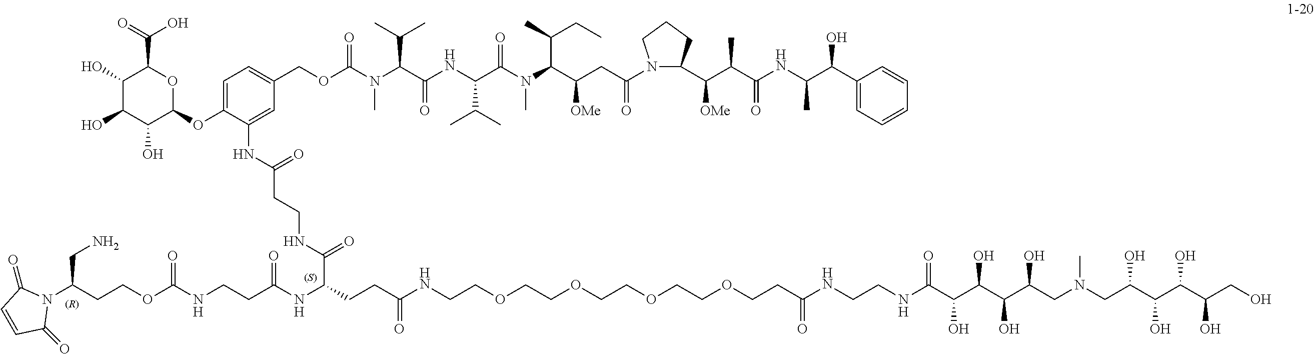

To a solution of 1-20l (8.4 mg, 3.7 μmol) in dry CH₂Cl₂ (1 mL) was added TFA (200 μL). The mixture was stirred at r.t. for 30 min and purified by prep-HPLC (Column: Xbridge Prep C18 OBD™ 5 μm, 19*150 mm; Mobile phase A: 0.1% TFA in water. B: MeCN; Gradient: 25%-55% B: Flow rate: 20 mL/min), the fraction was lyophilized to give 1-20 as a white solid (5.5 mg, 68.5% yield). MS (ESI) m/z: 2186.9 [M+H]⁺

### Example 1-21

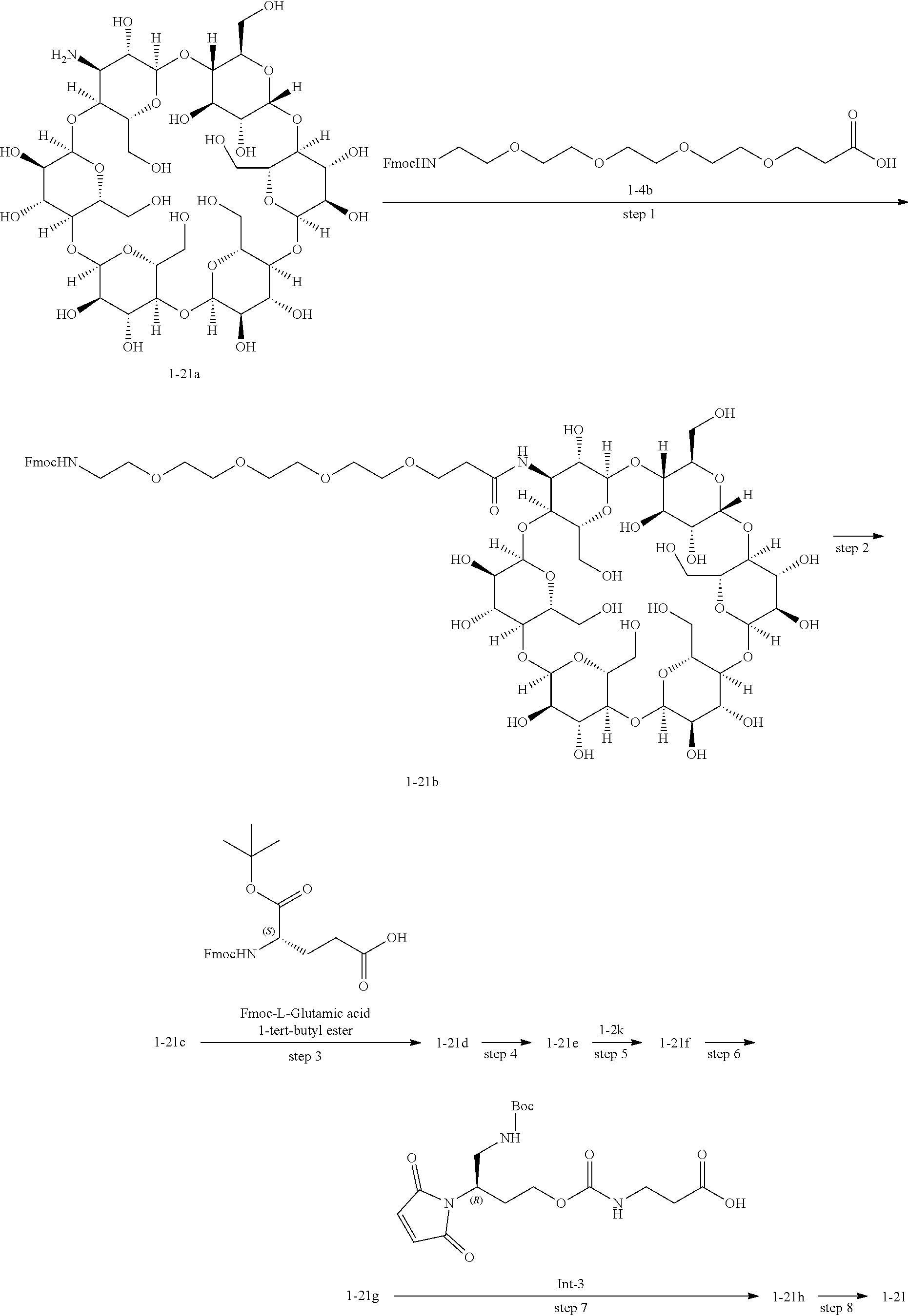

### Step 1: Synthesis of Compound 1-21b

To the solution of 1-21a (80 mg, 0.082 mmol) and 1-4b (45 mg, 0.091 mmol) in dry DMF (1 mL) were added HATU (38 mg, 0.099 mmol) and DIPEA (0.045 mL, 0.25 mmol), stirred at r.t. for 10 min. The resulting solution was purified by prep-HPLC (Method: column XBridge Prep C18 OBD 5 μm 19*150 mm; Mobile phase: A-water (0.1% FA): B-acetonitrile; Flow rate: 20 mL/min, the fraction was lyophilized to give 1-21b (89 mg, 75% yield) as a white solid. MS (ESI) m/z: 1442.5 [M+H]⁺

### Step 2: Synthesis of Compound 1-21c

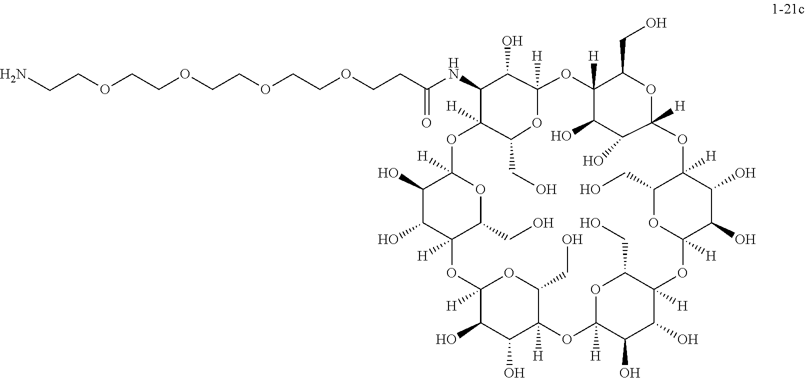 [SMILES: NCCOCCOCCOCCOCCC(=O)N[C@H]1[C@H](O)[C@H]2O[C@H]3[C@H](O)[C@@H](O)[C@@H](O[C@H]4[C@H](O)[C@@H](O)[C@@H](O[C@H]5[C@H](O)[C@@H](O)[C@@H](O[C@H]6[C@H](O)[C@@H](O)[C@@H](O[C@H]7[C@H](O)[C@@H](O)[C@@H](O[C@@H]1[C@@H](CO)O2)O[C@@H]7CO)O[C@@H]6CO)O[C@@H]5CO)O[C@@H]4CO)O[C@@H]3CO]

To the solution of 1-21b (69 mg, 0.026 mmol) in dry DMF (2 mL) was added Et₂NH (0.19 mL, 1.85 mmol), stirred at r.t. for 30 min. The resulting solution was concentrated to give 1-21c (75 mg, 99.6% yield) crude product, which was directly used in the next step. MS (ESI) m/z: 1220.2 [M+H]⁺

### Step 3: Synthesis of Compound 1-21d

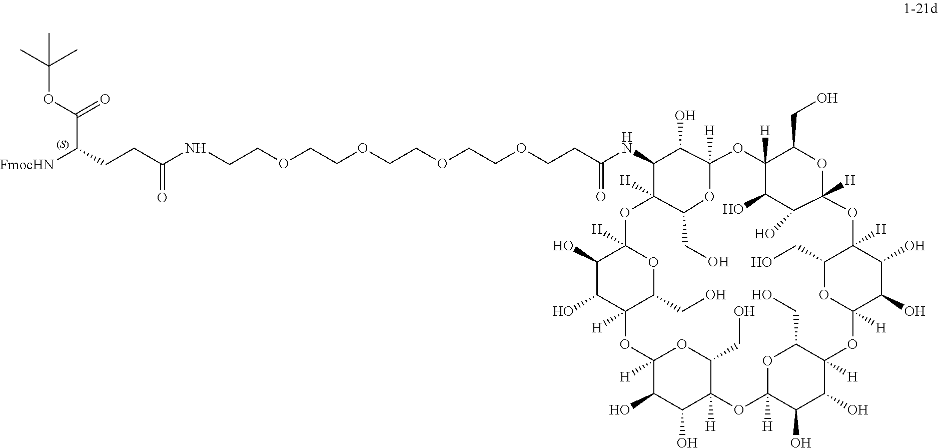

To the solution of 1-21c (75 mg, 0.062 mmol) and Fmoc-L-Glutamic acid 1-tert-butyl ester (29 mg, 0.068 mmol) in dry DMF (1 mL) were added HATU (28 mg, 0.074 mmol) and DIPEA (0.033 mL, 0.18 mmol), stirred at r.t. for 10 min. The resulting solution was purified by prep-HPLC (Method: column XBridge Prep C18 OBD 5 μm 19*150 mm; Mobile phase: A-water (0.1% FA): B-acetonitrile; Flow rate: 20 mL/min, the fraction was lyophilized to give 1-21d (79 mg, 79% yield) as a white solid. MS (ESI) m/z: 1627.7 [M+H]⁺

### Step 4: Synthesis of Compound 1-21e

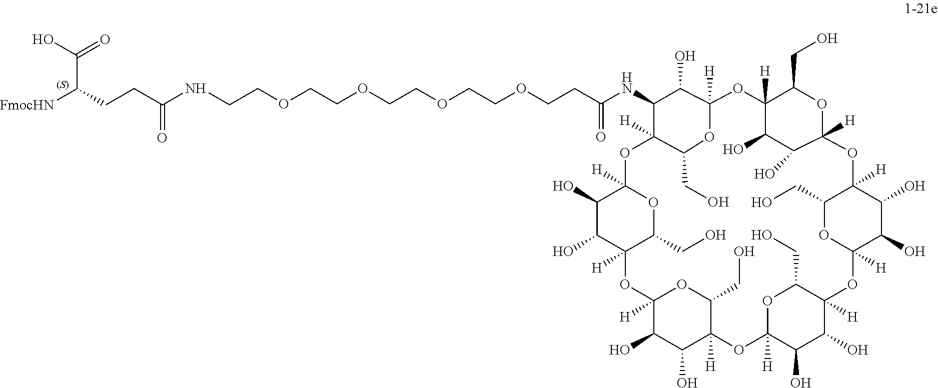

To the solution of 1-21d (79 mg, 0.049 mmol) in DCM (2 mL) was added TFA (0.36 mL, 4.86 mmol), stirred at r.t. for 3 h. The mixture was concentrated to remove solvent. The residue was dissolved with THF (1 mL) and MeOH (1 mL). To the above mixture was added 1N LiOH (0.73 mL, 0.33 mmol) and 30% H₂O₂ (0.31 mL, 1.33 mmol), stirred at 0° C. for 1 h. The resulting solution was quenched with AcOH and sat. Na₂S₂O₃, concentrated to remove solvent. The residue was purified by prep-HPLC (Method: column XBridge Prep C18 OBD 5 um 19*150 mm; Mobile phase: A-water (0.1% FA): B-acetonitrile; Flow rate: 20 mL/min, the fraction was lyophilized to give 1-21e (58 mg, 76% yield) as a white solid. MS (ESI) m/z: 1571.9 [M+H]

### Step 5: Synthesis of Compound 1-21f

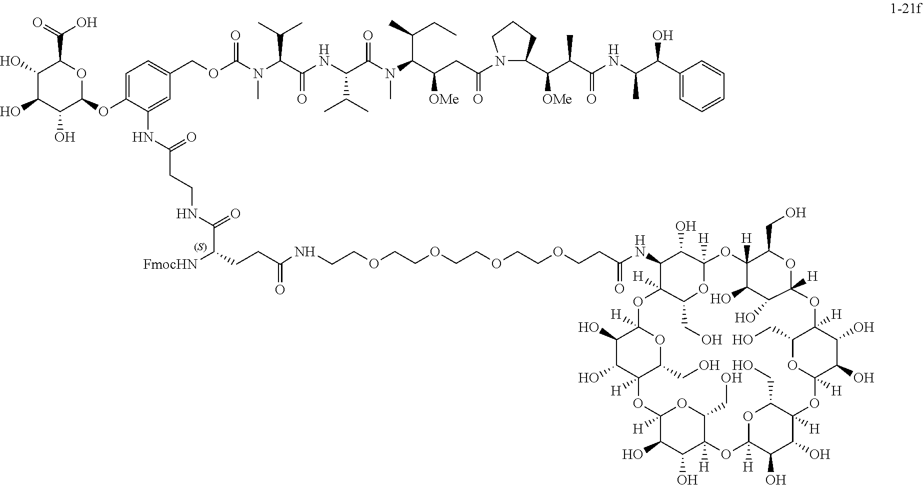

To the solution of 1-21e (58 mg, 0.037 mmol) in dry DMF (1 mL) were added HATU (14 mg, 0.037 mmol) and DIPEA (0.02 mL, 0.11 mmol), stirred at r.t. for 10 min. To the above mixture was added 1-2k (46 mg, 0.037 mmol), stirred at r.t. for 10 min. The resulting solution was purified by prep-HPLC (Method: column XBridge Prep C18 OBD 5 μm 19*150 mm; Mobile phase: A-water (0.1% FA): B-acetonitrile; Flow rate: 20 mL/min, the fraction was lyophilized to give 1-21f (69 mg, 69.6% yield) as a white solid. MS (ESI) m/z: 2682.5 [M+H]⁺

### Step 6: Synthesis of Compound 1-21g

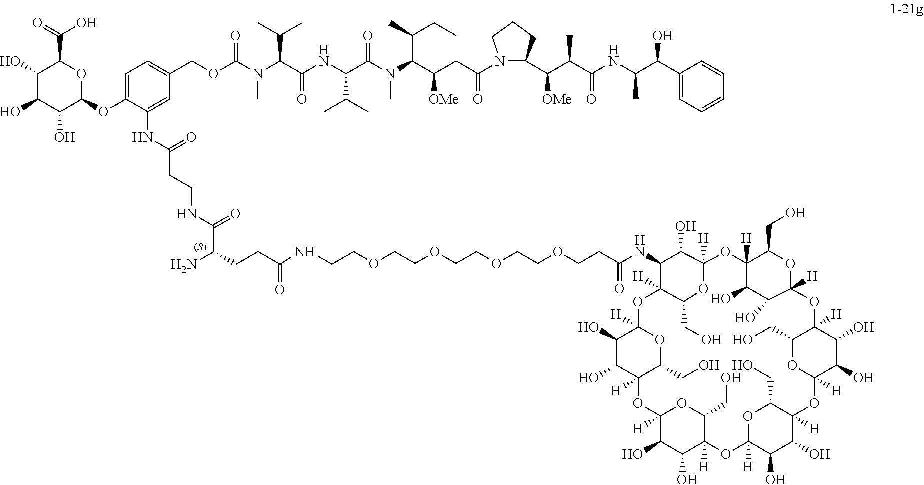

To the solution of 1-21f (69 mg, 0.026 mmol) in dry DMF (2 mL) was added Et₂NH (0.081 mL, 0.77 mmol), stirred at r.t. for 30 min. The resulting solution was concentrated to give 1-21g (60 mg, 94.8% yield) crude product, which was directly used in the next step. MS (ESI) m/z: 2461.0 [M+H]⁺

### Step 7: Synthesis of Compound 1-21h

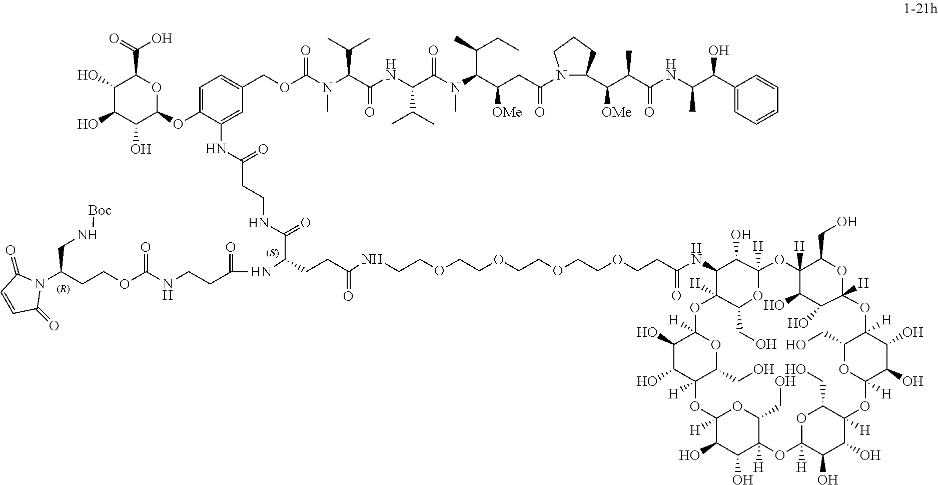

To the solution of Int-3 (10 mg, 0.024 mmol) in dry DMF (1 mL) were added HATU (10 mg, 0.024 mmol) and DIPEA (0.013 mL, 0.073 mmol), stirred at r.t. for 10 min. To the above mixture was added 1-21g (60 mg, 0.024 mmol), stirred at r.t. for 10 min. The resulting solution was purified by prep-HPLC (Method: column XBridge Prep C18 OBD 5 μm 19*150 mm; Mobile phase: A-water (0.1% FA): B-acetonitrile; Flow rate: 20 mL/min, the fraction was lyophilized to give 1-21h (40 mg, 57.7% yield) as a white solid. MS (ESI) m/z: 1422.3 [M/2+H]⁺

### Step 8: Synthesis of Compound 1-21

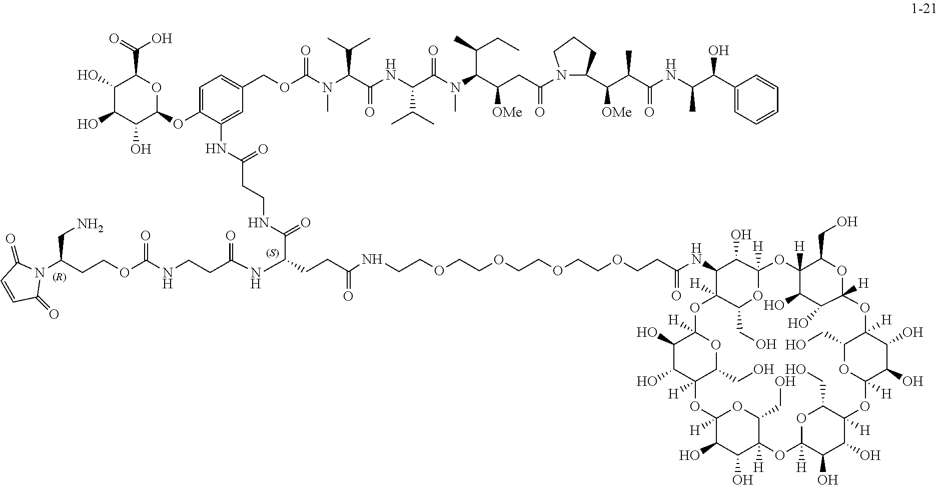

To the solution of 1-21h (40 mg, 0.014 mmol) in DCM (2 mL) was added TFA (0.38 mL, 4.93 mmol), stirred at r.t. for 30 min. The resulting solution was concentrated to remove solvent. The residue was purified by prep-HPLC (Method: column XBridge Prep C18 OBD 5 μm 19*150 mm; Mobile phase: A-water (0.05% TFA): B-acetonitrile; Flow rate: 20 mL/min, the fraction was lyophilized to give 1-21 (trifluoroacetate) (21 mg, 52.2% yield) as a white solid. MS (ESI) m/z: 1372.4 [M/2+H]⁺

### Example 1-22

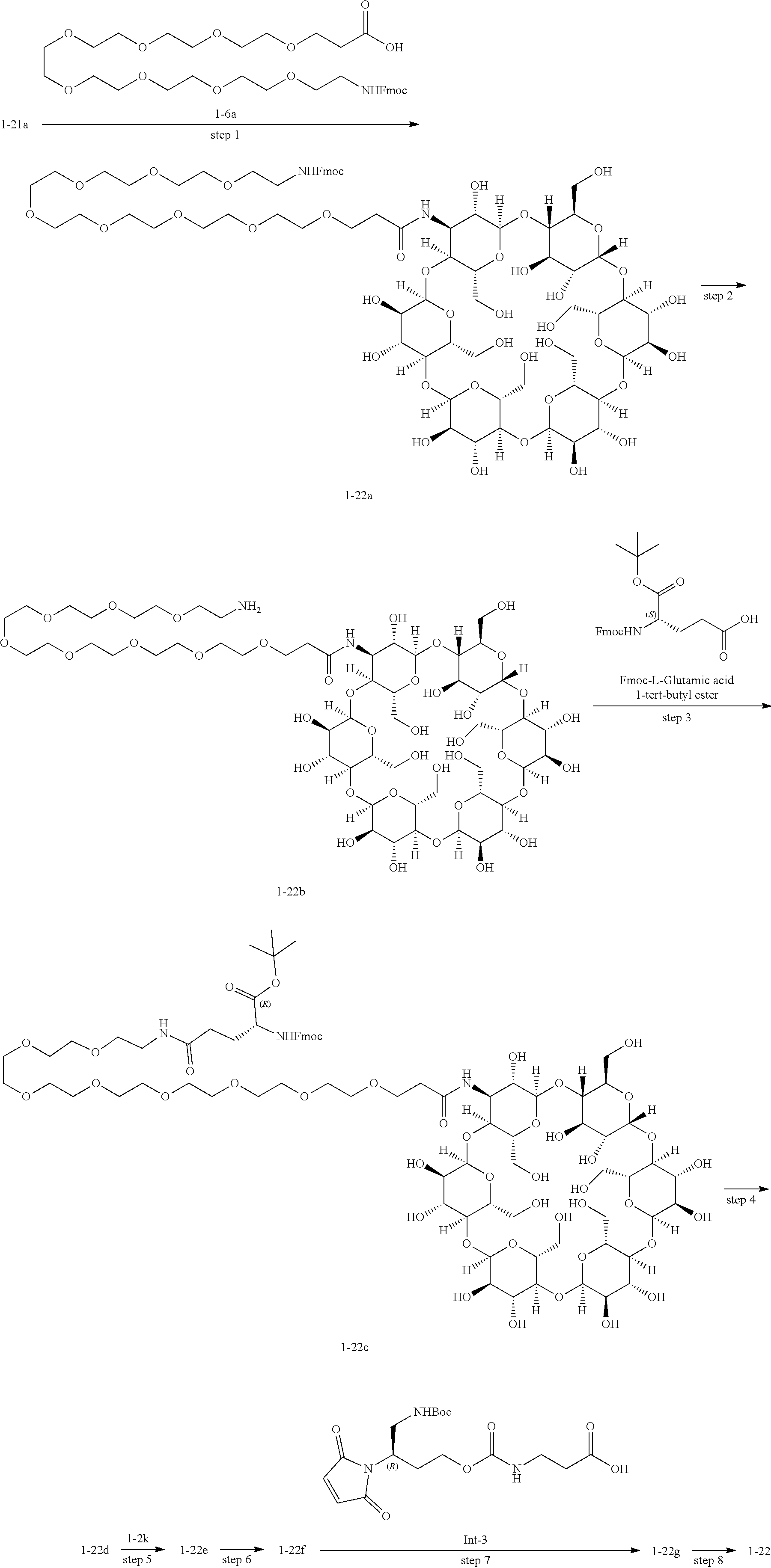

### Step 1: Synthesis of Compound 1-22a

To the solution of 1-6a (48 mg, 0.072 mmol) and 1-21a (70 mg, 0.072 mmol) in dry DMF (2 mL) were added HATU (39 mg, 0.1 mmol) and DIPEA (0.033 mL, 0.18 mmol), stirred at r.t. for 1 h. The resulting solution was purified by prep-HPLC (Method: column XBridge Prep C18 OBD 5 μm 19*150 mm; Mobile phase: A-water (0.1% FA): B-acetonitrile: Flow rate: 20 mL/min, the fraction was lyophilized to give 1-22a (90 mg, 77.2% yield) as a colorless oil. MS (ESI) m/z: 1618.6 [M+H]⁺

### Step 2: Synthesis of Compound 1-22b

To the solution of 1-22a (90 mg, 0.056 mmol) in dry DMF (2 mL) was added Et₂NH (0.18 mL, 1.67 mmol), stirred at r.t. for 30 min. The resulting solution was concentrated to give 1-22b (78 mg, 100.5% yield) crude product, which was directly used in the next step. MS (ESI) m/z: 1396.0 [M+H]⁺

### Step 3: Synthesis of Compound 1-22c

To the solution of 1-22b (78 mg, 0.056 mmol) and Fmoc-L-Glutamic acid 1-tert-butyl ester (26 mg, 0.061 mmol) in dry DMF (2 mL) were added HATU (26 mg, 0.067 mmol) and DIPEA (0.025 mL, 0.14 mmol), stirred at r.t. for 1 h. The resulting solution was purified by prep-HPLC (Method: column XBridge Prep C18 OBD 5 μm 19*150 mm; Mobile phase: A-water (0.1% FA): B-acetonitrile; Flow rate: 20 mL/min. the fraction was lyophilized to give 1-22c (63 mg, 62.5% yield) as a colorless oil. MS (ESI) m/z: 1804.0 [M+H]⁺

### Step 4: Synthesis of Compound 1-22d

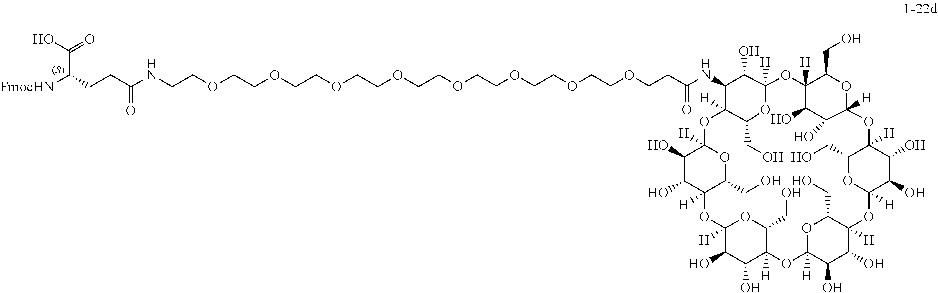

To the solution of 1-22c (40 mg, 0.022 mmol) in DCM (2 mL) was added TFA (0.17 mL, 2.22 mmol), stirred at r.t. for 3 h. The mixture was concentrated to remove solvent. The residue was dissolved with THF (1 mL) and MeOH (1 mL). To the above mixture was added 1N LiOH (0.29 mL, 0.33 mmol) and H₂O₂ (0.14 mL, 1.33 mmol) at 0° C., stirred at 0° C. for 1 h. The resulting solution was quenched with sat. Na₂S₂O₃ and AcOH, concentrated to remove solvent. The residue was purified by prep-HPLC (Method: column XBridge Prep C18 OBD 5 μm 19*150 mm; Mobile phase: A-water (0.1% FA): B-acetonitrile; Flow rate: 20 mL/min, the fraction was lyophilized to give 1-22d (25 mg, 64.5% yield) as a colorless oil. MS (ESI) m/z: 1747.7 [M+H]

### Step 5: Synthesis of Compound 1-22e

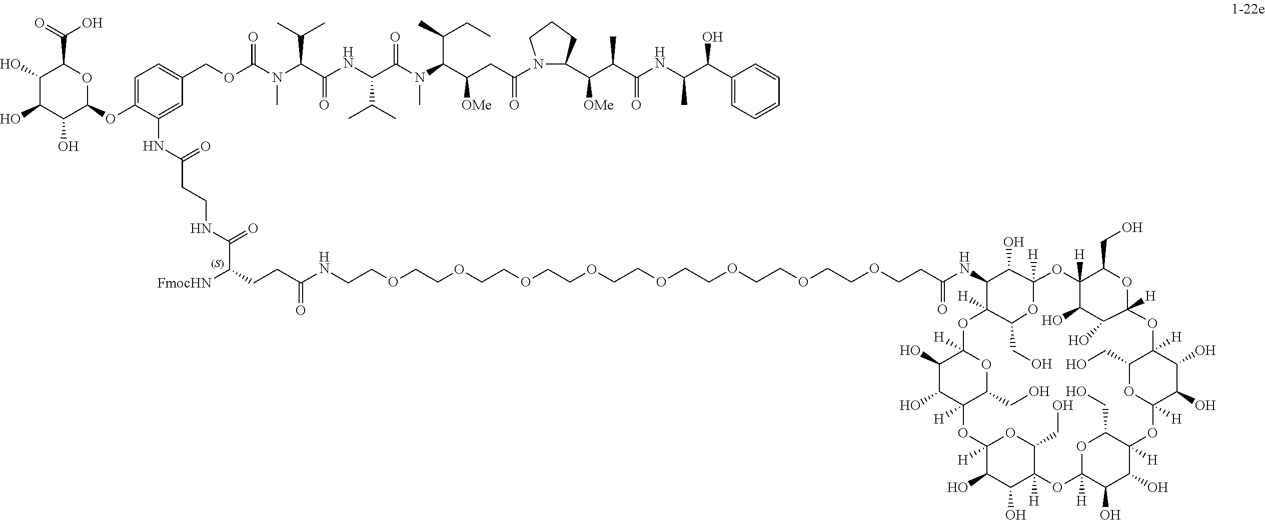

To the solution of 1-22d (25 mg, 0.014 mmol) in dry DMF (1 mL) were added HATU (6 mg, 0.016 mmol) and DIPEA (0.005 mL, 0.029 mmol), stirred at r.t. for 10 min. To the above mixture was added 1-2k (20 mg, 0.016 mmol), stirred at r.t. for 10 min. The resulting solution was purified by prep-HPLC (Method: column XBridge Prep C18 OBD 5 μm 19*150 mm; Mobile phase: A-water (0.1% FA): B-acetonitrile; Flow rate: 20 mL/min, the fraction was lyophilized to give 1-22e (12 mg, 62.5% yield) as a white solid. MS (ESI) m/z: 1430.4 [M/2+H]⁺

### Step 6: Synthesis of Compound 1-22f

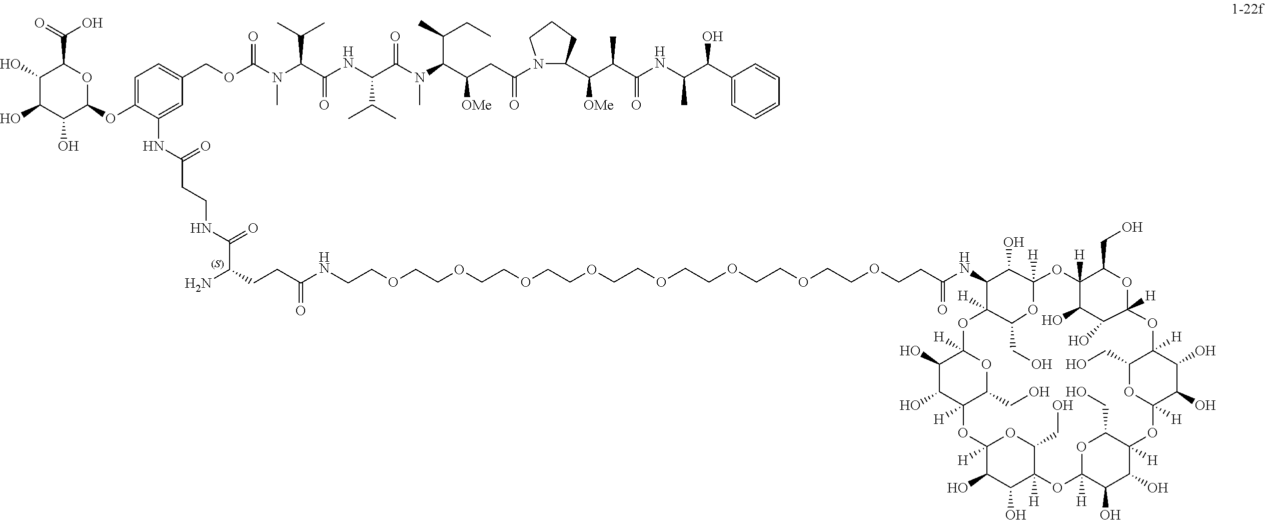

To the solution of 1-22e (12 mg, 0.004 mmol) in dry DMF (1 mL) was added Et₂NH (0.013 mL, 0.13 mmol), stirred at r.t. for 30 min. The resulting solution was concentrated to give 1-22f (11 mg, 99.4% yield) crude product, which was directly used in the next step. MS (ESI) m/z: 1319.4 [M/2+H]⁺

### Step 7: Synthesis of Compound 1-22g

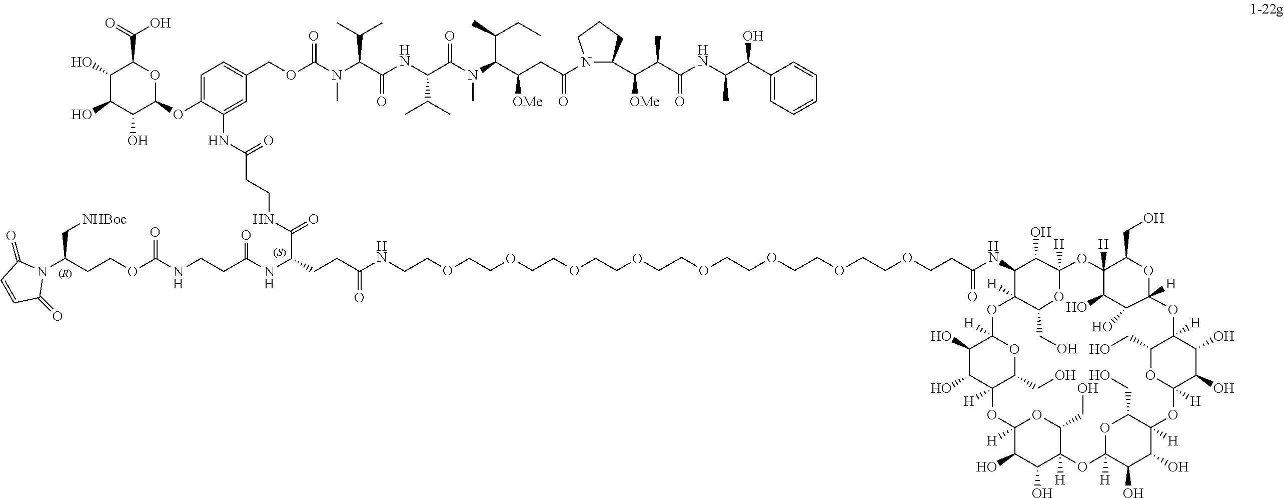

To the solution of Int-3 (2.8 mg, 0.007 mmol) in dry DMF (0.5 mL) were added HATU (2.7 mg, 0.007 mmol) and DIPEA (0.002 mL, 0.013 mmol), stirred at r.t. for 10 min. To the above mixture was added 1-22f (11 mg, 0.006 mmol), stirred at r.t. for 10 min. The resulting solution was purified by prep-HPLC (Method: column XBridge Prep C18 OBD 5 μm 19*150 mm; Mobile phase: A-water (0.1% FA): B-acetonitrile; Flow rate: 20 mL/min, the fraction was lyophilized to give 1-22g (10 mg, 52.6% yield) as a white solid. MS (ESI) m/z: 1510.4 [M/2+H]⁺

### Step 8: Synthesis of Compound 1-22

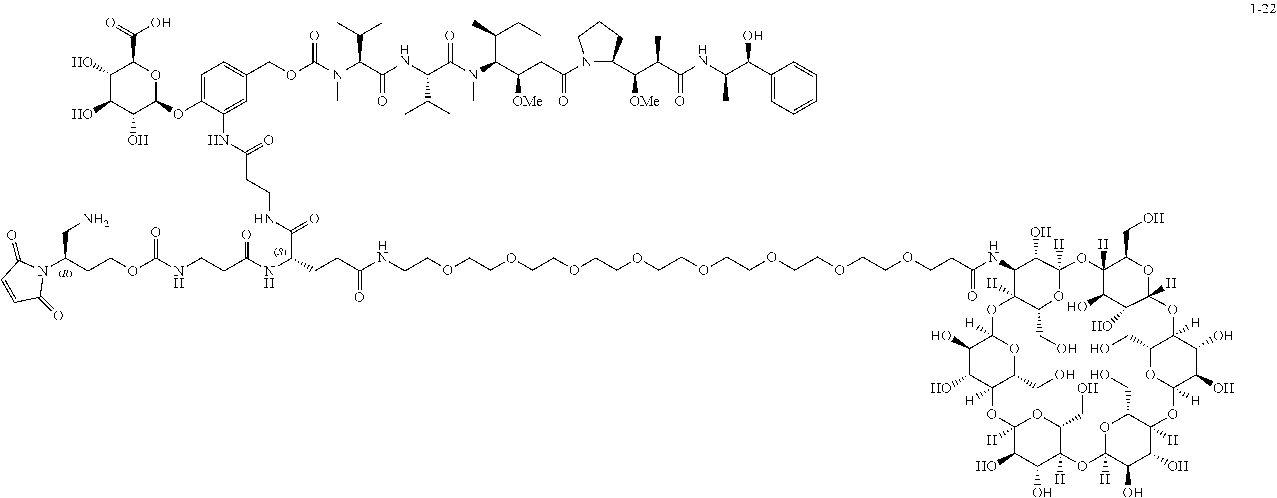

Compound 1-22 (6 mg, 62% yield) was synthesized according to the synthetic procedures of step 11 in example 1-2. MS (ESI) m/z: 1460.3 [M/2+H]⁺

### Example 1-23

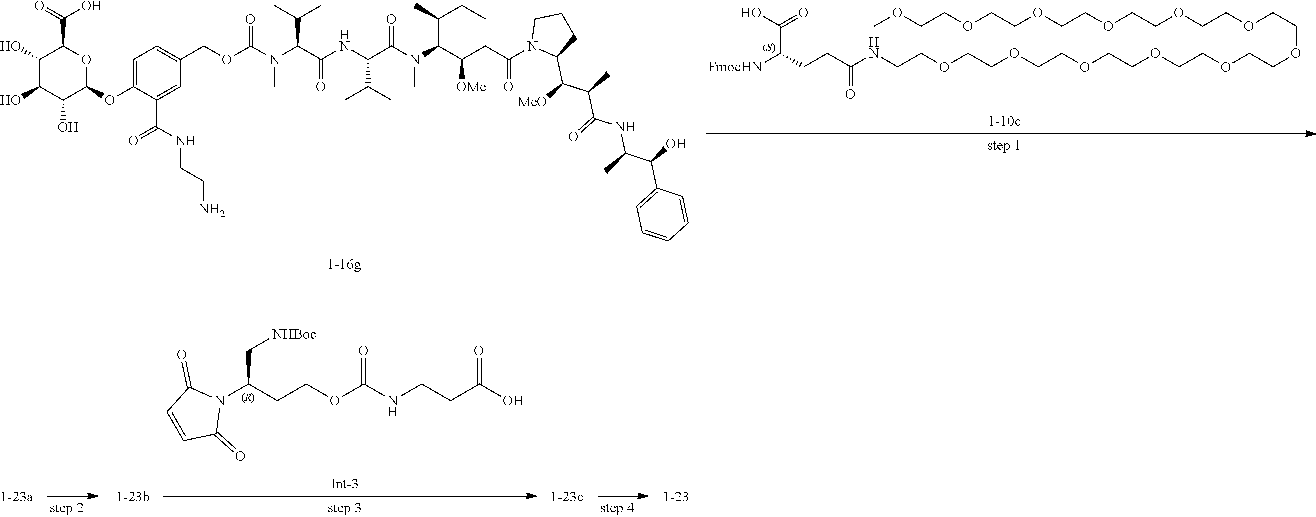

### Step 1: (2S,3S,4S,5R,6S)-6-(2-(((S)-42-((((9H-fluoren-9-yl)methoxy)carbonyl)amino)-39,43-dioxo-2,5,8,11,14,17,20,23,26,29,32,35-dodecaoxa-38,44-diazahexatetracontan-46-yl)carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (1-23a)

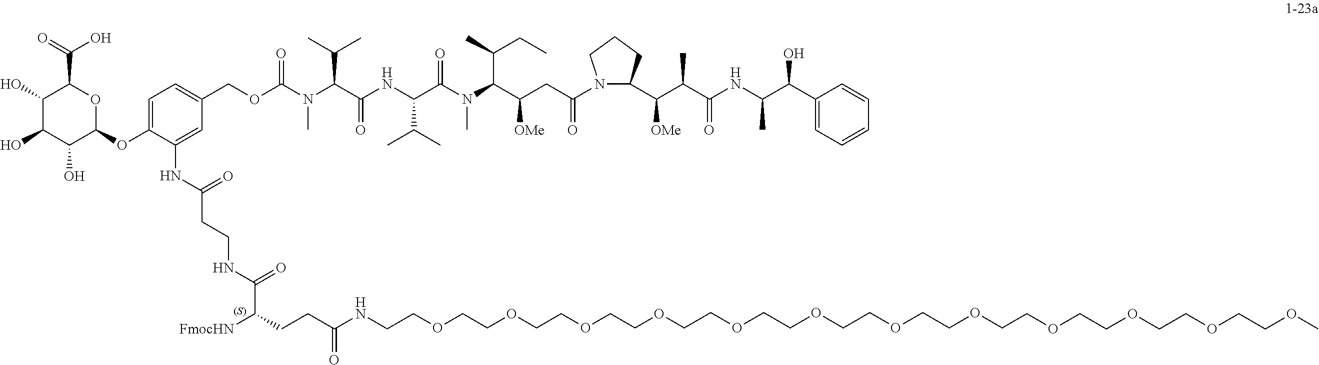

To the solution of 1-10c (111 mg, 0.12 mmol) in dry DMF (2 mL) were added HATU (46 mg, 0.12 mmol) and DIPEA (0.065 mL, 0.36 mmol), stirred at r.t. for 10 min. To the above mixture was added 1-16g (150 mg, 0.12 mmol), stirred at r.t. for 10 min. The resulting solution was purified by prep-HPLC (Method: column XBridge Prep C18 OBD 5 μm 19*150 mm; Mobile phase: A-water (0.1% FA): B-acetonitrile; Flow rate: 20 mL/min, the fraction was lyophilized to give 1-23a (150 mg, 61.5% yield) as a white solid. MS (ESI) m/z: 2024.9 [M+H]+

### Step 2: (2S,3S,4S,5R,6S)-6-(2-(((S)-42-amino-39,43-dioxo-2,5,8,11,14,17,20,23,26,29,32,35-dodecaoxa-38,44-diazahexatetracontan-46-yl)carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (1-23b)

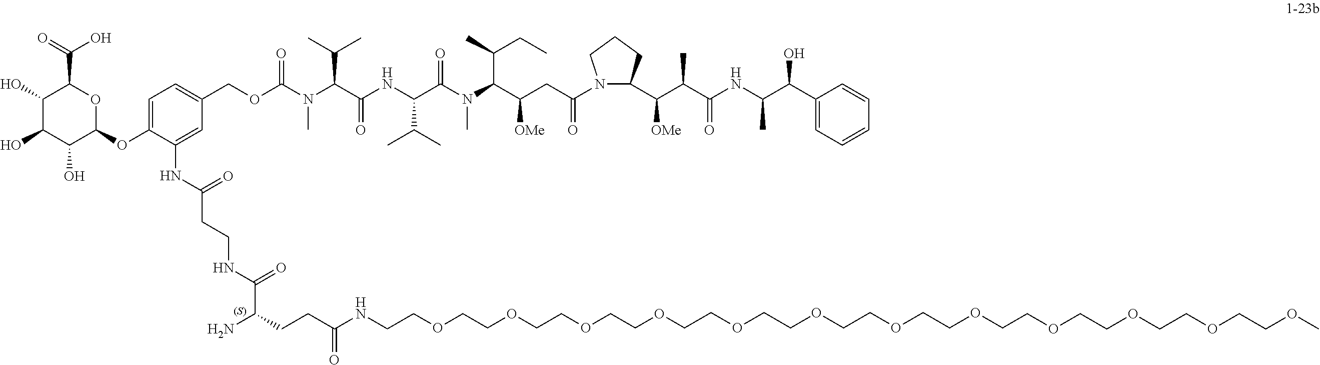

To the solution of 1-23a (150 mg, 0.074 mmol) in dry DMF (2 mL) was added Et₂NH (0.23 mL, 2.22 mmol), stirred at r.t. for 30 min. The resulting solution was concentrated to give 1-23b (125 mg, 93.6% yield) crude product, which was directly used in the next step. MS (ESI) m/z: 1802.5 [M+H]+

### Step 3: (2S,3S,4S,5R,6S)-6-(4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-(((S)-42-((R)-7-(2,5-dioxo-2,5-dihydro-1H-pyrrol-1-yl)-2,2-dimethyl-4,11-dioxo-3,10-dioxa-5,12-diazapentadecan-15-amido)-39,43-dioxo-2,5,8,11,14,17,20,23,26,29,32,35-dodecaoxa-38,44-diazahexatetracontan-46-yl)carbamoyl)phenoxy)-3,4,5-trihydroxytetrahydro-2H-pyran-2-carboxylic acid (1-23c)

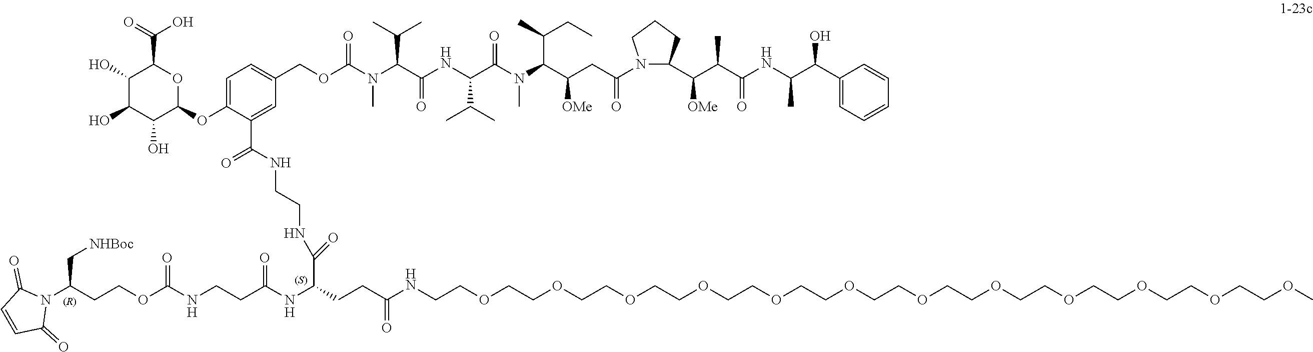

To the solution of Int-3 (28 mg, 0.069 mmol) in dry DMF (2 mL) were added HATU (26.7 mg, 0.069 mmol) and DIPEA (0.037 mL, 0.21 mmol), stirred at r.t. for 10 min. To the above mixture was added 1-23b (125 mg, 0.069 mmol), stirred at r.t. for 10 min. The resulting solution was purified by prep-HPLC (Method: column XBridge Prep C18 OBD 5 um 19*150 mm; Mobile phase: A-water (0.1% FA): B-acetonitrile; Flow rate: 20 mL/min, the fraction was lyophilized to give 1-23c (100 mg, 66% yield) as a white solid. MS (ESI) m/z: 2184.0 [M+H]⁺

### Step 4: (2S,3S,4S,5R,6S)-6-(2-(((S)-42-(3-((((R)-4-amino-3-(2,5-dioxo-2,5-dihydro-1H-pyrrol-1-yl) butoxy)carbonyl)amino)propanamido)-39,43-dioxo-2,5,8,11,14,17,20,23,26,29,32,35-dodecaoxa-38,44-diazahexatetracontan-46-yl)carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (1-23) trifluoroacetate

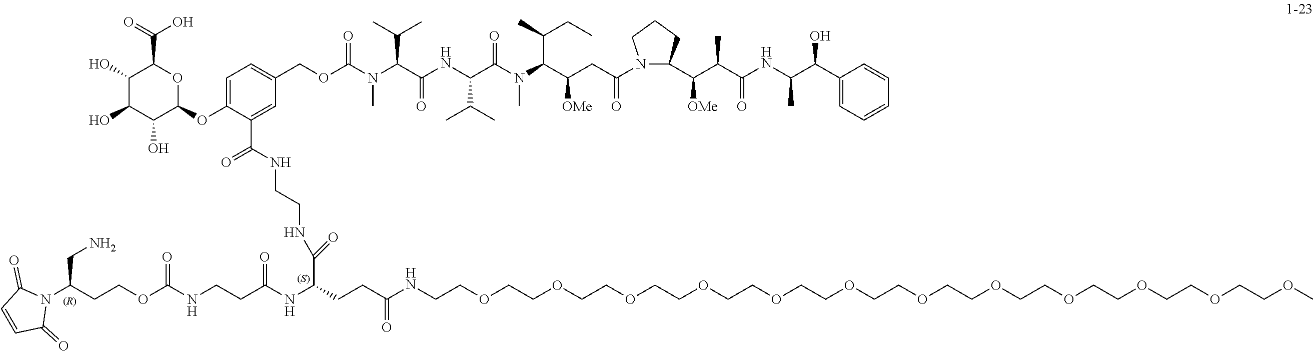

To the solution of 1-23c (100 mg, 0.046 mmol) in DCM (2 mL) was added TFA (0.38 mL, 4.93 mmol), stirred at 0° C. for 30 min. The resulting solution was concentrated to remove solvent. The residue was purified by prep-HPLC (Method: column XBridge Prep C18 OBD 5 um 19*150 mm; Mobile phase: A-water (0.05% TFA): B-acetonitrile; Flow rate: 20 mL/min, the fraction was lyophilized to give 1-23 (trifluoroacetate) (49 mg, 48.7% yield) as a white solid. MS (ESI) m/z: 2083.3 [M+H]⁺

### Example 1-24

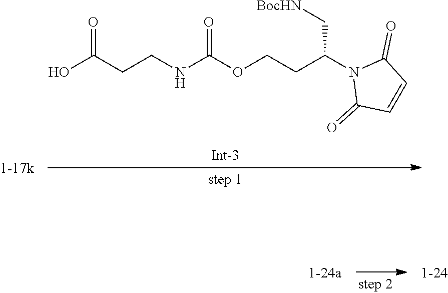 [SMILES: CC(C)(C)OC(=O)NC[C@@H](CCOC(=O)NCCC(=O)O)N1C(=O)C=CC1=O]

### Step 1: (2S,3S,4S,5R,6S)-6-(4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-((R,E)-16-(2,5-dioxopyrrolidin-1-yl)-21,21-dimethyl-8,12,19-trioxo-4,13,20-trioxa-3,7,11,18-tetraazadocos-2-en-2-yl)phenoxy)-3,4,5-trihydroxytetrahydro-2H-pyran-2-carboxylic acid 1-24a

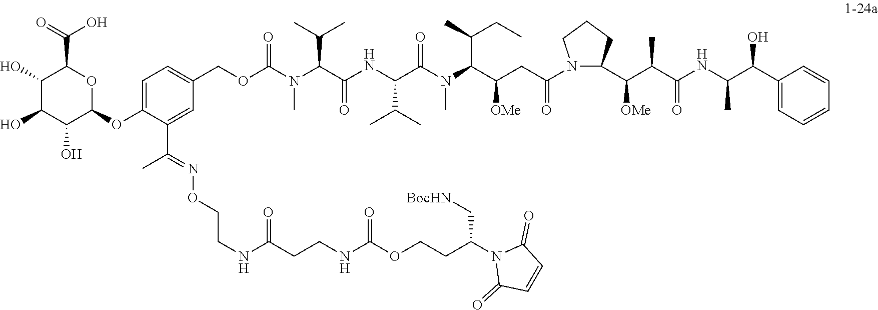 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2O[C@H](C(=O)O)[C@@H](O)[C@H](O)[C@H]2O)c(/C(C)=N/OCCNC(=O)CCNC(=O)OCC[C@H](CNC(=O)OC(C)(C)C)N2C(=O)C=CC2=O)c1)C(C)C]

To the solution of Int-3 (10.5 mg, 0.026 mmol) in dry DMF (1 mL) were added HATU (10 mg, 0.026 mmol) and DIPEA (9 μL, 0.053 mmol), stirred at r.t. for 10 min. Then to the above mixture was added 1-17k (20 mg, 0.0175 mmol), stirred at r.t. for 10 min. The resulting solution was purified by prep-HPLC (Method: column XBridge Prep C18 OBD 5 μm 19*150 mm; Mobile phase: A-water (0.1% TFA): B-acetonitrile; Flow rate: 20 mL/min, the fraction was lyophilized to give 1-24a (14 mg, 52.4% yield) as a white solid. MS (ESI) m/z: 1526.1 [M+H]

### Step 2: (2S,3S,4S,5R,6S)-6-(2-((R,E)-17-amino-16-(2,5-dioxopyrrolidin-1-yl)-8,12-dioxo-4,13-dioxa-3,7,11-triazaheptadec-2-en-2-yl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid 1-24

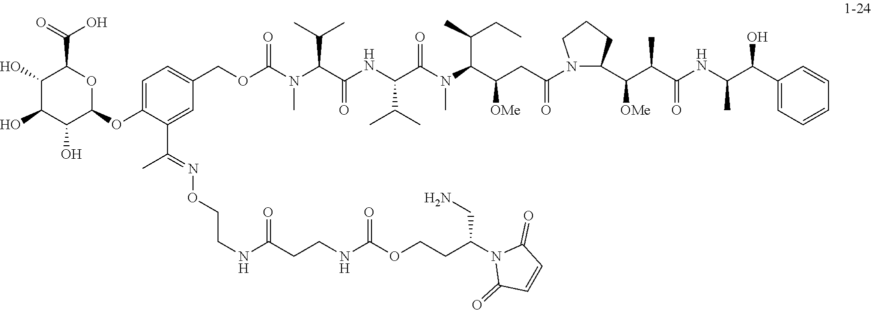 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2O[C@H](C(=O)O)[C@@H](O)[C@H](O)[C@H]2O)c(/C(C)=N/OCCNC(=O)CCNC(=O)OCC[C@H](CN)N2C(=O)C=CC2=O)c1)C(C)C]

To a solution of crude compound 1-24a (14 mg, 0.0091 mmol) in DCM (1.0 mL) was added TFA (0.2 mL) at 0° C. The mixture was stirred at r.t. for 10 min. The reaction was purified by prep-HPLC (Method: column XBridge Prep C18 OBD 5 μm 19*150 mm; Mobile phase: A-water (0.1% TFA): B-acetonitrile; Flow rate: 20 mL/min, the fraction was lyophilized to give 1-24 (trifluoroacetate) (7 mg, 53.9% yield) as a white solid. MS (ESI) m/z: 1426.8 [M+H]⁺.

### Example 1-25

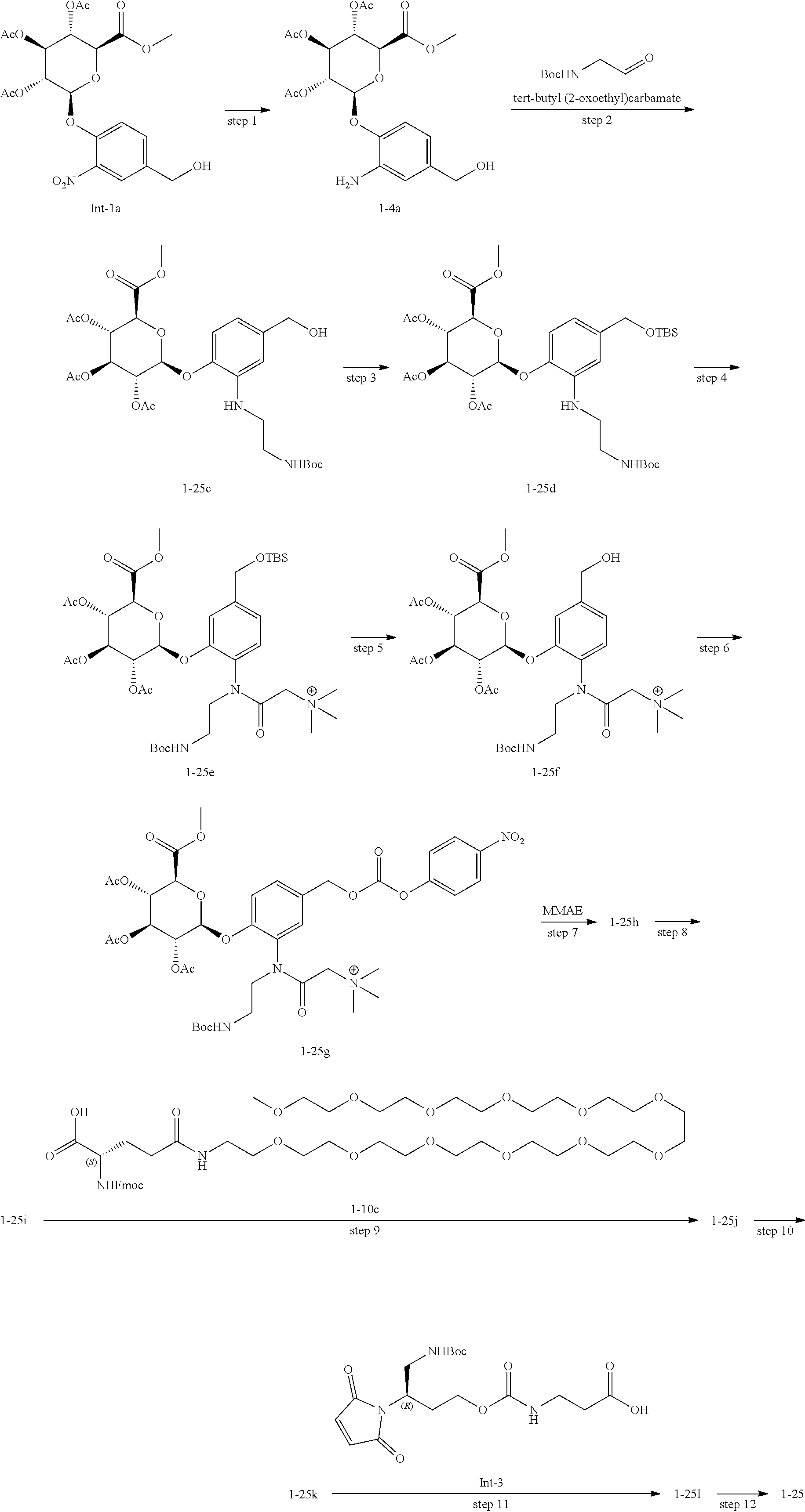

### Step 1: (2S,3R,4S,5S,6S)-2-(2-amino-4-(hydroxymethyl)phenoxy)-6-(methoxycarbonyl)tetrahydro-2H-pyran-3,4,5-triyl triacetate (1-4a)

To a solution of Int-1a (2.00 g, 4.1 mmol) in 10 mL THF was added PtO₂ (22.5 mg, 15% w/w). The mixture was stirred at r.t. for 2 h under H₂ atmosphere. The suspension was filtered through Celite, the filtrate was concentrated without further purification to give 1-4a as a colorless oil. MS (ESI) m/z: 456.6 [M+H]⁺

### Step 2: (2S,3R,4S,5S,6S)-2-(2-((2-((tert-butoxycarbonyl)amino)ethyl)amino)-4-(hydroxymethyl)phenoxy)-6-(methoxycarbonyl)tetrahydro-2H-pyran-3,4,5-triyl triacetate (1-25c)

To a solution of 1-4a and tert-butyl (2-oxoethyl)carbamate (1.31 g, 8.3 mmol) in 10 mL MeOH was added NaCNBH₃ (778 mg, 12.4 mmol). The mixture was stirred at r.t. for 3 h. The mixture was partitioned between water and ethyl acetate. The organic layer was concentrated and purified by flash column chromatography (Petroleum ether/EtOAc=50/50) to give 1-25c (1.68 g, 68.0% yield in two steps) as a white solid. MS (ESI) m/z: 599.6 [M+H]:

### Step 3: (2S,3R,4S,5S,6S)-2-(2-((2-((tert-butoxycarbonyl)amino)ethyl)amino)-4-(((tert-butyldimethylsilyl)oxy)methyl)phenoxy)-6-(methoxycarbonyl)tetrahydro-2H-pyran-3,4,5-triyl triacetate (1-25d)

To a solution of 1-25c (1.68 g, 2.8 mmol), imidazole (191 mg, 2.8 mmol) and DMAP (86 mg, 0.7 mmol) in 4 mL DMF was added TBSCl (423 mg, 2.8 mmol). The mixture was stirred at r.t. for 3 h. The mixture was partitioned between water and ethyl acetate. The organic layer was concentrated and purified by flash column chromatography (PE/EA=60/40) to give 1-25d (1.51 g, 75.5% yield) as a white solid. MS (ESI) m/z: 713.8 [M+H]⁺

### Step 4: 2-((2-((tert-butoxycarbonyl)amino)ethyl) (4-(((tert-butyldimethylsilyl)oxy)methyl)-2-(((2S,3R,4S,5S,6S)-3,4,5-triacetoxy-6-(methoxycarbonyl)tetrahydro-2H-pyran-2-yl)oxy)phenyl)amino)-N,N,N-trimethyl-2-oxoethan-1-aminium (1-25e)

To a solution of 1-25d (1.00, 1.4 mmol), betaine (237 mg, 1.5 mmol), pyridine (2.3 mL, 28.0 mmol) in 10 mL CH₃CN was added POCl₃ (144 μL, 1.5 mmol) at 0° C. The mixture was stirred at r.t. for 1 h. The mixture was quenched with 5 mL H₂O and concentrated to give crude 1-25e as a white solid. MS (ESI) m/z: 814.0 [M+H]⁺

### Step 5: 2-((2-((tert-butoxycarbonyl)amino)ethyl) (4-(hydroxymethyl)-2-(((2S,3R,4S,5S,6S)-3,4,5-triacetoxy-6-(methoxycarbonyl)tetrahydro-2H-pyran-2-yl)oxy)phenyl)amino)-N,N,N-trimethyl-2-oxoethan-1-aminium (1-25f)

Crude 1-25e was dissolved in AcOH/THF/H₂O (6/2/2 mL) and stirred at r.t. for 30 min. The mixture was purified by prep-HPLC (Column: Xbridge Prep C18 OBD™ 5 μm, 19*150 mm; Mobile phase A: 0.1% FA in water, B: MeCN; Gradient: 25%-60% B; Flow rate: 20 mL/min), the fraction was lyophilized to give 1-25f (500 mg, 51.0% yield in two steps) as a white solid. MS (ESI) m/z: 698.8 [M+H]⁺

### Step 6: 2-((2-((tert-butoxycarbonyl)amino)ethyl) (5-((((4-nitrophenoxy)carbonyl)oxy)methyl)-2-(((2S,3R,4S,5S,6S)-3,4,5-triacetoxy-6-(methoxycarbonyl)tetrahydro-2H-pyran-2-yl)oxy)phenyl)amino)-N,N,N-trimethyl-2-oxoethan-1-aminium (1-25g)

To a solution of 1-25f (250 mg, 0.36 mmol) and bis(4-nitrophenyl) carbonate (125 mg, 0.41 mmol) in dry CH₂Cl (3 mL) was added DIPEA (125 μL, 0.72 mmol). The mixture was stirred at r.t. for 3 h and purified by prep-HPLC (Column: Xbridge Prep C18 OBD™ 5 μm, 19*150 mm; Mobile phase A: 0.1% FA in water, B: MeCN; Gradient: 40%-60% B: Flow rate: 20 mL/min), the fraction was lyophilized to give 1-25g (145 mg, 46.9% yield) as a white solid. MS (ESI) m/z: 863.9 [M+H]⁺

### Step 7: 2-((2-((tert-butoxycarbonyl)amino)ethyl) (5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-(((2S,3R,4S,5S,6S)-3,4,5-triacetoxy-6-(methoxycarbonyl)tetrahydro-2H-pyran-2-yl)oxy)phenyl)amino)-N,N,N-trimethyl-2-oxoethan-1-aminium (1-25h)

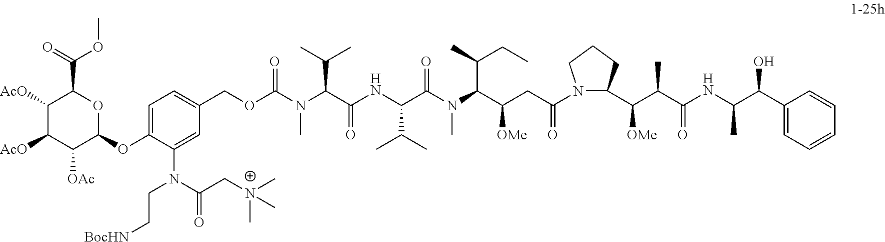 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2O[C@H](C(=O)OC)[C@@H](OC(C)=O)[C@H](OC(C)=O)[C@H]2OC(C)=O)c(N(CCNC(=O)OC(C)(C)C)C(=O)C[N+](C)(C)C)c1)C(C)C]

To a solution of 1-25g (145 mg, 0.17 mmol) in 4 mL DMF was added HOBT (5.7 mg, 0.042 mmol) and DIPEA (58 μL, 0.34 mmol), followed by addition of MMAE (121 mg, 0.17 mmol). The mixture was stirred at r.t. for 24 h and purified by prep-HPLC (Column: Xbridge Prep C18 OBD™ 5 μm, 19*150 mm; Mobile phase A: 0.1% FA in water, B: MeCN; Gradient: 25%-65% B; Flow rate: 20 mL/min), the fraction was lyophilized to give 1-25h (180 mg, 74.3% yield) as a white solid. MS (ESI) m/z: 1442.7 [M+H]⁺

### Step 8: 2-((2-aminoethyl) (5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-(((2S,3R,4S,5S,6S)-3,4,5-triacetoxy-6-(methoxycarbonyl)tetrahydro-2H-pyran-2-yl)oxy)phenyl)amino)-N,N,N-trimethyl-2-oxoethan-1-aminium (1-25i)

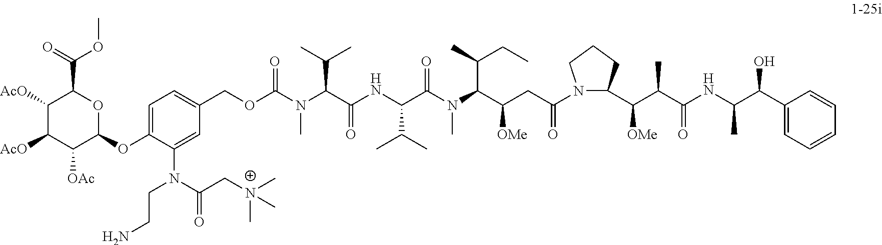 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2O[C@H](C(=O)OC)[C@@H](OC(C)=O)[C@H](OC(C)=O)[C@H]2OC(C)=O)c(N(CCN)C(=O)C[N+](C)(C)C)c1)C(C)C]

1-25h (100 mg, 0.069 mmol) was dissolved in dry CH₂Cl₂ (1 mL), 300 μL HCl (4 M in 1,4-Dioxane) was added. The mixture was stirred for 30 min at r.t. and was concentrated without further purification to give crude 1-25i as a white solid. MS (ESI) m/z: 1342.8 [M+H]⁺

### Step 9: (S)-42-((((9H-fluoren-9-yl)methoxy)carbonyl)amino)-47-(5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-(((2S,3R,4S,5S,6S)-3,4,5-triacetoxy-6-(methoxycarbonyl)tetrahydro-2H-pyran-2-yl)oxy)phenyl)-N,N,N-trimethyl-39,43,48-trioxo-2,5,8,11,14,17,20,23,26,29,32,35-dodecaoxa-38,44,47-triazanonatetracontan-49-aminium (1-25j)

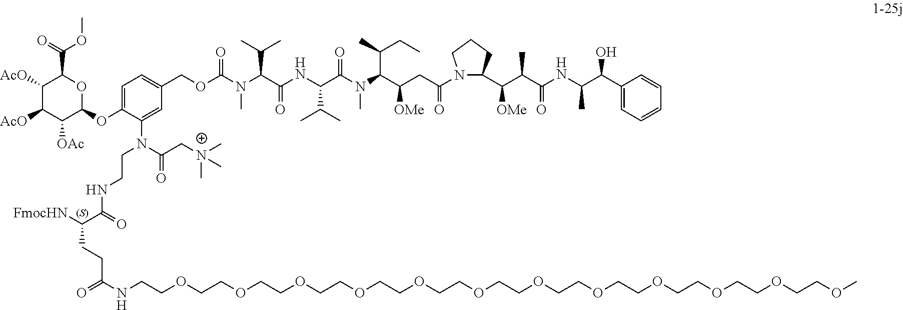

The crude 1-25i was dissolved in dry DMF (1 mL). 1-10c (65 mg, 0.070 mmol), HATU (32 mg, 0.084 mmol) and DIPEA (37 μL, 0.21 mmol) were added to the mixture. The mixture was stirred for 3 h at r.t. and purified by prep-HPLC (Column: Xbridge Prep C18 OBD™ 5 μm, 19*150 mm; Mobile phase A: 0.1% FA in water. B: MeCN; Gradient: 25%-60% B: Flow rate: 20 mL/min), the fraction was lyophilized to give 1-25j (78 mg, 49.7% yield in two steps) as a white solid. MS (ESI) m/z: 2235.8 [M+H]⁺

### Step 10: (S)-42-amino-47-(5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-(((2S,3R,4S,5S,6S)-6-carboxy-3,4,5-trihydroxytetrahydro-2H-pyran-2-yl)oxy)phenyl)-N,N,N-trimethyl-39,43,48-trioxo-2,5,8,11,14,17,20,23,26,29,32,35-dodecaoxa-38,44,47-triazanonatetracontan-49-aminium (1-25k)

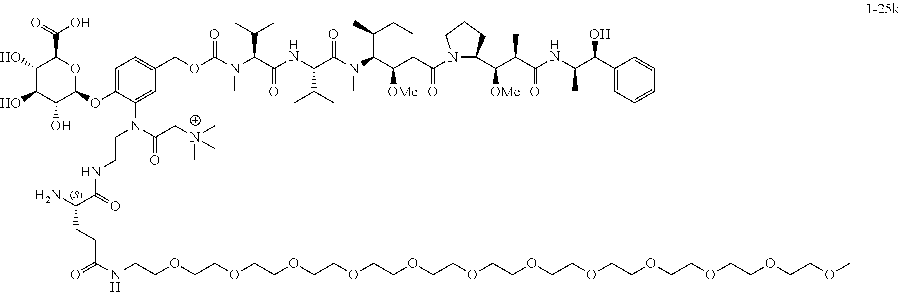

To a solution of 1-25j in THF (2 mL) and MeOH (2 mL) was added 1N LiOH (209 μL) at 0° C. The mixture was stirred at r.t. for 15 min and quenched with 200 μL AcOH. The crude product was purified by prep-HPLC (Column: Sunfire Prep C18 OBD™ 5 μm, 19*150 mm; Mobile phase A: 0.1% FA in water, B: MeCN; Gradient: 25%-55% B; Flow rate: 20 mL/min), the fraction was lyophilized to give 1-25k (50 mg, 76.5% yield) as a white solid. MS (ESI) m/z: 1873.7 [M+H]⁺

### Step 11: (S)-47-(5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-(((2S,3R,4S,5S,6S)-6-carboxy-3,4,5-trihydroxytetrahydro-2H-pyran-2-yl)oxy)phenyl)-42-((R)-7-(2,5-dioxo-2,5-dihydro-1H-pyrrol-1-yl)-2,2-dimethyl-4,11-dioxo-3,10-dioxa-5,12-diazapentadecan-15-amido)-N,N,N-trimethyl-39,43,48-trioxo-2,5,8,11,14,17,20,23,26,29,32,35-dodecaoxa-38,44,47-triazanonatetracontan-49-aminium (1-25l)

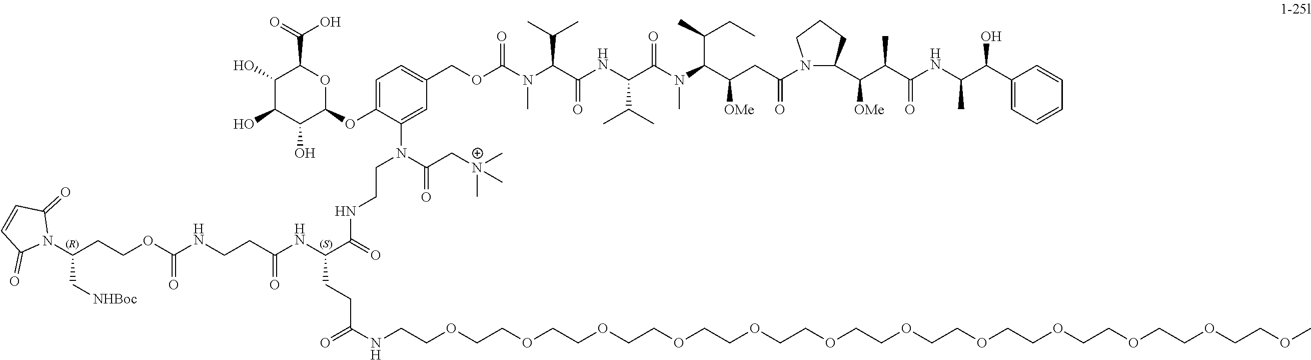

Compound 1-25l (40 mg, 70.9% yield) was synthesized according to the synthetic procedures of step 10 in example 1-20. MS (ESI) m/z: 2255.3 [M+H]⁺

### Step 12: (S)-42-(3-((((R)-4-amino-3-(2,5-dioxo-2,5-dihydro-1H-pyrrol-1-yl) butoxy)carbonyl)amino)propanamido)-47-(5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-(((2S,3R,4S,5S,6S)-6-carboxy-3,4,5-trihydroxytetrahydro-2H-pyran-2-yl)oxy)phenyl)-N,N,N-trimethyl-39,43,48-trioxo-2,5,8,11,14,17,20,23,26,29,32,35-dodecaoxa-38,44,47-triazanonatetracontan-49-aminium (1-25)

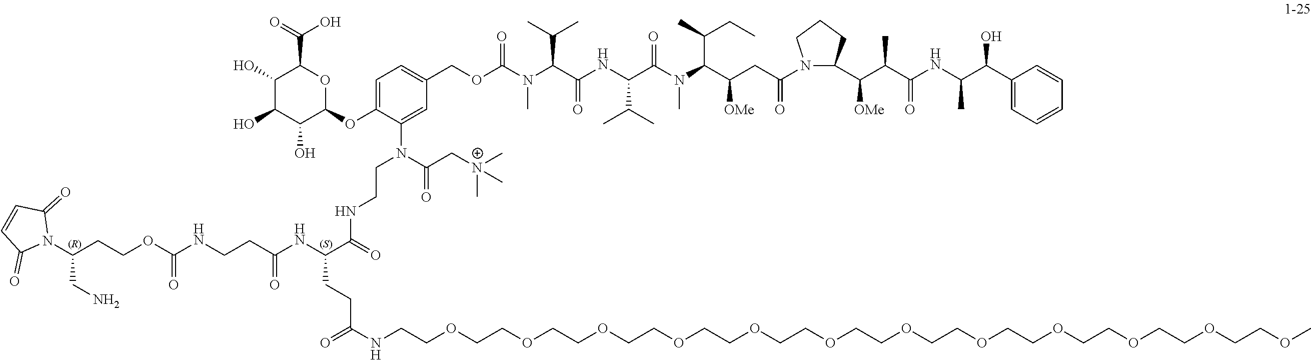

Compound 1-25 (19 mg, 49.7% yield) was synthesized according to the synthetic procedures of step 11 in example 1-20. MS (ESI) m/z: 2155.0 [M+H]⁺

### Example 1-26

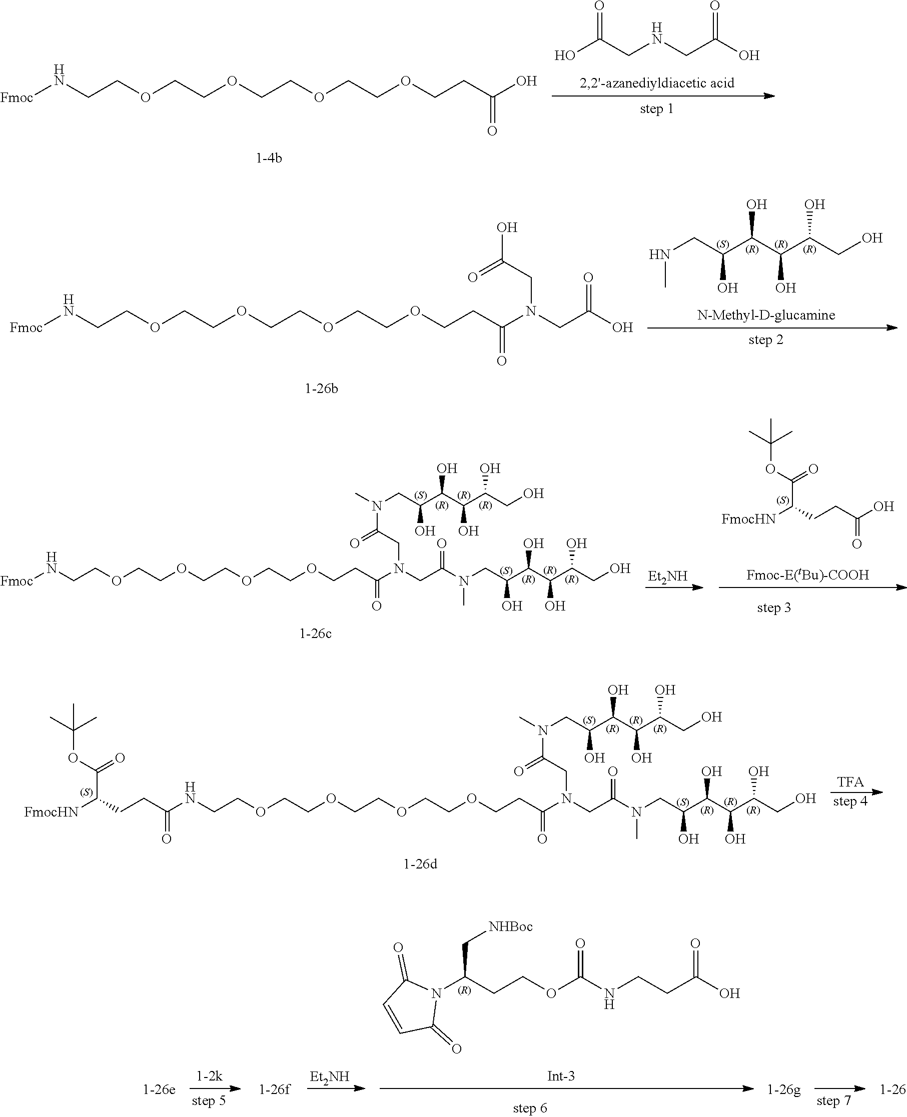

### Step 1: 20-(carboxymethyl)-1-(9H-fluoren-9-yl)-3,19-dioxo-2,7,10,13,16-pentaoxa-4,20-diazadocosan-22-oic acid (1-26b)

To a solution of 1-4b (680 mg, 1.4 mmol), HATU (583 mg, 1.5 mmol), DIPEA (486 μL, 2.8 mmol) in 10 mL dry DMF was added 2,2′-azanediyldiacetic acid (371 mg, 2.8 mmol). The mixture was stirred at r.t. overnight. The mixture was partitioned between water and ethyl acetate. The organic layer was concentrated and purified by flash column chromatography (CH₂Cl/MeOH=96/4) to give 1-26b (250 mg, 19.7% yield) as a white solid. MS (ESI) m/z: 603.2 [M+H]⁺

### Step 2: (9H-fluoren-9-yl)methyl((21S,22R,23R,24R)-21,22,23,24,25-pentahydroxy-19-methyl-16-(2-(methyl((2S,3R,4R,5R)-2,3,4,5,6-pentahydroxyhexyl)amino)-2-oxoethyl)-15,18-dioxo-3,6,9,12-tetraoxa-16,19-diazapentacosyl)carbamate (1-26c)

To a solution of 1-26b (250 mg, 0.41 mmol), HATU (315 mg, 0.83 mmol), DIPEA (217 UL, 1.2 mmol) in 10 mL dry DMF was added N-Methyl-D-glucamine (162 mg, 0.83 mmol). The mixture was stirred at r.t. for 4 h and purified by prep-HPLC (Column: Xbridge Prep C18 OBD™ 5 μm, 19*150 mm; Mobile phase A: 0.1% FA in water, B: MeCN; Gradient: 25%-60% B: Flow rate: 20 mL/min), the fraction was lyophilized to give 1-26c (246 mg, 62.0% yield) as a white solid. MS (ESI) m/z: 957.7 [M+H]⁺

### Step 3: tert-butyl (2S,27S,28R,29R,30R)-2-((((9H-fluoren-9-yl)methoxy)carbonyl)amino)-27,28,29,30,31-pentahydroxy-25-methyl-22-(2-(methyl((2S,3R,4R,5R)-2,3,4,5,6-pentahydroxyhexyl)amino)-2-oxoethyl)-5,21,24-trioxo-9,12,15,18-tetraoxa-6,22,25-triazahentriacontanoate (1-26d)

To a solution of 1-26c (246 mg, 0.26 mmol) in 3 mL DMF was added Et₂NH (266 μL, 2.6 mmol). The mixture was stirred at r.t. for 30 min and was concentrated to give crude product. To a solution of Fmoc-E (^{t}Bu)-COOH (125 mg, 0.29 mmol), HATU (134 mg, 0.35 mmol) and DIPEA (102 μL, 0.59 mmol) in 2 mL DMF was added the crude product. The mixture was stirred at r.t. for 1 h and was purified by prep-HPLC (Column: Xbridge Prep C18 OBD™ 5 μm, 19*150 mm; Mobile phase A: 0.1% FA in water, B: MeCN; Gradient: 25%-60% B; Flow rate: 20 mL/min), the fraction was lyophilized to give 1-26d (200 mg, 59.6% yield) as a white solid. MS (ESI) m/z: 1143.0 [M+H]⁺

### Step 4: (2S,27S,28R,29R,30R)-2-((((9H-fluoren-9-yl)methoxy)carbonyl)amino)-27,28,29,30,31-pentahydroxy-25-methyl-22-(2-(methyl((2S,3R,4R,5R)-2,3,4,5,6-pentahydroxyhexyl)amino)-2-oxoethyl)-5,21,24-trioxo-9,12,15,18-tetraoxa-6,22,25-triazahentriacontanoic acid (1-26e)

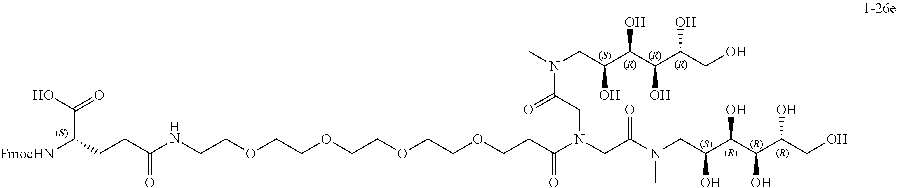

To a solution of 1-26d in CH₂Cl₂ (2 mL) was added TFA (600 μL) at 0° C. The mixture was stirred at r.t. for 3 h, then purified by prep-HPLC (Column: Xbridge Prep C18 OBD™ 5 μm, 19*150 mm; Mobile phase A: 0.1% FA in water, B: MeCN; Gradient: 25%-55% B; Flow rate: 20 mL/min), the fraction was lyophilized to give 1-26e (123 mg, 64.7% yield) as a white solid. MS (ESI) m/z: 1086.7 [M+H]⁺

### Step 5: (2S,3S,4S,5R,6S)-6-(2-((6S,31S,32R,33R,34R)-6-((((9H-fluoren-9-yl)methoxy)carbonyl)amino)-31,32,33,34,35-pentahydroxy-29-methyl-26-(2-(methyl((2S,3R,4R,5R)-2,3,4,5,6-pentahydroxyhexyl)amino)-2-oxoethyl)-5,9,25,28-tetraoxo-13,16,19,22-tetraoxa-4,10,26,29-tetraazapentatriacontanamido)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (1-26f)

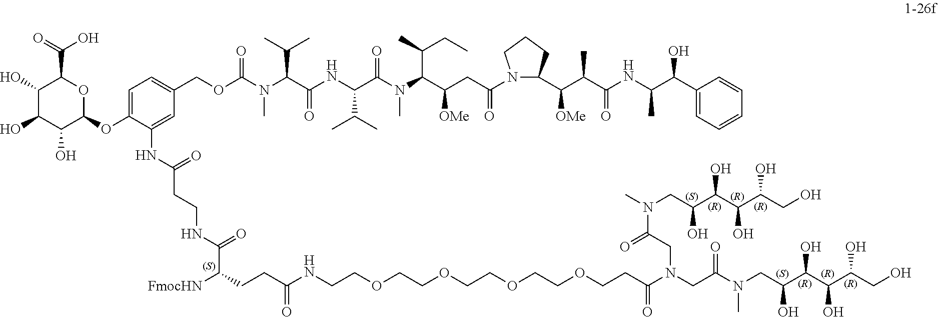

Compound 1-26f (125 mg, 50.2% yield) was synthesized according to the synthetic procedures of step 8 in example 1-2. MS (ESI) m/z: 2199.5 [M+H]⁺

### Step 6: (2S,3S,4S,5R,6S)-6-(4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)-2-((6S,31S,32R,33R,34R)-6-((R)-7-(2,5-dioxo-2,5-dihydro-1H-pyrrol-1-yl)-2,2-dimethyl-4,11-dioxo-3,10-dioxa-5,12-diazapentadecan-15-amido)-31,32,33,34,35-pentahydroxy-29-methyl-26-(2-(methyl((2S,3R,4R,5R)-2,3,4,5,6-pentahydroxyhexyl)amino)-2-oxoethyl)-5,9,25,28-tetraoxo-13,16,19,22-tetraoxa-4,10,26,29-tetraazapentatriacontanamido)phenoxy)-3,4,5-trihydroxytetrahydro-2H-pyran-2-carboxylic acid (1-26g)

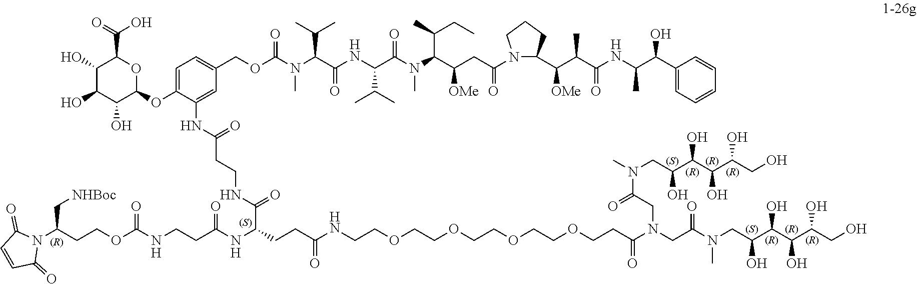

Compound 1-26g (40 mg, 67.8% yield) was synthesized according to the synthetic procedures of step 9-10 in example 1-2. MS (ESI) m/z: 1179.6 [M+2H⁺]/2

### Step 7: (2S,3S,4S,5R,6S)-6-(2-((6S,31S,32R,33R,34R)-6-(3-((((R)-4-amino-3-(2,5-dioxo-2,5-dihydro-1H-pyrrol-1-yl) butoxy)carbonyl)amino)propanamido)-31,32,33,34,35-pentahydroxy-29-methyl-26-(2-(methyl((2S,3R,4R,5R)-2,3,4,5,6-pentahydroxyhexyl)amino)-2-oxoethyl)-5,9,25,28-tetraoxo-13,16,19,22-tetraoxa-4,10,26,29-tetraazapentatriacontanamido)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (1-26)

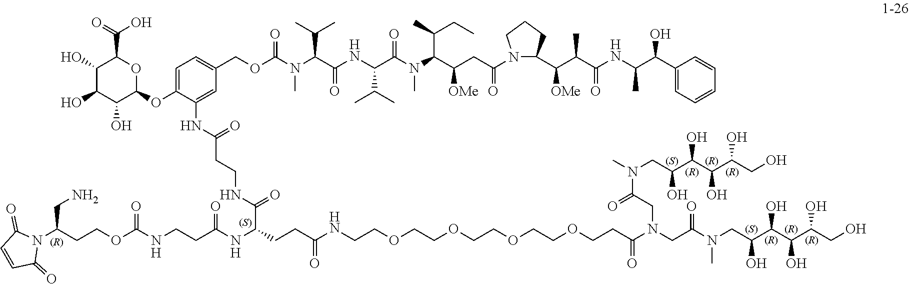

Compound 1-26 (20.8 mg, 54.3% yield) was synthesized according to the synthetic procedures of step 11 in example 1-2. MS (ESI) m/z: 2258.5 [M+H]⁺.

Conjugator-linker-payload compounds 1-1 to 1-26 are summarized below in Table 1.

### Example 2: Antibody Drug Conjugate Preparation and Characterization

DAR 3-5 antibody drug conjugate preparation. An antibody in a conjugation buffer (with concentration 0.5-25 mg/mL, PBS buffer pH 6.0-8.5) was incubated under reduction temperature (0-40° C.) for 10 min. 2-10 eq. TECP solution (5 mM stock in PBS buffer) was added to the reaction mixture and the reduction reaction was left for 1-8 hours at reduction temperature. Organic solvent (e.g., DMSO, DMF, DMA, PG, acetonitrile, 0-25% v/v) and the conjugator-linker-payload stock prepared herein (6-25 eq, 10 mM stock in organic solvent) (see Tables 2A-2D) were added stepwise after the reduction mixture was cooled to 0-25° C. Conjugation solution was left for 1-3 h at 0-25° C., and the reaction can be quenched with N-acetyl cysteine (1 mM stock). The solution was submitted to buffer exchange (spin desalting column, ultrafiltration, and dialysis) into storage buffer (for example, pH 5.5-6.5 histidine acetate buffer, with optional additive such as sucrose, trehalose, tween 20, 60, 80).

After the conjugation step, the ADC was subjected to buffer exchange into ring opening buffer (pH 6.5˜9.0. PBS, borate or tris buffer) and the solution was left at 22 or 37° C. for 1˜48 h. The maleimide ring opening process was monitored via reduced LCMS. Once the conjugated maleimide hydrolysis was completed, the resulting ADCs were buffer exchanged into basic tris pH 8.0-8.5 buffer or acidic histidine-acetate pH 5.0-6.5 buffer via dialysis.

LCMS method to monitor and determine maleimide hydrolysis. LC-MS analysis was carried out under the following measurement conditions:
LC-MS system: Vanquish Flex UHPLC and Orbitrap Exploris 240 Mass Spectrometer
Column: MAbPac™ RP, 2.1*50 mm, 4 μm, 1,500 Å. Thermo Scientific™
Column temperature: 80° C.
Mobile phase A: 0.1% formic acid (FA) aqueous solution
Mobile phase B: Acetonitrile solution containing 0.1% formic acid (FA)
Gradient program 1: 25% B-25% B (0 min-2 min), 25% B-50% B (2 min-18 min), 50% B-90% B (18 min-18.1 min), 90% B-90% B (18.1 min-20 min), 90% B-25% B (20 min-20.1 min), 25% B-25% B (20.1 min-25 min)
Gradient program 2
Injected sample amount: 2 μg
MS parameters: Intact and denaturing MS data were acquired in HMR mode at setting of R=15k and deconvolved using the ReSpect™ algorithm and Sliding Window integration in Thermo Scientific™ BioPharma Finder™ 4.0 software.

ADC characterization. ADCs were characterized using the following analytical methods. Drug to antibody ratios (DAR) of the ADCs were determined by LCMS method or HIC method. SEC purity of ADCs made were all >95% purity.

LCMS method: LC-MS analysis was carried out under the following measurement conditions:
LC-MS system: Vanquish Flex UHPLC and Orbitrap Exploris 240 Mass Spectrometer
Column: MAbPac™ RP, 2.1*50 mm, 4 μm, 1,500 Å, Thermo Scientific™
Column temperature: 80° C.
Mobile phase A: 0.1% formic acid (FA) aqueous solution
Mobile phase B: Acetonitrile solution containing 0.1% formic acid (FA)
Gradient program: 25% B-25% B (0 min-2 min), 25% B-50% B (2 min-18 min), 50% B-90% B (18 min-18.1 min), 90% B-90% B (18.1 min-20 min), 90% B-25% B (20 min-20.1 min), 25% B-25% B (20.1 min-25 min)
Injected sample amount: 1 μg
MS parameters: Intact and denaturing MS data were acquired in HMR mode at setting of
R=15k and deconvolved using the ReSpect™ algorithm and Sliding Window integration in Thermo Scientific™ BioPharma Finder™ 4.0 software.
HIC method: HPLC analysis was carried out under the following measurement conditions:
HPLC system: Waters ACQUITY ARC HPLC System
Detector: measurement wavelength: 280 nm
Column: Tosoh Bioscience 4.6 μm ID×3.5 cm, 2.5 μm butyl-nonporous resin column
Column temperature: 25° C.
Mobile phase A: 1.5 M ammonium sulfate, 50 mM phosphate buffer, pH 7.0
Mobile phase B: 50 mM phosphate buffer, 25% (V/V) Isopropanol, pH 7.0
Gradient program: 0% B-0% B (0 min-2 min), 0% B-100% B (2 min-15 min), 100% B-100% B (15 min-16 min), 100% B-0% B (16 min-17 min), 0% B-0% B (17 min-20 min)
Injected sample amount: 20 μg

SEC method to determine ADC purity: HPLC analysis was carried out under the following measurement conditions:
HPLC system: Waters H-Class UPLC System
Detector: measurement wavelength: 280 nm
Column: ACQUITY UPLC BEH200 SEC 1.7 um 4.6×150 mm, Waters
Column temperature: room temperature
Mobile phase A: 200 mM Phosphate buffer, 250 mM potassium chloride, 15% isopropyl alcohol, pH 7.0
Gradient program: under 10 min isocratic elutions with the flow rate of 0.3 mL/min
Injected sample amount: 20 μg

ADC hydrophobicity evaluation: An ADC with a higher hydrophobic property would appear with a later retention time (“RT”) from HIC (hydrophobicity interaction column) chromatography. Results are presented in Tables 2A-2D using the DAR8 peak as a reference.

TABLE 1
Conjugator-linker-payloads
Conjugator-Linker-Payload Structure and Compound No.

| | |
|---|---|
| 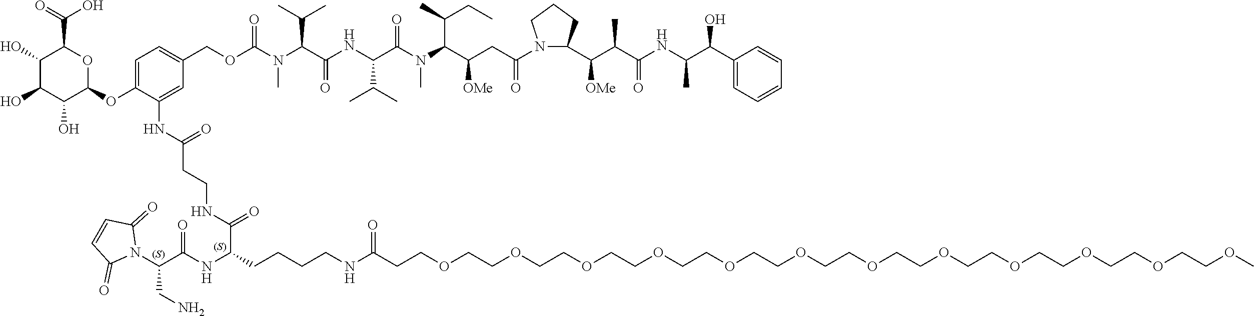 | 1-1 |
| 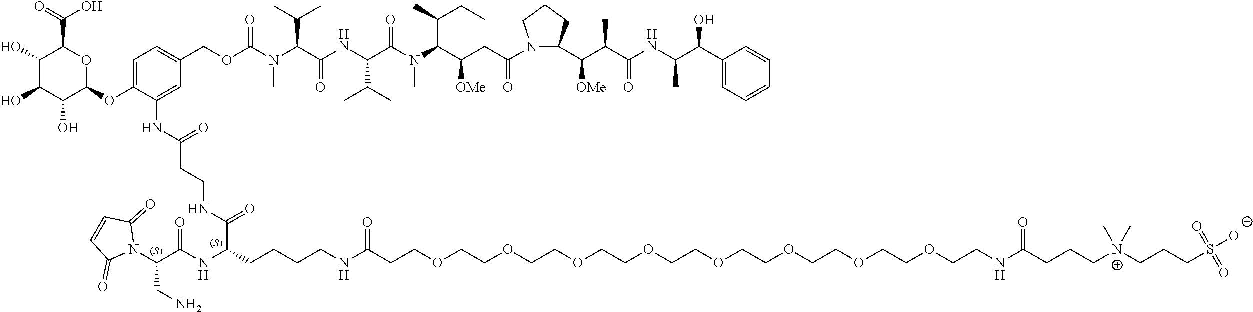 | 1-2 |
| 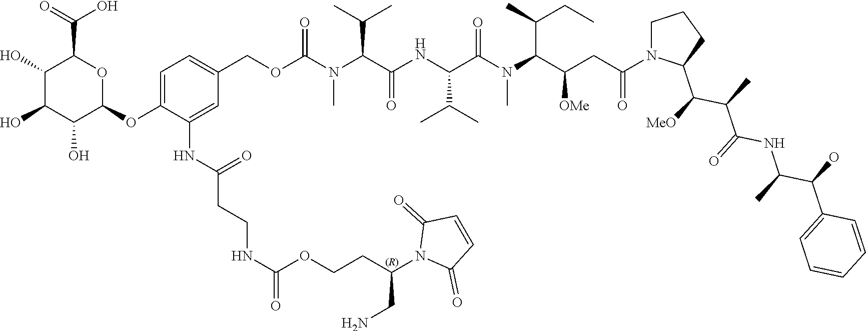 | 1-3 |
| 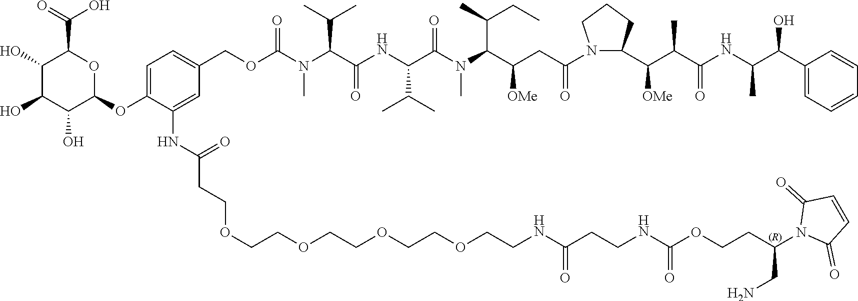 | 1-4 |
| 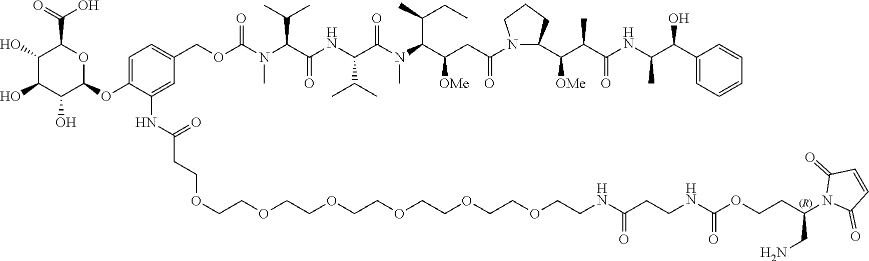 | 1-5 |
| 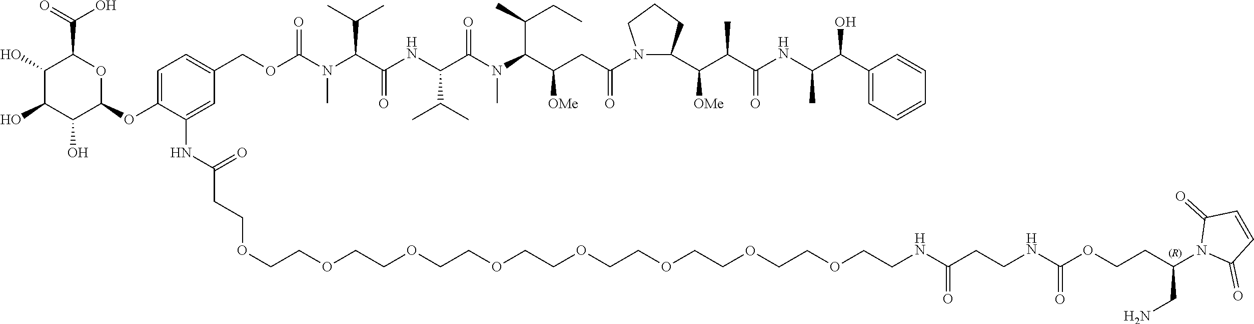 | 1-6 |
| 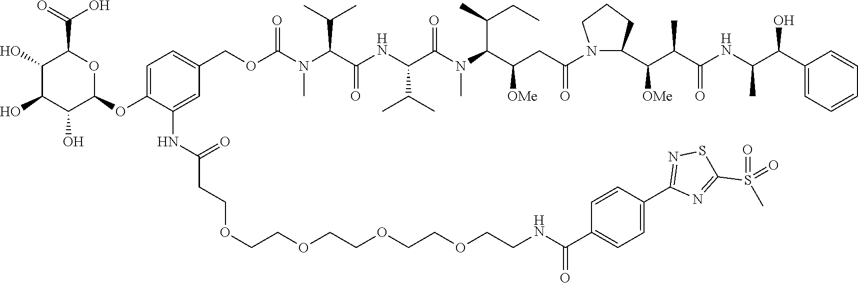 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2O[C@H](C(=O)O)[C@@H](O)[C@H](O)[C@H]2O)c(NC(=O)CCOCCOCCOCCOCCNC(=O)c2ccc(-c3nsc(S(C)(=O)=O)n3)cc2)c1)C(C)C] | 1-7 |
| 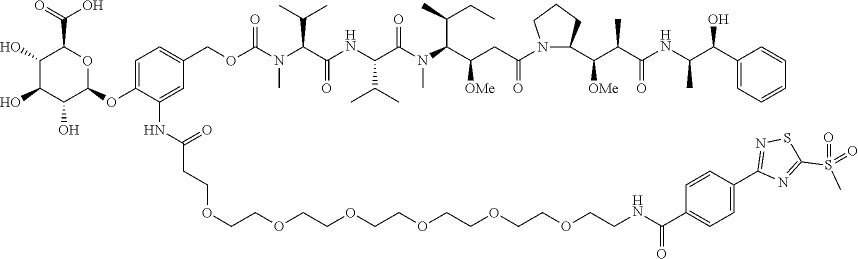 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2O[C@H](C(=O)O)[C@@H](O)[C@H](O)[C@H]2O)c(NC(=O)CCOCCOCCOCCOCCOCCOCCNC(=O)c2ccc(-c3nsc(S(C)(=O)=O)n3)cc2)c1)C(C)C] | 1-8 |
| 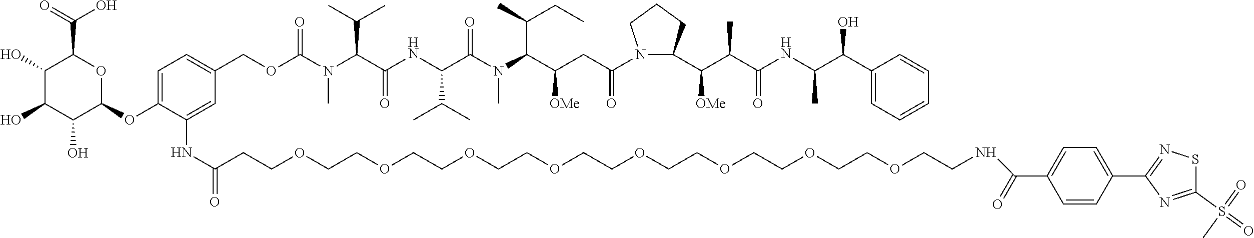 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2O[C@H](C(=O)O)[C@@H](O)[C@H](O)[C@H]2O)c(NC(=O)CCOCCOCCOCCOCCOCCOCCOCCOCCNC(=O)c2ccc(-c3nsc(S(C)(=O)=O)n3)cc2)c1)C(C)C] | 1-9 |
| 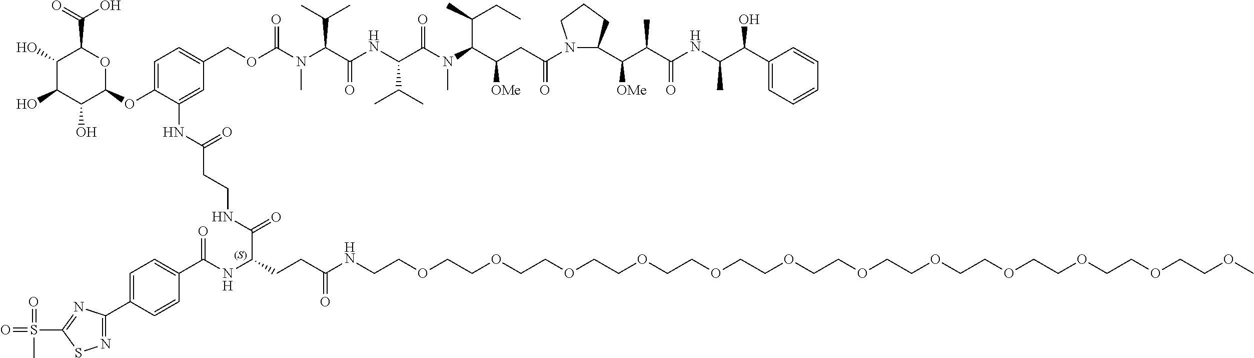 | 1-10 |
| 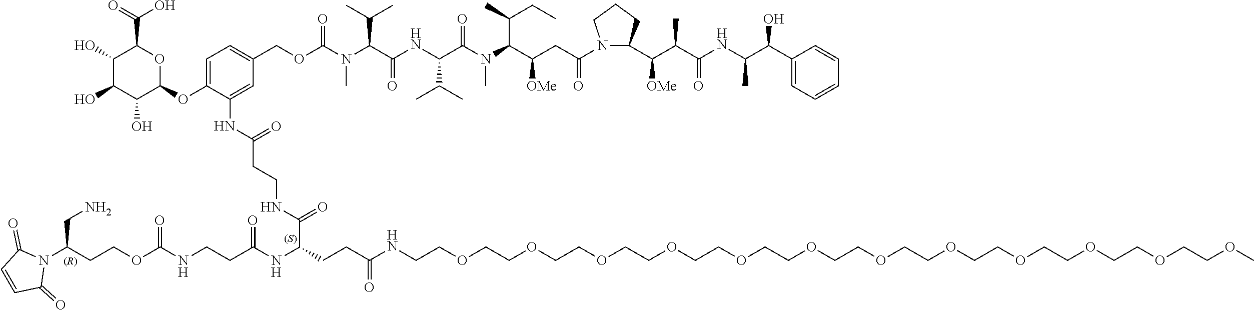 | 1-11 |
| 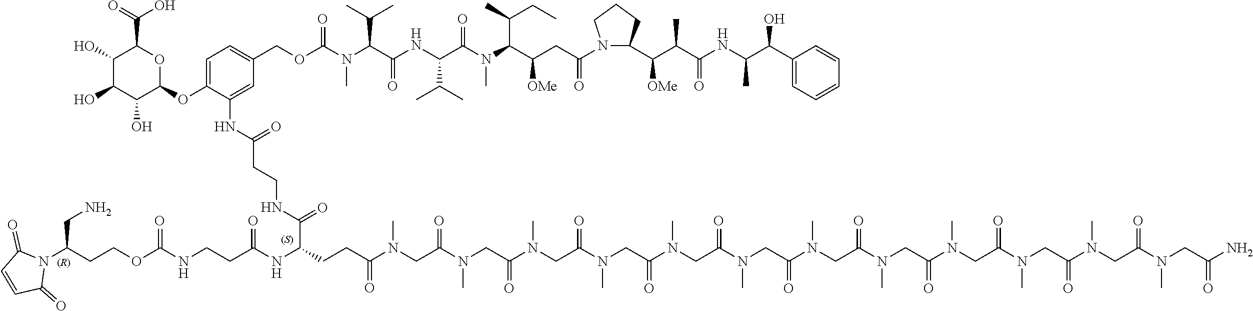 | 1-12 |
| 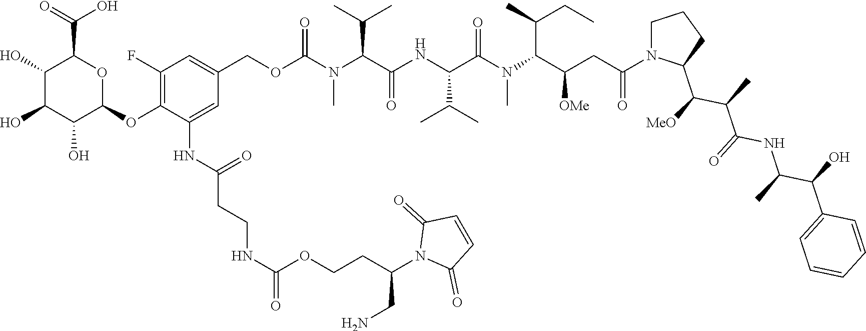 [SMILES: CC[C@H](C)[C@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1cc(F)c(O[C@@H]2O[C@H](C(=O)O)[C@@H](O)[C@H](O)[C@H]2O)c(NC(=O)CCNC(=O)OCC[C@H](CN)N2C(=O)C=CC2=O)c1)C(C)C] | 1-13 |
| 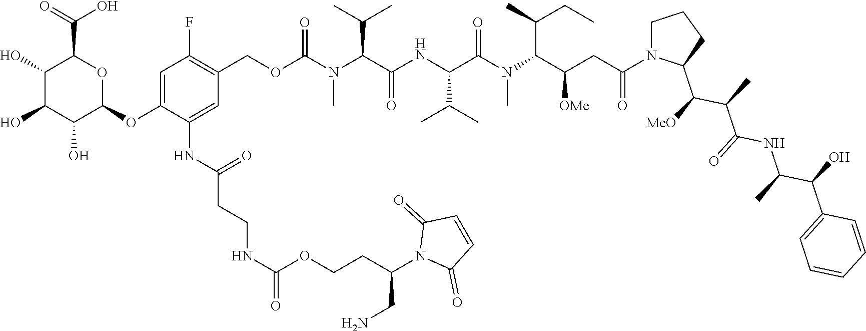 [SMILES: CC[C@H](C)[C@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1cc(NC(=O)CCNC(=O)OCC[C@H](CN)N2C(=O)C=CC2=O)c(O[C@@H]2O[C@H](C(=O)O)[C@@H](O)[C@H](O)[C@H]2O)cc1F)C(C)C] | 1-14 |
| 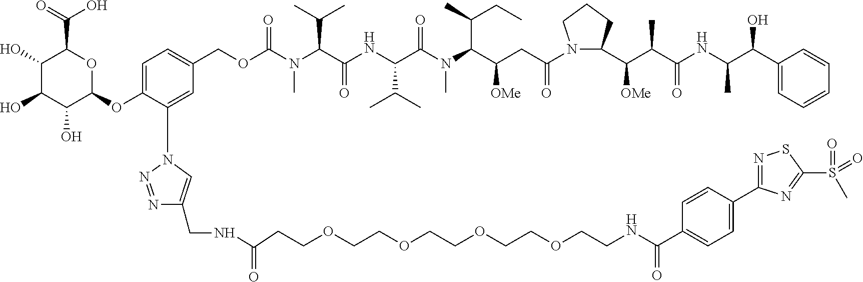 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2O[C@H](C(=O)O)[C@@H](O)[C@H](O)[C@H]2O)c(-n2cc(CNC(=O)CCOCCOCCOCCOCCNC(=O)c3ccc(-c4nsc(S(C)(=O)=O)n4)cc3)nn2)c1)C(C)C] | 1-15 |
| 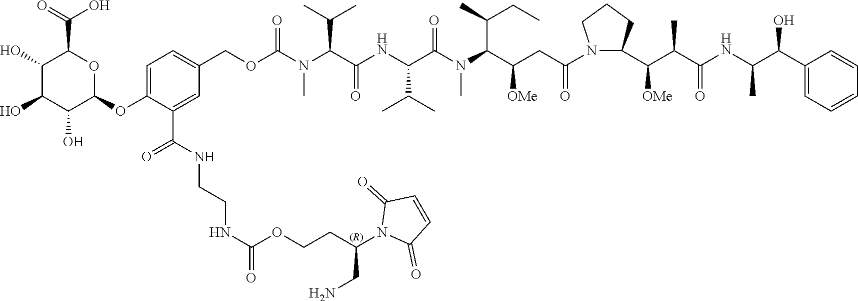 | 1-16 |
| 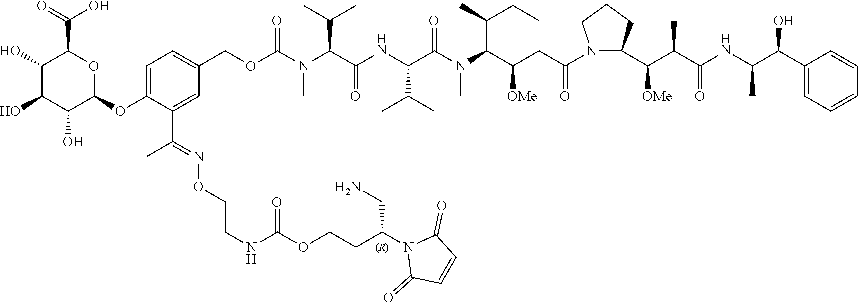 | 1-17 |
| 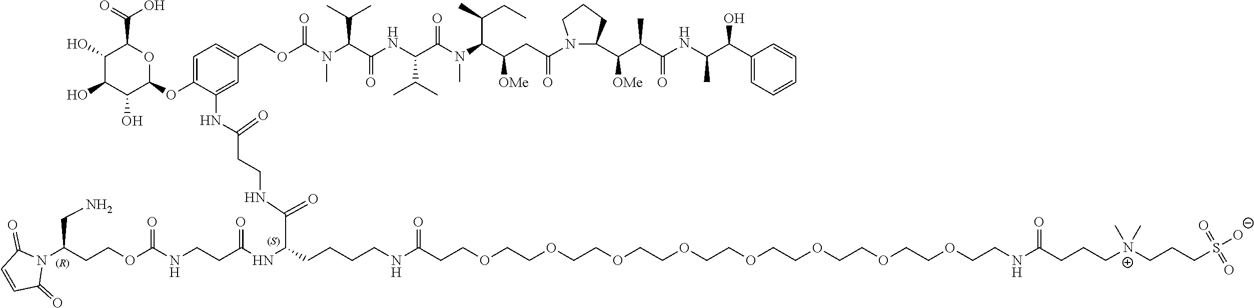 | 1-18 |
| 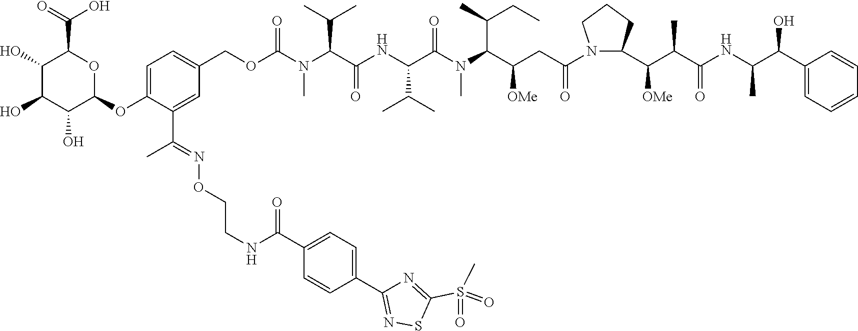 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2O[C@H](C(=O)O)[C@@H](O)[C@H](O)[C@H]2O)c(/C(C)=N/OCCNC(=O)c2ccc(-c3nsc(S(C)(=O)=O)n3)cc2)c1)C(C)C] | 1-19 |
| 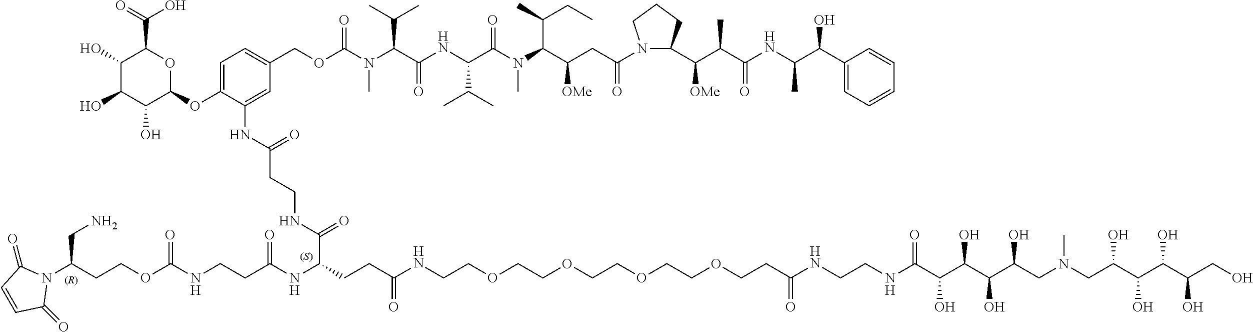 | 1-20 |
| 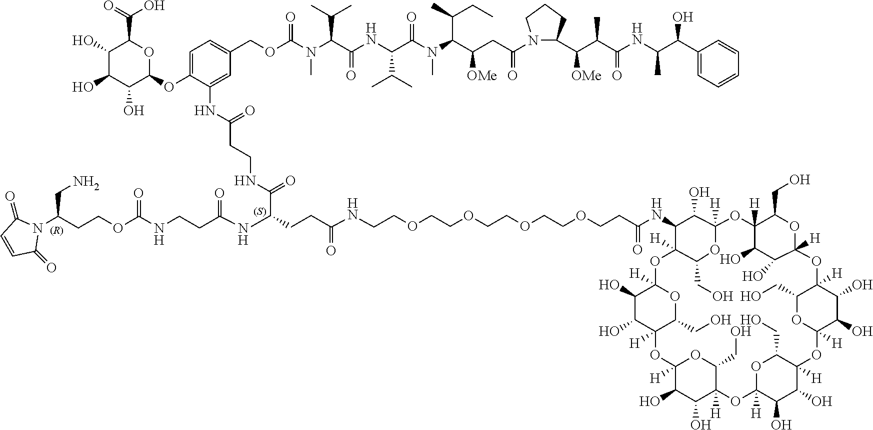 | 1-21 |
| 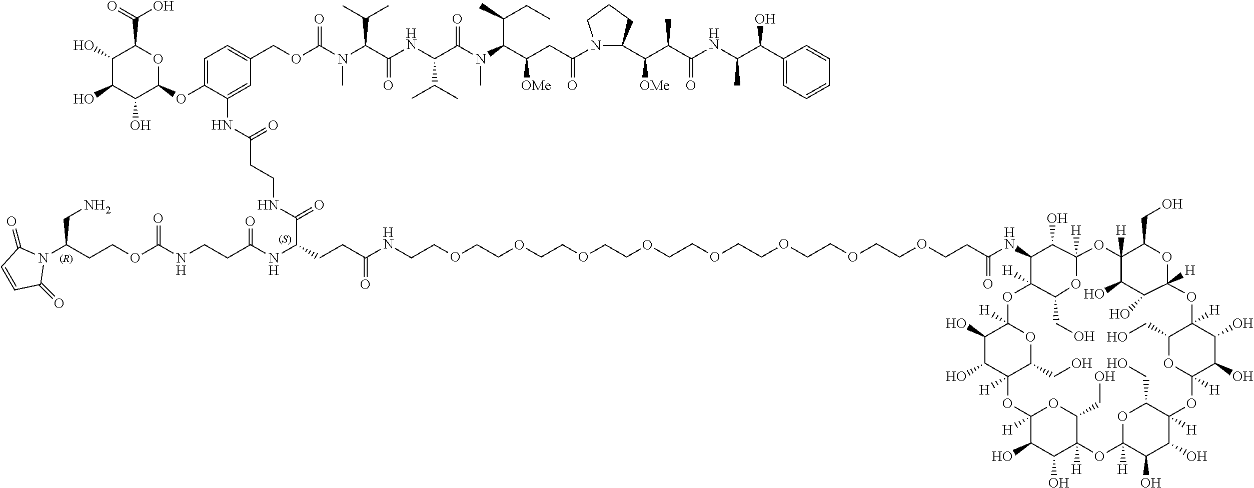 | 1-22 |
| 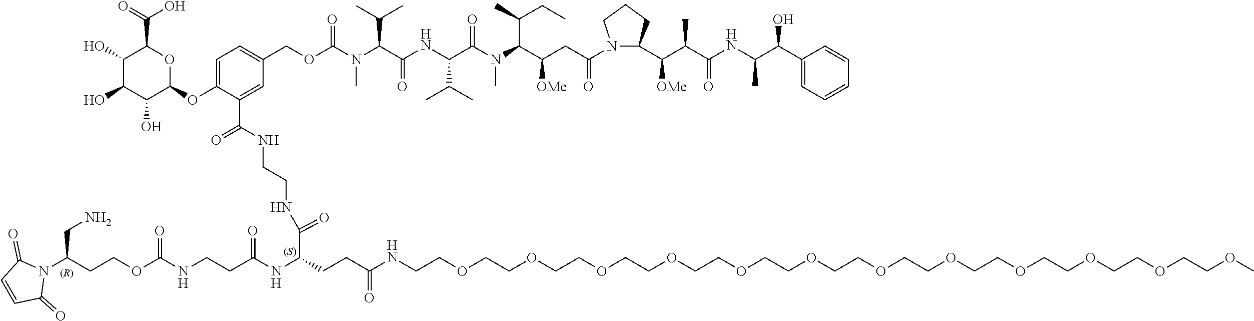 | 1-23 |
| 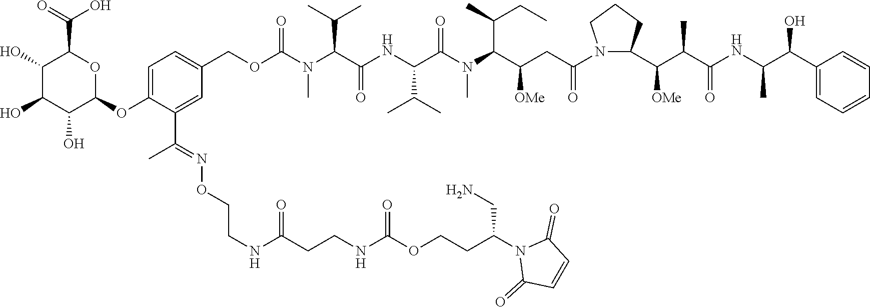 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2O[C@H](C(=O)O)[C@@H](O)[C@H](O)[C@H]2O)c(/C(C)=N/OCCNC(=O)CCNC(=O)OCC[C@H](CN)N2C(=O)C=CC2=O)c1)C(C)C] | 1-24 |
| 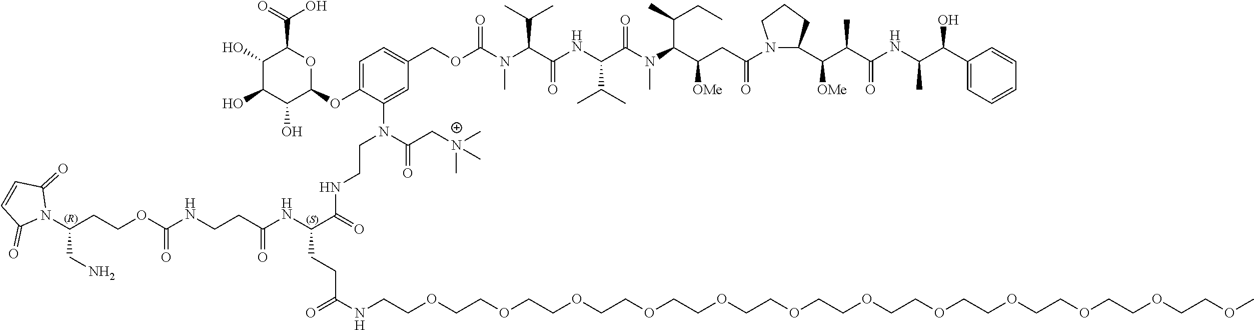 | 1-25 |
| 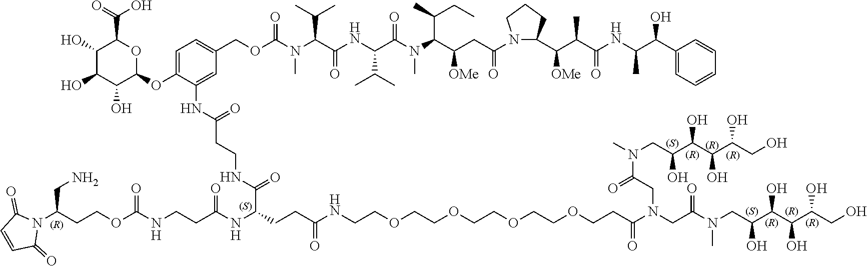 | 1-26 |

TABLE 2A
Isotype ADC Prepared with anti-HIV Isotype Antibody as ″Ab″

| | | DAR8 |
|---|---|---|
| ADC | | RT |
| No. | ADC Structure | (min) |
| Iso- type | 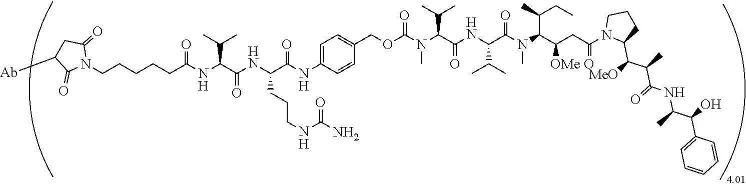 | 14.288 |

TABLE 2B
ADCs Prepared with 6E7 as ″Ab″

| | | DAR8 |
|---|---|---|
| ADC | | RT |
| No. | ADC Structure | (min) |
| 3-A | 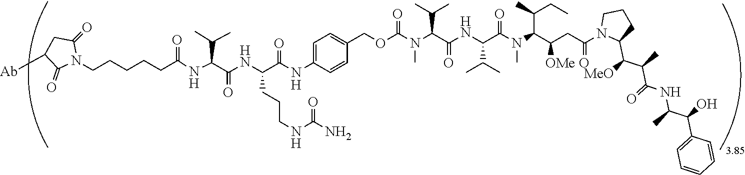 | 14.466 |
| 3-1 | 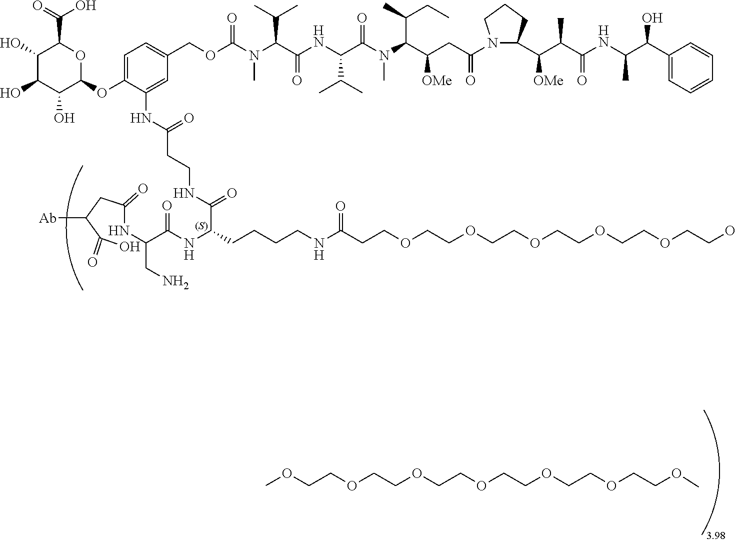 | 13.088 |
| 3-2 | 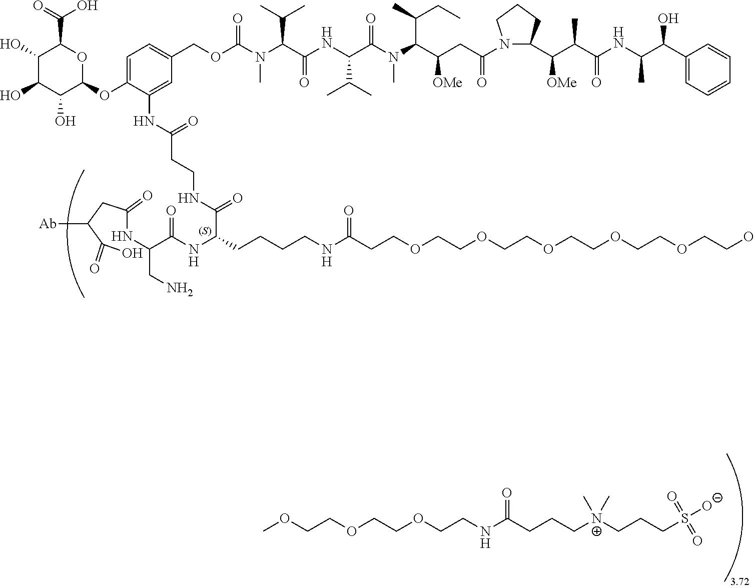 | 12.166 |
| 3-3 | 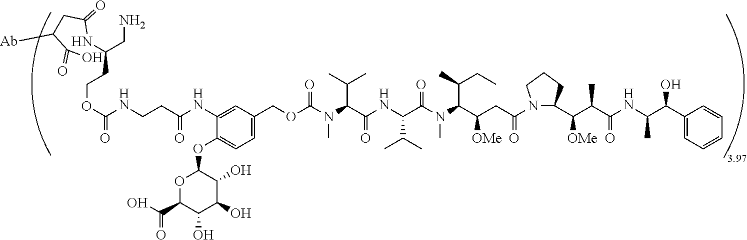 | 12.980 |
| 3-4 | 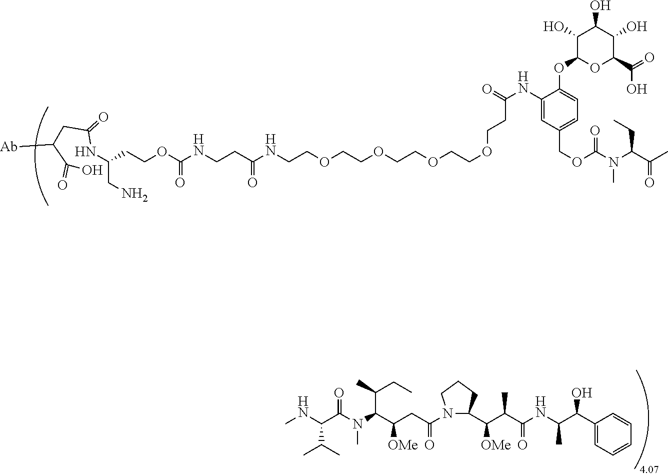 | 13.697 |
| 3-5 | 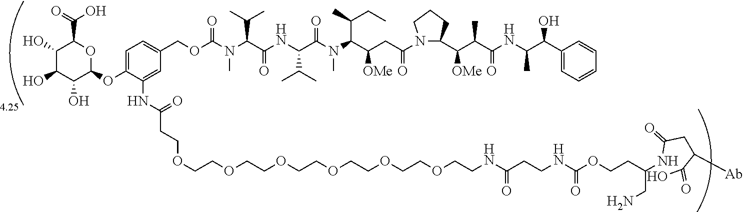 | 13.794 |
| 3-6 | 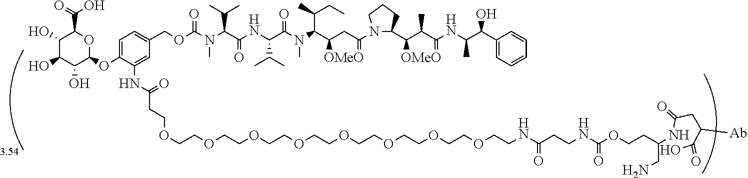 | 13.911 |
| 3-7 | 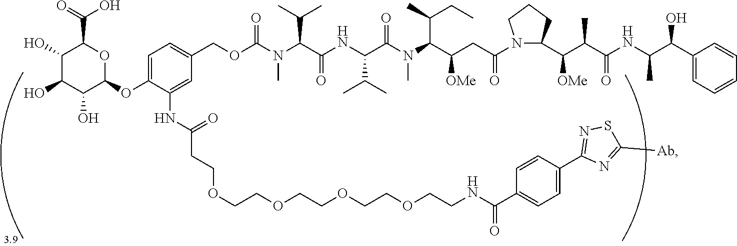 | 12.533 |
| 3-8 | 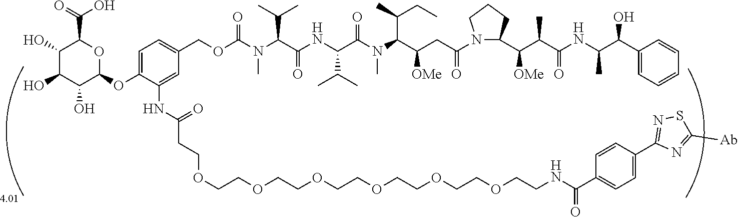 | 12.809 |
| 3-9 | 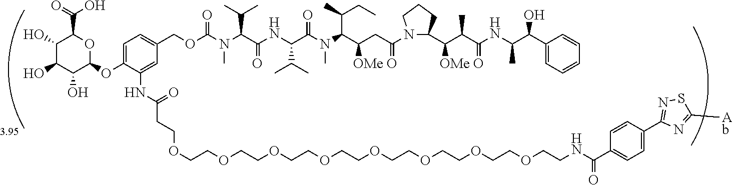 | 12.915 |
| 3-10 | 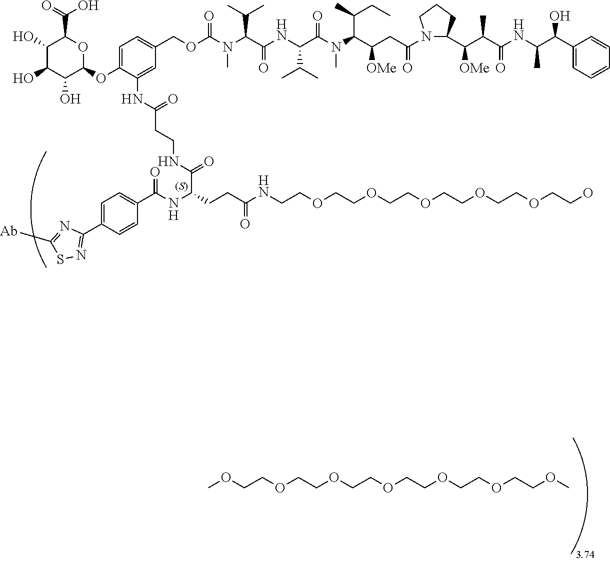 | 12.206 |
| 3-11 | 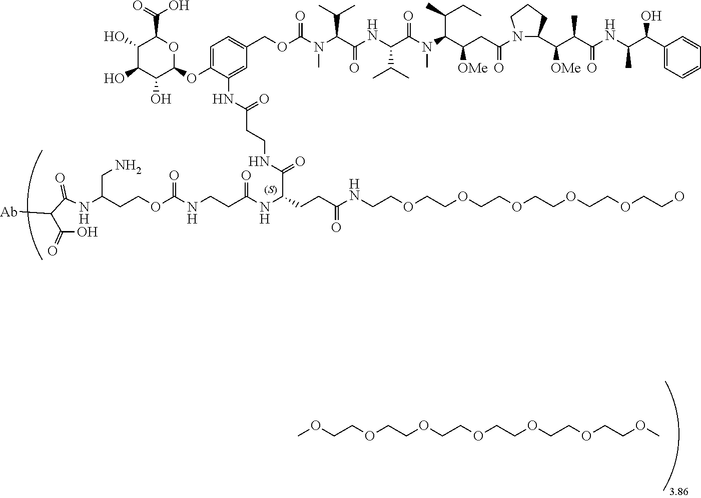 | 13.220 |
| 3-12 | 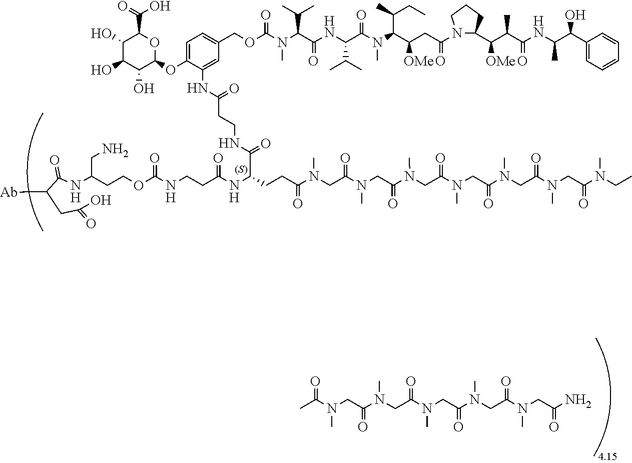 | 11.126 |
| 3-13 | 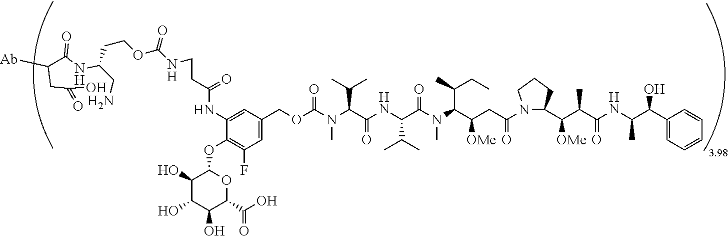 | 13.148 |
| 3-14 | 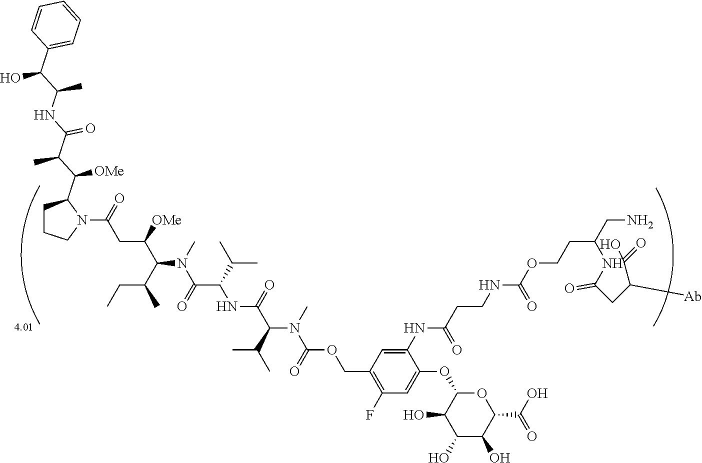 | 12.877 |
| 3-15 | 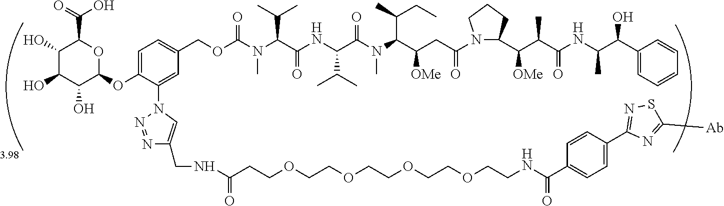 | 12.997 |
| 3-16 | 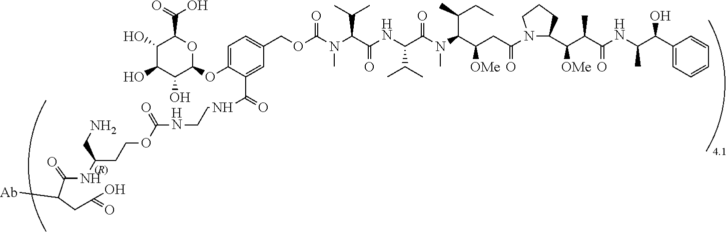 | 12.81 |
| 3-17 | 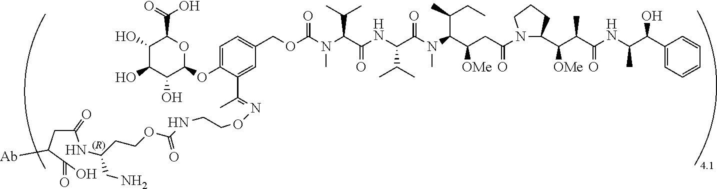 | 13.851 |
| 3-18 | 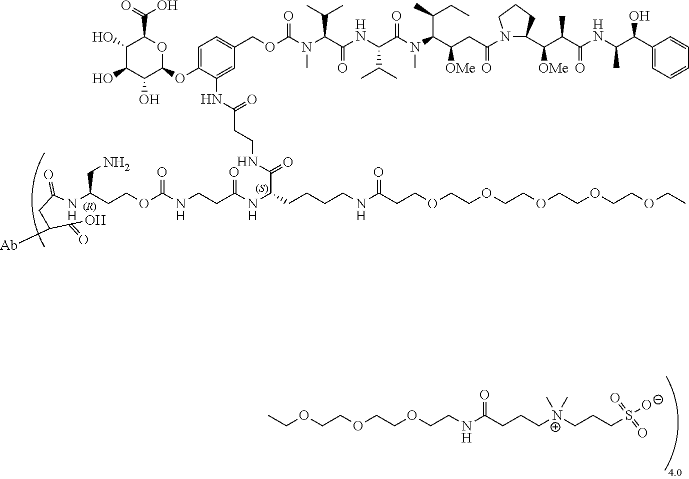 | 12.192 |
| 3-19 | 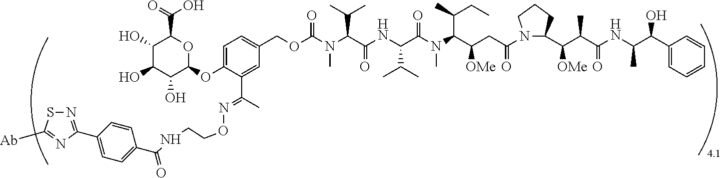 | 13.02 |
| 3-20 | 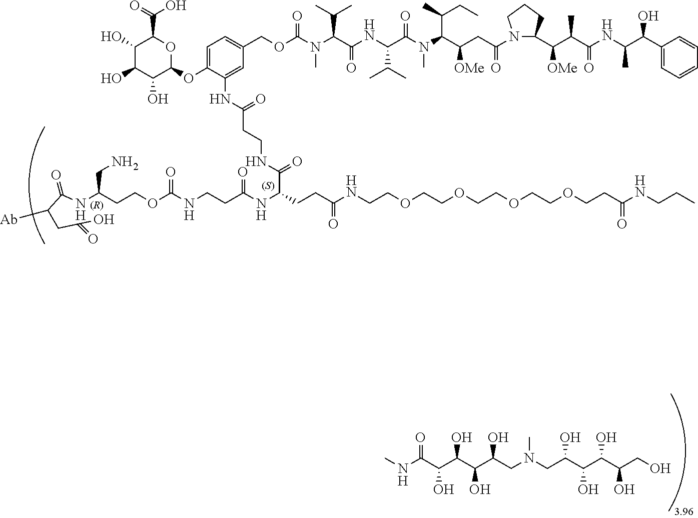 | 12.443 |
| 3-21 | 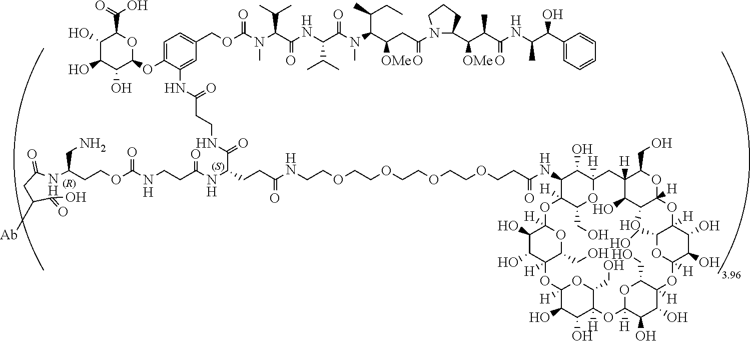 | 12.958 |
| 3-22 | 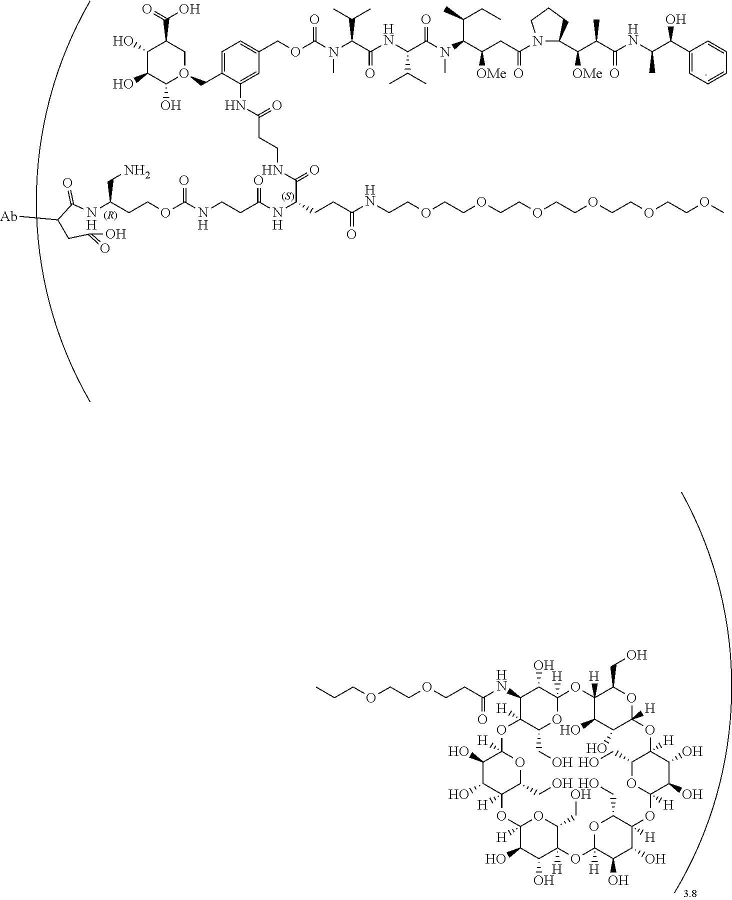 | 12.766 |
| 3-23 | 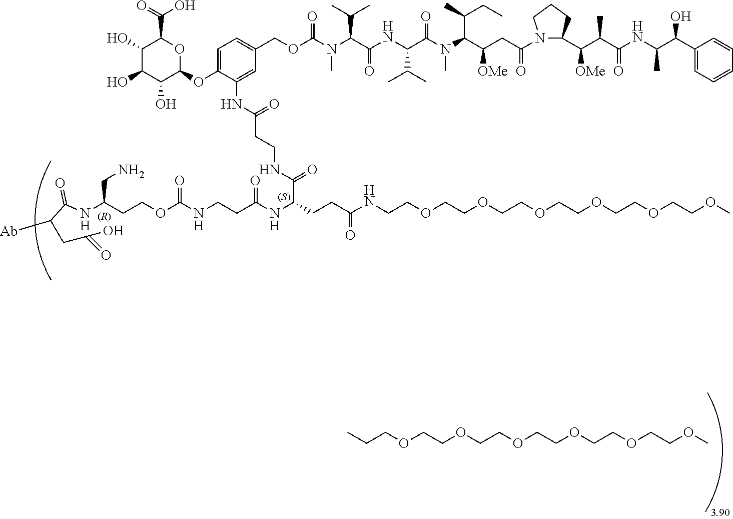 | 12.516 |
| 3-24 | 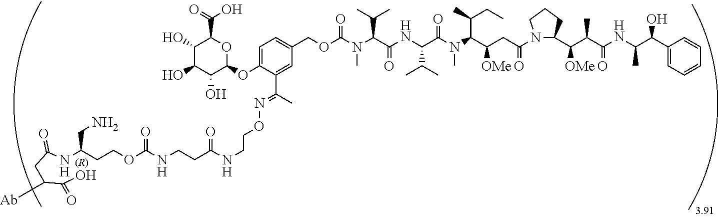 | 14.091 |
| 3-25 | 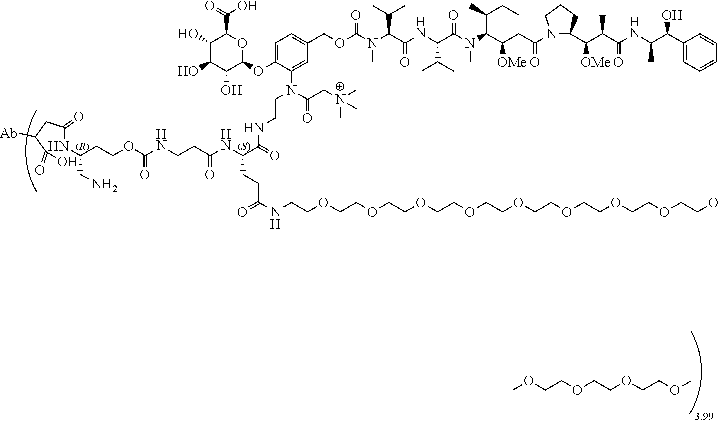 | 11.846 |
| 3-26 | 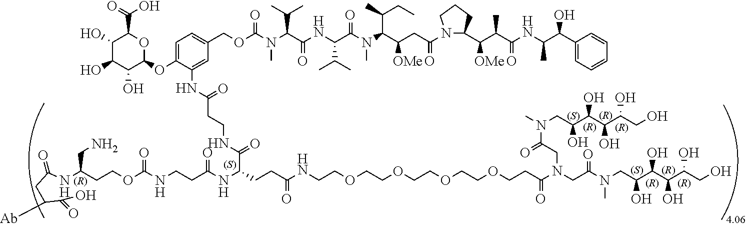 | 11.491 |

TABLE 2C
ADCs Prepared with Infinatamab as ″Ab″

| | | DAR8 |
|---|---|---|
| ADC | | RT |
| No. | ADC Structure | (min) |
| 4-A | 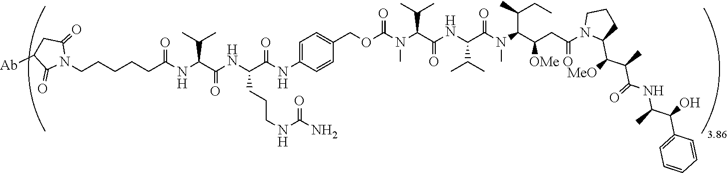 | 14.331 |
| 4-1 | 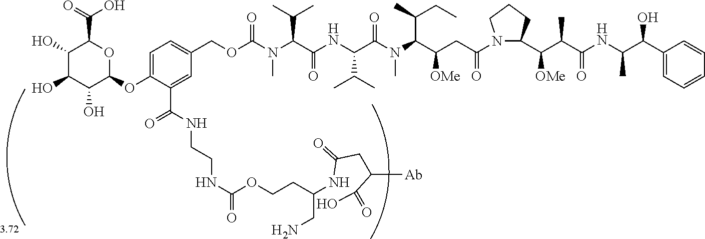 | 12.671 |
| 4-3 | 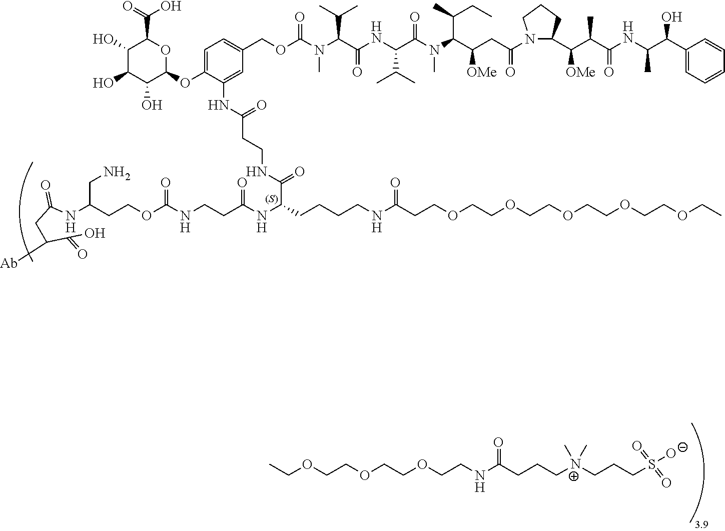 | 12.372 |

TABLE 2D
ADCs Prepared with Cofetuzumab as ″Ab″

| | | DAR8 |
|---|---|---|
| ADC | | RT |
| No. | ADC Structure | (min) |
| 5-A | 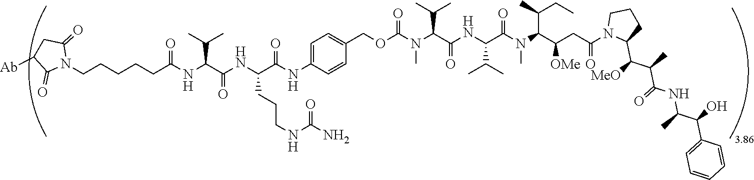 | 13.595 |
| 5-1 | 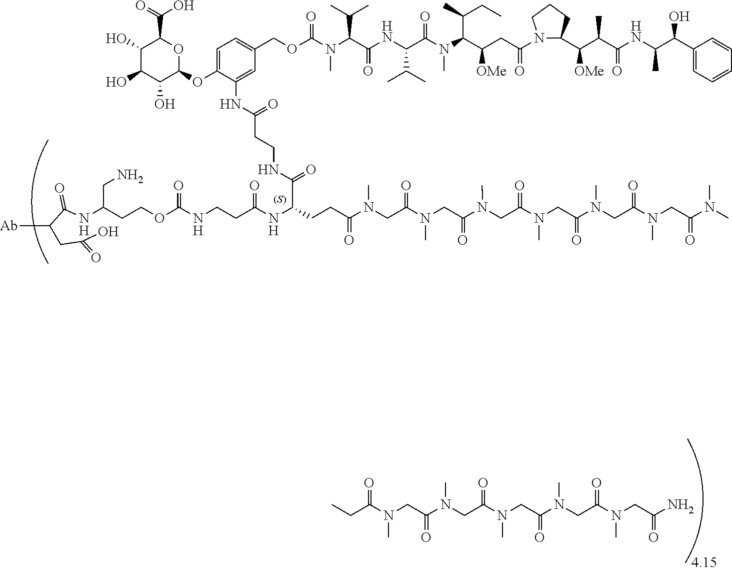 | 11.633 |
| 5-2 | 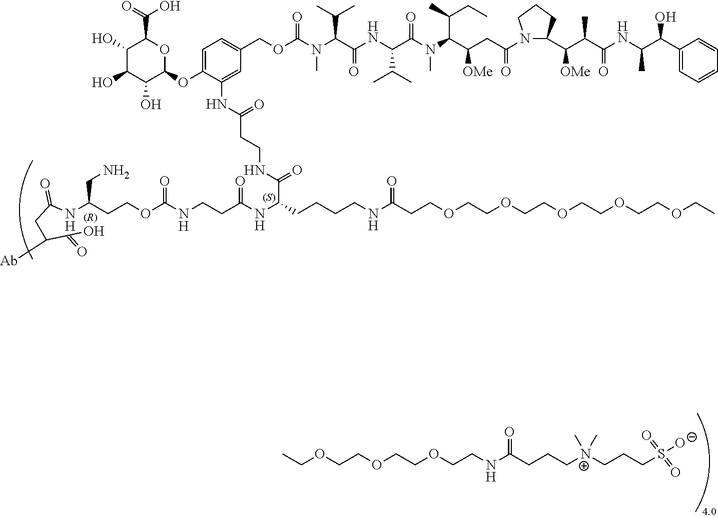 | 12.389 |

### Antibody Information

Ifinatamab (anti-B7H3 antibody)
Light chain sequence
(SEQ ID NO: 1)
EIVLTQSPATLSLSPGERATLSCRASSRLIYMHWYQQKPGQAPRP
LIYATSNLASGIPARFSGSGSGTDFTLTISSLEPEDFAVYYCQQW
NSNPPTFGQGTKVEIKRTVAAPSVFIFPPSDEQLKSGTASVVCLL
NNFYPREAKVQWKVDNALQSGNSQESVTEQDSKDSTYSLSSTLTL
SKADYEKHKVYACEVTHQGLSSPVTKSENRGEC
Heavy chain sequence
(SEQ ID NO: 2)
QVQLVQSGAEVKKPGSSVKVSCKASGYTFTNYVMHWVRQAPGQGL
EWMGYINPYNDDVKYNEKFKGRVTITADESTSTAYMELSSLRSED
TAVYYCARWGYYGSPLYYFDYWGQGTLVTVSSASTKGPSVFPLAP
SSKSTSGGTAALGCLVKDYFPEPVTVSWNSGALTSGVHTFPAVLQ
SSGLYSLSSVVTVPSSSLGTQTYICNVNHKPSNTKVDKRVEPKSC
DKTHTCPPCPAPELLGGPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
EEMTKNQVSLTCLVKGFYPSDIAVEWESNGQPENNYKTTPPVLDS
DGSFFLYSKLTVDKSRWQQGNVFSCSVMHEALHNHYTQKSLSLSP
GK
6E7 (anti-CLL1 antibody)
Light chain sequence
(SEQ ID NO: 3)
DIQMTQSPSSLSASVGDRVTITCRASQSVSTSSYNYMHWYQQKPG
KPPKLLIKYASNLESGVPSRFSGSGSGTDFTLTISSLQPEDFATY
YCQHSWEIPLTFGQGTKVEIKRTVAAPSVFIFPPSDEQLKSGTAS
VVCLLNNFYPREAKVQWKVDNALQSGNSQESVTEQDSKDSTYSLS
STLTLSKADYEKHKVYACEVTHQGLSSPVTKSFNRGEC
Heavy chain sequence
(SEQ ID NO: 4)
EVQLVQSGAEVKKPGASVKVSCKASGYSFTDYYMHWVRQAPGQGL
EWIGRINPYAGAAFYSQNFKDRVTLTVDTSTSTAYLELSSLRSED
TAVYYCAIERGADLEGYAMDYWGQGTLVTVSSASTKGPSVFPLAP
SSKSTSGGTAALGCLVKDYFPEPVTVSWNSGALTSGVHTFPAVLQ
SSGLYSLSSVVTVPSSSLGTQTYICNVNHKPSNTKVDKKVEPKSC
DKTHTCPPCPAPELLGGPSVFLFPPKPKDTLMISRTPEVTCVVVD
VSHEDPEVKENWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GK
Cofetuzumab (anti-PTK7 antibody)
Light chain sequence
(SEQ ID NO: 5)
DIEMTQSPDSLAVSLGERATINCRSSQSVLYSSSNRNYLAWYQQN
PGQPPKLLIYWASTRESGVPDRFSGSGSGTDFTLTISSLQAEDVA
VYYCQQYYSTPRTFGQGTKVEIKRTVAAPSVFIFPPSDEQLKSGT
ASVVCLLNNFYPREAKVQWKVDNALQSGNSQESVTEQDSKDSTYS
LSSTLTLSKADYEKHKVYACEVTHQGLSSPVTKSFNRGEC
Heavy chain sequence
(SEQ ID NO: 6)
QVQLVQSGPEVKKPGASVKVSCKASGYTFTDYAVHWVRQAPGKRL
EWIGVISTYNDYTYNNQDFKGRVTMTRDTSASTAYMELSRLRSED
TAVYYCARGNSYFYALDYWGQGTSVTVSSASTKGPSVFPLAPSSK
STSGGTAALGCLVKDYFPEPVTVSWNSGALTSGVHTFPAVLQSSG
LYSLSSVVTVPSSSLGTQTYICNVNHKPSNTKVDKKVEPKSCDKT
HTCPPCPAPELLGGPSVFLFPPKPKDTLMISRTPEVTCVVVDVSH
EDPEVKFNWYVDGVEVHNAKTKPREEQYNSTYRVVSVLTVLHQDW
LNGKEYKCKVSNKALPAPIEKTISKAKGQPREPQVYTLPPSREEM
TKNQVSLTCLVKGFYPSDIAVEWESNGQPENNYKTTPPVLDSDGS
FFLYSKLTVDKSRWQQGNVFSCSVMHEALHNHYTQKSLSLSPGK

### Example 3: ADC Direct Killing in U937, HL60, TF-1, NCI-H358, NCI-H1048, and MDA-MB-453 cancer lines

### Cell Lines

U937 (ATCC, CRL-1593.2). U-937 is a cell line exhibiting monocyte morphology that was derived in 1974 from malignant cells obtained from the pleural effusion of a 37-year-old white male with histiocytic lymphoma. U937 was purchased from ATCC. The base medium for U937 is ATCC-formulated RPMI-1640 Medium (ATCC 30-2001). To make the complete growth medium, fetal bovine serum to a final concentration of 10% (Gibco, 10099-141C) was added to the base medium. The cell line was grown in a humidified 5% CO₂ atmosphere at 37° C., and was regularly tested for the presence of mycoplasma with MycoAtert™ PLUS Mycoplasma Detection Kit (Lonza, LT07-710).

HL60 (ATCC, CCL-240). HL-60 cells are promyeoloblasts isolated from the peripheral blood by leukopheresis from a 36-year-old white female with acute promyelocytic leukemia. HL60 was purchased from ATCC. The base medium for HL60 is ATCC-formulated Iscove's Modified Dulbecco's Medium, Catalog No. 30-2005. To make the complete growth medium, fetal bovine serum to a final concentration of 20% (Gibco, 10099-141C) was added to the base medium. The cell line was grown in a humidified 5% CO₂ atmosphere at 37° C., and was regularly tested for the presence of mycoplasma with MycoAtert™ PLUS Mycoplasma Detection Kit (Lonza, LT07-710).

TF1 (ATCC, CRL-2003). TF-1 erythroblasts were isolated in 1987 from bone marrow derived from a 35-year-old Asian male with severe pancytopenia. TF-1 was purchased from ATCC. The base medium for TF-1 is ATCC-formulated RPMI-1640 Medium, Catalog No. 30-2001. To make the complete growth medium, fetal bovine serum to a final concentration of 10% (Gibco, 10099-141C) was added to the base medium. The cell line was grown in a humidified 5% CO₂ atmosphere at 37° C., and was regularly tested for the presence of mycoplasma with MycoAtert™ PLUS Mycoplasma Detection Kit (Lonza, LT07-710).

NCI-H358 (ATCC, CRL-5807). NCI-H358 cells are epithelial-like cells isolated from the bronchiole of a male patient with bronchioalveolar carcinoma. NCI-H358 was purchased from ATCC. The base medium for NCI-H358 is ATCC-formulated RPMI-1640 Medium, ATCC 30-2001. To make the complete growth medium, fetal bovine serum to a final concentration of 10% (Gibco, 10099-141C) was added to the base medium. The cell line was grown in a humidified 5% CO₂ atmosphere at 37° C. and was regularly tested for the presence of mycoplasma with MycoAtert™ PLUS Mycoplasma Detection Kit (Lonza, LT07-710).

NCI-H1048 (ATCC, CRL-5853). NCI-H1048 is a cell line exhibiting epithelial morphology. NCI-H1048 was purchased from ATCC. The base medium for NCI-H1048 is ATCC-formulated DMEM:F12 (Catalog No. 30-2006). To make the complete growth medium, fetal bovine serum to a final concentration of 10% (Gibco, 10099-141C) was added to the base medium. The cell line was grown in a humidified 5% CO₂ atmosphere at 37° C., and was regularly tested for the presence of mycoplasma with MycoAtert™ PLUS Mycoplasma Detection Kit (Lonza, LT07-710).

MDA-MB-453 (SIBS). MDA-MB-453 was derived from an effusion of a 48-year-old female patient with metastatic carcinoma of the breast, involving the nodes, brain, and both pleural and pericardial cavities, and MDA-MB-453 was purchased from SIBS. The base medium for MDA-MB-453 is RPMI 1640 Medium, HEPES (Gibco, 22400105). To make the complete growth medium, fetal bovine serum to a final concentration of 10% (Gibco, 10099-141C) was added to the base medium. The cell line was grown in a humidified 5% CO₂ atmosphere at 37° C., and was regularly tested for the presence of mycoplasma with MycoAtert™ PLUS Mycoplasma Detection Kit (Lonza, LT07-710).

TABLE 3
CLL1 expression level

| Cell line | FACS (Emax MFI, 100 nM) | Molecular No. (QSC kit) |
|---|---|---|
| U937 | 1607 | 84858 |
| HL60 | 846 | NA |
| TF-1 | 50 | <5806 |

TABLE 4
B7H3 expression level

| Cell line | Molecular No. (QSC kit) |
|---|---|
| NCI-H358 | 603012 |
| NCI-H1048 | 100687 |
| MDA-MB-453 | <5806 |

ADC direct killing ADC direct killing was assessed in U937, HL60, and TF1 cancer lines. Cells were seeded (U937 or HL60 (3E3/well) or TF1 (6E3/well)) into 2D 96-well plates (Greiner: 655090), 100 μl/well (including 150 μg/ml Fe blocker), and incubated at 37° C., 5% CO₂, for 1 h. Fresh growth medium was added containing varying concentrations of ADCs, 50 μl/well, and incubated at 37° C., 5% CO₂, for 6 days. The cell viability was detected by Cell Titer-Glo (Promega, G7573), 70 μl/well. The 2D plates were allowed to incubate at room temperature for 10 minutes to stabilize the luminescent signal. The plates were analyzed with a microplate reader.

ADC direct killing was assessed in NCI-H358, NCI-H1048, and MDA-MB-453 cancer lines. Cells were seeded (2E3/well) into 3D 96-well plates (Corning: 4520), 80 μl/well, and incubated at 37° C., 5% CO₂, overnight. Fresh growth medium was added containing varying concentrations of ADCs, 40 μl/well, and incubated at 37° C., 5% CO₂, for 6 days. The cell viability was detected by 3D reagent (Promega, G9683), 100 μl/well. The 3D plates were allowed to incubate at room temperature for 30 minutes to stabilize the luminescent signal. The plates were analyzed with a microplate reader.

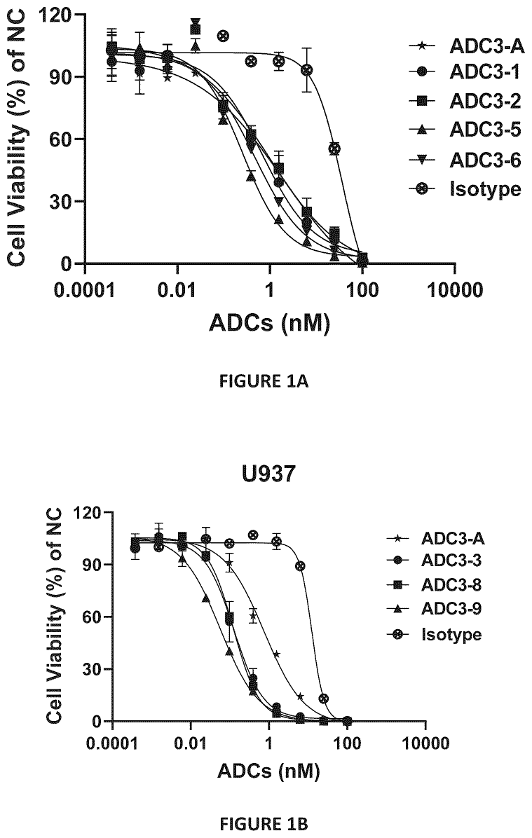

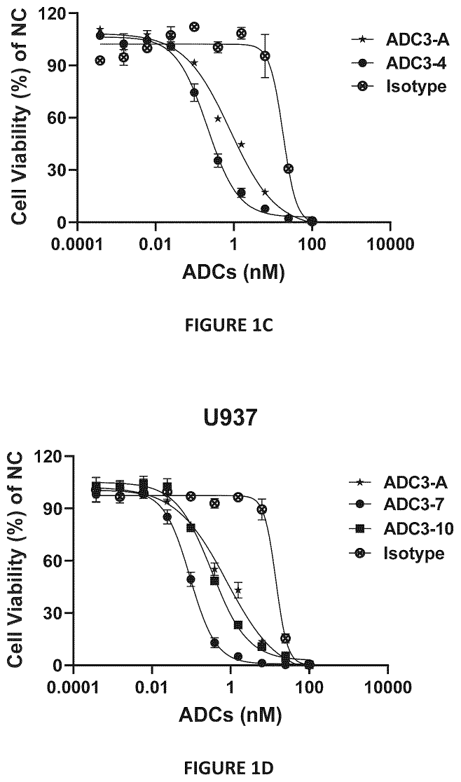

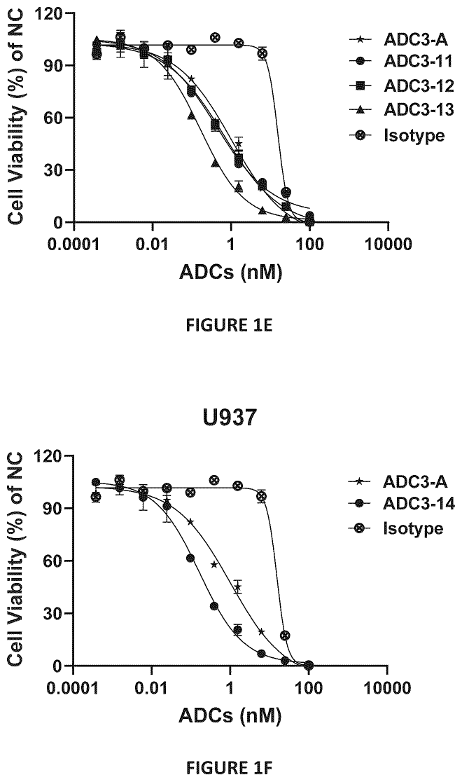

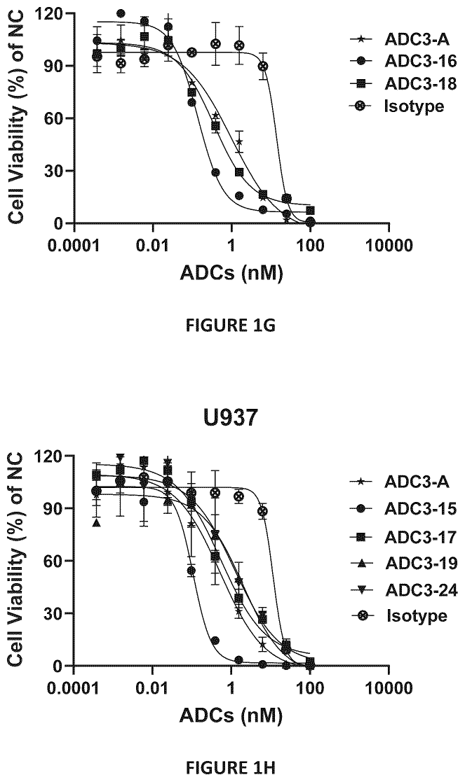

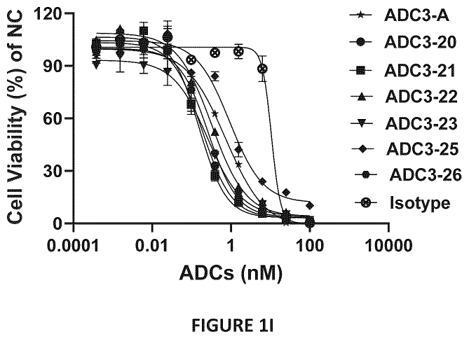

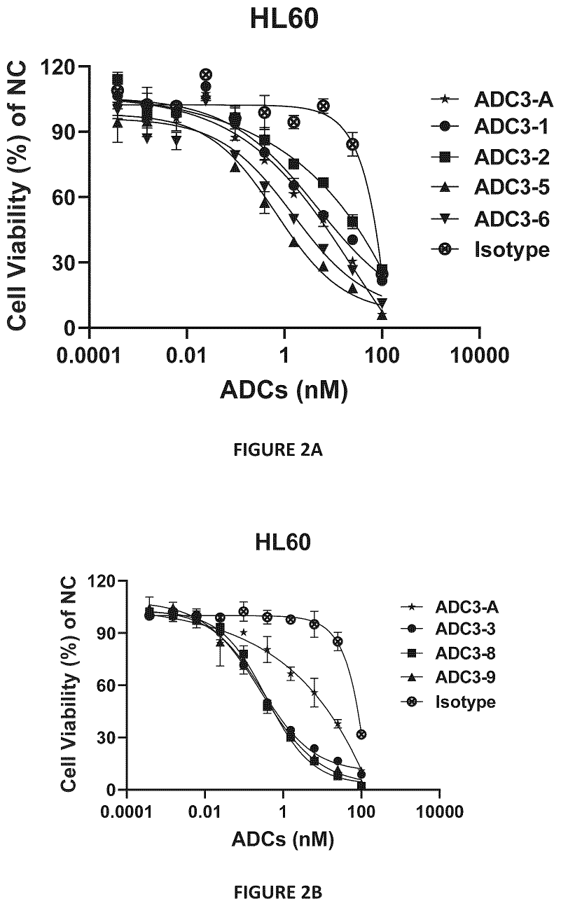

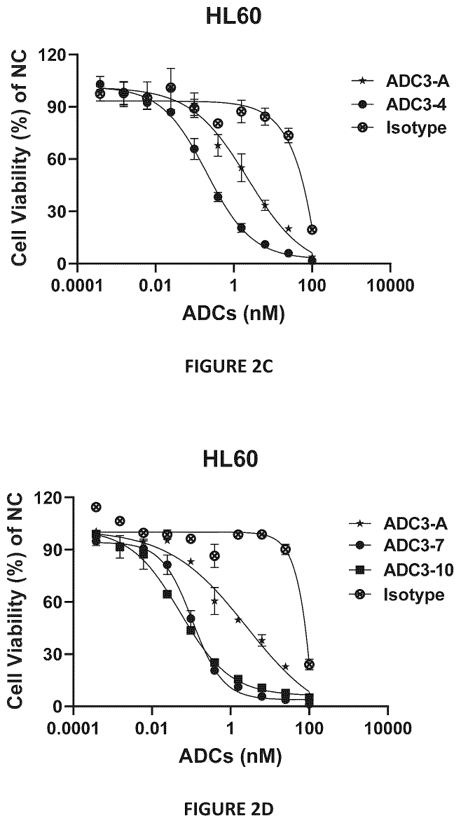

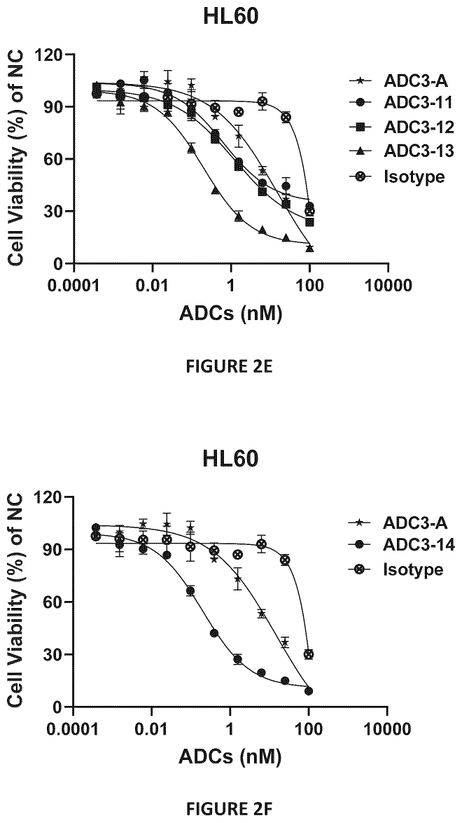

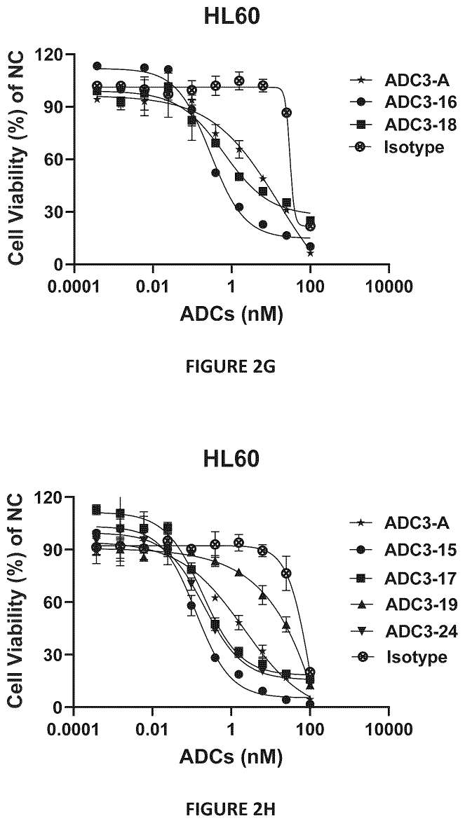

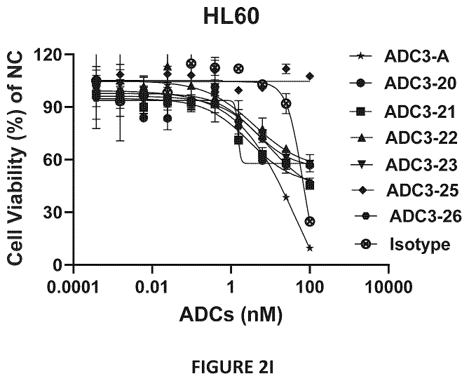

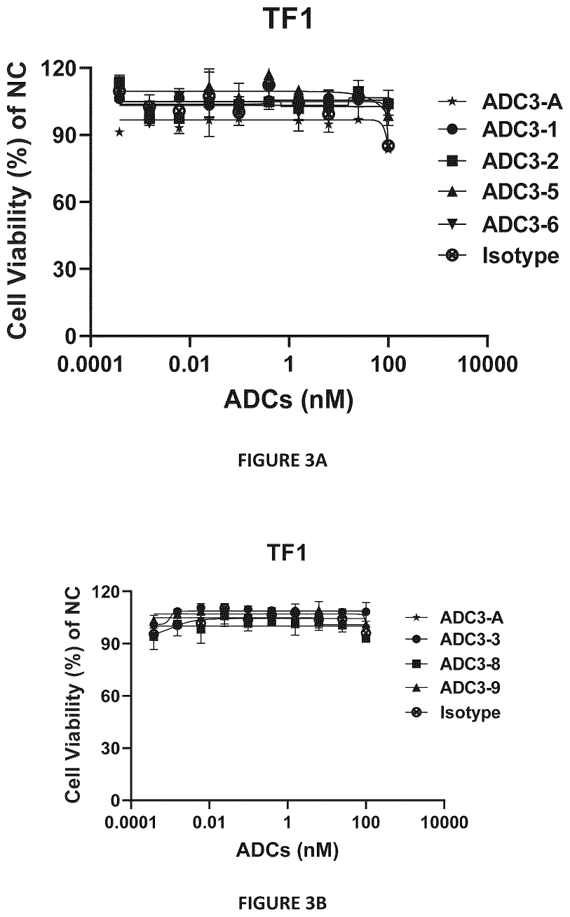

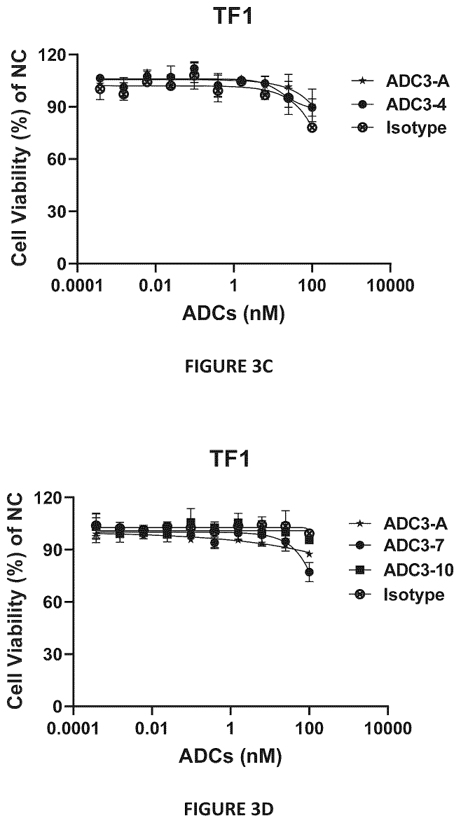

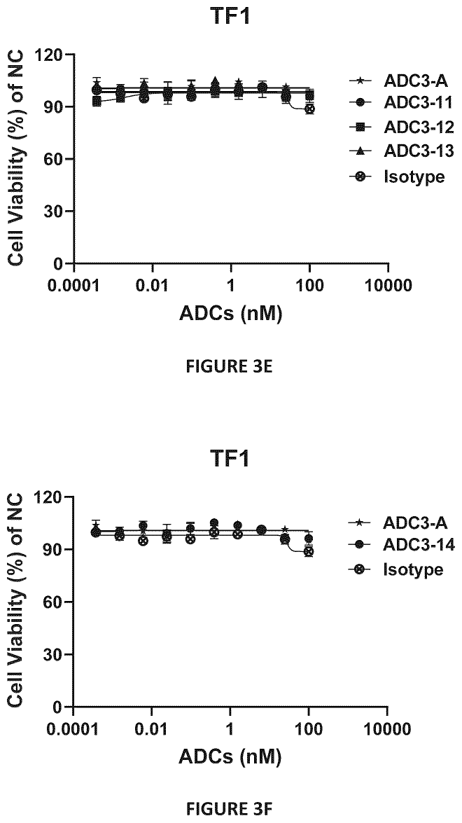

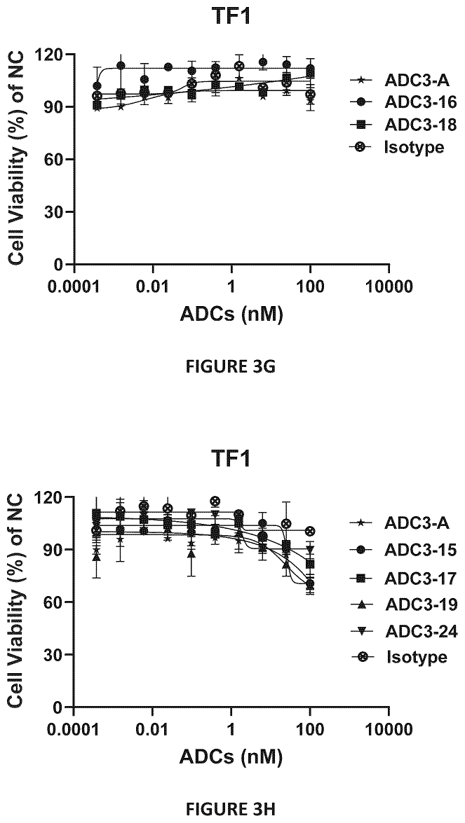

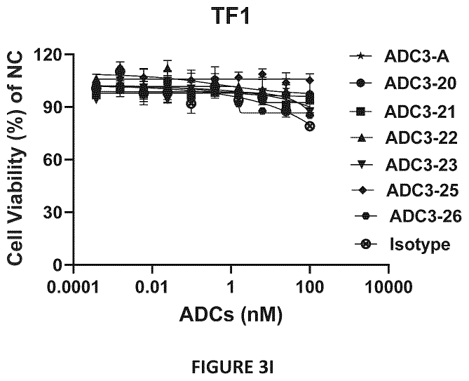

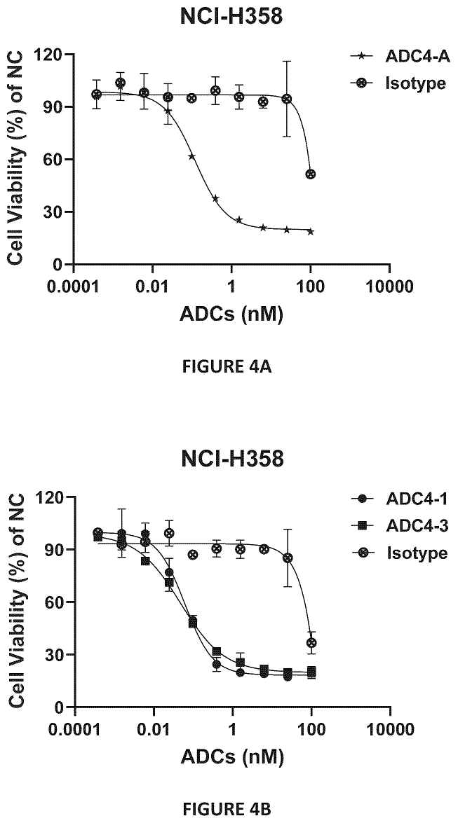

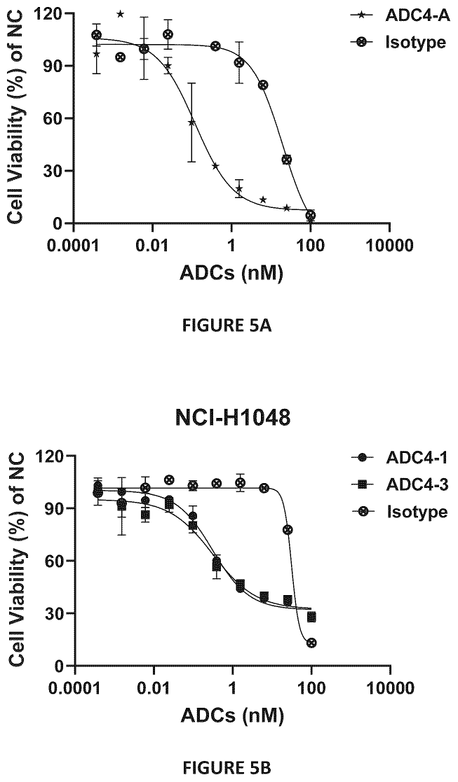

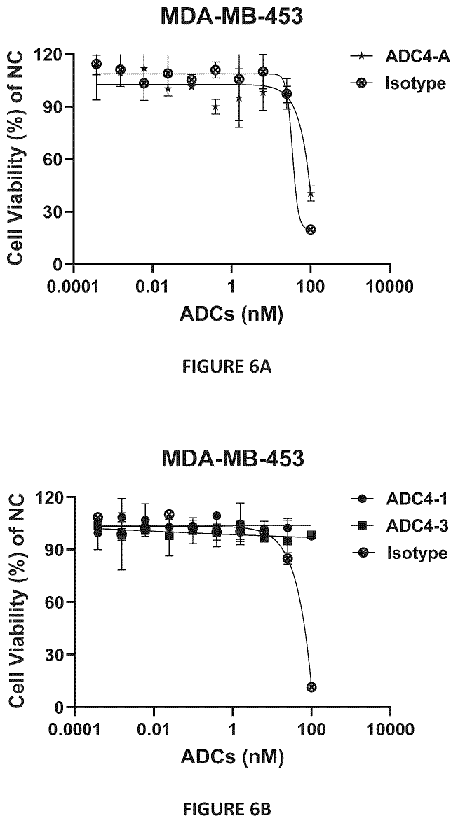

ADC direct cellular killing data are presented in FIGS. 1A-3I and Tables 5-13 for U937, HL60, and TF1 cells. ADC direct cellular killing data are presented in FIGS. 4A-6B and Tables 14 and 15 for NCI-H358, NCI-H1048, and MDA-MB-453 cells.

TABLE 5
ADC direct cellular killing

| | U937 | | HL60 | |
|---|---|---|---|---|
| ADCs | Emax (%) | EC50 (nM) | Emax (%) | EC50 (nM) |
| ADC3-A | 99.8 | 1.0 | 93.9 | 4.3 |
| ADC3-1 | 97.7 | 0.8 | 78.3 | 3.0 |
| ADC3-2 | 97.2 | 1.0 | 73.2 | 7.7 |
| ADC3-5 | 99.4 | 0.3 | 93.9 | 0.6 |
| ADC3-6 | 98.6 | 0.5 | 88.8 | 1.1 |

TABLE 6
ADC direct cellular killing

| | U937 | | HL60 | |
|---|---|---|---|---|
| ADCs | Emax (%) | EC50 (nM) | Emax (%) | EC50 (nM) |
| ADC3-A | 99.9 | 0.8 | 89.0 | 6.3 |
| ADC3-3 | 99.9 | 0.1 | 91.3 | 0.3 |
| ADC3-8 | 99.9 | 0.1 | 97.9 | 0.4 |
| ADC3-9 | 100.0 | 0.1 | 96.4 | 0.4 |

TABLE 7
ADC direct cellular killing

| | U937 | | HL60 | |
|---|---|---|---|---|
| ADCs | Emax (%) | EC50 (nM) | Emax (%) | EC50 (nM) |
| ADC3-A | 99.8 | 0.9 | 96.2 | 1.9 |
| ADC3-4 | 99.7 | 0.2 | 98.3 | 0.2 |

TABLE 8
ADC direct cellular killing

| | U937 | | HL60 | |
|---|---|---|---|---|
| ADCs | Emax (%) | EC50 (nM) | Emax (%) | EC50 (nM) |
| ADC3-A | 99.9 | 0.7 | 94.0 | 1.4 |
| ADC3-7 | 99.9 | 0.1 | 98.8 | 0.1 |
| ADC3-10 | 99.7 | 0.4 | 95.0 | 0.1 |

TABLE 9
ADC direct cellular killing

| | U937 | | HL60 | |
|---|---|---|---|---|
| ADCs | Emax (%) | EC50 (nM) | Emax (%) | EC50 (nM) |
| ADC3-A | 99.9 | 0.9 | 90.7 | 6.5 |
| ADC3-11 | 96.0 | 0.5 | 67.0 | 0.8 |
| ADC3-12 | 99.4 | 0.6 | 76.3 | 1.0 |
| ADC3-13 | 99.7 | 0.2 | 91.0 | 0.2 |

TABLE 10
ADC direct cellular killing

| | U937 | | HL60 | |
|---|---|---|---|---|
| ADCs | Emax (%) | EC50 (nM) | Emax (%) | EC50 (nM) |
| ADC3-A | 99.9 | 0.9 | 90.7 | 6.5 |
| ADC3-14 | 99.7 | 0.2 | 91.0 | 0.2 |

TABLE 11
ADC direct cellular killing

| | U937 | | HL60 | |
|---|---|---|---|---|
| ADCs | Emax (%) | EC50 (nM) | Emax (%) | EC50 (nM) |
| ADC3-A | 99.8 | 0.9 | 93.6 | 4.8 |
| ADC3-16 | 98.6 | 0.2 | 89.7 | 0.4 |
| ADC3-18 | 92.6 | 0.4 | 75.0 | 0.7 |

TABLE 12
ADC direct cellular killing

| | U937 | | HL60 | |
|---|---|---|---|---|
| ADCs | Emax (%) | EC50 (nM) | Emax (%) | EC50 (nM) |
| ADC3-A | 99.9 | 0.6 | 95.5 | 1.2 |
| ADC3-15 | 99.9 | 0.1 | 98.3 | 0.1 |
| ADC3-17 | 97.6 | 0.9 | 83.9 | 0.3 |
| ADC3-19 | 99.9 | 1.6 | 87.4 | 13.4 |
| ADC3-24 | 98.5 | 1.4 | 87.0 | 0.2 |

TABLE 13
ADC direct cellular killing

| | U937 | | HL60 | |
|---|---|---|---|---|
| ADCs | Emax (%) | EC50 (nM) | Emax (%) | EC50 (nM) |
| ADC3-A | 99.9 | 0.7 | 90.5 | 10.7 |
| ADC3-20 | 99.3 | 0.2 | 43.2 | 1.5 |
| ADC3-21 | 99.3 | 0.2 | 54.7 | 2.3 |
| ADC3-22 | 98.9 | 0.4 | 42.1 | 4.2 |
| ADC3-23 | 98.2 | 0.2 | 42.6 | 3.0 |
| ADC3-25 | 89.9 | 1.1 | −12.9 | No killing |
| ADC3-26 | 98.8 | 0.3 | 53.3 | 5.3 |

TABLE 14
ADC direct cellular killing

| | NCI-H358 | | NCI-H1048 | |
|---|---|---|---|---|
| ADCs | Emax (%) | EC50 (nM) | Emax (%) | EC50 (nM) |
| ADC4-A | 81.3 | 0.1 | 98.1 | 0.2 |

TABLE 15
ADC direct cellular killing

| | NCI-H358 | | NCI-H1048 | |
|---|---|---|---|---|
| ADCs | Emax (%) | EC50 (nM) | Emax (%) | EC50 (nM) |
| ADC4-1 | 80.3 | 0.06 | 72.3 | 0.36 |
| ADC4-3 | 79.9 | 0.05 | 72.1 | 0.31 |

### Example 4: ADC PK Studies in Mice

Female BALB/c nude mice (n=3 per group) were treated with freshly prepared ADCs (5 mg/kg) through intravenous administration. At the designated time points after dosing, mice were anesthetized by isoflurane. Whole blood samples were collected from orbital venous sinuses into coagulation tubes (Kangjian, #041-0121) at 0.5 h, 2 h, 6 h, 8 h, 24 h, 48 h, 72 h, and 168 h post-dosing. Blood samples were placed at room temperature for about 30 minutes and processed by centrifugation (4° C., 3000×g, 7 min) to separate plasma. Serum samples were transferred into new 1.5 mL tubes and kept in a −80° C. freezer prior to analysis.

Serum payload concentrations. To determine serum payload concentrations, an aliquot of 15 μL sample was added with 150 μL IS (Verapamil, 10 ng/mL) in ACN/MeOH (1:1). The mixture was vortexed for 1 min and centrifuged at 4000 rpm for 10 min at 4° C. An aliquot of 80 μL supernatant was diluted with 80 μL water, and the mixed sample was injected into LC-MS/MS.

LC-MS MS method to determine payload bioanalysis. LC-MS/MS analysis was carried out under the following measurement conditions:
Instrument: LC-MS/MS (Triple Quad 6500 plus)
Monitor: MRM
Column: Advanced Materials Technology, HALO AQ-C18 2.7 μm 90 Å, 50*2.1 mm
Column temperature: 40° C.
Mobile phase A: H₂O-0.1% FA
Mobile phase B: ACN-0.1% FA
Gradient program for MMAE: 15% B-15% B (0 min-0.4 min), 15% B-30% B (0.4 min-0.8 min), 30% B-30% B (0.8 min-1.8 min), 30% B-90% B (1.8 min-1.9 min), 90% B-90% B (1.9 min-2.4 min), 90% B-15% B (2.4 min-2.5 min), 15% B-15% B (2.5 min-3.0 min)
Injected sample amount: 10 μL (MMAE)

Plasma ADC and total Ab (Tab) concentrations. Plasma ADC and Tab concentrations were determined using an ELISA assay in which the capture reagent was CLL1 or PTK7 (as applicable) ECD (extracellular domain). The detection reagent was anti-payload Ab for ADC concentration and anti-human IgG polyclonal Ab for Tab concentration.

For ADC concentration, microplates were coated with CLL1 or PTK7, as applicable. ECD at 1 μg/mL, 100 μL per well and incubated overnight at 4° C. The microplates were washed 3 times with 0.05% PBST, then 200 μL 2% BSA solution was added per well. The plates were incubated at 37° C. for 1 h followed by washing 3 times with 0.05% PBST. Then 100 μL (of ADC samples or for standard curves) were added to each well. Plates were incubated at 37° C. for 1h followed by washing 3 times with 0.05% PBST. 100 μL of 1 μg/ml biotin anti-MMAE antibody was added per well. Plates were again incubated at 37° C. for 1 h and then washed 3 times with 0.05% PBST. Then 100 μL of 1:10000 SA-HRP was added per well, incubated at 37° C. for 0.5h, followed by washing 3 times with 0.05% PBST. Lastly, 100 μL TMB was added per well and plates were incubated for 15 mins at room temperature before the addition of 100 μL stop solution per well. Plates were read at OD 450 on an ELISA microplate reader.

For Tab concentration of anti-CLL1 antibodies, the foregoing ADC method was used except for the following changes: 100 μL of anti human lgG Fc HRP (1:10000) was added per well.

For Tab concentration of anti-PTK1 antibodies, the foregoing ADC method was used except for the following changes: 100 μL of 1 μg/ml anti human lgG Fc HRP (1:10000) was added per well.

ADC DAR changes in mouse serum PK samples. ADC stability as reflected in DAR decreases over time was studied using the plasma samples described above. The post-dosing timepoints of the samples tested are indicated in Tables 16 and 17 below.

Human CLL1 or PTK7, as applicable, ECD was biotinylated and immobilized onto Dynabeads M-280 Streptavidin. ADCs were captured from the plasma samples by incubation with an ECD-bead system for 2 hours at 37° C. The captured ADCs were then washed with HBS-EP buffer (10 mM Hepes [pH 7.4], 150 mM NaCl, 3.4 mM ethylenediaminetetraacetic acid [EDTA], 0.005% Surfactant P20) and digested using IdeS enzyme at 37° C. for 1 h. After extensive washing of the beads with HBS-EP, water, and 10% acetonitrile, the ADC analytes were eluted using 30% acetonitrile with 1% formic acid. The samples were then reduced with 100 mM TCEP for 45 min. Liquid chromatography-tandem mass spectrometry (LC-MS/MS)methods were used for the ADC DAR analysis.

Mouse PK parameters of some anti-CLL1 ADCs (ADC3-A, ADC3-11, ADC3-12, ADC3-14, and ADC3-18) are presented in Table 16 below. The results demonstrate that ADC3-11, ADC3-12, ADC3-14, and ADC3-18 exhibited lower payload exposure and lower DAR changes than did ADC3-A.

TABLE 16
Mouse PK parameters of anti-CLL1 ADCs

| | | Tab | ADC | Payload | |
|---|---|---|---|---|---|
| | Dose | AUCₗₐₛₜ | AUCₗₐₛₜ | AUCₗₐₛₜ | |
| ADC No. | (mg/kg) | (h · μg/mL) | (h · μg/mL) | (h · ng/mL) | DAR |
| ADC3-A | 5 | 6448 | 4353 | 8.58 | 2.76 (0.5 h) |
| | | | | | 1.96 (72 h) |
| | | | | | 1.22 (168 h) |
| ADC3-11 | 5 | 8559 | 8127 | NA | 3.24 (2 h) |
| | | | | | 2.50 (168 h) |
| ADC3-12 | 5 | 7949 | 8358 | NA | 2.68 (2 h) |
| | | | | | 2.42 (168 h) |
| ADC3-14 | 5 | 6481 | 5072 | NA | 4.04 (2 h) |
| | | | | | 2.88 (168 h) |
| ADC3-18 | 5 | 8242 | 7696 | NA | 3.96 (2 h) |
| | | | | | 2.44 (48 h) |

NA: not available (because limited concentration for AUC calculation as payload concentration below quantitation limit (lowest limit of quantitation = 50 pg/mL))

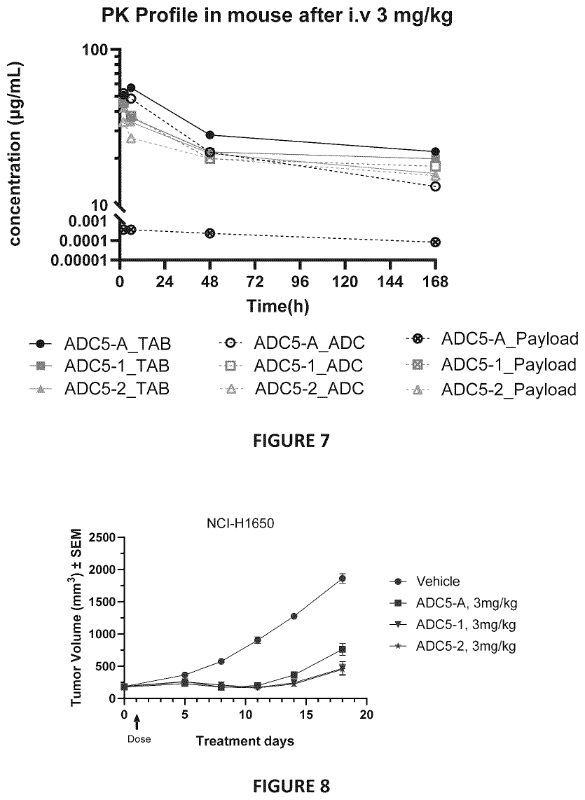

Mouse PK parameters of some anti-PTK7 ADCs (ADC5-A, ADC5-1, and ADC5-2) are presented in Table 17 and FIG. 7. The results demonstrate that ADC5-1 and ADC5-2 exhibited lower payload exposure and lower DAR changes than did ADC5-A.

TABLE 17
Mouse PK parameters of anti-PTK7 ADCs

| | | Tab | ADC | Payload | |
|---|---|---|---|---|---|
| | Dose | AUCₗₐₛₜ | AUCₗₐₛₜ | AUCₗₐₛₜ | |
| ADC No. | (mg/kg) | (h · μg/mL) | (h · μg/mL) | (h · μg/mL) | DAR |
| ADC5-A | 3 | 5129 | 3890 | 0.033 | 3.38 (2 h) |
| | | | | | 2.02 (168 h) |
| ADC5-1 | 3 | 4002 | 3724 | NA | 3.36 (2 h) |
| | | | | | 3.02 (168 h) |
| ADC5-2 | 3 | 3662 | 3313 | NA | 3.20 (2 h) |
| | | | | | 3.30 (168 h) |

NA: not available (because limited concentration for AUC calculation as payload concentration below quantitation limit (lowest limit of quantitation = 50 pg/mL))

### Example 5: Tumor Growth Inhibition in NCI-H1650 Xenograft Model

NCI-H1650 (ATCC) is a cell line exhibiting epithelial morphology that was isolated in 1987 from the lung tissue of a 27-year-old male smoker with stage 3 bronchoalveolar carcinoma. NCI-H1650 cells were cultured in RPMI-1640 medium supplemented with 10% (v/v) fetal bovine serum, 100 U/ml penicillin and 100 μg/mL streptomycin. On the day of implantation, cultured cells were collected and re-suspended in a cold (4° C.) mixture of PBS and Matrigel (1:1). Cell density was adjusted to 2.5×10⁷ cells/mL and the cells were placed on ice prior to inoculation.

Female BALB/c nude mice were subcutaneously injected with 5×10⁶ NCI-H1650 cells (mixed with Matrigel at a ratio of 1:1) in the right flank. After inoculation, tumor length (a) and width (b) were measured with calipers and tumor volumes were calculated using the formula V=0.5(a×b²). When tumor volumes reached approximately 200 mm³ in size, mice were randomized into 4 groups with 6 animals in each of a vehicle, ADC5-A, ADC5-1, and ADC5-2 group on Day 0. Animals were intravenously administrated with vehicle, ADC5-A (3 mg/kg), ADC5-1 (3 mg/kg), or ADC5-2 (3 mg/kg) on treatment Day 1. Whole blood was collected at timepoints of 2, 6, 48, and 168 h post-treatment from 3 mice per timepoint per group (samples at 2 and 48 h were collected from one group of 3; samples at 6 and 168 h were collected from another group of 3). Blood was centrifuged at 5000×g for 10 minutes at 4° C. to obtain serum for PK assays. Animal body weight and tumor volume were measured twice weekly. Tumor growth inhibition (TGI) was calculated using the following formula:

$$\% TGI = [ 1 - ( treated {T}_{t} - treated {T}_{0} ) / ( vehicle {T}_{t} - vehicle {T}_{0} ) ] \times 100 \%$$

treated Tₜ=mean tumor volume of a dosing group on Day t
treated T₀=mean tumor volume of a dosing group on Day 0
vehicle Tₜ=mean tumor volume of vehicle group on Day t
vehicle T₀=mean tumor volume of vehicle group on Day 0

Results of evaluating the anti-tumor effect of ADC5-A, ADC5-1, and ADC5-2 in a subcutaneous NCI-H1650 xenograft model are presented in Table 18 and FIG. 8. Data is presented as mean tumor volume±standard error of the mean (SEM). Single dose treatment with 3 mg/kg ADC5-A, ADC5-1, and ADC5-2 exhibited anti-tumor activity with a TGI of 65% (adjusted p=0.0450), 83% (adjusted p=0.0004), and 84% (adjusted p=0.0011), respectively, on treatment Day 18. ADC5-1 and ADC5-2 showed better efficacy than did ADC5-A at 3 mg/kg. ADCs in all treatment groups were well tolerated and animals in all treatment groups showed no significant body weight decrease or abnormal clinical observation.

TABLE 18
TGI measurement of ADC5-A, ADC5-1,
and ADC5-2 on treatment Day 18

| ADC | TGI (%) on D 18 |
|---|---|
| ADC5-A, 3 mg/kg | 65 |
| ADC5-1, 3 mg/kg | 83 |
| ADC5-2, 3 mg/kg | 84 |

Although the foregoing disclosure has been presented in some detail by way of illustration and example for purposes of clarity of understanding, it is apparent to those skilled in the art that certain minor changes and modifications will be practiced. Therefore, the description and examples should not be construed as limiting.

It is to be understood that, if any prior art publication is referred to herein, such reference does not constitute an admission that the publication forms a part of the common general knowledge in the art in any country.

The disclosures of all non-patent publications, patents, patent applications, and published patent applications referred to herein by an identifying citation are hereby incorporated herein by reference in their entireties.

What is claimed is:

1. A compound of formula (I): 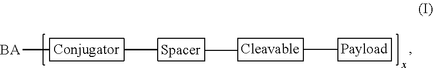
or a pharmaceutically acceptable salt, tautomer, solvate, or stereoisomer thereof, wherein:
BA is a binding agent selected from a humanized, monoclonal, chimeric, or human antibody or an antigen-binding fragment thereof;
Conjugator has formula (II) or (III): 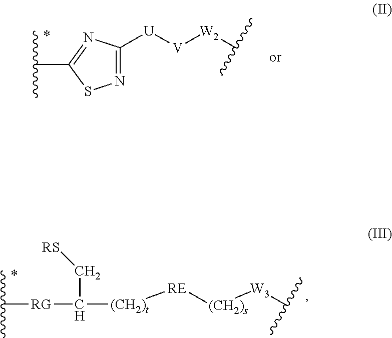
wherein:
U is a bond, heteroarylene, or arylene;
V is a bond or —C≡C—(CH₂)ₙ—;
n is an integer from 0 to 10 inclusive;
W₂ is —C(═O)—, —NH—, or —O—;
RG is 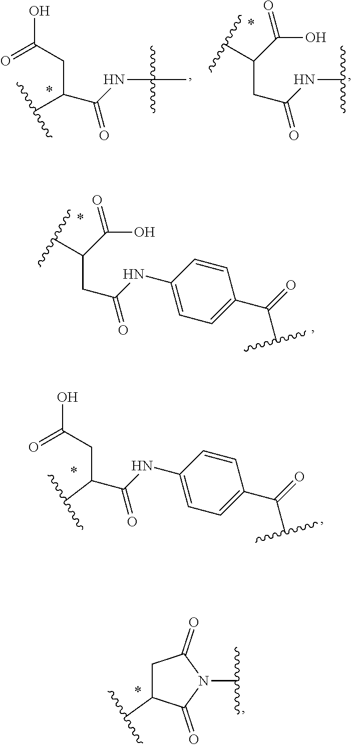 [SMILES: *NC(=O)C(*)CC(=O)O ; *NC(=O)CC(*)C(=O)O ; *C(=O)c1ccc(NC(=O)CC(*)C(=O)O)cc1 ; *C(=O)c1ccc(NC(=O)C(*)CC(=O)O)cc1 ; *C1CC(=O)N(*)C1=O]
-(succinimid-3-yl-N)—, or 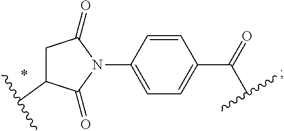 [SMILES: *C(=O)c1ccc(N2C(=O)CC(*)C2=O)cc1]
RS is —NR¹ªR¹;
each of R^{1a} and R^{1b} is, independently, H or substituted or unsubstituted C₁₋₄ alkyl;
RE is a bond, —O—, —OC(═O)—, —OC(═O)NR⁶—, —NHC(═O)NR⁶—, —OS(═O)₂NR⁶—, —NHS(═O)₂NR⁶—, or —OC(═O)NHS(═O)₂NR⁶—;
R⁶ is H or substituted or unsubstituted C₁₋₄ alkyl;
W₃ is —C(═O)—, —NH—, or —O—;
each of s and t is, independently, 0, 1, or 2; [symbol]
indicates a point of covalent attachment within the compound, and
* marks the bond where Conjugator connects to BA;
Spacer is a bond, **—NH—(CH₂CH₂O)ₘ—CH₂CH₂—C(═O)—, *—(CH₂)ₘ—C(═O)—, **—CH₂—C(═O)—NH—(CH₂)ₘ—C(═O)—, *—(CH₂CH₂O)ₘ—CH₂CH₂—C(═O)—, **—CH[—(CH₂)ₘ—COOH]—C(═O)—, **—CH₂—C(═O)—NH—(CH₂)ₘ—C(═O)—NH—(CH₂)ₘ—C(═O)—, *—C(═O)—(CH₂)ₘ—C(═O)—, *—C(═O)—(CH₂CH₂O)ₘ—CH₂CH₂—NH—, **—NH—(CH₂)ₘ—C(═O)—, or **—NH—(CH₂)ₘ—O—, wherein:
m is an integer from 1 to 12 inclusive, and
** marks the bond where Spacer connects to Conjugator;
Cleavable has formula (IVa), (IVb), (IVc), (Va), (Vb), (VIa), (VIb), (VIIa), or (VIIb): 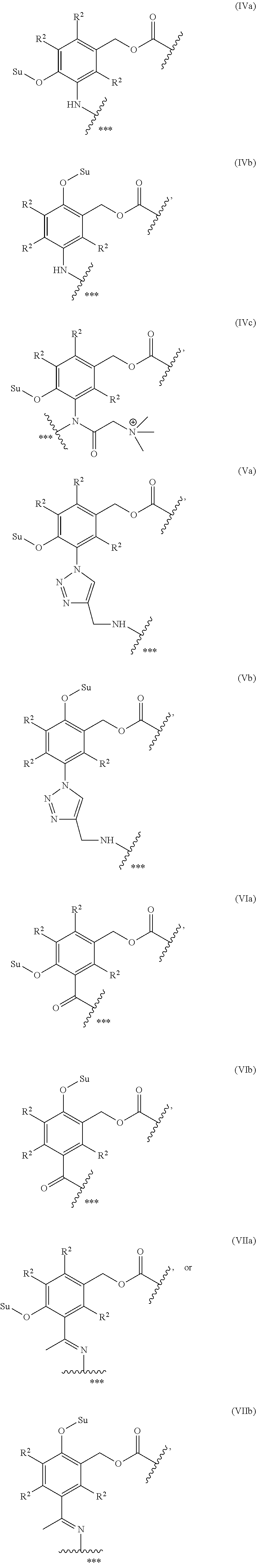
wherein:
Su is a Sugar moiety,
each R² is, independently, hydrogen, halogen, substituted or unsubstituted C₁₋₄ alkyl, —CN, or —NO₂, and
*** marks the bond where Cleavable connects to Spacer;
Payload is a payload residue; and
x is from 1 to 15 inclusive.

2. The compound of claim 1, wherein Conjugator has formula (II).

3. The compound of claim 2, wherein U is arylene.

4. The compound of claim 3, wherein U is phenylene.

5. The compound of claim 4, wherein U is

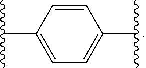 [SMILES: *c1ccc(*)cc1]

6. The compound of claim 2, wherein U is heteroarylene.

7. The compound of claim 6, wherein U is a bivalent pyrimidine ring.

8. The compound of claim 7, wherein U is

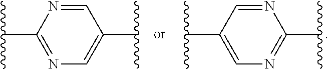 [SMILES: *c1cnc(*)nc1 ; *c1cnc(*)nc1]

9. The compound of claim 2, wherein U is a bond.

10. The compound of any one of claims 1-9, wherein V is a bond.

11. The compound of any one of claims 1-9, wherein V is —C≡C—(CH₂)ₙ—.

12. The compound of claim 11, wherein V is —C≡C—(CH₂)₃—.

13. The compound of any one of claims 1-12, wherein W₂ is —C(═O)—.

14. The compound of claim 1, wherein Conjugator has formula (III).

15. The compound of claim 14, wherein RS is —NH₂ or —N(CH₃)₂.

16. The compound of claim 15, wherein RS is —NH₂.

17. The compound of any one of claims 14-16, wherein RE is —OC(═O)NH—.

18. The compound of any one of claims 14-17, wherein RG is

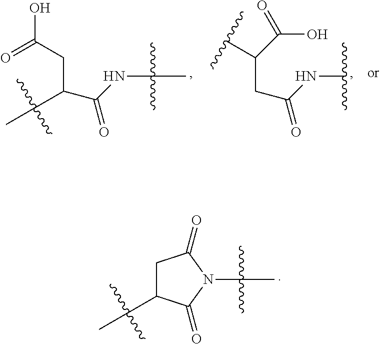 [SMILES: *NC(=O)C(*)CC(=O)O ; *NC(=O)CC(*)C(=O)O ; *C1CC(=O)N(*)C1=O]

19. The compound of any one of claims 14-18, wherein each of s and t is 2.

20. The compound of any one of claims 14-19, wherein W₃ is —C(═O)— or —NH—.

21. The compound of any one of claims 1-20, wherein Spacer is a bond, **—NH—(CH₂CH₂O)ₘ—CH₂CH₂—C(═O)—, or **—NH—(CH₂)ₘ—O—.

22. The compound of any one of claims 1-21, wherein m is 2, 4, 6, or 8.

23. The compound of claim 1, wherein Conjugator has formula (IIa1), (IIa2), (IIa3), or (IIIa):

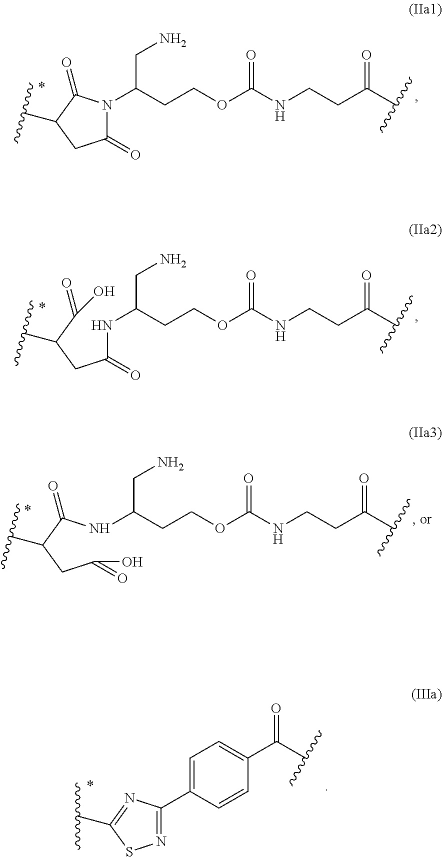 [SMILES: *C(=O)CCNC(=O)OCCC(CN)N1C(=O)CC(*)C1=O ; *C(=O)CCNC(=O)OCCC(CN)NC(=O)CC(*)C(=O)O ; *C(=O)CCNC(=O)OCCC(CN)NC(=O)C(*)CC(=O)O ; *C(=O)c1ccc(-c2nsc(*)n2)cc1]

24. The compound of any one of claims 1-23, wherein Cleavable has formula (IVa1), (IVa2), (IVa3), (Va1), (VIa1), or (VIIa1):

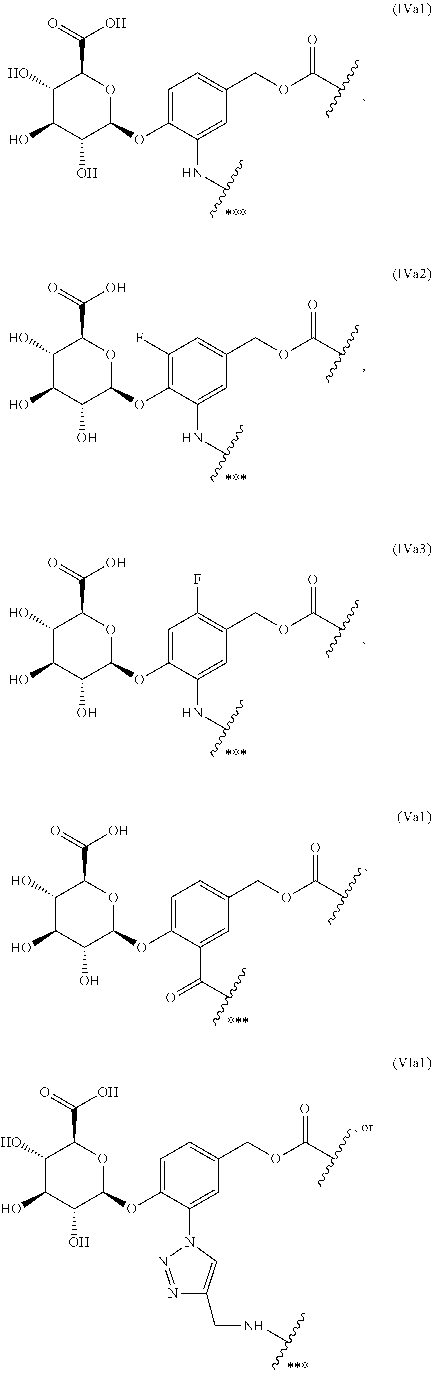 [SMILES: *Nc1cc(COC(*)=O)ccc1O[C@@H]1O[C@H](C(=O)O)[C@@H](O)[C@H](O)[C@H]1O ; *Nc1cc(COC(*)=O)cc(F)c1O[C@@H]1O[C@H](C(=O)O)[C@@H](O)[C@H](O)[C@H]1O ; *Nc1cc(COC(*)=O)c(F)cc1O[C@@H]1O[C@H](C(=O)O)[C@@H](O)[C@H](O)[C@H]1O ; *C(=O)OCc1ccc(O[C@@H]2O[C@H](C(=O)O)[C@@H](O)[C@H](O)[C@H]2O)c(C(*)=O)c1 ; *NCc1cn(-c2cc(COC(*)=O)ccc2O[C@@H]2O[C@H](C(=O)O)[C@@H](O)[C@H](O)[C@H]2O)nn1]

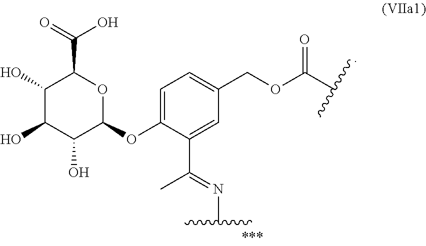 [SMILES: */N=C(\C)c1cc(COC(*)=O)ccc1O[C@@H]1O[C@H](C(=O)O)[C@@H](O)[C@H](O)[C@H]1O]

25. The compound of any one of claims 1-24, wherein Payload is a residue of one of the following formulas.

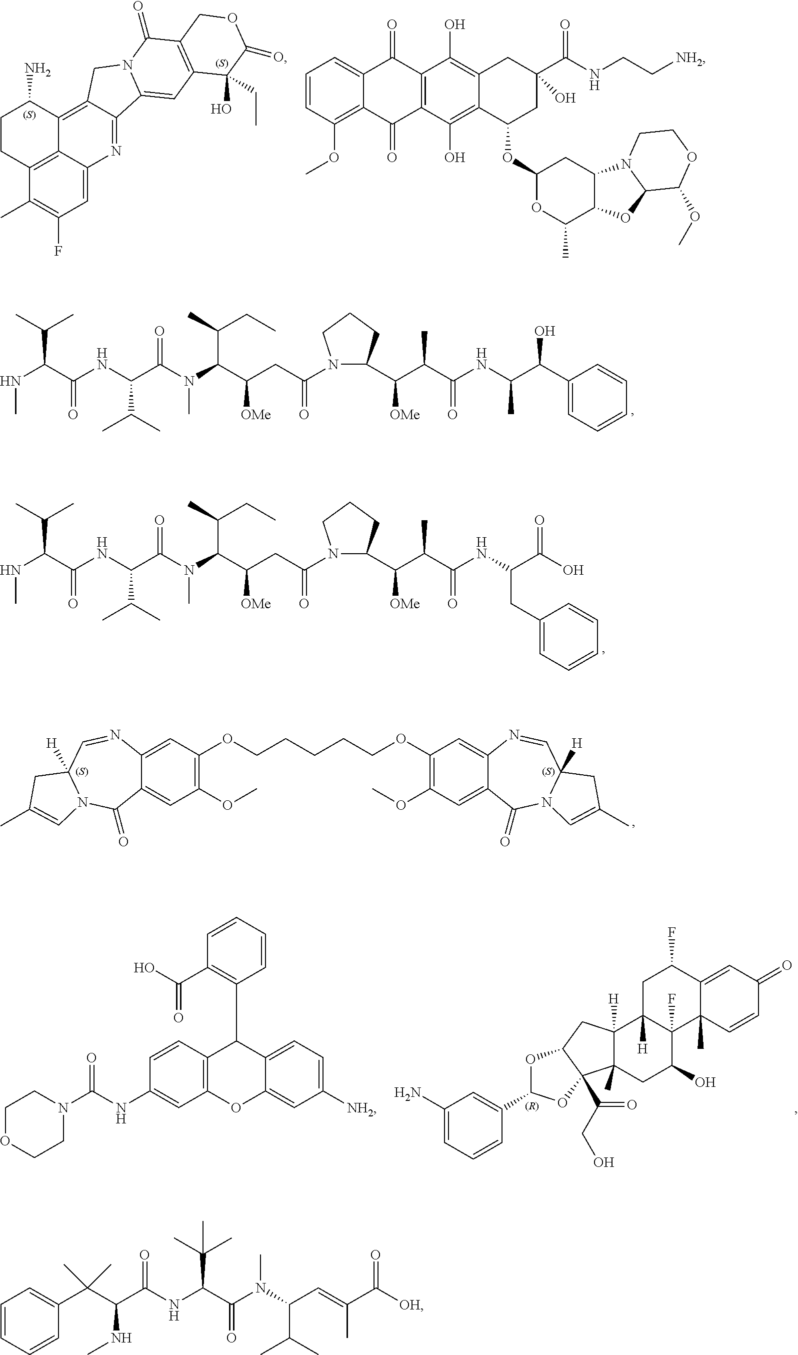

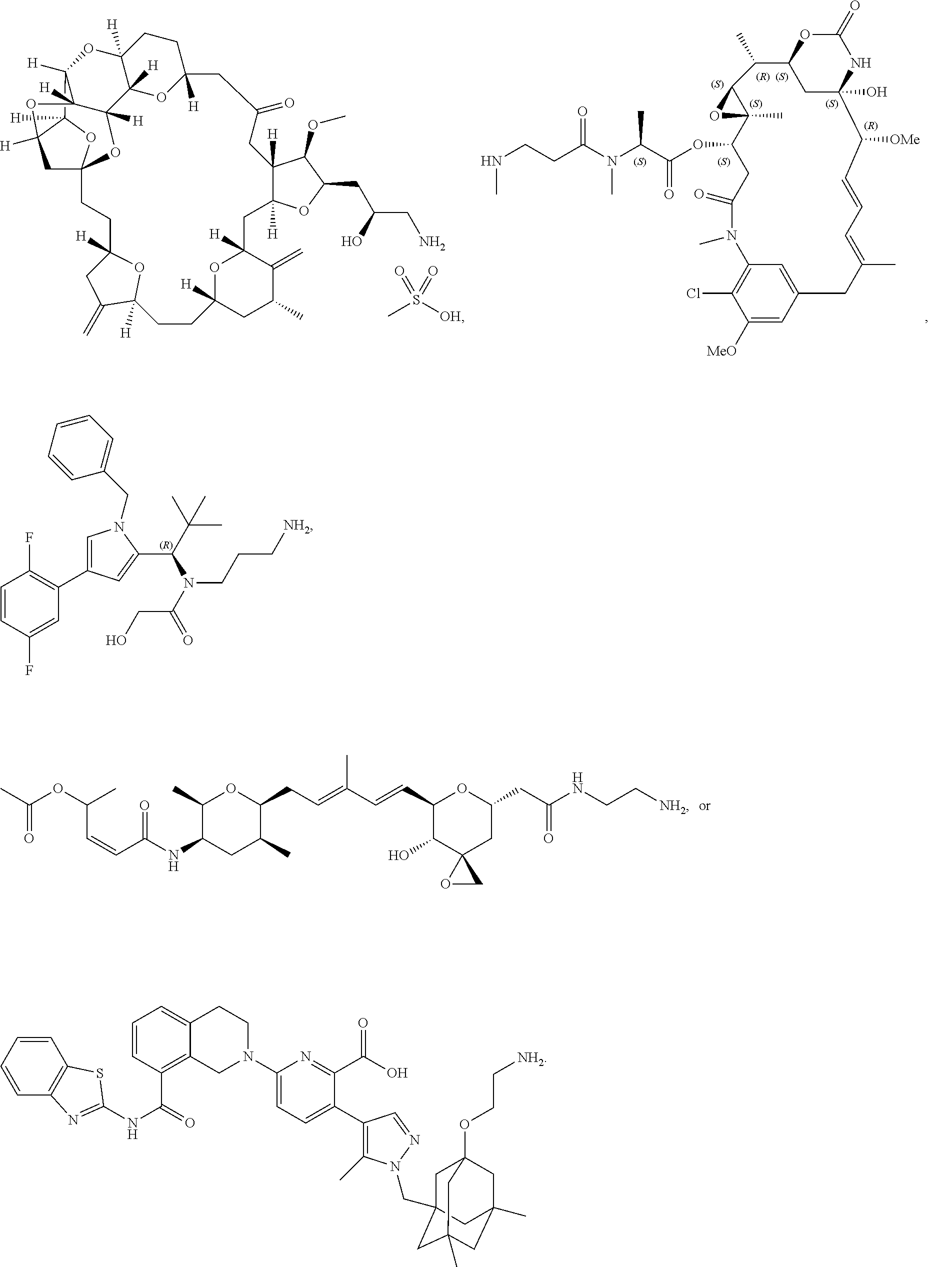

26. The compound of any one of claims 1-25, wherein Payload is

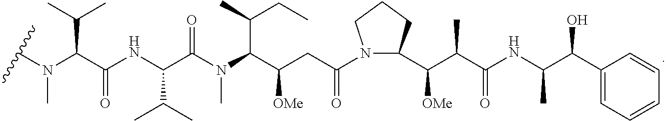 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

27. The compound of any one of claims 1-26, wherein BA binds to one or more receptors selected from B7H3, PTK7, or CLL1.

28. The compound of any one of claims 1-27, wherein BA is ifinatamab, 6E7, or cofetuzumab, or an antigen-binding fragment of ifinatamab, 6E7, or cofetuzumab.

29. The compound of claim 1, wherein the compound is 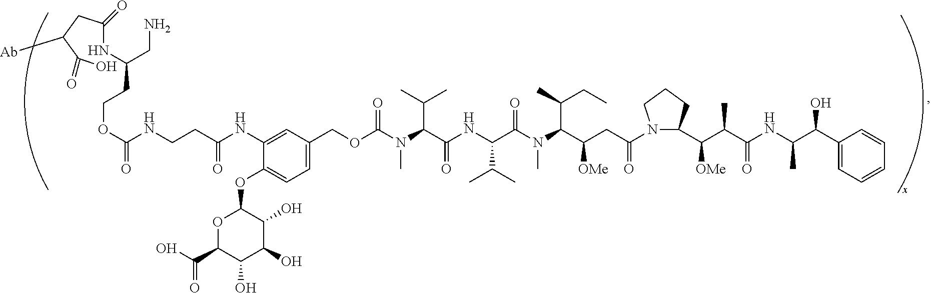
wherein Ab is 6E7 or an antigen-binding fragment thereof; 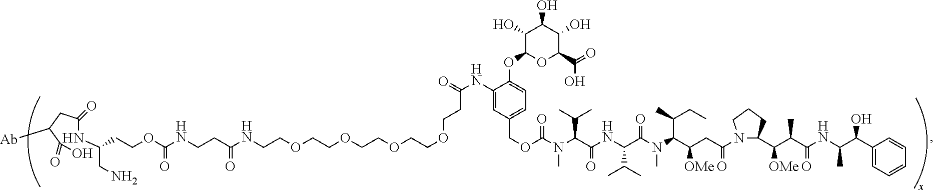
wherein Ab is 6E7 or an antigen-binding fragment thereof; 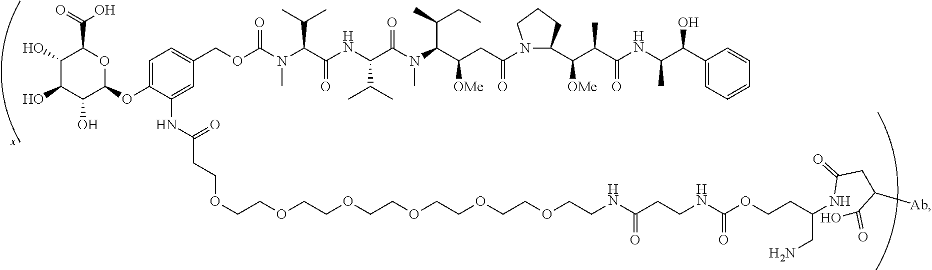
wherein Ab is 6E7 or an antigen-binding fragment thereof; 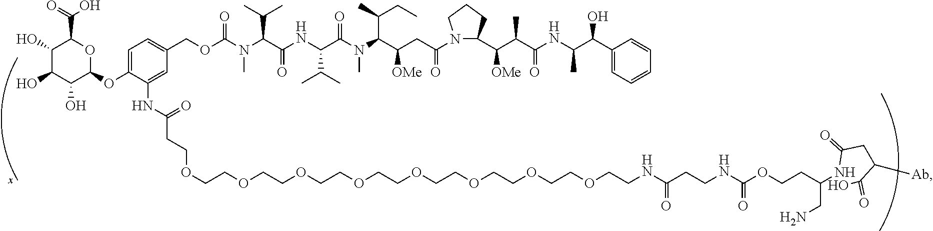
wherein Ab is 6E7 or an antigen-binding fragment thereof; 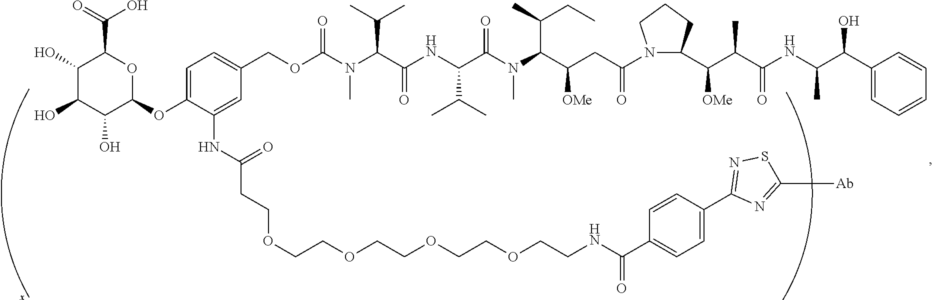
wherein Ab is 6E7 or an antigen-binding fragment thereof; 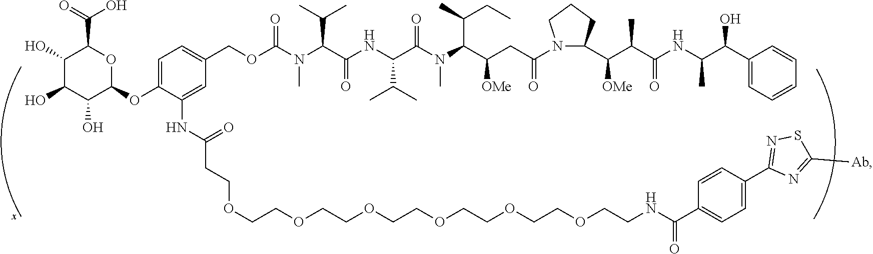
wherein Ab is 6E7 or an antigen-binding fragment thereof; 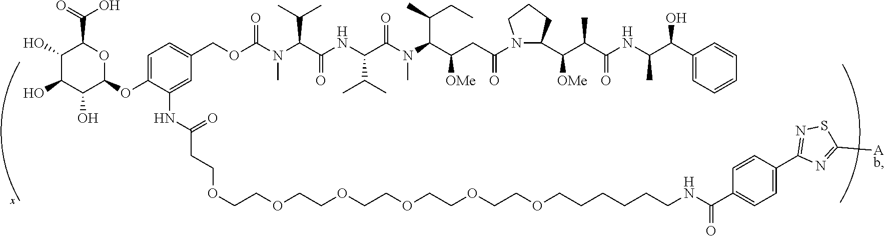
wherein Ab is 6E7 or an antigen-binding fragment thereof; 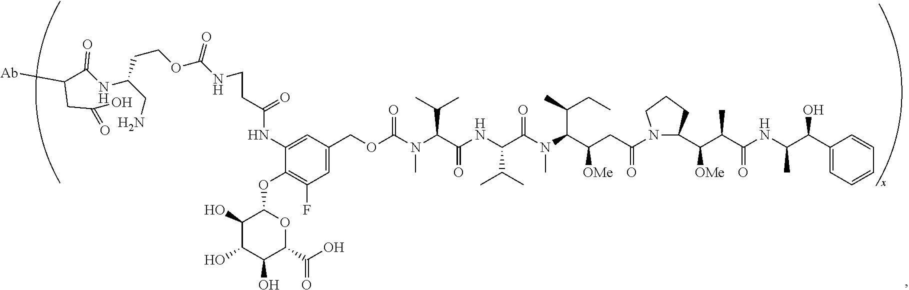
wherein Ab is 6E7 or an antigen-binding fragment thereof; 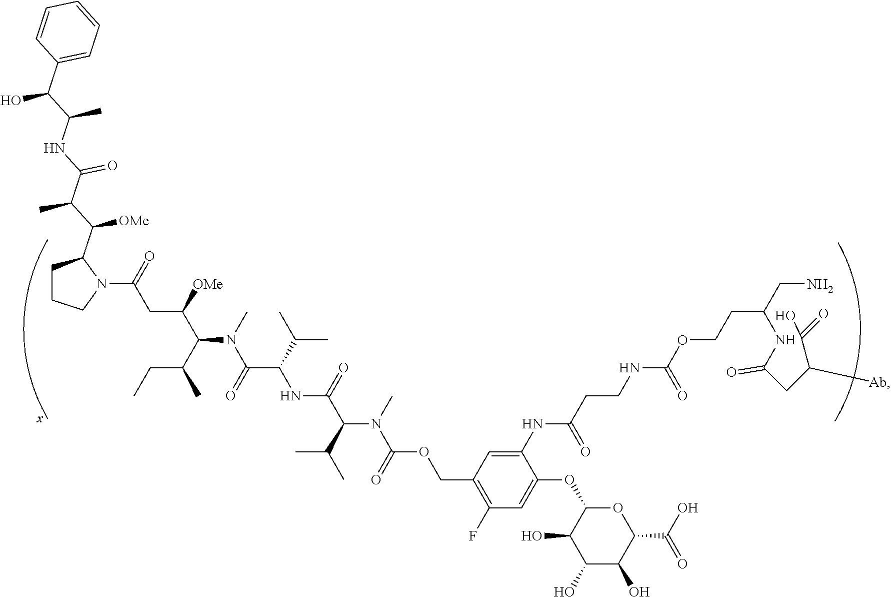
wherein Ab is 6E7 or an antigen-binding fragment thereof; 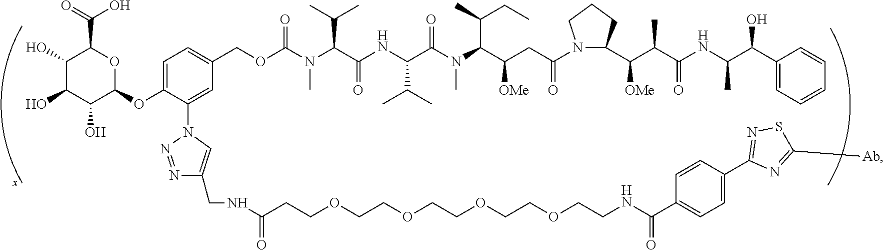
wherein Ab is 6E7 or an antigen-binding fragment thereof; 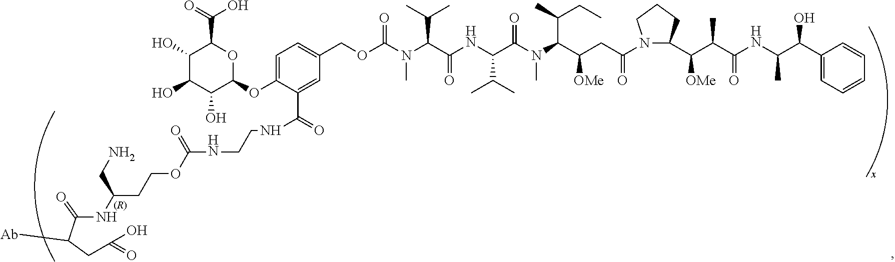
wherein Ab is 6E7 or an antigen-binding fragment thereof; 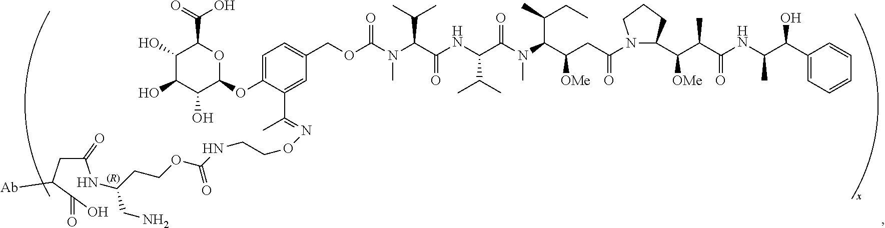
wherein Ab is 6E7 or an antigen-binding fragment thereof; 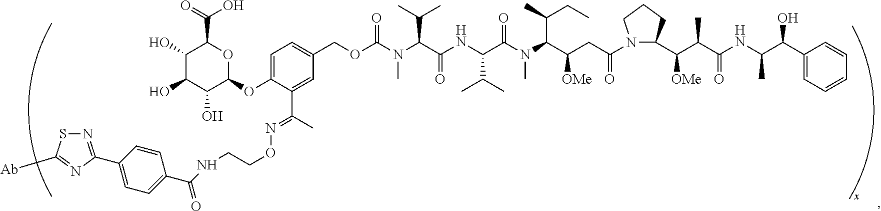
wherein Ab is 6E7 or an antigen-binding fragment thereof; 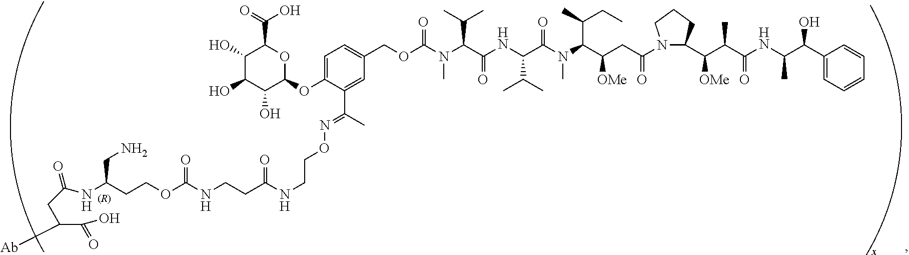
wherein Ab is 6E7 or an antigen-binding fragment thereof; or 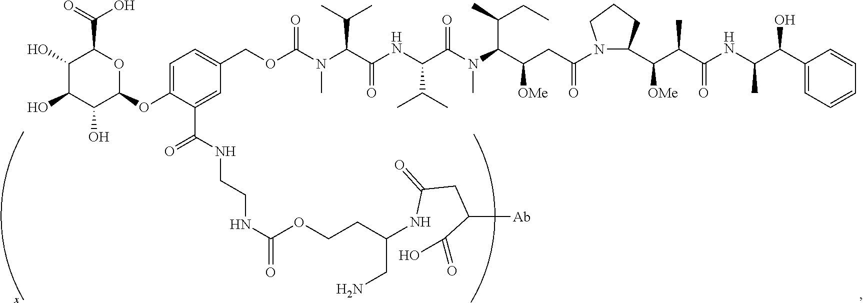
wherein Ab is ifinatamab or an antigen-binding fragment thereof,
or a pharmaceutically acceptable salt, tautomer, solvate, or stereoisomer of any of the foregoing.

30. The compound of any one of claims 1-29, wherein x is about 3.5 to about 4.5.

31. The compound of claim 30, wherein the compound is 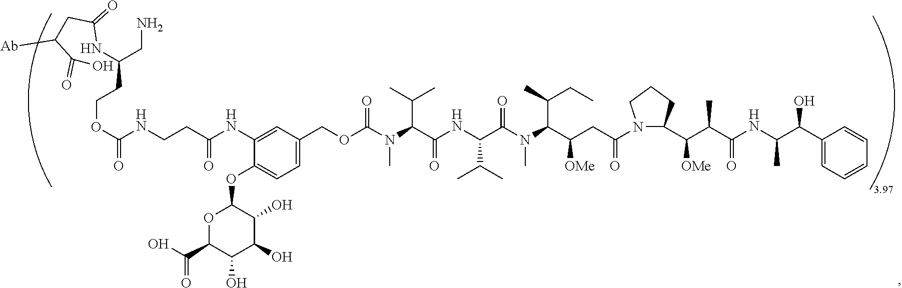
wherein Ab is 6E7 or an antigen-binding fragment thereof; 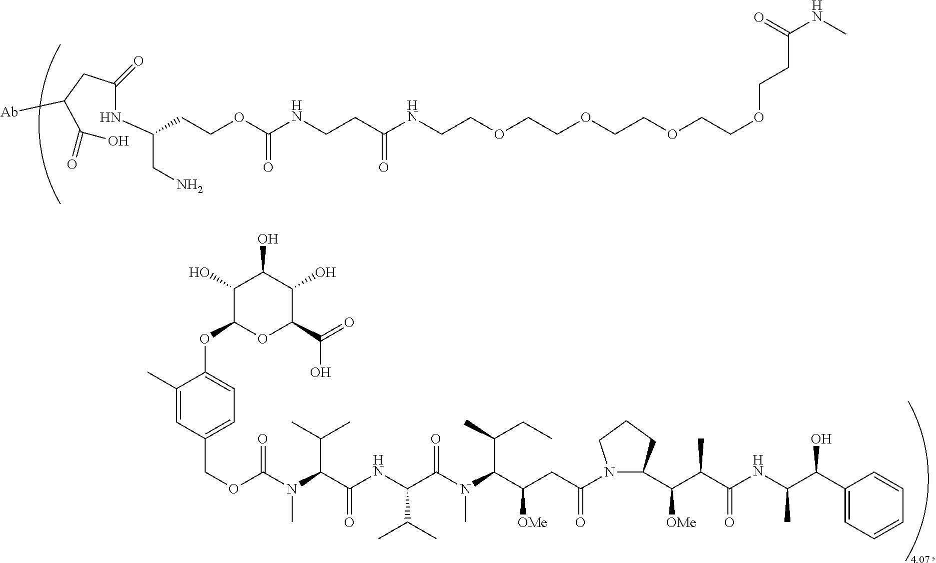
wherein Ab is 6E7 or an antigen-binding fragment thereof; 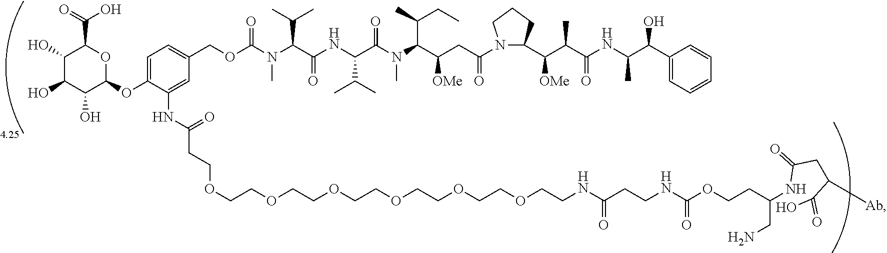
wherein Ab is 6E7 or an antigen-binding fragment thereof; 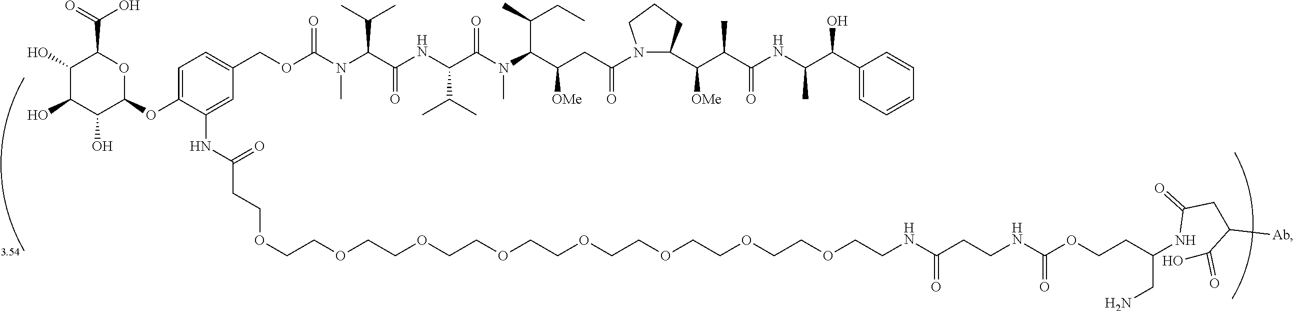
wherein Ab is 6E7 or an antigen-binding fragment thereof; 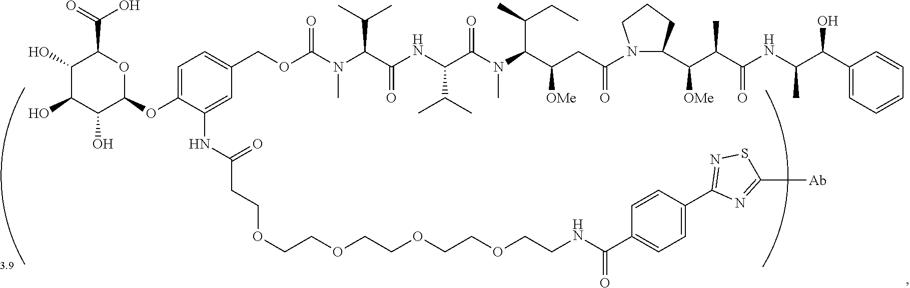
wherein Ab is 6E7 or an antigen-binding fragment thereof; 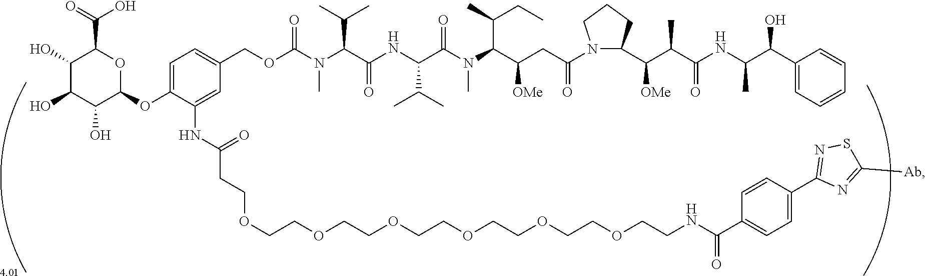
wherein Ab is 6E7 or an antigen-binding fragment thereof; 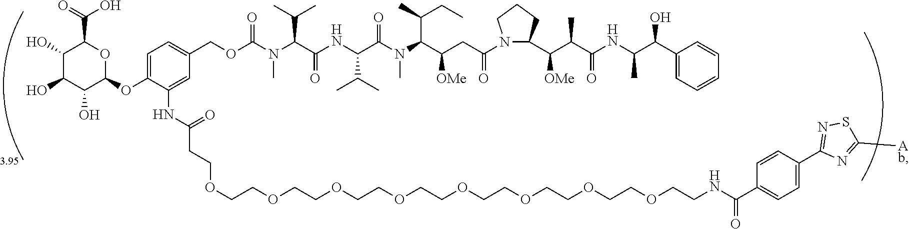
wherein Ab is 6E7 or an antigen-binding fragment thereof; 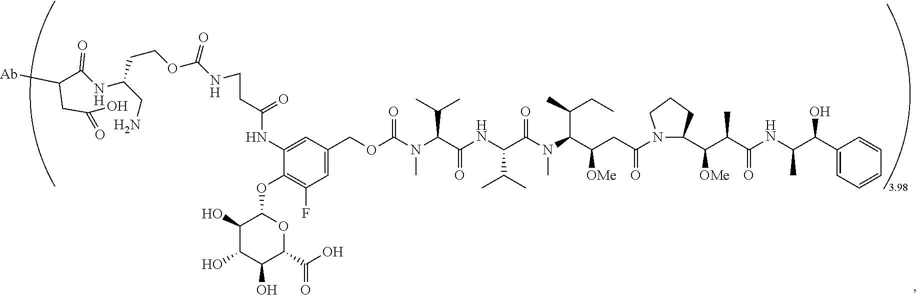
wherein Ab is 6E7 or an antigen-binding fragment thereof; 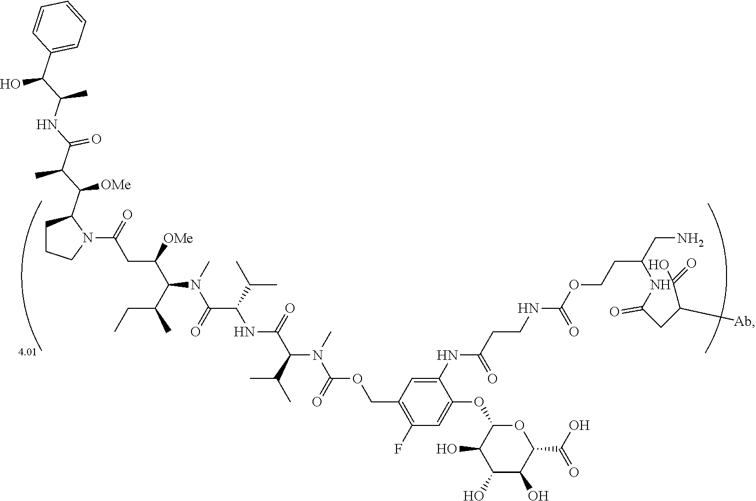
wherein Ab is 6E7 or an antigen-binding fragment thereof; 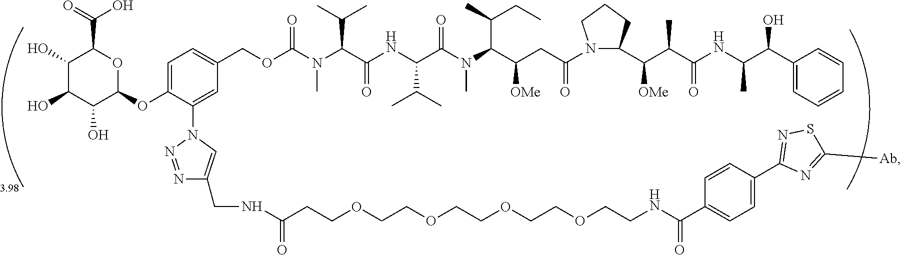
wherein Ab is 6E7 or an antigen-binding fragment thereof; 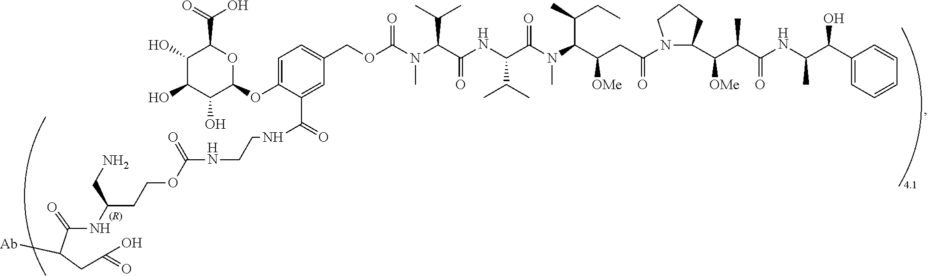
wherein Ab is 6E7 or an antigen-binding fragment thereof; 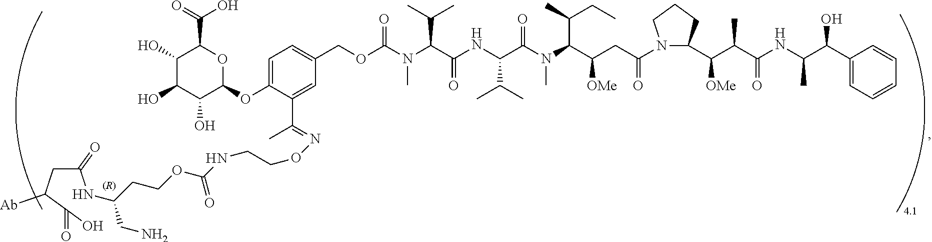
wherein Ab is 6E7 or an antigen-binding fragment thereof; 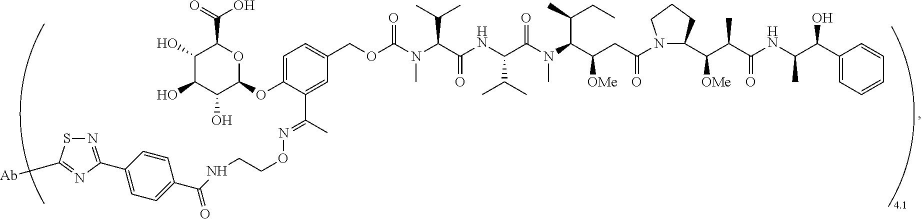
wherein Ab is 6E7 or an antigen-binding fragment thereof; 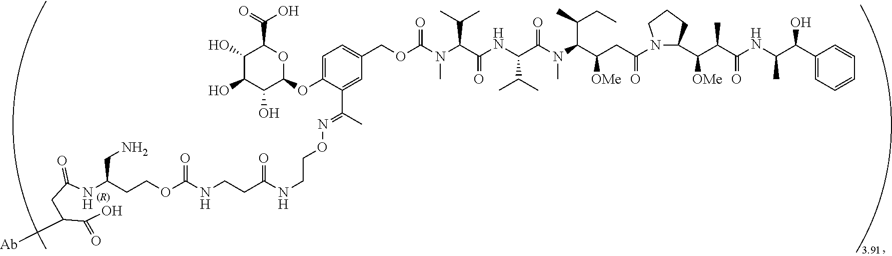
wherein Ab is 6E7 or an antigen-binding fragment thereof; or 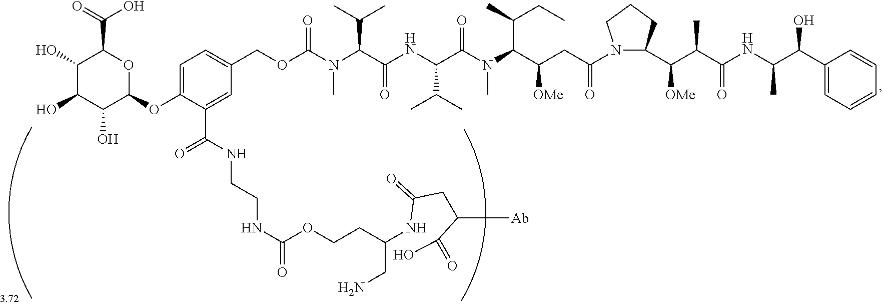
wherein Ab is ifinatamab or an antigen-binding fragment thereof,
or a pharmaceutically acceptable salt, tautomer, solvate, or stereoisomer of any of the foregoing.

32. A compound of formula (XI): 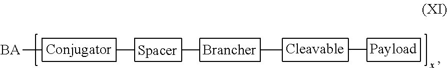
or a pharmaceutically acceptable salt, tautomer, solvate, or stereoisomer thereof, wherein:
BA is a binding agent selected from a humanized, monoclonal, chimeric, or human antibody or an antigen-binding fragment thereof;
Conjugator has formula (II) or (III): 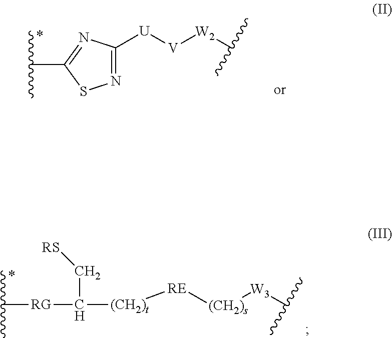
wherein:
U is a bond, heteroarylene, or arylene;
V is a bond or —C≡C—(CH₂)ₙ—;
n is an integer from 0 to 10 inclusive;
W₂ is —C(═O)—, —NH—, or —O—;
RG is 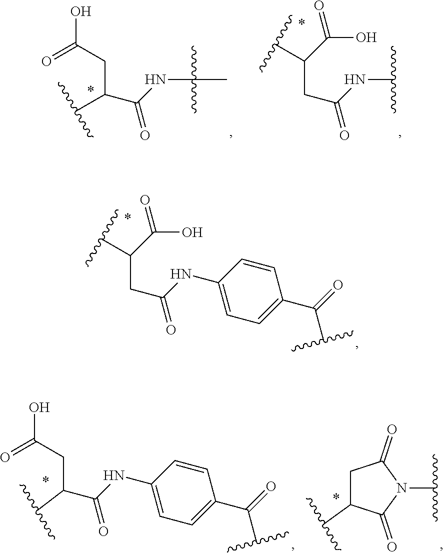 [SMILES: *NC(=O)C(*)CC(=O)O ; *NC(=O)CC(*)C(=O)O ; *C(=O)c1ccc(NC(=O)CC(*)C(=O)O)cc1 ; *C(=O)c1ccc(NC(=O)C(*)CC(=O)O)cc1 ; *C1CC(=O)N(*)C1=O]
-(succinimid-3-yl-N)—, or 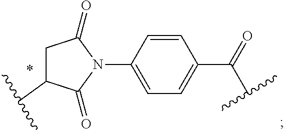 [SMILES: *C(=O)c1ccc(N2C(=O)CC(*)C2=O)cc1]
RS is —NR^{1a}R^{1b};
each of R^{1a} and R^{1b} is, independently, H or substituted or unsubstituted C₁₋₄ alkyl;
RE is a bond, —O—, —OC(═O)—, —OC(═O)NR⁶—, —NHC(═O)NR⁶—, —OS(═O)₂NR⁶—, —NHS(═O)₂NR⁶—, or —OC(═O)NHS(═O)₂NR⁶—;
R⁶ is H or substituted or unsubstituted C₁₋₄ alkyl;
W₃ is —C(═O)—, —NH—, or —O—;
each of s and t is, independently, 0, 1, or 2; [symbol]
indicates a point of covalent attachment within the compound, and
* marks the bond where Conjugator connects to BA;
Spacer is a bond, **—NH—(CH₂CH₂O)ₘ—CH₂CH₂—C(═O)—, **—C(═O)—(CH₂CH₂O)ₘ—CH₂CH₂—NH—, **—(CH₂)ₘ—C(═O)—, **—CH₂—C(═O)—NH—(CH₂)ₘ—C(═O)—, **—(CH₂CH₂O)ₘ—CH₂CH₂—C(═O)—, **—C(═O)—(CH₂CH₂O)ₘ—CH₂CH₂—NH—, **—CH[—(CH₂)ₘ—COOH]—C(═O)—, *—CH₂—C(═O)—NH—(CH₂)ₘ—C(═O)—NH—(CH₂)ₘ—C(═O)—, **—C(═O)—(CH₂)ₘ—C(═O)—, **—NH—(CH₂)ₘ—C(═O)—, or **—NH—(CH₂)ₘ—O—, wherein:
m is an integer from 1 to 12 inclusive, and
** marks the bond where Spacer connects to Conjugator;
Cleavable has formula (IVa′), (IVb′), (IVc′), (Va′), (Vb′), (VIa′), (VIb′), (VIIa′), or (VIIb′): 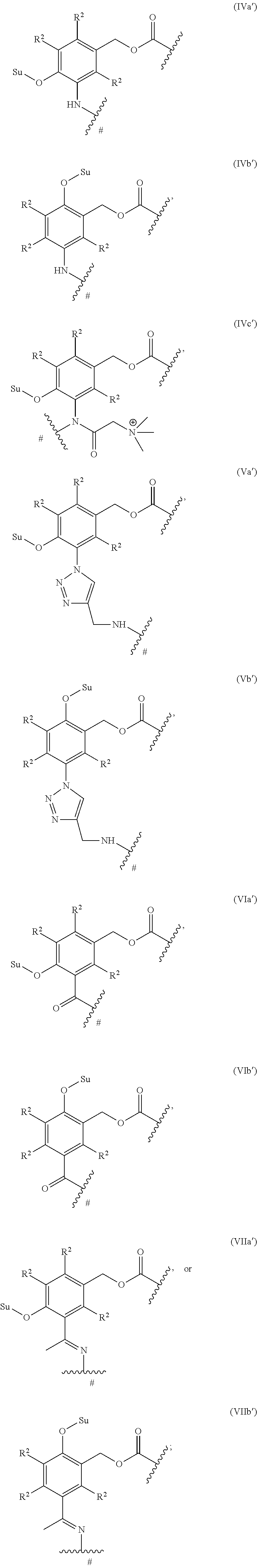
wherein:
Su is a Sugar moiety,
each R² is, independently, hydrogen, halogen, substituted or unsubstituted C₁₋₄ alkyl, —CN, or —NO₂, and
#marks the bond where Cleavable connects to Brancher,
Brancher has formula (XIIa), (XIIb), (XIIc), or (XIId): 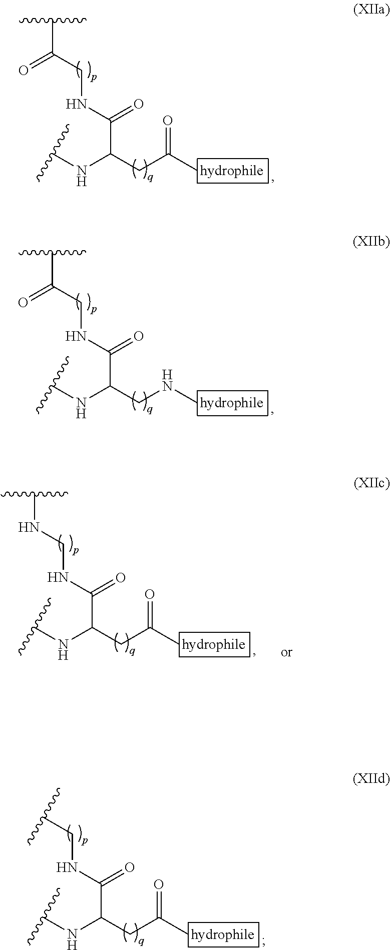
wherein:
each of p and q is, independently, 1, 2, 3, or 4;
hydrophile is —NH—(CH₂CH₂O)ₐ—(CH₂)_{b}CH₃, —N(CH₃)—((CH₂)_{c}C(═O) N(CH₃))ₐ—(CH₂)_{c}C(═O) NH₂, —C(═O)—(CH₂CH₂O)ₐ—(CH₂)_{b}CH₃, —C(═O)—(CH₂CH₂O)ₐ—(CH₂)_{b}NHC(═O) (CH₂)₃N(CH₃)₂ (CH₂)₃S(═O)₂OH, —NH—(CH₂CH₂O)ₐ—(CH₂)_{b}NHC(═O)(CH₂)₃N(CH₃)₂ (CH₂)₃S(═O)₂OH, 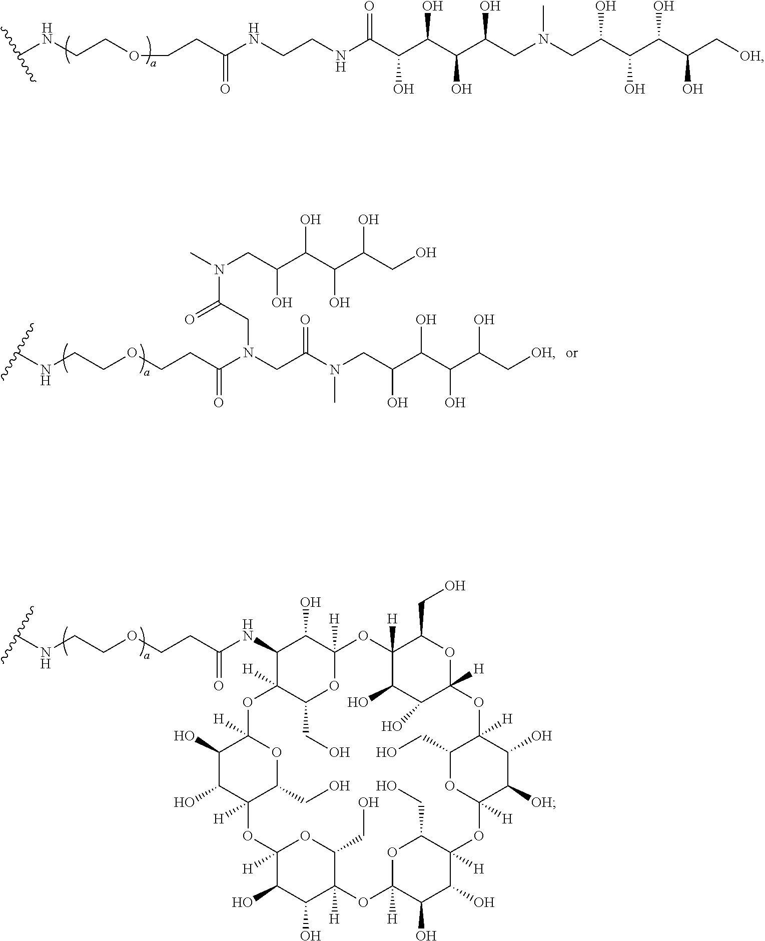
a is an integer from 1 to 18 inclusive;
b is 0, 1, or 2, and
c is 1, 2, 3, or 4;
Payload is a payload residue; and
x is from 1 to 15 inclusive.

33. The compound of claim 32, wherein Conjugator has formula (II).

34. The compound of claim 33, wherein U is arylene.

35. The compound of claim 34, wherein U is phenylene.

36. The compound of claim 35, wherein U is

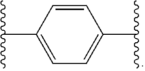 [SMILES: *c1ccc(*)cc1]

37. The compound of claim 32, wherein U is heteroarylene.

38. The compound of claim 37, wherein U is a bivalent pyrimidine ring.

39. The compound of claim 38, wherein U is

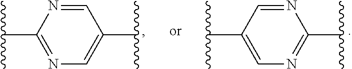 [SMILES: *c1cnc(*)nc1 ; *c1cnc(*)nc1]

40. The compound of claim 32, wherein U is a bond.

41. The compound of any one of claims 32-40, wherein V is a bond.

42. The compound of any one of claims 32-40, wherein V is —C≡C—(CH₂)ₙ—.

43. The compound of claim 42, wherein V is —C≡C—(CH₂)₃—.

44. The compound of any one of claims 32-43, wherein W₂ is —C(═O)—.

45. The compound of claim 32, wherein Conjugator has formula (III).

46. The compound of claim 45, wherein RS is —NH₂ or —N(CH₃)₂.

47. The compound of claim 46, wherein RS is —NH₂.

48. The compound of any one of claims 45-47, wherein RE is —OC(═O) NH—.

49. The compound of any one of claims 45-48, wherein RG is

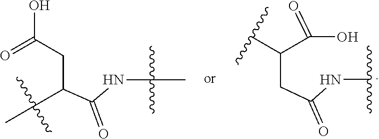 [SMILES: *NC(=O)C(*)CC(=O)O ; *NC(=O)CC(*)C(=O)O]

50. The compound of any one of claims 45-49, wherein each of s and t is, independently, 0 or 2.

51. The compound of any one of claims 45-50, wherein W₃ is —C(═O)—.

52. The compound of any one of claims 32-51, wherein Spacer is a bond.

53. The compound of claim 32, wherein Conjugator has formula (IIa1), (IIa2), (IIa3), (IIa4), (IIa5), or (IIIa):

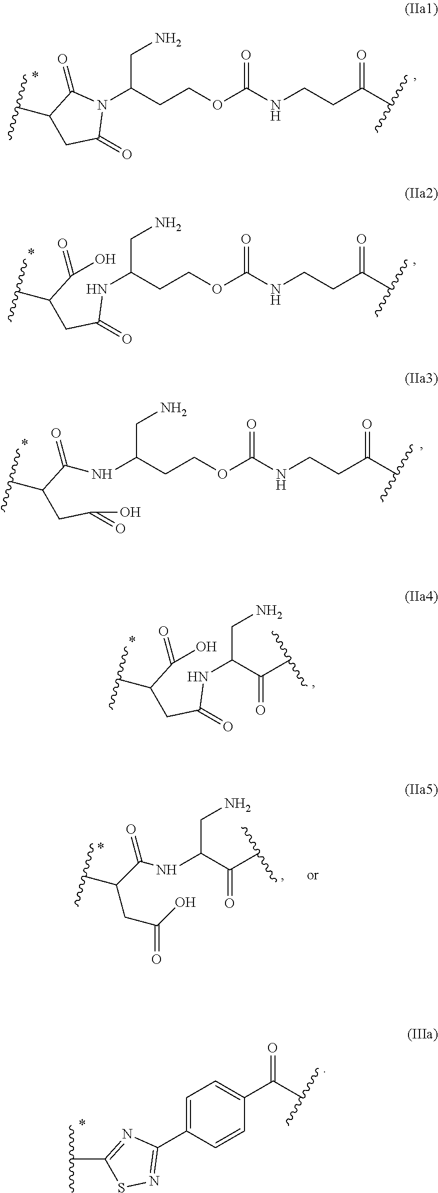 [SMILES: *C(=O)CCNC(=O)OCCC(CN)N1C(=O)CC(*)C1=O ; *C(=O)CCNC(=O)OCCC(CN)NC(=O)CC(*)C(=O)O ; *C(=O)CCNC(=O)OCCC(CN)NC(=O)C(*)CC(=O)O ; *C(=O)C(CN)NC(=O)CC(*)C(=O)O ; *C(=O)C(CN)NC(=O)C(*)CC(=O)O ; *C(=O)c1ccc(-c2nsc(*)n2)cc1]

54. The compound of any one of claims 32-53, wherein Cleavable has formula (IVa′1), (IVc′1), or (Va′1):

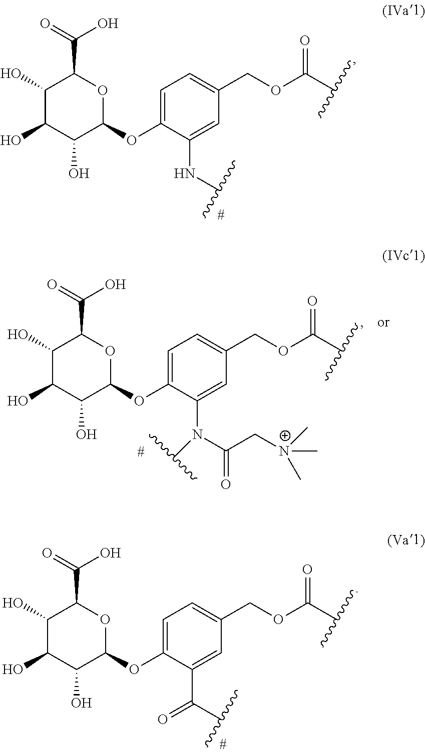 [SMILES: *Nc1cc(COC(*)=O)ccc1O[C@@H]1O[C@H](C(=O)O)[C@@H](O)[C@H](O)[C@H]1O ; *C(=O)OCc1ccc(O[C@@H]2O[C@H](C(=O)O)[C@@H](O)[C@H](O)[C@H]2O)c(N(*)C(=O)C[N+](C)(C)C)c1 ; *C(=O)OCc1ccc(O[C@@H]2O[C@H](C(=O)O)[C@@H](O)[C@H](O)[C@H]2O)c(C(*)=O)c1]

55. The compound of any one of claims 32-54, wherein hydrophile is —NH—(CH₂CH₂O)ₐ—(CH₂)_{b}CH₃, —N(CH₃)—((CH₂)_{c}C(═O) N(CH₃))ₐ—(CH₂)_{c}C(═O) NH₂, —C(═O)—(CH₂CH₂O)ₐ—(CH₂)_{b}CH₃, —C(═O)—(CH₂CH₂O)ₐ—(CH₂)_{b}NHC(═O)(CH₂)₃N(CH₃)₂ (CH₂)₃S(═O)₂OH,

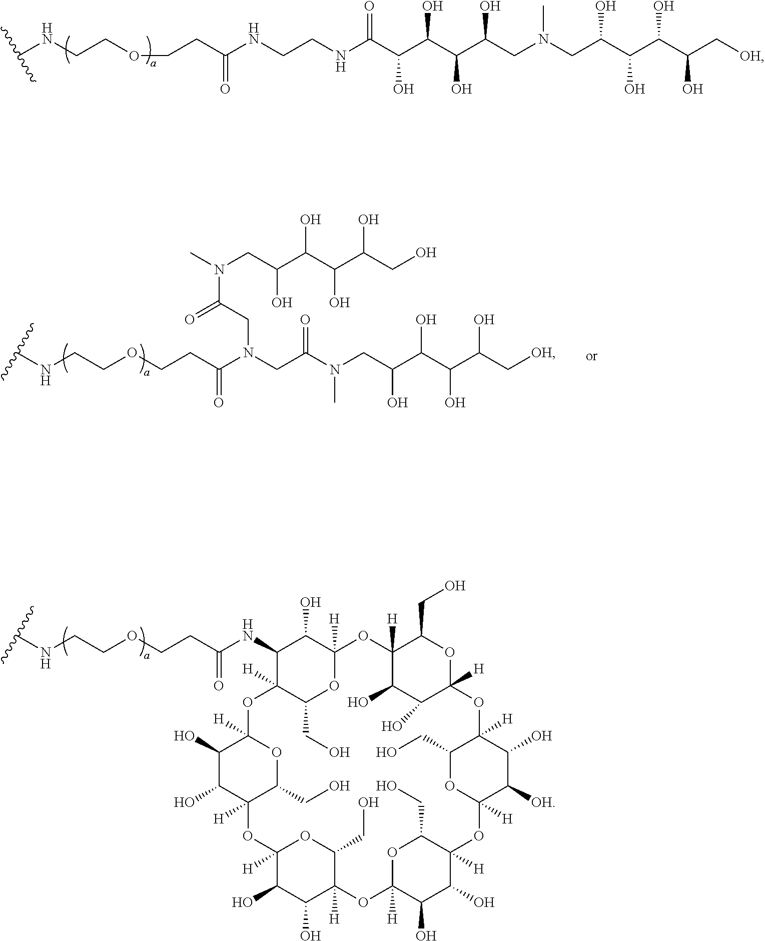

56. The compound of any one of claims 32-55, wherein a is 4, 8, 11, or 12.

57. The compound of any one of claims 32-56, wherein b is 0 or 2.

58. The compound of any one of claims 32-57, wherein c is 1.

59. The compound of claim 55, wherein hydrophile is —NH—(CH₂CH₂O)₁₂—CH₃, —N(CH₃)—((CH₂) C(═O) N(CH₃))₁₁—(CH₂)_{c}C(═O) NH₂, —C(═O)—(CH₂CH₂O)₁₂—CH₃, —C(═O)—(CH₂CH₂O)₈—(CH₂)₂NHC(═O)(CH₂)₃N(CH₃)₂ (CH₂)₃S(═O)₂OH,

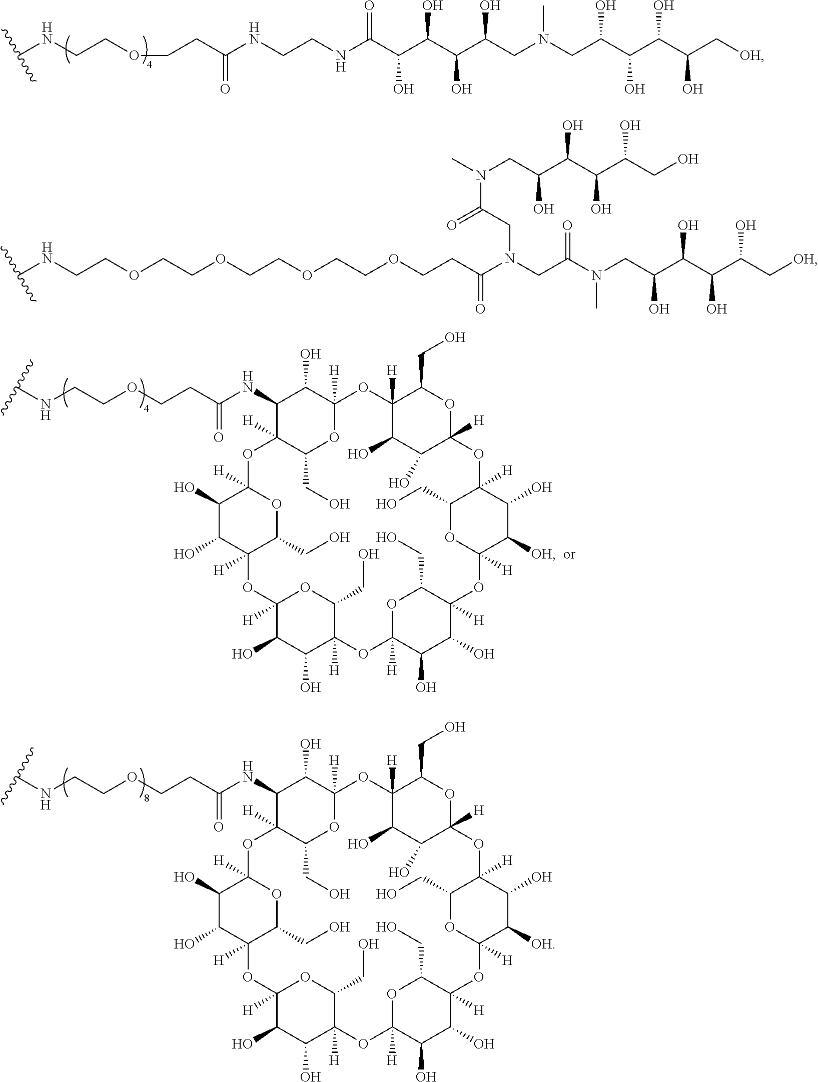

60. The compound of any one of claims 32-59, wherein each of p and q is, independently, 2 or 4.

61. The compound of any one of claims 32-60, wherein Payload is a residue of one of the following formulas:

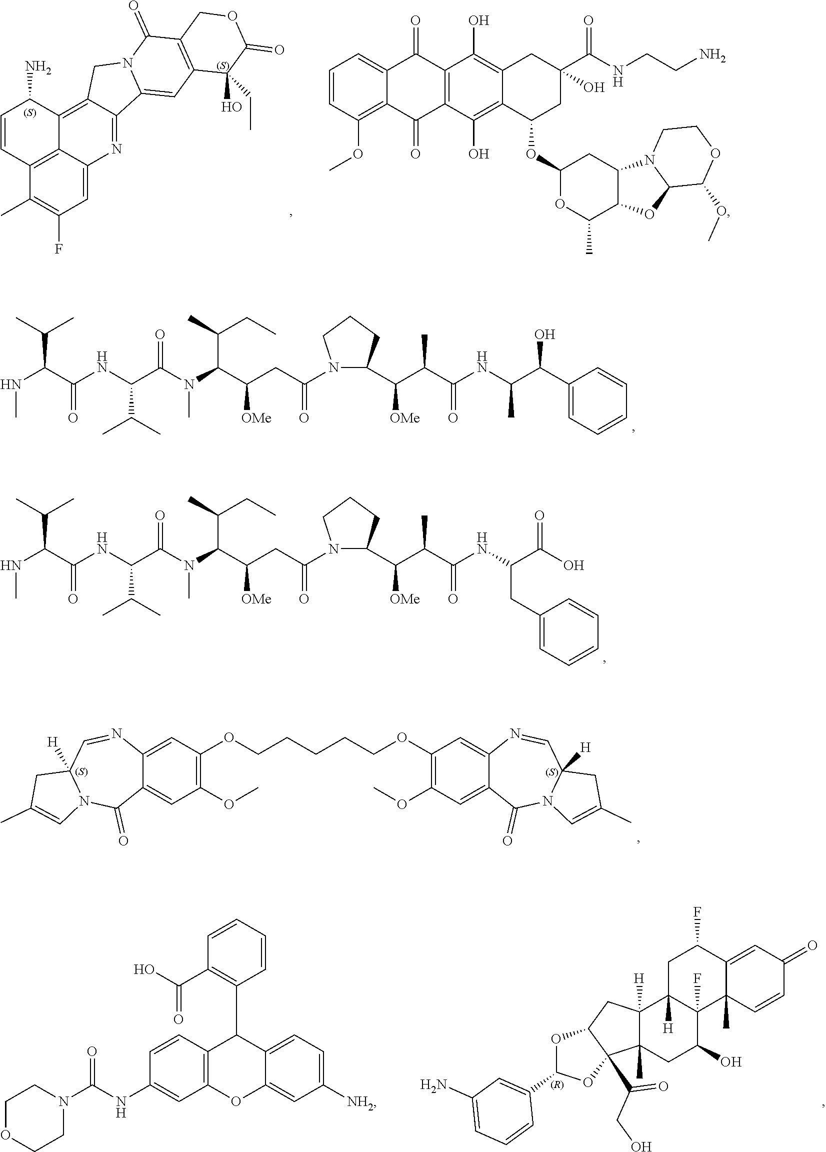

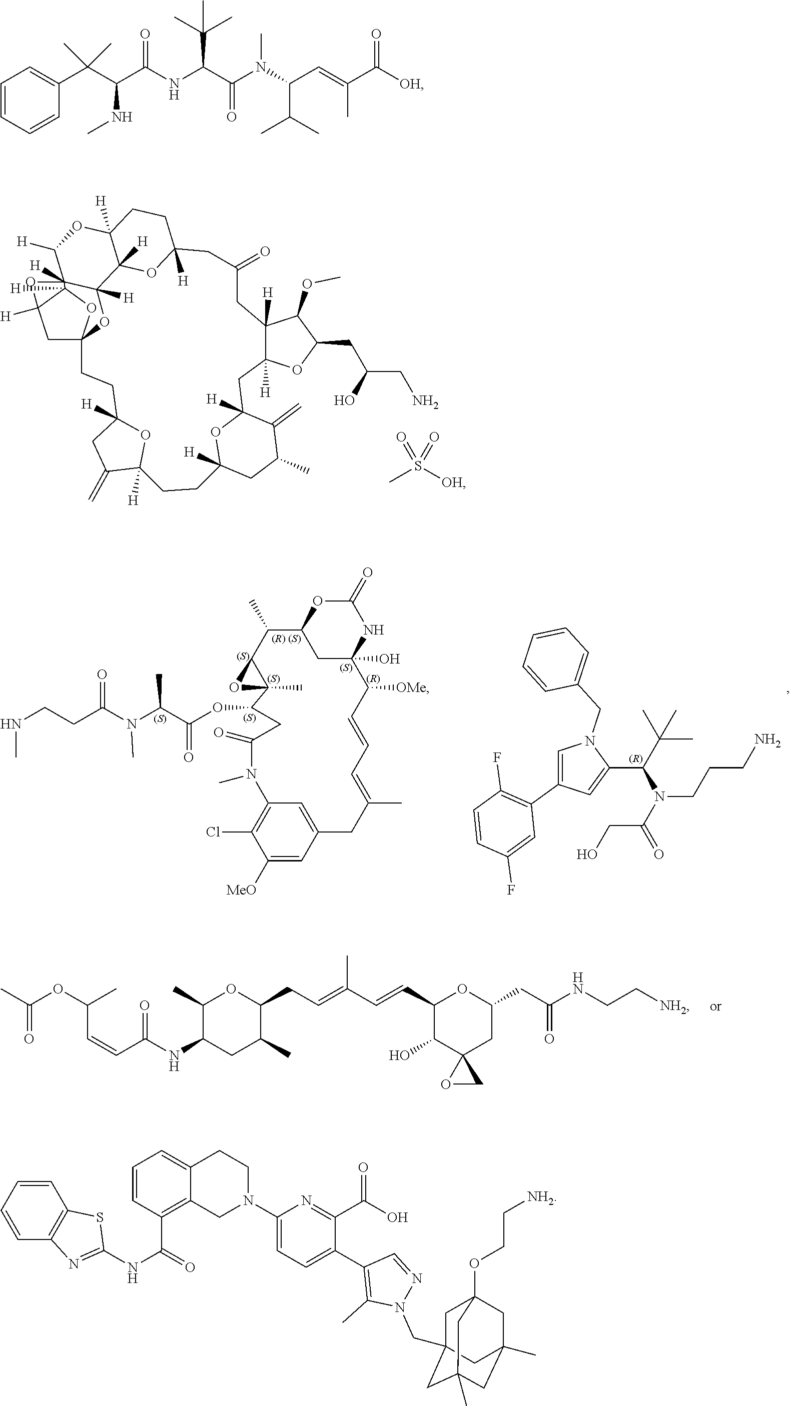

62. The compound of any one of claims 32-61, wherein Payload is

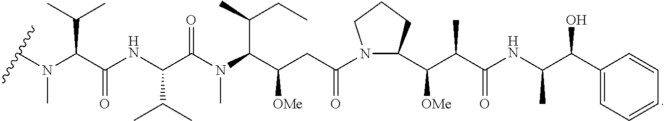 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

63. The compound of any one of claims 32-62, wherein BA binds to one or more receptors selected from B7H3, PTK7, or CLL1.

64. The compound of any one of claims 32-63, wherein BA is ifinatamab, 6E7, or cofetuzumab, or an antigen-binding fragment of ifinatamab, 6E7, or cofetuzumab.

65. The compound of claim 32, wherein the compound is 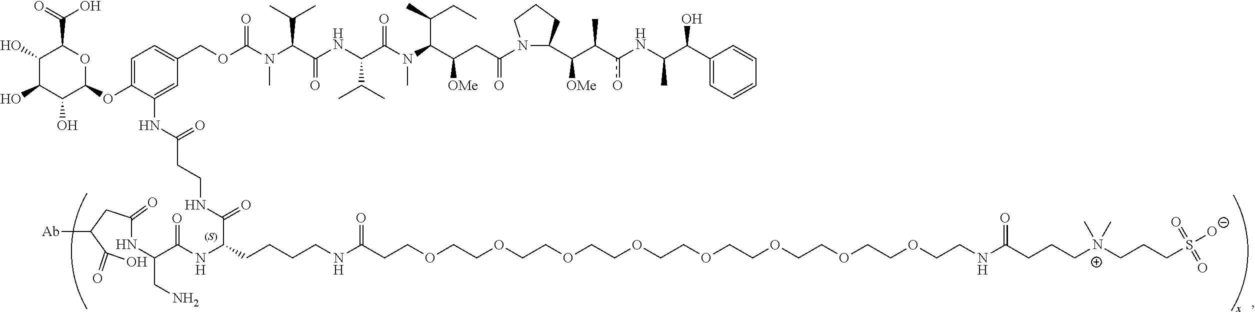
wherein Ab is 6E7 or an antigen-binding fragment thereof; 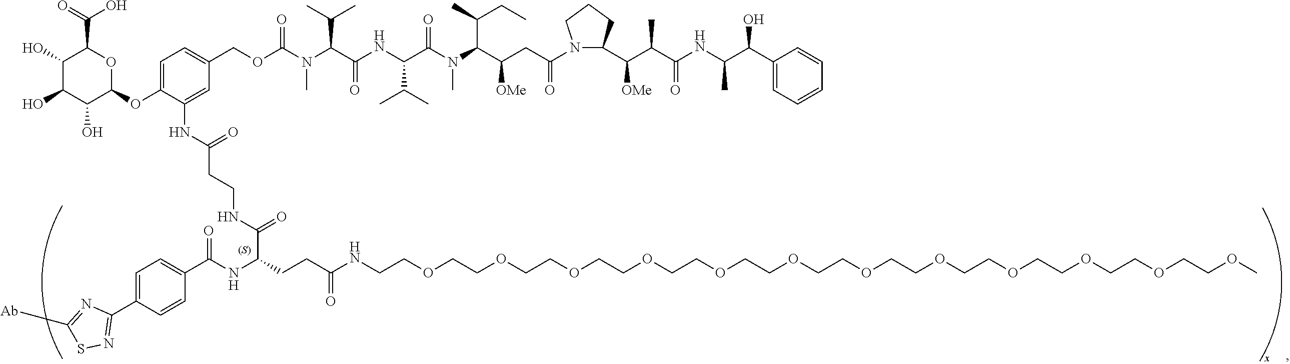
wherein Ab is 6E7 or an antigen-binding fragment thereof; 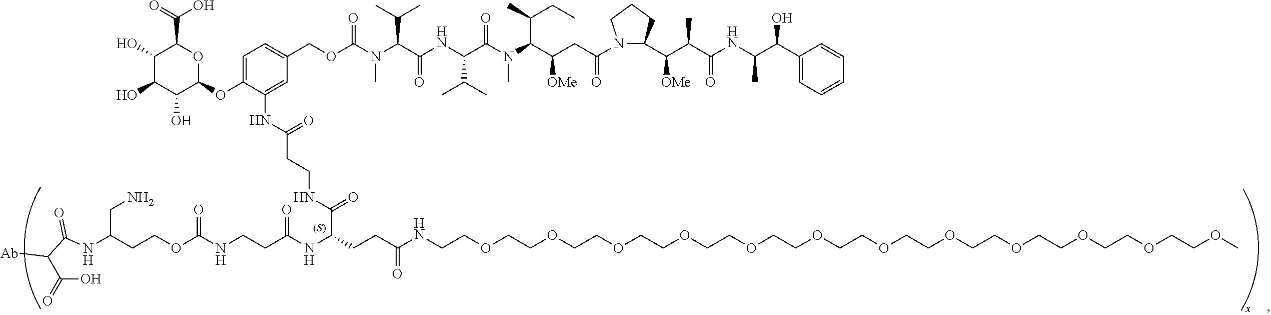
wherein Ab is 6E7 or an antigen-binding fragment thereof; 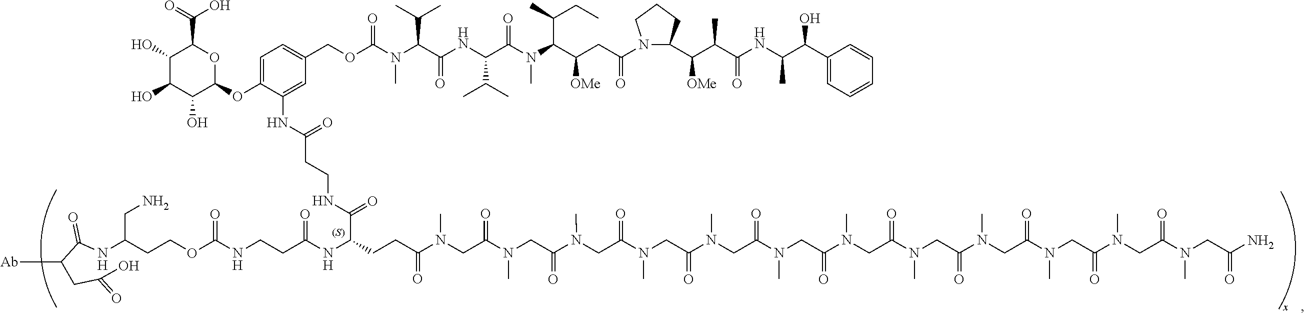
wherein Ab is 6E7 or an antigen-binding fragment thereof; 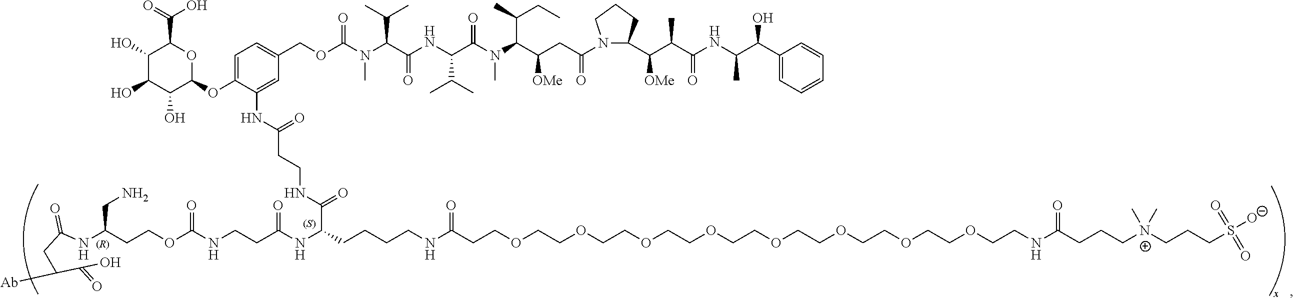
wherein Ab is 6E7 or an antigen-binding fragment thereof; 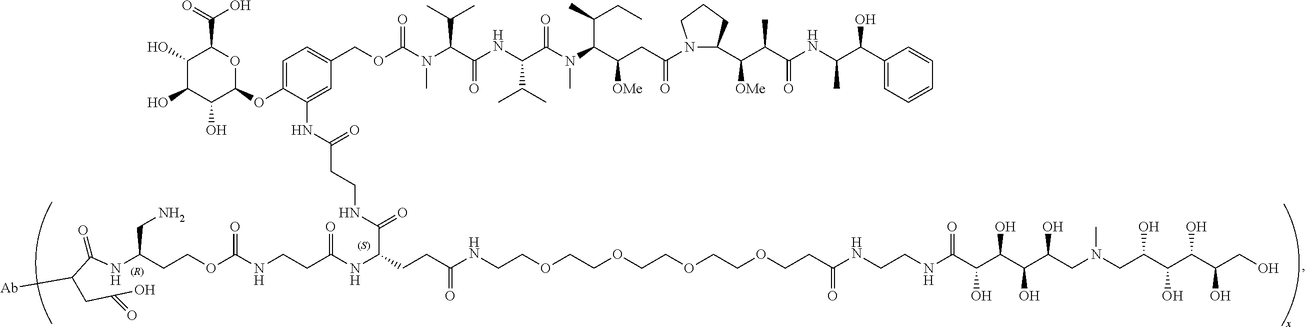
wherein Ab is 6E7 or an antigen-binding fragment thereof; 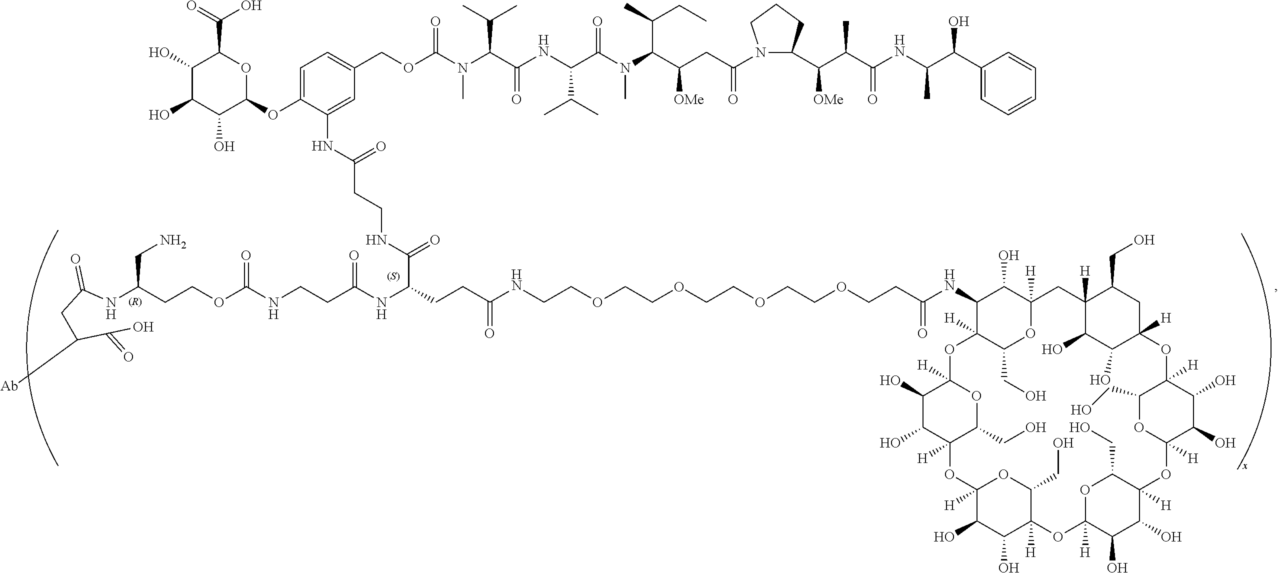
wherein Ab is 6E7 or an antigen-binding fragment thereof; 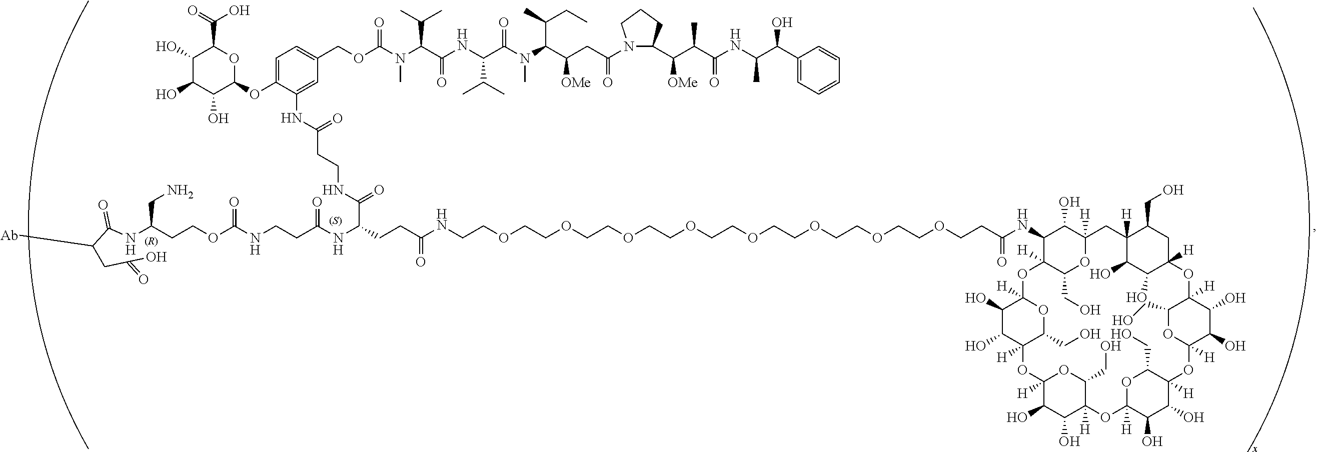
wherein Ab is 6E7 or an antigen-binding fragment thereof; 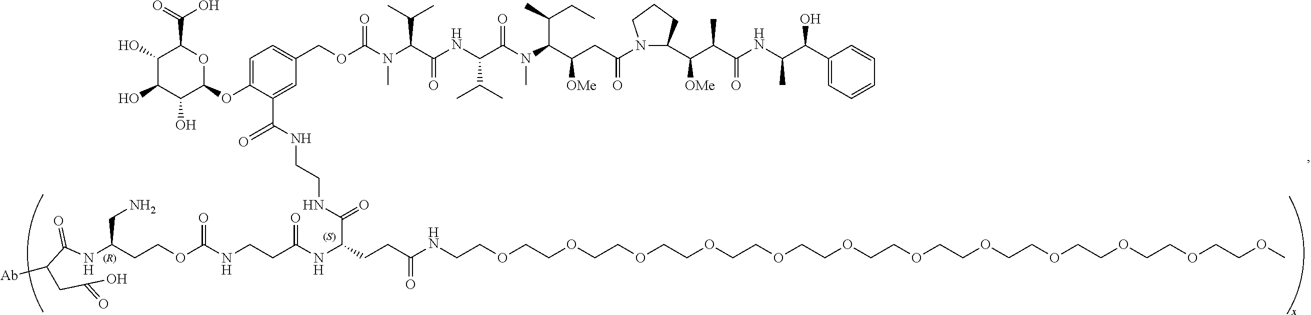
wherein Ab is 6E7 or an antigen-binding fragment thereof; 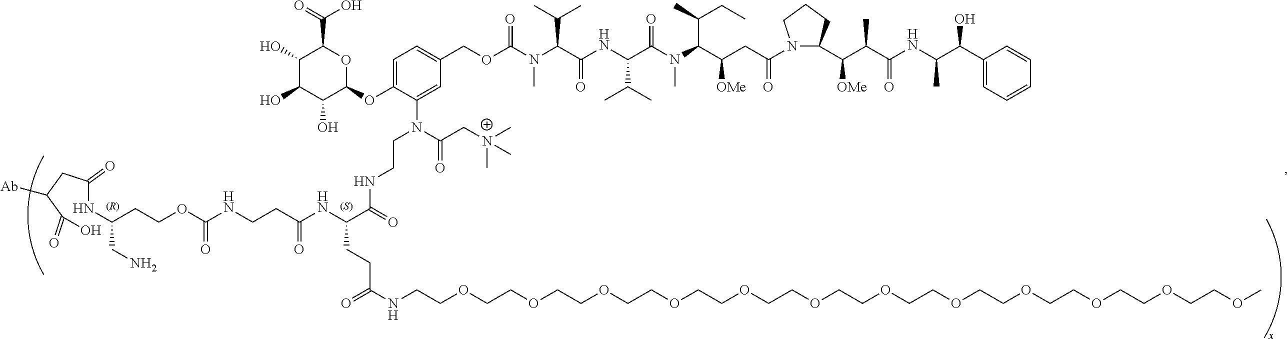
wherein Ab is 6E7 or an antigen-binding fragment thereof; 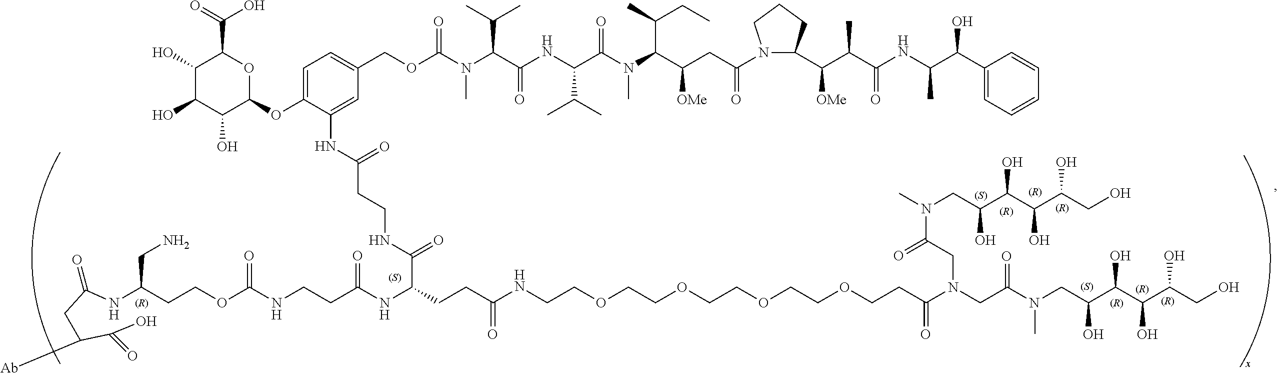
wherein Ab is 6E7 or an antigen-binding fragment thereof; 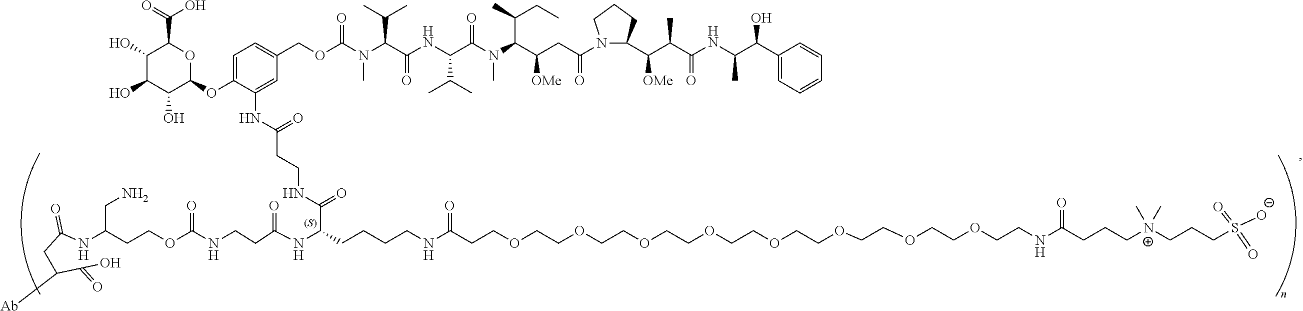
wherein Ab is ifinatamab or an antigen-binding fragment thereof; 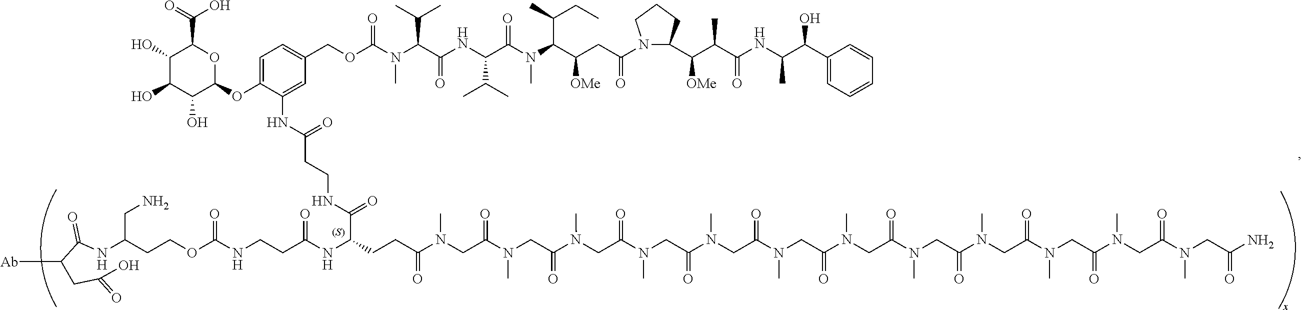
wherein Ab is cofetuzumab or an antigen-binding fragment thereof; or 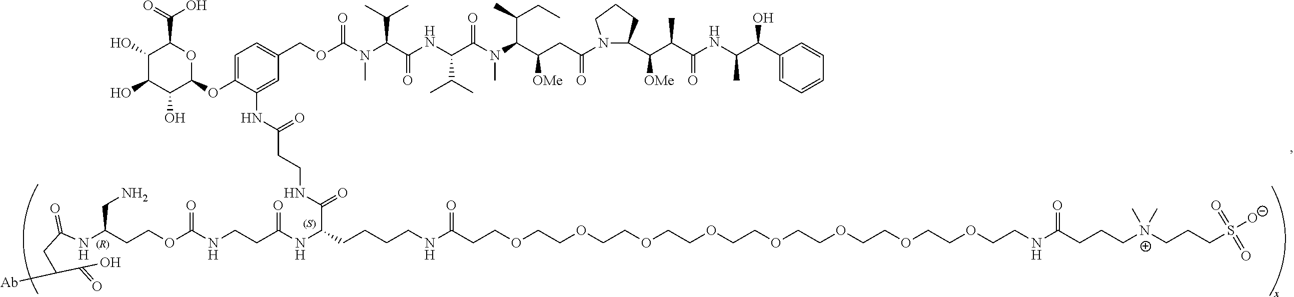
wherein Ab is cofetuzumab or an antigen-binding fragment thereof,
or a pharmaceutically acceptable salt, tautomer, solvate, or stereoisomer of any of the foregoing.

66. The compound of any one of claims 32-65, wherein x is about 3.5 to about 4.5.

67. The compound of claim 66, wherein the compound is 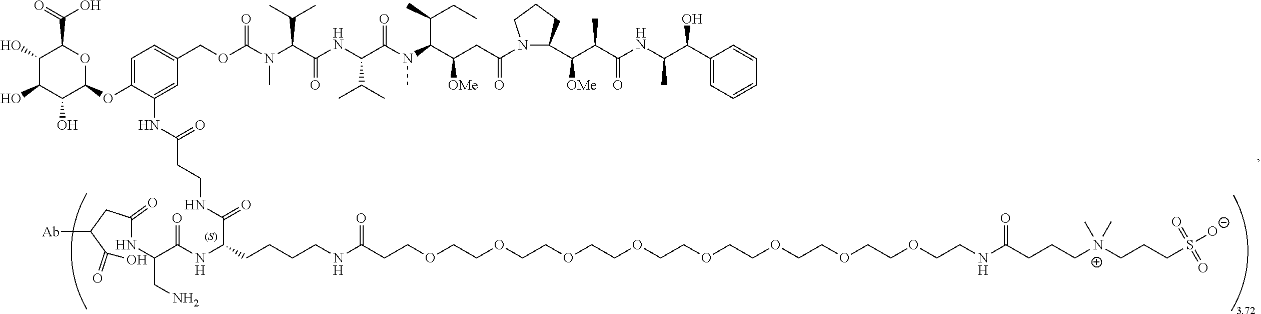
wherein Ab is 6E7 or an antigen-binding fragment thereof; 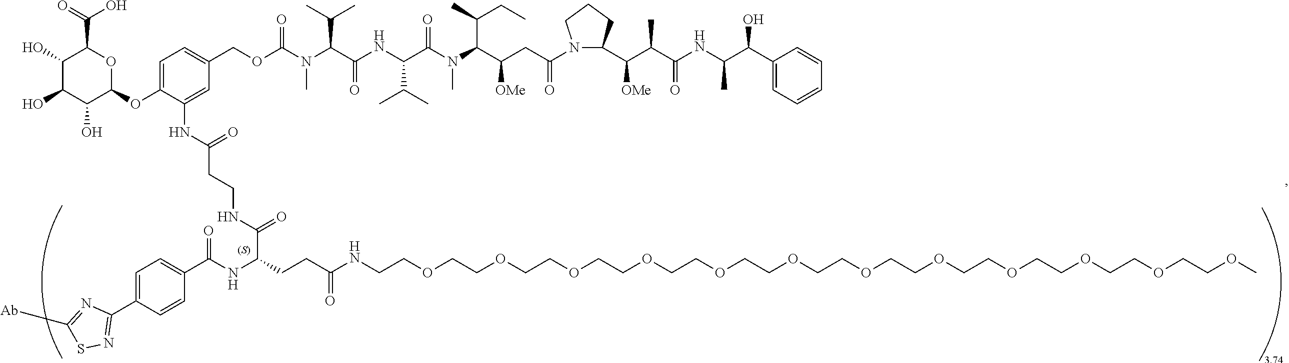
wherein Ab is 6E7 or an antigen-binding fragment thereof; 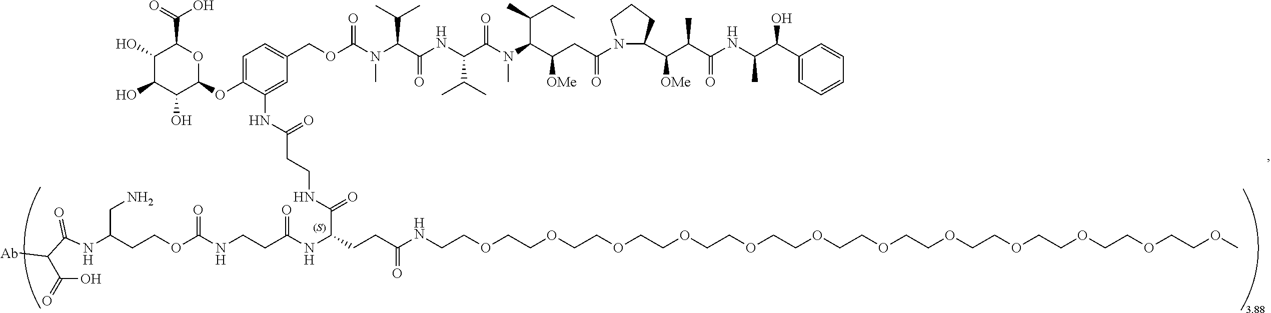
wherein Ab is 6E7 or an antigen-binding fragment thereof; 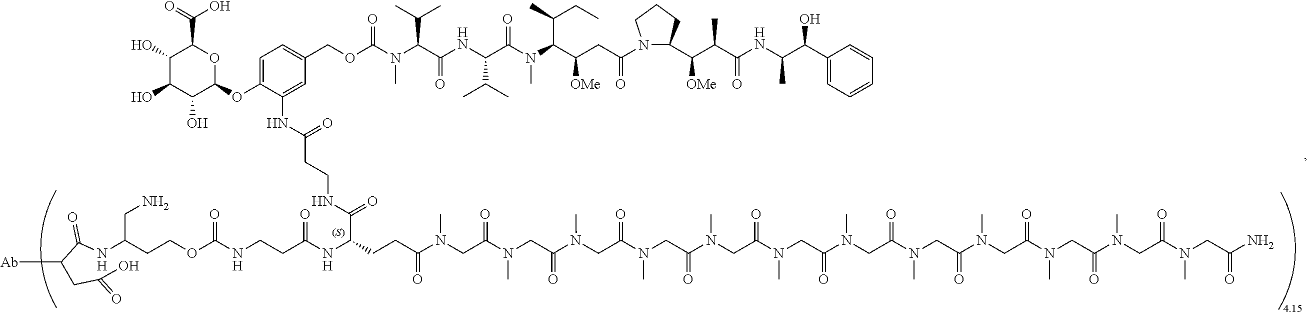
wherein Ab is 6E7 or an antigen-binding fragment thereof; 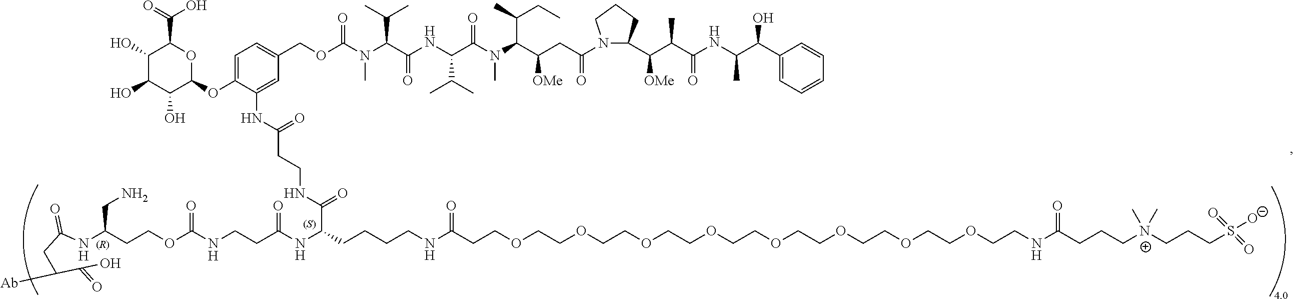
wherein Ab is 6E7 or an antigen-binding fragment thereof; 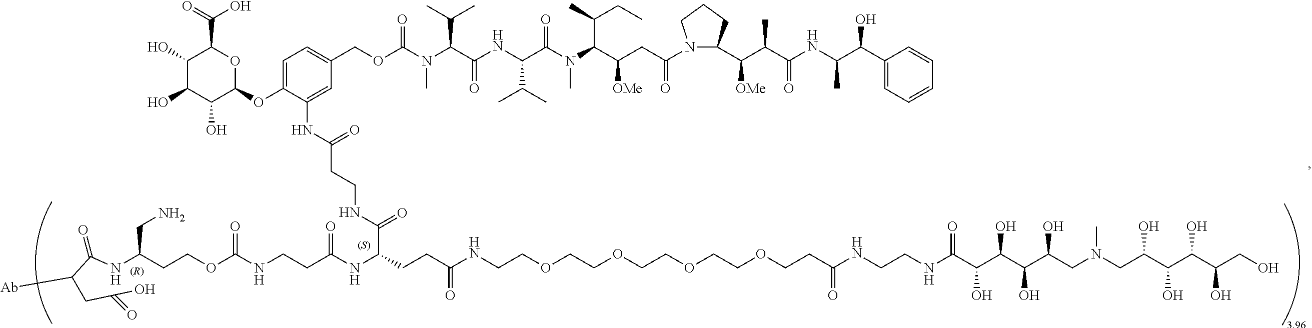
wherein Ab is 6E7 or an antigen-binding fragment thereof; 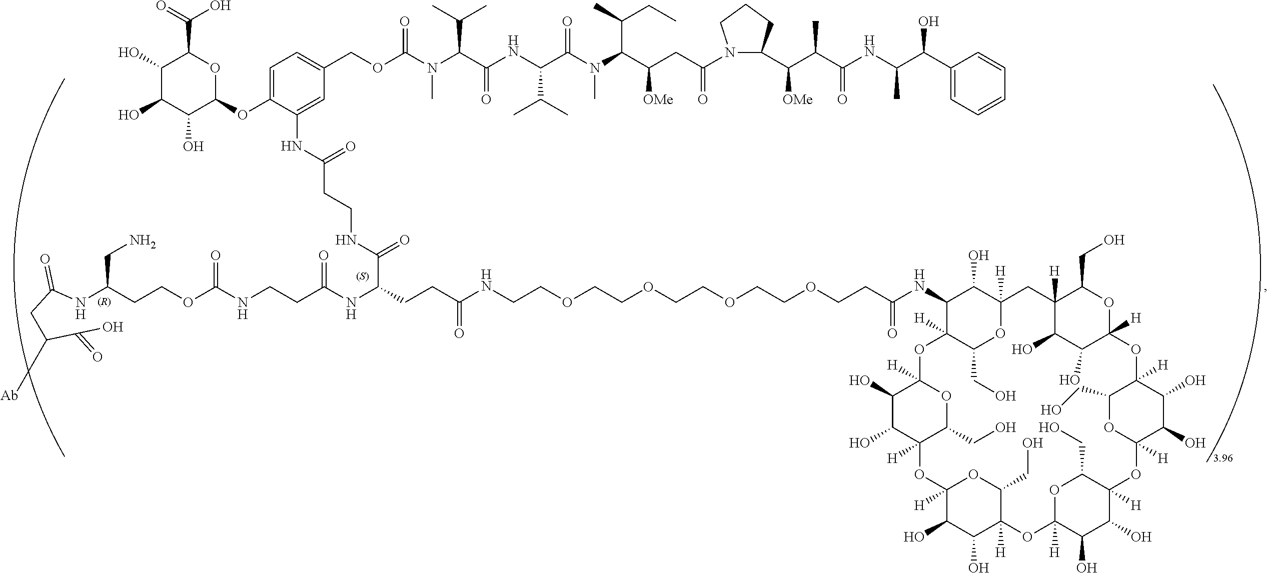
wherein Ab is 6E7 or an antigen-binding fragment thereof; 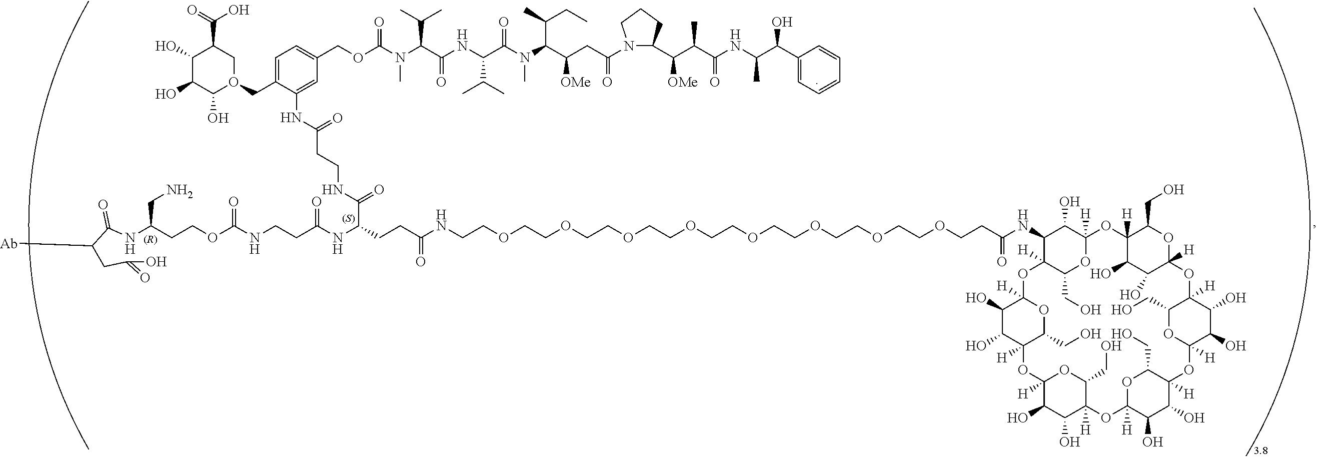
wherein Ab is 6E7 or an antigen-binding fragment thereof; 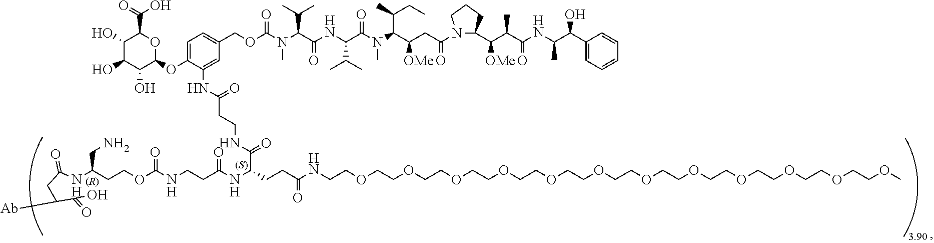
wherein Ab is 6E7 or an antigen-binding fragment thereof; 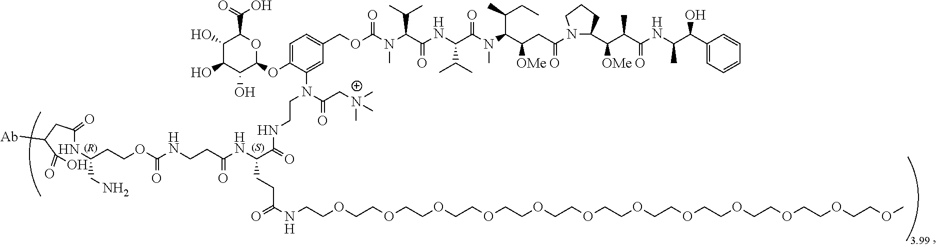
wherein Ab is 6E7 or an antigen-binding fragment thereof; 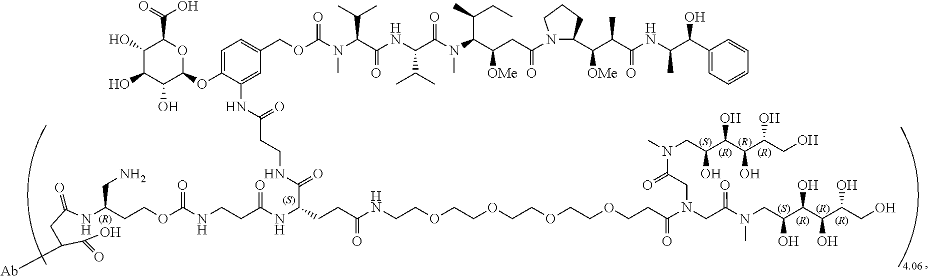
wherein Ab is 6E7 or an antigen-binding fragment thereof; 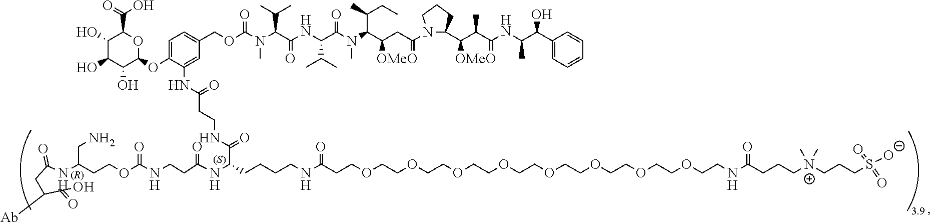
wherein Ab is ifinatamab or an antigen-binding fragment thereof; 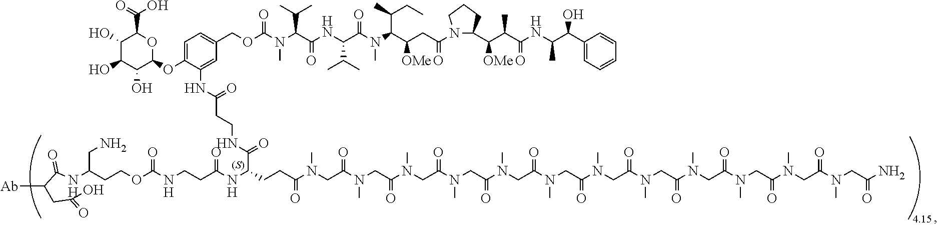
wherein Ab is cofetuzumab or an antigen-binding fragment of thereof; or 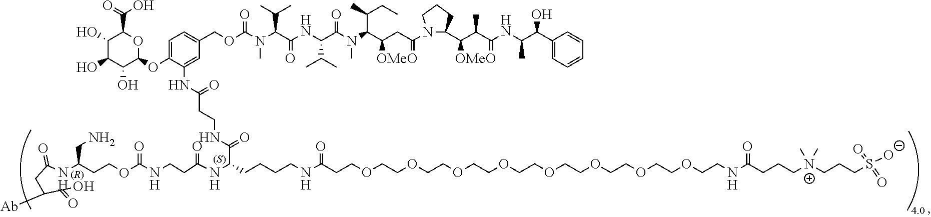
wherein Ab is cofetuzumab or an antigen-binding fragment of thereof,
or a pharmaceutically acceptable salt, tautomer, solvate, or stereoisomer of any of the foregoing.

68. A compound of formula (I-I): 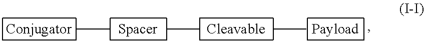
or a pharmaceutically acceptable salt, tautomer, solvate, or stereoisomer thereof, wherein:
Conjugator has formula (I-II) or (I-III): 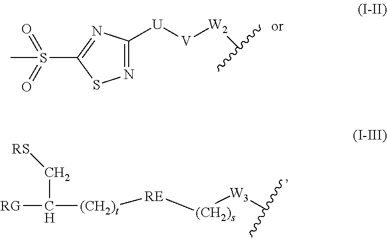
wherein:
U is a bond, heteroarylene, or arylene;
V is a bond or —C≡C—(CH₂)ₙ—;
n is an integer from 0 to 10 inclusive;
W₂ is —C(═O)—, —NH—, or —O—;
RG is 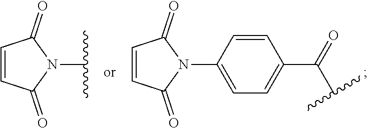 [SMILES: *N1C(=O)C=CC1=O ; *C(=O)c1ccc(N2C(=O)C=CC2=O)cc1]
RS is —NR^{1a}R^{1b};
each of R^{1a} and R^{1b} is, independently, H or substituted or unsubstituted C₁₋₄ alkyl;
RE is a bond, —O—, —OC(═O)—, —OC(═O)NR⁶—, —NHC(═O)NR⁶—, —OS(═O)₂NR⁶—, —NHS(═O)₂NR⁶—, or —OC(═O)NHS(═O)₂NR⁶—;
R⁶ is H or substituted or unsubstituted C₁₋₄ alkyl;
W₃ is —C(═O)—, —NH—, or —O—;
each of s and t is, independently, 0, 1, or 2, and [symbol]
indicates a point of covalent attachment within the compound;
Spacer is a bond, **—NH—(CH₂CH₂O)ₘ—CH₂CH₂—C(═O)—, **—(CH₂)ₘ—C(═O)—, **—CH₂—C(═O)—NH—(CH₂)ₘ—C(═O)—, **—(CH₂CH₂O)ₘ—CH₂CH₂—C(═O)—, **—CH[—(CH₂)ₘ—COOH]—C(═O)—, **—CH₂—C(═O)—NH—(CH₂)ₘ—C(═O)—NH—(CH₂)ₘ—C(═O)—, **—C(═O)—(CH₂)ₘ—C(═O)—, **—NH—(CH₂)ₘ—C(═O)—, or **—NH—(CH₂)ₘ—O—, wherein:
m is an integer from 1 to 12 inclusive, and
** marks the bond where Spacer connects to Conjugator;
Cleavable has formula (IVa), (IVb), (IVc), (Va), (Vb), (VIa), (VIb), (VIIa), or (VIIb): 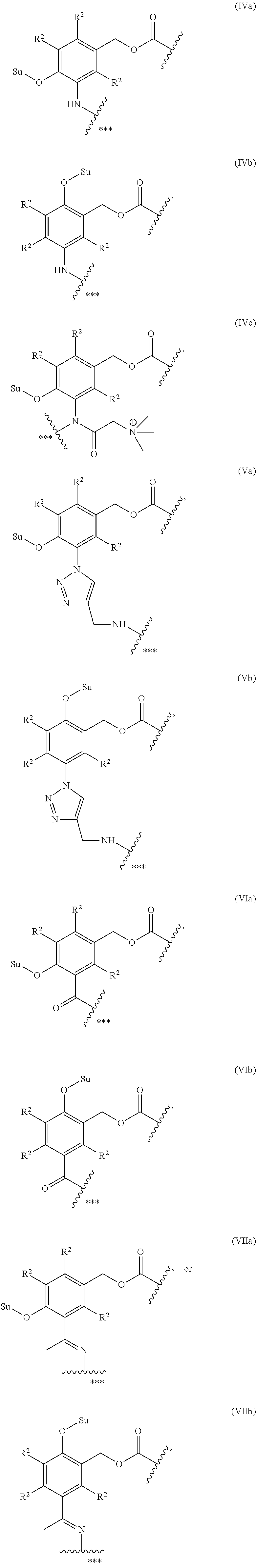
wherein
Su is a Sugar moiety,
each R² is, independently, hydrogen, halogen, substituted or unsubstituted C₁₋₄ alkyl, —CN, or —NO₂, and
*** marks the bond where Cleavable connects to Spacer; and
Payload is a payload residue.

69. The compound of claim 68, wherein Conjugator has formula (I-II).

70. The compound of claim 69, wherein U is arylene.

71. The compound of claim 70, wherein U is phenylene.

72. The compound of claim 71, wherein U is

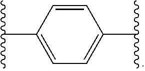 [SMILES: *c1ccc(*)cc1]

73. The compound of claim 69, wherein U is heteroarylene.

74. The compound of claim 73, wherein U is a bivalent pyrimidine ring.

75. The compound of claim 74, wherein U is

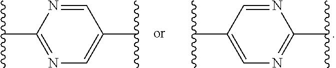 [SMILES: *c1cnc(*)nc1 ; *c1cnc(*)nc1]

76. The compound of claim 69, wherein U is a bond.

77. The compound of any one of claims 68-76, wherein V is a bond.

78. The compound of any one of claims 68-76, wherein V is —C≡C—(CH₂)ₙ—.

79. The compound of claim 78, wherein V is —C≡C—(CH₂)₃—.

80. The compound of any one of claims 68-79, wherein W₂ is —C(═O)—.

81. The compound of claim 68, wherein Conjugator has formula (I-III).

82. The compound of claim 81, wherein RS is —NH₂ or —N(CH₃)₂.

83. The compound of claim 82, wherein RS is —NH₂.

84. The compound of any one of claims 81-83, wherein RE is —OC(═O) NH—.

85. The compound of any one of claims 81-84, wherein RG is

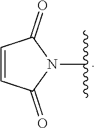 [SMILES: *N1C(=O)C=CC1=O]

86. The compound of any one of claims 81-85, wherein each of s and t is 2.

87. The compound of any one of claims 81-86, wherein W₃ is —C(═O)— or —NH—.

88. The compound of any one of claims 68-87, wherein Spacer is a bond, **—NH—(CH₂CH₂O)ₘ—CH₂CH₂—C(═O)—, or **—NH—(CH₂)ₘ—O—.

89. The compound of any one of claims 68-88, wherein m is 2, 4, 6, or 8.

90. The compound of claim 68, wherein Conjugator has formula (I-IIa1) or (I-IIIa1):

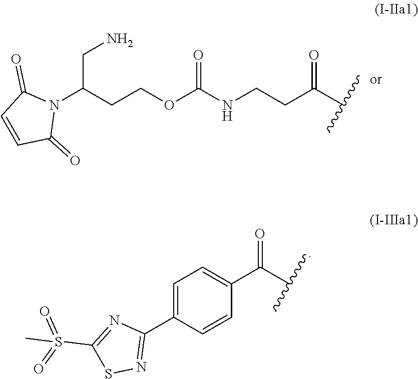 [SMILES: *C(=O)CCNC(=O)OCCC(CN)N1C(=O)C=CC1=O ; *C(=O)c1ccc(-c2nsc(S(C)(=O)=O)n2)cc1]

91. The compound of any one of claims 68-90, wherein Cleavable has formula (IVa1), (IVa2), (IVa3), (Va1), (VIa1), or (VIIa1):

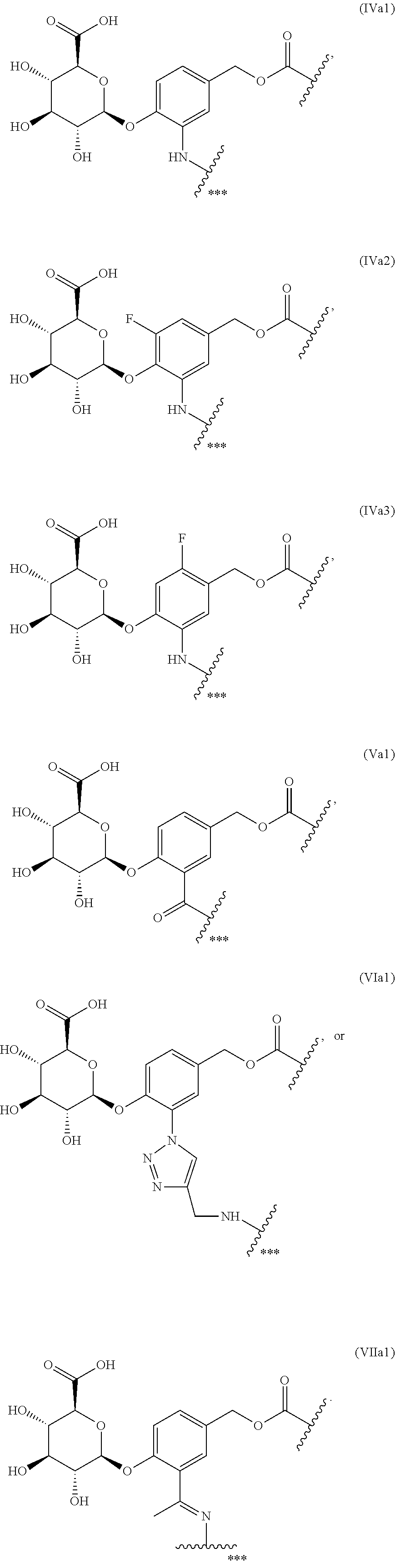 [SMILES: *Nc1cc(COC(*)=O)ccc1O[C@@H]1O[C@H](C(=O)O)[C@@H](O)[C@H](O)[C@H]1O ; *Nc1cc(COC(*)=O)cc(F)c1O[C@@H]1O[C@H](C(=O)O)[C@@H](O)[C@H](O)[C@H]1O ; *Nc1cc(COC(*)=O)c(F)cc1O[C@@H]1O[C@H](C(=O)O)[C@@H](O)[C@H](O)[C@H]1O ; *C(=O)OCc1ccc(O[C@@H]2O[C@H](C(=O)O)[C@@H](O)[C@H](O)[C@H]2O)c(C(*)=O)c1 ; *NCc1cn(-c2cc(COC(*)=O)ccc2O[C@@H]2O[C@H](C(=O)O)[C@@H](O)[C@H](O)[C@H]2O)nn1 ; */N=C(\C)c1cc(COC(*)=O)ccc1O[C@@H]1O[C@H](C(=O)O)[C@@H](O)[C@H](O)[C@H]1O]

92. The compound of any one of claims 68-91, wherein Payload is a residue of one of the following formulas:

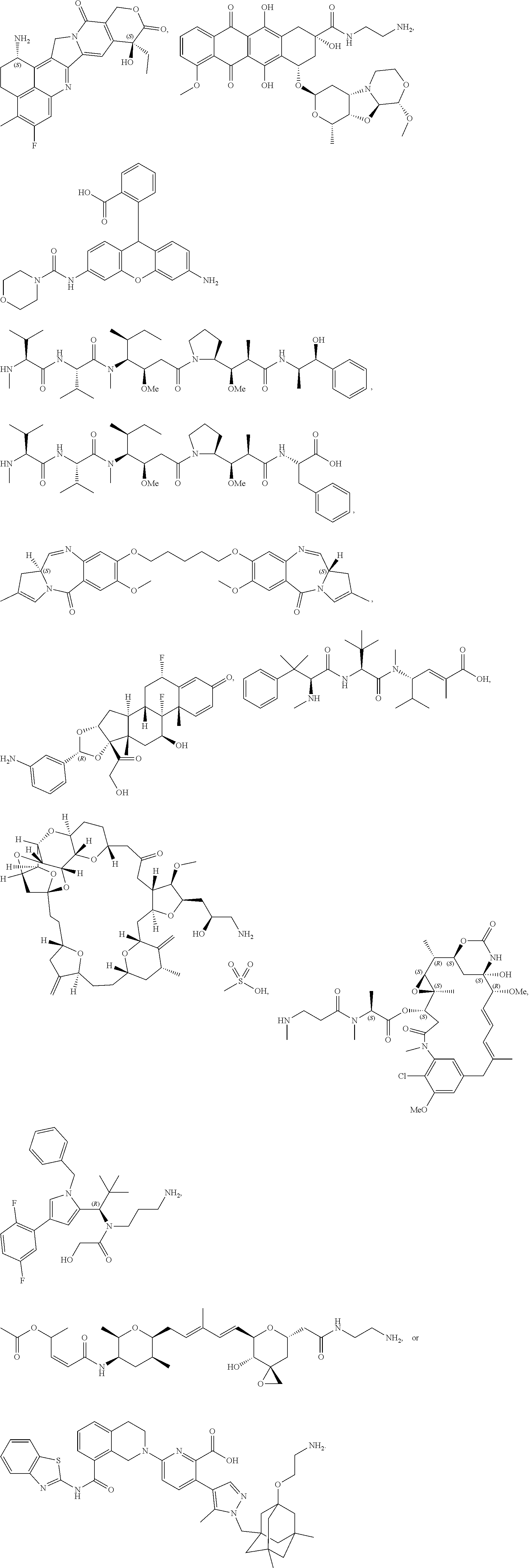

93. The compound of any one of claims 68-92, wherein Payload is

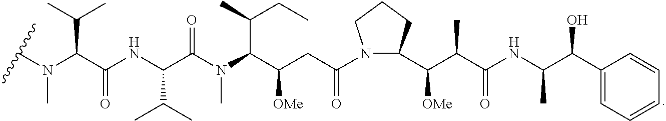 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

94. The compound of claim 68, wherein the compound is 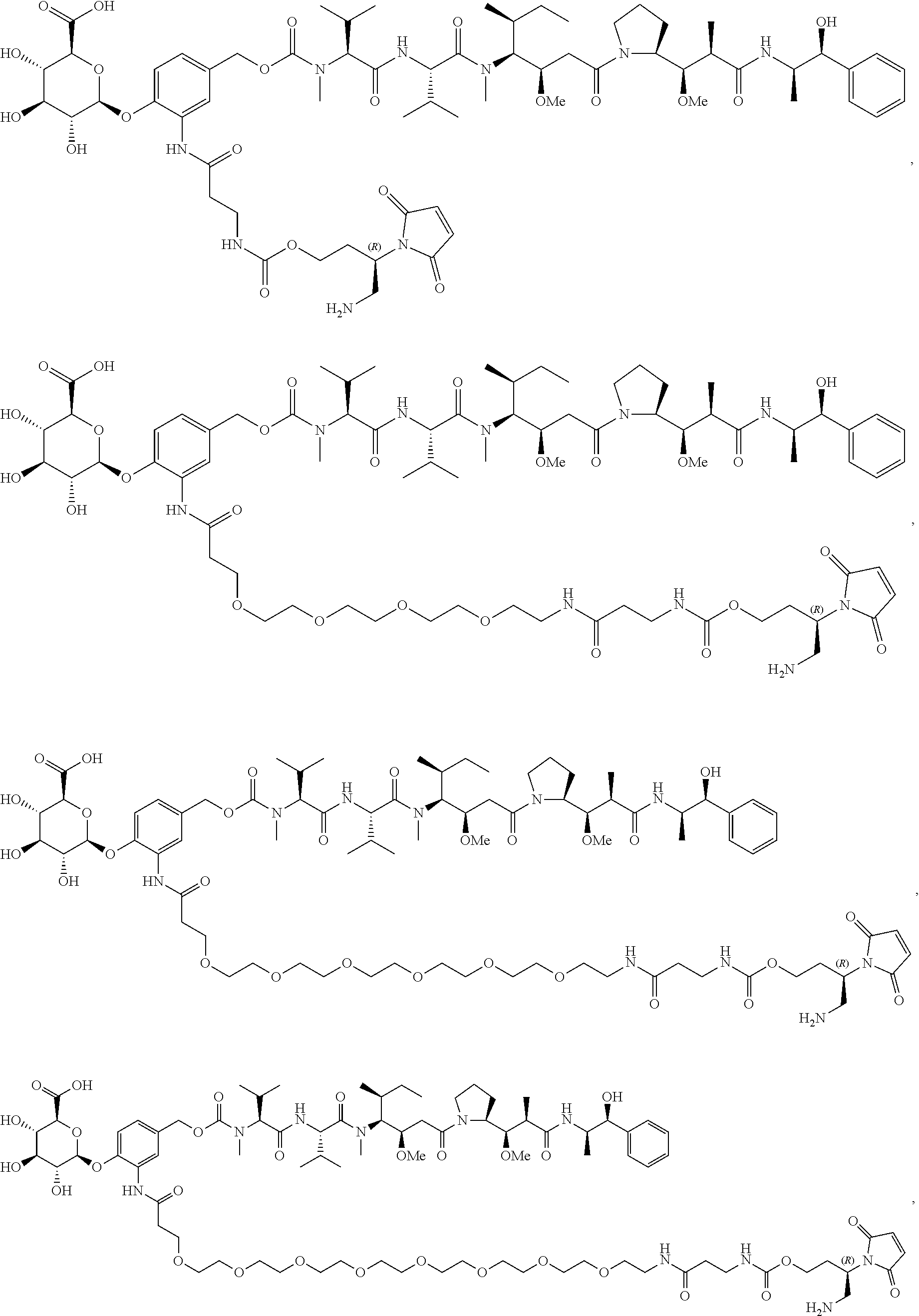 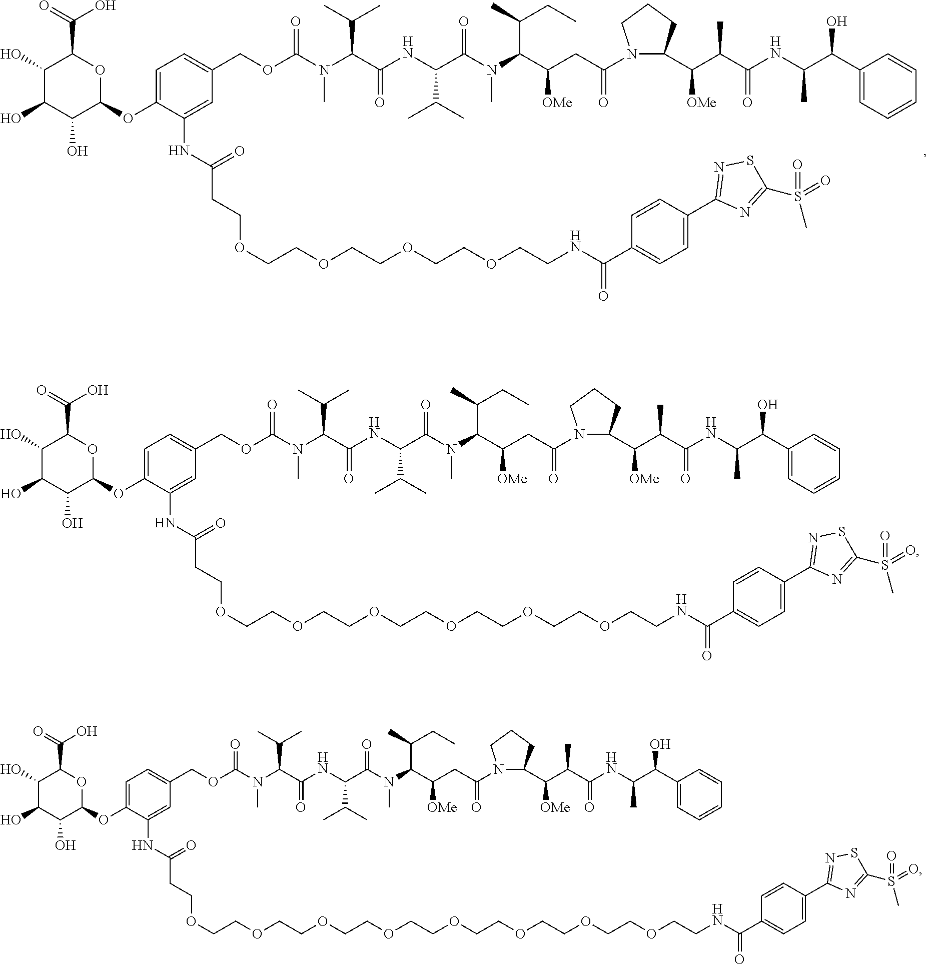 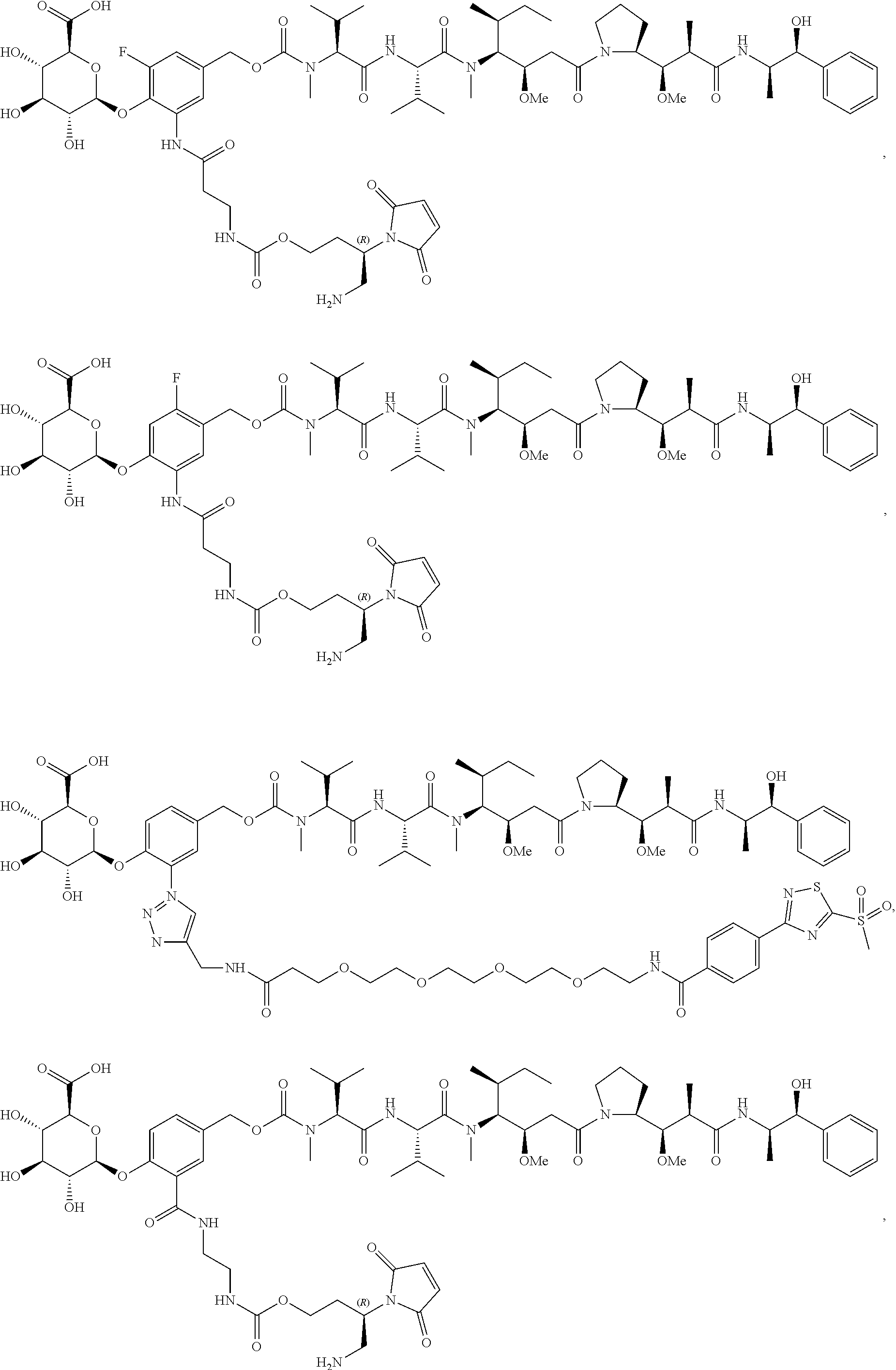 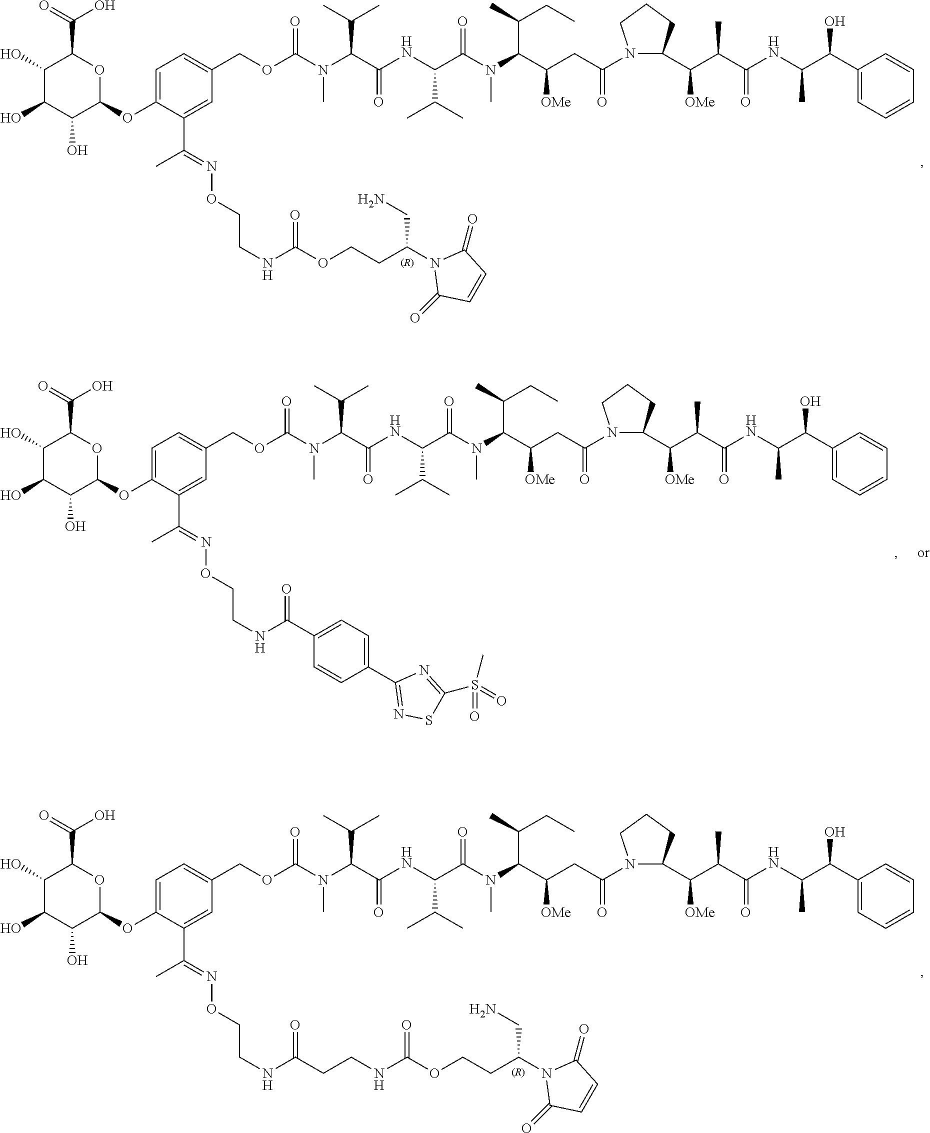
or a pharmaceutically acceptable salt, tautomer, solvate, or stereoisomer thereof.

95. A compound of formula (XV): 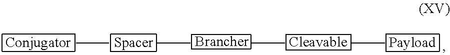
or a pharmaceutically acceptable salt, tautomer, solvate, or stereoisomer thereof, wherein:
Conjugator has formula (I-II) or (I-III): 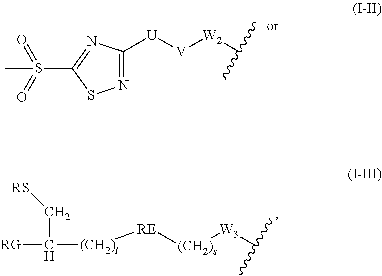
wherein:
U is a bond, heteroarylene, or arylene;
V is a bond or —C≡C—(CH₂)ₙ—;
n is an integer from 0 to 10 inclusive;
W₂ is —C(═O)—, —NH—, or —O—;
RG is 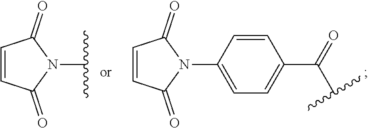 [SMILES: *N1C(=O)C=CC1=O ; *C(=O)c1ccc(N2C(=O)C=CC2=O)cc1]
RS is —NR¹ªR^{1b};
each of R^{1a} and R^{1b} is, independently, H or substituted or unsubstituted C₁₋₄ alkyl;
RE is a bond, —O—, —OC(═O)—, —OC(═O)NR⁶—, —NHC(═O)NR⁶—, —OS(═O)₂NR⁶—, —NHS(═O)₂NR⁶—, or —OC(═O)NHS(═O)₂NR⁶—;
R⁶ is H or substituted or unsubstituted C₁₋₄ alkyl;
W₃ is —C(═O)—, —NH—, or —O—;
each of s and t is, independently, 0, 1, or 2, and [symbol]
indicates a point of covalent attachment within the compound;
Spacer is a bond, **—NH—(CH₂CH₂O)ₘ—CH₂CH₂—C(═O)—, **—(CH₂)ₘ—C(═O)—, **—CH₂—C(═O)—NH—(CH₂)ₘ—C(═O)—, **—(CH₂CH₂O)ₘ—CH₂CH₂—C(═O)—, **—CH[—(CH₂)ₘ—COOH]—C(═O)—, **—CH₂—C(═O)—NH—(CH₂)ₘ—C(═O)—NH—(CH₂)ₘ—C(═O)—, *—C(═O)—(CH₂)ₘ—C(═O)—, **—NH—(CH₂)ₘ—C(═O)—, or **—NH—(CH₂)ₘ—O—, wherein
m is an integer from 1 to 12 inclusive, and
** marks the bond where Spacer connects to Conjugator;
Cleavable has formula (IVa′), (IVb′), (IVc′), (Va′), (Vb′), (VIa′), (VIb′), (VIIa′), or (VIIb′): 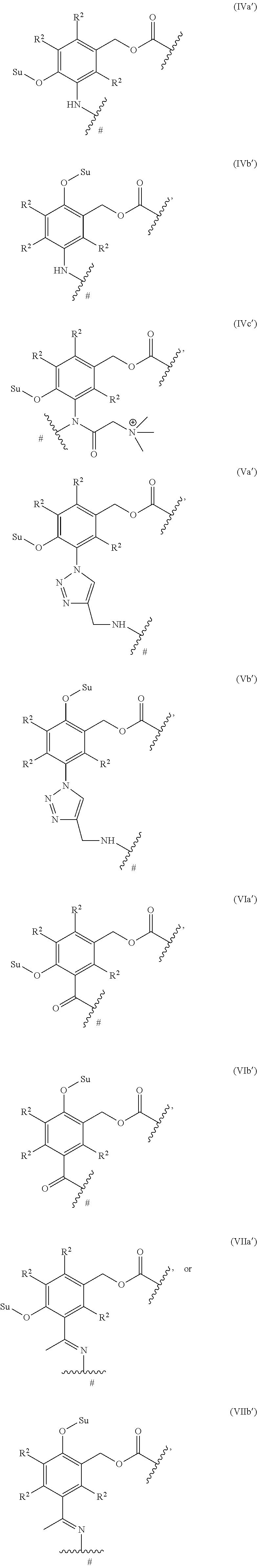
wherein:
Su is a Sugar moiety,
each R² is, independently, hydrogen, halogen, substituted or unsubstituted C₁₋₄ alkyl, —CN, or —NO₂, and
#marks the bond where Cleavable connects to Brancher,
Brancher is selected from formula (XIIa), (XIIb), (XIIc), and (XIId): 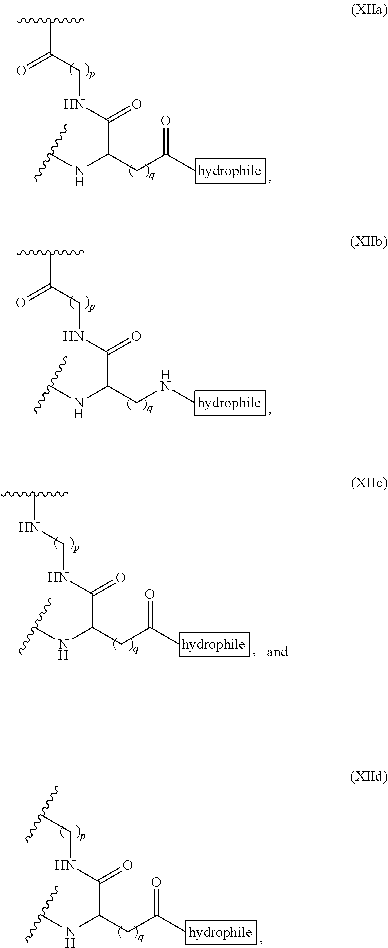
wherein:
each of p and q is, independently, 1, 2, 3, or 4;
hydrophile is —NH—(CH₂CH₂O)ₐ—(CH₂)_{b}CH₃, —N(CH₃)—((CH₂)_{c}C(═O) N(CH₃))ₐ—(CH₂)_{c} C(═O) NH₂, —C(═O)—(CH₂CH₂O)ₐ—(CH₂)_{b}CH₃, —C(═O)—(CH₂CH₂O)ₐ—(CH₂)_{b}NHC(═O)(CH₂)₃N(CH₃)₂ (CH₂)₃S(═O)₂OH, —NH—(CH₂CH₂O)ₐ—(CH₂)_{b}NHC(═O)(CH₂)₃N(CH₃)₂ (CH₂)₃S(═O)₂OH, 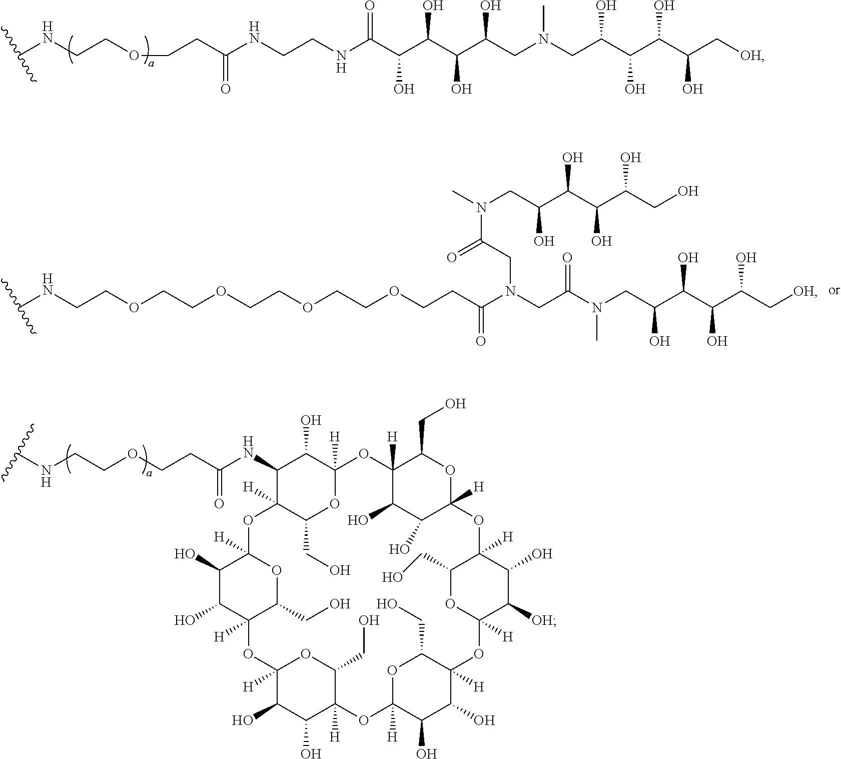
a is an integer from 1 to 18 inclusive;
b is 0, 1, or 2, and
c is 1, 2, 3, or 4; and
Payload is a payload residue.

96. The compound of claim 95, wherein Conjugator has formula (1-11).

97. The compound of claim 96, wherein U is arylene.

98. The compound of claim 97, wherein U is phenylene.

99. The compound of claim 98, wherein U is

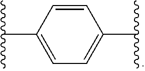 [SMILES: *c1ccc(*)cc1]

100. The compound of claim 96, wherein U is heteroarylene.

101. The compound of claim 100, wherein U is a bivalent pyrimidine ring.

102. The compound of claim 101, wherein U is

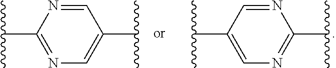 [SMILES: *c1cnc(*)nc1 ; *c1cnc(*)nc1]

103. The compound of claim 96, wherein U is a bond.

104. The compound of any one of claims 95-103, wherein V is a bond.

105. The compound of any one of claims 95-103, wherein V is —C≡C—(CH₂)ₙ—.

106. The compound of claim 105, wherein V is —C≡C—(CH₂)₃—.

107. The compound of any one of claims 95-106, wherein W₂ is —C(═O)—.

108. The compound of claim 95, wherein Conjugator has formula (I-III).

109. The compound of claim 108, wherein RS is —NH₂ or —N(CH₃)₂.

110. The compound of claim 109, wherein RS is —NH₂.

111. The compound of any one of claims 108-110, wherein RE is —OC(═O) NH—.

112. The compound of any one of claims 108-111, wherein RG is

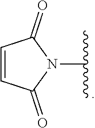 [SMILES: *N1C(=O)C=CC1=O]

113. The compound of any one of claims 108-112, wherein each of s and t is 2.

114. The compound of any one of claims 95-113, wherein Spacer is a bond, **—NH—(CH₂CH₂O)ₘ—CH₂CH₂—C(═O)—, or **—NH—(CH₂)ₘ—O—.

115. The compound of any one of claims 95 and 97-114, wherein m is 2, 4, 6, or 8.

116. The compound of claim 95, wherein Conjugator has formula (I-IIa1) or (I-IIIa1):

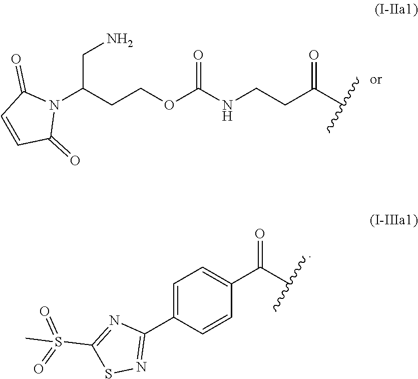 [SMILES: *C(=O)CCNC(=O)OCCC(CN)N1C(=O)C=CC1=O ; *C(=O)c1ccc(-c2nsc(S(C)(=O)=O)n2)cc1]

117. The compound of any one of claims 95-116, wherein Cleavable has formula (IVa′1), (IVc′1), or (Va′1):

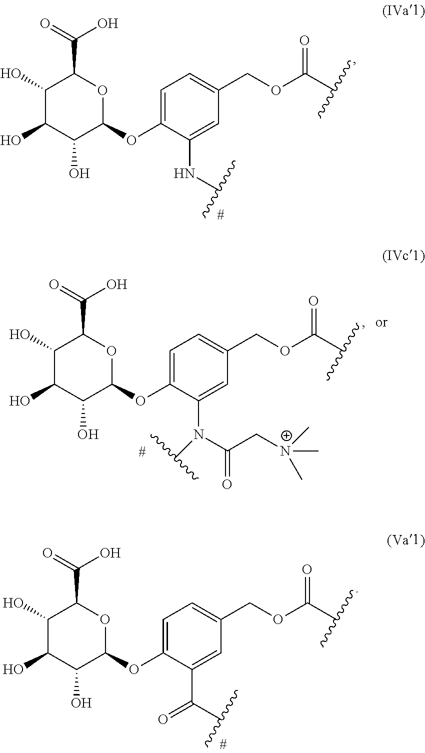 [SMILES: *Nc1cc(COC(*)=O)ccc1O[C@@H]1O[C@H](C(=O)O)[C@@H](O)[C@H](O)[C@H]1O ; *C(=O)OCc1ccc(O[C@@H]2O[C@H](C(=O)O)[C@@H](O)[C@H](O)[C@H]2O)c(N(*)C(=O)C[N+](C)(C)C)c1 ; *C(=O)OCc1ccc(O[C@@H]2O[C@H](C(=O)O)[C@@H](O)[C@H](O)[C@H]2O)c(C(*)=O)c1]

118. The compound of any one of claims 95-117, wherein Payload is a residue of one of the following formulas:

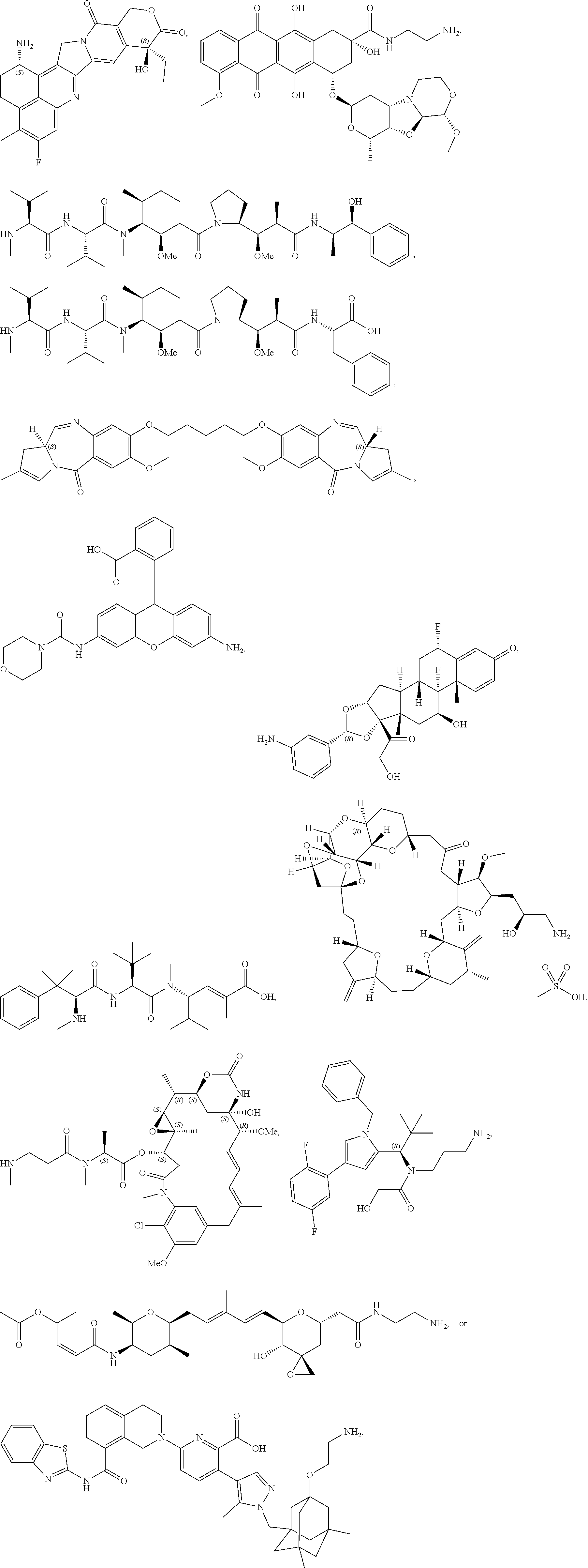

119. The compound of any one of claims 95-118, wherein Payload is

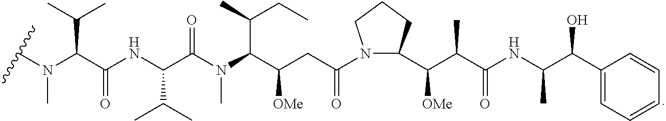 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

120. The compound of claim 95, the compound is 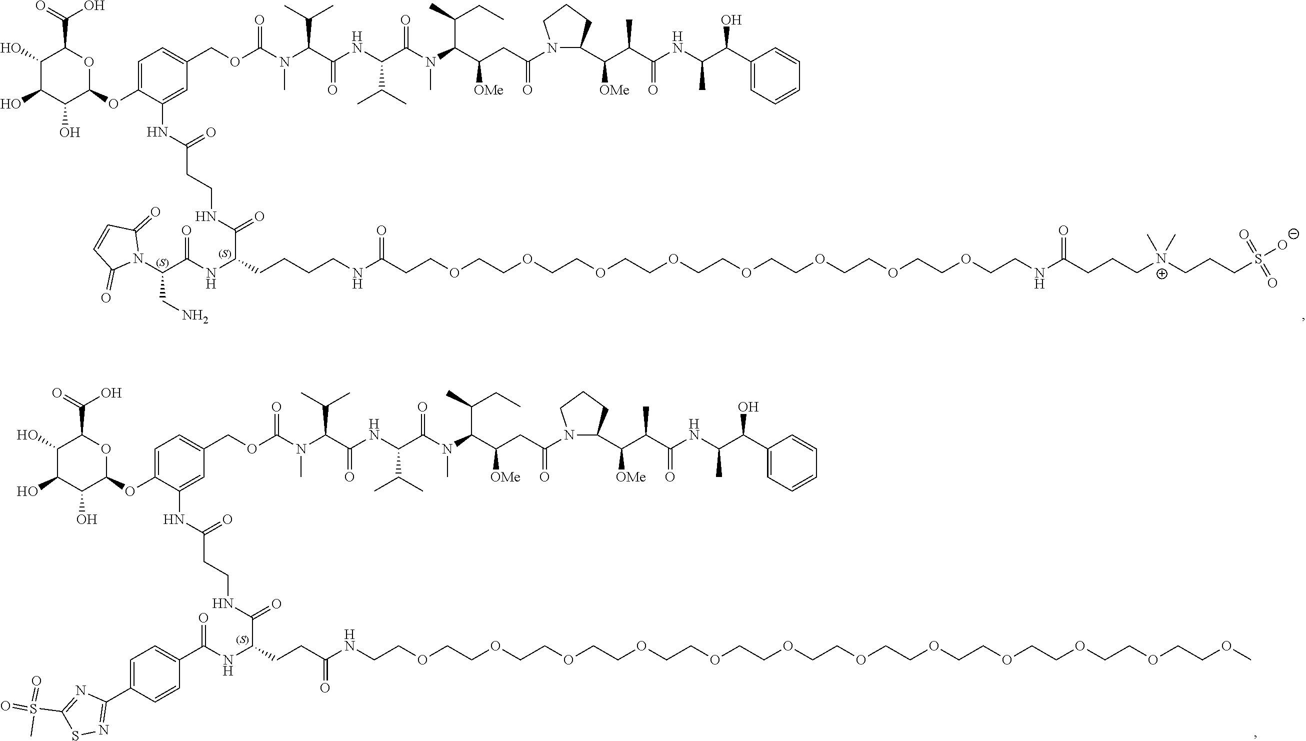 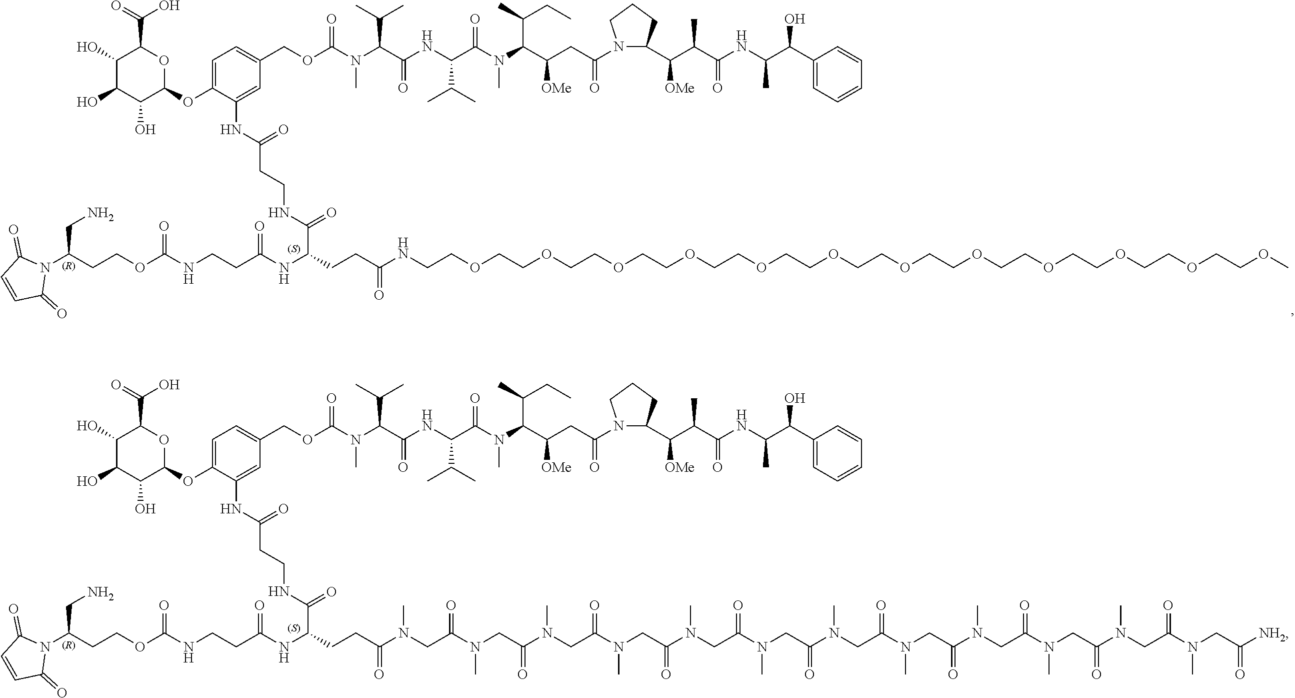 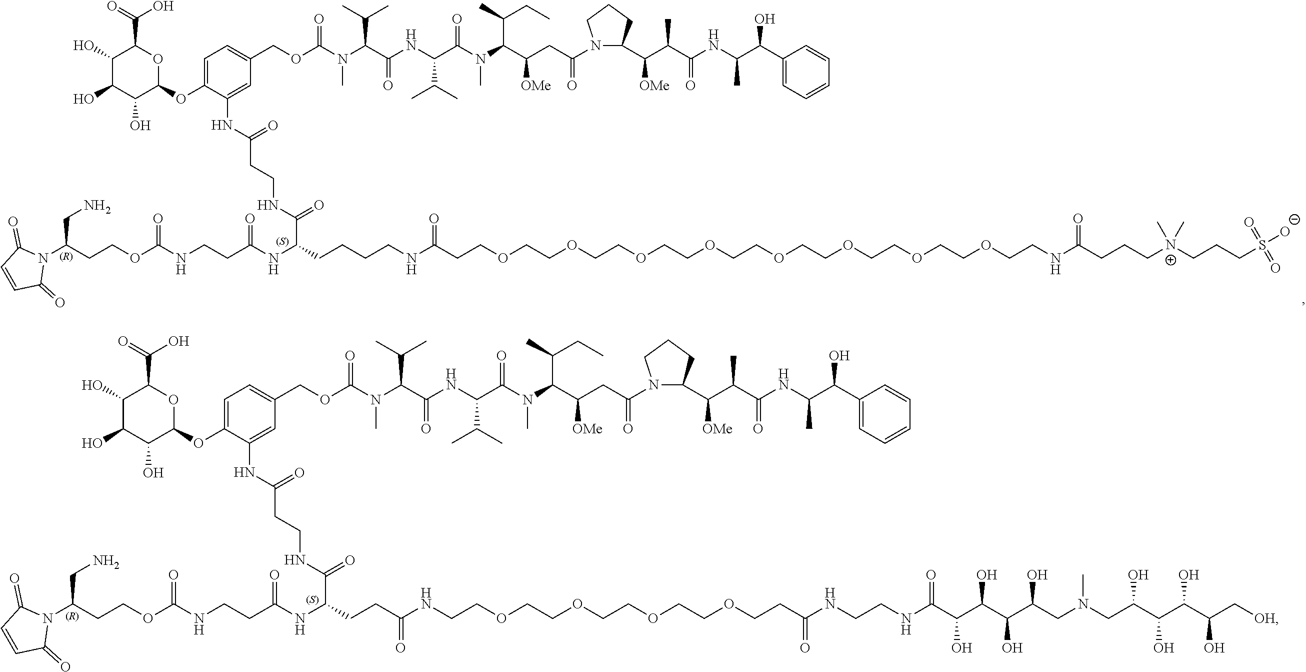 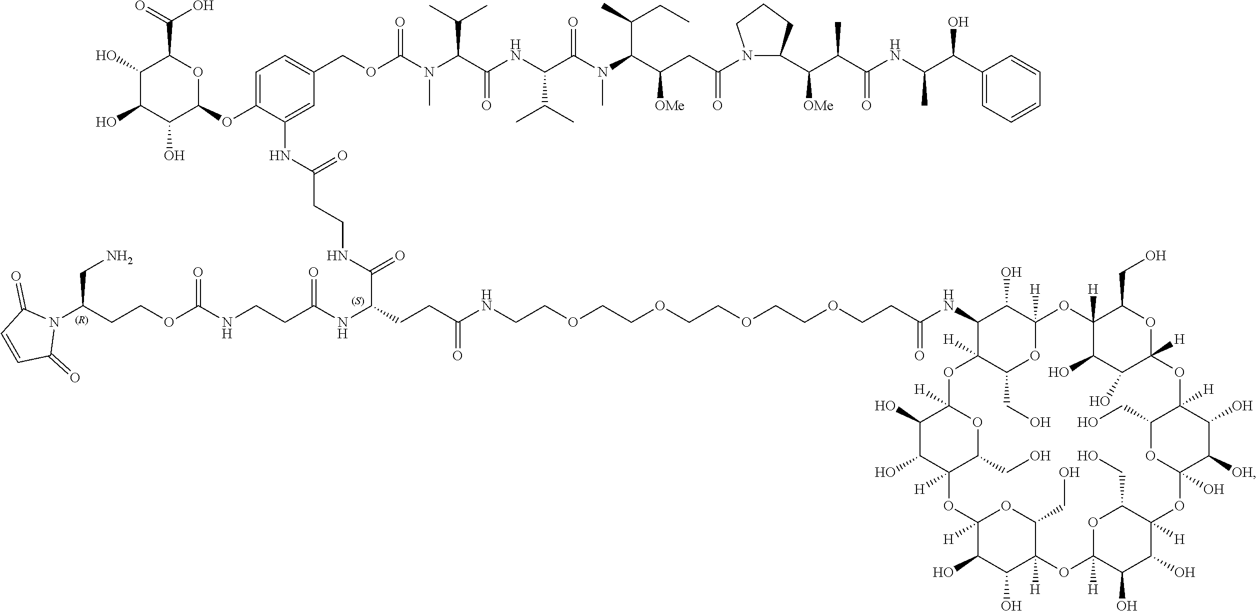 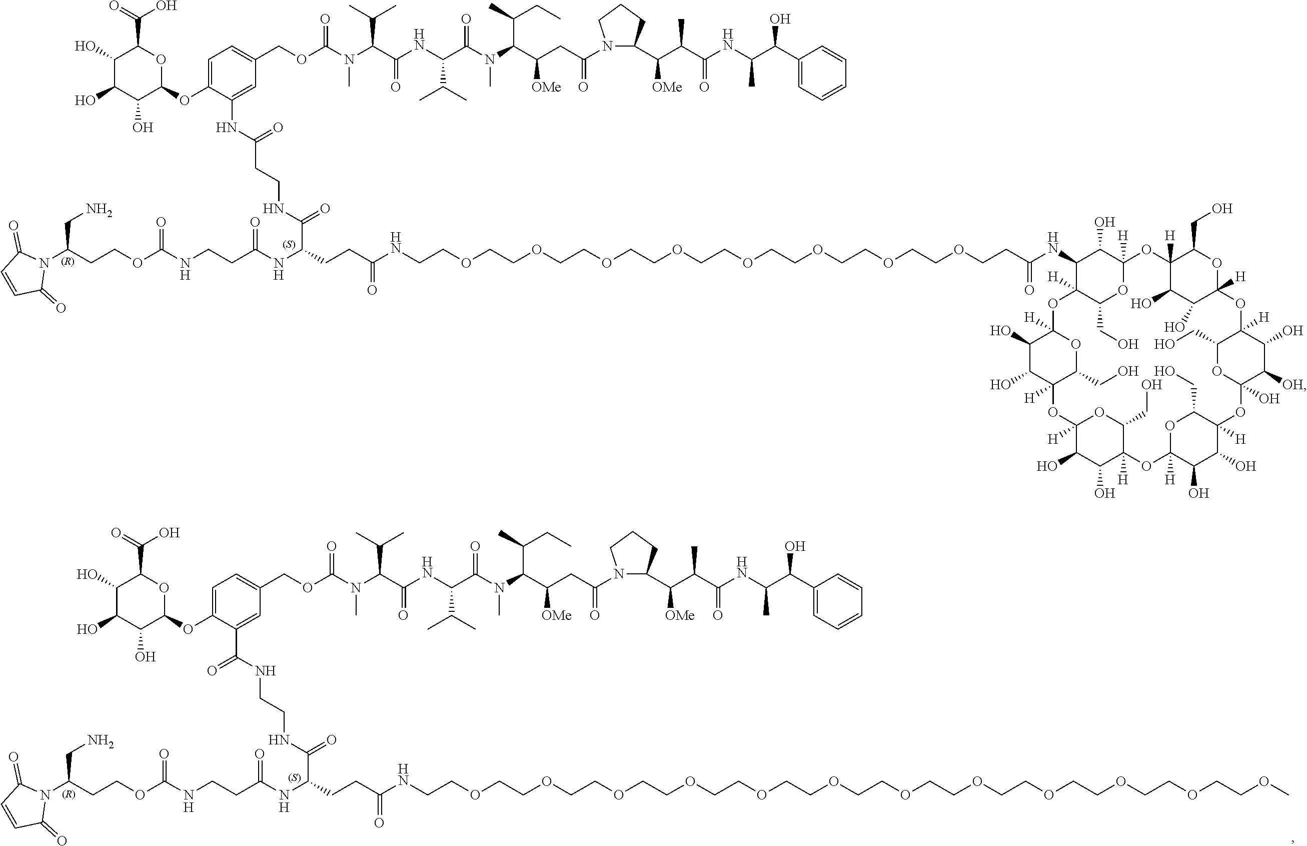 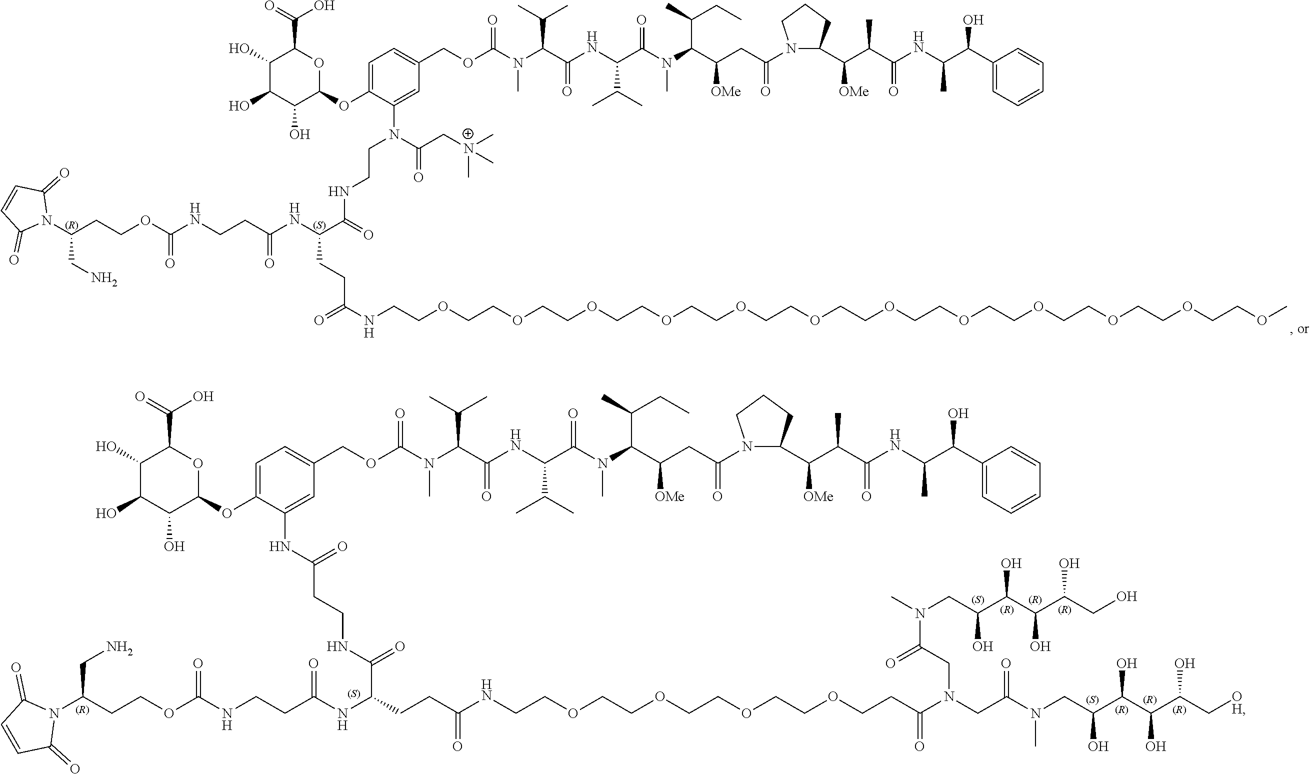
or a pharmaceutically acceptable salt, tautomer, solvate, or stereoisomer thereof.
